(19) United States
(12) Patent Application Publication
(10) Pub. No.: US 2002/0132237 A1
(43) Pub. Date: Sep. 19, 2002

(54) Compositions and methods for the therapy and diagnosis of ovarian cancer

(75) Inventors: Paul A. Algate, Issaquah, WA (US); Robert Jones, Seattle, WA (US); Susan L. Harlocker, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 09/867,701

(22) Filed: May 29, 2001

Related U.S. Application Data
(60) Provisional application No. 60/207,484, filed on May 26, 2000.

(51) Int. Cl. C12Q 1/68; C12P 19/34

(52) U.S. Cl. 435/6; 435/91.2

(57) ABSTRACT

Compositions and methods for the therapy and diagnosis of cancer, particularly ovarian cancer, are disclosed. Illustrative compositions comprise one or more ovarian tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly ovarian cancer.

### CROSS REFERENCE TO RELATED APPLICATIONS

This application is related to U.S. Provisional Application No. 60/207,484 filed on May 26, 2000, which is incorporated in its entirety herein by reference.

### STATEMENT REGARDING SEQUENCE LISTING

The Sequence Listing associated with this application is provided on CD-ROM in lieu of a paper copy, and is hereby incorporated by reference into the specification. Three CD-ROMs are provided containing identical copies of the sequence listing: CD-ROM No. 1 is labeled Copy No. 1, contains the file 597.app.txt which is 6.0 MB and created on May 29, 2001; CD-ROM No. 2 is labeled Copy No. 2, contains the file 497.app.txt which is 6.0 MB and created on May 29, 2001; CD-ROM No. 3 is labeled Computer Readable Form, contains the file 497.app.txt which is 6.0 MD and created on May 29, 2001.

### TECHNICAL FIELD OF THE INVENTION

The present invention relates generally to therapy and diagnosis of cancer, such as ovarian cancer. The invention is more specifically related to polypeptides, comprising at least a portion of an ovarian tumor protein, and to polynucleotides encoding such polypeptides. Such polypeptides and polynucleotides are useful in pharmaceutical compositions, e.g., vaccines, and other compositions for the diagnosis and treatment of ovarian cancer.

### BACKGROUND OF THE INVENTION

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Ovarian cancer is a significant health problem for women in the United States and throughout the world. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Immunotherapies have the potential to substantially improve cancer treatment and survival. Such therapies may involve the generation or enhancement of an immune response to an ovarian carcinoma antigen. However, to date, relatively few ovarian carcinoma antigens are known and the generation of an immune response against such antigens has not been shown to be therapeutically beneficial.

Accordingly, there is a need in the art for improved methods for identifying ovarian tumor antigens and for using such antigens in the therapy of ovarian cancer. The present invention fulfills these needs and further provides other related advantages.

In spite of considerable research into therapies for these and other cancers, ovarian cancer remains difficult to diagnose and treat effectively. Accordingly, there is a need in the art for improved methods for detecting and treating such cancers. The present invention fulfills these needs and further provides other related advantages.

### SUMMARY OF THE INVENTION

In one aspect, the present invention provides polynucleotide compositions comprising a sequence selected from the group consisting of:

(a) sequences provided in SEQ ID NO: 1-10,912;

(b) complements of the sequences provided in SEQ ID NO: 1-10,912;

(c) sequences consisting of at least 20, 25, 30, 35, 40, 45, 50, 75 and 100 contiguous residues of a sequence provided in SEQ ID NO: 1-10,912;

(d) sequences that hybridize to a sequence provided in SEQ ID NO: 1-10,912, under moderate or highly stringent conditions;

(e) sequences having at least 75%, 80%, 85%, 90%, 95%, 96%, 97%, 98% or 99% identity to a sequence of SEQ ID NO: 1-10,912;

(f) degenerate variants of a sequence provided in SEQ ID NO: 1-10,912.

In one preferred embodiment, the polynucleotide compositions of the invention are expressed in at least about 20%, more preferably in at least about 30%, and most preferably in at least about 50% of ovarian tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence encoded by a polynucleotide sequence set forth in SEQ ID NO: 1-10,912.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with ovarian cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with ovarian cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably an ovarian cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NO: 1 represents the cDNA sequence for clone T29202.

SEQ ID NO: 2 represents the cDNA sequence for clone T29204.

SEQ ID NO: 3 represents the cDNA sequence for clone T29205.

SEQ ID NO: 4 represents the cDNA sequence for clone T29208.

SEQ ID NO: 5 represents the cDNA sequence for clone T29210.

SEQ ID NO: 6 represents the cDNA sequence for clone T29221.

SEQ ID NO: 7 represents the cDNA sequence for clone T29223.

SEQ ID NO: 8 represents the cDNA sequence for clone T34666.

SEQ ID NO: 9 represents the cDNA sequence for clone T34668.

SEQ ID NO: 10 represents the cDNA sequence for clone T34674.

SEQ ID NO: 11 represents the cDNA sequence for clone T34677.

SEQ ID NO: 12 represents the cDNA sequence for clone T34681.

SEQ ID NO: 13 represents the cDNA sequence for clone T34684.

SEQ ID NO: 14 represents the cDNA sequence for clone T34698.

SEQ ID NO: 15 represents the cDNA sequence for clone T34699.

SEQ ID NO: 16 represents the cDNA sequence for clone T34703.

SEQ ID NO: 17 represents the cDNA sequence for clone T40200.

SEQ ID NO: 18 represents the cDNA sequence for clone T40207.

SEQ ID NO: 19 represents the cDNA sequence for clone T40208.

SEQ ID NO: 20 represents the cDNA sequence for clone T40210.

SEQ ID NO: 21 represents the cDNA sequence for clone T40217.

SEQ ID NO: 22 represents the cDNA sequence for clone T40224.

SEQ ID NO: 23 represents the cDNA sequence for clone T40226.

SEQ ID NO: 24 represents the cDNA sequence for clone T40227.

SEQ ID NO: 25 represents the cDNA sequence for clone T40228.

SEQ ID NO: 26 represents the cDNA sequence for clone T40231.

SEQ ID NO: 27 represents the cDNA sequence for clone T40232.

SEQ ID NO: 28 represents the cDNA sequence for clone T40237.

SEQ ID NO: 29 represents the cDNA sequence for clone T40241.

SEQ ID NO: 30 represents the cDNA sequence for clone T40243.

SEQ ID NO: 31 represents the cDNA sequence for clone T40245.

SEQ ID NO: 32 represents the cDNA sequence for clone T40246.

SEQ ID NO: 33 represents the cDNA sequence for clone T40247.

SEQ ID NO: 34 represents the cDNA sequence for clone T40251.

SEQ ID NO: 35 represents the cDNA sequence for clone T40258.

SEQ ID NO: 36 represents the cDNA sequence for clone T40259.

SEQ ID NO: 37 represents the cDNA sequence for clone T40260.

SEQ ID NO: 38 represents the cDNA sequence for clone T40264.

SEQ ID NO: 39 represents the cDNA sequence for clone T40265.

SEQ ID NO: 40 represents the cDNA sequence for clone T40273.

SEQ ID NO: 41 represents the cDNA sequence for clone T40274.

SEQ ID NO: 42 represents the cDNA sequence for clone T40283.

SEQ ID NO: 43 represents the cDNA sequence for clone T40284.

SEQ ID NO: 44 represents the cDNA sequence for clone T40287.

SEQ ID NO: 45 represents the cDNA sequence for clone T40288.

SEQ ID NO: 46 represents the cDNA sequence for clone T40289.

SEQ ID NO: 47 represents the cDNA sequence for clone T40291.

SEQ ID NO: 48 represents the cDNA sequence for clone T40292.

SEQ ID NO: 49 represents the cDNA sequence for clone T40293.

SEQ ID NO: 50 represents the cDNA sequence for clone T40295.

SEQ ID NO: 51 represents the cDNA sequence for clone T40296.

SEQ ID NO: 52 represents the cDNA sequence for clone T40299.

SEQ ID NO: 53 represents the cDNA sequence for clone T40300.

SEQ ID NO: 54 represents the cDNA sequence for clone T40305.

SEQ ID NO: 55 represents the cDNA sequence for clone T40306.

SEQ ID NO: 56 represents the cDNA sequence for clone T40311.

SEQ ID NO: 57 represents the cDNA sequence for clone T40319.

SEQ ID NO: 58 represents the cDNA sequence for clone T40320.

SEQ ID NO: 59 represents the cDNA sequence for clone T40321.

SEQ ID NO: 60 represents the cDNA sequence for clone T40326.

SEQ ID NO: 61 represents the cDNA sequence for clone T40329.

SEQ ID NO: 62 represents the cDNA sequence for clone T40342.

SEQ ID NO: 63 represents the cDNA sequence for clone T40344.

SEQ ID NO: 64 represents the cDNA sequence for clone T40348.

SEQ ID NO: 65 represents the cDNA sequence for clone T40354.

SEQ ID NO: 66 represents the cDNA sequence for clone T40359.

SEQ ID NO: 67 represents the cDNA sequence for clone T40361.

SEQ ID NO: 68 represents the cDNA sequence for clone T40366.

SEQ ID NO: 69 represents the cDNA sequence for clone T40368.

SEQ ID NO: 70 represents the cDNA sequence for clone T40369.

SEQ ID NO: 71 represents the cDNA sequence for clone T40374.

SEQ ID NO: 72 represents the cDNA sequence for clone T40381.

SEQ ID NO: 73 represents the cDNA sequence for clone T40383.

SEQ ID NO: 74 represents the cDNA sequence for clone T40386.

SEQ ID NO: 75 represents the cDNA sequence for clone T40389.

SEQ ID NO: 76 represents the cDNA sequence for clone T40390.

SEQ ID NO: 77 represents the cDNA sequence for clone T40397.

SEQ ID NO: 78 represents the cDNA sequence for clone T40403.

SEQ ID NO: 79 represents the cDNA sequence for clone T40411.

SEQ ID NO: 80 represents the cDNA sequence for clone T40412.

SEQ ID NO: 81 represents the cDNA sequence for clone T40413.

SEQ ID NO: 82 represents the cDNA sequence for clone T40414.

SEQ ID NO: 83 represents the cDNA sequence for clone T40417.

SEQ ID NO: 84 represents the cDNA sequence for clone T40420.

SEQ ID NO: 85 represents the cDNA sequence for clone T40424.

SEQ ID NO: 86 represents the cDNA sequence for clone T41068.

SEQ ID NO: 87 represents the cDNA sequence for clone T41069.

SEQ ID NO: 88 represents the cDNA sequence for clone T41074.

SEQ ID NO: 89 represents the cDNA sequence for clone T41075.

SEQ ID NO: 90 represents the cDNA sequence for clone T41080.

SEQ ID NO: 91 represents the cDNA sequence for clone T41090.

SEQ ID NO: 92 represents the cDNA sequence for clone T41092.

SEQ ID NO: 93 represents the cDNA sequence for clone T41093.

SEQ ID NO: 94 represents the cDNA sequence for clone T41094.

SEQ ID NO: 95 represents the cDNA sequence for clone T41097.

SEQ ID NO: 96 represents the cDNA sequence for clone T41106.

SEQ ID NO: 97 represents the cDNA sequence for clone T41108.

SEQ ID NO: 98 represents the cDNA sequence for clone T41112.

SEQ ID NO: 99 represents the cDNA sequence for clone T41116.

SEQ ID NO: 100 represents the cDNA sequence for clone T41117.

SEQ ID NO: 101 represents the cDNA sequence for clone T41120.

SEQ ID NO: 102 represents the cDNA sequence for clone T41121.

SEQ ID NO: 103 represents the cDNA sequence for clone T41122.

SEQ ID NO: 104 represents the cDNA sequence for clone T41123.

SEQ ID NO: 105 represents the cDNA sequence for clone T41124.

SEQ ID NO: 106 represents the cDNA sequence for clone T41126.

SEQ ID NO: 107 represents the cDNA sequence for clone T41127.

SEQ ID NO: 108 represents the cDNA sequence for clone T41128.

SEQ ID NO: 109 represents the cDNA sequence for clone T41129.

SEQ ID NO: 110 represents the cDNA sequence for clone T41132.

SEQ ID NO: 111 represents the cDNA sequence for clone T41134.

SEQ ID NO: 112 represents the cDNA sequence for clone T41139.

SEQ ID NO: 113 represents the cDNA sequence for clone T41141.

SEQ ID NO: 114 represents the cDNA sequence for clone T41145.

SEQ ID NO: 115 represents the cDNA sequence for clone T41148.

SEQ ID NO: 116 represents the cDNA sequence for clone T41149.

SEQ ID NO: 117 represents the cDNA sequence for clone T41151.

SEQ ID NO: 118 represents the cDNA sequence for clone T41152.

SEQ ID NO: 119 represents the cDNA sequence for clone T41153.

SEQ ID NO: 120 represents the cDNA sequence for clone T41154.

SEQ ID NO: 121 represents the cDNA sequence for clone T41155.

SEQ ID NO: 122 represents the cDNA sequence for clone T41157.

SEQ ID NO: 123 represents the cDNA sequence for clone T41159.

SEQ ID NO: 124 represents the cDNA sequence for clone T41160.

SEQ ID NO: 125 represents the cDNA sequence for clone T41161.

SEQ ID NO: 126 represents the cDNA sequence for clone T41164.

SEQ ID NO: 127 represents the cDNA sequence for clone T41169.

SEQ ID NO: 128 represents the cDNA sequence for clone T41174.

SEQ ID NO: 129 represents the cDNA sequence for clone T41177.

SEQ ID NO: 130 represents the cDNA sequence for clone T41185.

SEQ ID NO: 131 represents the cDNA sequence for clone T41189.

SEQ ID NO: 132 represents the cDNA sequence for clone T41190.

SEQ ID NO: 133 represents the cDNA sequence for clone T41191.

SEQ ID NO: 134 represents the cDNA sequence for clone T41201.

SEQ ID NO: 135 represents the cDNA sequence for clone T41203.

SEQ ID NO: 136 represents the cDNA sequence for clone T41207.

SEQ ID NO: 137 represents the cDNA sequence for clone T41216.

SEQ ID NO: 138 represents the cDNA sequence for clone T41220.

SEQ ID NO: 139 represents the cDNA sequence for clone T41223.

SEQ ID NO: 140 represents the cDNA sequence for clone T41226.

SEQ ID NO: 141 represents the cDNA sequence for clone T41232.

SEQ ID NO: 142 represents the cDNA sequence for clone T41234.

SEQ ID NO: 143 represents the cDNA sequence for clone T41236.

SEQ ID NO: 144 represents the cDNA sequence for clone T41238.

SEQ ID NO: 145 represents the cDNA sequence for clone T41244.

SEQ ID NO: 146 represents the cDNA sequence for clone T41250.

SEQ ID NO: 147 represents the cDNA sequence for clone T41257.

SEQ ID NO: 148 represents the cDNA sequence for clone T41258.

SEQ ID NO: 149 represents the cDNA sequence for clone T41260.

SEQ ID NO: 150 represents the cDNA sequence for clone T41262.

SEQ ID NO: 151 represents the cDNA sequence for clone T41264.

SEQ ID NO: 152 represents the cDNA sequence for clone T41267.

SEQ ID NO: 153 represents the cDNA sequence for clone T47075.

SEQ ID NO: 154 represents the cDNA sequence for clone T47077.

SEQ ID NO: 155 represents the cDNA sequence for clone T47079.

SEQ ID NO: 156 represents the cDNA sequence for clone T47083.

SEQ ID NO: 157 represents the cDNA sequence for clone T47092.

SEQ ID NO: 158 represents the cDNA sequence for clone T47093.

SEQ ID NO: 159 represents the cDNA sequence for clone T47096.

SEQ ID NO: 160 represents the cDNA sequence for clone T47097.

SEQ ID NO: 161 represents the cDNA sequence for clone T47098.

SEQ ID NO: 162 represents the cDNA sequence for clone T47099.

SEQ ID NO: 163 represents the cDNA sequence for clone T47100.

SEQ ID NO: 164 represents the cDNA sequence for clone T47101.

SEQ ID NO: 165 represents the cDNA sequence for clone T47102.

SEQ ID NO: 166 represents the cDNA sequence for clone T47103.

SEQ ID NO: 167 represents the cDNA sequence for clone T47106.

SEQ ID NO: 168 represents the cDNA sequence for clone T47112.

SEQ ID NO: 169 represents the cDNA sequence for clone T47113.

SEQ ID NO: 170 represents the cDNA sequence for clone T47114.

SEQ ID NO: 171 represents the cDNA sequence for clone T47124.

SEQ ID NO: 172 represents the cDNA sequence for clone T47125.

SEQ ID NO: 173 represents the cDNA sequence for clone T47128.

SEQ ID NO: 174 represents the cDNA sequence for clone T47129.

SEQ ID NO: 175 represents the cDNA sequence for clone T47130.

SEQ ID NO: 176 represents the cDNA sequence for clone T47139.

SEQ ID NO: 177 represents the cDNA sequence for clone T47140.

SEQ ID NO: 178 represents the cDNA sequence for clone T47141.

SEQ ID NO: 179 represents the cDNA sequence for clone T47142.

SEQ ID NO: 180 represents the cDNA sequence for clone T47148.

SEQ ID NO: 181 represents the cDNA sequence for clone T47149.

SEQ ID NO: 182 represents the cDNA sequence for clone T47150.

SEQ ID NO: 183 represents the cDNA sequence for clone T47151.

SEQ ID NO: 184 represents the cDNA sequence for clone T47156.

SEQ ID NO: 185 represents the cDNA sequence for clone T47157.

SEQ ID NO: 186 represents the cDNA sequence for clone T47160.

SEQ ID NO: 187 represents the cDNA sequence for clone T47162.

SEQ ID NO: 188 represents the cDNA sequence for clone T47163.

SEQ ID NO: 189 represents the cDNA sequence for clone T47164.

SEQ ID NO: 190 represents the cDNA sequence for clone T47168.

SEQ ID NO: 191 represents the cDNA sequence for clone T47171.

SEQ ID NO: 192 represents the cDNA sequence for clone T47172.

SEQ ID NO: 193 represents the cDNA sequence for clone T47176.

SEQ ID NO: 194 represents the cDNA sequence for clone T47177.

SEQ ID NO: 195 represents the cDNA sequence for clone T47180.

SEQ ID NO: 196 represents the cDNA sequence for clone T47181.

SEQ ID NO: 197 represents the cDNA sequence for clone T47185.

SEQ ID NO: 198 represents the cDNA sequence for clone T47187.

SEQ ID NO: 199 represents the cDNA sequence for clone T47188.

SEQ ID NO: 200 represents the cDNA sequence for clone T47194.

SEQ ID NO: 201 represents the cDNA sequence for clone T47195.

SEQ ID NO: 202 represents the cDNA sequence for clone T47197.

SEQ ID NO: 203 represents the cDNA sequence for clone T47205.

SEQ ID NO: 204 represents the cDNA sequence for clone T47206.

SEQ ID NO: 205 represents the cDNA sequence for clone T47208.

SEQ ID NO: 206 represents the cDNA sequence for clone T47209.

SEQ ID NO: 207 represents the cDNA sequence for clone T47214.

SEQ ID NO: 208 represents the cDNA sequence for clone T47215.

SEQ ID NO: 209 represents the cDNA sequence for clone T47218.

SEQ ID NO: 210 represents the cDNA sequence for clone T47219.

SEQ ID NO: 211 represents the cDNA sequence for clone T47220.

SEQ ID NO: 212 represents the cDNA sequence for clone T47221.

SEQ ID NO: 213 represents the cDNA sequence for clone T47222.

SEQ ID NO: 214 represents the cDNA sequence for clone T47223.

SEQ ID NO: 215 represents the cDNA sequence for clone T47224.

SEQ ID NO: 216 represents the cDNA sequence for clone T47231.

SEQ ID NO: 217 represents the cDNA sequence for clone T47232.

SEQ ID NO: 218 represents the cDNA sequence for clone T47235.

SEQ ID NO: 219 represents the cDNA sequence for clone T47236.

SEQ ID NO: 220 represents the cDNA sequence for clone T47247.

SEQ ID NO: 221 represents the cDNA sequence for clone T47248.

SEQ ID NO: 222 represents the cDNA sequence for clone T47253.

SEQ ID NO: 223 represents the cDNA sequence for clone T47254.

SEQ ID NO: 224 represents the cDNA sequence for clone T47255.

SEQ ID NO: 225 represents the cDNA sequence for clone T47256.

SEQ ID NO: 226 represents the cDNA sequence for clone T47259.

SEQ ID NO: 227 represents the cDNA sequence for clone T47260.

SEQ ID NO: 228 represents the cDNA sequence for clone T47261.

SEQ ID NO: 229 represents the cDNA sequence for clone T47262.

SEQ ID NO: 230 represents the cDNA sequence for clone T47273.

SEQ ID NO: 231 represents the cDNA sequence for clone T47274.

SEQ ID NO: 232 represents the cDNA sequence for clone T47279.

SEQ ID NO: 233 represents the cDNA sequence for clone T47280.

SEQ ID NO: 234 represents the cDNA sequence for clone T47284.

SEQ ID NO: 235 represents the cDNA sequence for clone T47285.

SEQ ID NO: 236 represents the cDNA sequence for clone T47286.

SEQ ID NO: 237 represents the cDNA sequence for clone T47289.

SEQ ID NO: 238 represents the cDNA sequence for clone T47290.

SEQ ID NO: 239 represents the cDNA sequence for clone T47291.

SEQ ID NO: 240 represents the cDNA sequence for clone T49706.

SEQ ID NO: 241 represents the cDNA sequence for clone T49707.

SEQ ID NO: 242 represents the cDNA sequence for clone T49723.

SEQ ID NO: 243 represents the cDNA sequence for clone T49724.

SEQ ID NO: 244 represents the cDNA sequence for clone T49730.

SEQ ID NO: 245 represents the cDNA sequence for clone T49744.

SEQ ID NO: 246 represents the cDNA sequence for clone T49747.

SEQ ID NO: 247 represents the cDNA sequence for clone T49748.

SEQ ID NO: 248 represents the cDNA sequence for clone T49751.

SEQ ID NO: 249 represents the cDNA sequence for clone T49752.

SEQ ID NO: 250 represents the cDNA sequence for clone T49759.

SEQ ID NO: 251 represents the cDNA sequence for clone T49760.

SEQ ID NO: 252 represents the cDNA sequence for clone T49765.

SEQ ID NO: 253 represents the cDNA sequence for clone T49766.

SEQ ID NO: 254 represents the cDNA sequence for clone T49769.

SEQ ID NO: 255 represents the cDNA sequence for clone T49770.

SEQ ID NO: 256 represents the cDNA sequence for clone T49775.

SEQ ID NO: 257 represents the cDNA sequence for clone T49782.

SEQ ID NO: 258 represents the cDNA sequence for clone T49783.

SEQ ID NO: 259 represents the cDNA sequence for clone T49785.

SEQ ID NO: 260 represents the cDNA sequence for clone T49786.

SEQ ID NO: 261 represents the cDNA sequence for clone T49787.

SEQ ID NO: 262 represents the cDNA sequence for clone T49788.

SEQ ID NO: 263 represents the cDNA sequence for clone T49789.

SEQ ID NO: 264 represents the cDNA sequence for clone T50099.

SEQ ID NO: 265 represents the cDNA sequence for clone T50103.

SEQ ID NO: 266 represents the cDNA sequence for clone T50105.

SEQ ID NO: 267 represents the cDNA sequence for clone T50111.

SEQ ID NO: 268 represents the cDNA sequence for clone T50112.

SEQ ID NO: 269 represents the cDNA sequence for clone T50113.

SEQ ID NO: 270 represents the cDNA sequence for clone T50116.

SEQ ID NO: 271 represents the cDNA sequence for clone T50118.

SEQ ID NO: 272 represents the cDNA sequence for clone T50121.

SEQ ID NO: 273 represents the cDNA sequence for clone T50122.

SEQ ID NO: 274 represents the cDNA sequence for clone T50123.

SEQ ID NO: 275 represents the cDNA sequence for clone T50125.

SEQ ID NO: 276 represents the cDNA sequence for clone T50126.

SEQ ID NO: 277 represents the cDNA sequence for clone T50127.

SEQ ID NO: 278 represents the cDNA sequence for clone T50128.

SEQ ID NO: 279 represents the cDNA sequence for clone T50131.

SEQ ID NO: 280 represents the cDNA sequence for clone T50132.

SEQ ID NO: 281 represents the cDNA sequence for clone T50137.

SEQ ID NO: 282 represents the cDNA sequence for clone T50138.

SEQ ID NO: 283 represents the cDNA sequence for clone T50141.

SEQ ID NO: 284 represents the cDNA sequence for clone T50143.

SEQ ID NO: 285 represents the cDNA sequence for clone T50151.

SEQ ID NO: 286 represents the cDNA sequence for clone T50152.

SEQ ID NO: 287 represents the cDNA sequence for clone T50157.

SEQ ID NO: 288 represents the cDNA sequence for clone T50163.

SEQ ID NO: 289 represents the cDNA sequence for clone T50164.

SEQ ID NO: 290 represents the cDNA sequence for clone T50165.

SEQ ID NO: 291 represents the cDNA sequence for clone T50169.

SEQ ID NO: 292 represents the cDNA sequence for clone T50172.

SEQ ID NO: 293 represents the cDNA sequence for clone T50173.

SEQ ID NO: 294 represents the cDNA sequence for clone T50174.

SEQ ID NO: 295 represents the cDNA sequence for clone T50176.

SEQ ID NO: 296 represents the cDNA sequence for clone T50177.

SEQ ID NO: 297 represents the cDNA sequence for clone T50178.

SEQ ID NO: 298 represents the cDNA sequence for clone T50179.

SEQ ID NO: 299 represents the cDNA sequence for clone T50181.

SEQ ID NO: 300 represents the cDNA sequence for clone T50184.

SEQ ID NO: 301 represents the cDNA sequence for clone T50185.

SEQ ID NO: 302 represents the cDNA sequence for clone T50191.

SEQ ID NO: 303 represents the cDNA sequence for clone T50192.

SEQ ID NO: 304 represents the cDNA sequence for clone T50193.

SEQ ID NO: 305 represents the cDNA sequence for clone T50197.

SEQ ID NO: 306 represents the cDNA sequence for clone T50205.

SEQ ID NO: 307 represents the cDNA sequence for clone T50207.

SEQ ID NO: 308 represents the cDNA sequence for clone T50209.

SEQ ID NO: 309 represents the cDNA sequence for clone T50211.

SEQ ID NO: 310 represents the cDNA sequence for clone T50212.

SEQ ID NO: 311 represents the cDNA sequence for clone T50213.

SEQ ID NO: 312 represents the cDNA sequence for clone T50218.

SEQ ID NO: 313 represents the cDNA sequence for clone T50225.

SEQ ID NO: 314 represents the cDNA sequence for clone T50226.

SEQ ID NO: 315 represents the cDNA sequence for clone T50229.

SEQ ID NO: 316 represents the cDNA sequence for clone T50231.

SEQ ID NO: 317 represents the cDNA sequence for clone T50232.

SEQ ID NO: 318 represents the cDNA sequence for clone T50235.

SEQ ID NO: 319 represents the cDNA sequence for clone T50238.

SEQ ID NO: 320 represents the cDNA sequence for clone T50240.

SEQ ID NO: 321 represents the cDNA sequence for clone T50244.

SEQ ID NO: 322 represents the cDNA sequence for clone T50247.

SEQ ID NO: 323 represents the cDNA sequence for clone T50248.

SEQ ID NO: 324 represents the cDNA sequence for clone T50251.

SEQ ID NO: 325 represents the cDNA sequence for clone T50255.

SEQ ID NO: 326 represents the cDNA sequence for clone T50256.

SEQ ID NO: 327 represents the cDNA sequence for clone T50261.

SEQ ID NO: 328 represents the cDNA sequence for clone T50266.

SEQ ID NO: 329 represents the cDNA sequence for clone T50273.

SEQ ID NO: 330 represents the cDNA sequence for clone T50276.

SEQ ID NO: 331 represents the cDNA sequence for clone T50277.

SEQ ID NO: 332 represents the cDNA sequence for clone T50352.

SEQ ID NO: 333 represents the cDNA sequence for clone T50355.

SEQ ID NO: 334 represents the cDNA sequence for clone T50357.

SEQ ID NO: 335 represents the cDNA sequence for clone T50358.

SEQ ID NO: 336 represents the cDNA sequence for clone T50360.

SEQ ID NO: 337 represents the cDNA sequence for clone T50370.

SEQ ID NO: 338 represents the cDNA sequence for clone T50376.

SEQ ID NO: 339 represents the cDNA sequence for clone T50377.

SEQ ID NO: 340 represents the cDNA sequence for clone T50424.

SEQ ID NO: 341 represents the cDNA sequence for clone T50425.

SEQ ID NO: 342 represents the cDNA sequence for clone T50426.

SEQ ID NO: 343 represents the cDNA sequence for clone T50432.

SEQ ID NO: 344 represents the cDNA sequence for clone T50438.

SEQ ID NO: 345 represents the cDNA sequence for clone T50439.

SEQ ID NO: 346 represents the cDNA sequence for clone T50440.

SEQ ID NO: 347 represents the cDNA sequence for clone T50442.

SEQ ID NO: 348 represents the cDNA sequence for clone T50443.

SEQ ID NO: 349 represents the cDNA sequence for clone T50444.

SEQ ID NO: 350 represents the cDNA sequence for clone T50448.

SEQ ID NO: 351 represents the cDNA sequence for clone T50488.

SEQ ID NO: 352 represents the cDNA sequence for clone T50489.

SEQ ID NO: 353 represents the cDNA sequence for clone T50492.

SEQ ID NO: 354 represents the cDNA sequence for clone T50505.

SEQ ID NO: 355 represents the cDNA sequence for clone T50506.

SEQ ID NO: 356 represents the cDNA sequence for clone T50509.

SEQ ID NO: 357 represents the cDNA sequence for clone T50513.

SEQ ID NO: 358 represents the cDNA sequence for clone T50514.

SEQ ID NO: 359 represents the cDNA sequence for clone T50555.

SEQ ID NO: 360 represents the cDNA sequence for clone T50556.

SEQ ID NO: 361 represents the cDNA sequence for clone T50557.

SEQ ID NO: 362 represents the cDNA sequence for clone T50560.

SEQ ID NO: 363 represents the cDNA sequence for clone T50568.

SEQ ID NO: 364 represents the cDNA sequence for clone T50570.

SEQ ID NO: 365 represents the cDNA sequence for clone T50571.

SEQ ID NO: 366 represents the cDNA sequence for clone T50572.

SEQ ID NO: 367 represents the cDNA sequence for clone T50576.

SEQ ID NO: 368 represents the cDNA sequence for clone T50577.

SEQ ID NO: 369 represents the cDNA sequence for clone T50578.

SEQ ID NO: 370 represents the cDNA sequence for clone T50582.

SEQ ID NO: 371 represents the cDNA sequence for clone T50622.

SEQ ID NO: 372 represents the cDNA sequence for clone T50623.

SEQ ID NO: 373 represents the cDNA sequence for clone T50627.

SEQ ID NO: 374 represents the cDNA sequence for clone T50632.

SEQ ID NO: 375 represents the cDNA sequence for clone T50637.

SEQ ID NO: 376 represents the cDNA sequence for clone T50641.

SEQ ID NO: 377 represents the cDNA sequence for clone T50642.

SEQ ID NO: 378 represents the cDNA sequence for clone T50644.

SEQ ID NO: 379 represents the cDNA sequence for clone T50648.

SEQ ID NO: 380 represents the cDNA sequence for clone T50649.

SEQ ID NO: 381 represents the cDNA sequence for clone T50971.

SEQ ID NO: 382 represents the cDNA sequence for clone T50973.

SEQ ID NO: 383 represents the cDNA sequence for clone T50974.

SEQ ID NO: 384 represents the cDNA sequence for clone T50982.

SEQ ID NO: 385 represents the cDNA sequence for clone T50984.

SEQ ID NO: 386 represents the cDNA sequence for clone T50986.

SEQ ID NO: 387 represents the cDNA sequence for clone T50989.

SEQ ID NO: 388 represents the cDNA sequence for clone T50991.

SEQ ID NO: 389 represents the cDNA sequence for clone T50992.

SEQ ID NO: 390 represents the cDNA sequence for clone T50994.

SEQ ID NO: 391 represents the cDNA sequence for clone T50995.

SEQ ID NO: 392 represents the cDNA sequence for clone T50996.

SEQ ID NO: 393 represents the cDNA sequence for clone T50998.

SEQ ID NO: 394 represents the cDNA sequence for clone T51002.

SEQ ID NO: 395 represents the cDNA sequence for clone T51004.

SEQ ID NO: 396 represents the cDNA sequence for clone T51008.

SEQ ID NO: 397 represents the cDNA sequence for clone T51009.

SEQ ID NO: 398 represents the cDNA sequence for clone T51021.

SEQ ID NO: 399 represents the cDNA sequence for clone T51026.

SEQ ID NO: 400 represents the cDNA sequence for clone T51028.

SEQ ID NO: 401 represents the cDNA sequence for clone T51030.

SEQ ID NO: 402 represents the cDNA sequence for clone T51031.

SEQ ID NO: 403 represents the cDNA sequence for clone T51034.

SEQ ID NO: 404 represents the cDNA sequence for clone T51037.

SEQ ID NO: 405 represents the cDNA sequence for clone T51045.

SEQ ID NO: 406 represents the cDNA sequence for clone T51046.

SEQ ID NO: 407 represents the cDNA sequence for clone T51049.

SEQ ID NO: 408 represents the cDNA sequence for clone T51052.

SEQ ID NO: 409 represents the cDNA sequence for clone T51067.

SEQ ID NO: 410 represents the cDNA sequence for clone T51068.

SEQ ID NO: 411 represents the cDNA sequence for clone T51080.

SEQ ID NO: 412 represents the cDNA sequence for clone T51082.

SEQ ID NO: 413 represents the cDNA sequence for clone T51083.

SEQ ID NO: 414 represents the cDNA sequence for clone T51086.

SEQ ID NO: 415 represents the cDNA sequence for clone T51094.

SEQ ID NO: 416 represents the cDNA sequence for clone T51099.

SEQ ID NO: 417 represents the cDNA sequence for clone T51106.

SEQ ID NO: 418 represents the cDNA sequence for clone T51113.

SEQ ID NO: 419 represents the cDNA sequence for clone T51114.

SEQ ID NO: 420 represents the cDNA sequence for clone T51120.

SEQ ID NO: 421 represents the cDNA sequence for clone T51817.

SEQ ID NO: 422 represents the cDNA sequence for clone T51818.

SEQ ID NO: 423 represents the cDNA sequence for clone T51819.

SEQ ID NO: 424 represents the cDNA sequence for clone T51822.

SEQ ID NO: 425 represents the cDNA sequence for clone T51823.

SEQ ID NO: 426 represents the cDNA sequence for clone T51825.

SEQ ID NO: 427 represents the cDNA sequence for clone T51826.

SEQ ID NO: 428 represents the cDNA sequence for clone T51829.

SEQ ID NO: 429 represents the cDNA sequence for clone T51832.

SEQ ID NO: 430 represents the cDNA sequence for clone T51842.

SEQ ID NO: 431 represents the cDNA sequence for clone T51843.

SEQ ID NO: 432 represents the cDNA sequence for clone T51848.

SEQ ID NO: 433 represents the cDNA sequence for clone T51850.

SEQ ID NO: 434 represents the cDNA sequence for clone T51851.

SEQ ID NO: 435 represents the cDNA sequence for clone T51854.

SEQ ID NO: 436 represents the cDNA sequence for clone T51857.

SEQ ID NO: 437 represents the cDNA sequence for clone T51861.

SEQ ID NO: 438 represents the cDNA sequence for clone T51867.

SEQ ID NO: 439 represents the cDNA sequence for clone T51870.

SEQ ID NO: 440 represents the cDNA sequence for clone T51871.

SEQ ID NO: 441 represents the cDNA sequence for clone T51874.

SEQ ID NO: 442 represents the cDNA sequence for clone T51877.

SEQ ID NO: 443 represents the cDNA sequence for clone T51883.

SEQ ID NO: 444 represents the cDNA sequence for clone T51888.

SEQ ID NO: 445 represents the cDNA sequence for clone T51897.

SEQ ID NO: 446 represents the cDNA sequence for clone T51898.

SEQ ID NO: 447 represents the cDNA sequence for clone T51902.

SEQ ID NO: 448 represents the cDNA sequence for clone T51905.

SEQ ID NO: 449 represents the cDNA sequence for clone T52744.

SEQ ID NO: 450 represents the cDNA sequence for clone T52745.

SEQ ID NO: 451 represents the cDNA sequence for clone T52747.

SEQ ID NO: 452 represents the cDNA sequence for clone T52748.

SEQ ID NO: 453 represents the cDNA sequence for clone T52749.

SEQ ID NO: 454 represents the cDNA sequence for clone T52750.

SEQ ID NO: 455 represents the cDNA sequence for clone T52752.

SEQ ID NO: 456 represents the cDNA sequence for clone T52753.

SEQ ID NO: 457 represents the cDNA sequence for clone T52754.

SEQ ID NO: 458 represents the cDNA sequence for clone T52755.

SEQ ID NO: 459 represents the cDNA sequence for clone T52760.

SEQ ID NO: 460 represents the cDNA sequence for clone T52761.

SEQ ID NO: 461 represents the cDNA sequence for clone T52764.

SEQ ID NO: 462 represents the cDNA sequence for clone T52767.

SEQ ID NO: 463 represents the cDNA sequence for clone T52774.

SEQ ID NO: 464 represents the cDNA sequence for clone T52776.

SEQ ID NO: 465 represents the cDNA sequence for clone T52782.

SEQ ID NO: 466 represents the cDNA sequence for clone T52783.

SEQ ID NO: 467 represents the cDNA sequence for clone T52787.

SEQ ID NO: 468 represents the cDNA sequence for clone T52788.

SEQ ID NO: 469 represents the cDNA sequence for clone T52789.

SEQ ID NO: 470 represents the cDNA sequence for clone T52796.

SEQ ID NO: 471 represents the cDNA sequence for clone T52799.

SEQ ID NO: 472 represents the cDNA sequence for clone T52800.

SEQ ID NO: 473 represents the cDNA sequence for clone T52802.

SEQ ID NO: 474 represents the cDNA sequence for clone T52803.

SEQ ID NO: 475 represents the cDNA sequence for clone T52807.

SEQ ID NO: 476 represents the cDNA sequence for clone T52808.

SEQ ID NO: 477 represents the cDNA sequence for clone T52809.

SEQ ID NO: 478 represents the cDNA sequence for clone T52810.

SEQ ID NO: 479 represents the cDNA sequence for clone T52812.

SEQ ID NO: 480 represents the cDNA sequence for clone T52815.

SEQ ID NO: 481 represents the cDNA sequence for clone T52816.

SEQ ID NO: 482 represents the cDNA sequence for clone T52819.

SEQ ID NO: 483 represents the cDNA sequence for clone T52820.

SEQ ID NO: 484 represents the cDNA sequence for clone T52821.

SEQ ID NO: 485 represents the cDNA sequence for clone T52828.

SEQ ID NO: 486 represents the cDNA sequence for clone T52832.

SEQ ID NO: 487 represents the cDNA sequence for clone T52837.

SEQ ID NO: 488 represents the cDNA sequence for clone T52838.

SEQ ID NO: 489 represents the cDNA sequence for clone T52842.

SEQ ID NO: 490 represents the cDNA sequence for clone T52843.

SEQ ID NO: 491 represents the cDNA sequence for clone T52844.

SEQ ID NO: 492 represents the cDNA sequence for clone T52847.

SEQ ID NO: 493 represents the cDNA sequence for clone T52848.

SEQ ID NO: 494 represents the cDNA sequence for clone T52849.

SEQ ID NO: 495 represents the cDNA sequence for clone T52851.

SEQ ID NO: 496 represents the cDNA sequence for clone T52862.

SEQ ID NO: 497 represents the cDNA sequence for clone T52863.

SEQ ID NO: 498 represents the cDNA sequence for clone T52868.

SEQ ID NO: 499 represents the cDNA sequence for clone T52869.

SEQ ID NO: 500 represents the cDNA sequence for clone T52873.

SEQ ID NO: 501 represents the cDNA sequence for clone T52878.

SEQ ID NO: 502 represents the cDNA sequence for clone T52886.

SEQ ID NO: 503 represents the cDNA sequence for clone T52887.

SEQ ID NO: 504 represents the cDNA sequence for clone T52890.

SEQ ID NO: 505 represents the cDNA sequence for clone T52892.

SEQ ID NO: 506 represents the cDNA sequence for clone T52900.

SEQ ID NO: 507 represents the cDNA sequence for clone T52906.

SEQ ID NO: 508 represents the cDNA sequence for clone T52907.

SEQ ID NO: 509 represents the cDNA sequence for clone T52916.

SEQ ID NO: 510 represents the cDNA sequence for clone T52917.

SEQ ID NO: 511 represents the cDNA sequence for clone T52920.

SEQ ID NO: 512 represents the cDNA sequence for clone T52922.

SEQ ID NO: 513 represents the cDNA sequence for clone T52923.

SEQ ID NO: 514 represents the cDNA sequence for clone T52924.

SEQ ID NO: 515 represents the cDNA sequence for clone T52925.

SEQ ID NO: 516 represents the cDNA sequence for clone T52926.

SEQ ID NO: 517 represents the cDNA sequence for clone T52932.

SEQ ID NO: 518 represents the cDNA sequence for clone T52933.

SEQ ID NO: 519 represents the cDNA sequence for clone T52934.

SEQ ID NO: 520 represents the cDNA sequence for clone T52935.

SEQ ID NO: 521 represents the cDNA sequence for clone T52944.

SEQ ID NO: 522 represents the cDNA sequence for clone T52945.

SEQ ID NO: 523 represents the cDNA sequence for clone T52954.

SEQ ID NO: 524 represents the cDNA sequence for clone T52955.

SEQ ID NO: 525 represents the cDNA sequence for clone T52957.

SEQ ID NO: 526 represents the cDNA sequence for clone T52958.

SEQ ID NO: 527 represents the cDNA sequence for clone T52959.

SEQ ID NO: 528 represents the cDNA sequence for clone T52960.

SEQ ID NO: 529 represents the cDNA sequence for clone T52961.

SEQ ID NO: 530 represents the cDNA sequence for clone T52966.

SEQ ID NO: 531 represents the cDNA sequence for clone T52967.

SEQ ID NO: 532 represents the cDNA sequence for clone T52968.

SEQ ID NO: 533 represents the cDNA sequence for clone T52970.

SEQ ID NO: 534 represents the cDNA sequence for clone T52971.

SEQ ID NO: 535 represents the cDNA sequence for clone T52978.

SEQ ID NO: 536 represents the cDNA sequence for clone T52982.

SEQ ID NO: 537 represents the cDNA sequence for clone T52983.

SEQ ID NO: 538 represents the cDNA sequence for clone T52984.

SEQ ID NO: 539 represents the cDNA sequence for clone T52985.

SEQ ID NO: 540 represents the cDNA sequence for clone T52992.

SEQ ID NO: 541 represents the cDNA sequence for clone T52993.

SEQ ID NO: 542 represents the cDNA sequence for clone T52996.

SEQ ID NO: 543 represents the cDNA sequence for clone T52997.

SEQ ID NO: 544 represents the cDNA sequence for clone T52999.

SEQ ID NO: 545 represents the cDNA sequence for clone T53000.

SEQ ID NO: 546 represents the cDNA sequence for clone T53001.

SEQ ID NO: 547 represents the cDNA sequence for clone T53002.

SEQ ID NO: 548 represents the cDNA sequence for clone T53003.

SEQ ID NO: 549 represents the cDNA sequence for clone T53006.

SEQ ID NO: 550 represents the cDNA sequence for clone T53007.

SEQ ID NO: 551 represents the cDNA sequence for clone T53015.

SEQ ID NO: 552 represents the cDNA sequence for clone T53016.

SEQ ID NO: 553 represents the cDNA sequence for clone T53018.

SEQ ID NO: 554 represents the cDNA sequence for clone T53021.

SEQ ID NO: 555 represents the cDNA sequence for clone T53022.

SEQ ID NO: 556 represents the cDNA sequence for clone T53023.

SEQ ID NO: 557 represents the cDNA sequence for clone T53024.

SEQ ID NO: 558 represents the cDNA sequence for clone T53027.

SEQ ID NO: 559 represents the cDNA sequence for clone T47159.

SEQ ID NO: 560 represents the cDNA sequence for clone T55330.

SEQ ID NO: 561 represents the cDNA sequence for clone T55335.

SEQ ID NO: 562 represents the cDNA sequence for clone T55340.

SEQ ID NO: 563 represents the cDNA sequence for clone T55342.

SEQ ID NO: 564 represents the cDNA sequence for clone T55343.

SEQ ID NO: 565 represents the cDNA sequence for clone T55349.

SEQ ID NO: 566 represents the cDNA sequence for clone T55351.

SEQ ID NO: 567 represents the cDNA sequence for clone T55353.

SEQ ID NO: 568 represents the cDNA sequence for clone T55355.

SEQ ID NO: 569 represents the cDNA sequence for clone T56172.

SEQ ID NO: 570 represents the cDNA sequence for clone T56175.

SEQ ID NO: 571 represents the cDNA sequence for clone T56179.

SEQ ID NO: 572 represents the cDNA sequence for clone T56182.

SEQ ID NO: 573 represents the cDNA sequence for clone T56183.

SEQ ID NO: 574 represents the cDNA sequence for clone T56189.

SEQ ID NO: 575 represents the cDNA sequence for clone T56193.

SEQ ID NO: 576 represents the cDNA sequence for clone T57460.

SEQ ID NO: 577 represents the cDNA sequence for clone T57465.

SEQ ID NO: 578 represents the cDNA sequence for clone T57466.

SEQ ID NO: 579 represents the cDNA sequence for clone T57469.

SEQ ID NO: 580 represents the cDNA sequence for clone T57470.

SEQ ID NO: 581 represents the cDNA sequence for clone T57472.

SEQ ID NO: 582 represents the cDNA sequence for clone T57477.

SEQ ID NO: 583 represents the cDNA sequence for clone T57481.

SEQ ID NO: 584 represents the cDNA sequence for clone T57484.

SEQ ID NO: 585 represents the cDNA sequence for clone T57490.

SEQ ID NO: 586 represents the cDNA sequence for clone T57493.

SEQ ID NO: 587 represents the cDNA sequence for clone T57495.

SEQ ID NO: 588 represents the cDNA sequence for clone T57497.

SEQ ID NO: 589 represents the cDNA sequence for clone T57498.

SEQ ID NO: 590 represents the cDNA sequence for clone T57500.

SEQ ID NO: 591 represents the cDNA sequence for clone T57509.

SEQ ID NO: 592 represents the cDNA sequence for clone T57510.

SEQ ID NO: 593 represents the cDNA sequence for clone T57511.

SEQ ID NO: 594 represents the cDNA sequence for clone T57513.

SEQ ID NO: 595 represents the cDNA sequence for clone T57514.

SEQ ID NO: 596 represents the cDNA sequence for clone T57518.

SEQ ID NO: 597 represents the cDNA sequence for clone T57520.

SEQ ID NO: 598 represents the cDNA sequence for clone T57525.

SEQ ID NO: 599 represents the cDNA sequence for clone T57527.

SEQ ID NO: 600 represents the cDNA sequence for clone T57532.

SEQ ID NO: 601 represents the cDNA sequence for clone T57540.

SEQ ID NO: 602 represents the cDNA sequence for clone T57545.

SEQ ID NO: 603 represents the cDNA sequence for clone T57551.

SEQ ID NO: 604 represents the cDNA sequence for clone T57557.

SEQ ID NO: 605 represents the cDNA sequence for clone T57558.

SEQ ID NO: 606 represents the cDNA sequence for clone T57559.

SEQ ID NO: 607 represents the cDNA sequence for clone T57560.

SEQ ID NO: 608 represents the cDNA sequence for clone T57564.

SEQ ID NO: 609 represents the cDNA sequence for clone T57566.

SEQ ID NO: 610 represents the cDNA sequence for clone T57571.

SEQ ID NO: 611 represents the cDNA sequence for clone T57572.

SEQ ID NO: 612 represents the cDNA sequence for clone T57574.

SEQ ID NO: 613 represents the cDNA sequence for clone T57575.

SEQ ID NO: 614 represents the cDNA sequence for clone T57581.

SEQ ID NO: 615 represents the cDNA sequence for clone T57585.

SEQ ID NO: 616 represents the cDNA sequence for clone T57586.

SEQ ID NO: 617 represents the cDNA sequence for clone T57589.

SEQ ID NO: 618 represents the cDNA sequence for clone T57595.

SEQ ID NO: 619 represents the cDNA sequence for clone T57597.

SEQ ID NO: 620 represents the cDNA sequence for clone T57605.

SEQ ID NO: 621 represents the cDNA sequence for clone T57608.

SEQ ID NO: 622 represents the cDNA sequence for clone T57611.

SEQ ID NO: 623 represents the cDNA sequence for clone T57612.

SEQ ID NO: 624 represents the cDNA sequence for clone T57613.

SEQ ID NO: 625 represents the cDNA sequence for clone T57616.

SEQ ID NO: 626 represents the cDNA sequence for clone T57623.

SEQ ID NO: 627 represents the cDNA sequence for clone T57631.

SEQ ID NO: 628 represents the cDNA sequence for clone T57635.

SEQ ID NO: 629 represents the cDNA sequence for clone T57636.

SEQ ID NO: 630 represents the cDNA sequence for clone T57640.

SEQ ID NO: 631 represents the cDNA sequence for clone T57641.

SEQ ID NO: 632 represents the cDNA sequence for clone T57642.

SEQ ID NO: 633 represents the cDNA sequence for clone T57650.

SEQ ID NO: 634 represents the cDNA sequence for clone T57655.

SEQ ID NO: 635 represents the cDNA sequence for clone T57656.

SEQ ID NO: 636 represents the cDNA sequence for clone T57659.

SEQ ID NO: 637 represents the cDNA sequence for clone T58334.

SEQ ID NO: 638 represents the cDNA sequence for clone T58335.

SEQ ID NO: 639 represents the cDNA sequence for clone T58337.

SEQ ID NO: 640 represents the cDNA sequence for clone T58344.

SEQ ID NO: 641 represents the cDNA sequence for clone T58347.

SEQ ID NO: 642 represents the cDNA sequence for clone T58348.

SEQ ID NO: 643 represents the cDNA sequence for clone T58351.

SEQ ID NO: 644 represents the cDNA sequence for clone T58353.

SEQ ID NO: 645 represents the cDNA sequence for clone T58354.

SEQ ID NO: 646 represents the cDNA sequence for clone T58357.

SEQ ID NO: 647 represents the cDNA sequence for clone T58358.

SEQ ID NO: 648 represents the cDNA sequence for clone T58362.

SEQ ID NO: 649 represents the cDNA sequence for clone T58366.

SEQ ID NO: 650 represents the cDNA sequence for clone T58367.

SEQ ID NO: 651 represents the cDNA sequence for clone T58368.

SEQ ID NO: 652 represents the cDNA sequence for clone T58371.

SEQ ID NO: 653 represents the cDNA sequence for clone T58375.

SEQ ID NO: 654 represents the cDNA sequence for clone T58378.

SEQ ID NO: 655 represents the cDNA sequence for clone T58379.

SEQ ID NO: 656 represents the cDNA sequence for clone T58385.

SEQ ID NO: 657 represents the cDNA sequence for clone T58386.

SEQ ID NO: 658 represents the cDNA sequence for clone T58388.

SEQ ID NO: 659 represents the cDNA sequence for clone T58391.

SEQ ID NO: 660 represents the cDNA sequence for clone T58392.

SEQ ID NO: 661 represents the cDNA sequence for clone T58396.

SEQ ID NO: 662 represents the cDNA sequence for clone T58399.

SEQ ID NO: 663 represents the cDNA sequence for clone T58402.

SEQ ID NO: 664 represents the cDNA sequence for clone T58403.

SEQ ID NO: 665 represents the cDNA sequence for clone T58407.

SEQ ID NO: 666 represents the cDNA sequence for clone T58408.

SEQ ID NO: 667 represents the cDNA sequence for clone T58415.

SEQ ID NO: 668 represents the cDNA sequence for clone T58416.

SEQ ID NO: 669 represents the cDNA sequence for clone T58417.

SEQ ID NO: 670 represents the cDNA sequence for clone T58421.

SEQ ID NO: 671 represents the cDNA sequence for clone T58424.

SEQ ID NO: 672 represents the cDNA sequence for clone T58425.

SEQ ID NO: 673 represents the cDNA sequence for clone T58428.

SEQ ID NO: 674 represents the cDNA sequence for clone T58429.

SEQ ID NO: 675 represents the cDNA sequence for clone T58434.

SEQ ID NO: 676 represents the cDNA sequence for clone T58436.

SEQ ID NO: 677 represents the cDNA sequence for clone T58438.

SEQ ID NO: 678 represents the cDNA sequence for clone T58440.

SEQ ID NO: 679 represents the cDNA sequence for clone T58444.

SEQ ID NO: 680 represents the cDNA sequence for clone T58449.

SEQ ID NO: 681 represents the cDNA sequence for clone T58450.

SEQ ID NO: 682 represents the cDNA sequence for clone T58452.

SEQ ID NO: 683 represents the cDNA sequence for clone T58456.

SEQ ID NO: 684 represents the cDNA sequence for clone T58457.

SEQ ID NO: 685 represents the cDNA sequence for clone T58459.

SEQ ID NO: 686 represents the cDNA sequence for clone T58460.

SEQ ID NO: 687 represents the cDNA sequence for clone T58461.

SEQ ID NO: 688 represents the cDNA sequence for clone T58466.

SEQ ID NO: 689 represents the cDNA sequence for clone T58468.

SEQ ID NO: 690 represents the cDNA sequence for clone T58472.

SEQ ID NO: 691 represents the cDNA sequence for clone T58474.

SEQ ID NO: 692 represents the cDNA sequence for clone T58480.

SEQ ID NO: 693 represents the cDNA sequence for clone T58484.

SEQ ID NO: 694 represents the cDNA sequence for clone T58486.

SEQ ID NO: 695 represents the cDNA sequence for clone T58490.

SEQ ID NO: 696 represents the cDNA sequence for clone T58491.

SEQ ID NO: 697 represents the cDNA sequence for clone T58492.

SEQ ID NO: 698 represents the cDNA sequence for clone T58494.

SEQ ID NO: 699 represents the cDNA sequence for clone T58495.

SEQ ID NO: 700 represents the cDNA sequence for clone T58500.

SEQ ID NO: 701 represents the cDNA sequence for clone T58501.

SEQ ID NO: 702 represents the cDNA sequence for clone T58505.

SEQ ID NO: 703 represents the cDNA sequence for clone T58506.

SEQ ID NO: 704 represents the cDNA sequence for clone T58507.

SEQ ID NO: 705 represents the cDNA sequence for clone T58510.

SEQ ID NO: 706 represents the cDNA sequence for clone T58513.

SEQ ID NO: 707 represents the cDNA sequence for clone T58514.

SEQ ID NO: 708 represents the cDNA sequence for clone T58521.

SEQ ID NO: 709 represents the cDNA sequence for clone T58522.

SEQ ID NO: 710 represents the cDNA sequence for clone T58524.

SEQ ID NO: 711 represents the cDNA sequence for clone T58526.

SEQ ID NO: 712 represents the cDNA sequence for clone T58527.

SEQ ID NO: 713 represents the cDNA sequence for clone T58528.

SEQ ID NO: 714 represents the cDNA sequence for clone T58531.

SEQ ID NO: 715 represents the cDNA sequence for clone T58533.

SEQ ID NO: 716 represents the cDNA sequence for clone T58534.

SEQ ID NO: 717 represents the cDNA sequence for clone T58538.

SEQ ID NO: 718 represents the cDNA sequence for clone T58541.

SEQ ID NO: 719 represents the cDNA sequence for clone T58542.

SEQ ID NO: 720 represents the cDNA sequence for clone T58543.

SEQ ID NO: 721 represents the cDNA sequence for clone T58550.

SEQ ID NO: 722 represents the cDNA sequence for clone T58551.

SEQ ID NO: 723 represents the cDNA sequence for clone T58555.

SEQ ID NO: 724 represents the cDNA sequence for clone T58557.

SEQ ID NO: 725 represents the cDNA sequence for clone T58559.

SEQ ID NO: 726 represents the cDNA sequence for clone T58563.

SEQ ID NO: 727 represents the cDNA sequence for clone T58566.

SEQ ID NO: 728 represents the cDNA sequence for clone T58570.

SEQ ID NO: 729 represents the cDNA sequence for clone T58572.

SEQ ID NO: 730 represents the cDNA sequence for clone T58573.

SEQ ID NO: 731 represents the cDNA sequence for clone T58577.

SEQ ID NO: 732 represents the cDNA sequence for clone T58585.

SEQ ID NO: 733 represents the cDNA sequence for clone T58588.

SEQ ID NO: 734 represents the cDNA sequence for clone T58590.

SEQ ID NO: 735 represents the cDNA sequence for clone T58591.

SEQ ID NO: 736 represents the cDNA sequence for clone T58594.

SEQ ID NO: 737 represents the cDNA sequence for clone T58598.

SEQ ID NO: 738 represents the cDNA sequence for clone T58599.

SEQ ID NO: 739 represents the cDNA sequence for clone T58968.

SEQ ID NO: 740 represents the cDNA sequence for clone T58969.

SEQ ID NO: 741 represents the cDNA sequence for clone T58976.

SEQ ID NO: 742 represents the cDNA sequence for clone T58977.

SEQ ID NO: 743 represents the cDNA sequence for clone T58983.

SEQ ID NO: 744 represents the cDNA sequence for clone T58985.

SEQ ID NO: 745 represents the cDNA sequence for clone T58989.

SEQ ID NO: 746 represents the cDNA sequence for clone T58990.

SEQ ID NO: 747 represents the cDNA sequence for clone T59305.

SEQ ID NO: 748 represents the cDNA sequence for clone T59309.

SEQ ID NO: 749 represents the cDNA sequence for clone T59315.

SEQ ID NO: 750 represents the cDNA sequence for clone T59319.

SEQ ID NO: 751 represents the cDNA sequence for clone T59321.

SEQ ID NO: 752 represents the cDNA sequence for clone T59324.

SEQ ID NO: 753 represents the cDNA sequence for clone T59325.

SEQ ID NO: 754 represents the cDNA sequence for clone T59326.

SEQ ID NO: 755 represents the cDNA sequence for clone T59330.

SEQ ID NO: 756 represents the cDNA sequence for clone T59337.

SEQ ID NO: 757 represents the cDNA sequence for clone T59338.

SEQ ID NO: 758 represents the cDNA sequence for clone T59344.

SEQ ID NO: 759 represents the cDNA sequence for clone T59345.

SEQ ID NO: 760 represents the cDNA sequence for clone T59347.

SEQ ID NO: 761 represents the cDNA sequence for clone T59349.

SEQ ID NO: 762 represents the cDNA sequence for clone T59353.

SEQ ID NO: 763 represents the cDNA sequence for clone T59354.

SEQ ID NO: 764 represents the cDNA sequence for clone T59355.

SEQ ID NO: 765 represents the cDNA sequence for clone T59361.

SEQ ID NO: 766 represents the cDNA sequence for clone T59362.

SEQ ID NO: 767 represents the cDNA sequence for clone T59363.

SEQ ID NO: 768 represents the cDNA sequence for clone T59368.

SEQ ID NO: 769 represents the cDNA sequence for clone T59370.

SEQ ID NO: 770 represents the cDNA sequence for clone T59371.

SEQ ID NO: 771 represents the cDNA sequence for clone T59373.

SEQ ID NO: 772 represents the cDNA sequence for clone T59374.

SEQ ID NO: 773 represents the cDNA sequence for clone T59375.

SEQ ID NO: 774 represents the cDNA sequence for clone T59376.

SEQ ID NO: 775 represents the cDNA sequence for clone T59377.

SEQ ID NO: 776 represents the cDNA sequence for clone T59379.

SEQ ID NO: 777 represents the cDNA sequence for clone T59380.

SEQ ID NO: 778 represents the cDNA sequence for clone T59383.

SEQ ID NO: 779 represents the cDNA sequence for clone T59389.

SEQ ID NO: 780 represents the cDNA sequence for clone T59390.

SEQ ID NO: 781 represents the cDNA sequence for clone T59391.

SEQ ID NO: 782 represents the cDNA sequence for clone T59392.

SEQ ID NO: 783 represents the cDNA sequence for clone T59407.

SEQ ID NO: 784 represents the cDNA sequence for clone T59410.

SEQ ID NO: 785 represents the cDNA sequence for clone T59420.

SEQ ID NO: 786 represents the cDNA sequence for clone T59423.

SEQ ID NO: 787 represents the cDNA sequence for clone T59427.

SEQ ID NO: 788 represents the cDNA sequence for clone T59431.

SEQ ID NO: 789 represents the cDNA sequence for clone T59432.

SEQ ID NO: 790 represents the cDNA sequence for clone T59433.

SEQ ID NO: 791 represents the cDNA sequence for clone T59436.

SEQ ID NO: 792 represents the cDNA sequence for clone T59439.

SEQ ID NO: 793 represents the cDNA sequence for clone T59442.

SEQ ID NO: 794 represents the cDNA sequence for clone T59443.

SEQ ID NO: 795 represents the cDNA sequence for clone T59444.

SEQ ID NO: 796 represents the cDNA sequence for clone T59451.

SEQ ID NO: 797 represents the cDNA sequence for clone T59455.

SEQ ID NO: 798 represents the cDNA sequence for clone T59459.

SEQ ID NO: 799 represents the cDNA sequence for clone T59460.

SEQ ID NO: 800 represents the cDNA sequence for clone T59461.

SEQ ID NO: 801 represents the cDNA sequence for clone T59464.

SEQ ID NO: 802 represents the cDNA sequence for clone T59470.

SEQ ID NO: 803 represents the cDNA sequence for clone T59471.

SEQ ID NO: 804 represents the cDNA sequence for clone T59472.

SEQ ID NO: 805 represents the cDNA sequence for clone T59475.

SEQ ID NO: 806 represents the cDNA sequence for clone T59476.

SEQ ID NO: 807 represents the cDNA sequence for clone T59479.

SEQ ID NO: 808 represents the cDNA sequence for clone T59481.

SEQ ID NO: 809 represents the cDNA sequence for clone T59483.

SEQ ID NO: 810 represents the cDNA sequence for clone T59486.

SEQ ID NO: 811 represents the cDNA sequence for clone T59487.

SEQ ID NO: 812 represents the cDNA sequence for clone T59488.

SEQ ID NO: 813 represents the cDNA sequence for clone T59491.

SEQ ID NO: 814 represents the cDNA sequence for clone T59493.

SEQ ID NO: 815 represents the cDNA sequence for clone T59494.

SEQ ID NO: 816 represents the cDNA sequence for clone T59498.

SEQ ID NO: 817 represents the cDNA sequence for clone T59499.

SEQ ID NO: 818 represents the cDNA sequence for clone T59505.

SEQ ID NO: 819 represents the cDNA sequence for clone T59509.

SEQ ID NO: 820 represents the cDNA sequence for clone T59513.

SEQ ID NO: 821 represents the cDNA sequence for clone T59517.

SEQ ID NO: 822 represents the cDNA sequence for clone T59525.

SEQ ID NO: 823 represents the cDNA sequence for clone T59528.

SEQ ID NO: 824 represents the cDNA sequence for clone T59529.

SEQ ID NO: 825 represents the cDNA sequence for clone T59531.

SEQ ID NO: 826 represents the cDNA sequence for clone T59534.

SEQ ID NO: 827 represents the cDNA sequence for clone T59536.

SEQ ID NO: 828 represents the cDNA sequence for clone T59537.

SEQ ID NO: 829 represents the cDNA sequence for clone T59538.

SEQ ID NO: 830 represents the cDNA sequence for clone T59540.

SEQ ID NO: 831 represents the cDNA sequence for clone T59541.

SEQ ID NO: 832 represents the cDNA sequence for clone T59542.

SEQ ID NO: 833 represents the cDNA sequence for clone T59543.

SEQ ID NO: 834 represents the cDNA sequence for clone T59546.

SEQ ID NO: 835 represents the cDNA sequence for clone T59550.

SEQ ID NO: 836 represents the cDNA sequence for clone T59551.

SEQ ID NO: 837 represents the cDNA sequence for clone T59555.

SEQ ID NO: 838 represents the cDNA sequence for clone T59557.

SEQ ID NO: 839 represents the cDNA sequence for clone T59559.

SEQ ID NO: 840 represents the cDNA sequence for clone T59561.

SEQ ID NO: 841 represents the cDNA sequence for clone T59563.

SEQ ID NO: 842 represents the cDNA sequence for clone T59566.

SEQ ID NO: 843 represents the cDNA sequence for clone T59567.

SEQ ID NO: 844 represents the cDNA sequence for clone T59570.

SEQ ID NO: 845 represents the cDNA sequence for clone T59572.

SEQ ID NO: 846 represents the cDNA sequence for clone T59575.

SEQ ID NO: 847 represents the cDNA sequence for clone T59578.

SEQ ID NO: 848 represents the cDNA sequence for clone T59581.

SEQ ID NO: 849 represents the cDNA sequence for clone T59584.

SEQ ID NO: 850 represents the cDNA sequence for clone T59585.

SEQ ID NO: 851 represents the cDNA sequence for clone T59588.

SEQ ID NO: 852 represents the cDNA sequence for clone T59589.

SEQ ID NO: 853 represents the cDNA sequence for clone T59590.

SEQ ID NO: 854 represents the cDNA sequence for clone T59593.

SEQ ID NO: 855 represents the cDNA sequence for clone T59595.

SEQ ID NO: 856 represents the cDNA sequence for clone T59596.

SEQ ID NO: 857 represents the cDNA sequence for clone T59597.

SEQ ID NO: 858 represents the cDNA sequence for clone T59598.

SEQ ID NO: 859 represents the cDNA sequence for clone T59600.

SEQ ID NO: 860 represents the cDNA sequence for clone T59602.

SEQ ID NO: 861 represents the cDNA sequence for clone T59604.

SEQ ID NO: 862 represents the cDNA sequence for clone T59605.

SEQ ID NO: 863 represents the cDNA sequence for clone T59607.

SEQ ID NO: 864 represents the cDNA sequence for clone T59608.

SEQ ID NO: 865 represents the cDNA sequence for clone T59610.

SEQ ID NO: 866 represents the cDNA sequence for clone T59621.

SEQ ID NO: 867 represents the cDNA sequence for clone T59650.

SEQ ID NO: 868 represents the cDNA sequence for clone T59651.

SEQ ID NO: 869 represents the cDNA sequence for clone T59652.

SEQ ID NO: 870 represents the cDNA sequence for clone T59654.

SEQ ID NO: 871 represents the cDNA sequence for clone T59655.

SEQ ID NO: 872 represents the cDNA sequence for clone T59660.

SEQ ID NO: 873 represents the cDNA sequence for clone T59662.

SEQ ID NO: 874 represents the cDNA sequence for clone T59663.

SEQ ID NO: 875 represents the cDNA sequence for clone T59666.

SEQ ID NO: 876 represents the cDNA sequence for clone T59669.

SEQ ID NO: 877 represents the cDNA sequence for clone T59670.

SEQ ID NO: 878 represents the cDNA sequence for clone T59672.

SEQ ID NO: 879 represents the cDNA sequence for clone T59714.

SEQ ID NO: 880 represents the cDNA sequence for clone T59717.

SEQ ID NO: 881 represents the cDNA sequence for clone T59718.

SEQ ID NO: 882 represents the cDNA sequence for clone T59730.

SEQ ID NO: 883 represents the cDNA sequence for clone T59782.

SEQ ID NO: 884 represents the cDNA sequence for clone T59783.

SEQ ID NO: 885 represents the cDNA sequence for clone T59784.

SEQ ID NO: 886 represents the cDNA sequence for clone T59786.

SEQ ID NO: 887 represents the cDNA sequence for clone T59787.

SEQ ID NO: 888 represents the cDNA sequence for clone T59788.

SEQ ID NO: 889 represents the cDNA sequence for clone T59790.

SEQ ID NO: 890 represents the cDNA sequence for clone T59791.

SEQ ID NO: 891 represents the cDNA sequence for clone T59792.

SEQ ID NO: 892 represents the cDNA sequence for clone T59793.

SEQ ID NO: 893 represents the cDNA sequence for clone T59795.

SEQ ID NO: 894 represents the cDNA sequence for clone T59796.

SEQ ID NO: 895 represents the cDNA sequence for clone T59799.

SEQ ID NO: 896 represents the cDNA sequence for clone T59803.

SEQ ID NO: 897 represents the cDNA sequence for clone T59804.

SEQ ID NO: 898 represents the cDNA sequence for clone T59806.

SEQ ID NO: 899 represents the cDNA sequence for clone T59849.

SEQ ID NO: 900 represents the cDNA sequence for clone T59850.

SEQ ID NO: 901 represents the cDNA sequence for clone T59853.

SEQ ID NO: 902 represents the cDNA sequence for clone T59856.

SEQ ID NO: 903 represents the cDNA sequence for clone T59858.

SEQ ID NO: 904 represents the cDNA sequence for clone T59866.

SEQ ID NO: 905 represents the cDNA sequence for clone T59871.

SEQ ID NO: 906 represents the cDNA sequence for clone T59881.

SEQ ID NO: 907 represents the cDNA sequence for clone T59887.

SEQ ID NO: 908 represents the cDNA sequence for clone T59888.

SEQ ID NO: 909 represents the cDNA sequence for clone T59896.

SEQ ID NO: 910 represents the cDNA sequence for clone T59897.

SEQ ID NO: 911 represents the cDNA sequence for clone T59898.

SEQ ID NO: 912 represents the cDNA sequence for clone T59901.

SEQ ID NO: 913 represents the cDNA sequence for clone T59906.

SEQ ID NO: 914 represents the cDNA sequence for clone T59909.

SEQ ID NO: 915 represents the cDNA sequence for clone T59912.

SEQ ID NO: 916 represents the cDNA sequence for clone T59913.

SEQ ID NO: 917 represents the cDNA sequence for clone T59914.

SEQ ID NO: 918 represents the cDNA sequence for clone T59922.

SEQ ID NO: 919 represents the cDNA sequence for clone T59928.

SEQ ID NO: 920 represents the cDNA sequence for clone T59972.

SEQ ID NO: 921 represents the cDNA sequence for clone T59976.

SEQ ID NO: 922 represents the cDNA sequence for clone T59979.

SEQ ID NO: 923 represents the cDNA sequence for clone T59980.

SEQ ID NO: 924 represents the cDNA sequence for clone T59987.

SEQ ID NO: 925 represents the cDNA sequence for clone T59988.

SEQ ID NO: 926 represents the cDNA sequence for clone T59991.

SEQ ID NO: 927 represents the cDNA sequence for clone T59992.

SEQ ID NO: 928 represents the cDNA sequence for clone T59993.

SEQ ID NO: 929 represents the cDNA sequence for clone T60078.

SEQ ID NO: 930 represents the cDNA sequence for clone T60079.

SEQ ID NO: 931 represents the cDNA sequence for clone T60081.

SEQ ID NO: 932 represents the cDNA sequence for clone T60083.

SEQ ID NO: 933 represents the cDNA sequence for clone T60084.

SEQ ID NO: 934 represents the cDNA sequence for clone T60090.

SEQ ID NO: 935 represents the cDNA sequence for clone T60092.

SEQ ID NO: 936 represents the cDNA sequence for clone T60093.

SEQ ID NO: 937 represents the cDNA sequence for clone T60115.

SEQ ID NO: 938 represents the cDNA sequence for clone T60120.

SEQ ID NO: 939 represents the cDNA sequence for clone T60121.

SEQ ID NO: 940 represents the cDNA sequence for clone T60123.

SEQ ID NO: 941 represents the cDNA sequence for clone T60126.

SEQ ID NO: 942 represents the cDNA sequence for clone T60171.

SEQ ID NO: 943 represents the cDNA sequence for clone T60172.

SEQ ID NO: 944 represents the cDNA sequence for clone T60175.

SEQ ID NO: 945 represents the cDNA sequence for clone T60176.

SEQ ID NO: 946 represents the cDNA sequence for clone T60177.

SEQ ID NO: 947 represents the cDNA sequence for clone T60178.

SEQ ID NO: 948 represents the cDNA sequence for clone T60182.

SEQ ID NO: 949 represents the cDNA sequence for clone T60183.

SEQ ID NO: 950 represents the cDNA sequence for clone T60184.

SEQ ID NO: 951 represents the cDNA sequence for clone T60185.

SEQ ID NO: 952 represents the cDNA sequence for clone T60233.

SEQ ID NO: 953 represents the cDNA sequence for clone T60238.

SEQ ID NO: 954 represents the cDNA sequence for clone T60239.

SEQ ID NO: 955 represents the cDNA sequence for clone T60241.

SEQ ID NO: 956 represents the cDNA sequence for clone T60244.

SEQ ID NO: 957 represents the cDNA sequence for clone T60680.

SEQ ID NO: 958 represents the cDNA sequence for clone T60687.

SEQ ID NO: 959 represents the cDNA sequence for clone T60688.

SEQ ID NO: 960 represents the cDNA sequence for clone T60689.

SEQ ID NO: 961 represents the cDNA sequence for clone T60694.

SEQ ID NO: 962 represents the cDNA sequence for clone T60696.

SEQ ID NO: 963 represents the cDNA sequence for clone T60700.

SEQ ID NO: 964 represents the cDNA sequence for clone T60704.

SEQ ID NO: 965 represents the cDNA sequence for clone T60706.

SEQ ID NO: 966 represents the cDNA sequence for clone T60707.

SEQ ID NO: 967 represents the cDNA sequence for clone T60708.

SEQ ID NO: 968 represents the cDNA sequence for clone T60716.

SEQ ID NO: 969 represents the cDNA sequence for clone T60717.

SEQ ID NO: 970 represents the cDNA sequence for clone T60721.

SEQ ID NO: 971 represents the cDNA sequence for clone T60727.

SEQ ID NO: 972 represents the cDNA sequence for clone T60730.

SEQ ID NO: 973 represents the cDNA sequence for clone T60731.

SEQ ID NO: 974 represents the cDNA sequence for clone T60732.

SEQ ID NO: 975 represents the cDNA sequence for clone T60733.

SEQ ID NO: 976 represents the cDNA sequence for clone T60744.

SEQ ID NO: 977 represents the cDNA sequence for clone T60748.

SEQ ID NO: 978 represents the cDNA sequence for clone T60749.

SEQ ID NO: 979 represents the cDNA sequence for clone T60750.

SEQ ID NO: 980 represents the cDNA sequence for clone T60751.

SEQ ID NO: 981 represents the cDNA sequence for clone T60758.

SEQ ID NO: 982 represents the cDNA sequence for clone T60759.

SEQ ID NO: 983 represents the cDNA sequence for clone T60762.

SEQ ID NO: 984 represents the cDNA sequence for clone T60765.

SEQ ID NO: 985 represents the cDNA sequence for clone T60771.

SEQ ID NO: 986 represents the cDNA sequence for clone T60777.

SEQ ID NO: 987 represents the cDNA sequence for clone T60779.

SEQ ID NO: 988 represents the cDNA sequence for clone T60781.

SEQ ID NO: 989 represents the cDNA sequence for clone T60783.

SEQ ID NO: 990 represents the cDNA sequence for clone T60786.

SEQ ID NO: 991 represents the cDNA sequence for clone T60788.

SEQ ID NO: 992 represents the cDNA sequence for clone T60789.

SEQ ID NO: 993 represents the cDNA sequence for clone T60791.

SEQ ID NO: 994 represents the cDNA sequence for clone T60796.

SEQ ID NO: 995 represents the cDNA sequence for clone T60797.

SEQ ID NO: 996 represents the cDNA sequence for clone T60800.

SEQ ID NO: 997 represents the cDNA sequence for clone T60801.

SEQ ID NO: 998 represents the cDNA sequence for clone T60804.

SEQ ID NO: 999 represents the cDNA sequence for clone T60806.

SEQ ID NO: 1000 represents the cDNA sequence for clone T60812.

SEQ ID NO: 1001 represents the cDNA sequence for clone T60815.

SEQ ID NO: 1002 represents the cDNA sequence for clone T60816.

SEQ ID NO: 1003 represents the cDNA sequence for clone T60817.

SEQ ID NO: 1004 represents the cDNA sequence for clone T60818.

SEQ ID NO: 1005 represents the cDNA sequence for clone T60820.

SEQ ID NO: 1006 represents the cDNA sequence for clone T60822.

SEQ ID NO: 1007 represents the cDNA sequence for clone T60827.

SEQ ID NO: 1008 represents the cDNA sequence for clone T60829.

SEQ ID NO: 1009 represents the cDNA sequence for clone T60832.

SEQ ID NO: 1010 represents the cDNA sequence for clone T60834.

SEQ ID NO: 1011 represents the cDNA sequence for clone T60840.

SEQ ID NO: 1012 represents the cDNA sequence for clone T60843.

SEQ ID NO: 1013 represents the cDNA sequence for clone T60847.

SEQ ID NO: 1014 represents the cDNA sequence for clone T60848.

SEQ ID NO: 1015 represents the cDNA sequence for clone T60856.

SEQ ID NO: 1016 represents the cDNA sequence for clone T60858.

SEQ ID NO: 1017 represents the cDNA sequence for clone T60859.

SEQ ID NO: 1018 represents the cDNA sequence for clone T60860.

SEQ ID NO: 1019 represents the cDNA sequence for clone T60861.

SEQ ID NO: 1020 represents the cDNA sequence for clone T60862.

SEQ ID NO: 1021 represents the cDNA sequence for clone T60863.

SEQ ID NO: 1022 represents the cDNA sequence for clone T60864.

SEQ ID NO: 1023 represents the cDNA sequence for clone T60873.

SEQ ID NO: 1024 represents the cDNA sequence for clone T60874.

SEQ ID NO: 1025 represents the cDNA sequence for clone T60875.

SEQ ID NO: 1026 represents the cDNA sequence for clone T60885.

SEQ ID NO: 1027 represents the cDNA sequence for clone T60886.

SEQ ID NO: 1028 represents the cDNA sequence for clone T60887.

SEQ ID NO: 1029 represents the cDNA sequence for clone T60889.

SEQ ID NO: 1030 represents the cDNA sequence for clone T60891.

SEQ ID NO: 1031 represents the cDNA sequence for clone T60892.

SEQ ID NO: 1032 represents the cDNA sequence for clone T60893.

SEQ ID NO: 1033 represents the cDNA sequence for clone T60894.

SEQ ID NO: 1034 represents the cDNA sequence for clone T60896.

SEQ ID NO: 1035 represents the cDNA sequence for clone T60899.

SEQ ID NO: 1036 represents the cDNA sequence for clone T60903.

SEQ ID NO: 1037 represents the cDNA sequence for clone T61014.

SEQ ID NO: 1038 represents the cDNA sequence for clone T61016.

SEQ ID NO: 1039 represents the cDNA sequence for clone T61024.

SEQ ID NO: 1040 represents the cDNA sequence for clone T61026.

SEQ ID NO: 1041 represents the cDNA sequence for clone T61027.

SEQ ID NO: 1042 represents the cDNA sequence for clone T61030.

SEQ ID NO: 1043 represents the cDNA sequence for clone T61031.

SEQ ID NO: 1044 represents the cDNA sequence for clone T61033.

SEQ ID NO: 1045 represents the cDNA sequence for clone T61035.

SEQ ID NO: 1046 represents the cDNA sequence for clone T61036.

SEQ ID NO: 1047 represents the cDNA sequence for clone T61042.

SEQ ID NO: 1048 represents the cDNA sequence for clone T61043.

SEQ ID NO: 1049 represents the cDNA sequence for clone T61044.

SEQ ID NO: 1050 represents the cDNA sequence for clone T61047.

SEQ ID NO: 1051 represents the cDNA sequence for clone T61049.

SEQ ID NO: 1052 represents the cDNA sequence for clone T61050.

SEQ ID NO: 1053 represents the cDNA sequence for clone T61051.

SEQ ID NO: 1054 represents the cDNA sequence for clone T61053.

SEQ ID NO: 1055 represents the cDNA sequence for clone T61055.

SEQ ID NO: 1056 represents the cDNA sequence for clone T61057.

SEQ ID NO: 1057 represents the cDNA sequence for clone T61058.

SEQ ID NO: 1058 represents the cDNA sequence for clone T61063.

SEQ ID NO: 1059 represents the cDNA sequence for clone T61097.

SEQ ID NO: 1060 represents the cDNA sequence for clone T61099.

SEQ ID NO: 1061 represents the cDNA sequence for clone T61139.

SEQ ID NO: 1062 represents the cDNA sequence for clone T61141.

SEQ ID NO: 1063 represents the cDNA sequence for clone T61142.

SEQ ID NO: 1064 represents the cDNA sequence for clone T61144.

SEQ ID NO: 1065 represents the cDNA sequence for clone T61147.

SEQ ID NO: 1066 represents the cDNA sequence for clone T61152.

SEQ ID NO: 1067 represents the cDNA sequence for clone T61155.

SEQ ID NO: 1068 represents the cDNA sequence for clone T61157.

SEQ ID NO: 1069 represents the cDNA sequence for clone T61159.

SEQ ID NO: 1070 represents the cDNA sequence for clone T61160.

SEQ ID NO: 1071 represents the cDNA sequence for clone T61162.

SEQ ID NO: 1072 represents the cDNA sequence for clone T61165.

SEQ ID NO: 1073 represents the cDNA sequence for clone T61492.

SEQ ID NO: 1074 represents the cDNA sequence for clone T61493.

SEQ ID NO: 1075 represents the cDNA sequence for clone T61496.

SEQ ID NO: 1076 represents the cDNA sequence for clone T61501.

SEQ ID NO: 1077 represents the cDNA sequence for clone T61502.

SEQ ID NO: 1078 represents the cDNA sequence for clone T61503.

SEQ ID NO: 1079 represents the cDNA sequence for clone T61504.

SEQ ID NO: 1080 represents the cDNA sequence for clone T61505.

SEQ ID NO: 1081 represents the cDNA sequence for clone T61507.

SEQ ID NO: 1082 represents the cDNA sequence for clone T61511.

SEQ ID NO: 1083 represents the cDNA sequence for clone T61512.

SEQ ID NO: 1084 represents the cDNA sequence for clone T61515.

SEQ ID NO: 1085 represents the cDNA sequence for clone T61518.

SEQ ID NO: 1086 represents the cDNA sequence for clone T61519.

SEQ ID NO: 1087 represents the cDNA sequence for clone T61521.

SEQ ID NO: 1088 represents the cDNA sequence for clone T61525.

SEQ ID NO: 1089 represents the cDNA sequence for clone T61526.

SEQ ID NO: 1090 represents the cDNA sequence for clone T61527.

SEQ ID NO: 1091 represents the cDNA sequence for clone T61530.

SEQ ID NO: 1092 represents the cDNA sequence for clone T61536.

SEQ ID NO: 1093 represents the cDNA sequence for clone T61540.

SEQ ID NO: 1094 represents the cDNA sequence for clone T61542.

SEQ ID NO: 1095 represents the cDNA sequence for clone T61544.

SEQ ID NO: 1096 represents the cDNA sequence for clone T61545.

SEQ ID NO: 1097 represents the cDNA sequence for clone T61548.

SEQ ID NO: 1098 represents the cDNA sequence for clone T61549.

SEQ ID NO: 1099 represents the cDNA sequence for clone T61550.

SEQ ID NO: 1100 represents the cDNA sequence for clone T61551.

SEQ ID NO: 1101 represents the cDNA sequence for clone T61555.

SEQ ID NO: 1102 represents the cDNA sequence for clone T61556.

SEQ ID NO: 1103 represents the cDNA sequence for clone T61560.

SEQ ID NO: 1104 represents the cDNA sequence for clone T61563.

SEQ ID NO: 1105 represents the cDNA sequence for clone T61565.

SEQ ID NO: 1106 represents the cDNA sequence for clone T61570.

SEQ ID NO: 1107 represents the cDNA sequence for clone T61572.

SEQ ID NO: 1108 represents the cDNA sequence for clone T61573.

SEQ ID NO: 1109 represents the cDNA sequence for clone T61576.

SEQ ID NO: 1110 represents the cDNA sequence for clone T61578.

SEQ ID NO: 1111 represents the cDNA sequence for clone T61579.

SEQ ID NO: 1112 represents the cDNA sequence for clone T61582.

SEQ ID NO: 1113 represents the cDNA sequence for clone T61583.

SEQ ID NO: 1114 represents the cDNA sequence for clone T61584.

SEQ ID NO: 1115 represents the cDNA sequence for clone T61587.

SEQ ID NO: 1116 represents the cDNA sequence for clone T61593.

SEQ ID NO: 1117 represents the cDNA sequence for clone T61594.

SEQ ID NO: 1118 represents the cDNA sequence for clone T61598.

SEQ ID NO: 1119 represents the cDNA sequence for clone R95983.

SEQ ID NO: 1120 represents the cDNA sequence for clone R96098.

SEQ ID NO: 1121 represents the cDNA sequence for clone H52225.

SEQ ID NO: 1122 represents the cDNA sequence for clone H52226.

SEQ ID NO: 1123 represents the cDNA sequence for clone H52235.

SEQ ID NO: 1124 represents the cDNA sequence for clone H52239.

SEQ ID NO: 1125 represents the cDNA sequence for clone H52240.

SEQ ID NO: 1126 represents the cDNA sequence for clone H52253.

SEQ ID NO: 1127 represents the cDNA sequence for clone H52258.

SEQ ID NO: 1128 represents the cDNA sequence for clone H52261.

SEQ ID NO: 1129 represents the cDNA sequence for clone H52314.

SEQ ID NO: 1130 represents the cDNA sequence for clone H52316.

SEQ ID NO: 1131 represents the cDNA sequence for clone H52317.

SEQ ID NO: 1132 represents the cDNA sequence for clone H52319.

SEQ ID NO: 1133 represents the cDNA sequence for clone H52323.

SEQ ID NO: 1134 represents the cDNA sequence for clone H52326.

SEQ ID NO: 1135 represents the cDNA sequence for clone H52331.

SEQ ID NO: 1136 represents the cDNA sequence for clone H52337.

SEQ ID NO: 1137 represents the cDNA sequence for clone H52344.

SEQ ID NO: 1138 represents the cDNA sequence for clone H52351.

SEQ ID NO: 1139 represents the cDNA sequence for clone H52353.

SEQ ID NO: 1140 represents the cDNA sequence for clone H52354.

SEQ ID NO: 1141 represents the cDNA sequence for clone H52356.

SEQ ID NO: 1142 represents the cDNA sequence for clone H52358.

SEQ ID NO: 1143 represents the cDNA sequence for clone H52649.

SEQ ID NO: 1144 represents the cDNA sequence for clone H52650.

SEQ ID NO: 1145 represents the cDNA sequence for clone H52654.

SEQ ID NO: 1146 represents the cDNA sequence for clone H52667.

SEQ ID NO: 1147 represents the cDNA sequence for clone H52672.

SEQ ID NO: 1148 represents the cDNA sequence for clone H52681.

SEQ ID NO: 1149 represents the cDNA sequence for clone H52683.

SEQ ID NO: 1150 represents the cDNA sequence for clone H53619.

SEQ ID NO: 1151 represents the cDNA sequence for clone H53632.

SEQ ID NO: 1152 represents the cDNA sequence for clone H53634.

SEQ ID NO: 1153 represents the cDNA sequence for clone H53651.

SEQ ID NO: 1154 represents the cDNA sequence for clone H53674.

SEQ ID NO: 1155 represents the cDNA sequence for clone H53676.

SEQ ID NO: 1156 represents the cDNA sequence for clone H53686.

SEQ ID NO: 1157 represents the cDNA sequence for clone H53687.

SEQ ID NO: 1158 represents the cDNA sequence for clone H53691.

SEQ ID NO: 1159 represents the cDNA sequence for clone H53694.

SEQ ID NO: 1160 represents the cDNA sequence for clone H53701.

SEQ ID NO: 1161 represents the cDNA sequence for clone H53702.

SEQ ID NO: 1162 represents the cDNA sequence for clone H53703.

SEQ ID NO: 1163 represents the cDNA sequence for clone H53712.

SEQ ID NO: 1164 represents the cDNA sequence for clone H53718.

SEQ ID NO: 1165 represents the cDNA sequence for clone H53722.

SEQ ID NO: 1166 represents the cDNA sequence for clone H53723.

SEQ ID NO: 1167 represents the cDNA sequence for clone H53727.

SEQ ID NO: 1168 represents the cDNA sequence for clone H53728.

SEQ ID NO: 1169 represents the cDNA sequence for clone H53730.

SEQ ID NO: 1170 represents the cDNA sequence for clone H53731.

SEQ ID NO: 1171 represents the cDNA sequence for clone H53736.

SEQ ID NO: 1172 represents the cDNA sequence for clone H53737.

SEQ ID NO: 1173 represents the cDNA sequence for clone H53739.

SEQ ID NO: 1174 represents the cDNA sequence for clone H61135.

SEQ ID NO: 1175 represents the cDNA sequence for clone H61142.

SEQ ID NO: 1176 represents the cDNA sequence for clone H61144.

SEQ ID NO: 1177 represents the cDNA sequence for clone H61145.

SEQ ID NO: 1178 represents the cDNA sequence for clone H61150.

SEQ ID NO: 1179 represents the cDNA sequence for clone H61155.

SEQ ID NO: 1180 represents the cDNA sequence for clone H61158.

SEQ ID NO: 1181 represents the cDNA sequence for clone H61161.

SEQ ID NO: 1182 represents the cDNA sequence for clone H61170.

SEQ ID NO: 1183 represents the cDNA sequence for clone H61171.

SEQ ID NO: 1184 represents the cDNA sequence for clone H61179.

SEQ ID NO: 1185 represents the cDNA sequence for clone H61192.

SEQ ID NO: 1186 represents the cDNA sequence for clone H61201.

SEQ ID NO: 1187 represents the cDNA sequence for clone H61206.

SEQ ID NO: 1188 represents the cDNA sequence for clone H61222.

SEQ ID NO: 1189 represents the cDNA sequence for clone H61223.

SEQ ID NO: 1190 represents the cDNA sequence for clone H61225.

SEQ ID NO: 1191 represents the cDNA sequence for clone H61241.

SEQ ID NO: 1192 represents the cDNA sequence for clone H61255.

SEQ ID NO: 1193 represents the cDNA sequence for clone H61259.

SEQ ID NO: 1194 represents the cDNA sequence for clone H61261.

SEQ ID NO: 1195 represents the cDNA sequence for clone H61262.

SEQ ID NO: 1196 represents the cDNA sequence for clone H61263.

SEQ ID NO: 1197 represents the cDNA sequence for clone H61266.

SEQ ID NO: 1198 represents the cDNA sequence for clone H61269.

SEQ ID NO: 1199 represents the cDNA sequence for clone H61271.

SEQ ID NO: 1200 represents the cDNA sequence for clone H61276.

SEQ ID NO: 1201 represents the cDNA sequence for clone H61279.

SEQ ID NO: 1202 represents the cDNA sequence for clone H61280.

SEQ ID NO: 1203 represents the cDNA sequence for clone H61282.

SEQ ID NO: 1204 represents the cDNA sequence for clone H61285.

SEQ ID NO: 1205 represents the cDNA sequence for clone H61294.

SEQ ID NO: 1206 represents the cDNA sequence for clone H61302.

SEQ ID NO: 1207 represents the cDNA sequence for clone H61303.

SEQ ID NO: 1208 represents the cDNA sequence for clone H61305.

SEQ ID NO: 1209 represents the cDNA sequence for clone H61312.

SEQ ID NO: 1210 represents the cDNA sequence for clone H61313.

SEQ ID NO: 1211 represents the cDNA sequence for clone H61320.

SEQ ID NO: 1212 represents the cDNA sequence for clone H61322.

SEQ ID NO: 1213 represents the cDNA sequence for clone H61324.

SEQ ID NO: 1214 represents the cDNA sequence for clone H61325.

SEQ ID NO: 1215 represents the cDNA sequence for clone H61327.

SEQ ID NO: 1216 represents the cDNA sequence for clone H61328.

SEQ ID NO: 1217 represents the cDNA sequence for clone H61337.

SEQ ID NO: 1218 represents the cDNA sequence for clone H61342.

SEQ ID NO: 1219 represents the cDNA sequence for clone H61343.

SEQ ID NO: 1220 represents the cDNA sequence for clone H61351.

SEQ ID NO: 1221 represents the cDNA sequence for clone H61356.

SEQ ID NO: 1222 represents the cDNA sequence for clone H61357.

SEQ ID NO: 1223 represents the cDNA sequence for clone H61358.

SEQ ID NO: 1224 represents the cDNA sequence for clone H61360.

SEQ ID NO: 1225 represents the cDNA sequence for clone H61374.

SEQ ID NO: 1226 represents the cDNA sequence for clone H61379.

SEQ ID NO: 1227 represents the cDNA sequence for clone H61383.

SEQ ID NO: 1228 represents the cDNA sequence for clone H61384.

SEQ ID NO: 1229 represents the cDNA sequence for clone H61385.

SEQ ID NO: 1230 represents the cDNA sequence for clone H61386.

SEQ ID NO: 1231 represents the cDNA sequence for clone H61387.

SEQ ID NO: 1232 represents the cDNA sequence for clone H61390.

SEQ ID NO: 1233 represents the cDNA sequence for clone H61398.

SEQ ID NO: 1234 represents the cDNA sequence for clone H61404.

SEQ ID NO: 1235 represents the cDNA sequence for clone H61753.

SEQ ID NO: 1236 represents the cDNA sequence for clone H61758.

SEQ ID NO: 1237 represents the cDNA sequence for clone H61768.

SEQ ID NO: 1238 represents the cDNA sequence for clone H61773.

SEQ ID NO: 1239 represents the cDNA sequence for clone H61775.

SEQ ID NO: 1240 represents the cDNA sequence for clone H61776.

SEQ ID NO: 1241 represents the cDNA sequence for clone H61783.

SEQ ID NO: 1242 represents the cDNA sequence for clone H61784.

SEQ ID NO: 1243 represents the cDNA sequence for clone H61796.

SEQ ID NO: 1244 represents the cDNA sequence for clone H61797.

SEQ ID NO: 1245 represents the cDNA sequence for clone H61799.

SEQ ID NO: 1246 represents the cDNA sequence for clone H61810.

SEQ ID NO: 1247 represents the cDNA sequence for clone H61811.

SEQ ID NO: 1248 represents the cDNA sequence for clone H61814.

SEQ ID NO: 1249 represents the cDNA sequence for clone H61820.

SEQ ID NO: 1250 represents the cDNA sequence for clone H61824.

SEQ ID NO: 1251 represents the cDNA sequence for clone H61830.

SEQ ID NO: 1252 represents the cDNA sequence for clone H61831.

SEQ ID NO: 1253 represents the cDNA sequence for clone H61832.

SEQ ID NO: 1254 represents the cDNA sequence for clone H61838.

SEQ ID NO: 1255 represents the cDNA sequence for clone H62048.

SEQ ID NO: 1256 represents the cDNA sequence for clone H62052.

SEQ ID NO: 1257 represents the cDNA sequence for clone H62057.

SEQ ID NO: 1258 represents the cDNA sequence for clone H62064.

SEQ ID NO: 1259 represents the cDNA sequence for clone H62068.

SEQ ID NO: 1260 represents the cDNA sequence for clone H62073.

SEQ ID NO: 1261 represents the cDNA sequence for clone H62074.

SEQ ID NO: 1262 represents the cDNA sequence for clone H62079.

SEQ ID NO: 1263 represents the cDNA sequence for clone H62097.

SEQ ID NO: 1264 represents the cDNA sequence for clone H62105.

SEQ ID NO: 1265 represents the cDNA sequence for clone H62107.

SEQ ID NO: 1266 represents the cDNA sequence for clone H62111.

SEQ ID NO: 1267 represents the cDNA sequence for clone H62113.

SEQ ID NO: 1268 represents the cDNA sequence for clone H62116.

SEQ ID NO: 1269 represents the cDNA sequence for clone H62343.

SEQ ID NO: 1270 represents the cDNA sequence for clone H62344.

SEQ ID NO: 1271 represents the cDNA sequence for clone H62351.

SEQ ID NO: 1272 represents the cDNA sequence for clone H62352.

SEQ ID NO: 1273 represents the cDNA sequence for clone H62358.

SEQ ID NO: 1274 represents the cDNA sequence for clone H62362.

SEQ ID NO: 1275 represents the cDNA sequence for clone H62383.

SEQ ID NO: 1276 represents the cDNA sequence for clone H62385.

SEQ ID NO: 1277 represents the cDNA sequence for clone H62396.

SEQ ID NO: 1278 represents the cDNA sequence for clone H62400.

SEQ ID NO: 1279 represents the cDNA sequence for clone H62404.

SEQ ID NO: 1280 represents the cDNA sequence for clone H62405.

SEQ ID NO: 1281 represents the cDNA sequence for clone H62421.

SEQ ID NO: 1282 represents the cDNA sequence for clone AA074711.

SEQ ID NO: 1283 represents the cDNA sequence for clone AA074714.

SEQ ID NO: 1284 represents the cDNA sequence for clone AA074721.

SEQ ID NO: 1285 represents the cDNA sequence for clone AA074590.

SEQ ID NO: 1286 represents the cDNA sequence for clone AA074591.

SEQ ID NO: 1287 represents the cDNA sequence for clone AA074677.

SEQ ID NO: 1288 represents the cDNA sequence for clone AA074665.

SEQ ID NO: 1289 represents the cDNA sequence for clone AA074668.

SEQ ID NO: 1290 represents the cDNA sequence for clone AA074732.

SEQ ID NO: 1291 represents the cDNA sequence for clone AA074737.

SEQ ID NO: 1292 represents the cDNA sequence for clone AA074751.

SEQ ID NO: 1293 represents the cDNA sequence for clone AA074763.

SEQ ID NO: 1294 represents the cDNA sequence for clone AA074765.

SEQ ID NO: 1295 represents the cDNA sequence for clone AA074767.

SEQ ID NO: 1296 represents the cDNA sequence for clone AA074768.

SEQ ID NO: 1297 represents the cDNA sequence for clone AA074771.

SEQ ID NO: 1298 represents the cDNA sequence for clone AA074774.

SEQ ID NO: 1299 represents the cDNA sequence for clone AA074778.

SEQ ID NO: 1300 represents the cDNA sequence for clone AA074789.

SEQ ID NO: 1301 represents the cDNA sequence for clone AA074790.

SEQ ID NO: 1302 represents the cDNA sequence for clone AA074610.

SEQ ID NO: 1303 represents the cDNA sequence for clone AA074612.

SEQ ID NO: 1304 represents the cDNA sequence for clone AA074793.

SEQ ID NO: 1305 represents the cDNA sequence for clone AA074794.

SEQ ID NO: 1306 represents the cDNA sequence for clone AA074818.

SEQ ID NO: 1307 represents the cDNA sequence for clone AA074822.

SEQ ID NO: 1308 represents the cDNA sequence for clone AA074801.

SEQ ID NO: 1309 represents the cDNA sequence for clone AA074803.

SEQ ID NO: 1310 represents the cDNA sequence for clone AA074846.

SEQ ID NO: 1311 represents the cDNA sequence for clone AA074809.

SEQ ID NO: 1312 represents the cDNA sequence for clone AA074812.

SEQ ID NO: 1313 represents the cDNA sequence for clone AA074813.

SEQ ID NO: 1314 represents the cDNA sequence for clone AA074814.

SEQ ID NO: 1315 represents the cDNA sequence for clone AA074723.

SEQ ID NO: 1316 represents the cDNA sequence for clone AA074724.

SEQ ID NO: 1317 represents the cDNA sequence for clone AA074851.

SEQ ID NO: 1318 represents the cDNA sequence for clone AA074857.

SEQ ID NO: 1319 represents the cDNA sequence for clone AA074863.

SEQ ID NO: 1320 represents the cDNA sequence for clone AA074867.

SEQ ID NO: 1321 represents the cDNA sequence for clone AA074888.

SEQ ID NO: 1322 represents the cDNA sequence for clone AA074889.

SEQ ID NO: 1323 represents the cDNA sequence for clone AA074871.

SEQ ID NO: 1324 represents the cDNA sequence for clone AA074873.

SEQ ID NO: 1325 represents the cDNA sequence for clone AA074907.

SEQ ID NO: 1326 represents the cDNA sequence for clone AA074912.

SEQ ID NO: 1327 represents the cDNA sequence for clone AA074915.

SEQ ID NO: 1328 represents the cDNA sequence for clone AA074920.

SEQ ID NO: 1329 represents the cDNA sequence for clone AA074923.

SEQ ID NO: 1330 represents the cDNA sequence for clone AA074897.

SEQ ID NO: 1331 represents the cDNA sequence for clone AA074899.

SEQ ID NO: 1332 represents the cDNA sequence for clone AA074904.

SEQ ID NO: 1333 represents the cDNA sequence for clone AA074932.

SEQ ID NO: 1334 represents the cDNA sequence for clone AA074934.

SEQ ID NO: 1335 represents the cDNA sequence for clone AA074935.

SEQ ID NO: 1336 represents the cDNA sequence for clone AA074939.

SEQ ID NO: 1337 represents the cDNA sequence for clone AA074959.

SEQ ID NO: 1338 represents the cDNA sequence for clone AA074971.

SEQ ID NO: 1339 represents the cDNA sequence for clone AA074974.

SEQ ID NO: 1340 represents the cDNA sequence for clone AA074976.

SEQ ID NO: 1341 represents the cDNA sequence for clone AA074981.

SEQ ID NO: 1342 represents the cDNA sequence for clone AA074987.

SEQ ID NO: 1343 represents the cDNA sequence for clone AA074996.

SEQ ID NO: 1344 represents the cDNA sequence for clone AA074997.

SEQ ID NO: 1345 represents the cDNA sequence for clone AA075000.

SEQ ID NO: 1346 represents the cDNA sequence for clone AA074949.

SEQ ID NO: 1347 represents the cDNA sequence for clone AA074950.

SEQ ID NO: 1348 represents the cDNA sequence for clone AA074951.

SEQ ID NO: 1349 represents the cDNA sequence for clone AA074952.

SEQ ID NO: 1350 represents the cDNA sequence for clone AA075007.

SEQ ID NO: 1351 represents the cDNA sequence for clone AA075010.

SEQ ID NO: 1352 represents the cDNA sequence for clone AA075027.

SEQ ID NO: 1353 represents the cDNA sequence for clone AA075030.

SEQ ID NO: 1354 represents the cDNA sequence for clone AA075031.

SEQ ID NO: 1355 represents the cDNA sequence for clone AA075041.

SEQ ID NO: 1356 represents the cDNA sequence for clone AA075042.

SEQ ID NO: 1357 represents the cDNA sequence for clone AA075047.

SEQ ID NO: 1358 represents the cDNA sequence for clone AA075048.

SEQ ID NO: 1359 represents the cDNA sequence for clone AA075056.

SEQ ID NO: 1360 represents the cDNA sequence for clone AA075058.

SEQ ID NO: 1361 represents the cDNA sequence for clone AA075061.

SEQ ID NO: 1362 represents the cDNA sequence for clone AA075062.

SEQ ID NO: 1363 represents the cDNA sequence for clone AA075064.

SEQ ID NO: 1364 represents the cDNA sequence for clone AA074882.

SEQ ID NO: 1365 represents the cDNA sequence for clone AA075025.

SEQ ID NO: 1366 represents the cDNA sequence for clone AA075013.

SEQ ID NO: 1367 represents the cDNA sequence for clone AA075078.

SEQ ID NO: 1368 represents the cDNA sequence for clone AA075079.

SEQ ID NO: 1369 represents the cDNA sequence for clone AA075082.

SEQ ID NO: 1370 represents the cDNA sequence for clone AA075103.

SEQ ID NO: 1371 represents the cDNA sequence for clone AA075104.

SEQ ID NO: 1372 represents the cDNA sequence for clone AA075105.

SEQ ID NO: 1373 represents the cDNA sequence for clone AA075107.

SEQ ID NO: 1374 represents the cDNA sequence for clone AA075118.

SEQ ID NO: 1375 represents the cDNA sequence for clone AA075120.

SEQ ID NO: 1376 represents the cDNA sequence for clone AA075124.

SEQ ID NO: 1377 represents the cDNA sequence for clone AA075126.

SEQ ID NO: 1378 represents the cDNA sequence for clone AA075128.

SEQ ID NO: 1379 represents the cDNA sequence for clone AA075129.

SEQ ID NO: 1380 represents the cDNA sequence for clone AA075069.

SEQ ID NO: 1381 represents the cDNA sequence for clone AA075070.

SEQ ID NO: 1382 represents the cDNA sequence for clone AA075072.

SEQ ID NO: 1383 represents the cDNA sequence for clone AA075077.

SEQ ID NO: 1384 represents the cDNA sequence for clone AA074943.

SEQ ID NO: 1385 represents the cDNA sequence for clone AA074944.

SEQ ID NO: 1386 represents the cDNA sequence for clone AA075140.

SEQ ID NO: 1387 represents the cDNA sequence for clone AA075145.

SEQ ID NO: 1388 represents the cDNA sequence for clone AA075164.

SEQ ID NO: 1389 represents the cDNA sequence for clone AA075168.

SEQ ID NO: 1390 represents the cDNA sequence for clone AA075169.

SEQ ID NO: 1391 represents the cDNA sequence for clone AA075149.

SEQ ID NO: 1392 represents the cDNA sequence for clone AA075188.

SEQ ID NO: 1393 represents the cDNA sequence for clone AA075198.

SEQ ID NO: 1394 represents the cDNA sequence for clone AA075202.

SEQ ID NO: 1395 represents the cDNA sequence for clone AA075203.

SEQ ID NO: 1396 represents the cDNA sequence for clone AA075204.

SEQ ID NO: 1397 represents the cDNA sequence for clone AA075092.

SEQ ID NO: 1398 represents the cDNA sequence for clone AA075093.

SEQ ID NO: 1399 represents the cDNA sequence for clone AA075173.

SEQ ID NO: 1400 represents the cDNA sequence for clone AA075177.

SEQ ID NO: 1401 represents the cDNA sequence for clone AA075178.

SEQ ID NO: 1402 represents the cDNA sequence for clone AA075208.

SEQ ID NO: 1403 represents the cDNA sequence for clone AA075210.

SEQ ID NO: 1404 represents the cDNA sequence for clone AA075211.

SEQ ID NO: 1405 represents the cDNA sequence for clone AA075213.

SEQ ID NO: 1406 represents the cDNA sequence for clone AA075232.

SEQ ID NO: 1407 represents the cDNA sequence for clone AA075233.

SEQ ID NO: 1408 represents the cDNA sequence for clone AA075234.

SEQ ID NO: 1409 represents the cDNA sequence for clone AA075239.

SEQ ID NO: 1410 represents the cDNA sequence for clone AA075243.

SEQ ID NO: 1411 represents the cDNA sequence for clone AA075245.

SEQ ID NO: 1412 represents the cDNA sequence for clone AA075251.

SEQ ID NO: 1413 represents the cDNA sequence for clone AA075255.

SEQ ID NO: 1414 represents the cDNA sequence for clone AA075259.

SEQ ID NO: 1415 represents the cDNA sequence for clone AA075260.

SEQ ID NO: 1416 represents the cDNA sequence for clone AA075131.

SEQ ID NO: 1417 represents the cDNA sequence for clone AA075132.

SEQ ID NO: 1418 represents the cDNA sequence for clone AA075267.

SEQ ID NO: 1419 represents the cDNA sequence for clone AA075268.

SEQ ID NO: 1420 represents the cDNA sequence for clone AA075299.

SEQ ID NO: 1421 represents the cDNA sequence for clone AA075306.

SEQ ID NO: 1422 represents the cDNA sequence for clone AA075309.

SEQ ID NO: 1423 represents the cDNA sequence for clone AA075310.

SEQ ID NO: 1424 represents the cDNA sequence for clone AA075313.

SEQ ID NO: 1425 represents the cDNA sequence for clone AA075318.

SEQ ID NO: 1426 represents the cDNA sequence for clone AA075324.

SEQ ID NO: 1427 represents the cDNA sequence for clone AA075326.

SEQ ID NO: 1428 represents the cDNA sequence for clone AA075327.

SEQ ID NO: 1429 represents the cDNA sequence for clone AA075363.

SEQ ID NO: 1430 represents the cDNA sequence for clone AA075364.

SEQ ID NO: 1431 represents the cDNA sequence for clone AA075365.

SEQ ID NO: 1432 represents the cDNA sequence for clone AA075366.

SEQ ID NO: 1433 represents the cDNA sequence for clone AA075368.

SEQ ID NO: 1434 represents the cDNA sequence for clone AA075369.

SEQ ID NO: 1435 represents the cDNA sequence for clone AA075370.

SEQ ID NO: 1436 represents the cDNA sequence for clone AA075372.

SEQ ID NO: 1437 represents the cDNA sequence for clone AA075373.

SEQ ID NO: 1438 represents the cDNA sequence for clone AA075374.

SEQ ID NO: 1439 represents the cDNA sequence for clone AA075375.

SEQ ID NO: 1440 represents the cDNA sequence for clone AA075376.

SEQ ID NO: 1441 represents the cDNA sequence for clone AA075395.

SEQ ID NO: 1442 represents the cDNA sequence for clone AA075400.

SEQ ID NO: 1443 represents the cDNA sequence for clone AA075217.

SEQ ID NO: 1444 represents the cDNA sequence for clone AA075222.

SEQ ID NO: 1445 represents the cDNA sequence for clone AA075350.

SEQ ID NO: 1446 represents the cDNA sequence for clone AA075354.

SEQ ID NO: 1447 represents the cDNA sequence for clone AA075406.

SEQ ID NO: 1448 represents the cDNA sequence for clone AA075407.

SEQ ID NO: 1449 represents the cDNA sequence for clone AA075408.

SEQ ID NO: 1450 represents the cDNA sequence for clone AA075430.

SEQ ID NO: 1451 represents the cDNA sequence for clone AA075444.

SEQ ID NO: 1452 represents the cDNA sequence for clone AA075450.

SEQ ID NO: 1453 represents the cDNA sequence for clone AA075453.

SEQ ID NO: 1454 represents the cDNA sequence for clone AA075462.

SEQ ID NO: 1455 represents the cDNA sequence for clone AA075464.

SEQ ID NO: 1456 represents the cDNA sequence for clone AA075466.

SEQ ID NO: 1457 represents the cDNA sequence for clone AA075285.

SEQ ID NO: 1458 represents the cDNA sequence for clone AA075419.

SEQ ID NO: 1459 represents the cDNA sequence for clone AA075420.

SEQ ID NO: 1460 represents the cDNA sequence for clone AA075502.

SEQ ID NO: 1461 represents the cDNA sequence for clone AA075516.

SEQ ID NO: 1462 represents the cDNA sequence for clone AA075517.

SEQ ID NO: 1463 represents the cDNA sequence for clone AA075518.

SEQ ID NO: 1464 represents the cDNA sequence for clone AA075519.

SEQ ID NO: 1465 represents the cDNA sequence for clone AA075522.

SEQ ID NO: 1466 represents the cDNA sequence for clone AA075523.

SEQ ID NO: 1467 represents the cDNA sequence for clone AA075524.

SEQ ID NO: 1468 represents the cDNA sequence for clone AA075533.

SEQ ID NO: 1469 represents the cDNA sequence for clone AA075535.

SEQ ID NO: 1470 represents the cDNA sequence for clone AA075340.

SEQ ID NO: 1471 represents the cDNA sequence for clone AA075487.

SEQ ID NO: 1472 represents the cDNA sequence for clone AA075488.

SEQ ID NO: 1473 represents the cDNA sequence for clone AA075491.

SEQ ID NO: 1474 represents the cDNA sequence for clone AA075492.

SEQ ID NO: 1475 represents the cDNA sequence for clone AA075537.

SEQ ID NO: 1476 represents the cDNA sequence for clone AA075538.

SEQ ID NO: 1477 represents the cDNA sequence for clone AA075539.

SEQ ID NO: 1478 represents the cDNA sequence for clone AA075565.

SEQ ID NO: 1479 represents the cDNA sequence for clone AA075574.

SEQ ID NO: 1480 represents the cDNA sequence for clone AA075575.

SEQ ID NO: 1481 represents the cDNA sequence for clone AA075578.

SEQ ID NO: 1482 represents the cDNA sequence for clone AA075579.

SEQ ID NO: 1483 represents the cDNA sequence for clone AA075590.

SEQ ID NO: 1484 represents the cDNA sequence for clone AA075592.

SEQ ID NO: 1485 represents the cDNA sequence for clone AA075593.

SEQ ID NO: 1486 represents the cDNA sequence for clone AA075602.

SEQ ID NO: 1487 represents the cDNA sequence for clone AA075603.

SEQ ID NO: 1488 represents the cDNA sequence for clone AA075412.

SEQ ID NO: 1489 represents the cDNA sequence for clone AA075413.

SEQ ID NO: 1490 represents the cDNA sequence for clone AA075414.

SEQ ID NO: 1491 represents the cDNA sequence for clone AA075416.

SEQ ID NO: 1492 represents the cDNA sequence for clone AA075417.

SEQ ID NO: 1493 represents the cDNA sequence for clone AA075418.

SEQ ID NO: 1494 represents the cDNA sequence for clone AA075560.

SEQ ID NO: 1495 represents the cDNA sequence for clone AA075611.

SEQ ID NO: 1496 represents the cDNA sequence for clone AA075612.

SEQ ID NO: 1497 represents the cDNA sequence for clone AA075631.

SEQ ID NO: 1498 represents the cDNA sequence for clone AA075632.

SEQ ID NO: 1499 represents the cDNA sequence for clone AA075639.

SEQ ID NO: 1500 represents the cDNA sequence for clone AA075640.

SEQ ID NO: 1501 represents the cDNA sequence for clone AA075651.

SEQ ID NO: 1502 represents the cDNA sequence for clone AA075652.

SEQ ID NO: 1503 represents the cDNA sequence for clone AA075659.

SEQ ID NO: 1504 represents the cDNA sequence for clone AA075660.

SEQ ID NO: 1505 represents the cDNA sequence for clone AA075668.

SEQ ID NO: 1506 represents the cDNA sequence for clone AA075669.

SEQ ID NO: 1507 represents the cDNA sequence for clone AA075670.

SEQ ID NO: 1508 represents the cDNA sequence for clone AA075674.

SEQ ID NO: 1509 represents the cDNA sequence for clone AA075676.

SEQ ID NO: 1510 represents the cDNA sequence for clone AA075677.

SEQ ID NO: 1511 represents the cDNA sequence for clone AA075678.

SEQ ID NO: 1512 represents the cDNA sequence for clone AA075699.

SEQ ID NO: 1513 represents the cDNA sequence for clone AA075703.

SEQ ID NO: 1514 represents the cDNA sequence for clone AA075704.

SEQ ID NO: 1515 represents the cDNA sequence for clone AA075708.

SEQ ID NO: 1516 represents the cDNA sequence for clone AA075710.

SEQ ID NO: 1517 represents the cDNA sequence for clone AA075715.

SEQ ID NO: 1518 represents the cDNA sequence for clone AA075720.

SEQ ID NO: 1519 represents the cDNA sequence for clone AA075730.

SEQ ID NO: 1520 represents the cDNA sequence for clone AA075731.

SEQ ID NO: 1521 represents the cDNA sequence for clone AA075732.

SEQ ID NO: 1522 represents the cDNA sequence for clone AA075738.

SEQ ID NO: 1523 represents the cDNA sequence for clone AA075545.

SEQ ID NO: 1524 represents the cDNA sequence for clone AA075547.

SEQ ID NO: 1525 represents the cDNA sequence for clone AA075548.

SEQ ID NO: 1526 represents the cDNA sequence for clone AA075549.

SEQ ID NO: 1527 represents the cDNA sequence for clone AA075689.

SEQ ID NO: 1528 represents the cDNA sequence for clone AA075691.

SEQ ID NO: 1529 represents the cDNA sequence for clone AA075693.

SEQ ID NO: 1530 represents the cDNA sequence for clone AA075694.

SEQ ID NO: 1531 represents the cDNA sequence for clone AA075741.

SEQ ID NO: 1532 represents the cDNA sequence for clone AA075743.

SEQ ID NO: 1533 represents the cDNA sequence for clone AA075747.

SEQ ID NO: 1534 represents the cDNA sequence for clone AA075748.

SEQ ID NO: 1535 represents the cDNA sequence for clone AA075767.

SEQ ID NO: 1536 represents the cDNA sequence for clone AA075770.

SEQ ID NO: 1537 represents the cDNA sequence for clone AA075771.

SEQ ID NO: 1538 represents the cDNA sequence for clone AA075782.

SEQ ID NO: 1539 represents the cDNA sequence for clone AA075783.

SEQ ID NO: 1540 represents the cDNA sequence for clone AA075784.

SEQ ID NO: 1541 represents the cDNA sequence for clone AA075788.

SEQ ID NO: 1542 represents the cDNA sequence for clone AA075994.

SEQ ID NO: 1543 represents the cDNA sequence for clone AA075995.

SEQ ID NO: 1544 represents the cDNA sequence for clone AA076003.

SEQ ID NO: 1545 represents the cDNA sequence for clone AA076004.

SEQ ID NO: 1546 represents the cDNA sequence for clone AA075955.

SEQ ID NO: 1547 represents the cDNA sequence for clone AA075960.

SEQ ID NO: 1548 represents the cDNA sequence for clone AA075961.

SEQ ID NO: 1549 represents the cDNA sequence for clone AA075826.

SEQ ID NO: 1550 represents the cDNA sequence for clone AA075827.

SEQ ID NO: 1551 represents the cDNA sequence for clone AA075828.

SEQ ID NO: 1552 represents the cDNA sequence for clone AA075831.

SEQ ID NO: 1553 represents the cDNA sequence for clone AA075832.

SEQ ID NO: 1554 represents the cDNA sequence for clone AA076005.

SEQ ID NO: 1555 represents the cDNA sequence for clone AA076009.

SEQ ID NO: 1556 represents the cDNA sequence for clone AA076010.

SEQ ID NO: 1557 represents the cDNA sequence for clone AA076021.

SEQ ID NO: 1558 represents the cDNA sequence for clone AA076041.

SEQ ID NO: 1559 represents the cDNA sequence for clone AA076042.

SEQ ID NO: 1560 represents the cDNA sequence for clone AA076045.

SEQ ID NO: 1561 represents the cDNA sequence for clone AA076049.

SEQ ID NO: 1562 represents the cDNA sequence for clone AA076056.

SEQ ID NO: 1563 represents the cDNA sequence for clone AA076061.

SEQ ID NO: 1564 represents the cDNA sequence for clone AA076070.

SEQ ID NO: 1565 represents the cDNA sequence for clone AA076079.

SEQ ID NO: 1566 represents the cDNA sequence for clone AA076081.

SEQ ID NO: 1567 represents the cDNA sequence for clone AA076084.

SEQ ID NO: 1568 represents the cDNA sequence for clone AA075895.

SEQ ID NO: 1569 represents the cDNA sequence for clone AA076139.

SEQ ID NO: 1570 represents the cDNA sequence for clone AA076143.

SEQ ID NO: 1571 represents the cDNA sequence for clone AA076151.

SEQ ID NO: 1572 represents the cDNA sequence for clone AA076154.

SEQ ID NO: 1573 represents the cDNA sequence for clone AA076155.

SEQ ID NO: 1574 represents the cDNA sequence for clone AA076096.

SEQ ID NO: 1575 represents the cDNA sequence for clone AA076098.

SEQ ID NO: 1576 represents the cDNA sequence for clone AA076101.

SEQ ID NO: 1577 represents the cDNA sequence for clone AA076102.

SEQ ID NO: 1578 represents the cDNA sequence for clone AA076027.

SEQ ID NO: 1579 represents the cDNA sequence for clone AA076030.

SEQ ID NO: 1580 represents the cDNA sequence for clone AA076161.

SEQ ID NO: 1581 represents the cDNA sequence for clone AA076162.

SEQ ID NO: 1582 represents the cDNA sequence for clone AA076163.

SEQ ID NO: 1583 represents the cDNA sequence for clone AA076164.

SEQ ID NO: 1584 represents the cDNA sequence for clone AA076165.

SEQ ID NO: 1585 represents the cDNA sequence for clone AA076182.

SEQ ID NO: 1586 represents the cDNA sequence for clone AA076187.

SEQ ID NO: 1587 represents the cDNA sequence for clone AA076228.

SEQ ID NO: 1588 represents the cDNA sequence for clone AA076258.

SEQ ID NO: 1589 represents the cDNA sequence for clone AA076259.

SEQ ID NO: 1590 represents the cDNA sequence for clone AA076264.

SEQ ID NO: 1591 represents the cDNA sequence for clone AA076265.

SEQ ID NO: 1592 represents the cDNA sequence for clone AA076268.

SEQ ID NO: 1593 represents the cDNA sequence for clone AA076269.

SEQ ID NO: 1594 represents the cDNA sequence for clone AA076270.

SEQ ID NO: 1595 represents the cDNA sequence for clone AA076275.

SEQ ID NO: 1596 represents the cDNA sequence for clone AA076277.

SEQ ID NO: 1597 represents the cDNA sequence for clone AA076278.

SEQ ID NO: 1598 represents the cDNA sequence for clone AA076290.

SEQ ID NO: 1599 represents the cDNA sequence for clone AA076349.

SEQ ID NO: 1600 represents the cDNA sequence for clone AA076350.

SEQ ID NO: 1601 represents the cDNA sequence for clone AA076351.

SEQ ID NO: 1602 represents the cDNA sequence for clone AA076356.

SEQ ID NO: 1603 represents the cDNA sequence for clone AA076359.

SEQ ID NO: 1604 represents the cDNA sequence for clone AA076362.

SEQ ID NO: 1605 represents the cDNA sequence for clone AA076365.

SEQ ID NO: 1606 represents the cDNA sequence for clone AA076167.

SEQ ID NO: 1607 represents the cDNA sequence for clone AA076170.

SEQ ID NO: 1608 represents the cDNA sequence for clone AA076172.

SEQ ID NO: 1609 represents the cDNA sequence for clone AA076173.

SEQ ID NO: 1610 represents the cDNA sequence for clone AA076367.

SEQ ID NO: 1611 represents the cDNA sequence for clone AA076373.

SEQ ID NO: 1612 represents the cDNA sequence for clone AA076382.

SEQ ID NO: 1613 represents the cDNA sequence for clone AA076401.

SEQ ID NO: 1614 represents the cDNA sequence for clone AA076402.

SEQ ID NO: 1615 represents the cDNA sequence for clone AA076442.

SEQ ID NO: 1616 represents the cDNA sequence for clone AA076443.

SEQ ID NO: 1617 represents the cDNA sequence for clone AA076462.

SEQ ID NO: 1618 represents the cDNA sequence for clone AA076467.

SEQ ID NO: 1619 represents the cDNA sequence for clone AA076470.

SEQ ID NO: 1620 represents the cDNA sequence for clone AA076472.

SEQ ID NO: 1621 represents the cDNA sequence for clone AA076476.

SEQ ID NO: 1622 represents the cDNA sequence for clone AA076484.

SEQ ID NO: 1623 represents the cDNA sequence for clone AA076485.

SEQ ID NO: 1624 represents the cDNA sequence for clone AA076492.

SEQ ID NO: 1625 represents the cDNA sequence for clone AA076494.

SEQ ID NO: 1626 represents the cDNA sequence for clone AA076496.

SEQ ID NO: 1627 represents the cDNA sequence for clone AA076498.

SEQ ID NO: 1628 represents the cDNA sequence for clone AA076502.

SEQ ID NO: 1629 represents the cDNA sequence for clone AA076311.

SEQ ID NO: 1630 represents the cDNA sequence for clone AA076313.

SEQ ID NO: 1631 represents the cDNA sequence for clone AA076453.

SEQ ID NO: 1632 represents the cDNA sequence for clone AA076457.

SEQ ID NO: 1633 represents the cDNA sequence for clone AA076458.

SEQ ID NO: 1634 represents the cDNA sequence for clone AA076529.

SEQ ID NO: 1635 represents the cDNA sequence for clone AA076392.

SEQ ID NO: 1636 represents the cDNA sequence for clone AA076448.

SEQ ID NO: 1637 represents the cDNA sequence for clone AA076449.

SEQ ID NO: 1638 represents the cDNA sequence for clone AA076450.

SEQ ID NO: 1639 represents the cDNA sequence for clone AA076451.

SEQ ID NO: 1640 represents the cDNA sequence for clone AA076571.

SEQ ID NO: 1641 represents the cDNA sequence for clone AA076581.

SEQ ID NO: 1642 represents the cDNA sequence for clone AA076582.

SEQ ID NO: 1643 represents the cDNA sequence for clone AA076589.

SEQ ID NO: 1644 represents the cDNA sequence for clone AA076591.

SEQ ID NO: 1645 represents the cDNA sequence for clone AA076592.

SEQ ID NO: 1646 represents the cDNA sequence for clone AA076593.

SEQ ID NO: 1647 represents the cDNA sequence for clone AA076594.

SEQ ID NO: 1648 represents the cDNA sequence for clone AA078957.

SEQ ID NO: 1649 represents the cDNA sequence for clone AA078966.

SEQ ID NO: 1650 represents the cDNA sequence for clone AA078970.

SEQ ID NO: 1651 represents the cDNA sequence for clone AA078980.

SEQ ID NO: 1652 represents the cDNA sequence for clone AA078981.

SEQ ID NO: 1653 represents the cDNA sequence for clone AA078983.

SEQ ID NO: 1654 represents the cDNA sequence for clone AA078984.

SEQ ID NO: 1655 represents the cDNA sequence for clone AA078986.

SEQ ID NO: 1656 represents the cDNA sequence for clone AA078799.

SEQ ID NO: 1657 represents the cDNA sequence for clone AA079038.

SEQ ID NO: 1658 represents the cDNA sequence for clone AA079045.

SEQ ID NO: 1659 represents the cDNA sequence for clone AA079048.

SEQ ID NO: 1660 represents the cDNA sequence for clone AA079050.

SEQ ID NO: 1661 represents the cDNA sequence for clone AA078859.

SEQ ID NO: 1662 represents the cDNA sequence for clone AA078864.

SEQ ID NO: 1663 represents the cDNA sequence for clone AA079009.

SEQ ID NO: 1664 represents the cDNA sequence for clone AA079011.

SEQ ID NO: 1665 represents the cDNA sequence for clone AA079063.

SEQ ID NO: 1666 represents the cDNA sequence for clone AA079064.

SEQ ID NO: 1667 represents the cDNA sequence for clone AA079084.

SEQ ID NO: 1668 represents the cDNA sequence for clone AA079085.

SEQ ID NO: 1669 represents the cDNA sequence for clone AA079087.

SEQ ID NO: 1670 represents the cDNA sequence for clone AA079095.

SEQ ID NO: 1671 represents the cDNA sequence for clone AA079096.

SEQ ID NO: 1672 represents the cDNA sequence for clone AA079097.

SEQ ID NO: 1673 represents the cDNA sequence for clone AA079098.

SEQ ID NO: 1674 represents the cDNA sequence for clone AA079127.

SEQ ID NO: 1675 represents the cDNA sequence for clone AA079128.

SEQ ID NO: 1676 represents the cDNA sequence for clone AA079160.

SEQ ID NO: 1677 represents the cDNA sequence for clone AA079161.

SEQ ID NO: 1678 represents the cDNA sequence for clone AA079165.

SEQ ID NO: 1679 represents the cDNA sequence for clone AA079166.

SEQ ID NO: 1680 represents the cDNA sequence for clone AA079177.

SEQ ID NO: 1681 represents the cDNA sequence for clone AA079178.

SEQ ID NO: 1682 represents the cDNA sequence for clone AA079179.

SEQ ID NO: 1683 represents the cDNA sequence for clone AA079180.

SEQ ID NO: 1684 represents the cDNA sequence for clone AA079183.

SEQ ID NO: 1685 represents the cDNA sequence for clone AA079184.

SEQ ID NO: 1686 represents the cDNA sequence for clone AA079189.

SEQ ID NO: 1687 represents the cDNA sequence for clone AA079192.

SEQ ID NO: 1688 represents the cDNA sequence for clone AA079193.

SEQ ID NO: 1689 represents the cDNA sequence for clone AA079649.

SEQ ID NO: 1690 represents the cDNA sequence for clone AA079655.

SEQ ID NO: 1691 represents the cDNA sequence for clone AA079656.

SEQ ID NO: 1692 represents the cDNA sequence for clone AA079659.

SEQ ID NO: 1693 represents the cDNA sequence for clone AA079667.

SEQ ID NO: 1694 represents the cDNA sequence for clone AA079492.

SEQ ID NO: 1695 represents the cDNA sequence for clone AA079495.

SEQ ID NO: 1696 represents the cDNA sequence for clone AA079497.

SEQ ID NO: 1697 represents the cDNA sequence for clone AA079631.

SEQ ID NO: 1698 represents the cDNA sequence for clone AA079682.

SEQ ID NO: 1699 represents the cDNA sequence for clone AA079555.

SEQ ID NO: 1700 represents the cDNA sequence for clone AA079620.

SEQ ID NO: 1701 represents the cDNA sequence for clone AA079621.

SEQ ID NO: 1702 represents the cDNA sequence for clone AA079623.

SEQ ID NO: 1703 represents the cDNA sequence for clone AA079771.

SEQ ID NO: 1704 represents the cDNA sequence for clone AA079774.

SEQ ID NO: 1705 represents the cDNA sequence for clone AA079786.

SEQ ID NO: 1706 represents the cDNA sequence for clone AA079797.

SEQ ID NO: 1707 represents the cDNA sequence for clone AA079799.

SEQ ID NO: 1708 represents the cDNA sequence for clone AA079805.

SEQ ID NO: 1709 represents the cDNA sequence for clone AA079808.

SEQ ID NO: 1710 represents the cDNA sequence for clone AA079811.

SEQ ID NO: 1711 represents the cDNA sequence for clone AA079812.

SEQ ID NO: 1712 represents the cDNA sequence for clone AA079864.

SEQ ID NO: 1713 represents the cDNA sequence for clone AA079873.

SEQ ID NO: 1714 represents the cDNA sequence for clone AA079874.

SEQ ID NO: 1715 represents the cDNA sequence for clone AA079877.

SEQ ID NO: 1716 represents the cDNA sequence for clone AA079878.

SEQ ID NO: 1717 represents the cDNA sequence for clone AA079883.

SEQ ID NO: 1718 represents the cDNA sequence for clone AA079884.

SEQ ID NO: 1719 represents the cDNA sequence for clone AA079885.

SEQ ID NO: 1720 represents the cDNA sequence for clone AA079697.

SEQ ID NO: 1721 represents the cDNA sequence for clone AA079698.

SEQ ID NO: 1722 represents the cDNA sequence for clone AA079703.

SEQ ID NO: 1723 represents the cDNA sequence for clone AA079704.

SEQ ID NO: 1724 represents the cDNA sequence for clone AA079705.

SEQ ID NO: 1725 represents the cDNA sequence for clone AA079838.

SEQ ID NO: 1726 represents the cDNA sequence for clone AA079891.

SEQ ID NO: 1727 represents the cDNA sequence for clone AA159632.

SEQ ID NO: 1728 represents the cDNA sequence for clone AA159645.

SEQ ID NO: 1729 represents the cDNA sequence for clone AA159630.

SEQ ID NO: 1730 represents the cDNA sequence for clone AA159690.

SEQ ID NO: 1731 represents the cDNA sequence for clone AA159660.

SEQ ID NO: 1732 represents the cDNA sequence for clone AA159799.

SEQ ID NO: 1733 represents the cDNA sequence for clone AA159824.

SEQ ID NO: 1734 represents the cDNA sequence for clone AA159899.

SEQ ID NO: 1735 represents the cDNA sequence for clone AA159900.

SEQ ID NO: 1736 represents the cDNA sequence for clone AA159903.

SEQ ID NO: 1737 represents the cDNA sequence for clone AA159804.

SEQ ID NO: 1738 represents the cDNA sequence for clone AA159810.

SEQ ID NO: 1739 represents the cDNA sequence for clone AA159919.

SEQ ID NO: 1740 represents the cDNA sequence for clone AA159920.

SEQ ID NO: 1741 represents the cDNA sequence for clone AA159921.

SEQ ID NO: 1742 represents the cDNA sequence for clone AA159928.

SEQ ID NO: 1743 represents the cDNA sequence for clone AA160045.

SEQ ID NO: 1744 represents the cDNA sequence for clone AA160046.

SEQ ID NO: 1745 represents the cDNA sequence for clone AA160048.

SEQ ID NO: 1746 represents the cDNA sequence for clone AA160049.

SEQ ID NO: 1747 represents the cDNA sequence for clone AA160052.

SEQ ID NO: 1748 represents the cDNA sequence for clone AA160079.

SEQ ID NO: 1749 represents the cDNA sequence for clone AA160080.

SEQ ID NO: 1750 represents the cDNA sequence for clone AA160081.

SEQ ID NO: 1751 represents the cDNA sequence for clone AA160141.

SEQ ID NO: 1752 represents the cDNA sequence for clone AA160142.

SEQ ID NO: 1753 represents the cDNA sequence for clone AA160143.

SEQ ID NO: 1754 represents the cDNA sequence for clone AA160150.

SEQ ID NO: 1755 represents the cDNA sequence for clone AA159679.

SEQ ID NO: 1756 represents the cDNA sequence for clone AA159681.

SEQ ID NO: 1757 represents the cDNA sequence for clone AA159734.

SEQ ID NO: 1758 represents the cDNA sequence for clone AA159735.

SEQ ID NO: 1759 represents the cDNA sequence for clone AA159761.

SEQ ID NO: 1760 represents the cDNA sequence for clone AA159658.

SEQ ID NO: 1761 represents the cDNA sequence for clone AA159723.

SEQ ID NO: 1762 represents the cDNA sequence for clone AA159726.

SEQ ID NO: 1763 represents the cDNA sequence for clone AA159727.

SEQ ID NO: 1764 represents the cDNA sequence for clone AA159728.

SEQ ID NO: 1765 represents the cDNA sequence for clone AA159729.

SEQ ID NO: 1766 represents the cDNA sequence for clone AA159634.

SEQ ID NO: 1767 represents the cDNA sequence for clone AA159639.

SEQ ID NO: 1768 represents the cDNA sequence for clone AA159780.

SEQ ID NO: 1769 represents the cDNA sequence for clone AA159706.

SEQ ID NO: 1770 represents the cDNA sequence for clone AA159708.

SEQ ID NO: 1771 represents the cDNA sequence for clone AA159709.

SEQ ID NO: 1772 represents the cDNA sequence for clone AA160128.

SEQ ID NO: 1773 represents the cDNA sequence for clone AA160155.

SEQ ID NO: 1774 represents the cDNA sequence for clone AA160156.

SEQ ID NO: 1775 represents the cDNA sequence for clone AA160180.

SEQ ID NO: 1776 represents the cDNA sequence for clone AA160198.

SEQ ID NO: 1777 represents the cDNA sequence for clone AA160199.

SEQ ID NO: 1778 represents the cDNA sequence for clone AA160207.

SEQ ID NO: 1779 represents the cDNA sequence for clone AA159757.

SEQ ID NO: 1780 represents the cDNA sequence for clone AA160171.

SEQ ID NO: 1781 represents the cDNA sequence for clone AA160172.

SEQ ID NO: 1782 represents the cDNA sequence for clone AA160177.

SEQ ID NO: 1783 represents the cDNA sequence for clone AA160252.

SEQ ID NO: 1784 represents the cDNA sequence for clone AA160256.

SEQ ID NO: 1785 represents the cDNA sequence for clone AA160258.

SEQ ID NO: 1786 represents the cDNA sequence for clone AA160259.

SEQ ID NO: 1787 represents the cDNA sequence for clone AA160266.

SEQ ID NO: 1788 represents the cDNA sequence for clone AA160290.

SEQ ID NO: 1789 represents the cDNA sequence for clone AA160291.

SEQ ID NO: 1790 represents the cDNA sequence for clone AA160006.

SEQ ID NO: 1791 represents the cDNA sequence for clone AA160008.

SEQ ID NO: 1792 represents the cDNA sequence for clone AA164991.

SEQ ID NO: 1793 represents the cDNA sequence for clone AA164996.

SEQ ID NO: 1794 represents the cDNA sequence for clone AA165006.

SEQ ID NO: 1795 represents the cDNA sequence for clone AA165007.

SEQ ID NO: 1796 represents the cDNA sequence for clone AA165008.

SEQ ID NO: 1797 represents the cDNA sequence for clone AA165012.

SEQ ID NO: 1798 represents the cDNA sequence for clone AA165018.

SEQ ID NO: 1799 represents the cDNA sequence for clone AA165024.

SEQ ID NO: 1800 represents the cDNA sequence for clone AA165026.

SEQ ID NO: 1801 represents the cDNA sequence for clone AA165030.

SEQ ID NO: 1802 represents the cDNA sequence for clone AA165031.

SEQ ID NO: 1803 represents the cDNA sequence for clone AA165039.

SEQ ID NO: 1804 represents the cDNA sequence for clone AA165041.

SEQ ID NO: 1805 represents the cDNA sequence for clone AA165073.

SEQ ID NO: 1806 represents the cDNA sequence for clone AA165079.

SEQ ID NO: 1807 represents the cDNA sequence for clone AA165085.

SEQ ID NO: 1808 represents the cDNA sequence for clone AA165090.

SEQ ID NO: 1809 represents the cDNA sequence for clone AA165093.

SEQ ID NO: 1810 represents the cDNA sequence for clone AA165094.

SEQ ID NO: 1811 represents the cDNA sequence for clone AA165103.

SEQ ID NO: 1812 represents the cDNA sequence for clone AA165106.

SEQ ID NO: 1813 represents the cDNA sequence for clone AA164973.

SEQ ID NO: 1814 represents the cDNA sequence for clone AA164978.

SEQ ID NO: 1815 represents the cDNA sequence for clone AA164979.

SEQ ID NO: 1816 represents the cDNA sequence for clone AA164981.

SEQ ID NO: 1817 represents the cDNA sequence for clone AA165109.

SEQ ID NO: 1818 represents the cDNA sequence for clone AA165116.

SEQ ID NO: 1819 represents the cDNA sequence for clone AA165136.

SEQ ID NO: 1820 represents the cDNA sequence for clone AA165140.

SEQ ID NO: 1821 represents the cDNA sequence for clone AA165117.

SEQ ID NO: 1822 represents the cDNA sequence for clone AA165160.

SEQ ID NO: 1823 represents the cDNA sequence for clone AA165161.

SEQ ID NO: 1824 represents the cDNA sequence for clone AA165164.

SEQ ID NO: 1825 represents the cDNA sequence for clone AA165165.

SEQ ID NO: 1826 represents the cDNA sequence for clone AA165166.

SEQ ID NO: 1827 represents the cDNA sequence for clone AA165178.

SEQ ID NO: 1828 represents the cDNA sequence for clone AA165179.

SEQ ID NO: 1829 represents the cDNA sequence for clone AA165146.

SEQ ID NO: 1830 represents the cDNA sequence for clone AA165150.

SEQ ID NO: 1831 represents the cDNA sequence for clone AA165207.

SEQ ID NO: 1832 represents the cDNA sequence for clone AA165208.

SEQ ID NO: 1833 represents the cDNA sequence for clone AA165211.

SEQ ID NO: 1834 represents the cDNA sequence for clone AA165212.

SEQ ID NO: 1835 represents the cDNA sequence for clone AA165232.

SEQ ID NO: 1836 represents the cDNA sequence for clone AA165233.

SEQ ID NO: 1837 represents the cDNA sequence for clone AA165243.

SEQ ID NO: 1838 represents the cDNA sequence for clone AA165244.

SEQ ID NO: 1839 represents the cDNA sequence for clone AA165050.

SEQ ID NO: 1840 represents the cDNA sequence for clone AA165202.

SEQ ID NO: 1841 represents the cDNA sequence for clone AA165203.

SEQ ID NO: 1842 represents the cDNA sequence for clone AA165204.

SEQ ID NO: 1843 represents the cDNA sequence for clone AA165258.

SEQ ID NO: 1844 represents the cDNA sequence for clone AA165261.

SEQ ID NO: 1845 represents the cDNA sequence for clone AA165286.

SEQ ID NO: 1846 represents the cDNA sequence for clone AA165288.

SEQ ID NO: 1847 represents the cDNA sequence for clone AA165297.

SEQ ID NO: 1848 represents the cDNA sequence for clone AA165301.

SEQ ID NO: 1849 represents the cDNA sequence for clone AA165302.

SEQ ID NO: 1850 represents the cDNA sequence for clone AA165312.

SEQ ID NO: 1851 represents the cDNA sequence for clone AA165324.

SEQ ID NO: 1852 represents the cDNA sequence for clone AA165126.

SEQ ID NO: 1853 represents the cDNA sequence for clone AA165127.

SEQ ID NO: 1854 represents the cDNA sequence for clone AA165128.

SEQ ID NO: 1855 represents the cDNA sequence for clone AA165130.

SEQ ID NO: 1856 represents the cDNA sequence for clone AA165280.

SEQ ID NO: 1857 represents the cDNA sequence for clone AA164424.

SEQ ID NO: 1858 represents the cDNA sequence for clone AA164427.

SEQ ID NO: 1859 represents the cDNA sequence for clone AA164429.

SEQ ID NO: 1860 represents the cDNA sequence for clone AA164435.

SEQ ID NO: 1861 represents the cDNA sequence for clone AA164436.

SEQ ID NO: 1862 represents the cDNA sequence for clone AA164439.

SEQ ID NO: 1863 represents the cDNA sequence for clone AA164440.

SEQ ID NO: 1864 represents the cDNA sequence for clone AA164452.

SEQ ID NO: 1865 represents the cDNA sequence for clone AA164454.

SEQ ID NO: 1866 represents the cDNA sequence for clone AA164455.

SEQ ID NO: 1867 represents the cDNA sequence for clone AA164457.

SEQ ID NO: 1868 represents the cDNA sequence for clone AA164462.

SEQ ID NO: 1869 represents the cDNA sequence for clone AA164463.

SEQ ID NO: 1870 represents the cDNA sequence for clone AA165193.

SEQ ID NO: 1871 represents the cDNA sequence for clone AA164413.

SEQ ID NO: 1872 represents the cDNA sequence for clone AA164468.

SEQ ID NO: 1873 represents the cDNA sequence for clone AA164472.

SEQ ID NO: 1874 represents the cDNA sequence for clone AA164473.

SEQ ID NO: 1875 represents the cDNA sequence for clone AA164499.

SEQ ID NO: 1876 represents the cDNA sequence for clone AA164511.

SEQ ID NO: 1877 represents the cDNA sequence for clone AA164512.

SEQ ID NO: 1878 represents the cDNA sequence for clone AA164514.

SEQ ID NO: 1879 represents the cDNA sequence for clone AA164516.

SEQ ID NO: 1880 represents the cDNA sequence for clone AA164523.

SEQ ID NO: 1881 represents the cDNA sequence for clone AA164524.

SEQ ID NO: 1882 represents the cDNA sequence for clone AA164529.

SEQ ID NO: 1883 represents the cDNA sequence for clone AA164475.

SEQ ID NO: 1884 represents the cDNA sequence for clone AA164476.

SEQ ID NO: 1885 represents the cDNA sequence for clone AA165267.

SEQ ID NO: 1886 represents the cDNA sequence for clone AA165268.

SEQ ID NO: 1887 represents the cDNA sequence for clone AA165269.

SEQ ID NO: 1888 represents the cDNA sequence for clone AA165271.

SEQ ID NO: 1889 represents the cDNA sequence for clone AA164412.

SEQ ID NO: 1890 represents the cDNA sequence for clone AA164546.

SEQ ID NO: 1891 represents the cDNA sequence for clone AA164547.

SEQ ID NO: 1892 represents the cDNA sequence for clone AA164551.

SEQ ID NO: 1893 represents the cDNA sequence for clone AA164553.

SEQ ID NO: 1894 represents the cDNA sequence for clone AA164566.

SEQ ID NO: 1895 represents the cDNA sequence for clone AA164567.

SEQ ID NO: 1896 represents the cDNA sequence for clone AA164568.

SEQ ID NO: 1897 represents the cDNA sequence for clone AA164569.

SEQ ID NO: 1898 represents the cDNA sequence for clone AA164570.

SEQ ID NO: 1899 represents the cDNA sequence for clone AA164574.

SEQ ID NO: 1900 represents the cDNA sequence for clone AA164575.

SEQ ID NO: 1901 represents the cDNA sequence for clone AA164594.

SEQ ID NO: 1902 represents the cDNA sequence for clone AA164597.

SEQ ID NO: 1903 represents the cDNA sequence for clone AA164603.

SEQ ID NO: 1904 represents the cDNA sequence for clone AA164604.

SEQ ID NO: 1905 represents the cDNA sequence for clone AA164607.

SEQ ID NO: 1906 represents the cDNA sequence for clone AA164639.

SEQ ID NO: 1907 represents the cDNA sequence for clone AA164643.

SEQ ID NO: 1908 represents the cDNA sequence for clone AA164644.

SEQ ID NO: 1909 represents the cDNA sequence for clone AA164645.

SEQ ID NO: 1910 represents the cDNA sequence for clone AA164654.

SEQ ID NO: 1911 represents the cDNA sequence for clone AA164655.

SEQ ID NO: 1912 represents the cDNA sequence for clone AA164660.

SEQ ID NO: 1913 represents the cDNA sequence for clone AA164661.

SEQ ID NO: 1914 represents the cDNA sequence for clone AA164664.

SEQ ID NO: 1915 represents the cDNA sequence for clone AA164665.

SEQ ID NO: 1916 represents the cDNA sequence for clone AA164666.

SEQ ID NO: 1917 represents the cDNA sequence for clone AA164676.

SEQ ID NO: 1918 represents the cDNA sequence for clone AA164537.

SEQ ID NO: 1919 represents the cDNA sequence for clone AA164543.

SEQ ID NO: 1920 represents the cDNA sequence for clone AA164632.

SEQ ID NO: 1921 represents the cDNA sequence for clone AA164687.

SEQ ID NO: 1922 represents the cDNA sequence for clone AA164716.

SEQ ID NO: 1923 represents the cDNA sequence for clone AA164720.

SEQ ID NO: 1924 represents the cDNA sequence for clone AA164691.

SEQ ID NO: 1925 represents the cDNA sequence for clone AA164697.

SEQ ID NO: 1926 represents the cDNA sequence for clone AA164563.

SEQ ID NO: 1927 represents the cDNA sequence for clone AA164744.

SEQ ID NO: 1928 represents the cDNA sequence for clone AA164745.

SEQ ID NO: 1929 represents the cDNA sequence for clone AA164751.

SEQ ID NO: 1930 represents the cDNA sequence for clone AA164756.

SEQ ID NO: 1931 represents the cDNA sequence for clone AA164760.

SEQ ID NO: 1932 represents the cDNA sequence for clone AA164781.

SEQ ID NO: 1933 represents the cDNA sequence for clone AA164782.

SEQ ID NO: 1934 represents the cDNA sequence for clone AA164619.

SEQ ID NO: 1935 represents the cDNA sequence for clone AA164872.

SEQ ID NO: 1936 represents the cDNA sequence for clone AA164880.

SEQ ID NO: 1937 represents the cDNA sequence for clone AA164881.

SEQ ID NO: 1938 represents the cDNA sequence for clone AA164891.

SEQ ID NO: 1939 represents the cDNA sequence for clone AA164895.

SEQ ID NO: 1940 represents the cDNA sequence for clone AA164896.

SEQ ID NO: 1941 represents the cDNA sequence for clone AA164835.

SEQ ID NO: 1942 represents the cDNA sequence for clone AA164836.

SEQ ID NO: 1943 represents the cDNA sequence for clone AA164841.

SEQ ID NO: 1944 represents the cDNA sequence for clone AA164761.

SEQ ID NO: 1945 represents the cDNA sequence for clone AA164765.

SEQ ID NO: 1946 represents the cDNA sequence for clone AA164706.

SEQ ID NO: 1947 represents the cDNA sequence for clone AA164846.

SEQ ID NO: 1948 represents the cDNA sequence for clone AA164847.

SEQ ID NO: 1949 represents the cDNA sequence for clone AA164850.

SEQ ID NO: 1950 represents the cDNA sequence for clone AA164851.

SEQ ID NO: 1951 represents the cDNA sequence for clone AA164183.

SEQ ID NO: 1952 represents the cDNA sequence for clone AA164184.

SEQ ID NO: 1953 represents the cDNA sequence for clone AA164185.

SEQ ID NO: 1954 represents the cDNA sequence for clone AA164188.

SEQ ID NO: 1955 represents the cDNA sequence for clone AA164189.

SEQ ID NO: 1956 represents the cDNA sequence for clone AA164215.

SEQ ID NO: 1957 represents the cDNA sequence for clone AA164216.

SEQ ID NO: 1958 represents the cDNA sequence for clone AA164219.

SEQ ID NO: 1959 represents the cDNA sequence for clone AA164223.

SEQ ID NO: 1960 represents the cDNA sequence for clone AA164228.

SEQ ID NO: 1961 represents the cDNA sequence for clone AA164229.

SEQ ID NO: 1962 represents the cDNA sequence for clone AA164230.

SEQ ID NO: 1963 represents the cDNA sequence for clone AA164231.

SEQ ID NO: 1964 represents the cDNA sequence for clone AA164284.

SEQ ID NO: 1965 represents the cDNA sequence for clone AA164288.

SEQ ID NO: 1966 represents the cDNA sequence for clone AA164292.

SEQ ID NO: 1967 represents the cDNA sequence for clone AA164293.

SEQ ID NO: 1968 represents the cDNA sequence for clone AA164295.

SEQ ID NO: 1969 represents the cDNA sequence for clone AA164299.

SEQ ID NO: 1970 represents the cDNA sequence for clone AA164300.

SEQ ID NO: 1971 represents the cDNA sequence for clone AA164301.

SEQ ID NO: 1972 represents the cDNA sequence for clone AA164304.

SEQ ID NO: 1973 represents the cDNA sequence for clone AA164317.

SEQ ID NO: 1974 represents the cDNA sequence for clone AA164323.

SEQ ID NO: 1975 represents the cDNA sequence for clone AA164921.

SEQ ID NO: 1976 represents the cDNA sequence for clone AA164922.

SEQ ID NO: 1977 represents the cDNA sequence for clone AA164923.

SEQ ID NO: 1978 represents the cDNA sequence for clone AA164270.

SEQ ID NO: 1979 represents the cDNA sequence for clone AA164371.

SEQ ID NO: 1980 represents the cDNA sequence for clone AA164376.

SEQ ID NO: 1981 represents the cDNA sequence for clone AA164377.

SEQ ID NO: 1982 represents the cDNA sequence for clone AA164382.

SEQ ID NO: 1983 represents the cDNA sequence for clone AA164383.

SEQ ID NO: 1984 represents the cDNA sequence for clone AA164386.

SEQ ID NO: 1985 represents the cDNA sequence for clone AA164387.

SEQ ID NO: 1986 represents the cDNA sequence for clone AA164393.

SEQ ID NO: 1987 represents the cDNA sequence for clone AA164190.

SEQ ID NO: 1988 represents the cDNA sequence for clone AA164197.

SEQ ID NO: 1989 represents the cDNA sequence for clone AA164198.

SEQ ID NO: 1990 represents the cDNA sequence for clone AA164341.

SEQ ID NO: 1991 represents the cDNA sequence for clone AA164342.

SEQ ID NO: 1992 represents the cDNA sequence for clone AA164347.

SEQ ID NO: 1993 represents the cDNA sequence for clone AA165328.

SEQ ID NO: 1994 represents the cDNA sequence for clone AA165330.

SEQ ID NO: 1995 represents the cDNA sequence for clone AA165331.

SEQ ID NO: 1996 represents the cDNA sequence for clone AA165332.

SEQ ID NO: 1997 represents the cDNA sequence for clone AA164269.

SEQ ID NO: 1998 represents the cDNA sequence for clone AA165346.

SEQ ID NO: 1999 represents the cDNA sequence for clone AA165399.

SEQ ID NO: 2000 represents the cDNA sequence for clone AA165400.

SEQ ID NO: 2001 represents the cDNA sequence for clone AA165427.

SEQ ID NO: 2002 represents the cDNA sequence for clone AA165428.

SEQ ID NO: 2003 represents the cDNA sequence for clone AA165430.

SEQ ID NO: 2004 represents the cDNA sequence for clone AA165409.

SEQ ID NO: 2005 represents the cDNA sequence for clone AA165410.

SEQ ID NO: 2006 represents the cDNA sequence for clone AA165512.

SEQ ID NO: 2007 represents the cDNA sequence for clone AA165518.

SEQ ID NO: 2008 represents the cDNA sequence for clone AA165524.

SEQ ID NO: 2009 represents the cDNA sequence for clone AA165528.

SEQ ID NO: 2010 represents the cDNA sequence for clone AA165418.

SEQ ID NO: 2011 represents the cDNA sequence for clone AA165554.

SEQ ID NO: 2012 represents the cDNA sequence for clone AA165563.

SEQ ID NO: 2013 represents the cDNA sequence for clone AA165566.

SEQ ID NO: 2014 represents the cDNA sequence for clone AA165568.

SEQ ID NO: 2015 represents the cDNA sequence for clone AA165575.

SEQ ID NO: 2016 represents the cDNA sequence for clone AA165579.

SEQ ID NO: 2017 represents the cDNA sequence for clone AA165546.

SEQ ID NO: 2018 represents the cDNA sequence for clone AA165597.

SEQ ID NO: 2019 represents the cDNA sequence for clone AA165617.

SEQ ID NO: 2020 represents the cDNA sequence for clone AA165621.

SEQ ID NO: 2021 represents the cDNA sequence for clone AA165634.

SEQ ID NO: 2022 represents the cDNA sequence for clone AA165648.

SEQ ID NO: 2023 represents the cDNA sequence for clone AA165655.

SEQ ID NO: 2024 represents the cDNA sequence for clone AA165478.

SEQ ID NO: 2025 represents the cDNA sequence for clone AA165607.

SEQ ID NO: 2026 represents the cDNA sequence for clone AA165610.

SEQ ID NO: 2027 represents the cDNA sequence for clone AA165672.

SEQ ID NO: 2028 represents the cDNA sequence for clone AA165676.

SEQ ID NO: 2029 represents the cDNA sequence for clone AA165677.

SEQ ID NO: 2030 represents the cDNA sequence for clone AA159673.

SEQ ID NO: 2031 represents the cDNA sequence for clone AA159675.

SEQ ID NO: 2032 represents the cDNA sequence for clone AA159696.

SEQ ID NO: 2033 represents the cDNA sequence for clone AA159701.

SEQ ID NO: 2034 represents the cDNA sequence for clone AA159702.

SEQ ID NO: 2035 represents the cDNA sequence for clone AA159688.

SEQ ID NO: 2036 represents the cDNA sequence for clone AA159689.

SEQ ID NO: 2037 represents the cDNA sequence for clone AA159740.

SEQ ID NO: 2038 represents the cDNA sequence for clone AA159743.

SEQ ID NO: 2039 represents the cDNA sequence for clone AA159744.

SEQ ID NO: 2040 represents the cDNA sequence for clone AA159746.

SEQ ID NO: 2041 represents the cDNA sequence for clone AA159769.

SEQ ID NO: 2042 represents the cDNA sequence for clone AA159772.

SEQ ID NO: 2043 represents the cDNA sequence for clone AA159775.

SEQ ID NO: 2044 represents the cDNA sequence for clone AA159600.

SEQ ID NO: 2045 represents the cDNA sequence for clone AA159605.

SEQ ID NO: 2046 represents the cDNA sequence for clone AA159613.

SEQ ID NO: 2047 represents the cDNA sequence for clone AA159614.

SEQ ID NO: 2048 represents the cDNA sequence for clone AA159665.

SEQ ID NO: 2049 represents the cDNA sequence for clone AA159667.

SEQ ID NO: 2050 represents the cDNA sequence for clone AA166695.

SEQ ID NO: 2051 represents the cDNA sequence for clone AA166715.

SEQ ID NO: 2052 represents the cDNA sequence for clone AA166721.

SEQ ID NO: 2053 represents the cDNA sequence for clone AA166727.

SEQ ID NO: 2054 represents the cDNA sequence for clone AA166729.

SEQ ID NO: 2055 represents the cDNA sequence for clone AA166734.

SEQ ID NO: 2056 represents the cDNA sequence for clone AA166739.

SEQ ID NO: 2057 represents the cDNA sequence for clone AA166697.

SEQ ID NO: 2058 represents the cDNA sequence for clone AA166699.

SEQ ID NO: 2059 represents the cDNA sequence for clone AA166700.

SEQ ID NO: 2060 represents the cDNA sequence for clone AA166701.

SEQ ID NO: 2061 represents the cDNA sequence for clone AA166753.

SEQ ID NO: 2062 represents the cDNA sequence for clone AA166756.

SEQ ID NO: 2063 represents the cDNA sequence for clone AA166620.

SEQ ID NO: 2064 represents the cDNA sequence for clone AA166743.

SEQ ID NO: 2065 represents the cDNA sequence for clone AA166746.

SEQ ID NO: 2066 represents the cDNA sequence for clone AA166757.

SEQ ID NO: 2067 represents the cDNA sequence for clone AA166763.

SEQ ID NO: 2068 represents the cDNA sequence for clone AA166765.

SEQ ID NO: 2069 represents the cDNA sequence for clone AA166785.

SEQ ID NO: 2070 represents the cDNA sequence for clone AA166786.

SEQ ID NO: 2071 represents the cDNA sequence for clone AA166791.

SEQ ID NO: 2072 represents the cDNA sequence for clone AA166797.

SEQ ID NO: 2073 represents the cDNA sequence for clone AA166798.

SEQ ID NO: 2074 represents the cDNA sequence for clone AA166799.

SEQ ID NO: 2075 represents the cDNA sequence for clone AA166800.

SEQ ID NO: 2076 represents the cDNA sequence for clone AA166803.

SEQ ID NO: 2077 represents the cDNA sequence for clone AA166805.

SEQ ID NO: 2078 represents the cDNA sequence for clone AA166808.

SEQ ID NO: 2079 represents the cDNA sequence for clone AA166817.

SEQ ID NO: 2080 represents the cDNA sequence for clone AA166819.

SEQ ID NO: 2081 represents the cDNA sequence for clone AA166821.

SEQ ID NO: 2082 represents the cDNA sequence for clone AA166639.

SEQ ID NO: 2083 represents the cDNA sequence for clone AA166898.

SEQ ID NO: 2084 represents the cDNA sequence for clone AA166934.

SEQ ID NO: 2085 represents the cDNA sequence for clone AA166937.

SEQ ID NO: 2086 represents the cDNA sequence for clone AA166938.

SEQ ID NO: 2087 represents the cDNA sequence for clone AA166944.

SEQ ID NO: 2088 represents the cDNA sequence for clone AA166946.

SEQ ID NO: 2089 represents the cDNA sequence for clone AA166948.

SEQ ID NO: 2090 represents the cDNA sequence for clone AA166951.

SEQ ID NO: 2091 represents the cDNA sequence for clone AA166952.

SEQ ID NO: 2092 represents the cDNA sequence for clone AA166963.

SEQ ID NO: 2093 represents the cDNA sequence for clone AA166965.

SEQ ID NO: 2094 represents the cDNA sequence for clone AA166966.

SEQ ID NO: 2095 represents the cDNA sequence for clone AA166709.

SEQ ID NO: 2096 represents the cDNA sequence for clone AA166710.

SEQ ID NO: 2097 represents the cDNA sequence for clone AA166919.

SEQ ID NO: 2098 represents the cDNA sequence for clone AA166970.

SEQ ID NO: 2099 represents the cDNA sequence for clone AA166971.

SEQ ID NO: 2100 represents the cDNA sequence for clone AA166972.

SEQ ID NO: 2101 represents the cDNA sequence for clone AA166974.

SEQ ID NO: 2102 represents the cDNA sequence for clone AA167006.

SEQ ID NO: 2103 represents the cDNA sequence for clone AA167008.

SEQ ID NO: 2104 represents the cDNA sequence for clone AA167020.

SEQ ID NO: 2105 represents the cDNA sequence for clone AA167023.

SEQ ID NO: 2106 represents the cDNA sequence for clone AA167029.

SEQ ID NO: 2107 represents the cDNA sequence for clone AA166911.

SEQ ID NO: 2108 represents the cDNA sequence for clone AA167041.

SEQ ID NO: 2109 represents the cDNA sequence for clone AA167044.

SEQ ID NO: 2110 represents the cDNA sequence for clone AA167067.

SEQ ID NO: 2111 represents the cDNA sequence for clone AA167068.

SEQ ID NO: 2112 represents the cDNA sequence for clone AA167078.

SEQ ID NO: 2113 represents the cDNA sequence for clone AA167080.

SEQ ID NO: 2114 represents the cDNA sequence for clone AA167082.

SEQ ID NO: 2115 represents the cDNA sequence for clone AA167084.

SEQ ID NO: 2116 represents the cDNA sequence for clone AA167088.

SEQ ID NO: 2117 represents the cDNA sequence for clone AA167098.

SEQ ID NO: 2118 represents the cDNA sequence for clone AA167099.

SEQ ID NO: 2119 represents the cDNA sequence for clone AA167102.

SEQ ID NO: 2120 represents the cDNA sequence for clone AA167103.

SEQ ID NO: 2121 represents the cDNA sequence for clone AA167104.

SEQ ID NO: 2122 represents the cDNA sequence for clone AA166980.

SEQ ID NO: 2123 represents the cDNA sequence for clone AA166982.

SEQ ID NO: 2124 represents the cDNA sequence for clone AA166986.

SEQ ID NO: 2125 represents the cDNA sequence for clone AA167062.

SEQ ID NO: 2126 represents the cDNA sequence for clone AA167065.

SEQ ID NO: 2127 represents the cDNA sequence for clone AA167114.

SEQ ID NO: 2128 represents the cDNA sequence for clone AA167144.

SEQ ID NO: 2129 represents the cDNA sequence for clone AA167145.

SEQ ID NO: 2130 represents the cDNA sequence for clone AA167149.

SEQ ID NO: 2131 represents the cDNA sequence for clone AA167150.

SEQ ID NO: 2132 represents the cDNA sequence for clone AA167155.

SEQ ID NO: 2133 represents the cDNA sequence for clone AA167156.

SEQ ID NO: 2134 represents the cDNA sequence for clone AA167157.

SEQ ID NO: 2135 represents the cDNA sequence for clone AA167162.

SEQ ID NO: 2136 represents the cDNA sequence for clone AA167163.

SEQ ID NO: 2137 represents the cDNA sequence for clone AA167316.

SEQ ID NO: 2138 represents the cDNA sequence for clone AA167320.

SEQ ID NO: 2139 represents the cDNA sequence for clone AA167191.

SEQ ID NO: 2140 represents the cDNA sequence for clone AA167195.

SEQ ID NO: 2141 represents the cDNA sequence for clone AA167269.

SEQ ID NO: 2142 represents the cDNA sequence for clone AA167270.

SEQ ID NO: 2143 represents the cDNA sequence for clone AA167323.

SEQ ID NO: 2144 represents the cDNA sequence for clone AA167324.

SEQ ID NO: 2145 represents the cDNA sequence for clone AA167329.

SEQ ID NO: 2146 represents the cDNA sequence for clone AA167333.

SEQ ID NO: 2147 represents the cDNA sequence for clone AA167338.

SEQ ID NO: 2148 represents the cDNA sequence for clone AA167370.

SEQ ID NO: 2149 represents the cDNA sequence for clone AA167374.

SEQ ID NO: 2150 represents the cDNA sequence for clone AA167377.

SEQ ID NO: 2151 represents the cDNA sequence for clone AA167378.

SEQ ID NO: 2152 represents the cDNA sequence for clone AA167381.

SEQ ID NO: 2153 represents the cDNA sequence for clone AA167382.

SEQ ID NO: 2154 represents the cDNA sequence for clone AA167199.

SEQ ID NO: 2155 represents the cDNA sequence for clone AA167204.

SEQ ID NO: 2156 represents the cDNA sequence for clone AA167205.

SEQ ID NO: 2157 represents the cDNA sequence for clone AA167394.

SEQ ID NO: 2158 represents the cDNA sequence for clone AA167406.

SEQ ID NO: 2159 represents the cDNA sequence for clone AA167431.

SEQ ID NO: 2160 represents the cDNA sequence for clone AA167432.

SEQ ID NO: 2161 represents the cDNA sequence for clone AA167437.

SEQ ID NO: 2162 represents the cDNA sequence for clone AA169469.

SEQ ID NO: 2163 represents the cDNA sequence for clone AA169470.

SEQ ID NO: 2164 represents the cDNA sequence for clone AA169474.

SEQ ID NO: 2165 represents the cDNA sequence for clone AA169475.

SEQ ID NO: 2166 represents the cDNA sequence for clone AA169329.

SEQ ID NO: 2167 represents the cDNA sequence for clone AA169336.

SEQ ID NO: 2168 represents the cDNA sequence for clone AA169498.

SEQ ID NO: 2169 represents the cDNA sequence for clone AA169503.

SEQ ID NO: 2170 represents the cDNA sequence for clone AA169504.

SEQ ID NO: 2171 represents the cDNA sequence for clone AA169513.

SEQ ID NO: 2172 represents the cDNA sequence for clone AA169516.

SEQ ID NO: 2173 represents the cDNA sequence for clone AA169543.

SEQ ID NO: 2174 represents the cDNA sequence for clone AA169531.

SEQ ID NO: 2175 represents the cDNA sequence for clone AA169560.

SEQ ID NO: 2176 represents the cDNA sequence for clone AA169573.

SEQ ID NO: 2177 represents the cDNA sequence for clone AA169578.

SEQ ID NO: 2178 represents the cDNA sequence for clone AA169583.

SEQ ID NO: 2179 represents the cDNA sequence for clone AA169535.

SEQ ID NO: 2180 represents the cDNA sequence for clone AA169594.

SEQ ID NO: 2181 represents the cDNA sequence for clone AA169599.

SEQ ID NO: 2182 represents the cDNA sequence for clone AA169602.

SEQ ID NO: 2183 represents the cDNA sequence for clone AA169606.

SEQ ID NO: 2184 represents the cDNA sequence for clone AA169609.

SEQ ID NO: 2185 represents the cDNA sequence for clone AA169612.

SEQ ID NO: 2186 represents the cDNA sequence for clone AA169623.

SEQ ID NO: 2187 represents the cDNA sequence for clone AA169625.

SEQ ID NO: 2188 represents the cDNA sequence for clone AA169650.

SEQ ID NO: 2189 represents the cDNA sequence for clone AA169651.

SEQ ID NO: 2190 represents the cDNA sequence for clone AA169654.

SEQ ID NO: 2191 represents the cDNA sequence for clone AA169656.

SEQ ID NO: 2192 represents the cDNA sequence for clone AA169660.

SEQ ID NO: 2193 represents the cDNA sequence for clone AA169662.

SEQ ID NO: 2194 represents the cDNA sequence for clone AA169663.

SEQ ID NO: 2195 represents the cDNA sequence for clone AA169668.

SEQ ID NO: 2196 represents the cDNA sequence for clone AA169670.

SEQ ID NO: 2197 represents the cDNA sequence for clone AA169676.

SEQ ID NO: 2198 represents the cDNA sequence for clone AA169678.

SEQ ID NO: 2199 represents the cDNA sequence for clone AA169679.

SEQ ID NO: 2200 represents the cDNA sequence for clone AA169680.

SEQ ID NO: 2201 represents the cDNA sequence for clone AA169682.

SEQ ID NO: 2202 represents the cDNA sequence for clone AA169706.

SEQ ID NO: 2203 represents the cDNA sequence for clone AA169707.

SEQ ID NO: 2204 represents the cDNA sequence for clone AA169830.

SEQ ID NO: 2205 represents the cDNA sequence for clone AA169831.

SEQ ID NO: 2206 represents the cDNA sequence for clone AA169834.

SEQ ID NO: 2207 represents the cDNA sequence for clone AA169204.

SEQ ID NO: 2208 represents the cDNA sequence for clone AA169488.

SEQ ID NO: 2209 represents the cDNA sequence for clone AA169493.

SEQ ID NO: 2210 represents the cDNA sequence for clone AA169588.

SEQ ID NO: 2211 represents the cDNA sequence for clone AA169640.

SEQ ID NO: 2212 represents the cDNA sequence for clone AA169641.

SEQ ID NO: 2213 represents the cDNA sequence for clone AA169642.

SEQ ID NO: 2214 represents the cDNA sequence for clone AA169644.

SEQ ID NO: 2215 represents the cDNA sequence for clone AA169645.

SEQ ID NO: 2216 represents the cDNA sequence for clone AA169807.

SEQ ID NO: 2217 represents the cDNA sequence for clone AA169808.

SEQ ID NO: 2218 represents the cDNA sequence for clone AA169810.

SEQ ID NO: 2219 represents the cDNA sequence for clone AA169811.

SEQ ID NO: 2220 represents the cDNA sequence for clone AA169120.

SEQ ID NO: 2221 represents the cDNA sequence for clone AA169121.

SEQ ID NO: 2222 represents the cDNA sequence for clone AA169122.

SEQ ID NO: 2223 represents the cDNA sequence for clone AA169124.

SEQ ID NO: 2224 represents the cDNA sequence for clone AA169300.

SEQ ID NO: 2225 represents the cDNA sequence for clone AA169307.

SEQ ID NO: 2226 represents the cDNA sequence for clone AA169630.

SEQ ID NO: 2227 represents the cDNA sequence for clone AA169633.

SEQ ID NO: 2228 represents the cDNA sequence for clone AA169635.

SEQ ID NO: 2229 represents the cDNA sequence for clone AA169377.

SEQ ID NO: 2230 represents the cDNA sequence for clone AA169379.

SEQ ID NO: 2231 represents the cDNA sequence for clone AA169394.

SEQ ID NO: 2232 represents the cDNA sequence for clone AA169396.

SEQ ID NO: 2233 represents the cDNA sequence for clone AA169398.

SEQ ID NO: 2234 represents the cDNA sequence for clone AA169400.

SEQ ID NO: 2235 represents the cDNA sequence for clone AA169405.

SEQ ID NO: 2236 represents the cDNA sequence for clone AA169411.

SEQ ID NO: 2237 represents the cDNA sequence for clone AA169412.

SEQ ID NO: 2238 represents the cDNA sequence for clone AA169733.

SEQ ID NO: 2239 represents the cDNA sequence for clone AA169361.

SEQ ID NO: 2240 represents the cDNA sequence for clone AA169366.

SEQ ID NO: 2241 represents the cDNA sequence for clone AA169415.

SEQ ID NO: 2242 represents the cDNA sequence for clone AA169419.

SEQ ID NO: 2243 represents the cDNA sequence for clone AA169424.

SEQ ID NO: 2244 represents the cDNA sequence for clone AA169430.

SEQ ID NO: 2245 represents the cDNA sequence for clone AA169435.

SEQ ID NO: 2246 represents the cDNA sequence for clone AA169437.

SEQ ID NO: 2247 represents the cDNA sequence for clone AA169444.

SEQ ID NO: 2248 represents the cDNA sequence for clone AA169352.

SEQ ID NO: 2249 represents the cDNA sequence for clone AA169355.

SEQ ID NO: 2250 represents the cDNA sequence for clone AA169838.

SEQ ID NO: 2251 represents the cDNA sequence for clone AA169840.

SEQ ID NO: 2252 represents the cDNA sequence for clone AA169882.

SEQ ID NO: 2253 represents the cDNA sequence for clone AA169883.

SEQ ID NO: 2254 represents the cDNA sequence for clone AA169886.

SEQ ID NO: 2255 represents the cDNA sequence for clone AA171635.

SEQ ID NO: 2256 represents the cDNA sequence for clone AA171636.

SEQ ID NO: 2257 represents the cDNA sequence for clone AA171637.

SEQ ID NO: 2258 represents the cDNA sequence for clone AA171639.

SEQ ID NO: 2259 represents the cDNA sequence for clone AA171640.

SEQ ID NO: 2260 represents the cDNA sequence for clone AA171645.

SEQ ID NO: 2261 represents the cDNA sequence for clone AA171646.

SEQ ID NO: 2262 represents the cDNA sequence for clone AA171647.

SEQ ID NO: 2263 represents the cDNA sequence for clone AA171648.

SEQ ID NO: 2264 represents the cDNA sequence for clone AA171654.

SEQ ID NO: 2265 represents the cDNA sequence for clone AA171657.

SEQ ID NO: 2266 represents the cDNA sequence for clone AA171669.

SEQ ID NO: 2267 represents the cDNA sequence for clone AA171675.

SEQ ID NO: 2268 represents the cDNA sequence for clone AA171677.

SEQ ID NO: 2269 represents the cDNA sequence for clone AA171426.

SEQ ID NO: 2270 represents the cDNA sequence for clone AA171688.

SEQ ID NO: 2271 represents the cDNA sequence for clone AA171692.

SEQ ID NO: 2272 represents the cDNA sequence for clone AA171702.

SEQ ID NO: 2273 represents the cDNA sequence for clone AA171750.

SEQ ID NO: 2274 represents the cDNA sequence for clone AA171609.

SEQ ID NO: 2275 represents the cDNA sequence for clone AA171755.

SEQ ID NO: 2276 represents the cDNA sequence for clone AA171760.

SEQ ID NO: 2277 represents the cDNA sequence for clone AA171793.

SEQ ID NO: 2278 represents the cDNA sequence for clone AA171797.

SEQ ID NO: 2279 represents the cDNA sequence for clone AA171803.

SEQ ID NO: 2280 represents the cDNA sequence for clone AA171806.

SEQ ID NO: 2281 represents the cDNA sequence for clone AA171807.

SEQ ID NO: 2282 represents the cDNA sequence for clone AA171814.

SEQ ID NO: 2283 represents the cDNA sequence for clone AA171818.

SEQ ID NO: 2284 represents the cDNA sequence for clone AA171626.

SEQ ID NO: 2285 represents the cDNA sequence for clone AA171631.

SEQ ID NO: 2286 represents the cDNA sequence for clone AA171951.

SEQ ID NO: 2287 represents the cDNA sequence for clone AA171960.

SEQ ID NO: 2288 represents the cDNA sequence for clone AA171762.

SEQ ID NO: 2289 represents the cDNA sequence for clone AA171766.

SEQ ID NO: 2290 represents the cDNA sequence for clone AA171768.

SEQ ID NO: 2291 represents the cDNA sequence for clone AA171913.

SEQ ID NO: 2292 represents the cDNA sequence for clone AA171968.

SEQ ID NO: 2293 represents the cDNA sequence for clone AA171974.

SEQ ID NO: 2294 represents the cDNA sequence for clone AA171994.

SEQ ID NO: 2295 represents the cDNA sequence for clone AA171996.

SEQ ID NO: 2296 represents the cDNA sequence for clone AA172000.

SEQ ID NO: 2297 represents the cDNA sequence for clone AA172001.

SEQ ID NO: 2298 represents the cDNA sequence for clone AA172008.

SEQ ID NO: 2299 represents the cDNA sequence for clone AA172020.

SEQ ID NO: 2300 represents the cDNA sequence for clone AA172039.

SEQ ID NO: 2301 represents the cDNA sequence for clone AA171844.

SEQ ID NO: 2302 represents the cDNA sequence for clone AA171984.

SEQ ID NO: 2303 represents the cDNA sequence for clone AA171985.

SEQ ID NO: 2304 represents the cDNA sequence for clone AA172046.

SEQ ID NO: 2305 represents the cDNA sequence for clone AA172047.

SEQ ID NO: 2306 represents the cDNA sequence for clone AA172051.

SEQ ID NO: 2307 represents the cDNA sequence for clone AA172052.

SEQ ID NO: 2308 represents the cDNA sequence for clone AA172053.

SEQ ID NO: 2309 represents the cDNA sequence for clone AA172056.

SEQ ID NO: 2310 represents the cDNA sequence for clone AA172097.

SEQ ID NO: 2311 represents the cDNA sequence for clone AA172099.

SEQ ID NO: 2312 represents the cDNA sequence for clone AA172199.

SEQ ID NO: 2313 represents the cDNA sequence for clone AA172224.

SEQ ID NO: 2314 represents the cDNA sequence for clone AA172225.

SEQ ID NO: 2315 represents the cDNA sequence for clone AA172227.

SEQ ID NO: 2316 represents the cDNA sequence for clone AA172236.

SEQ ID NO: 2317 represents the cDNA sequence for clone AA172238.

SEQ ID NO: 2318 represents the cDNA sequence for clone AA172243.

SEQ ID NO: 2319 represents the cDNA sequence for clone AA172247.

SEQ ID NO: 2320 represents the cDNA sequence for clone AA172248.

SEQ ID NO: 2321 represents the cDNA sequence for clone AA172064.

SEQ ID NO: 2322 represents the cDNA sequence for clone AA172219.

SEQ ID NO: 2323 represents the cDNA sequence for clone AA172291.

SEQ ID NO: 2324 represents the cDNA sequence for clone AA172293.

SEQ ID NO: 2325 represents the cDNA sequence for clone AA172297.

SEQ ID NO: 2326 represents the cDNA sequence for clone AA172298.

SEQ ID NO: 2327 represents the cDNA sequence for clone AA172300.

SEQ ID NO: 2328 represents the cDNA sequence for clone AA172301.

SEQ ID NO: 2329 represents the cDNA sequence for clone AA172303.

SEQ ID NO: 2330 represents the cDNA sequence for clone AA172306.

SEQ ID NO: 2331 represents the cDNA sequence for clone AA172370.

SEQ ID NO: 2332 represents the cDNA sequence for clone AA172376.

SEQ ID NO: 2333 represents the cDNA sequence for clone AA172385.

SEQ ID NO: 2334 represents the cDNA sequence for clone AA172387.

SEQ ID NO: 2335 represents the cDNA sequence for clone AA172213.

SEQ ID NO: 2336 represents the cDNA sequence for clone AA173381.

SEQ ID NO: 2337 represents the cDNA sequence for clone AA173403.

SEQ ID NO: 2338 represents the cDNA sequence for clone AA173409.

SEQ ID NO: 2339 represents the cDNA sequence for clone AA173417.

SEQ ID NO: 2340 represents the cDNA sequence for clone AA173420.

SEQ ID NO: 2341 represents the cDNA sequence for clone AA173428.

SEQ ID NO: 2342 represents the cDNA sequence for clone AA173431.

SEQ ID NO: 2343 represents the cDNA sequence for clone AA173433.

SEQ ID NO: 2344 represents the cDNA sequence for clone AA173436.

SEQ ID NO: 2345 represents the cDNA sequence for clone AA173440.

SEQ ID NO: 2346 represents the cDNA sequence for clone AA173444.

SEQ ID NO: 2347 represents the cDNA sequence for clone AA173447.

SEQ ID NO: 2348 represents the cDNA sequence for clone AA173463.

SEQ ID NO: 2349 represents the cDNA sequence for clone AA173464.

SEQ ID NO: 2350 represents the cDNA sequence for clone AA173465.

SEQ ID NO: 2351 represents the cDNA sequence for clone AA173466.

SEQ ID NO: 2352 represents the cDNA sequence for clone AA173507.

SEQ ID NO: 2353 represents the cDNA sequence for clone AA173509.

SEQ ID NO: 2354 represents the cDNA sequence for clone AA173510.

SEQ ID NO: 2355 represents the cDNA sequence for clone AA173511.

SEQ ID NO: 2356 represents the cDNA sequence for clone AA173512.

SEQ ID NO: 2357 represents the cDNA sequence for clone AA173515.

SEQ ID NO: 2358 represents the cDNA sequence for clone AA173518.

SEQ ID NO: 2359 represents the cDNA sequence for clone AA173325.

SEQ ID NO: 2360 represents the cDNA sequence for clone AA173327.

SEQ ID NO: 2361 represents the cDNA sequence for clone AA173493.

SEQ ID NO: 2362 represents the cDNA sequence for clone AA173494.

SEQ ID NO: 2363 represents the cDNA sequence for clone AA173495.

SEQ ID NO: 2364 represents the cDNA sequence for clone AA173499.

SEQ ID NO: 2365 represents the cDNA sequence for clone AA173500.

SEQ ID NO: 2366 represents the cDNA sequence for clone AA173501.

SEQ ID NO: 2367 represents the cDNA sequence for clone AA173520.

SEQ ID NO: 2368 represents the cDNA sequence for clone AA173521.

SEQ ID NO: 2369 represents the cDNA sequence for clone AA173523.

SEQ ID NO: 2370 represents the cDNA sequence for clone AA173525.

SEQ ID NO: 2371 represents the cDNA sequence for clone AA173564.

SEQ ID NO: 2372 represents the cDNA sequence for clone AA173577.

SEQ ID NO: 2373 represents the cDNA sequence for clone AA173583.

SEQ ID NO: 2374 represents the cDNA sequence for clone AA173588.

SEQ ID NO: 2375 represents the cDNA sequence for clone AA173383.

SEQ ID NO: 2376 represents the cDNA sequence for clone AA173384.

SEQ ID NO: 2377 represents the cDNA sequence for clone AA173389.

SEQ ID NO: 2378 represents the cDNA sequence for clone AA173538.

SEQ ID NO: 2379 represents the cDNA sequence for clone AA173596.

SEQ ID NO: 2380 represents the cDNA sequence for clone AA173618.

SEQ ID NO: 2381 represents the cDNA sequence for clone AA173622.

SEQ ID NO: 2382 represents the cDNA sequence for clone AA173628.

SEQ ID NO: 2383 represents the cDNA sequence for clone AA173631.

SEQ ID NO: 2384 represents the cDNA sequence for clone AA173632.

SEQ ID NO: 2385 represents the cDNA sequence for clone AA173635.

SEQ ID NO: 2386 represents the cDNA sequence for clone AA173638.

SEQ ID NO: 2387 represents the cDNA sequence for clone AA173639.

SEQ ID NO: 2388 represents the cDNA sequence for clone AA173640.

SEQ ID NO: 2389 represents the cDNA sequence for clone AA173645.

SEQ ID NO: 2390 represents the cDNA sequence for clone AA173646.

SEQ ID NO: 2391 represents the cDNA sequence for clone AA173659.

SEQ ID NO: 2392 represents the cDNA sequence for clone AA173610.

SEQ ID NO: 2393 represents the cDNA sequence for clone AA173665.

SEQ ID NO: 2394 represents the cDNA sequence for clone AA173666.

SEQ ID NO: 2395 represents the cDNA sequence for clone AA173671.

SEQ ID NO: 2396 represents the cDNA sequence for clone AA173709.

SEQ ID NO: 2397 represents the cDNA sequence for clone AA173713.

SEQ ID NO: 2398 represents the cDNA sequence for clone AA173716.

SEQ ID NO: 2399 represents the cDNA sequence for clone AA173721.

SEQ ID NO: 2400 represents the cDNA sequence for clone AA173727.

SEQ ID NO: 2401 represents the cDNA sequence for clone AA173729.

SEQ ID NO: 2402 represents the cDNA sequence for clone AA173530.

SEQ ID NO: 2403 represents the cDNA sequence for clone AA173535.

SEQ ID NO: 2404 represents the cDNA sequence for clone AA173536.

SEQ ID NO: 2405 represents the cDNA sequence for clone AA173699.

SEQ ID NO: 2406 represents the cDNA sequence for clone AA173702.

SEQ ID NO: 2407 represents the cDNA sequence for clone AA173735.

SEQ ID NO: 2408 represents the cDNA sequence for clone AA173736.

SEQ ID NO: 2409 represents the cDNA sequence for clone AA173739.

SEQ ID NO: 2410 represents the cDNA sequence for clone AA173740.

SEQ ID NO: 2411 represents the cDNA sequence for clone AA173868.

SEQ ID NO: 2412 represents the cDNA sequence for clone AA173746.

SEQ ID NO: 2413 represents the cDNA sequence for clone AA173823.

SEQ ID NO: 2414 represents the cDNA sequence for clone AA173826.

SEQ ID NO: 2415 represents the cDNA sequence for clone AA173830.

SEQ ID NO: 2416 represents the cDNA sequence for clone AA173872.

SEQ ID NO: 2417 represents the cDNA sequence for clone AA173878.

SEQ ID NO: 2418 represents the cDNA sequence for clone AA173879.

SEQ ID NO: 2419 represents the cDNA sequence for clone AA173897.

SEQ ID NO: 2420 represents the cDNA sequence for clone AA173901.

SEQ ID NO: 2421 represents the cDNA sequence for clone AA173907.

SEQ ID NO: 2422 represents the cDNA sequence for clone AA173923.

SEQ ID NO: 2423 represents the cDNA sequence for clone AA173926.

SEQ ID NO: 2424 represents the cDNA sequence for clone AA173928.

SEQ ID NO: 2425 represents the cDNA sequence for clone AA173938.

SEQ ID NO: 2426 represents the cDNA sequence for clone AA173939.

SEQ ID NO: 2427 represents the cDNA sequence for clone AA173759.

SEQ ID NO: 2428 represents the cDNA sequence for clone AA173888.

SEQ ID NO: 2429 represents the cDNA sequence for clone AA173942.

SEQ ID NO: 2430 represents the cDNA sequence for clone AA173970.

SEQ ID NO: 2431 represents the cDNA sequence for clone AA173978.

SEQ ID NO: 2432 represents the cDNA sequence for clone AA173983.

SEQ ID NO: 2433 represents the cDNA sequence for clone AA173988.

SEQ ID NO: 2434 represents the cDNA sequence for clone AA173994.

SEQ ID NO: 2435 represents the cDNA sequence for clone AA173955.

SEQ ID NO: 2436 represents the cDNA sequence for clone AA173956.

SEQ ID NO: 2437 represents the cDNA sequence for clone AA174006.

SEQ ID NO: 2438 represents the cDNA sequence for clone AA174008.

SEQ ID NO: 2439 represents the cDNA sequence for clone AA173813.

SEQ ID NO: 2440 represents the cDNA sequence for clone AA173817.

SEQ ID NO: 2441 represents the cDNA sequence for clone AA173881.

SEQ ID NO: 2442 represents the cDNA sequence for clone AA173884.

SEQ ID NO: 2443 represents the cDNA sequence for clone AA173886.

SEQ ID NO: 2444 represents the cDNA sequence for clone AA174022.

SEQ ID NO: 2445 represents the cDNA sequence for clone AA174023.

SEQ ID NO: 2446 represents the cDNA sequence for clone AA174030.

SEQ ID NO: 2447 represents the cDNA sequence for clone AA174044.

SEQ ID NO: 2448 represents the cDNA sequence for clone AA174046.

SEQ ID NO: 2449 represents the cDNA sequence for clone AA174145.

SEQ ID NO: 2450 represents the cDNA sequence for clone AA174004.

SEQ ID NO: 2451 represents the cDNA sequence for clone AA174094.

SEQ ID NO: 2452 represents the cDNA sequence for clone AA173029.

SEQ ID NO: 2453 represents the cDNA sequence for clone AA173033.

SEQ ID NO: 2454 represents the cDNA sequence for clone AA173046.

SEQ ID NO: 2455 represents the cDNA sequence for clone AA173055.

SEQ ID NO: 2456 represents the cDNA sequence for clone AA173058.

SEQ ID NO: 2457 represents the cDNA sequence for clone AA173172.

SEQ ID NO: 2458 represents the cDNA sequence for clone AA173188.

SEQ ID NO: 2459 represents the cDNA sequence for clone AA173196.

SEQ ID NO: 2460 represents the cDNA sequence for clone AA173166.

SEQ ID NO: 2461 represents the cDNA sequence for clone AA173199.

SEQ ID NO: 2462 represents the cDNA sequence for clone AA176202.

SEQ ID NO: 2463 represents the cDNA sequence for clone AA176225.

SEQ ID NO: 2464 represents the cDNA sequence for clone AA176226.

SEQ ID NO: 2465 represents the cDNA sequence for clone AA176249.

SEQ ID NO: 2466 represents the cDNA sequence for clone AA176253.

SEQ ID NO: 2467 represents the cDNA sequence for clone AA176257.

SEQ ID NO: 2468 represents the cDNA sequence for clone AA176090.

SEQ ID NO: 2469 represents the cDNA sequence for clone AA176304.

SEQ ID NO: 2470 represents the cDNA sequence for clone AA176305.

SEQ ID NO: 2471 represents the cDNA sequence for clone AA176312.

SEQ ID NO: 2472 represents the cDNA sequence for clone AA176313.

SEQ ID NO: 2473 represents the cDNA sequence for clone AA176315.

SEQ ID NO: 2474 represents the cDNA sequence for clone AA176273.

SEQ ID NO: 2475 represents the cDNA sequence for clone AA176323.

SEQ ID NO: 2476 represents the cDNA sequence for clone AA176324.

SEQ ID NO: 2477 represents the cDNA sequence for clone AA176328.

SEQ ID NO: 2478 represents the cDNA sequence for clone AA176330.

SEQ ID NO: 2479 represents the cDNA sequence for clone AA176690.

SEQ ID NO: 2480 represents the cDNA sequence for clone AA176693.

SEQ ID NO: 2481 represents the cDNA sequence for clone AA176702.

SEQ ID NO: 2482 represents the cDNA sequence for clone AA176710.

SEQ ID NO: 2483 represents the cDNA sequence for clone AA176713.

SEQ ID NO: 2484 represents the cDNA sequence for clone AA176717.

SEQ ID NO: 2485 represents the cDNA sequence for clone AA176528.

SEQ ID NO: 2486 represents the cDNA sequence for clone AA180008.

SEQ ID NO: 2487 represents the cDNA sequence for clone AA180011.

SEQ ID NO: 2488 represents the cDNA sequence for clone AA180012.

SEQ ID NO: 2489 represents the cDNA sequence for clone AA180031.

SEQ ID NO: 2490 represents the cDNA sequence for clone AA180032.

SEQ ID NO: 2491 represents the cDNA sequence for clone AA180033.

SEQ ID NO: 2492 represents the cDNA sequence for clone AA180037.

SEQ ID NO: 2493 represents the cDNA sequence for clone AA180039.

SEQ ID NO: 2494 represents the cDNA sequence for clone AA235450.

SEQ ID NO: 2495 represents the cDNA sequence for clone AA235315.

SEQ ID NO: 2496 represents the cDNA sequence for clone AA235316.

SEQ ID NO: 2497 represents the cDNA sequence for clone AA235401.

SEQ ID NO: 2498 represents the cDNA sequence for clone AA235403.

SEQ ID NO: 2499 represents the cDNA sequence for clone AA235454.

SEQ ID NO: 2500 represents the cDNA sequence for clone AA235455.

SEQ ID NO: 2501 represents the cDNA sequence for clone AA235492.

SEQ ID NO: 2502 represents the cDNA sequence for clone AA235504.

SEQ ID NO: 2503 represents the cDNA sequence for clone AA235512.

SEQ ID NO: 2504 represents the cDNA sequence for clone AA235329.

SEQ ID NO: 2505 represents the cDNA sequence for clone AA235523.

SEQ ID NO: 2506 represents the cDNA sequence for clone AA235551.

SEQ ID NO: 2507 represents the cDNA sequence for clone AA235562.

SEQ ID NO: 2508 represents the cDNA sequence for clone AA235534.

SEQ ID NO: 2509 represents the cDNA sequence for clone AA235536.

SEQ ID NO: 2510 represents the cDNA sequence for clone AA235538.

SEQ ID NO: 2511 represents the cDNA sequence for clone AA235576.

SEQ ID NO: 2512 represents the cDNA sequence for clone AA235387.

SEQ ID NO: 2513 represents the cDNA sequence for clone AA235392.

SEQ ID NO: 2514 represents the cDNA sequence for clone AA235393.

SEQ ID NO: 2515 represents the cDNA sequence for clone AA235572.

SEQ ID NO: 2516 represents the cDNA sequence for clone AA235544.

SEQ ID NO: 2517 represents the cDNA sequence for clone AA235546.

SEQ ID NO: 2518 represents the cDNA sequence for clone AA235547.

SEQ ID NO: 2519 represents the cDNA sequence for clone AA235604.

SEQ ID NO: 2520 represents the cDNA sequence for clone AA235626.

SEQ ID NO: 2521 represents the cDNA sequence for clone AA235633.

SEQ ID NO: 2522 represents the cDNA sequence for clone AA235636.

SEQ ID NO: 2523 represents the cDNA sequence for clone AA235641.

SEQ ID NO: 2524 represents the cDNA sequence for clone AA235654.

SEQ ID NO: 2525 represents the cDNA sequence for clone AA235660.

SEQ ID NO: 2526 represents the cDNA sequence for clone AA235662.

SEQ ID NO: 2527 represents the cDNA sequence for clone AA235663.

SEQ ID NO: 2528 represents the cDNA sequence for clone AA235466.

SEQ ID NO: 2529 represents the cDNA sequence for clone AA235664.

SEQ ID NO: 2530 represents the cDNA sequence for clone AA235668.

SEQ ID NO: 2531 represents the cDNA sequence for clone AA235693.

SEQ ID NO: 2532 represents the cDNA sequence for clone AA235698.

SEQ ID NO: 2533 represents the cDNA sequence for clone AA235701.

SEQ ID NO: 2534 represents the cDNA sequence for clone AA235704.

SEQ ID NO: 2535 represents the cDNA sequence for clone AA235709.

SEQ ID NO: 2536 represents the cDNA sequence for clone AA235710.

SEQ ID NO: 2537 represents the cDNA sequence for clone AA235712.

SEQ ID NO: 2538 represents the cDNA sequence for clone AA235721.

SEQ ID NO: 2539 represents the cDNA sequence for clone AA235723.

SEQ ID NO: 2540 represents the cDNA sequence for clone AA235687.

SEQ ID NO: 2541 represents the cDNA sequence for clone AA235742.

SEQ ID NO: 2542 represents the cDNA sequence for clone AA236698.

SEQ ID NO: 2543 represents the cDNA sequence for clone AA236706.

SEQ ID NO: 2544 represents the cDNA sequence for clone AA236711.

SEQ ID NO: 2545 represents the cDNA sequence for clone AA236712.

SEQ ID NO: 2546 represents the cDNA sequence for clone AA236719.

SEQ ID NO: 2547 represents the cDNA sequence for clone AA236730.

SEQ ID NO: 2548 represents the cDNA sequence for clone AA236671.

SEQ ID NO: 2549 represents the cDNA sequence for clone AA236672.

SEQ ID NO: 2550 represents the cDNA sequence for clone AA250734.

SEQ ID NO: 2551 represents the cDNA sequence for clone AA250735.

SEQ ID NO: 2552 represents the cDNA sequence for clone AA250736.

SEQ ID NO: 2553 represents the cDNA sequence for clone AA250737.

SEQ ID NO: 2554 represents the cDNA sequence for clone AA250739.

SEQ ID NO: 2555 represents the cDNA sequence for clone AA250767.

SEQ ID NO: 2556 represents the cDNA sequence for clone AA250771.

SEQ ID NO: 2557 represents the cDNA sequence for clone AA250776.

SEQ ID NO: 2558 represents the cDNA sequence for clone AA250782.

SEQ ID NO: 2559 represents the cDNA sequence for clone AA250762.

SEQ ID NO: 2560 represents the cDNA sequence for clone AA250763.

SEQ ID NO: 2561 represents the cDNA sequence for clone AA250765.

SEQ ID NO: 2562 represents the cDNA sequence for clone AA282991.

SEQ ID NO: 2563 represents the cDNA sequence for clone AA283183.

SEQ ID NO: 2564 represents the cDNA sequence for clone AA283185.

SEQ ID NO: 2565 represents the cDNA sequence for clone AA283198.

SEQ ID NO: 2566 represents the cDNA sequence for clone AA283199.

SEQ ID NO: 2567 represents the cDNA sequence for clone AA283192.

SEQ ID NO: 2568 represents the cDNA sequence for clone AA283204.

SEQ ID NO: 2569 represents the cDNA sequence for clone AA284701.

SEQ ID NO: 2570 represents the cDNA sequence for clone AA284703.

SEQ ID NO: 2571 represents the cDNA sequence for clone AA284730.

SEQ ID NO: 2572 represents the cDNA sequence for clone AA284731.

SEQ ID NO: 2573 represents the cDNA sequence for clone AA284733.

SEQ ID NO: 2574 represents the cDNA sequence for clone AA284736.

SEQ ID NO: 2575 represents the cDNA sequence for clone AA284740.

SEQ ID NO: 2576 represents the cDNA sequence for clone AA284741.

SEQ ID NO: 2577 represents the cDNA sequence for clone AA284742.

SEQ ID NO: 2578 represents the cDNA sequence for clone AA284743.

SEQ ID NO: 2579 represents the cDNA sequence for clone AA284747.

SEQ ID NO: 2580 represents the cDNA sequence for clone AA284752.

SEQ ID NO: 2581 represents the cDNA sequence for clone AA284754.

SEQ ID NO: 2582 represents the cDNA sequence for clone AA284772.

SEQ ID NO: 2583 represents the cDNA sequence for clone AA284683.

SEQ ID NO: 2584 represents the cDNA sequence for clone AA284684.

SEQ ID NO: 2585 represents the cDNA sequence for clone AA284685.

SEQ ID NO: 2586 represents the cDNA sequence for clone AA284721.

SEQ ID NO: 2587 represents the cDNA sequence for clone AA284726.

SEQ ID NO: 2588 represents the cDNA sequence for clone AA284775.

SEQ ID NO: 2589 represents the cDNA sequence for clone AA284776.

SEQ ID NO: 2590 represents the cDNA sequence for clone AA284777.

SEQ ID NO: 2591 represents the cDNA sequence for clone AA284782.

SEQ ID NO: 2592 represents the cDNA sequence for clone AA284811.

SEQ ID NO: 2593 represents the cDNA sequence for clone AA284815.

SEQ ID NO: 2594 represents the cDNA sequence for clone AA284818.

SEQ ID NO: 2595 represents the cDNA sequence for clone AA284823.

SEQ ID NO: 2596 represents the cDNA sequence for clone AA284790.

SEQ ID NO: 2597 represents the cDNA sequence for clone AA284841.

SEQ ID NO: 2598 represents the cDNA sequence for clone AA284692.

SEQ ID NO: 2599 represents the cDNA sequence for clone AA284693.

SEQ ID NO: 2600 represents the cDNA sequence for clone AA284826.

SEQ ID NO: 2601 represents the cDNA sequence for clone AA284830.

SEQ ID NO: 2602 represents the cDNA sequence for clone AA284832.

SEQ ID NO: 2603 represents the cDNA sequence for clone AA284842.

SEQ ID NO: 2604 represents the cDNA sequence for clone AA284845.

SEQ ID NO: 2605 represents the cDNA sequence for clone AA284847.

SEQ ID NO: 2606 represents the cDNA sequence for clone AA284850.

SEQ ID NO: 2607 represents the cDNA sequence for clone AA284855.

SEQ ID NO: 2608 represents the cDNA sequence for clone AA284858.

SEQ ID NO: 2609 represents the cDNA sequence for clone AA284881.

SEQ ID NO: 2610 represents the cDNA sequence for clone AA284882.

SEQ ID NO: 2611 represents the cDNA sequence for clone AA284891.

SEQ ID NO: 2612 represents the cDNA sequence for clone AA284898.

SEQ ID NO: 2613 represents the cDNA sequence for clone AA284902.

SEQ ID NO: 2614 represents the cDNA sequence for clone AA284903.

SEQ ID NO: 2615 represents the cDNA sequence for clone AA284910.

SEQ ID NO: 2616 represents the cDNA sequence for clone AA284711.

SEQ ID NO: 2617 represents the cDNA sequence for clone AA284718.

SEQ ID NO: 2618 represents the cDNA sequence for clone AA284871.

SEQ ID NO: 2619 represents the cDNA sequence for clone AA284874.

SEQ ID NO: 2620 represents the cDNA sequence for clone AA284542.

SEQ ID NO: 2621 represents the cDNA sequence for clone AA284545.

SEQ ID NO: 2622 represents the cDNA sequence for clone AA284552.

SEQ ID NO: 2623 represents the cDNA sequence for clone AA284580.

SEQ ID NO: 2624 represents the cDNA sequence for clone AA284581.

SEQ ID NO: 2625 represents the cDNA sequence for clone AA284585.

SEQ ID NO: 2626 represents the cDNA sequence for clone AA284587.

SEQ ID NO: 2627 represents the cDNA sequence for clone AA284588.

SEQ ID NO: 2628 represents the cDNA sequence for clone AA284589.

SEQ ID NO: 2629 represents the cDNA sequence for clone AA284591.

SEQ ID NO: 2630 represents the cDNA sequence for clone AA284598.

SEQ ID NO: 2631 represents the cDNA sequence for clone AA284602.

SEQ ID NO: 2632 represents the cDNA sequence for clone AA284606.

SEQ ID NO: 2633 represents the cDNA sequence for clone AA284798.

SEQ ID NO: 2634 represents the cDNA sequence for clone AA284800.

SEQ ID NO: 2635 represents the cDNA sequence for clone AA284570.

SEQ ID NO: 2636 represents the cDNA sequence for clone AA284574.

SEQ ID NO: 2637 represents the cDNA sequence for clone AA284611.

SEQ ID NO: 2638 represents the cDNA sequence for clone AA284612.

SEQ ID NO: 2639 represents the cDNA sequence for clone AA284613.

SEQ ID NO: 2640 represents the cDNA sequence for clone AA284637.

SEQ ID NO: 2641 represents the cDNA sequence for clone AA284639.

SEQ ID NO: 2642 represents the cDNA sequence for clone AA284643.

SEQ ID NO: 2643 represents the cDNA sequence for clone AA284651.

SEQ ID NO: 2644 represents the cDNA sequence for clone AA284653.

SEQ ID NO: 2645 represents the cDNA sequence for clone AA284654.

SEQ ID NO: 2646 represents the cDNA sequence for clone AA284658.

SEQ ID NO: 2647 represents the cDNA sequence for clone AA284659.

SEQ ID NO: 2648 represents the cDNA sequence for clone AA284666.

SEQ ID NO: 2649 represents the cDNA sequence for clone AA284675.

SEQ ID NO: 2650 represents the cDNA sequence for clone AA284676.

SEQ ID NO: 2651 represents the cDNA sequence for clone AA284678.

SEQ ID NO: 2652 represents the cDNA sequence for clone AA284558.

SEQ ID NO: 2653 represents the cDNA sequence for clone AA284564.

SEQ ID NO: 2654 represents the cDNA sequence for clone AA284565.

SEQ ID NO: 2655 represents the cDNA sequence for clone AA284915.

SEQ ID NO: 2656 represents the cDNA sequence for clone AA284966.

SEQ ID NO: 2657 represents the cDNA sequence for clone AA284968.

SEQ ID NO: 2658 represents the cDNA sequence for clone AA284970.

SEQ ID NO: 2659 represents the cDNA sequence for clone AA284978.

SEQ ID NO: 2660 represents the cDNA sequence for clone AA284619.

SEQ ID NO: 2661 represents the cDNA sequence for clone AA284623.

SEQ ID NO: 2662 represents the cDNA sequence for clone AA284986.

SEQ ID NO: 2663 represents the cDNA sequence for clone AA284989.

SEQ ID NO: 2664 represents the cDNA sequence for clone AA284990.

SEQ ID NO: 2665 represents the cDNA sequence for clone AA285009.

SEQ ID NO: 2666 represents the cDNA sequence for clone AA285015.

SEQ ID NO: 2667 represents the cDNA sequence for clone AA285021.

SEQ ID NO: 2668 represents the cDNA sequence for clone AA285030.

SEQ ID NO: 2669 represents the cDNA sequence for clone AA283613.

SEQ ID NO: 2670 represents the cDNA sequence for clone AA283614.

SEQ ID NO: 2671 represents the cDNA sequence for clone AA285061.

SEQ ID NO: 2672 represents the cDNA sequence for clone AA285090.

SEQ ID NO: 2673 represents the cDNA sequence for clone AA285093.

SEQ ID NO: 2674 represents the cDNA sequence for clone AA285097.

SEQ ID NO: 2675 represents the cDNA sequence for clone AA285098.

SEQ ID NO: 2676 represents the cDNA sequence for clone AA285105.

SEQ ID NO: 2677 represents the cDNA sequence for clone AA285119.

SEQ ID NO: 2678 represents the cDNA sequence for clone AA285124.

SEQ ID NO: 2679 represents the cDNA sequence for clone AA284991.

SEQ ID NO: 2680 represents the cDNA sequence for clone AA284993.

SEQ ID NO: 2681 represents the cDNA sequence for clone AA284996.

SEQ ID NO: 2682 represents the cDNA sequence for clone AA284997.

SEQ ID NO: 2683 represents the cDNA sequence for clone AA283621.

SEQ ID NO: 2684 represents the cDNA sequence for clone AA283622.

SEQ ID NO: 2685 represents the cDNA sequence for clone AA283674.

SEQ ID NO: 2686 represents the cDNA sequence for clone AA283687.

SEQ ID NO: 2687 represents the cDNA sequence for clone AA283737.

SEQ ID NO: 2688 represents the cDNA sequence for clone AA283739.

SEQ ID NO: 2689 represents the cDNA sequence for clone AA283740.

SEQ ID NO: 2690 represents the cDNA sequence for clone AA283763.

SEQ ID NO: 2691 represents the cDNA sequence for clone AA283766.

SEQ ID NO: 2692 represents the cDNA sequence for clone AA283772.

SEQ ID NO: 2693 represents the cDNA sequence for clone AA283784.

SEQ ID NO: 2694 represents the cDNA sequence for clone AA283791.

SEQ ID NO: 2695 represents the cDNA sequence for clone AA283794.

SEQ ID NO: 2696 represents the cDNA sequence for clone AA283796.

SEQ ID NO: 2697 represents the cDNA sequence for clone AA283747.

SEQ ID NO: 2698 represents the cDNA sequence for clone AA283753.

SEQ ID NO: 2699 represents the cDNA sequence for clone AA283853.

SEQ ID NO: 2700 represents the cDNA sequence for clone AA283854.

SEQ ID NO: 2701 represents the cDNA sequence for clone AA283855.

SEQ ID NO: 2702 represents the cDNA sequence for clone AA283857.

SEQ ID NO: 2703 represents the cDNA sequence for clone AA284322.

SEQ ID NO: 2704 represents the cDNA sequence for clone AA284499.

SEQ ID NO: 2705 represents the cDNA sequence for clone AA284515.

SEQ ID NO: 2706 represents the cDNA sequence for clone AA284530.

SEQ ID NO: 2707 represents the cDNA sequence for clone AA284537.

SEQ ID NO: 2708 represents the cDNA sequence for clone AA284508.

SEQ ID NO: 2709 represents the cDNA sequence for clone AA287132.

SEQ ID NO: 2710 represents the cDNA sequence for clone AA287150.

SEQ ID NO: 2711 represents the cDNA sequence for clone AA287151.

SEQ ID NO: 2712 represents the cDNA sequence for clone AA287153.

SEQ ID NO: 2713 represents the cDNA sequence for clone AA287211.

SEQ ID NO: 2714 represents the cDNA sequence for clone AA287217.

SEQ ID NO: 2715 represents the cDNA sequence for clone AA287229.

SEQ ID NO: 2716 represents the cDNA sequence for clone AA287230.

SEQ ID NO: 2717 represents the cDNA sequence for clone AA287237.

SEQ ID NO: 2718 represents the cDNA sequence for clone AA287238.

SEQ ID NO: 2719 represents the cDNA sequence for clone AA287239.

SEQ ID NO: 2720 represents the cDNA sequence for clone AA287073.

SEQ ID NO: 2721 represents the cDNA sequence for clone AA287223.

SEQ ID NO: 2722 represents the cDNA sequence for clone AA287248.

SEQ ID NO: 2723 represents the cDNA sequence for clone AA287251.

SEQ ID NO: 2724 represents the cDNA sequence for clone AA290739.

SEQ ID NO: 2725 represents the cDNA sequence for clone AA290740.

SEQ ID NO: 2726 represents the cDNA sequence for clone AA290743.

SEQ ID NO: 2727 represents the cDNA sequence for clone AA290744.

SEQ ID NO: 2728 represents the cDNA sequence for clone AA290745.

SEQ ID NO: 2729 represents the cDNA sequence for clone AA290748.

SEQ ID NO: 2730 represents the cDNA sequence for clone AA290749.

SEQ ID NO: 2731 represents the cDNA sequence for clone AA290753.

SEQ ID NO: 2732 represents the cDNA sequence for clone AA290754.

SEQ ID NO: 2733 represents the cDNA sequence for clone AA290765.

SEQ ID NO: 2734 represents the cDNA sequence for clone AA290767.

SEQ ID NO: 2735 represents the cDNA sequence for clone AA290773.

SEQ ID NO: 2736 represents the cDNA sequence for clone AA290774.

SEQ ID NO: 2737 represents the cDNA sequence for clone AA290776.

SEQ ID NO: 2738 represents the cDNA sequence for clone AA290786.

SEQ ID NO: 2739 represents the cDNA sequence for clone AA290787.

SEQ ID NO: 2740 represents the cDNA sequence for clone AA290791.

SEQ ID NO: 2741 represents the cDNA sequence for clone AA290811.

SEQ ID NO: 2742 represents the cDNA sequence for clone AA290813.

SEQ ID NO: 2743 represents the cDNA sequence for clone AA290827.

SEQ ID NO: 2744 represents the cDNA sequence for clone AA290830.

SEQ ID NO: 2745 represents the cDNA sequence for clone AA290831.

SEQ ID NO: 2746 represents the cDNA sequence for clone AA290832.

SEQ ID NO: 2747 represents the cDNA sequence for clone AA290835.

SEQ ID NO: 2748 represents the cDNA sequence for clone AA290837.

SEQ ID NO: 2749 represents the cDNA sequence for clone AA290838.

SEQ ID NO: 2750 represents the cDNA sequence for clone AA290840.

SEQ ID NO: 2751 represents the cDNA sequence for clone AA290794.

SEQ ID NO: 2752 represents the cDNA sequence for clone AA290709.

SEQ ID NO: 2753 represents the cDNA sequence for clone AA290712.

SEQ ID NO: 2754 represents the cDNA sequence for clone AA290844.

SEQ ID NO: 2755 represents the cDNA sequence for clone AA290848.

SEQ ID NO: 2756 represents the cDNA sequence for clone AA290849.

SEQ ID NO: 2757 represents the cDNA sequence for clone AA290851.

SEQ ID NO: 2758 represents the cDNA sequence for clone AA290853.

SEQ ID NO: 2759 represents the cDNA sequence for clone AA290856.

SEQ ID NO: 2760 represents the cDNA sequence for clone AA290857.

SEQ ID NO: 2761 represents the cDNA sequence for clone AA290858.

SEQ ID NO: 2762 represents the cDNA sequence for clone AA290895.

SEQ ID NO: 2763 represents the cDNA sequence for clone AA290901.

SEQ ID NO: 2764 represents the cDNA sequence for clone AA290903.

SEQ ID NO: 2765 represents the cDNA sequence for clone AA290961.

SEQ ID NO: 2766 represents the cDNA sequence for clone AA290962.

SEQ ID NO: 2767 represents the cDNA sequence for clone AA290985.

SEQ ID NO: 2768 represents the cDNA sequence for clone AA290986.

SEQ ID NO: 2769 represents the cDNA sequence for clone AA290987.

SEQ ID NO: 2770 represents the cDNA sequence for clone AA290800.

SEQ ID NO: 2771 represents the cDNA sequence for clone AA290801.

SEQ ID NO: 2772 represents the cDNA sequence for clone AA290805.

SEQ ID NO: 2773 represents the cDNA sequence for clone AA290860.

SEQ ID NO: 2774 represents the cDNA sequence for clone AA290863.

SEQ ID NO: 2775 represents the cDNA sequence for clone AA290865.

SEQ ID NO: 2776 represents the cDNA sequence for clone AA290867.

SEQ ID NO: 2777 represents the cDNA sequence for clone AA290868.

SEQ ID NO: 2778 represents the cDNA sequence for clone AA290581.

SEQ ID NO: 2779 represents the cDNA sequence for clone AA290582.

SEQ ID NO: 2780 represents the cDNA sequence for clone AA290585.

SEQ ID NO: 2781 represents the cDNA sequence for clone AA290589.

SEQ ID NO: 2782 represents the cDNA sequence for clone AA290643.

SEQ ID NO: 2783 represents the cDNA sequence for clone AA290666.

SEQ ID NO: 2784 represents the cDNA sequence for clone AA290684.

SEQ ID NO: 2785 represents the cDNA sequence for clone AA291043.

SEQ ID NO: 2786 represents the cDNA sequence for clone AA291046.

SEQ ID NO: 2787 represents the cDNA sequence for clone AA291047.

SEQ ID NO: 2788 represents the cDNA sequence for clone AA291048.

SEQ ID NO: 2789 represents the cDNA sequence for clone AA291049.

SEQ ID NO: 2790 represents the cDNA sequence for clone AA291051.

SEQ ID NO: 2791 represents the cDNA sequence for clone AA291052.

SEQ ID NO: 2792 represents the cDNA sequence for clone AA290647.

SEQ ID NO: 2793 represents the cDNA sequence for clone AA290650.

SEQ ID NO: 2794 represents the cDNA sequence for clone AA291022.

SEQ ID NO: 2795 represents the cDNA sequence for clone AA291027.

SEQ ID NO: 2796 represents the cDNA sequence for clone AA291056.

SEQ ID NO: 2797 represents the cDNA sequence for clone AA291059.

SEQ ID NO: 2798 represents the cDNA sequence for clone AA291364.

SEQ ID NO: 2799 represents the cDNA sequence for clone AA291371.

SEQ ID NO: 2800 represents the cDNA sequence for clone AA291374.

SEQ ID NO: 2801 represents the cDNA sequence for clone AA291375.

SEQ ID NO: 2802 represents the cDNA sequence for clone AA291377.

SEQ ID NO: 2803 represents the cDNA sequence for clone AA291379.

SEQ ID NO: 2804 represents the cDNA sequence for clone AA291383.

SEQ ID NO: 2805 represents the cDNA sequence for clone AA291395.

SEQ ID NO: 2806 represents the cDNA sequence for clone AA291398.

SEQ ID NO: 2807 represents the cDNA sequence for clone AA291404.

SEQ ID NO: 2808 represents the cDNA sequence for clone AA291202.

SEQ ID NO: 2809 represents the cDNA sequence for clone AA291204.

SEQ ID NO: 2810 represents the cDNA sequence for clone AA291207.

SEQ ID NO: 2811 represents the cDNA sequence for clone AA291210.

SEQ ID NO: 2812 represents the cDNA sequence for clone AA291355.

SEQ ID NO: 2813 represents the cDNA sequence for clone AA291356.

SEQ ID NO: 2814 represents the cDNA sequence for clone AA291357.

SEQ ID NO: 2815 represents the cDNA sequence for clone AA291360.

SEQ ID NO: 2816 represents the cDNA sequence for clone AA291409.

SEQ ID NO: 2817 represents the cDNA sequence for clone AA291411.

SEQ ID NO: 2818 represents the cDNA sequence for clone AA291438.

SEQ ID NO: 2819 represents the cDNA sequence for clone AA291439.

SEQ ID NO: 2820 represents the cDNA sequence for clone AA291441.

SEQ ID NO: 2821 represents the cDNA sequence for clone AA291442.

SEQ ID NO: 2822 represents the cDNA sequence for clone AA291443.

SEQ ID NO: 2823 represents the cDNA sequence for clone AA291455.

SEQ ID NO: 2824 represents the cDNA sequence for clone AA291457.

SEQ ID NO: 2825 represents the cDNA sequence for clone AA291458.

SEQ ID NO: 2826 represents the cDNA sequence for clone AA291459.

SEQ ID NO: 2827 represents the cDNA sequence for clone AA291472.

SEQ ID NO: 2828 represents the cDNA sequence for clone AA291286.

SEQ ID NO: 2829 represents the cDNA sequence for clone AA291430.

SEQ ID NO: 2830 represents the cDNA sequence for clone AA291475.

SEQ ID NO: 2831 represents the cDNA sequence for clone AA291503.

SEQ ID NO: 2832 represents the cDNA sequence for clone AA291512.

SEQ ID NO: 2833 represents the cDNA sequence for clone AA291514.

SEQ ID NO: 2834 represents the cDNA sequence for clone AA291515.

SEQ ID NO: 2835 represents the cDNA sequence for clone AA291522.

SEQ ID NO: 2836 represents the cDNA sequence for clone AA291523.

SEQ ID NO: 2837 represents the cDNA sequence for clone AA291485.

SEQ ID NO: 2838 represents the cDNA sequence for clone AA291488.

SEQ ID NO: 2839 represents the cDNA sequence for clone AA291536.

SEQ ID NO: 2840 represents the cDNA sequence for clone AA291494.

SEQ ID NO: 2841 represents the cDNA sequence for clone AA291495.

SEQ ID NO: 2842 represents the cDNA sequence for clone AA291499.

SEQ ID NO: 2843 represents the cDNA sequence for clone AA291548.

SEQ ID NO: 2844 represents the cDNA sequence for clone AA291584.

SEQ ID NO: 2845 represents the cDNA sequence for clone AA291588.

SEQ ID NO: 2846 represents the cDNA sequence for clone AA291590.

SEQ ID NO: 2847 represents the cDNA sequence for clone AA291600.

SEQ ID NO: 2848 represents the cDNA sequence for clone AA291606.

SEQ ID NO: 2849 represents the cDNA sequence for clone AA291608.

SEQ ID NO: 2850 represents the cDNA sequence for clone AA291611.

SEQ ID NO: 2851 represents the cDNA sequence for clone AA291348.

SEQ ID NO: 2852 represents the cDNA sequence for clone AA291351.

SEQ ID NO: 2853 represents the cDNA sequence for clone AA291353.

SEQ ID NO: 2854 represents the cDNA sequence for clone AA291562.

SEQ ID NO: 2855 represents the cDNA sequence for clone AA291565.

SEQ ID NO: 2856 represents the cDNA sequence for clone AA291615.

SEQ ID NO: 2857 represents the cDNA sequence for clone AA291617.

SEQ ID NO: 2858 represents the cDNA sequence for clone AA291639.

SEQ ID NO: 2859 represents the cDNA sequence for clone AA291644.

SEQ ID NO: 2860 represents the cDNA sequence for clone AA291646.

SEQ ID NO: 2861 represents the cDNA sequence for clone AA291660.

SEQ ID NO: 2862 represents the cDNA sequence for clone AA291672.

SEQ ID NO: 2863 represents the cDNA sequence for clone AA291530.

SEQ ID NO: 2864 represents the cDNA sequence for clone AA291631.

SEQ ID NO: 2865 represents the cDNA sequence for clone AA291633.

SEQ ID NO: 2866 represents the cDNA sequence for clone AA291634.

SEQ ID NO: 2867 represents the cDNA sequence for clone AA291635.

SEQ ID NO: 2868 represents the cDNA sequence for clone AA291638.

SEQ ID NO: 2869 represents the cDNA sequence for clone AA291685.

SEQ ID NO: 2870 represents the cDNA sequence for clone AA291687.

SEQ ID NO: 2871 represents the cDNA sequence for clone AA291706.

SEQ ID NO: 2872 represents the cDNA sequence for clone AA291713.

SEQ ID NO: 2873 represents the cDNA sequence for clone AA291716.

SEQ ID NO: 2874 represents the cDNA sequence for clone AA291717.

SEQ ID NO: 2875 represents the cDNA sequence for clone AA291724.

SEQ ID NO: 2876 represents the cDNA sequence for clone AA291739.

SEQ ID NO: 2877 represents the cDNA sequence for clone AA291741.

SEQ ID NO: 2878 represents the cDNA sequence for clone AA291742.

SEQ ID NO: 2879 represents the cDNA sequence for clone AA291553.

SEQ ID NO: 2880 represents the cDNA sequence for clone AA291625.

SEQ ID NO: 2881 represents the cDNA sequence for clone AA291750.

SEQ ID NO: 2882 represents the cDNA sequence for clone AA291751.

SEQ ID NO: 2883 represents the cDNA sequence for clone AA291754.

SEQ ID NO: 2884 represents the cDNA sequence for clone AA291756.

SEQ ID NO: 2885 represents the cDNA sequence for clone AA291762.

SEQ ID NO: 2886 represents the cDNA sequence for clone AA291793.

SEQ ID NO: 2887 represents the cDNA sequence for clone AA291800.

SEQ ID NO: 2888 represents the cDNA sequence for clone AA291803.

SEQ ID NO: 2889 represents the cDNA sequence for clone AA291806.

SEQ ID NO: 2890 represents the cDNA sequence for clone AA291692.

SEQ ID NO: 2891 represents the cDNA sequence for clone AA291695.

SEQ ID NO: 2892 represents the cDNA sequence for clone AA291779.

SEQ ID NO: 2893 represents the cDNA sequence for clone AA291783.

SEQ ID NO: 2894 represents the cDNA sequence for clone AA291817.

SEQ ID NO: 2895 represents the cDNA sequence for clone AA291818.

SEQ ID NO: 2896 represents the cDNA sequence for clone AA291820.

SEQ ID NO: 2897 represents the cDNA sequence for clone AA291860.

SEQ ID NO: 2898 represents the cDNA sequence for clone AA291862.

SEQ ID NO: 2899 represents the cDNA sequence for clone AA291864.

SEQ ID NO: 2900 represents the cDNA sequence for clone AA291865.

SEQ ID NO: 2901 represents the cDNA sequence for clone AA291871.

SEQ ID NO: 2902 represents the cDNA sequence for clone AA291877.

SEQ ID NO: 2903 represents the cDNA sequence for clone AA291884.

SEQ ID NO: 2904 represents the cDNA sequence for clone AA291697.

SEQ ID NO: 2905 represents the cDNA sequence for clone AA291704.

SEQ ID NO: 2906 represents the cDNA sequence for clone AA291705.

SEQ ID NO: 2907 represents the cDNA sequence for clone AA291835.

SEQ ID NO: 2908 represents the cDNA sequence for clone AA291837.

SEQ ID NO: 2909 represents the cDNA sequence for clone AA291838.

SEQ ID NO: 2910 represents the cDNA sequence for clone AA291887.

SEQ ID NO: 2911 represents the cDNA sequence for clone AA291892.

SEQ ID NO: 2912 represents the cDNA sequence for clone AA291912.

SEQ ID NO: 2913 represents the cDNA sequence for clone AA291914.

SEQ ID NO: 2914 represents the cDNA sequence for clone AA291898.

SEQ ID NO: 2915 represents the cDNA sequence for clone AA291902.

SEQ ID NO: 2916 represents the cDNA sequence for clone AA291904.

SEQ ID NO: 2917 represents the cDNA sequence for clone AA291905.

SEQ ID NO: 2918 represents the cDNA sequence for clone AA291765.

SEQ ID NO: 2919 represents the cDNA sequence for clone AA291930.

SEQ ID NO: 2920 represents the cDNA sequence for clone AA291931.

SEQ ID NO: 2921 represents the cDNA sequence for clone AA291964.

SEQ ID NO: 2922 represents the cDNA sequence for clone AA291983.

SEQ ID NO: 2923 represents the cDNA sequence for clone AA291989.

SEQ ID NO: 2924 represents the cDNA sequence for clone AA291994.

SEQ ID NO: 2925 represents the cDNA sequence for clone AA291996.

SEQ ID NO: 2926 represents the cDNA sequence for clone AA291997.

SEQ ID NO: 2927 represents the cDNA sequence for clone AA292013.

SEQ ID NO: 2928 represents the cDNA sequence for clone AA292015.

SEQ ID NO: 2929 represents the cDNA sequence for clone AA292018.

SEQ ID NO: 2930 represents the cDNA sequence for clone AA292021.

SEQ ID NO: 2931 represents the cDNA sequence for clone AA292023.

SEQ ID NO: 2932 represents the cDNA sequence for clone AA291827.

SEQ ID NO: 2933 represents the cDNA sequence for clone AA291828.

SEQ ID NO: 2934 represents the cDNA sequence for clone AA291974.

SEQ ID NO: 2935 represents the cDNA sequence for clone AA291981.

SEQ ID NO: 2936 represents the cDNA sequence for clone AA292072.

SEQ ID NO: 2937 represents the cDNA sequence for clone AA292075.

SEQ ID NO: 2938 represents the cDNA sequence for clone AA292077.

SEQ ID NO: 2939 represents the cDNA sequence for clone AA292084.

SEQ ID NO: 2940 represents the cDNA sequence for clone AA292085.

SEQ ID NO: 2941 represents the cDNA sequence for clone AA292087.

SEQ ID NO: 2942 represents the cDNA sequence for clone AA292148.

SEQ ID NO: 2943 represents the cDNA sequence for clone AA292157.

SEQ ID NO: 2944 represents the cDNA sequence for clone AA292166.

SEQ ID NO: 2945 represents the cDNA sequence for clone AA292026.

SEQ ID NO: 2946 represents the cDNA sequence for clone AA292029.

SEQ ID NO: 2947 represents the cDNA sequence for clone AA292034.

SEQ ID NO: 2948 represents the cDNA sequence for clone AA292121.

SEQ ID NO: 2949 represents the cDNA sequence for clone AA292122.

SEQ ID NO: 2950 represents the cDNA sequence for clone AA292123.

SEQ ID NO: 2951 represents the cDNA sequence for clone AA292113.

SEQ ID NO: 2952 represents the cDNA sequence for clone AA292180.

SEQ ID NO: 2953 represents the cDNA sequence for clone AA292182.

SEQ ID NO: 2954 represents the cDNA sequence for clone AA292186.

SEQ ID NO: 2955 represents the cDNA sequence for clone AA292195.

SEQ ID NO: 2956 represents the cDNA sequence for clone AA292196.

SEQ ID NO: 2957 represents the cDNA sequence for clone AA292201.

SEQ ID NO: 2958 represents the cDNA sequence for clone AA292204.

SEQ ID NO: 2959 represents the cDNA sequence for clone AA292206.

SEQ ID NO: 2960 represents the cDNA sequence for clone AA292208.

SEQ ID NO: 2961 represents the cDNA sequence for clone AA292214.

SEQ ID NO: 2962 represents the cDNA sequence for clone AA292169.

SEQ ID NO: 2963 represents the cDNA sequence for clone AA292174.

SEQ ID NO: 2964 represents the cDNA sequence for clone AA292234.

SEQ ID NO: 2965 represents the cDNA sequence for clone AA292044.

SEQ ID NO: 2966 represents the cDNA sequence for clone AA292052.

SEQ ID NO: 2967 represents the cDNA sequence for clone AA292245.

SEQ ID NO: 2968 represents the cDNA sequence for clone AA292266.

SEQ ID NO: 2969 represents the cDNA sequence for clone AA292275.

SEQ ID NO: 2970 represents the cDNA sequence for clone AA292280.

SEQ ID NO: 2971 represents the cDNA sequence for clone AA292289.

SEQ ID NO: 2972 represents the cDNA sequence for clone AA292292.

SEQ ID NO: 2973 represents the cDNA sequence for clone AA292293.

SEQ ID NO: 2974 represents the cDNA sequence for clone AA292297.

SEQ ID NO: 2975 represents the cDNA sequence for clone AA292301.

SEQ ID NO: 2976 represents the cDNA sequence for clone AA292305.

SEQ ID NO: 2977 represents the cDNA sequence for clone AA292192.

SEQ ID NO: 2978 represents the cDNA sequence for clone AA292259.

SEQ ID NO: 2979 represents the cDNA sequence for clone AA292307.

SEQ ID NO: 2980 represents the cDNA sequence for clone AA292308.

SEQ ID NO: 2981 represents the cDNA sequence for clone AA292348.

SEQ ID NO: 2982 represents the cDNA sequence for clone AA292352.

SEQ ID NO: 2983 represents the cDNA sequence for clone AA292356.

SEQ ID NO: 2984 represents the cDNA sequence for clone AA292357.

SEQ ID NO: 2985 represents the cDNA sequence for clone AA292359.

SEQ ID NO: 2986 represents the cDNA sequence for clone AA292363.

SEQ ID NO: 2987 represents the cDNA sequence for clone AA292364.

SEQ ID NO: 2988 represents the cDNA sequence for clone AA292366.

SEQ ID NO: 2989 represents the cDNA sequence for clone AA292367.

SEQ ID NO: 2990 represents the cDNA sequence for clone AA292370.

SEQ ID NO: 2991 represents the cDNA sequence for clone AA292376.

SEQ ID NO: 2992 represents the cDNA sequence for clone AA292223.

SEQ ID NO: 2993 represents the cDNA sequence for clone AA292227.

SEQ ID NO: 2994 represents the cDNA sequence for clone AA292331.

SEQ ID NO: 2995 represents the cDNA sequence for clone AA292382.

SEQ ID NO: 2996 represents the cDNA sequence for clone AA292383.

SEQ ID NO: 2997 represents the cDNA sequence for clone AA292403.

SEQ ID NO: 2998 represents the cDNA sequence for clone AA292405.

SEQ ID NO: 2999 represents the cDNA sequence for clone AA292411.

SEQ ID NO: 3000 represents the cDNA sequence for clone AA292415.

SEQ ID NO: 3001 represents the cDNA sequence for clone AA292418.

SEQ ID NO: 3002 represents the cDNA sequence for clone AA292420.

SEQ ID NO: 3003 represents the cDNA sequence for clone AA292423.

SEQ ID NO: 3004 represents the cDNA sequence for clone AA292435.

SEQ ID NO: 3005 represents the cDNA sequence for clone AA292436.

SEQ ID NO: 3006 represents the cDNA sequence for clone AA292254.

SEQ ID NO: 3007 represents the cDNA sequence for clone AA292394.

SEQ ID NO: 3008 represents the cDNA sequence for clone AA292395.

SEQ ID NO: 3009 represents the cDNA sequence for clone AA292396.

SEQ ID NO: 3010 represents the cDNA sequence for clone AA292399.

SEQ ID NO: 3011 represents the cDNA sequence for clone AA292387.

SEQ ID NO: 3012 represents the cDNA sequence for clone AA292388.

SEQ ID NO: 3013 represents the cDNA sequence for clone AA292389.

SEQ ID NO: 3014 represents the cDNA sequence for clone AA292454.

SEQ ID NO: 3015 represents the cDNA sequence for clone AA292473.

SEQ ID NO: 3016 represents the cDNA sequence for clone AA292475.

SEQ ID NO: 3017 represents the cDNA sequence for clone AA292447.

SEQ ID NO: 3018 represents the cDNA sequence for clone AA292449.

SEQ ID NO: 3019 represents the cDNA sequence for clone AA292486.

SEQ ID NO: 3020 represents the cDNA sequence for clone AA292487.

SEQ ID NO: 3021 represents the cDNA sequence for clone AA292505.

SEQ ID NO: 3022 represents the cDNA sequence for clone AA292508.

SEQ ID NO: 3023 represents the cDNA sequence for clone AA292316.

SEQ ID NO: 3024 represents the cDNA sequence for clone AA292319.

SEQ ID NO: 3025 represents the cDNA sequence for clone AA292516.

SEQ ID NO: 3026 represents the cDNA sequence for clone AA292543.

SEQ ID NO: 3027 represents the cDNA sequence for clone AA292549.

SEQ ID NO: 3028 represents the cDNA sequence for clone AA292564.

SEQ ID NO: 3029 represents the cDNA sequence for clone AA292520.

SEQ ID NO: 3030 represents the cDNA sequence for clone AA292527.

SEQ ID NO: 3031 represents the cDNA sequence for clone AA292574.

SEQ ID NO: 3032 represents the cDNA sequence for clone AA292575.

SEQ ID NO: 3033 represents the cDNA sequence for clone AA292576.

SEQ ID NO: 3034 represents the cDNA sequence for clone AA292579.

SEQ ID NO: 3035 represents the cDNA sequence for clone AA292465.

SEQ ID NO: 3036 represents the cDNA sequence for clone AA292567.

SEQ ID NO: 3037 represents the cDNA sequence for clone AA292569.

SEQ ID NO: 3038 represents the cDNA sequence for clone AA292584.

SEQ ID NO: 3039 represents the cDNA sequence for clone AA292587.

SEQ ID NO: 3040 represents the cDNA sequence for clone AA292588.

SEQ ID NO: 3041 represents the cDNA sequence for clone AA292589.

SEQ ID NO: 3042 represents the cDNA sequence for clone AA292600.

SEQ ID NO: 3043 represents the cDNA sequence for clone AA292602.

SEQ ID NO: 3044 represents the cDNA sequence for clone AA292604.

SEQ ID NO: 3045 represents the cDNA sequence for clone AA292645.

SEQ ID NO: 3046 represents the cDNA sequence for clone AA292650.

SEQ ID NO: 3047 represents the cDNA sequence for clone AA292663.

SEQ ID NO: 3048 represents the cDNA sequence for clone AA292669.

SEQ ID NO: 3049 represents the cDNA sequence for clone AA292677.

SEQ ID NO: 3050 represents the cDNA sequence for clone AA292684.

SEQ ID NO: 3051 represents the cDNA sequence for clone AA292689.

SEQ ID NO: 3052 represents the cDNA sequence for clone AA293328.

SEQ ID NO: 3053 represents the cDNA sequence for clone AA293352.

SEQ ID NO: 3054 represents the cDNA sequence for clone AA293355.

SEQ ID NO: 3055 represents the cDNA sequence for clone AA293360.

SEQ ID NO: 3056 represents the cDNA sequence for clone AA293361.

SEQ ID NO: 3057 represents the cDNA sequence for clone AA293364.

SEQ ID NO: 3058 represents the cDNA sequence for clone AA293332.

SEQ ID NO: 3059 represents the cDNA sequence for clone AA293333.

SEQ ID NO: 3060 represents the cDNA sequence for clone AA293383.

SEQ ID NO: 3061 represents the cDNA sequence for clone AA293384.

SEQ ID NO: 3062 represents the cDNA sequence for clone AA293385.

SEQ ID NO: 3063 represents the cDNA sequence for clone AA293387.

SEQ ID NO: 3064 represents the cDNA sequence for clone AA293399.

SEQ ID NO: 3065 represents the cDNA sequence for clone AA293401.

SEQ ID NO: 3066 represents the cDNA sequence for clone AA293403.

SEQ ID NO: 3067 represents the cDNA sequence for clone AA293404.

SEQ ID NO: 3068 represents the cDNA sequence for clone AA293409.

SEQ ID NO: 3069 represents the cDNA sequence for clone AA293413.

SEQ ID NO: 3070 represents the cDNA sequence for clone AA293414.

SEQ ID NO: 3071 represents the cDNA sequence for clone AA293422.

SEQ ID NO: 3072 represents the cDNA sequence for clone AA293426.

SEQ ID NO: 3073 represents the cDNA sequence for clone AA293427.

SEQ ID NO: 3074 represents the cDNA sequence for clone AA293435.

SEQ ID NO: 3075 represents the cDNA sequence for clone AA293440.

SEQ ID NO: 3076 represents the cDNA sequence for clone AA293366.

SEQ ID NO: 3077 represents the cDNA sequence for clone AA293369.

SEQ ID NO: 3078 represents the cDNA sequence for clone AA292955.

SEQ ID NO: 3079 represents the cDNA sequence for clone AA292961.

SEQ ID NO: 3080 represents the cDNA sequence for clone AA292962.

SEQ ID NO: 3081 represents the cDNA sequence for clone AA292965.

SEQ ID NO: 3082 represents the cDNA sequence for clone AA292972.

SEQ ID NO: 3083 represents the cDNA sequence for clone AA292975.

SEQ ID NO: 3084 represents the cDNA sequence for clone AA292977.

SEQ ID NO: 3085 represents the cDNA sequence for clone AA292942.

SEQ ID NO: 3086 represents the cDNA sequence for clone AA293373.

SEQ ID NO: 3087 represents the cDNA sequence for clone AA293375.

SEQ ID NO: 3088 represents the cDNA sequence for clone AA293376.

SEQ ID NO: 3089 represents the cDNA sequence for clone AA293378.

SEQ ID NO: 3090 represents the cDNA sequence for clone AA293379.

SEQ ID NO: 3091 represents the cDNA sequence for clone AA293380.

SEQ ID NO: 3092 represents the cDNA sequence for clone AA293381.

SEQ ID NO: 3093 represents the cDNA sequence for clone AA292986.

SEQ ID NO: 3094 represents the cDNA sequence for clone AA292987.

SEQ ID NO: 3095 represents the cDNA sequence for clone AA292988.

SEQ ID NO: 3096 represents the cDNA sequence for clone AA292993.

SEQ ID NO: 3097 represents the cDNA sequence for clone AA292994.

SEQ ID NO: 3098 represents the cDNA sequence for clone AA292998.

SEQ ID NO: 3099 represents the cDNA sequence for clone AA293001.

SEQ ID NO: 3100 represents the cDNA sequence for clone AA293029.

SEQ ID NO: 3101 represents the cDNA sequence for clone AA293034.

SEQ ID NO: 3102 represents the cDNA sequence for clone AA293035.

SEQ ID NO: 3103 represents the cDNA sequence for clone AA293036.

SEQ ID NO: 3104 represents the cDNA sequence for clone AA293043.

SEQ ID NO: 3105 represents the cDNA sequence for clone AA293044.

SEQ ID NO: 3106 represents the cDNA sequence for clone AA293049.

SEQ ID NO: 3107 represents the cDNA sequence for clone AA293051.

SEQ ID NO: 3108 represents the cDNA sequence for clone AA293054.

SEQ ID NO: 3109 represents the cDNA sequence for clone AA292944.

SEQ ID NO: 3110 represents the cDNA sequence for clone AA292948.

SEQ ID NO: 3111 represents the cDNA sequence for clone AA293394.

SEQ ID NO: 3112 represents the cDNA sequence for clone AA293395.

SEQ ID NO: 3113 represents the cDNA sequence for clone AA293443.

SEQ ID NO: 3114 represents the cDNA sequence for clone AA293471.

SEQ ID NO: 3115 represents the cDNA sequence for clone AA293472.

SEQ ID NO: 3116 represents the cDNA sequence for clone AA293479.

SEQ ID NO: 3117 represents the cDNA sequence for clone AA293485.

SEQ ID NO: 3118 represents the cDNA sequence for clone AA293496.

SEQ ID NO: 3119 represents the cDNA sequence for clone AA293008.

SEQ ID NO: 3120 represents the cDNA sequence for clone AA293458.

SEQ ID NO: 3121 represents the cDNA sequence for clone AA293460.

SEQ ID NO: 3122 represents the cDNA sequence for clone AA293466.

SEQ ID NO: 3123 represents the cDNA sequence for clone AA293502.

SEQ ID NO: 3124 represents the cDNA sequence for clone AA293507.

SEQ ID NO: 3125 represents the cDNA sequence for clone AA293532.

SEQ ID NO: 3126 represents the cDNA sequence for clone AA293533.

SEQ ID NO: 3127 represents the cDNA sequence for clone AA293534.

SEQ ID NO: 3128 represents the cDNA sequence for clone AA293542.

SEQ ID NO: 3129 represents the cDNA sequence for clone AA293548.

SEQ ID NO: 3130 represents the cDNA sequence for clone AA293549.

SEQ ID NO: 3131 represents the cDNA sequence for clone AA293510.

SEQ ID NO: 3132 represents the cDNA sequence for clone AA293512.

SEQ ID NO: 3133 represents the cDNA sequence for clone AA293016.

SEQ ID NO: 3134 represents the cDNA sequence for clone AA293017.

SEQ ID NO: 3135 represents the cDNA sequence for clone AA293018.

SEQ ID NO: 3136 represents the cDNA sequence for clone AA293088.

SEQ ID NO: 3137 represents the cDNA sequence for clone AA293070.

SEQ ID NO: 3138 represents the cDNA sequence for clone AA293072.

SEQ ID NO: 3139 represents the cDNA sequence for clone AA293109.

SEQ ID NO: 3140 represents the cDNA sequence for clone AA293119.

SEQ ID NO: 3141 represents the cDNA sequence for clone AA293451.

SEQ ID NO: 3142 represents the cDNA sequence for clone AA293095.

SEQ ID NO: 3143 represents the cDNA sequence for clone AA293099.

SEQ ID NO: 3144 represents the cDNA sequence for clone AA293159.

SEQ ID NO: 3145 represents the cDNA sequence for clone AA293164.

SEQ ID NO: 3146 represents the cDNA sequence for clone AA293179.

SEQ ID NO: 3147 represents the cDNA sequence for clone AA293183.

SEQ ID NO: 3148 represents the cDNA sequence for clone AA293193.

SEQ ID NO: 3149 represents the cDNA sequence for clone AA293194.

SEQ ID NO: 3150 represents the cDNA sequence for clone AA293081.

SEQ ID NO: 3151 represents the cDNA sequence for clone AA293083.

SEQ ID NO: 3152 represents the cDNA sequence for clone AA293517.

SEQ ID NO: 3153 represents the cDNA sequence for clone AA293518.

SEQ ID NO: 3154 represents the cDNA sequence for clone AA293521.

SEQ ID NO: 3155 represents the cDNA sequence for clone AA293523.

SEQ ID NO: 3156 represents the cDNA sequence for clone AA293570.

SEQ ID NO: 3157 represents the cDNA sequence for clone AA293574.

SEQ ID NO: 3158 represents the cDNA sequence for clone AA293593.

SEQ ID NO: 3159 represents the cDNA sequence for clone AA293595.

SEQ ID NO: 3160 represents the cDNA sequence for clone AA293596.

SEQ ID NO: 3161 represents the cDNA sequence for clone AA293597.

SEQ ID NO: 3162 represents the cDNA sequence for clone AA293599.

SEQ ID NO: 3163 represents the cDNA sequence for clone AA293600.

SEQ ID NO: 3164 represents the cDNA sequence for clone AA293601.

SEQ ID NO: 3165 represents the cDNA sequence for clone AA293628.

SEQ ID NO: 3166 represents the cDNA sequence for clone AA293632.

SEQ ID NO: 3167 represents the cDNA sequence for clone AA293138.

SEQ ID NO: 3168 represents the cDNA sequence for clone AA293145.

SEQ ID NO: 3169 represents the cDNA sequence for clone AA293147.

SEQ ID NO: 3170 represents the cDNA sequence for clone AA293197.

SEQ ID NO: 3171 represents the cDNA sequence for clone AA293200.

SEQ ID NO: 3172 represents the cDNA sequence for clone AA293202.

SEQ ID NO: 3173 represents the cDNA sequence for clone AA293221.

SEQ ID NO: 3174 represents the cDNA sequence for clone AA293222.

SEQ ID NO: 3175 represents the cDNA sequence for clone AA293223.

SEQ ID NO: 3176 represents the cDNA sequence for clone AA293226.

SEQ ID NO: 3177 represents the cDNA sequence for clone AA293228.

SEQ ID NO: 3178 represents the cDNA sequence for clone AA293240.

SEQ ID NO: 3179 represents the cDNA sequence for clone AA293242.

SEQ ID NO: 3180 represents the cDNA sequence for clone AA293243.

SEQ ID NO: 3181 represents the cDNA sequence for clone AA293248.

SEQ ID NO: 3182 represents the cDNA sequence for clone AA293249.

SEQ ID NO: 3183 represents the cDNA sequence for clone AA293252.

SEQ ID NO: 3184 represents the cDNA sequence for clone AA293254.

SEQ ID NO: 3185 represents the cDNA sequence for clone AA293257.

SEQ ID NO: 3186 represents the cDNA sequence for clone AA293258.

SEQ ID NO: 3187 represents the cDNA sequence for clone AA293219.

SEQ ID NO: 3188 represents the cDNA sequence for clone AA293295.

SEQ ID NO: 3189 represents the cDNA sequence for clone AA293302.

SEQ ID NO: 3190 represents the cDNA sequence for clone AA293304.

SEQ ID NO: 3191 represents the cDNA sequence for clone AA293320.

SEQ ID NO: 3192 represents the cDNA sequence for clone AA293326.

SEQ ID NO: 3193 represents the cDNA sequence for clone AA293270.

SEQ ID NO: 3194 represents the cDNA sequence for clone AA293275.

SEQ ID NO: 3195 represents the cDNA sequence for clone AA293662.

SEQ ID NO: 3196 represents the cDNA sequence for clone AA293673.

SEQ ID NO: 3197 represents the cDNA sequence for clone AA293678.

SEQ ID NO: 3198 represents the cDNA sequence for clone AA293685.

SEQ ID NO: 3199 represents the cDNA sequence for clone AA293689.

SEQ ID NO: 3200 represents the cDNA sequence for clone AA293691.

SEQ ID NO: 3201 represents the cDNA sequence for clone AA293700.

SEQ ID NO: 3202 represents the cDNA sequence for clone AA293205.

SEQ ID NO: 3203 represents the cDNA sequence for clone AA293655.

SEQ ID NO: 3204 represents the cDNA sequence for clone AA293656.

SEQ ID NO: 3205 represents the cDNA sequence for clone AA293658.

SEQ ID NO: 3206 represents the cDNA sequence for clone AA293659.

SEQ ID NO: 3207 represents the cDNA sequence for clone AA293727.

SEQ ID NO: 3208 represents the cDNA sequence for clone AA293737.

SEQ ID NO: 3209 represents the cDNA sequence for clone AA293742.

SEQ ID NO: 3210 represents the cDNA sequence for clone AA293749.

SEQ ID NO: 3211 represents the cDNA sequence for clone AA293753.

SEQ ID NO: 3212 represents the cDNA sequence for clone AA293761.

SEQ ID NO: 3213 represents the cDNA sequence for clone AA293650.

SEQ ID NO: 3214 represents the cDNA sequence for clone AA293280.

SEQ ID NO: 3215 represents the cDNA sequence for clone AA292730.

SEQ ID NO: 3216 represents the cDNA sequence for clone AA292736.

SEQ ID NO: 3217 represents the cDNA sequence for clone AA292759.

SEQ ID NO: 3218 represents the cDNA sequence for clone AA292765.

SEQ ID NO: 3219 represents the cDNA sequence for clone AA292770.

SEQ ID NO: 3220 represents the cDNA sequence for clone AA292778.

SEQ ID NO: 3221 represents the cDNA sequence for clone AA292779.

SEQ ID NO: 3222 represents the cDNA sequence for clone AA292789.

SEQ ID NO: 3223 represents the cDNA sequence for clone AA292747.

SEQ ID NO: 3224 represents the cDNA sequence for clone AA292868.

SEQ ID NO: 3225 represents the cDNA sequence for clone AA292869.

SEQ ID NO: 3226 represents the cDNA sequence for clone AA293771.

SEQ ID NO: 3227 represents the cDNA sequence for clone AA293772.

SEQ ID NO: 3228 represents the cDNA sequence for clone AA293773.

SEQ ID NO: 3229 represents the cDNA sequence for clone AA293775.

SEQ ID NO: 3230 represents the cDNA sequence for clone AA293795.

SEQ ID NO: 3231 represents the cDNA sequence for clone AA394039.

SEQ ID NO: 3232 represents the cDNA sequence for clone AA394066.

SEQ ID NO: 3233 represents the cDNA sequence for clone AA394069.

SEQ ID NO: 3234 represents the cDNA sequence for clone AA394040.

SEQ ID NO: 3235 represents the cDNA sequence for clone AA394041.

SEQ ID NO: 3236 represents the cDNA sequence for clone AA394044.

SEQ ID NO: 3237 represents the cDNA sequence for clone AA394084.

SEQ ID NO: 3238 represents the cDNA sequence for clone AA394086.

SEQ ID NO: 3239 represents the cDNA sequence for clone AA394090.

SEQ ID NO: 3240 represents the cDNA sequence for clone AA394091.

SEQ ID NO: 3241 represents the cDNA sequence for clone AA394092.

SEQ ID NO: 3242 represents the cDNA sequence for clone AA394133.

SEQ ID NO: 3243 represents the cDNA sequence for clone AA394135.

SEQ ID NO: 3244 represents the cDNA sequence for clone AA394141.

SEQ ID NO: 3245 represents the cDNA sequence for clone AA394145.

SEQ ID NO: 3246 represents the cDNA sequence for clone AA394147.

SEQ ID NO: 3247 represents the cDNA sequence for clone AA394108.

SEQ ID NO: 3248 represents the cDNA sequence for clone AA394166.

SEQ ID NO: 3249 represents the cDNA sequence for clone AA394124.

SEQ ID NO: 3250 represents the cDNA sequence for clone AA394052.

SEQ ID NO: 3251 represents the cDNA sequence for clone AA394056.

SEQ ID NO: 3252 represents the cDNA sequence for clone AA394170.

SEQ ID NO: 3253 represents the cDNA sequence for clone AA394174.

SEQ ID NO: 3254 represents the cDNA sequence for clone AA394180.

SEQ ID NO: 3255 represents the cDNA sequence for clone AA394182.

SEQ ID NO: 3256 represents the cDNA sequence for clone AA394233.

SEQ ID NO: 3257 represents the cDNA sequence for clone AA394236.

SEQ ID NO: 3258 represents the cDNA sequence for clone AA393998.

SEQ ID NO: 3259 represents the cDNA sequence for clone AA393999.

SEQ ID NO: 3260 represents the cDNA sequence for clone AA394003.

SEQ ID NO: 3261 represents the cDNA sequence for clone AA394195.

SEQ ID NO: 3262 represents the cDNA sequence for clone AA394197.

SEQ ID NO: 3263 represents the cDNA sequence for clone AA394200.

SEQ ID NO: 3264 represents the cDNA sequence for clone AA394241.

SEQ ID NO: 3265 represents the cDNA sequence for clone AA394251.

SEQ ID NO: 3266 represents the cDNA sequence for clone AA394259.

SEQ ID NO: 3267 represents the cDNA sequence for clone AA394261.

SEQ ID NO: 3268 represents the cDNA sequence for clone AA394264.

SEQ ID NO: 3269 represents the cDNA sequence for clone AA394268.

SEQ ID NO: 3270 represents the cDNA sequence for clone AA394276.

SEQ ID NO: 3271 represents the cDNA sequence for clone AA394278.

SEQ ID NO: 3272 represents the cDNA sequence for clone AA394281.

SEQ ID NO: 3273 represents the cDNA sequence for clone AA397815.

SEQ ID NO: 3274 represents the cDNA sequence for clone AA397821.

SEQ ID NO: 3275 represents the cDNA sequence for clone AA397822.

SEQ ID NO: 3276 represents the cDNA sequence for clone AA397823.

SEQ ID NO: 3277 represents the cDNA sequence for clone AA397827.

SEQ ID NO: 3278 represents the cDNA sequence for clone AA398314.

SEQ ID NO: 3279 represents the cDNA sequence for clone AA398451.

SEQ ID NO: 3280 represents the cDNA sequence for clone AA398453.

SEQ ID NO: 3281 represents the cDNA sequence for clone AA398523.

SEQ ID NO: 3282 represents the cDNA sequence for clone AA398579.

SEQ ID NO: 3283 represents the cDNA sequence for clone AA298089.

SEQ ID NO: 3284 represents the cDNA sequence for clone AA297920.

SEQ ID NO: 3285 represents the cDNA sequence for clone AA297922.

SEQ ID NO: 3286 represents the cDNA sequence for clone AA298143.

SEQ ID NO: 3287 represents the cDNA sequence for clone AA362635.

SEQ ID NO: 3288 represents the cDNA sequence for clone AA362636.

SEQ ID NO: 3289 represents the cDNA sequence for clone AA362637.

SEQ ID NO: 3290 represents the cDNA sequence for clone AA362639.

SEQ ID NO: 3291 represents the cDNA sequence for clone AA362641.

SEQ ID NO: 3292 represents the cDNA sequence for clone AA362693.

SEQ ID NO: 3293 represents the cDNA sequence for clone AA362694.

SEQ ID NO: 3294 represents the cDNA sequence for clone AA362696.

SEQ ID NO: 3295 represents the cDNA sequence for clone AA362697.

SEQ ID NO: 3296 represents the cDNA sequence for clone AA362698.

SEQ ID NO: 3297 represents the cDNA sequence for clone AA362707.

SEQ ID NO: 3298 represents the cDNA sequence for clone AA362710.

SEQ ID NO: 3299 represents the cDNA sequence for clone AA362711.

SEQ ID NO: 3300 represents the cDNA sequence for clone AA362733.

SEQ ID NO: 3301 represents the cDNA sequence for clone AA362734.

SEQ ID NO: 3302 represents the cDNA sequence for clone AA362736.

SEQ ID NO: 3303 represents the cDNA sequence for clone AA362740.

SEQ ID NO: 3304 represents the cDNA sequence for clone AA362742.

SEQ ID NO: 3305 represents the cDNA sequence for clone AA362743.

SEQ ID NO: 3306 represents the cDNA sequence for clone AA362744.

SEQ ID NO: 3307 represents the cDNA sequence for clone AA362745.

SEQ ID NO: 3308 represents the cDNA sequence for clone AA362747.

SEQ ID NO: 3309 represents the cDNA sequence for clone AA362748.

SEQ ID NO: 3310 represents the cDNA sequence for clone AA362750.

SEQ ID NO: 3311 represents the cDNA sequence for clone AA362751.

SEQ ID NO: 3312 represents the cDNA sequence for clone AA362753.

SEQ ID NO: 3313 represents the cDNA sequence for clone AA362754.

SEQ ID NO: 3314 represents the cDNA sequence for clone AA362759.

SEQ ID NO: 3315 represents the cDNA sequence for clone AA362761.

SEQ ID NO: 3316 represents the cDNA sequence for clone AA362762.

SEQ ID NO: 3317 represents the cDNA sequence for clone AA362763.

SEQ ID NO: 3318 represents the cDNA sequence for clone AA362591.

SEQ ID NO: 3319 represents the cDNA sequence for clone AA362593.

SEQ ID NO: 3320 represents the cDNA sequence for clone AA362595.

SEQ ID NO: 3321 represents the cDNA sequence for clone AA362596.

SEQ ID NO: 3322 represents the cDNA sequence for clone AA362598.

SEQ ID NO: 3323 represents the cDNA sequence for clone AA362723.

SEQ ID NO: 3324 represents the cDNA sequence for clone AA362726.

SEQ ID NO: 3325 represents the cDNA sequence for clone AA362728.

SEQ ID NO: 3326 represents the cDNA sequence for clone AA362732.

SEQ ID NO: 3327 represents the cDNA sequence for clone AA362764.

SEQ ID NO: 3328 represents the cDNA sequence for clone AA362766.

SEQ ID NO: 3329 represents the cDNA sequence for clone AA362767.

SEQ ID NO: 3330 represents the cDNA sequence for clone AA362770.

SEQ ID NO: 3331 represents the cDNA sequence for clone AA362773.

SEQ ID NO: 3332 represents the cDNA sequence for clone AA362774.

SEQ ID NO: 3333 represents the cDNA sequence for clone AA362796.

SEQ ID NO: 3334 represents the cDNA sequence for clone AA362797.

SEQ ID NO: 3335 represents the cDNA sequence for clone AA362800.

SEQ ID NO: 3336 represents the cDNA sequence for clone AA362802.

SEQ ID NO: 3337 represents the cDNA sequence for clone AA362806.

SEQ ID NO: 3338 represents the cDNA sequence for clone AA362808.

SEQ ID NO: 3339 represents the cDNA sequence for clone AA362809.

SEQ ID NO: 3340 represents the cDNA sequence for clone AA362810.

SEQ ID NO: 3341 represents the cDNA sequence for clone AA362811.

SEQ ID NO: 3342 represents the cDNA sequence for clone AA362812.

SEQ ID NO: 3343 represents the cDNA sequence for clone AA362816.

SEQ ID NO: 3344 represents the cDNA sequence for clone AA362818.

SEQ ID NO: 3345 represents the cDNA sequence for clone AA362821.

SEQ ID NO: 3346 represents the cDNA sequence for clone AA362822.

SEQ ID NO: 3347 represents the cDNA sequence for clone AA362824.

SEQ ID NO: 3348 represents the cDNA sequence for clone AA362826.

SEQ ID NO: 3349 represents the cDNA sequence for clone AA362830.

SEQ ID NO: 3350 represents the cDNA sequence for clone AA362831.

SEQ ID NO: 3351 represents the cDNA sequence for clone AA362832.

SEQ ID NO: 3352 represents the cDNA sequence for clone AA362834.

SEQ ID NO: 3353 represents the cDNA sequence for clone AA362840.

SEQ ID NO: 3354 represents the cDNA sequence for clone AA362841.

SEQ ID NO: 3355 represents the cDNA sequence for clone AA362842.

SEQ ID NO: 3356 represents the cDNA sequence for clone AA362844.

SEQ ID NO: 3357 represents the cDNA sequence for clone AA362845.

SEQ ID NO: 3358 represents the cDNA sequence for clone AA362775.

SEQ ID NO: 3359 represents the cDNA sequence for clone AA362778.

SEQ ID NO: 3360 represents the cDNA sequence for clone AA362780.

SEQ ID NO: 3361 represents the cDNA sequence for clone AA362781.

SEQ ID NO: 3362 represents the cDNA sequence for clone AA362782.

SEQ ID NO: 3363 represents the cDNA sequence for clone AA362784.

SEQ ID NO: 3364 represents the cDNA sequence for clone AA362785.

SEQ ID NO: 3365 represents the cDNA sequence for clone AA362627.

SEQ ID NO: 3366 represents the cDNA sequence for clone AA362629.

SEQ ID NO: 3367 represents the cDNA sequence for clone AA362849.

SEQ ID NO: 3368 represents the cDNA sequence for clone AA362850.

SEQ ID NO: 3369 represents the cDNA sequence for clone AA362851.

SEQ ID NO: 3370 represents the cDNA sequence for clone AA362854.

SEQ ID NO: 3371 represents the cDNA sequence for clone AA362856.

SEQ ID NO: 3372 represents the cDNA sequence for clone AA362879.

SEQ ID NO: 3373 represents the cDNA sequence for clone AA362881.

SEQ ID NO: 3374 represents the cDNA sequence for clone AA362884.

SEQ ID NO: 3375 represents the cDNA sequence for clone AA362887.

SEQ ID NO: 3376 represents the cDNA sequence for clone AA362889.

SEQ ID NO: 3377 represents the cDNA sequence for clone AA362892.

SEQ ID NO: 3378 represents the cDNA sequence for clone AA362895.

SEQ ID NO: 3379 represents the cDNA sequence for clone AA362898.

SEQ ID NO: 3380 represents the cDNA sequence for clone AA362899.

SEQ ID NO: 3381 represents the cDNA sequence for clone AA362903.

SEQ ID NO: 3382 represents the cDNA sequence for clone AA362906.

SEQ ID NO: 3383 represents the cDNA sequence for clone AA362907.

SEQ ID NO: 3384 represents the cDNA sequence for clone AA362908.

SEQ ID NO: 3385 represents the cDNA sequence for clone AA362910.

SEQ ID NO: 3386 represents the cDNA sequence for clone AA362911.

SEQ ID NO: 3387 represents the cDNA sequence for clone AA362912.

SEQ ID NO: 3388 represents the cDNA sequence for clone AA362913.

SEQ ID NO: 3389 represents the cDNA sequence for clone AA362918.

SEQ ID NO: 3390 represents the cDNA sequence for clone AA362919.

SEQ ID NO: 3391 represents the cDNA sequence for clone AA362921.

SEQ ID NO: 3392 represents the cDNA sequence for clone AA362924.

SEQ ID NO: 3393 represents the cDNA sequence for clone AA362927.

SEQ ID NO: 3394 represents the cDNA sequence for clone AA362928.

SEQ ID NO: 3395 represents the cDNA sequence for clone AA362929.

SEQ ID NO: 3396 represents the cDNA sequence for clone AA362930.

SEQ ID NO: 3397 represents the cDNA sequence for clone AA362931.

SEQ ID NO: 3398 represents the cDNA sequence for clone AA362714.

SEQ ID NO: 3399 represents the cDNA sequence for clone AA362717.

SEQ ID NO: 3400 represents the cDNA sequence for clone AA362719.

SEQ ID NO: 3401 represents the cDNA sequence for clone AA362871.

SEQ ID NO: 3402 represents the cDNA sequence for clone AA362872.

SEQ ID NO: 3403 represents the cDNA sequence for clone AA362876.

SEQ ID NO: 3404 represents the cDNA sequence for clone AA362877.

SEQ ID NO: 3405 represents the cDNA sequence for clone AA362878.

SEQ ID NO: 3406 represents the cDNA sequence for clone AA362934.

SEQ ID NO: 3407 represents the cDNA sequence for clone AA362937.

SEQ ID NO: 3408 represents the cDNA sequence for clone AA362941.

SEQ ID NO: 3409 represents the cDNA sequence for clone AA362942.

SEQ ID NO: 3410 represents the cDNA sequence for clone AA362968.

SEQ ID NO: 3411 represents the cDNA sequence for clone AA362969.

SEQ ID NO: 3412 represents the cDNA sequence for clone AA362970.

SEQ ID NO: 3413 represents the cDNA sequence for clone AA362971.

SEQ ID NO: 3414 represents the cDNA sequence for clone AA362972.

SEQ ID NO: 3415 represents the cDNA sequence for clone AA362973.

SEQ ID NO: 3416 represents the cDNA sequence for clone AA362985.

SEQ ID NO: 3417 represents the cDNA sequence for clone AA362988.

SEQ ID NO: 3418 represents the cDNA sequence for clone AA362989.

SEQ ID NO: 3419 represents the cDNA sequence for clone AA362993.

SEQ ID NO: 3420 represents the cDNA sequence for clone AA362994.

SEQ ID NO: 3421 represents the cDNA sequence for clone AA363001.

SEQ ID NO: 3422 represents the cDNA sequence for clone AA363006.

SEQ ID NO: 3423 represents the cDNA sequence for clone AA363009.

SEQ ID NO: 3424 represents the cDNA sequence for clone AA363010.

SEQ ID NO: 3425 represents the cDNA sequence for clone AA363011.

SEQ ID NO: 3426 represents the cDNA sequence for clone AA363012.

SEQ ID NO: 3427 represents the cDNA sequence for clone AA363013.

SEQ ID NO: 3428 represents the cDNA sequence for clone AA363014.

SEQ ID NO: 3429 represents the cDNA sequence for clone AA362786.

SEQ ID NO: 3430 represents the cDNA sequence for clone AA362788.

SEQ ID NO: 3431 represents the cDNA sequence for clone AA362790.

SEQ ID NO: 3432 represents the cDNA sequence for clone AA362791.

SEQ ID NO: 3433 represents the cDNA sequence for clone AA362961.

SEQ ID NO: 3434 represents the cDNA sequence for clone AA362962.

SEQ ID NO: 3435 represents the cDNA sequence for clone AA362963.

SEQ ID NO: 3436 represents the cDNA sequence for clone AA363023.

SEQ ID NO: 3437 represents the cDNA sequence for clone AA363048.

SEQ ID NO: 3438 represents the cDNA sequence for clone AA363050.

SEQ ID NO: 3439 represents the cDNA sequence for clone AA363052.

SEQ ID NO: 3440 represents the cDNA sequence for clone AA363053.

SEQ ID NO: 3441 represents the cDNA sequence for clone AA363055.

SEQ ID NO: 3442 represents the cDNA sequence for clone AA363062.

SEQ ID NO: 3443 represents the cDNA sequence for clone AA363064.

SEQ ID NO: 3444 represents the cDNA sequence for clone AA363070.

SEQ ID NO: 3445 represents the cDNA sequence for clone AA363073.

SEQ ID NO: 3446 represents the cDNA sequence for clone AA363074.

SEQ ID NO: 3447 represents the cDNA sequence for clone AA363075.

SEQ ID NO: 3448 represents the cDNA sequence for clone AA363076.

SEQ ID NO: 3449 represents the cDNA sequence for clone AA363082.

SEQ ID NO: 3450 represents the cDNA sequence for clone AA363089.

SEQ ID NO: 3451 represents the cDNA sequence for clone AA363092.

SEQ ID NO: 3452 represents the cDNA sequence for clone AA362858.

SEQ ID NO: 3453 represents the cDNA sequence for clone AA362859.

SEQ ID NO: 3454 represents the cDNA sequence for clone AA362862.

SEQ ID NO: 3455 represents the cDNA sequence for clone AA362863.

SEQ ID NO: 3456 represents the cDNA sequence for clone AA362865.

SEQ ID NO: 3457 represents the cDNA sequence for clone AA362866.

SEQ ID NO: 3458 represents the cDNA sequence for clone AA362867.

SEQ ID NO: 3459 represents the cDNA sequence for clone AA363041.

SEQ ID NO: 3460 represents the cDNA sequence for clone AA363042.

SEQ ID NO: 3461 represents the cDNA sequence for clone AA363043.

SEQ ID NO: 3462 represents the cDNA sequence for clone AA363044.

SEQ ID NO: 3463 represents the cDNA sequence for clone AA363046.

SEQ ID NO: 3464 represents the cDNA sequence for clone AA363099.

SEQ ID NO: 3465 represents the cDNA sequence for clone AA363104.

SEQ ID NO: 3466 represents the cDNA sequence for clone AA363105.

SEQ ID NO: 3467 represents the cDNA sequence for clone AA363106.

SEQ ID NO: 3468 represents the cDNA sequence for clone AA363107.

SEQ ID NO: 3469 represents the cDNA sequence for clone AA363131.

SEQ ID NO: 3470 represents the cDNA sequence for clone AA363134.

SEQ ID NO: 3471 represents the cDNA sequence for clone AA363136.

SEQ ID NO: 3472 represents the cDNA sequence for clone AA363137.

SEQ ID NO: 3473 represents the cDNA sequence for clone AA363139.

SEQ ID NO: 3474 represents the cDNA sequence for clone AA363141.

SEQ ID NO: 3475 represents the cDNA sequence for clone AA363146.

SEQ ID NO: 3476 represents the cDNA sequence for clone AA363148.

SEQ ID NO: 3477 represents the cDNA sequence for clone AA363150.

SEQ ID NO: 3478 represents the cDNA sequence for clone AA363151.

SEQ ID NO: 3479 represents the cDNA sequence for clone AA363152.

SEQ ID NO: 3480 represents the cDNA sequence for clone AA363154.

SEQ ID NO: 3481 represents the cDNA sequence for clone AA363155.

SEQ ID NO: 3482 represents the cDNA sequence for clone AA363156.

SEQ ID NO: 3483 represents the cDNA sequence for clone AA363160.

SEQ ID NO: 3484 represents the cDNA sequence for clone AA363161.

SEQ ID NO: 3485 represents the cDNA sequence for clone AA363164.

SEQ ID NO: 3486 represents the cDNA sequence for clone AA363165.

SEQ ID NO: 3487 represents the cDNA sequence for clone AA363166.

SEQ ID NO: 3488 represents the cDNA sequence for clone AA363169.

SEQ ID NO: 3489 represents the cDNA sequence for clone AA363171.

SEQ ID NO: 3490 represents the cDNA sequence for clone AA363172.

SEQ ID NO: 3491 represents the cDNA sequence for clone AA363174.

SEQ ID NO: 3492 represents the cDNA sequence for clone AA363177.

SEQ ID NO: 3493 represents the cDNA sequence for clone AA363179.

SEQ ID NO: 3494 represents the cDNA sequence for clone AA363181.

SEQ ID NO: 3495 represents the cDNA sequence for clone AA363183.

SEQ ID NO: 3496 represents the cDNA sequence for clone AA362946.

SEQ ID NO: 3497 represents the cDNA sequence for clone AA362947.

SEQ ID NO: 3498 represents the cDNA sequence for clone AA362949.

SEQ ID NO: 3499 represents the cDNA sequence for clone AA362950.

SEQ ID NO: 3500 represents the cDNA sequence for clone AA362951.

SEQ ID NO: 3501 represents the cDNA sequence for clone AA362953.

SEQ ID NO: 3502 represents the cDNA sequence for clone AA363119.

SEQ ID NO: 3503 represents the cDNA sequence for clone AA363120.

SEQ ID NO: 3504 represents the cDNA sequence for clone AA363121.

SEQ ID NO: 3505 represents the cDNA sequence for clone AA363122.

SEQ ID NO: 3506 represents the cDNA sequence for clone AA363123.

SEQ ID NO: 3507 represents the cDNA sequence for clone AA363124.

SEQ ID NO: 3508 represents the cDNA sequence for clone AA363127.

SEQ ID NO: 3509 represents the cDNA sequence for clone AA363129.

SEQ ID NO: 3510 represents the cDNA sequence for clone AA363186.

SEQ ID NO: 3511 represents the cDNA sequence for clone AA363187.

SEQ ID NO: 3512 represents the cDNA sequence for clone AA363190.

SEQ ID NO: 3513 represents the cDNA sequence for clone AA363215.

SEQ ID NO: 3514 represents the cDNA sequence for clone AA363217.

SEQ ID NO: 3515 represents the cDNA sequence for clone AA363221.

SEQ ID NO: 3516 represents the cDNA sequence for clone AA363245.

SEQ ID NO: 3517 represents the cDNA sequence for clone AA363246.

SEQ ID NO: 3518 represents the cDNA sequence for clone AA363247.

SEQ ID NO: 3519 represents the cDNA sequence for clone AA363250.

SEQ ID NO: 3520 represents the cDNA sequence for clone AA363251.

SEQ ID NO: 3521 represents the cDNA sequence for clone AA363259.

SEQ ID NO: 3522 represents the cDNA sequence for clone AA363260.

SEQ ID NO: 3523 represents the cDNA sequence for clone AA363261.

SEQ ID NO: 3524 represents the cDNA sequence for clone AA363028.

SEQ ID NO: 3525 represents the cDNA sequence for clone AA363033.

SEQ ID NO: 3526 represents the cDNA sequence for clone AA363225.

SEQ ID NO: 3527 represents the cDNA sequence for clone AA363226.

SEQ ID NO: 3528 represents the cDNA sequence for clone AA363268.

SEQ ID NO: 3529 represents the cDNA sequence for clone AA363270.

SEQ ID NO: 3530 represents the cDNA sequence for clone AA363271.

SEQ ID NO: 3531 represents the cDNA sequence for clone AA363307.

SEQ ID NO: 3532 represents the cDNA sequence for clone AA363308.

SEQ ID NO: 3533 represents the cDNA sequence for clone AA363312.

SEQ ID NO: 3534 represents the cDNA sequence for clone AA363315.

SEQ ID NO: 3535 represents the cDNA sequence for clone AA363324.

SEQ ID NO: 3536 represents the cDNA sequence for clone AA363327.

SEQ ID NO: 3537 represents the cDNA sequence for clone AA363331.

SEQ ID NO: 3538 represents the cDNA sequence for clone AA363334.

SEQ ID NO: 3539 represents the cDNA sequence for clone AA363342.

SEQ ID NO: 3540 represents the cDNA sequence for clone AA363345.

SEQ ID NO: 3541 represents the cDNA sequence for clone AA363113.

SEQ ID NO: 3542 represents the cDNA sequence for clone AA363206.

SEQ ID NO: 3543 represents the cDNA sequence for clone AA363207.

SEQ ID NO: 3544 represents the cDNA sequence for clone AA363209.

SEQ ID NO: 3545 represents the cDNA sequence for clone AA363210.

SEQ ID NO: 3546 represents the cDNA sequence for clone AA363214.

SEQ ID NO: 3547 represents the cDNA sequence for clone AA363347.

SEQ ID NO: 3548 represents the cDNA sequence for clone AA363348.

SEQ ID NO: 3549 represents the cDNA sequence for clone AA363349.

SEQ ID NO: 3550 represents the cDNA sequence for clone AA363351.

SEQ ID NO: 3551 represents the cDNA sequence for clone AA363353.

SEQ ID NO: 3552 represents the cDNA sequence for clone AA363354.

SEQ ID NO: 3553 represents the cDNA sequence for clone AA363357.

SEQ ID NO: 3554 represents the cDNA sequence for clone AA363380.

SEQ ID NO: 3555 represents the cDNA sequence for clone AA363382.

SEQ ID NO: 3556 represents the cDNA sequence for clone AA363389.

SEQ ID NO: 3557 represents the cDNA sequence for clone AA363393.

SEQ ID NO: 3558 represents the cDNA sequence for clone AA363396.

SEQ ID NO: 3559 represents the cDNA sequence for clone AA363400.

SEQ ID NO: 3560 represents the cDNA sequence for clone AA363401.

SEQ ID NO: 3561 represents the cDNA sequence for clone AA363402.

SEQ ID NO: 3562 represents the cDNA sequence for clone AA363403.

SEQ ID NO: 3563 represents the cDNA sequence for clone AA363405.

SEQ ID NO: 3564 represents the cDNA sequence for clone AA363406.

SEQ ID NO: 3565 represents the cDNA sequence for clone AA363407.

SEQ ID NO: 3566 represents the cDNA sequence for clone AA363410.

SEQ ID NO: 3567 represents the cDNA sequence for clone AA363411.

SEQ ID NO: 3568 represents the cDNA sequence for clone AA363413.

SEQ ID NO: 3569 represents the cDNA sequence for clone AA363416.

SEQ ID NO: 3570 represents the cDNA sequence for clone AA363417.

SEQ ID NO: 3571 represents the cDNA sequence for clone AA363365.

SEQ ID NO: 3572 represents the cDNA sequence for clone AA363366.

SEQ ID NO: 3573 represents the cDNA sequence for clone AA363370.

SEQ ID NO: 3574 represents the cDNA sequence for clone AA363373.

SEQ ID NO: 3575 represents the cDNA sequence for clone AA363376.

SEQ ID NO: 3576 represents the cDNA sequence for clone AA363377.

SEQ ID NO: 3577 represents the cDNA sequence for clone AA363290.

SEQ ID NO: 3578 represents the cDNA sequence for clone AA363291.

SEQ ID NO: 3579 represents the cDNA sequence for clone AA363292.

SEQ ID NO: 3580 represents the cDNA sequence for clone AA363296.

SEQ ID NO: 3581 represents the cDNA sequence for clone AA363430.

SEQ ID NO: 3582 represents the cDNA sequence for clone AA363432.

SEQ ID NO: 3583 represents the cDNA sequence for clone AA363446.

SEQ ID NO: 3584 represents the cDNA sequence for clone AA363447.

SEQ ID NO: 3585 represents the cDNA sequence for clone AA363448.

SEQ ID NO: 3586 represents the cDNA sequence for clone AA363472.

SEQ ID NO: 3587 represents the cDNA sequence for clone AA363476.

SEQ ID NO: 3588 represents the cDNA sequence for clone AA363477.

SEQ ID NO: 3589 represents the cDNA sequence for clone AA363478.

SEQ ID NO: 3590 represents the cDNA sequence for clone AA363487.

SEQ ID NO: 3591 represents the cDNA sequence for clone AA363488.

SEQ ID NO: 3592 represents the cDNA sequence for clone AA363491.

SEQ ID NO: 3593 represents the cDNA sequence for clone AA363498.

SEQ ID NO: 3594 represents the cDNA sequence for clone AA363509.

SEQ ID NO: 3595 represents the cDNA sequence for clone AA363277.

SEQ ID NO: 3596 represents the cDNA sequence for clone AA363280.

SEQ ID NO: 3597 represents the cDNA sequence for clone AA363281.

SEQ ID NO: 3598 represents the cDNA sequence for clone AA363285.

SEQ ID NO: 3599 represents the cDNA sequence for clone AA399124.

SEQ ID NO: 3600 represents the cDNA sequence for clone AA399134.

SEQ ID NO: 3601 represents the cDNA sequence for clone AA399136.

SEQ ID NO: 3602 represents the cDNA sequence for clone AA399139.

SEQ ID NO: 3603 represents the cDNA sequence for clone AA399141.

SEQ ID NO: 3604 represents the cDNA sequence for clone AA398948.

SEQ ID NO: 3605 represents the cDNA sequence for clone AA399613.

SEQ ID NO: 3606 represents the cDNA sequence for clone AA399615.

SEQ ID NO: 3607 represents the cDNA sequence for clone AA399144.

SEQ ID NO: 3608 represents the cDNA sequence for clone AA399147.

SEQ ID NO: 3609 represents the cDNA sequence for clone AA399149.

SEQ ID NO: 3610 represents the cDNA sequence for clone AA399170.

SEQ ID NO: 3611 represents the cDNA sequence for clone AA399172.

SEQ ID NO: 3612 represents the cDNA sequence for clone AA399175.

SEQ ID NO: 3613 represents the cDNA sequence for clone AA399176.

SEQ ID NO: 3614 represents the cDNA sequence for clone AA399177.

SEQ ID NO: 3615 represents the cDNA sequence for clone AA399178.

SEQ ID NO: 3616 represents the cDNA sequence for clone AA399179.

SEQ ID NO: 3617 represents the cDNA sequence for clone AA399181.

SEQ ID NO: 3618 represents the cDNA sequence for clone AA399191.

SEQ ID NO: 3619 represents the cDNA sequence for clone AA399194.

SEQ ID NO: 3620 represents the cDNA sequence for clone AA399197.

SEQ ID NO: 3621 represents the cDNA sequence for clone AA399199.

SEQ ID NO: 3622 represents the cDNA sequence for clone AA399203.

SEQ ID NO: 3623 represents the cDNA sequence for clone AA399087.

SEQ ID NO: 3624 represents the cDNA sequence for clone AA399162.

SEQ ID NO: 3625 represents the cDNA sequence for clone AA399331.

SEQ ID NO: 3626 represents the cDNA sequence for clone AA399334.

SEQ ID NO: 3627 represents the cDNA sequence for clone AA399335.

SEQ ID NO: 3628 represents the cDNA sequence for clone AA399337.

SEQ ID NO: 3629 represents the cDNA sequence for clone AA399338.

SEQ ID NO: 3630 represents the cDNA sequence for clone AA399342.

SEQ ID NO: 3631 represents the cDNA sequence for clone AA399153.

SEQ ID NO: 3632 represents the cDNA sequence for clone AA399155.

SEQ ID NO: 3633 represents the cDNA sequence for clone AA399158.

SEQ ID NO: 3634 represents the cDNA sequence for clone AA399305.

SEQ ID NO: 3635 represents the cDNA sequence for clone AA399355.

SEQ ID NO: 3636 represents the cDNA sequence for clone AA399221.

SEQ ID NO: 3637 represents the cDNA sequence for clone AA399364.

SEQ ID NO: 3638 represents the cDNA sequence for clone AA399416.

SEQ ID NO: 3639 represents the cDNA sequence for clone AA399417.

SEQ ID NO: 3640 represents the cDNA sequence for clone AA399438.

SEQ ID NO: 3641 represents the cDNA sequence for clone AA399441.

SEQ ID NO: 3642 represents the cDNA sequence for clone AA399450.

SEQ ID NO: 3643 represents the cDNA sequence for clone AA399453.

SEQ ID NO: 3644 represents the cDNA sequence for clone AA399420.

SEQ ID NO: 3645 represents the cDNA sequence for clone AA399422.

SEQ ID NO: 3646 represents the cDNA sequence for clone AA399462.

SEQ ID NO: 3647 represents the cDNA sequence for clone AA399467.

SEQ ID NO: 3648 represents the cDNA sequence for clone AA399468.

SEQ ID NO: 3649 represents the cDNA sequence for clone AA399472.

SEQ ID NO: 3650 represents the cDNA sequence for clone AA399478.

SEQ ID NO: 3651 represents the cDNA sequence for clone AA399293.

SEQ ID NO: 3652 represents the cDNA sequence for clone AA399296.

SEQ ID NO: 3653 represents the cDNA sequence for clone AA399455.

SEQ ID NO: 3654 represents the cDNA sequence for clone AA401332.

SEQ ID NO: 3655 represents the cDNA sequence for clone AA401359.

SEQ ID NO: 3656 represents the cDNA sequence for clone AA401446.

SEQ ID NO: 3657 represents the cDNA sequence for clone AA401452.

SEQ ID NO: 3658 represents the cDNA sequence for clone AA401459.

SEQ ID NO: 3659 represents the cDNA sequence for clone AA400885.

SEQ ID NO: 3660 represents the cDNA sequence for clone AA400888.

SEQ ID NO: 3661 represents the cDNA sequence for clone AA400891.

SEQ ID NO: 3662 represents the cDNA sequence for clone AA400938.

SEQ ID NO: 3663 represents the cDNA sequence for clone AA400941.

SEQ ID NO: 3664 represents the cDNA sequence for clone AA400943.

SEQ ID NO: 3665 represents the cDNA sequence for clone AA400945.

SEQ ID NO: 3666 represents the cDNA sequence for clone AA400967.

SEQ ID NO: 3667 represents the cDNA sequence for clone AA400951.

SEQ ID NO: 3668 represents the cDNA sequence for clone AA400983.

SEQ ID NO: 3669 represents the cDNA sequence for clone AA400997.

SEQ ID NO: 3670 represents the cDNA sequence for clone AA400960.

SEQ ID NO: 3671 represents the cDNA sequence for clone AA400961.

SEQ ID NO: 3672 represents the cDNA sequence for clone AA400962.

SEQ ID NO: 3673 represents the cDNA sequence for clone AA400877.

SEQ ID NO: 3674 represents the cDNA sequence for clone AA400879.

SEQ ID NO: 3675 represents the cDNA sequence for clone AA400883.

SEQ ID NO: 3676 represents the cDNA sequence for clone AA400884.

SEQ ID NO: 3677 represents the cDNA sequence for clone AA401030.

SEQ ID NO: 3678 represents the cDNA sequence for clone AA401031.

SEQ ID NO: 3679 represents the cDNA sequence for clone AA401037.

SEQ ID NO: 3680 represents the cDNA sequence for clone AA401046.

SEQ ID NO: 3681 represents the cDNA sequence for clone AA401049.

SEQ ID NO: 3682 represents the cDNA sequence for clone AA401051.

SEQ ID NO: 3683 represents the cDNA sequence for clone AA401053.

SEQ ID NO: 3684 represents the cDNA sequence for clone AA401022.

SEQ ID NO: 3685 represents the cDNA sequence for clone AA401067.

SEQ ID NO: 3686 represents the cDNA sequence for clone AA401068.

SEQ ID NO: 3687 represents the cDNA sequence for clone AA400979.

SEQ ID NO: 3688 represents the cDNA sequence for clone AA400813.

SEQ ID NO: 3689 represents the cDNA sequence for clone AA400815.

SEQ ID NO: 3690 represents the cDNA sequence for clone AA401072.

SEQ ID NO: 3691 represents the cDNA sequence for clone AA401073.

SEQ ID NO: 3692 represents the cDNA sequence for clone AA401101.

SEQ ID NO: 3693 represents the cDNA sequence for clone AA401114.

SEQ ID NO: 3694 represents the cDNA sequence for clone AA401115.

SEQ ID NO: 3695 represents the cDNA sequence for clone AA401124.

SEQ ID NO: 3696 represents the cDNA sequence for clone AA401125.

SEQ ID NO: 3697 represents the cDNA sequence for clone AA401129.

SEQ ID NO: 3698 represents the cDNA sequence for clone AA401132.

SEQ ID NO: 3699 represents the cDNA sequence for clone AA401028.

SEQ ID NO: 3700 represents the cDNA sequence for clone AA401055.

SEQ ID NO: 3701 represents the cDNA sequence for clone AA401060.

SEQ ID NO: 3702 represents the cDNA sequence for clone AA401134.

SEQ ID NO: 3703 represents the cDNA sequence for clone AA401156.

SEQ ID NO: 3704 represents the cDNA sequence for clone AA401158.

SEQ ID NO: 3705 represents the cDNA sequence for clone AA401164.

SEQ ID NO: 3706 represents the cDNA sequence for clone AA401166.

SEQ ID NO: 3707 represents the cDNA sequence for clone AA401167.

SEQ ID NO: 3708 represents the cDNA sequence for clone AA401177.

SEQ ID NO: 3709 represents the cDNA sequence for clone AA401182.

SEQ ID NO: 3710 represents the cDNA sequence for clone AA401183.

SEQ ID NO: 3711 represents the cDNA sequence for clone AA401184.

SEQ ID NO: 3712 represents the cDNA sequence for clone AA401185.

SEQ ID NO: 3713 represents the cDNA sequence for clone AA401192.

SEQ ID NO: 3714 represents the cDNA sequence for clone AA401149.

SEQ ID NO: 3715 represents the cDNA sequence for clone AA401093.

SEQ ID NO: 3716 represents the cDNA sequence for clone AA401196.

SEQ ID NO: 3717 represents the cDNA sequence for clone AA401212.

SEQ ID NO: 3718 represents the cDNA sequence for clone AA403288.

SEQ ID NO: 3719 represents the cDNA sequence for clone AA403293.

SEQ ID NO: 3720 represents the cDNA sequence for clone AA403297.

SEQ ID NO: 3721 represents the cDNA sequence for clone AA403298.

SEQ ID NO: 3722 represents the cDNA sequence for clone AA403301.

SEQ ID NO: 3723 represents the cDNA sequence for clone AA403310.

SEQ ID NO: 3724 represents the cDNA sequence for clone AA403314.

SEQ ID NO: 3725 represents the cDNA sequence for clone AA403268.

SEQ ID NO: 3726 represents the cDNA sequence for clone AA403272.

SEQ ID NO: 3727 represents the cDNA sequence for clone AA401887.

SEQ ID NO: 3728 represents the cDNA sequence for clone AA401888.

SEQ ID NO: 3729 represents the cDNA sequence for clone AA401890.

SEQ ID NO: 3730 represents the cDNA sequence for clone AA401826.

SEQ ID NO: 3731 represents the cDNA sequence for clone AA401910.

SEQ ID NO: 3732 represents the cDNA sequence for clone AA401912.

SEQ ID NO: 3733 represents the cDNA sequence for clone AA401913.

SEQ ID NO: 3734 represents the cDNA sequence for clone AA401917.

SEQ ID NO: 3735 represents the cDNA sequence for clone AA401918.

SEQ ID NO: 3736 represents the cDNA sequence for clone AA401947.

SEQ ID NO: 3737 represents the cDNA sequence for clone AA401949.

SEQ ID NO: 3738 represents the cDNA sequence for clone AA401950.

SEQ ID NO: 3739 represents the cDNA sequence for clone AA401952.

SEQ ID NO: 3740 represents the cDNA sequence for clone AA401963.

SEQ ID NO: 3741 represents the cDNA sequence for clone AA401968.

SEQ ID NO: 3742 represents the cDNA sequence for clone AA401923.

SEQ ID NO: 3743 represents the cDNA sequence for clone AA401925.

SEQ ID NO: 3744 represents the cDNA sequence for clone AA401927.

SEQ ID NO: 3745 represents the cDNA sequence for clone AA401931.

SEQ ID NO: 3746 represents the cDNA sequence for clone AA401933.

SEQ ID NO: 3747 represents the cDNA sequence for clone AA401934.

SEQ ID NO: 3748 represents the cDNA sequence for clone AA401935.

SEQ ID NO: 3749 represents the cDNA sequence for clone AA401936.

SEQ ID NO: 3750 represents the cDNA sequence for clone AA401989.

SEQ ID NO: 3751 represents the cDNA sequence for clone AA401991.

SEQ ID NO: 3752 represents the cDNA sequence for clone AA401994.

SEQ ID NO: 3753 represents the cDNA sequence for clone AA402013.

SEQ ID NO: 3754 represents the cDNA sequence for clone AA402028.

SEQ ID NO: 3755 represents the cDNA sequence for clone AA401995.

SEQ ID NO: 3756 represents the cDNA sequence for clone AA402000.

SEQ ID NO: 3757 represents the cDNA sequence for clone AA401969.

SEQ ID NO: 3758 represents the cDNA sequence for clone AA401971.

SEQ ID NO: 3759 represents the cDNA sequence for clone AA401975.

SEQ ID NO: 3760 represents the cDNA sequence for clone AA401976.

SEQ ID NO: 3761 represents the cDNA sequence for clone AA402039.

SEQ ID NO: 3762 represents the cDNA sequence for clone AA402043.

SEQ ID NO: 3763 represents the cDNA sequence for clone AA402046.

SEQ ID NO: 3764 represents the cDNA sequence for clone AA402047.

SEQ ID NO: 3765 represents the cDNA sequence for clone AA402070.

SEQ ID NO: 3766 represents the cDNA sequence for clone AA402074.

SEQ ID NO: 3767 represents the cDNA sequence for clone AA402076.

SEQ ID NO: 3768 represents the cDNA sequence for clone AA402077.

SEQ ID NO: 3769 represents the cDNA sequence for clone AA402078.

SEQ ID NO: 3770 represents the cDNA sequence for clone AA402081.

SEQ ID NO: 3771 represents the cDNA sequence for clone AA402082.

SEQ ID NO: 3772 represents the cDNA sequence for clone AA402085.

SEQ ID NO: 3773 represents the cDNA sequence for clone AA402091.

SEQ ID NO: 3774 represents the cDNA sequence for clone AA402092.

SEQ ID NO: 3775 represents the cDNA sequence for clone AA402094.

SEQ ID NO: 3776 represents the cDNA sequence for clone AA402099.

SEQ ID NO: 3777 represents the cDNA sequence for clone AA402104.

SEQ ID NO: 3778 represents the cDNA sequence for clone AA402106.

SEQ ID NO: 3779 represents the cDNA sequence for clone AA402107.

SEQ ID NO: 3780 represents the cDNA sequence for clone AA402132.

SEQ ID NO: 3781 represents the cDNA sequence for clone AA402111.

SEQ ID NO: 3782 represents the cDNA sequence for clone AA402113.

SEQ ID NO: 3783 represents the cDNA sequence for clone AA402116.

SEQ ID NO: 3784 represents the cDNA sequence for clone AA402149.

SEQ ID NO: 3785 represents the cDNA sequence for clone AA402150.

SEQ ID NO: 3786 represents the cDNA sequence for clone AA402158.

SEQ ID NO: 3787 represents the cDNA sequence for clone AA402160.

SEQ ID NO: 3788 represents the cDNA sequence for clone AA402164.

SEQ ID NO: 3789 represents the cDNA sequence for clone AA402169.

SEQ ID NO: 3790 represents the cDNA sequence for clone AA402192.

SEQ ID NO: 3791 represents the cDNA sequence for clone AA402196.

SEQ ID NO: 3792 represents the cDNA sequence for clone AA402219.

SEQ ID NO: 3793 represents the cDNA sequence for clone AA402200.

SEQ ID NO: 3794 represents the cDNA sequence for clone AA402238.

SEQ ID NO: 3795 represents the cDNA sequence for clone AA402253.

SEQ ID NO: 3796 represents the cDNA sequence for clone AA402257.

SEQ ID NO: 3797 represents the cDNA sequence for clone AA402207.

SEQ ID NO: 3798 represents the cDNA sequence for clone AA402212.

SEQ ID NO: 3799 represents the cDNA sequence for clone AA402214.

SEQ ID NO: 3800 represents the cDNA sequence for clone AA402260.

SEQ ID NO: 3801 represents the cDNA sequence for clone AA402266.

SEQ ID NO: 3802 represents the cDNA sequence for clone AA402288.

SEQ ID NO: 3803 represents the cDNA sequence for clone AA402289.

SEQ ID NO: 3804 represents the cDNA sequence for clone AA402294.

SEQ ID NO: 3805 represents the cDNA sequence for clone AA402295.

SEQ ID NO: 3806 represents the cDNA sequence for clone AA402296.

SEQ ID NO: 3807 represents the cDNA sequence for clone AA402320.

SEQ ID NO: 3808 represents the cDNA sequence for clone AA402323.

SEQ ID NO: 3809 represents the cDNA sequence for clone AA402312.

SEQ ID NO: 3810 represents the cDNA sequence for clone AA402275.

SEQ ID NO: 3811 represents the cDNA sequence for clone AA402279.

SEQ ID NO: 3812 represents the cDNA sequence for clone AA402343.

SEQ ID NO: 3813 represents the cDNA sequence for clone AA402348.

SEQ ID NO: 3814 represents the cDNA sequence for clone AA402350.

SEQ ID NO: 3815 represents the cDNA sequence for clone AA402356.

SEQ ID NO: 3816 represents the cDNA sequence for clone AA402381.

SEQ ID NO: 3817 represents the cDNA sequence for clone AA402387.

SEQ ID NO: 3818 represents the cDNA sequence for clone AA402302.

SEQ ID NO: 3819 represents the cDNA sequence for clone AA402305.

SEQ ID NO: 3820 represents the cDNA sequence for clone AA402370.

SEQ ID NO: 3821 represents the cDNA sequence for clone AA402395.

SEQ ID NO: 3822 represents the cDNA sequence for clone AA402496.

SEQ ID NO: 3823 represents the cDNA sequence for clone AA402498.

SEQ ID NO: 3824 represents the cDNA sequence for clone AA402502.

SEQ ID NO: 3825 represents the cDNA sequence for clone AA402525.

SEQ ID NO: 3826 represents the cDNA sequence for clone AA402531.

SEQ ID NO: 3827 represents the cDNA sequence for clone AA402546.

SEQ ID NO: 3828 represents the cDNA sequence for clone AA402565.

SEQ ID NO: 3829 represents the cDNA sequence for clone AA402571.

SEQ ID NO: 3830 represents the cDNA sequence for clone AA402572.

SEQ ID NO: 3831 represents the cDNA sequence for clone AA402581.

SEQ ID NO: 3832 represents the cDNA sequence for clone AA402586.

SEQ ID NO: 3833 represents the cDNA sequence for clone AA402590.

SEQ ID NO: 3834 represents the cDNA sequence for clone AA402591.

SEQ ID NO: 3835 represents the cDNA sequence for clone AA402592.

SEQ ID NO: 3836 represents the cDNA sequence for clone AA402558.

SEQ ID NO: 3837 represents the cDNA sequence for clone AA402547.

SEQ ID NO: 3838 represents the cDNA sequence for clone AA402548.

SEQ ID NO: 3839 represents the cDNA sequence for clone AA402606.

SEQ ID NO: 3840 represents the cDNA sequence for clone AA402613.

SEQ ID NO: 3841 represents the cDNA sequence for clone AA402633.

SEQ ID NO: 3842 represents the cDNA sequence for clone AA402637.

SEQ ID NO: 3843 represents the cDNA sequence for clone AA402638.

SEQ ID NO: 3844 represents the cDNA sequence for clone AA402659.

SEQ ID NO: 3845 represents the cDNA sequence for clone AA402618.

SEQ ID NO: 3846 represents the cDNA sequence for clone AA402620.

SEQ ID NO: 3847 represents the cDNA sequence for clone AA402621.

SEQ ID NO: 3848 represents the cDNA sequence for clone AA402667.

SEQ ID NO: 3849 represents the cDNA sequence for clone AA402668.

SEQ ID NO: 3850 represents the cDNA sequence for clone AA402670.

SEQ ID NO: 3851 represents the cDNA sequence for clone AA402673.

SEQ ID NO: 3852 represents the cDNA sequence for clone AA402649.

SEQ ID NO: 3853 represents the cDNA sequence for clone AA402690.

SEQ ID NO: 3854 represents the cDNA sequence for clone AA402713.

SEQ ID NO: 3855 represents the cDNA sequence for clone AA402719.

SEQ ID NO: 3856 represents the cDNA sequence for clone AA402723.

SEQ ID NO: 3857 represents the cDNA sequence for clone AA402623.

SEQ ID NO: 3858 represents the cDNA sequence for clone AA402627.

SEQ ID NO: 3859 represents the cDNA sequence for clone AA402707.

SEQ ID NO: 3860 represents the cDNA sequence for clone AA402749.

SEQ ID NO: 3861 represents the cDNA sequence for clone AA402753.

SEQ ID NO: 3862 represents the cDNA sequence for clone AA402777.

SEQ ID NO: 3863 represents the cDNA sequence for clone AA402782.

SEQ ID NO: 3864 represents the cDNA sequence for clone AA402681.

SEQ ID NO: 3865 represents the cDNA sequence for clone AA402767.

SEQ ID NO: 3866 represents the cDNA sequence for clone AA402801.

SEQ ID NO: 3867 represents the cDNA sequence for clone AA402803.

SEQ ID NO: 3868 represents the cDNA sequence for clone AA402807.

SEQ ID NO: 3869 represents the cDNA sequence for clone AA402829.

SEQ ID NO: 3870 represents the cDNA sequence for clone AA402831.

SEQ ID NO: 3871 represents the cDNA sequence for clone AA402838.

SEQ ID NO: 3872 represents the cDNA sequence for clone AA402840.

SEQ ID NO: 3873 represents the cDNA sequence for clone AA402843.

SEQ ID NO: 3874 represents the cDNA sequence for clone AA402844.

SEQ ID NO: 3875 represents the cDNA sequence for clone AA402845.

SEQ ID NO: 3876 represents the cDNA sequence for clone AA402848.

SEQ ID NO: 3877 represents the cDNA sequence for clone AA402883.

SEQ ID NO: 3878 represents the cDNA sequence for clone AA402910.

SEQ ID NO: 3879 represents the cDNA sequence for clone AA402921.

SEQ ID NO: 3880 represents the cDNA sequence for clone AA402932.

SEQ ID NO: 3881 represents the cDNA sequence for clone AA402934.

SEQ ID NO: 3882 represents the cDNA sequence for clone AA402935.

SEQ ID NO: 3883 represents the cDNA sequence for clone AA402809.

SEQ ID NO: 3884 represents the cDNA sequence for clone AA402814.

SEQ ID NO: 3885 represents the cDNA sequence for clone AA402895.

SEQ ID NO: 3886 represents the cDNA sequence for clone AA402937.

SEQ ID NO: 3887 represents the cDNA sequence for clone AA402939.

SEQ ID NO: 3888 represents the cDNA sequence for clone AA402951.

SEQ ID NO: 3889 represents the cDNA sequence for clone AA402970.

SEQ ID NO: 3890 represents the cDNA sequence for clone AA402971.

SEQ ID NO: 3891 represents the cDNA sequence for clone AA402976.

SEQ ID NO: 3892 represents the cDNA sequence for clone AA402987.

SEQ ID NO: 3893 represents the cDNA sequence for clone AA402993.

SEQ ID NO: 3894 represents the cDNA sequence for clone AA402821.

SEQ ID NO: 3895 represents the cDNA sequence for clone AA402823.

SEQ ID NO: 3896 represents the cDNA sequence for clone AA402959.

SEQ ID NO: 3897 represents the cDNA sequence for clone AA403004.

SEQ ID NO: 3898 represents the cDNA sequence for clone AA403010.

SEQ ID NO: 3899 represents the cDNA sequence for clone AA403029.

SEQ ID NO: 3900 represents the cDNA sequence for clone AA401711.

SEQ ID NO: 3901 represents the cDNA sequence for clone AA401714.

SEQ ID NO: 3902 represents the cDNA sequence for clone AA401752.

SEQ ID NO: 3903 represents the cDNA sequence for clone AA401754.

SEQ ID NO: 3904 represents the cDNA sequence for clone AA401756.

SEQ ID NO: 3905 represents the cDNA sequence for clone AA401757.

SEQ ID NO: 3906 represents the cDNA sequence for clone AA401761.

SEQ ID NO: 3907 represents the cDNA sequence for clone AA404576.

SEQ ID NO: 3908 represents the cDNA sequence for clone AA404577.

SEQ ID NO: 3909 represents the cDNA sequence for clone AA404581.

SEQ ID NO: 3910 represents the cDNA sequence for clone AA404583.

SEQ ID NO: 3911 represents the cDNA sequence for clone AA404584.

SEQ ID NO: 3912 represents the cDNA sequence for clone AA404589.

SEQ ID NO: 3913 represents the cDNA sequence for clone AA404593.

SEQ ID NO: 3914 represents the cDNA sequence for clone AA404595.

SEQ ID NO: 3915 represents the cDNA sequence for clone AA404598.

SEQ ID NO: 3916 represents the cDNA sequence for clone AA404600.

SEQ ID NO: 3917 represents the cDNA sequence for clone AA404601.

SEQ ID NO: 3918 represents the cDNA sequence for clone AA404609.

SEQ ID NO: 3919 represents the cDNA sequence for clone AA404613.

SEQ ID NO: 3920 represents the cDNA sequence for clone AA404635.

SEQ ID NO: 3921 represents the cDNA sequence for clone AA404636.

SEQ ID NO: 3922 represents the cDNA sequence for clone AA404637.

SEQ ID NO: 3923 represents the cDNA sequence for clone AA404653.

SEQ ID NO: 3924 represents the cDNA sequence for clone AA404617.

SEQ ID NO: 3925 represents the cDNA sequence for clone AA404618.

SEQ ID NO: 3926 represents the cDNA sequence for clone AA404684.

SEQ ID NO: 3927 represents the cDNA sequence for clone AA404686.

SEQ ID NO: 3928 represents the cDNA sequence for clone AA404695.

SEQ ID NO: 3929 represents the cDNA sequence for clone AA404630.

SEQ ID NO: 3930 represents the cDNA sequence for clone AA404631.

SEQ ID NO: 3931 represents the cDNA sequence for clone AA404707.

SEQ ID NO: 3932 represents the cDNA sequence for clone AA404710.

SEQ ID NO: 3933 represents the cDNA sequence for clone AA404715.

SEQ ID NO: 3934 represents the cDNA sequence for clone AA404716.

SEQ ID NO: 3935 represents the cDNA sequence for clone AA404718.

SEQ ID NO: 3936 represents the cDNA sequence for clone AA404719.

SEQ ID NO: 3937 represents the cDNA sequence for clone AA404723.

SEQ ID NO: 3938 represents the cDNA sequence for clone AA404736.

SEQ ID NO: 3939 represents the cDNA sequence for clone AA404644.

SEQ ID NO: 3940 represents the cDNA sequence for clone AA404648.

SEQ ID NO: 3941 represents the cDNA sequence for clone AA404649.

SEQ ID NO: 3942 represents the cDNA sequence for clone AA404726.

SEQ ID NO: 3943 represents the cDNA sequence for clone AA404731.

SEQ ID NO: 3944 represents the cDNA sequence for clone AA404733.

SEQ ID NO: 3945 represents the cDNA sequence for clone AA404215.

SEQ ID NO: 3946 represents the cDNA sequence for clone AA404220.

SEQ ID NO: 3947 represents the cDNA sequence for clone AA404223.

SEQ ID NO: 3948 represents the cDNA sequence for clone AA404225.

SEQ ID NO: 3949 represents the cDNA sequence for clone AA404299.

SEQ ID NO: 3950 represents the cDNA sequence for clone AA404257.

SEQ ID NO: 3951 represents the cDNA sequence for clone AA404311.

SEQ ID NO: 3952 represents the cDNA sequence for clone AA404314.

SEQ ID NO: 3953 represents the cDNA sequence for clone AA404315.

SEQ ID NO: 3954 represents the cDNA sequence for clone AA405266.

SEQ ID NO: 3955 represents the cDNA sequence for clone AA405280.

SEQ ID NO: 3956 represents the cDNA sequence for clone AA405281.

SEQ ID NO: 3957 represents the cDNA sequence for clone AA405248.

SEQ ID NO: 3958 represents the cDNA sequence for clone AA405250.

SEQ ID NO: 3959 represents the cDNA sequence for clone AA405302.

SEQ ID NO: 3960 represents the cDNA sequence for clone AA405317.

SEQ ID NO: 3961 represents the cDNA sequence for clone AA405288.

SEQ ID NO: 3962 represents the cDNA sequence for clone AA405167.

SEQ ID NO: 3963 represents the cDNA sequence for clone AA405678.

SEQ ID NO: 3964 represents the cDNA sequence for clone AA405679.

SEQ ID NO: 3965 represents the cDNA sequence for clone AA405680.

SEQ ID NO: 3966 represents the cDNA sequence for clone AA405168.

SEQ ID NO: 3967 represents the cDNA sequence for clone AA405169.

SEQ ID NO: 3968 represents the cDNA sequence for clone AA405171.

SEQ ID NO: 3969 represents the cDNA sequence for clone AA405198.

SEQ ID NO: 3970 represents the cDNA sequence for clone AA405204.

SEQ ID NO: 3971 represents the cDNA sequence for clone AA405206.

SEQ ID NO: 3972 represents the cDNA sequence for clone AA405395.

SEQ ID NO: 3973 represents the cDNA sequence for clone AA405401.

SEQ ID NO: 3974 represents the cDNA sequence for clone AA405402.

SEQ ID NO: 3975 represents the cDNA sequence for clone AA405403.

SEQ ID NO: 3976 represents the cDNA sequence for clone AA405404.

SEQ ID NO: 3977 represents the cDNA sequence for clone AA405405.

SEQ ID NO: 3978 represents the cDNA sequence for clone AA405407.

SEQ ID NO: 3979 represents the cDNA sequence for clone AA405423.

SEQ ID NO: 3980 represents the cDNA sequence for clone AA405424.

SEQ ID NO: 3981 represents the cDNA sequence for clone AA405746.

SEQ ID NO: 3982 represents the cDNA sequence for clone AA405747.

SEQ ID NO: 3983 represents the cDNA sequence for clone AA405190.

SEQ ID NO: 3984 represents the cDNA sequence for clone AA405192.

SEQ ID NO: 3985 represents the cDNA sequence for clone AA405727.

SEQ ID NO: 3986 represents the cDNA sequence for clone AA405763.

SEQ ID NO: 3987 represents the cDNA sequence for clone AA405793.

SEQ ID NO: 3988 represents the cDNA sequence for clone AA405798.

SEQ ID NO: 3989 represents the cDNA sequence for clone AA405804.

SEQ ID NO: 3990 represents the cDNA sequence for clone AA405805.

SEQ ID NO: 3991 represents the cDNA sequence for clone AA405869.

SEQ ID NO: 3992 represents the cDNA sequence for clone AA405871.

SEQ ID NO: 3993 represents the cDNA sequence for clone AA405872.

SEQ ID NO: 3994 represents the cDNA sequence for clone AA405881.

SEQ ID NO: 3995 represents the cDNA sequence for clone AA405883.

SEQ ID NO: 3996 represents the cDNA sequence for clone AA405886.

SEQ ID NO: 3997 represents the cDNA sequence for clone AA405887.

SEQ ID NO: 3998 represents the cDNA sequence for clone AA405888.

SEQ ID NO: 3999 represents the cDNA sequence for clone AA405845.

SEQ ID NO: 4000 represents the cDNA sequence for clone AA405847.

SEQ ID NO: 4001 represents the cDNA sequence for clone AA405894.

SEQ ID NO: 4002 represents the cDNA sequence for clone AA405899.

SEQ ID NO: 4003 represents the cDNA sequence for clone AA405919.

SEQ ID NO: 4004 represents the cDNA sequence for clone AA405921.

SEQ ID NO: 4005 represents the cDNA sequence for clone AA405922.

SEQ ID NO: 4006 represents the cDNA sequence for clone AA405924.

SEQ ID NO: 4007 represents the cDNA sequence for clone AA405928.

SEQ ID NO: 4008 represents the cDNA sequence for clone AA406000.

SEQ ID NO: 4009 represents the cDNA sequence for clone AA406002.

SEQ ID NO: 4010 represents the cDNA sequence for clone AA406003.

SEQ ID NO: 4011 represents the cDNA sequence for clone AA406006.

SEQ ID NO: 4012 represents the cDNA sequence for clone AA406008.

SEQ ID NO: 4013 represents the cDNA sequence for clone AA406016.

SEQ ID NO: 4014 represents the cDNA sequence for clone AA406017.

SEQ ID NO: 4015 represents the cDNA sequence for clone AA405975.

SEQ ID NO: 4016 represents the cDNA sequence for clone AA405976.

SEQ ID NO: 4017 represents the cDNA sequence for clone AA405977.

SEQ ID NO: 4018 represents the cDNA sequence for clone AA405978.

SEQ ID NO: 4019 represents the cDNA sequence for clone AA406021.

SEQ ID NO: 4020 represents the cDNA sequence for clone AA406027.

SEQ ID NO: 4021 represents the cDNA sequence for clone AA406028.

SEQ ID NO: 4022 represents the cDNA sequence for clone AA406031.

SEQ ID NO: 4023 represents the cDNA sequence for clone AA401244.

SEQ ID NO: 4024 represents the cDNA sequence for clone AA411249.

SEQ ID NO: 4025 represents the cDNA sequence for clone AA411260.

SEQ ID NO: 4026 represents the cDNA sequence for clone AA411613.

SEQ ID NO: 4027 represents the cDNA sequence for clone AA411272.

SEQ ID NO: 4028 represents the cDNA sequence for clone AA411315.

SEQ ID NO: 4029 represents the cDNA sequence for clone AA411316.

SEQ ID NO: 4030 represents the cDNA sequence for clone AA411322.

SEQ ID NO: 4031 represents the cDNA sequence for clone AA411323.

SEQ ID NO: 4032 represents the cDNA sequence for clone AA411211.

SEQ ID NO: 4033 represents the cDNA sequence for clone AA411214.

SEQ ID NO: 4034 represents the cDNA sequence for clone AA411372.

SEQ ID NO: 4035 represents the cDNA sequence for clone AA411351.

SEQ ID NO: 4036 represents the cDNA sequence for clone AA411355.

SEQ ID NO: 4037 represents the cDNA sequence for clone AA411403.

SEQ ID NO: 4038 represents the cDNA sequence for clone AA411428.

SEQ ID NO: 4039 represents the cDNA sequence for clone AA411430.

SEQ ID NO: 4040 represents the cDNA sequence for clone AA411432.

SEQ ID NO: 4041 represents the cDNA sequence for clone AA411437.

SEQ ID NO: 4042 represents the cDNA sequence for clone AA411441.

SEQ ID NO: 4043 represents the cDNA sequence for clone AA411443.

SEQ ID NO: 4044 represents the cDNA sequence for clone AA411449.

SEQ ID NO: 4045 represents the cDNA sequence for clone AA411453.

SEQ ID NO: 4046 represents the cDNA sequence for clone AA411458.

SEQ ID NO: 4047 represents the cDNA sequence for clone AA411460.

SEQ ID NO: 4048 represents the cDNA sequence for clone AA411469.

SEQ ID NO: 4049 represents the cDNA sequence for clone AA411304.

SEQ ID NO: 4050 represents the cDNA sequence for clone AA411418.

SEQ ID NO: 4051 represents the cDNA sequence for clone AA411423.

SEQ ID NO: 4052 represents the cDNA sequence for clone AA411424.

SEQ ID NO: 4053 represents the cDNA sequence for clone AA411473.

SEQ ID NO: 4054 represents the cDNA sequence for clone AA411474.

SEQ ID NO: 4055 represents the cDNA sequence for clone AA411504.

SEQ ID NO: 4056 represents the cDNA sequence for clone AA411506.

SEQ ID NO: 4057 represents the cDNA sequence for clone AA411515.

SEQ ID NO: 4058 represents the cDNA sequence for clone AA411516.

SEQ ID NO: 4059 represents the cDNA sequence for clone AA411521.

SEQ ID NO: 4060 represents the cDNA sequence for clone AA411523.

SEQ ID NO: 4061 represents the cDNA sequence for clone AA411537.

SEQ ID NO: 4062 represents the cDNA sequence for clone AA411538.

SEQ ID NO: 4063 represents the cDNA sequence for clone AA411550.

SEQ ID NO: 4064 represents the cDNA sequence for clone AA411551.

SEQ ID NO: 4065 represents the cDNA sequence for clone AA411573.

SEQ ID NO: 4066 represents the cDNA sequence for clone AA411580.

SEQ ID NO: 4067 represents the cDNA sequence for clone AA411586.

SEQ ID NO: 4068 represents the cDNA sequence for clone AA411587.

SEQ ID NO: 4069 represents the cDNA sequence for clone AA411590.

SEQ ID NO: 4070 represents the cDNA sequence for clone AA411597.

SEQ ID NO: 4071 represents the cDNA sequence for clone AA410233.

SEQ ID NO: 4072 represents the cDNA sequence for clone AA410236.

SEQ ID NO: 4073 represents the cDNA sequence for clone AA410240.

SEQ ID NO: 4074 represents the cDNA sequence for clone AA411492.

SEQ ID NO: 4075 represents the cDNA sequence for clone AA410245.

SEQ ID NO: 4076 represents the cDNA sequence for clone AA410247.

SEQ ID NO: 4077 represents the cDNA sequence for clone AA410250.

SEQ ID NO: 4078 represents the cDNA sequence for clone AA410253.

SEQ ID NO: 4079 represents the cDNA sequence for clone AA410276.

SEQ ID NO: 4080 represents the cDNA sequence for clone AA410595.

SEQ ID NO: 4081 represents the cDNA sequence for clone AA410596.

SEQ ID NO: 4082 represents the cDNA sequence for clone AA410601.

SEQ ID NO: 4083 represents the cDNA sequence for clone AA410412.

SEQ ID NO: 4084 represents the cDNA sequence for clone AA410413.

SEQ ID NO: 4085 represents the cDNA sequence for clone AA410557.

SEQ ID NO: 4086 represents the cDNA sequence for clone AA410558.

SEQ ID NO: 4087 represents the cDNA sequence for clone AA410631.

SEQ ID NO: 4088 represents the cDNA sequence for clone AA410639.

SEQ ID NO: 4089 represents the cDNA sequence for clone AA410645.

SEQ ID NO: 4090 represents the cDNA sequence for clone AA410648.

SEQ ID NO: 4091 represents the cDNA sequence for clone AA410656.

SEQ ID NO: 4092 represents the cDNA sequence for clone AA410664.

SEQ ID NO: 4093 represents the cDNA sequence for clone AA410615.

SEQ ID NO: 4094 represents the cDNA sequence for clone AA410617.

SEQ ID NO: 4095 represents the cDNA sequence for clone AA410618.

SEQ ID NO: 4096 represents the cDNA sequence for clone AA410620.

SEQ ID NO: 4097 represents the cDNA sequence for clone AA410621.

SEQ ID NO: 4098 represents the cDNA sequence for clone AA410475.

SEQ ID NO: 4099 represents the cDNA sequence for clone AA410476.

SEQ ID NO: 4100 represents the cDNA sequence for clone AA410674.

SEQ ID NO: 4101 represents the cDNA sequence for clone AA410679.

SEQ ID NO: 4102 represents the cDNA sequence for clone AA410701.

SEQ ID NO: 4103 represents the cDNA sequence for clone AA410703.

SEQ ID NO: 4104 represents the cDNA sequence for clone AA410687.

SEQ ID NO: 4105 represents the cDNA sequence for clone AA410719.

SEQ ID NO: 4106 represents the cDNA sequence for clone AA410720.

SEQ ID NO: 4107 represents the cDNA sequence for clone AA410721.

SEQ ID NO: 4108 represents the cDNA sequence for clone AA410722.

SEQ ID NO: 4109 represents the cDNA sequence for clone AA410732.

SEQ ID NO: 4110 represents the cDNA sequence for clone AA410734.

SEQ ID NO: 4111 represents the cDNA sequence for clone AA410740.

SEQ ID NO: 4112 represents the cDNA sequence for clone AA410627.

SEQ ID NO: 4113 represents the cDNA sequence for clone AA410542.

SEQ ID NO: 4114 represents the cDNA sequence for clone AA410549.

SEQ ID NO: 4115 represents the cDNA sequence for clone AA410749.

SEQ ID NO: 4116 represents the cDNA sequence for clone AA410769.

SEQ ID NO: 4117 represents the cDNA sequence for clone AA410776.

SEQ ID NO: 4118 represents the cDNA sequence for clone AA410778.

SEQ ID NO: 4119 represents the cDNA sequence for clone AA410786.

SEQ ID NO: 4120 represents the cDNA sequence for clone AA410792.

SEQ ID NO: 4121 represents the cDNA sequence for clone AA410804.

SEQ ID NO: 4122 represents the cDNA sequence for clone AA410699.

SEQ ID NO: 4123 represents the cDNA sequence for clone AA410761.

SEQ ID NO: 4124 represents the cDNA sequence for clone AA410832.

SEQ ID NO: 4125 represents the cDNA sequence for clone AA410834.

SEQ ID NO: 4126 represents the cDNA sequence for clone AA410835.

SEQ ID NO: 4127 represents the cDNA sequence for clone AA410838.

SEQ ID NO: 4128 represents the cDNA sequence for clone AA410849.

SEQ ID NO: 4129 represents the cDNA sequence for clone AA410853.

SEQ ID NO: 4130 represents the cDNA sequence for clone AA410860.

SEQ ID NO: 4131 represents the cDNA sequence for clone AA410864.

SEQ ID NO: 4132 represents the cDNA sequence for clone AA410820.

SEQ ID NO: 4133 represents the cDNA sequence for clone AA410822.

SEQ ID NO: 4134 represents the cDNA sequence for clone AA410823.

SEQ ID NO: 4135 represents the cDNA sequence for clone AA410825.

SEQ ID NO: 4136 represents the cDNA sequence for clone AA410828.

SEQ ID NO: 4137 represents the cDNA sequence for clone AA410829.

SEQ ID NO: 4138 represents the cDNA sequence for clone AA410752.

SEQ ID NO: 4139 represents the cDNA sequence for clone AA410756.

SEQ ID NO: 4140 represents the cDNA sequence for clone AA410878.

SEQ ID NO: 4141 represents the cDNA sequence for clone AA410902.

SEQ ID NO: 4142 represents the cDNA sequence for clone AA410903.

SEQ ID NO: 4143 represents the cDNA sequence for clone AA410909.

SEQ ID NO: 4144 represents the cDNA sequence for clone AA410910.

SEQ ID NO: 4145 represents the cDNA sequence for clone AA410911.

SEQ ID NO: 4146 represents the cDNA sequence for clone AA410922.

SEQ ID NO: 4147 represents the cDNA sequence for clone AA410924.

SEQ ID NO: 4148 represents the cDNA sequence for clone AA410932.

SEQ ID NO: 4149 represents the cDNA sequence for clone AA410933.

SEQ ID NO: 4150 represents the cDNA sequence for clone AA410865.

SEQ ID NO: 4151 represents the cDNA sequence for clone AA410873.

SEQ ID NO: 4152 represents the cDNA sequence for clone AA410708.

SEQ ID NO: 4153 represents the cDNA sequence for clone AA410709.

SEQ ID NO: 4154 represents the cDNA sequence for clone AA410711.

SEQ ID NO: 4155 represents the cDNA sequence for clone AA410947.

SEQ ID NO: 4156 represents the cDNA sequence for clone AA410948.

SEQ ID NO: 4157 represents the cDNA sequence for clone AA410949.

SEQ ID NO: 4158 represents the cDNA sequence for clone AA410977.

SEQ ID NO: 4159 represents the cDNA sequence for clone AA411012.

SEQ ID NO: 4160 represents the cDNA sequence for clone AA411016.

SEQ ID NO: 4161 represents the cDNA sequence for clone AA411046.

SEQ ID NO: 4162 represents the cDNA sequence for clone AA411021.

SEQ ID NO: 4163 represents the cDNA sequence for clone AA410941.

SEQ ID NO: 4164 represents the cDNA sequence for clone AA410942.

SEQ ID NO: 4165 represents the cDNA sequence for clone AA410943.

SEQ ID NO: 4166 represents the cDNA sequence for clone AA411064.

SEQ ID NO: 4167 represents the cDNA sequence for clone AA411077.

SEQ ID NO: 4168 represents the cDNA sequence for clone AA411080.

SEQ ID NO: 4169 represents the cDNA sequence for clone AA411083.

SEQ ID NO: 4170 represents the cDNA sequence for clone AA411101.

SEQ ID NO: 4171 represents the cDNA sequence for clone AA411102.

SEQ ID NO: 4172 represents the cDNA sequence for clone AA411085.

SEQ ID NO: 4173 represents the cDNA sequence for clone AA411117.

SEQ ID NO: 4174 represents the cDNA sequence for clone AA411118.

SEQ ID NO: 4175 represents the cDNA sequence for clone AA411119.

SEQ ID NO: 4176 represents the cDNA sequence for clone AA411126.

SEQ ID NO: 4177 represents the cDNA sequence for clone AA411128.

SEQ ID NO: 4178 represents the cDNA sequence for clone AA411133.

SEQ ID NO: 4179 represents the cDNA sequence for clone AA411138.

SEQ ID NO: 4180 represents the cDNA sequence for clone AA411140.

SEQ ID NO: 4181 represents the cDNA sequence for clone AA411142.

SEQ ID NO: 4182 represents the cDNA sequence for clone AA410963.

SEQ ID NO: 4183 represents the cDNA sequence for clone AA411110.

SEQ ID NO: 4184 represents the cDNA sequence for clone AA411143.

SEQ ID NO: 4185 represents the cDNA sequence for clone AA411144.

SEQ ID NO: 4186 represents the cDNA sequence for clone AA411155.

SEQ ID NO: 4187 represents the cDNA sequence for clone AA411156.

SEQ ID NO: 4188 represents the cDNA sequence for clone AA411161.

SEQ ID NO: 4189 represents the cDNA sequence for clone AA411162.

SEQ ID NO: 4190 represents the cDNA sequence for clone AA411171.

SEQ ID NO: 4191 represents the cDNA sequence for clone AA411175.

SEQ ID NO: 4192 represents the cDNA sequence for clone AA411185.

SEQ ID NO: 4193 represents the cDNA sequence for clone AA418995.

SEQ ID NO: 4194 represents the cDNA sequence for clone AA418999.

SEQ ID NO: 4195 represents the cDNA sequence for clone AA419018.

SEQ ID NO: 4196 represents the cDNA sequence for clone AA419022.

SEQ ID NO: 4197 represents the cDNA sequence for clone AA419042.

SEQ ID NO: 4198 represents the cDNA sequence for clone AA419043.

SEQ ID NO: 4199 represents the cDNA sequence for clone AA419044.

SEQ ID NO: 4200 represents the cDNA sequence for clone AA419050.

SEQ ID NO: 4201 represents the cDNA sequence for clone AA419006.

SEQ ID NO: 4202 represents the cDNA sequence for clone AA419057.

SEQ ID NO: 4203 represents the cDNA sequence for clone AA419077.

SEQ ID NO: 4204 represents the cDNA sequence for clone AA419010.

SEQ ID NO: 4205 represents the cDNA sequence for clone AA419074.

SEQ ID NO: 4206 represents the cDNA sequence for clone AA419088.

SEQ ID NO: 4207 represents the cDNA sequence for clone AA419092.

SEQ ID NO: 4208 represents the cDNA sequence for clone AA419112.

SEQ ID NO: 4209 represents the cDNA sequence for clone AA419116.

SEQ ID NO: 4210 represents the cDNA sequence for clone AA419121.

SEQ ID NO: 4211 represents the cDNA sequence for clone AA419122.

SEQ ID NO: 4212 represents the cDNA sequence for clone AA419124.

SEQ ID NO: 4213 represents the cDNA sequence for clone AA419125.

SEQ ID NO: 4214 represents the cDNA sequence for clone AA419126.

SEQ ID NO: 4215 represents the cDNA sequence for clone AA419028.

SEQ ID NO: 4216 represents the cDNA sequence for clone AA419029.

SEQ ID NO: 4217 represents the cDNA sequence for clone AA419105.

SEQ ID NO: 4218 represents the cDNA sequence for clone AA419061.

SEQ ID NO: 4219 represents the cDNA sequence for clone AA419064.

SEQ ID NO: 4220 represents the cDNA sequence for clone AA419147.

SEQ ID NO: 4221 represents the cDNA sequence for clone AA419153.

SEQ ID NO: 4222 represents the cDNA sequence for clone AA419158.

SEQ ID NO: 4223 represents the cDNA sequence for clone AA419159.

SEQ ID NO: 4224 represents the cDNA sequence for clone AA419162.

SEQ ID NO: 4225 represents the cDNA sequence for clone AA419184.

SEQ ID NO: 4226 represents the cDNA sequence for clone AA419185.

SEQ ID NO: 4227 represents the cDNA sequence for clone AA419187.

SEQ ID NO: 4228 represents the cDNA sequence for clone AA419195.

SEQ ID NO: 4229 represents the cDNA sequence for clone AA419101.

SEQ ID NO: 4230 represents the cDNA sequence for clone AA419173.

SEQ ID NO: 4231 represents the cDNA sequence for clone AA419179.

SEQ ID NO: 4232 represents the cDNA sequence for clone AA419198.

SEQ ID NO: 4233 represents the cDNA sequence for clone AA419204.

SEQ ID NO: 4234 represents the cDNA sequence for clone AA419205.

SEQ ID NO: 4235 represents the cDNA sequence for clone AA419144.

SEQ ID NO: 4236 represents the cDNA sequence for clone AA419216.

SEQ ID NO: 4237 represents the cDNA sequence for clone AA419217.

SEQ ID NO: 4238 represents the cDNA sequence for clone AA419219.

SEQ ID NO: 4239 represents the cDNA sequence for clone AA419226.

SEQ ID NO: 4240 represents the cDNA sequence for clone AA419229.

SEQ ID NO: 4241 represents the cDNA sequence for clone AA419254.

SEQ ID NO: 4242 represents the cDNA sequence for clone AA419258.

SEQ ID NO: 4243 represents the cDNA sequence for clone AA419261.

SEQ ID NO: 4244 represents the cDNA sequence for clone AA419262.

SEQ ID NO: 4245 represents the cDNA sequence for clone AA419264.

SEQ ID NO: 4246 represents the cDNA sequence for clone AA419267.

SEQ ID NO: 4247 represents the cDNA sequence for clone AA419271.

SEQ ID NO: 4248 represents the cDNA sequence for clone AA419281.

SEQ ID NO: 4249 represents the cDNA sequence for clone AA419234.

SEQ ID NO: 4250 represents the cDNA sequence for clone AA419237.

SEQ ID NO: 4251 represents the cDNA sequence for clone AA419214.

SEQ ID NO: 4252 represents the cDNA sequence for clone AA419287.

SEQ ID NO: 4253 represents the cDNA sequence for clone AA419289.

SEQ ID NO: 4254 represents the cDNA sequence for clone AA419290.

SEQ ID NO: 4255 represents the cDNA sequence for clone AA419310.

SEQ ID NO: 4256 represents the cDNA sequence for clone AA419312.

SEQ ID NO: 4257 represents the cDNA sequence for clone AA419313.

SEQ ID NO: 4258 represents the cDNA sequence for clone AA419331.

SEQ ID NO: 4259 represents the cDNA sequence for clone AA419335.

SEQ ID NO: 4260 represents the cDNA sequence for clone AA419336.

SEQ ID NO: 4261 represents the cDNA sequence for clone AA419164.

SEQ ID NO: 4262 represents the cDNA sequence for clone AA419165.

SEQ ID NO: 4263 represents the cDNA sequence for clone AA419166.

SEQ ID NO: 4264 represents the cDNA sequence for clone AA419167.

SEQ ID NO: 4265 represents the cDNA sequence for clone AA419248.

SEQ ID NO: 4266 represents the cDNA sequence for clone AA419298.

SEQ ID NO: 4267 represents the cDNA sequence for clone AA419354.

SEQ ID NO: 4268 represents the cDNA sequence for clone AA419369.

SEQ ID NO: 4269 represents the cDNA sequence for clone AA419373.

SEQ ID NO: 4270 represents the cDNA sequence for clone AA419376.

SEQ ID NO: 4271 represents the cDNA sequence for clone AA419302.

SEQ ID NO: 4272 represents the cDNA sequence for clone AA419304.

SEQ ID NO: 4273 represents the cDNA sequence for clone AA419306.

SEQ ID NO: 4274 represents the cDNA sequence for clone AA419343.

SEQ ID NO: 4275 represents the cDNA sequence for clone AA421211.

SEQ ID NO: 4276 represents the cDNA sequence for clone AA421097.

SEQ ID NO: 4277 represents the cDNA sequence for clone AA421098.

SEQ ID NO: 4278 represents the cDNA sequence for clone AA421036.

SEQ ID NO: 4279 represents the cDNA sequence for clone AA421037.

SEQ ID NO: 4280 represents the cDNA sequence for clone AA421233.

SEQ ID NO: 4281 represents the cDNA sequence for clone AA421251.

SEQ ID NO: 4282 represents the cDNA sequence for clone AA421254.

SEQ ID NO: 4283 represents the cDNA sequence for clone AA421255.

SEQ ID NO: 4284 represents the cDNA sequence for clone AA421246.

SEQ ID NO: 4285 represents the cDNA sequence for clone AA421247.

SEQ ID NO: 4286 represents the cDNA sequence for clone AA421328.

SEQ ID NO: 4287 represents the cDNA sequence for clone AA421334.

SEQ ID NO: 4288 represents the cDNA sequence for clone AA421335.

SEQ ID NO: 4289 represents the cDNA sequence for clone AA421350.

SEQ ID NO: 4290 represents the cDNA sequence for clone AA421353.

SEQ ID NO: 4291 represents the cDNA sequence for clone AA421414.

SEQ ID NO: 4292 represents the cDNA sequence for clone AA421416.

SEQ ID NO: 4293 represents the cDNA sequence for clone AA421417.

SEQ ID NO: 4294 represents the cDNA sequence for clone AA421424.

SEQ ID NO: 4295 represents the cDNA sequence for clone AA421427.

SEQ ID NO: 4296 represents the cDNA sequence for clone AA421431.

SEQ ID NO: 4297 represents the cDNA sequence for clone AA421175.

SEQ ID NO: 4298 represents the cDNA sequence for clone AA421176.

SEQ ID NO: 4299 represents the cDNA sequence for clone AA421182.

SEQ ID NO: 4300 represents the cDNA sequence for clone AA421183.

SEQ ID NO: 4301 represents the cDNA sequence for clone AA421383.

SEQ ID NO: 4302 represents the cDNA sequence for clone AA421385.

SEQ ID NO: 4303 represents the cDNA sequence for clone AA421388.

SEQ ID NO: 4304 represents the cDNA sequence for clone AA421389.

SEQ ID NO: 4305 represents the cDNA sequence for clone AA421434.

SEQ ID NO: 4306 represents the cDNA sequence for clone AA421438.

SEQ ID NO: 4307 represents the cDNA sequence for clone AA421440.

SEQ ID NO: 4308 represents the cDNA sequence for clone AA421462.

SEQ ID NO: 4309 represents the cDNA sequence for clone AA421594.

SEQ ID NO: 4310 represents the cDNA sequence for clone AA421596.

SEQ ID NO: 4311 represents the cDNA sequence for clone AA421599.

SEQ ID NO: 4312 represents the cDNA sequence for clone AA421601.

SEQ ID NO: 4313 represents the cDNA sequence for clone AA421659.

SEQ ID NO: 4314 represents the cDNA sequence for clone AA421685.

SEQ ID NO: 4315 represents the cDNA sequence for clone AA421695.

SEQ ID NO: 4316 represents the cDNA sequence for clone AA421668.

SEQ ID NO: 4317 represents the cDNA sequence for clone AA421710.

SEQ ID NO: 4318 represents the cDNA sequence for clone AA421712.

SEQ ID NO: 4319 represents the cDNA sequence for clone AA421673.

SEQ ID NO: 4320 represents the cDNA sequence for clone AA421674.

SEQ ID NO: 4321 represents the cDNA sequence for clone AA421679.

SEQ ID NO: 4322 represents the cDNA sequence for clone AA421603.

SEQ ID NO: 4323 represents the cDNA sequence for clone AA421607.

SEQ ID NO: 4324 represents the cDNA sequence for clone AA421784.

SEQ ID NO: 4325 represents the cDNA sequence for clone AA421785.

SEQ ID NO: 4326 represents the cDNA sequence for clone AA421790.

SEQ ID NO: 4327 represents the cDNA sequence for clone AA421791.

SEQ ID NO: 4328 represents the cDNA sequence for clone AA421813.

SEQ ID NO: 4329 represents the cDNA sequence for clone AA421816.

SEQ ID NO: 4330 represents the cDNA sequence for clone AA421817.

SEQ ID NO: 4331 represents the cDNA sequence for clone AA421819.

SEQ ID NO: 4332 represents the cDNA sequence for clone AA421733.

SEQ ID NO: 4333 represents the cDNA sequence for clone AA421738.

SEQ ID NO: 4334 represents the cDNA sequence for clone AA421853.

SEQ ID NO: 4335 represents the cDNA sequence for clone AA421857.

SEQ ID NO: 4336 represents the cDNA sequence for clone AA421880.

SEQ ID NO: 4337 represents the cDNA sequence for clone AA421888.

SEQ ID NO: 4338 represents the cDNA sequence for clone AA421893.

SEQ ID NO: 4339 represents the cDNA sequence for clone AA421896.

SEQ ID NO: 4340 represents the cDNA sequence for clone AA421900.

SEQ ID NO: 4341 represents the cDNA sequence for clone AA421908.

SEQ ID NO: 4342 represents the cDNA sequence for clone AA421909.

SEQ ID NO: 4343 represents the cDNA sequence for clone AA421722.

SEQ ID NO: 4344 represents the cDNA sequence for clone AA421724.

SEQ ID NO: 4345 represents the cDNA sequence for clone AA421725.

SEQ ID NO: 4346 represents the cDNA sequence for clone AA421727.

SEQ ID NO: 4347 represents the cDNA sequence for clone AA421728.

SEQ ID NO: 4348 represents the cDNA sequence for clone AA421922.

SEQ ID NO: 4349 represents the cDNA sequence for clone AA421923.

SEQ ID NO: 4350 represents the cDNA sequence for clone AA421924.

SEQ ID NO: 4351 represents the cDNA sequence for clone AA421973.

SEQ ID NO: 4352 represents the cDNA sequence for clone AA421974.

SEQ ID NO: 4353 represents the cDNA sequence for clone AA422036.

SEQ ID NO: 4354 represents the cDNA sequence for clone AA422040.

SEQ ID NO: 4355 represents the cDNA sequence for clone AA422044.

SEQ ID NO: 4356 represents the cDNA sequence for clone AA422048.

SEQ ID NO: 4357 represents the cDNA sequence for clone AA422052.

SEQ ID NO: 4358 represents the cDNA sequence for clone AA422056.

SEQ ID NO: 4359 represents the cDNA sequence for clone AA421865.

SEQ ID NO: 4360 represents the cDNA sequence for clone AA421867.

SEQ ID NO: 4361 represents the cDNA sequence for clone AA422006.

SEQ ID NO: 4362 represents the cDNA sequence for clone AA422007.

SEQ ID NO: 4363 represents the cDNA sequence for clone AA422008.

SEQ ID NO: 4364 represents the cDNA sequence for clone AA422127.

SEQ ID NO: 4365 represents the cDNA sequence for clone AA422151.

SEQ ID NO: 4366 represents the cDNA sequence for clone AA422153.

SEQ ID NO: 4367 represents the cDNA sequence for clone AA422163.

SEQ ID NO: 4368 represents the cDNA sequence for clone AA422165.

SEQ ID NO: 4369 represents the cDNA sequence for clone AA422166.

SEQ ID NO: 4370 represents the cDNA sequence for clone AA422167.

SEQ ID NO: 4371 represents the cDNA sequence for clone AA422168.

SEQ ID NO: 4372 represents the cDNA sequence for clone AA421998.

SEQ ID NO: 4373 represents the cDNA sequence for clone AA421999.

SEQ ID NO: 4374 represents the cDNA sequence for clone AA422179.

SEQ ID NO: 4375 represents the cDNA sequence for clone AA422183.

SEQ ID NO: 4376 represents the cDNA sequence for clone AA424696.

SEQ ID NO: 4377 represents the cDNA sequence for clone AA424700.

SEQ ID NO: 4378 represents the cDNA sequence for clone AA424701.

SEQ ID NO: 4379 represents the cDNA sequence for clone AA424706.

SEQ ID NO: 4380 represents the cDNA sequence for clone AA423796.

SEQ ID NO: 4381 represents the cDNA sequence for clone AA423797.

SEQ ID NO: 4382 represents the cDNA sequence for clone AA423798.

SEQ ID NO: 4383 represents the cDNA sequence for clone AA423813.

SEQ ID NO: 4384 represents the cDNA sequence for clone AA423823.

SEQ ID NO: 4385 represents the cDNA sequence for clone AA425633.

SEQ ID NO: 4386 represents the cDNA sequence for clone AA425730.

SEQ ID NO: 4387 represents the cDNA sequence for clone AA425732.

SEQ ID NO: 4388 represents the cDNA sequence for clone AA425733.

SEQ ID NO: 4389 represents the cDNA sequence for clone AA425734.

SEQ ID NO: 4390 represents the cDNA sequence for clone AA425735.

SEQ ID NO: 4391 represents the cDNA sequence for clone AA425787.

SEQ ID NO: 4392 represents the cDNA sequence for clone AA425867.

SEQ ID NO: 4393 represents the cDNA sequence for clone AA425870.

SEQ ID NO: 4394 represents the cDNA sequence for clone AA425873.

SEQ ID NO: 4395 represents the cDNA sequence for clone AA426095.

SEQ ID NO: 4396 represents the cDNA sequence for clone AA426096.

SEQ ID NO: 4397 represents the cDNA sequence for clone AA426446.

SEQ ID NO: 4398 represents the cDNA sequence for clone AA426451.

SEQ ID NO: 4399 represents the cDNA sequence for clone AA426540.

SEQ ID NO: 4400 represents the cDNA sequence for clone AA426546.

SEQ ID NO: 4401 represents the cDNA sequence for clone AA426547.

SEQ ID NO: 4402 represents the cDNA sequence for clone AA426615.

SEQ ID NO: 4403 represents the cDNA sequence for clone AA426617.

SEQ ID NO: 4404 represents the cDNA sequence for clone AA426618.

SEQ ID NO: 4405 represents the cDNA sequence for clone AA426619.

SEQ ID NO: 4406 represents the cDNA sequence for clone AA426648.

SEQ ID NO: 4407 represents the cDNA sequence for clone AA426655.

SEQ ID NO: 4408 represents the cDNA sequence for clone AA426647.

SEQ ID NO: 4409 represents the cDNA sequence for clone AA424835.

SEQ ID NO: 4410 represents the cDNA sequence for clone AA424839.

SEQ ID NO: 4411 represents the cDNA sequence for clone AA424840.

SEQ ID NO: 4412 represents the cDNA sequence for clone AA424841.

SEQ ID NO: 4413 represents the cDNA sequence for clone AA424890.

SEQ ID NO: 4414 represents the cDNA sequence for clone AA424898.

SEQ ID NO: 4415 represents the cDNA sequence for clone AA424929.

SEQ ID NO: 4416 represents the cDNA sequence for clone AA424932.

SEQ ID NO: 4417 represents the cDNA sequence for clone AA425119.

SEQ ID NO: 4418 represents the cDNA sequence for clone AA425120.

SEQ ID NO: 4419 represents the cDNA sequence for clone AA425121.

SEQ ID NO: 4420 represents the cDNA sequence for clone AA425123.

SEQ ID NO: 4421 represents the cDNA sequence for clone AA425327.

SEQ ID NO: 4422 represents the cDNA sequence for clone AA425329.

SEQ ID NO: 4423 represents the cDNA sequence for clone AA425331.

SEQ ID NO: 4424 represents the cDNA sequence for clone AA425334.

SEQ ID NO: 4425 represents the cDNA sequence for clone AA425714.

SEQ ID NO: 4426 represents the cDNA sequence for clone AA425715.

SEQ ID NO: 4427 represents the cDNA sequence for clone AA425718.

SEQ ID NO: 4428 represents the cDNA sequence for clone AA425719.

SEQ ID NO: 4429 represents the cDNA sequence for clone AA426079.

SEQ ID NO: 4430 represents the cDNA sequence for clone AA426080.

SEQ ID NO: 4431 represents the cDNA sequence for clone AA426081.

SEQ ID NO: 4432 represents the cDNA sequence for clone AA426082.

SEQ ID NO: 4433 represents the cDNA sequence for clone AA426264.

SEQ ID NO: 4434 represents the cDNA sequence for clone AA426267.

SEQ ID NO: 4435 represents the cDNA sequence for clone AA426270.

SEQ ID NO: 4436 represents the cDNA sequence for clone AA426213.

SEQ ID NO: 4437 represents the cDNA sequence for clone AA426214.

SEQ ID NO: 4438 represents the cDNA sequence for clone AA426218.

SEQ ID NO: 4439 represents the cDNA sequence for clone AA425796.

SEQ ID NO: 4440 represents the cDNA sequence for clone AA425820.

SEQ ID NO: 4441 represents the cDNA sequence for clone AA425848.

SEQ ID NO: 4442 represents the cDNA sequence for clone AA426150.

SEQ ID NO: 4443 represents the cDNA sequence for clone AA425834.

SEQ ID NO: 4444 represents the cDNA sequence for clone AA425837.

SEQ ID NO: 4445 represents the cDNA sequence for clone AA425840.

SEQ ID NO: 4446 represents the cDNA sequence for clone AA425887.

SEQ ID NO: 4447 represents the cDNA sequence for clone AA425888.

SEQ ID NO: 4448 represents the cDNA sequence for clone AA425891.

SEQ ID NO: 4449 represents the cDNA sequence for clone AA425892.

SEQ ID NO: 4450 represents the cDNA sequence for clone AA425922.

SEQ ID NO: 4451 represents the cDNA sequence for clone AA425924.

SEQ ID NO: 4452 represents the cDNA sequence for clone AA425925.

SEQ ID NO: 4453 represents the cDNA sequence for clone AA425927.

SEQ ID NO: 4454 represents the cDNA sequence for clone AA425928.

SEQ ID NO: 4455 represents the cDNA sequence for clone AA425930.

SEQ ID NO: 4456 represents the cDNA sequence for clone AA425934.

SEQ ID NO: 4457 represents the cDNA sequence for clone AA425895.

SEQ ID NO: 4458 represents the cDNA sequence for clone AA425900.

SEQ ID NO: 4459 represents the cDNA sequence for clone AA425966.

SEQ ID NO: 4460 represents the cDNA sequence for clone AA425970.

SEQ ID NO: 4461 represents the cDNA sequence for clone AA425973.

SEQ ID NO: 4462 represents the cDNA sequence for clone AA425984.

SEQ ID NO: 4463 represents the cDNA sequence for clone AA425985.

SEQ ID NO: 4464 represents the cDNA sequence for clone AA425986.

SEQ ID NO: 4465 represents the cDNA sequence for clone AA425988.

SEQ ID NO: 4466 represents the cDNA sequence for clone AA425991.

SEQ ID NO: 4467 represents the cDNA sequence for clone AA425998.

SEQ ID NO: 4468 represents the cDNA sequence for clone AA426001.

SEQ ID NO: 4469 represents the cDNA sequence for clone AA426281.

SEQ ID NO: 4470 represents the cDNA sequence for clone AA426284.

SEQ ID NO: 4471 represents the cDNA sequence for clone AA426287.

SEQ ID NO: 4472 represents the cDNA sequence for clone AA428275.

SEQ ID NO: 4473 represents the cDNA sequence for clone AA428276.

SEQ ID NO: 4474 represents the cDNA sequence for clone AA428300.

SEQ ID NO: 4475 represents the cDNA sequence for clone AA428305.

SEQ ID NO: 4476 represents the cDNA sequence for clone AA428306.

SEQ ID NO: 4477 represents the cDNA sequence for clone AA428294.

SEQ ID NO: 4478 represents the cDNA sequence for clone AA428295.

SEQ ID NO: 4479 represents the cDNA sequence for clone AA428321.

SEQ ID NO: 4480 represents the cDNA sequence for clone AA428329.

SEQ ID NO: 4481 represents the cDNA sequence for clone AA428330.

SEQ ID NO: 4482 represents the cDNA sequence for clone AA428334.

SEQ ID NO: 4483 represents the cDNA sequence for clone AA428340.

SEQ ID NO: 4484 represents the cDNA sequence for clone AA428341.

SEQ ID NO: 4485 represents the cDNA sequence for clone AA428350.

SEQ ID NO: 4486 represents the cDNA sequence for clone AA428369.

SEQ ID NO: 4487 represents the cDNA sequence for clone AA428370.

SEQ ID NO: 4488 represents the cDNA sequence for clone AA428373.

SEQ ID NO: 4489 represents the cDNA sequence for clone AA428400.

SEQ ID NO: 4490 represents the cDNA sequence for clone AA428402.

SEQ ID NO: 4491 represents the cDNA sequence for clone AA428403.

SEQ ID NO: 4492 represents the cDNA sequence for clone AA428424.

SEQ ID NO: 4493 represents the cDNA sequence for clone AA428425.

SEQ ID NO: 4494 represents the cDNA sequence for clone AA428426.

SEQ ID NO: 4495 represents the cDNA sequence for clone AA428432.

SEQ ID NO: 4496 represents the cDNA sequence for clone AA428434.

SEQ ID NO: 4497 represents the cDNA sequence for clone AA428437.

SEQ ID NO: 4498 represents the cDNA sequence for clone AA428441.

SEQ ID NO: 4499 represents the cDNA sequence for clone AA428442.

SEQ ID NO: 4500 represents the cDNA sequence for clone AA428446.

SEQ ID NO: 4501 represents the cDNA sequence for clone AA428666.

SEQ ID NO: 4502 represents the cDNA sequence for clone AA428671.

SEQ ID NO: 4503 represents the cDNA sequence for clone AA428692.

SEQ ID NO: 4504 represents the cDNA sequence for clone AA428694.

SEQ ID NO: 4505 represents the cDNA sequence for clone AA428695.

SEQ ID NO: 4506 represents the cDNA sequence for clone AA428698.

SEQ ID NO: 4507 represents the cDNA sequence for clone AA428700.

SEQ ID NO: 4508 represents the cDNA sequence for clone AA428705.

SEQ ID NO: 4509 represents the cDNA sequence for clone AA428707.

SEQ ID NO: 4510 represents the cDNA sequence for clone AA428715.

SEQ ID NO: 4511 represents the cDNA sequence for clone AA428718.

SEQ ID NO: 4512 represents the cDNA sequence for clone AA428721.

SEQ ID NO: 4513 represents the cDNA sequence for clone AA428722.

SEQ ID NO: 4514 represents the cDNA sequence for clone AA428727.

SEQ ID NO: 4515 represents the cDNA sequence for clone AA428728.

SEQ ID NO: 4516 represents the cDNA sequence for clone AA428731.

SEQ ID NO: 4517 represents the cDNA sequence for clone AA428738.

SEQ ID NO: 4518 represents the cDNA sequence for clone AA428743.

SEQ ID NO: 4519 represents the cDNA sequence for clone AA428748.

SEQ ID NO: 4520 represents the cDNA sequence for clone AA428769.

SEQ ID NO: 4521 represents the cDNA sequence for clone AA428770.

SEQ ID NO: 4522 represents the cDNA sequence for clone AA428771.

SEQ ID NO: 4523 represents the cDNA sequence for clone AA428790.

SEQ ID NO: 4524 represents the cDNA sequence for clone AA428791.

SEQ ID NO: 4525 represents the cDNA sequence for clone AA428792.

SEQ ID NO: 4526 represents the cDNA sequence for clone AA428794.

SEQ ID NO: 4527 represents the cDNA sequence for clone AA428796.

SEQ ID NO: 4528 represents the cDNA sequence for clone AA428797.

SEQ ID NO: 4529 represents the cDNA sequence for clone AA428798.

SEQ ID NO: 4530 represents the cDNA sequence for clone AA428799.

SEQ ID NO: 4531 represents the cDNA sequence for clone AA428806.

SEQ ID NO: 4532 represents the cDNA sequence for clone AA428816.

SEQ ID NO: 4533 represents the cDNA sequence for clone AA428758.

SEQ ID NO: 4534 represents the cDNA sequence for clone AA428761.

SEQ ID NO: 4535 represents the cDNA sequence for clone AA428765.

SEQ ID NO: 4536 represents the cDNA sequence for clone AA428821.

SEQ ID NO: 4537 represents the cDNA sequence for clone AA428827.

SEQ ID NO: 4538 represents the cDNA sequence for clone AA428854.

SEQ ID NO: 4539 represents the cDNA sequence for clone AA428855.

SEQ ID NO: 4540 represents the cDNA sequence for clone AA428859.

SEQ ID NO: 4541 represents the cDNA sequence for clone AA428756.

SEQ ID NO: 4542 represents the cDNA sequence for clone AA428837.

SEQ ID NO: 4543 represents the cDNA sequence for clone AA428838.

SEQ ID NO: 4544 represents the cDNA sequence for clone AA428839.

SEQ ID NO: 4545 represents the cDNA sequence for clone AA428881.

SEQ ID NO: 4546 represents the cDNA sequence for clone AA428886.

SEQ ID NO: 4547 represents the cDNA sequence for clone AA428776.

SEQ ID NO: 4548 represents the cDNA sequence for clone AA428781.

SEQ ID NO: 4549 represents the cDNA sequence for clone AA428784.

SEQ ID NO: 4550 represents the cDNA sequence for clone AA428899.

SEQ ID NO: 4551 represents the cDNA sequence for clone AA428960.

SEQ ID NO: 4552 represents the cDNA sequence for clone AA428964.

SEQ ID NO: 4553 represents the cDNA sequence for clone AA428965.

SEQ ID NO: 4554 represents the cDNA sequence for clone AA428938.

SEQ ID NO: 4555 represents the cDNA sequence for clone AA428974.

SEQ ID NO: 4556 represents the cDNA sequence for clone AA428982.

SEQ ID NO: 4557 represents the cDNA sequence for clone AA428991.

SEQ ID NO: 4558 represents the cDNA sequence for clone AA428992.

SEQ ID NO: 4559 represents the cDNA sequence for clone AA428994.

SEQ ID NO: 4560 represents the cDNA sequence for clone AA429021.

SEQ ID NO: 4561 represents the cDNA sequence for clone AA429022.

SEQ ID NO: 4562 represents the cDNA sequence for clone AA428957.

SEQ ID NO: 4563 represents the cDNA sequence for clone AA429040.

SEQ ID NO: 4564 represents the cDNA sequence for clone AA428972.

SEQ ID NO: 4565 represents the cDNA sequence for clone AA429052.

SEQ ID NO: 4566 represents the cDNA sequence for clone AA429066.

SEQ ID NO: 4567 represents the cDNA sequence for clone AA429068.

SEQ ID NO: 4568 represents the cDNA sequence for clone AA429069.

SEQ ID NO: 4569 represents the cDNA sequence for clone AA429072.

SEQ ID NO: 4570 represents the cDNA sequence for clone AA429094.

SEQ ID NO: 4571 represents the cDNA sequence for clone AA429095.

SEQ ID NO: 4572 represents the cDNA sequence for clone AA429102.

SEQ ID NO: 4573 represents the cDNA sequence for clone AA429127.

SEQ ID NO: 4574 represents the cDNA sequence for clone AA429128.

SEQ ID NO: 4575 represents the cDNA sequence for clone AA429131.

SEQ ID NO: 4576 represents the cDNA sequence for clone AA429119.

SEQ ID NO: 4577 represents the cDNA sequence for clone AA429123.

SEQ ID NO: 4578 represents the cDNA sequence for clone AA429124.

SEQ ID NO: 4579 represents the cDNA sequence for clone AA429146.

SEQ ID NO: 4580 represents the cDNA sequence for clone AA429147.

SEQ ID NO: 4581 represents the cDNA sequence for clone AA429168.

SEQ ID NO: 4582 represents the cDNA sequence for clone AA429169.

SEQ ID NO: 4583 represents the cDNA sequence for clone AA429170.

SEQ ID NO: 4584 represents the cDNA sequence for clone AA429172.

SEQ ID NO: 4585 represents the cDNA sequence for clone AA429173.

SEQ ID NO: 4586 represents the cDNA sequence for clone AA429175.

SEQ ID NO: 4587 represents the cDNA sequence for clone AA429179.

SEQ ID NO: 4588 represents the cDNA sequence for clone AA429180.

SEQ ID NO: 4589 represents the cDNA sequence for clone AA429181.

SEQ ID NO: 4590 represents the cDNA sequence for clone AA429152.

SEQ ID NO: 4591 represents the cDNA sequence for clone AA429154.

SEQ ID NO: 4592 represents the cDNA sequence for clone AA429156.

SEQ ID NO: 4593 represents the cDNA sequence for clone AA429157.

SEQ ID NO: 4594 represents the cDNA sequence for clone AA429158.

SEQ ID NO: 4595 represents the cDNA sequence for clone AA429199.

SEQ ID NO: 4596 represents the cDNA sequence for clone AA429205.

SEQ ID NO: 4597 represents the cDNA sequence for clone AA429223.

SEQ ID NO: 4598 represents the cDNA sequence for clone AA429163.

SEQ ID NO: 4599 represents the cDNA sequence for clone AA429167.

SEQ ID NO: 4600 represents the cDNA sequence for clone AA429251.

SEQ ID NO: 4601 represents the cDNA sequence for clone AA429212.

SEQ ID NO: 4602 represents the cDNA sequence for clone AA429215.

SEQ ID NO: 4603 represents the cDNA sequence for clone AA429262.

SEQ ID NO: 4604 represents the cDNA sequence for clone AA429266.

SEQ ID NO: 4605 represents the cDNA sequence for clone AA429269.

SEQ ID NO: 4606 represents the cDNA sequence for clone AA429273.

SEQ ID NO: 4607 represents the cDNA sequence for clone AA429254.

SEQ ID NO: 4608 represents the cDNA sequence for clone AA429257.

SEQ ID NO: 4609 represents the cDNA sequence for clone AA429345.

SEQ ID NO: 4610 represents the cDNA sequence for clone AA429351.

SEQ ID NO: 4611 represents the cDNA sequence for clone AA429357.

SEQ ID NO: 4612 represents the cDNA sequence for clone AA429362.

SEQ ID NO: 4613 represents the cDNA sequence for clone AA430357.

SEQ ID NO: 4614 represents the cDNA sequence for clone AA430358.

SEQ ID NO: 4615 represents the cDNA sequence for clone AA430366.

SEQ ID NO: 4616 represents the cDNA sequence for clone AA430336.

SEQ ID NO: 4617 represents the cDNA sequence for clone AA430338.

SEQ ID NO: 4618 represents the cDNA sequence for clone AA430395.

SEQ ID NO: 4619 represents the cDNA sequence for clone AA430400.

SEQ ID NO: 4620 represents the cDNA sequence for clone AA430401.

SEQ ID NO: 4621 represents the cDNA sequence for clone AA430431.

SEQ ID NO: 4622 represents the cDNA sequence for clone AA430432.

SEQ ID NO: 4623 represents the cDNA sequence for clone AA430435.

SEQ ID NO: 4624 represents the cDNA sequence for clone AA430441.

SEQ ID NO: 4625 represents the cDNA sequence for clone AA430332.

SEQ ID NO: 4626 represents the cDNA sequence for clone AA430417.

SEQ ID NO: 4627 represents the cDNA sequence for clone AA430471.

SEQ ID NO: 4628 represents the cDNA sequence for clone AA430389.

SEQ ID NO: 4629 represents the cDNA sequence for clone AA430390.

SEQ ID NO: 4630 represents the cDNA sequence for clone AA430466.

SEQ ID NO: 4631 represents the cDNA sequence for clone AA430467.

SEQ ID NO: 4632 represents the cDNA sequence for clone AA430484.

SEQ ID NO: 4633 represents the cDNA sequence for clone AA430486.

SEQ ID NO: 4634 represents the cDNA sequence for clone AA430514.

SEQ ID NO: 4635 represents the cDNA sequence for clone AA430515.

SEQ ID NO: 4636 represents the cDNA sequence for clone AA430517.

SEQ ID NO: 4637 represents the cDNA sequence for clone AA430519.

SEQ ID NO: 4638 represents the cDNA sequence for clone AA430522.

SEQ ID NO: 4639 represents the cDNA sequence for clone AA430523.

SEQ ID NO: 4640 represents the cDNA sequence for clone AA430504.

SEQ ID NO: 4641 represents the cDNA sequence for clone AA430510.

SEQ ID NO: 4642 represents the cDNA sequence for clone AA430511.

SEQ ID NO: 4643 represents the cDNA sequence for clone AA430512.

SEQ ID NO: 4644 represents the cDNA sequence for clone AA430404.

SEQ ID NO: 4645 represents the cDNA sequence for clone AA430407.

SEQ ID NO: 4646 represents the cDNA sequence for clone AA430410.

SEQ ID NO: 4647 represents the cDNA sequence for clone AA430538.

SEQ ID NO: 4648 represents the cDNA sequence for clone AA430539.

SEQ ID NO: 4649 represents the cDNA sequence for clone AA430543.

SEQ ID NO: 4650 represents the cDNA sequence for clone AA430550.

SEQ ID NO: 4651 represents the cDNA sequence for clone AA430552.

SEQ ID NO: 4652 represents the cDNA sequence for clone AA430553.

SEQ ID NO: 4653 represents the cDNA sequence for clone AA430577.

SEQ ID NO: 4654 represents the cDNA sequence for clone AA430457.

SEQ ID NO: 4655 represents the cDNA sequence for clone AA430459.

SEQ ID NO: 4656 represents the cDNA sequence for clone AA430461.

SEQ ID NO: 4657 represents the cDNA sequence for clone AA430564.

SEQ ID NO: 4658 represents the cDNA sequence for clone AA430569.

SEQ ID NO: 4659 represents the cDNA sequence for clone AA430570.

SEQ ID NO: 4660 represents the cDNA sequence for clone AA430571.

SEQ ID NO: 4661 represents the cDNA sequence for clone AA430558.

SEQ ID NO: 4662 represents the cDNA sequence for clone AA430562.

SEQ ID NO: 4663 represents the cDNA sequence for clone AA430531.

SEQ ID NO: 4664 represents the cDNA sequence for clone AA430536.

SEQ ID NO: 4665 represents the cDNA sequence for clone AA430537.

SEQ ID NO: 4666 represents the cDNA sequence for clone AA430600.

SEQ ID NO: 4667 represents the cDNA sequence for clone AA430602.

SEQ ID NO: 4668 represents the cDNA sequence for clone AA430604.

SEQ ID NO: 4669 represents the cDNA sequence for clone AA430608.

SEQ ID NO: 4670 represents the cDNA sequence for clone AA430646.

SEQ ID NO: 4671 represents the cDNA sequence for clone AA430647.

SEQ ID NO: 4672 represents the cDNA sequence for clone AA430651.

SEQ ID NO: 4673 represents the cDNA sequence for clone AA430636.

SEQ ID NO: 4674 represents the cDNA sequence for clone AA430639.

SEQ ID NO: 4675 represents the cDNA sequence for clone AA430640.

SEQ ID NO: 4676 represents the cDNA sequence for clone AA430641.

SEQ ID NO: 4677 represents the cDNA sequence for clone AA430682.

SEQ ID NO: 4678 represents the cDNA sequence for clone AA430669.

SEQ ID NO: 4679 represents the cDNA sequence for clone AA430672.

SEQ ID NO: 4680 represents the cDNA sequence for clone AA430701.

SEQ ID NO: 4681 represents the cDNA sequence for clone AA430722.

SEQ ID NO: 4682 represents the cDNA sequence for clone AA430726.

SEQ ID NO: 4683 represents the cDNA sequence for clone AA430727.

SEQ ID NO: 4684 represents the cDNA sequence for clone AA430737.

SEQ ID NO: 4685 represents the cDNA sequence for clone AA427360.

SEQ ID NO: 4686 represents the cDNA sequence for clone AA427363.

SEQ ID NO: 4687 represents the cDNA sequence for clone AA427408.

SEQ ID NO: 4688 represents the cDNA sequence for clone AA427409.

SEQ ID NO: 4689 represents the cDNA sequence for clone AA427414.

SEQ ID NO: 4690 represents the cDNA sequence for clone AA427439.

SEQ ID NO: 4691 represents the cDNA sequence for clone AA427440.

SEQ ID NO: 4692 represents the cDNA sequence for clone AA427451.

SEQ ID NO: 4693 represents the cDNA sequence for clone AA427452.

SEQ ID NO: 4694 represents the cDNA sequence for clone AA427453.

SEQ ID NO: 4695 represents the cDNA sequence for clone AA427457.

SEQ ID NO: 4696 represents the cDNA sequence for clone AA427466.

SEQ ID NO: 4697 represents the cDNA sequence for clone AA427467.

SEQ ID NO: 4698 represents the cDNA sequence for clone AA427470.

SEQ ID NO: 4699 represents the cDNA sequence for clone AA427473.

SEQ ID NO: 4700 represents the cDNA sequence for clone AA427496.

SEQ ID NO: 4701 represents the cDNA sequence for clone AA427497.

SEQ ID NO: 4702 represents the cDNA sequence for clone AA427498.

SEQ ID NO: 4703 represents the cDNA sequence for clone AA427499.

SEQ ID NO: 4704 represents the cDNA sequence for clone AA427502.

SEQ ID NO: 4705 represents the cDNA sequence for clone AA427503.

SEQ ID NO: 4706 represents the cDNA sequence for clone AA427481.

SEQ ID NO: 4707 represents the cDNA sequence for clone AA427483.

SEQ ID NO: 4708 represents the cDNA sequence for clone AA427445.

SEQ ID NO: 4709 represents the cDNA sequence for clone AA427449.

SEQ ID NO: 4710 represents the cDNA sequence for clone AA427450.

SEQ ID NO: 4711 represents the cDNA sequence for clone AA427512.

SEQ ID NO: 4712 represents the cDNA sequence for clone AA427513.

SEQ ID NO: 4713 represents the cDNA sequence for clone AA427487.

SEQ ID NO: 4714 represents the cDNA sequence for clone AA427490.

SEQ ID NO: 4715 represents the cDNA sequence for clone AA427555.

SEQ ID NO: 4716 represents the cDNA sequence for clone AA427569.

SEQ ID NO: 4717 represents the cDNA sequence for clone AA427571.

SEQ ID NO: 4718 represents the cDNA sequence for clone AA427557.

SEQ ID NO: 4719 represents the cDNA sequence for clone AA427558.

SEQ ID NO: 4720 represents the cDNA sequence for clone AA427562.

SEQ ID NO: 4721 represents the cDNA sequence for clone AA427584.

SEQ ID NO: 4722 represents the cDNA sequence for clone AA427588.

SEQ ID NO: 4723 represents the cDNA sequence for clone AA427595.

SEQ ID NO: 4724 represents the cDNA sequence for clone AA427597.

SEQ ID NO: 4725 represents the cDNA sequence for clone AA427617.

SEQ ID NO: 4726 represents the cDNA sequence for clone AA427622.

SEQ ID NO: 4727 represents the cDNA sequence for clone AA427629.

SEQ ID NO: 4728 represents the cDNA sequence for clone AA427646.

SEQ ID NO: 4729 represents the cDNA sequence for clone AA427666.

SEQ ID NO: 4730 represents the cDNA sequence for clone AA427667.

SEQ ID NO: 4731 represents the cDNA sequence for clone AA427671.

SEQ ID NO: 4732 represents the cDNA sequence for clone AA427680.

SEQ ID NO: 4733 represents the cDNA sequence for clone AA427712.

SEQ ID NO: 4734 represents the cDNA sequence for clone AA427653.

SEQ ID NO: 4735 represents the cDNA sequence for clone AA427655.

SEQ ID NO: 4736 represents the cDNA sequence for clone AA427660.

SEQ ID NO: 4737 represents the cDNA sequence for clone AA427661.

SEQ ID NO: 4738 represents the cDNA sequence for clone AA427662.

SEQ ID NO: 4739 represents the cDNA sequence for clone AA427724.

SEQ ID NO: 4740 represents the cDNA sequence for clone AA427725.

SEQ ID NO: 4741 represents the cDNA sequence for clone AA427730.

SEQ ID NO: 4742 represents the cDNA sequence for clone AA427731.

SEQ ID NO: 4743 represents the cDNA sequence for clone AA427744.

SEQ ID NO: 4744 represents the cDNA sequence for clone AA427745.

SEQ ID NO: 4745 represents the cDNA sequence for clone AA427746.

SEQ ID NO: 4746 represents the cDNA sequence for clone AA427701.

SEQ ID NO: 4747 represents the cDNA sequence for clone AA427706.

SEQ ID NO: 4748 represents the cDNA sequence for clone AA427750.

SEQ ID NO: 4749 represents the cDNA sequence for clone AA427767.

SEQ ID NO: 4750 represents the cDNA sequence for clone AA429370.

SEQ ID NO: 4751 represents the cDNA sequence for clone AA429371.

SEQ ID NO: 4752 represents the cDNA sequence for clone AA430746.

SEQ ID NO: 4753 represents the cDNA sequence for clone AA427400.

SEQ ID NO: 4754 represents the cDNA sequence for clone AA427402.

SEQ ID NO: 4755 represents the cDNA sequence for clone AA427404.

SEQ ID NO: 4756 represents the cDNA sequence for clone AA427721.

SEQ ID NO: 4757 represents the cDNA sequence for clone AA427722.

SEQ ID NO: 4758 represents the cDNA sequence for clone AA427872.

SEQ ID NO: 4759 represents the cDNA sequence for clone AA427975.

SEQ ID NO: 4760 represents the cDNA sequence for clone AA428088.

SEQ ID NO: 4761 represents the cDNA sequence for clone AA428091.

SEQ ID NO: 4762 represents the cDNA sequence for clone AA428092.

SEQ ID NO: 4763 represents the cDNA sequence for clone AA428171.

SEQ ID NO: 4764 represents the cDNA sequence for clone AA428250.

SEQ ID NO: 4765 represents the cDNA sequence for clone AA428252.

SEQ ID NO: 4766 represents the cDNA sequence for clone AA428253.

SEQ ID NO: 4767 represents the cDNA sequence for clone AA428413.

SEQ ID NO: 4768 represents the cDNA sequence for clone AA428414.

SEQ ID NO: 4769 represents the cDNA sequence for clone AA428416.

SEQ ID NO: 4770 represents the cDNA sequence for clone AA428418.

SEQ ID NO: 4771 represents the cDNA sequence for clone AA429285.

SEQ ID NO: 4772 represents the cDNA sequence for clone AA429286.

SEQ ID NO: 4773 represents the cDNA sequence for clone AA430693.

SEQ ID NO: 4774 represents the cDNA sequence for clone AA427394.

SEQ ID NO: 4775 represents the cDNA sequence for clone AA427521.

SEQ ID NO: 4776 represents the cDNA sequence for clone AA427522.

SEQ ID NO: 4777 represents the cDNA sequence for clone AA428222.

SEQ ID NO: 4778 represents the cDNA sequence for clone AA428224.

SEQ ID NO: 4779 represents the cDNA sequence for clone AA429411.

SEQ ID NO: 4780 represents the cDNA sequence for clone AA429413.

SEQ ID NO: 4781 represents the cDNA sequence for clone AA429414.

SEQ ID NO: 4782 represents the cDNA sequence for clone AA429415.

SEQ ID NO: 4783 represents the cDNA sequence for clone AA429416.

SEQ ID NO: 4784 represents the cDNA sequence for clone AA427538.

SEQ ID NO: 4785 represents the cDNA sequence for clone AA427539.

SEQ ID NO: 4786 represents the cDNA sequence for clone AA427540.

SEQ ID NO: 4787 represents the cDNA sequence for clone AA428351.

SEQ ID NO: 4788 represents the cDNA sequence for clone AA428352.

SEQ ID NO: 4789 represents the cDNA sequence for clone AA429393.

SEQ ID NO: 4790 represents the cDNA sequence for clone AA429394.

SEQ ID NO: 4791 represents the cDNA sequence for clone AA429398.

SEQ ID NO: 4792 represents the cDNA sequence for clone AA429399.

SEQ ID NO: 4793 represents the cDNA sequence for clone AA427780.

SEQ ID NO: 4794 represents the cDNA sequence for clone AA428159.

SEQ ID NO: 4795 represents the cDNA sequence for clone AA430713.

SEQ ID NO: 4796 represents the cDNA sequence for clone AA430718.

SEQ ID NO: 4797 represents the cDNA sequence for clone AA428107.

SEQ ID NO: 4798 represents the cDNA sequence for clone AA428110.

SEQ ID NO: 4799 represents the cDNA sequence for clone AA428111.

SEQ ID NO: 4800 represents the cDNA sequence for clone AA428113.

SEQ ID NO: 4801 represents the cDNA sequence for clone AA427371.

SEQ ID NO: 4802 represents the cDNA sequence for clone AA429042.

SEQ ID NO: 4803 represents the cDNA sequence for clone AA429047.

SEQ ID NO: 4804 represents the cDNA sequence for clone AA429049.

SEQ ID NO: 4805 represents the cDNA sequence for clone AA429427.

SEQ ID NO: 4806 represents the cDNA sequence for clone AA429457.

SEQ ID NO: 4807 represents the cDNA sequence for clone AA429458.

SEQ ID NO: 4808 represents the cDNA sequence for clone AA429462.

SEQ ID NO: 4809 represents the cDNA sequence for clone AA429470.

SEQ ID NO: 4810 represents the cDNA sequence for clone AA429481.

SEQ ID NO: 4811 represents the cDNA sequence for clone AA429484.

SEQ ID NO: 4812 represents the cDNA sequence for clone AA427936.

SEQ ID NO: 4813 represents the cDNA sequence for clone AA428379.

SEQ ID NO: 4814 represents the cDNA sequence for clone AA433808.

SEQ ID NO: 4815 represents the cDNA sequence for clone AA433809.

SEQ ID NO: 4816 represents the cDNA sequence for clone AA433813.

SEQ ID NO: 4817 represents the cDNA sequence for clone AA433850.

SEQ ID NO: 4818 represents the cDNA sequence for clone AA433851.

SEQ ID NO: 4819 represents the cDNA sequence for clone AA433855.

SEQ ID NO: 4820 represents the cDNA sequence for clone AA433858.

SEQ ID NO: 4821 represents the cDNA sequence for clone AA433863.

SEQ ID NO: 4822 represents the cDNA sequence for clone AA433864.

SEQ ID NO: 4823 represents the cDNA sequence for clone AA433865.

SEQ ID NO: 4824 represents the cDNA sequence for clone AA433868.

SEQ ID NO: 4825 represents the cDNA sequence for clone AA433874.

SEQ ID NO: 4826 represents the cDNA sequence for clone AA433882.

SEQ ID NO: 4827 represents the cDNA sequence for clone AA433960.

SEQ ID NO: 4828 represents the cDNA sequence for clone AA433965.

SEQ ID NO: 4829 represents the cDNA sequence for clone AA433967.

SEQ ID NO: 4830 represents the cDNA sequence for clone AA433976.

SEQ ID NO: 4831 represents the cDNA sequence for clone AA433980.

SEQ ID NO: 4832 represents the cDNA sequence for clone AA433988.

SEQ ID NO: 4833 represents the cDNA sequence for clone AA433996.

SEQ ID NO: 4834 represents the cDNA sequence for clone AA433997.

SEQ ID NO: 4835 represents the cDNA sequence for clone AA434004.

SEQ ID NO: 4836 represents the cDNA sequence for clone AA434005.

SEQ ID NO: 4837 represents the cDNA sequence for clone AA434009.

SEQ ID NO: 4838 represents the cDNA sequence for clone AA434010.

SEQ ID NO: 4839 represents the cDNA sequence for clone AA434011.

SEQ ID NO: 4840 represents the cDNA sequence for clone AA434012.

SEQ ID NO: 4841 represents the cDNA sequence for clone AA434013.

SEQ ID NO: 4842 represents the cDNA sequence for clone AA434018.

SEQ ID NO: 4843 represents the cDNA sequence for clone AA434019.

SEQ ID NO: 4844 represents the cDNA sequence for clone AA434020.

SEQ ID NO: 4845 represents the cDNA sequence for clone AA434023.

SEQ ID NO: 4846 represents the cDNA sequence for clone AA434025.

SEQ ID NO: 4847 represents the cDNA sequence for clone AA434029.

SEQ ID NO: 4848 represents the cDNA sequence for clone AA434032.

SEQ ID NO: 4849 represents the cDNA sequence for clone AA434036.

SEQ ID NO: 4850 represents the cDNA sequence for clone AA434041.

SEQ ID NO: 4851 represents the cDNA sequence for clone AA434043.

SEQ ID NO: 4852 represents the cDNA sequence for clone AA434044.

SEQ ID NO: 4853 represents the cDNA sequence for clone AA434047.

SEQ ID NO: 4854 represents the cDNA sequence for clone AA434052.

SEQ ID NO: 4855 represents the cDNA sequence for clone AA434055.

SEQ ID NO: 4856 represents the cDNA sequence for clone AA434056.

SEQ ID NO: 4857 represents the cDNA sequence for clone AA434057.

SEQ ID NO: 4858 represents the cDNA sequence for clone AA434104.

SEQ ID NO: 4859 represents the cDNA sequence for clone AA434109.

SEQ ID NO: 4860 represents the cDNA sequence for clone AA434111.

SEQ ID NO: 4861 represents the cDNA sequence for clone AA434120.

SEQ ID NO: 4862 represents the cDNA sequence for clone AA434121.

SEQ ID NO: 4863 represents the cDNA sequence for clone AA434126.

SEQ ID NO: 4864 represents the cDNA sequence for clone AA434127.

SEQ ID NO: 4865 represents the cDNA sequence for clone AA434130.

SEQ ID NO: 4866 represents the cDNA sequence for clone AA434136.

SEQ ID NO: 4867 represents the cDNA sequence for clone AA434142.

SEQ ID NO: 4868 represents the cDNA sequence for clone AA434145.

SEQ ID NO: 4869 represents the cDNA sequence for clone AA434154.

SEQ ID NO: 4870 represents the cDNA sequence for clone AA434155.

SEQ ID NO: 4871 represents the cDNA sequence for clone AA434156.

SEQ ID NO: 4872 represents the cDNA sequence for clone AA434160.

SEQ ID NO: 4873 represents the cDNA sequence for clone AA434165.

SEQ ID NO: 4874 represents the cDNA sequence for clone AA434166.

SEQ ID NO: 4875 represents the cDNA sequence for clone AA434168.

SEQ ID NO: 4876 represents the cDNA sequence for clone AA434170.

SEQ ID NO: 4877 represents the cDNA sequence for clone AA434172.

SEQ ID NO: 4878 represents the cDNA sequence for clone AA434173.

SEQ ID NO: 4879 represents the cDNA sequence for clone AA434183.

SEQ ID NO: 4880 represents the cDNA sequence for clone AA434185.

SEQ ID NO: 4881 represents the cDNA sequence for clone AA434186.

SEQ ID NO: 4882 represents the cDNA sequence for clone AA434190.

SEQ ID NO: 4883 represents the cDNA sequence for clone AA434192.

SEQ ID NO: 4884 represents the cDNA sequence for clone AA434194.

SEQ ID NO: 4885 represents the cDNA sequence for clone AA434201.

SEQ ID NO: 4886 represents the cDNA sequence for clone AA434213.

SEQ ID NO: 4887 represents the cDNA sequence for clone AA434214.

SEQ ID NO: 4888 represents the cDNA sequence for clone AA434242.

SEQ ID NO: 4889 represents the cDNA sequence for clone AA434243.

SEQ ID NO: 4890 represents the cDNA sequence for clone AA434246.

SEQ ID NO: 4891 represents the cDNA sequence for clone AA434250.

SEQ ID NO: 4892 represents the cDNA sequence for clone AA434252.

SEQ ID NO: 4893 represents the cDNA sequence for clone AA434256.

SEQ ID NO: 4894 represents the cDNA sequence for clone AA434257.

SEQ ID NO: 4895 represents the cDNA sequence for clone AA434261.

SEQ ID NO: 4896 represents the cDNA sequence for clone AA434265.

SEQ ID NO: 4897 represents the cDNA sequence for clone AA434274.

SEQ ID NO: 4898 represents the cDNA sequence for clone AA434280.

SEQ ID NO: 4899 represents the cDNA sequence for clone AA434281.

SEQ ID NO: 4900 represents the cDNA sequence for clone AA434282.

SEQ ID NO: 4901 represents the cDNA sequence for clone AA434288.

SEQ ID NO: 4902 represents the cDNA sequence for clone AA434309.

SEQ ID NO: 4903 represents the cDNA sequence for clone AA434316.

SEQ ID NO: 4904 represents the cDNA sequence for clone AA434326.

SEQ ID NO: 4905 represents the cDNA sequence for clone AA434328.

SEQ ID NO: 4906 represents the cDNA sequence for clone AA434329.

SEQ ID NO: 4907 represents the cDNA sequence for clone AA434330.

SEQ ID NO: 4908 represents the cDNA sequence for clone AA434332.

SEQ ID NO: 4909 represents the cDNA sequence for clone AA434335.

SEQ ID NO: 4910 represents the cDNA sequence for clone AA434388.

SEQ ID NO: 4911 represents the cDNA sequence for clone AA434404.

SEQ ID NO: 4912 represents the cDNA sequence for clone AA434411.

SEQ ID NO: 4913 represents the cDNA sequence for clone AA434419.

SEQ ID NO: 4914 represents the cDNA sequence for clone AA434433.

SEQ ID NO: 4915 represents the cDNA sequence for clone AA434442.

SEQ ID NO: 4916 represents the cDNA sequence for clone AA434446.

SEQ ID NO: 4917 represents the cDNA sequence for clone AA434447.

SEQ ID NO: 4918 represents the cDNA sequence for clone AA434452.

SEQ ID NO: 4919 represents the cDNA sequence for clone AA434484.

SEQ ID NO: 4920 represents the cDNA sequence for clone AA434487.

SEQ ID NO: 4921 represents the cDNA sequence for clone AA434502.

SEQ ID NO: 4922 represents the cDNA sequence for clone AA434508.

SEQ ID NO: 4923 represents the cDNA sequence for clone AA434517.

SEQ ID NO: 4924 represents the cDNA sequence for clone AA434518.

SEQ ID NO: 4925 represents the cDNA sequence for clone AA434520.

SEQ ID NO: 4926 represents the cDNA sequence for clone AA436332.

SEQ ID NO: 4927 represents the cDNA sequence for clone AA436346.

SEQ ID NO: 4928 represents the cDNA sequence for clone AA436350.

SEQ ID NO: 4929 represents the cDNA sequence for clone AA436359.

SEQ ID NO: 4930 represents the cDNA sequence for clone AA436362.

SEQ ID NO: 4931 represents the cDNA sequence for clone AA436366.

SEQ ID NO: 4932 represents the cDNA sequence for clone AA436393.

SEQ ID NO: 4933 represents the cDNA sequence for clone AA436405.

SEQ ID NO: 4934 represents the cDNA sequence for clone AA436409.

SEQ ID NO: 4935 represents the cDNA sequence for clone AA436414.

SEQ ID NO: 4936 represents the cDNA sequence for clone AA436427.

SEQ ID NO: 4937 represents the cDNA sequence for clone AA436432.

SEQ ID NO: 4938 represents the cDNA sequence for clone AA436438.

SEQ ID NO: 4939 represents the cDNA sequence for clone AA436442.

SEQ ID NO: 4940 represents the cDNA sequence for clone AA437329.

SEQ ID NO: 4941 represents the cDNA sequence for clone AA437340.

SEQ ID NO: 4942 represents the cDNA sequence for clone AA437346.

SEQ ID NO: 4943 represents the cDNA sequence for clone AA437349.

SEQ ID NO: 4944 represents the cDNA sequence for clone AA437351.

SEQ ID NO: 4945 represents the cDNA sequence for clone AA437354.

SEQ ID NO: 4946 represents the cDNA sequence for clone AA437356.

SEQ ID NO: 4947 represents the cDNA sequence for clone AA437358.

SEQ ID NO: 4948 represents the cDNA sequence for clone AA437361.

SEQ ID NO: 4949 represents the cDNA sequence for clone AA437369.

SEQ ID NO: 4950 represents the cDNA sequence for clone AA437370.

SEQ ID NO: 4951 represents the cDNA sequence for clone AA437378.

SEQ ID NO: 4952 represents the cDNA sequence for clone AA437379.

SEQ ID NO: 4953 represents the cDNA sequence for clone AA437381.

SEQ ID NO: 4954 represents the cDNA sequence for clone AA437386.

SEQ ID NO: 4955 represents the cDNA sequence for clone AA437388.

SEQ ID NO: 4956 represents the cDNA sequence for clone AA437393.

SEQ ID NO: 4957 represents the cDNA sequence for clone AA442941.

SEQ ID NO: 4958 represents the cDNA sequence for clone AA442944.

SEQ ID NO: 4959 represents the cDNA sequence for clone AA442945.

SEQ ID NO: 4960 represents the cDNA sequence for clone AA442950.

SEQ ID NO: 4961 represents the cDNA sequence for clone AA442953.

SEQ ID NO: 4962 represents the cDNA sequence for clone AA442964.

SEQ ID NO: 4963 represents the cDNA sequence for clone AA442973.

SEQ ID NO: 4964 represents the cDNA sequence for clone AA442977.

SEQ ID NO: 4965 represents the cDNA sequence for clone AA442986.

SEQ ID NO: 4966 represents the cDNA sequence for clone AA442990.

SEQ ID NO: 4967 represents the cDNA sequence for clone AA442994.

SEQ ID NO: 4968 represents the cDNA sequence for clone AA443087.

SEQ ID NO: 4969 represents the cDNA sequence for clone AA443093.

SEQ ID NO: 4970 represents the cDNA sequence for clone AA443102.

SEQ ID NO: 4971 represents the cDNA sequence for clone AA443115.

SEQ ID NO: 4972 represents the cDNA sequence for clone AA443117.

SEQ ID NO: 4973 represents the cDNA sequence for clone AA443261.

SEQ ID NO: 4974 represents the cDNA sequence for clone AA443262.

SEQ ID NO: 4975 represents the cDNA sequence for clone AA443263.

SEQ ID NO: 4976 represents the cDNA sequence for clone AA443265.

SEQ ID NO: 4977 represents the cDNA sequence for clone AA443267.

SEQ ID NO: 4978 represents the cDNA sequence for clone AA443471.

SEQ ID NO: 4979 represents the cDNA sequence for clone AA443473.

SEQ ID NO: 4980 represents the cDNA sequence for clone AA443475.

SEQ ID NO: 4981 represents the cDNA sequence for clone AA443479.

SEQ ID NO: 4982 represents the cDNA sequence for clone AA443480.

SEQ ID NO: 4983 represents the cDNA sequence for clone AA443487.

SEQ ID NO: 4984 represents the cDNA sequence for clone AA443489.

SEQ ID NO: 4985 represents the cDNA sequence for clone AA443498.

SEQ ID NO: 4986 represents the cDNA sequence for clone AA443507.

SEQ ID NO: 4987 represents the cDNA sequence for clone AA443512.

SEQ ID NO: 4988 represents the cDNA sequence for clone AA443514.

SEQ ID NO: 4989 represents the cDNA sequence for clone AA443519.

SEQ ID NO: 4990 represents the cDNA sequence for clone AA443523.

SEQ ID NO: 4991 represents the cDNA sequence for clone AA443528.

SEQ ID NO: 4992 represents the cDNA sequence for clone AA443532.

SEQ ID NO: 4993 represents the cDNA sequence for clone AA443561.

SEQ ID NO: 4994 represents the cDNA sequence for clone AA443562.

SEQ ID NO: 4995 represents the cDNA sequence for clone AA443566.

SEQ ID NO: 4996 represents the cDNA sequence for clone AA443567.

SEQ ID NO: 4997 represents the cDNA sequence for clone AA443569.

SEQ ID NO: 4998 represents the cDNA sequence for clone AA443573.

SEQ ID NO: 4999 represents the cDNA sequence for clone AA443575.

SEQ ID NO: 5000 represents the cDNA sequence for clone AA443581.

SEQ ID NO: 5001 represents the cDNA sequence for clone AA443584.

SEQ ID NO: 5002 represents the cDNA sequence for clone AA443587.

SEQ ID NO: 5003 represents the cDNA sequence for clone AA443594.

SEQ ID NO: 5004 represents the cDNA sequence for clone AA443605.

SEQ ID NO: 5005 represents the cDNA sequence for clone AA443608.

SEQ ID NO: 5006 represents the cDNA sequence for clone AA443615.

SEQ ID NO: 5007 represents the cDNA sequence for clone AA443618.

SEQ ID NO: 5008 represents the cDNA sequence for clone AA443622.

SEQ ID NO: 5009 represents the cDNA sequence for clone AA443630.

SEQ ID NO: 5010 represents the cDNA sequence for clone AA443635.

SEQ ID NO: 5011 represents the cDNA sequence for clone AA443636.

SEQ ID NO: 5012 represents the cDNA sequence for clone AA443642.

SEQ ID NO: 5013 represents the cDNA sequence for clone AA443644.

SEQ ID NO: 5014 represents the cDNA sequence for clone AA443646.

SEQ ID NO: 5015 represents the cDNA sequence for clone AA443649.

SEQ ID NO: 5016 represents the cDNA sequence for clone AA443884.

SEQ ID NO: 5017 represents the cDNA sequence for clone AA443888.

SEQ ID NO: 5018 represents the cDNA sequence for clone AA443891.

SEQ ID NO: 5019 represents the cDNA sequence for clone AA443898.

SEQ ID NO: 5020 represents the cDNA sequence for clone AA443904.

SEQ ID NO: 5021 represents the cDNA sequence for clone AA443917.

SEQ ID NO: 5022 represents the cDNA sequence for clone AA443919.

SEQ ID NO: 5023 represents the cDNA sequence for clone AA443921.

SEQ ID NO: 5024 represents the cDNA sequence for clone AA443991.

SEQ ID NO: 5025 represents the cDNA sequence for clone AA443994.

SEQ ID NO: 5026 represents the cDNA sequence for clone AA443995.

SEQ ID NO: 5027 represents the cDNA sequence for clone AA443996.

SEQ ID NO: 5028 represents the cDNA sequence for clone AA443998.

SEQ ID NO: 5029 represents the cDNA sequence for clone AA444010.

SEQ ID NO: 5030 represents the cDNA sequence for clone AA444012.

SEQ ID NO: 5031 represents the cDNA sequence for clone AA444016.

SEQ ID NO: 5032 represents the cDNA sequence for clone AA444017.

SEQ ID NO: 5033 represents the cDNA sequence for clone AA444018.

SEQ ID NO: 5034 represents the cDNA sequence for clone AA444020.

SEQ ID NO: 5035 represents the cDNA sequence for clone AA444049.

SEQ ID NO: 5036 represents the cDNA sequence for clone AA444052.

SEQ ID NO: 5037 represents the cDNA sequence for clone AA444062.

SEQ ID NO: 5038 represents the cDNA sequence for clone AA444066.

SEQ ID NO: 5039 represents the cDNA sequence for clone AA444068.

SEQ ID NO: 5040 represents the cDNA sequence for clone AA444071.

SEQ ID NO: 5041 represents the cDNA sequence for clone AA444077.

SEQ ID NO: 5042 represents the cDNA sequence for clone AA444080.

SEQ ID NO: 5043 represents the cDNA sequence for clone AA444083.

SEQ ID NO: 5044 represents the cDNA sequence for clone AA444092.

SEQ ID NO: 5045 represents the cDNA sequence for clone AA444100.

SEQ ID NO: 5046 represents the cDNA sequence for clone AA453113.

SEQ ID NO: 5047 represents the cDNA sequence for clone AA456762.

SEQ ID NO: 5048 represents the cDNA sequence for clone AA456766.

SEQ ID NO: 5049 represents the cDNA sequence for clone AA456767.

SEQ ID NO: 5050 represents the cDNA sequence for clone AA456769.

SEQ ID NO: 5051 represents the cDNA sequence for clone AA456777.

SEQ ID NO: 5052 represents the cDNA sequence for clone AA456780.

SEQ ID NO: 5053 represents the cDNA sequence for clone AA456785.

SEQ ID NO: 5054 represents the cDNA sequence for clone AA456787.

SEQ ID NO: 5055 represents the cDNA sequence for clone AA456793.

SEQ ID NO: 5056 represents the cDNA sequence for clone AA456800.

SEQ ID NO: 5057 represents the cDNA sequence for clone AA456808.

SEQ ID NO: 5058 represents the cDNA sequence for clone AA456810.

SEQ ID NO: 5059 represents the cDNA sequence for clone AA454462.

SEQ ID NO: 5060 represents the cDNA sequence for clone AA454467.

SEQ ID NO: 5061 represents the cDNA sequence for clone AA454469.

SEQ ID NO: 5062 represents the cDNA sequence for clone AA454488.

SEQ ID NO: 5063 represents the cDNA sequence for clone AA454492.

SEQ ID NO: 5064 represents the cDNA sequence for clone AA454495.

SEQ ID NO: 5065 represents the cDNA sequence for clone AA454496.

SEQ ID NO: 5066 represents the cDNA sequence for clone AA454497.

SEQ ID NO: 5067 represents the cDNA sequence for clone AA454500.

SEQ ID NO: 5068 represents the cDNA sequence for clone AA454503.

SEQ ID NO: 5069 represents the cDNA sequence for clone AA454506.

SEQ ID NO: 5070 represents the cDNA sequence for clone AA454517.

SEQ ID NO: 5071 represents the cDNA sequence for clone AA454518.

SEQ ID NO: 5072 represents the cDNA sequence for clone AA454520.

SEQ ID NO: 5073 represents the cDNA sequence for clone AA454521.

SEQ ID NO: 5074 represents the cDNA sequence for clone AA454522.

SEQ ID NO: 5075 represents the cDNA sequence for clone AA454549.

SEQ ID NO: 5076 represents the cDNA sequence for clone AA454552.

SEQ ID NO: 5077 represents the cDNA sequence for clone AA454554.

SEQ ID NO: 5078 represents the cDNA sequence for clone AA454562.

SEQ ID NO: 5079 represents the cDNA sequence for clone AA454570.

SEQ ID NO: 5080 represents the cDNA sequence for clone AA454571.

SEQ ID NO: 5081 represents the cDNA sequence for clone AA454586.

SEQ ID NO: 5082 represents the cDNA sequence for clone AA454595.

SEQ ID NO: 5083 represents the cDNA sequence for clone AA454605.

SEQ ID NO: 5084 represents the cDNA sequence for clone AA454607.

SEQ ID NO: 5085 represents the cDNA sequence for clone AA454609.

SEQ ID NO: 5086 represents the cDNA sequence for clone AA454610.

SEQ ID NO: 5087 represents the cDNA sequence for clone AA454612.

SEQ ID NO: 5088 represents the cDNA sequence for clone AA454616.

SEQ ID NO: 5089 represents the cDNA sequence for clone AA454674.

SEQ ID NO: 5090 represents the cDNA sequence for clone AA454675.

SEQ ID NO: 5091 represents the cDNA sequence for clone AA454685.

SEQ ID NO: 5092 represents the cDNA sequence for clone AA454689.

SEQ ID NO: 5093 represents the cDNA sequence for clone AA454690.

SEQ ID NO: 5094 represents the cDNA sequence for clone AA454696.

SEQ ID NO: 5095 represents the cDNA sequence for clone AA454697.

SEQ ID NO: 5096 represents the cDNA sequence for clone AA454704.

SEQ ID NO: 5097 represents the cDNA sequence for clone AA454711.

SEQ ID NO: 5098 represents the cDNA sequence for clone AA454723.

SEQ ID NO: 5099 represents the cDNA sequence for clone AA454724.

SEQ ID NO: 5100 represents the cDNA sequence for clone AA454725.

SEQ ID NO: 5101 represents the cDNA sequence for clone AA454728.

SEQ ID NO: 5102 represents the cDNA sequence for clone AA454731.

SEQ ID NO: 5103 represents the cDNA sequence for clone AA454732.

SEQ ID NO: 5104 represents the cDNA sequence for clone AA454747.

SEQ ID NO: 5105 represents the cDNA sequence for clone AA454751.

SEQ ID NO: 5106 represents the cDNA sequence for clone AA454753.

SEQ ID NO: 5107 represents the cDNA sequence for clone AA454755.

SEQ ID NO: 5108 represents the cDNA sequence for clone AA454756.

SEQ ID NO: 5109 represents the cDNA sequence for clone AA454758.

SEQ ID NO: 5110 represents the cDNA sequence for clone AA454760.

SEQ ID NO: 5111 represents the cDNA sequence for clone AA454773.

SEQ ID NO: 5112 represents the cDNA sequence for clone AA454774.

SEQ ID NO: 5113 represents the cDNA sequence for clone AA454775.

SEQ ID NO: 5114 represents the cDNA sequence for clone AA454776.

SEQ ID NO: 5115 represents the cDNA sequence for clone AA454777.

SEQ ID NO: 5116 represents the cDNA sequence for clone AA454779.

SEQ ID NO: 5117 represents the cDNA sequence for clone AA454784.

SEQ ID NO: 5118 represents the cDNA sequence for clone AA454785.

SEQ ID NO: 5119 represents the cDNA sequence for clone AA454797.

SEQ ID NO: 5120 represents the cDNA sequence for clone AA454801.

SEQ ID NO: 5121 represents the cDNA sequence for clone AA454802.

SEQ ID NO: 5122 represents the cDNA sequence for clone AA454803.

SEQ ID NO: 5123 represents the cDNA sequence for clone AA454823.

SEQ ID NO: 5124 represents the cDNA sequence for clone AA454824.

SEQ ID NO: 5125 represents the cDNA sequence for clone AA454827.

SEQ ID NO: 5126 represents the cDNA sequence for clone AA454832.

SEQ ID NO: 5127 represents the cDNA sequence for clone AA454835.

SEQ ID NO: 5128 represents the cDNA sequence for clone AA454837.

SEQ ID NO: 5129 represents the cDNA sequence for clone AA454839.

SEQ ID NO: 5130 represents the cDNA sequence for clone AA454844.

SEQ ID NO: 5131 represents the cDNA sequence for clone AA454861.

SEQ ID NO: 5132 represents the cDNA sequence for clone AA454871.

SEQ ID NO: 5133 represents the cDNA sequence for clone AA454875.

SEQ ID NO: 5134 represents the cDNA sequence for clone AA454878.

SEQ ID NO: 5135 represents the cDNA sequence for clone AA454881.

SEQ ID NO: 5136 represents the cDNA sequence for clone AA454887.

SEQ ID NO: 5137 represents the cDNA sequence for clone AA454896.

SEQ ID NO: 5138 represents the cDNA sequence for clone AA454898.

SEQ ID NO: 5139 represents the cDNA sequence for clone AA454899.

SEQ ID NO: 5140 represents the cDNA sequence for clone AA454902.

SEQ ID NO: 5141 represents the cDNA sequence for clone AA454905.

SEQ ID NO: 5142 represents the cDNA sequence for clone AA454907.

SEQ ID NO: 5143 represents the cDNA sequence for clone AA454911.

SEQ ID NO: 5144 represents the cDNA sequence for clone AA454912.

SEQ ID NO: 5145 represents the cDNA sequence for clone AA454913.

SEQ ID NO: 5146 represents the cDNA sequence for clone AA455018.

SEQ ID NO: 5147 represents the cDNA sequence for clone AA455022.

SEQ ID NO: 5148 represents the cDNA sequence for clone AA455026.

SEQ ID NO: 5149 represents the cDNA sequence for clone AA455036.

SEQ ID NO: 5150 represents the cDNA sequence for clone AA455103.

SEQ ID NO: 5151 represents the cDNA sequence for clone AA455106.

SEQ ID NO: 5152 represents the cDNA sequence for clone AA455111.

SEQ ID NO: 5153 represents the cDNA sequence for clone AA455116.

SEQ ID NO: 5154 represents the cDNA sequence for clone AA455130.

SEQ ID NO: 5155 represents the cDNA sequence for clone AA455131.

SEQ ID NO: 5156 represents the cDNA sequence for clone AA455133.

SEQ ID NO: 5157 represents the cDNA sequence for clone AA455139.

SEQ ID NO: 5158 represents the cDNA sequence for clone AA455150.

SEQ ID NO: 5159 represents the cDNA sequence for clone AA455157.

SEQ ID NO: 5160 represents the cDNA sequence for clone AA455185.

SEQ ID NO: 5161 represents the cDNA sequence for clone AA455187.

SEQ ID NO: 5162 represents the cDNA sequence for clone AA455192.

SEQ ID NO: 5163 represents the cDNA sequence for clone AA455196.

SEQ ID NO: 5164 represents the cDNA sequence for clone AA455212.

SEQ ID NO: 5165 represents the cDNA sequence for clone AA455218.

SEQ ID NO: 5166 represents the cDNA sequence for clone AA455272.

SEQ ID NO: 5167 represents the cDNA sequence for clone AA455274.

SEQ ID NO: 5168 represents the cDNA sequence for clone AA455279.

SEQ ID NO: 5169 represents the cDNA sequence for clone AA455282.

SEQ ID NO: 5170 represents the cDNA sequence for clone AA455287.

SEQ ID NO: 5171 represents the cDNA sequence for clone AA455288.

SEQ ID NO: 5172 represents the cDNA sequence for clone AA455292.

SEQ ID NO: 5173 represents the cDNA sequence for clone AA455294.

SEQ ID NO: 5174 represents the cDNA sequence for clone AA455295.

SEQ ID NO: 5175 represents the cDNA sequence for clone AA455297.

SEQ ID NO: 5176 represents the cDNA sequence for clone AA455302.

SEQ ID NO: 5177 represents the cDNA sequence for clone AA455304.

SEQ ID NO: 5178 represents the cDNA sequence for clone AA455305.

SEQ ID NO: 5179 represents the cDNA sequence for clone AA455484.

SEQ ID NO: 5180 represents the cDNA sequence for clone AA455485.

SEQ ID NO: 5181 represents the cDNA sequence for clone AA455492.

SEQ ID NO: 5182 represents the cDNA sequence for clone AA455493.

SEQ ID NO: 5183 represents the cDNA sequence for clone AA455502.

SEQ ID NO: 5184 represents the cDNA sequence for clone AA455503.

SEQ ID NO: 5185 represents the cDNA sequence for clone AA455508.

SEQ ID NO: 5186 represents the cDNA sequence for clone AA455511.

SEQ ID NO: 5187 represents the cDNA sequence for clone AA455515.

SEQ ID NO: 5188 represents the cDNA sequence for clone AA455520.

SEQ ID NO: 5189 represents the cDNA sequence for clone AA455521.

SEQ ID NO: 5190 represents the cDNA sequence for clone AA455524.

SEQ ID NO: 5191 represents the cDNA sequence for clone AA455767.

SEQ ID NO: 5192 represents the cDNA sequence for clone AA455776.

SEQ ID NO: 5193 represents the cDNA sequence for clone AA455797.

SEQ ID NO: 5194 represents the cDNA sequence for clone AA455809.

SEQ ID NO: 5195 represents the cDNA sequence for clone AA455811.

SEQ ID NO: 5196 represents the cDNA sequence for clone AA455812.

SEQ ID NO: 5197 represents the cDNA sequence for clone AA455823.

SEQ ID NO: 5198 represents the cDNA sequence for clone AA455828.

SEQ ID NO: 5199 represents the cDNA sequence for clone AA455830.

SEQ ID NO: 5200 represents the cDNA sequence for clone AA455833.

SEQ ID NO: 5201 represents the cDNA sequence for clone AA455839.

SEQ ID NO: 5202 represents the cDNA sequence for clone AA455844.

SEQ ID NO: 5203 represents the cDNA sequence for clone AA455851.

SEQ ID NO: 5204 represents the cDNA sequence for clone AA455852.

SEQ ID NO: 5205 represents the cDNA sequence for clone AA455855.

SEQ ID NO: 5206 represents the cDNA sequence for clone AA456118.

SEQ ID NO: 5207 represents the cDNA sequence for clone AA456120.

SEQ ID NO: 5208 represents the cDNA sequence for clone AA456121.

SEQ ID NO: 5209 represents the cDNA sequence for clone AA456329.

SEQ ID NO: 5210 represents the cDNA sequence for clone AA456330.

SEQ ID NO: 5211 represents the cDNA sequence for clone AA456334.

SEQ ID NO: 5212 represents the cDNA sequence for clone AA456344.

SEQ ID NO: 5213 represents the cDNA sequence for clone AA456346.

SEQ ID NO: 5214 represents the cDNA sequence for clone AA456349.

SEQ ID NO: 5215 represents the cDNA sequence for clone AA456350.

SEQ ID NO: 5216 represents the cDNA sequence for clone AA456357.

SEQ ID NO: 5217 represents the cDNA sequence for clone AA456359.

SEQ ID NO: 5218 represents the cDNA sequence for clone AA456363.

SEQ ID NO: 5219 represents the cDNA sequence for clone AA456441.

SEQ ID NO: 5220 represents the cDNA sequence for clone AA456442.

SEQ ID NO: 5221 represents the cDNA sequence for clone AA456444.

SEQ ID NO: 5222 represents the cDNA sequence for clone AA456446.

SEQ ID NO: 5223 represents the cDNA sequence for clone AA456454.

SEQ ID NO: 5224 represents the cDNA sequence for clone AA456455.

SEQ ID NO: 5225 represents the cDNA sequence for clone AA456456.

SEQ ID NO: 5226 represents the cDNA sequence for clone AA456458.

SEQ ID NO: 5227 represents the cDNA sequence for clone AA456461.

SEQ ID NO: 5228 represents the cDNA sequence for clone AA456464.

SEQ ID NO: 5229 represents the cDNA sequence for clone AA456465.

SEQ ID NO: 5230 represents the cDNA sequence for clone AA456472.

SEQ ID NO: 5231 represents the cDNA sequence for clone AA456477.

SEQ ID NO: 5232 represents the cDNA sequence for clone AA456480.

SEQ ID NO: 5233 represents the cDNA sequence for clone AA456566.

SEQ ID NO: 5234 represents the cDNA sequence for clone AA456592.

SEQ ID NO: 5235 represents the cDNA sequence for clone AA456594.

SEQ ID NO: 5236 represents the cDNA sequence for clone AA456599.

SEQ ID NO: 5237 represents the cDNA sequence for clone AA456618.

SEQ ID NO: 5238 represents the cDNA sequence for clone AA456623.

SEQ ID NO: 5239 represents the cDNA sequence for clone AA456628.

SEQ ID NO: 5240 represents the cDNA sequence for clone AA456630.

SEQ ID NO: 5241 represents the cDNA sequence for clone AA456154.

SEQ ID NO: 5242 represents the cDNA sequence for clone AA456157.

SEQ ID NO: 5243 represents the cDNA sequence for clone AA456160.

SEQ ID NO: 5244 represents the cDNA sequence for clone AA456166.

SEQ ID NO: 5245 represents the cDNA sequence for clone AA456171.

SEQ ID NO: 5246 represents the cDNA sequence for clone AA456180.

SEQ ID NO: 5247 represents the cDNA sequence for clone AA456181.

SEQ ID NO: 5248 represents the cDNA sequence for clone AA456186.

SEQ ID NO: 5249 represents the cDNA sequence for clone AA457109.

SEQ ID NO: 5250 represents the cDNA sequence for clone AA457117.

SEQ ID NO: 5251 represents the cDNA sequence for clone AA457130.

SEQ ID NO: 5252 represents the cDNA sequence for clone AA457141.

SEQ ID NO: 5253 represents the cDNA sequence for clone AA457148.

SEQ ID NO: 5254 represents the cDNA sequence for clone AA457154.

SEQ ID NO: 5255 represents the cDNA sequence for clone AA457159.

SEQ ID NO: 5256 represents the cDNA sequence for clone AA457686.

SEQ ID NO: 5257 represents the cDNA sequence for clone AA457688.

SEQ ID NO: 5258 represents the cDNA sequence for clone AA457706.

SEQ ID NO: 5259 represents the cDNA sequence for clone AA457708.

SEQ ID NO: 5260 represents the cDNA sequence for clone AA457716.

SEQ ID NO: 5261 represents the cDNA sequence for clone AA457729.

SEQ ID NO: 5262 represents the cDNA sequence for clone AA459895.

SEQ ID NO: 5263 represents the cDNA sequence for clone AA459901.

SEQ ID NO: 5264 represents the cDNA sequence for clone AA459906.

SEQ ID NO: 5265 represents the cDNA sequence for clone AA459912.

SEQ ID NO: 5266 represents the cDNA sequence for clone AA458453.

SEQ ID NO: 5267 represents the cDNA sequence for clone AA458454.

SEQ ID NO: 5268 represents the cDNA sequence for clone AA458460.

SEQ ID NO: 5269 represents the cDNA sequence for clone AA458464.

SEQ ID NO: 5270 represents the cDNA sequence for clone AA458473.

SEQ ID NO: 5271 represents the cDNA sequence for clone AA458479.

SEQ ID NO: 5272 represents the cDNA sequence for clone AA458484.

SEQ ID NO: 5273 represents the cDNA sequence for clone AA458485.

SEQ ID NO: 5274 represents the cDNA sequence for clone AA458488.

SEQ ID NO: 5275 represents the cDNA sequence for clone AA458489.

SEQ ID NO: 5276 represents the cDNA sequence for clone AA458491.

SEQ ID NO: 5277 represents the cDNA sequence for clone AA458498.

SEQ ID NO: 5278 represents the cDNA sequence for clone AA458499.

SEQ ID NO: 5279 represents the cDNA sequence for clone AA458503.

SEQ ID NO: 5280 represents the cDNA sequence for clone AA458509.

SEQ ID NO: 5281 represents the cDNA sequence for clone AA458513.

SEQ ID NO: 5282 represents the cDNA sequence for clone AA458518.

SEQ ID NO: 5283 represents the cDNA sequence for clone AA458520.

SEQ ID NO: 5284 represents the cDNA sequence for clone AA458524.

SEQ ID NO: 5285 represents the cDNA sequence for clone AA458533.

SEQ ID NO: 5286 represents the cDNA sequence for clone AA458534.

SEQ ID NO: 5287 represents the cDNA sequence for clone AA458536.

SEQ ID NO: 5288 represents the cDNA sequence for clone AA458537.

SEQ ID NO: 5289 represents the cDNA sequence for clone AA458540.

SEQ ID NO: 5290 represents the cDNA sequence for clone AA458871.

SEQ ID NO: 5291 represents the cDNA sequence for clone AA458873.

SEQ ID NO: 5292 represents the cDNA sequence for clone AA458876.

SEQ ID NO: 5293 represents the cDNA sequence for clone AA458884.

SEQ ID NO: 5294 represents the cDNA sequence for clone AA458886.

SEQ ID NO: 5295 represents the cDNA sequence for clone AA458889.

SEQ ID NO: 5296 represents the cDNA sequence for clone AA458893.

SEQ ID NO: 5297 represents the cDNA sequence for clone AA458895.

SEQ ID NO: 5298 represents the cDNA sequence for clone AA458897.

SEQ ID NO: 5299 represents the cDNA sequence for clone AA458902.

SEQ ID NO: 5300 represents the cDNA sequence for clone AA458980.

SEQ ID NO: 5301 represents the cDNA sequence for clone AA458983.

SEQ ID NO: 5302 represents the cDNA sequence for clone AA459059.

SEQ ID NO: 5303 represents the cDNA sequence for clone AA459060.

SEQ ID NO: 5304 represents the cDNA sequence for clone AA459063.

SEQ ID NO: 5305 represents the cDNA sequence for clone AA459064.

SEQ ID NO: 5306 represents the cDNA sequence for clone AA459066.

SEQ ID NO: 5307 represents the cDNA sequence for clone AA459069.

SEQ ID NO: 5308 represents the cDNA sequence for clone AA459074.

SEQ ID NO: 5309 represents the cDNA sequence for clone AA459075.

SEQ ID NO: 5310 represents the cDNA sequence for clone AA459076.

SEQ ID NO: 5311 represents the cDNA sequence for clone AA459077.

SEQ ID NO: 5312 represents the cDNA sequence for clone AA459085.

SEQ ID NO: 5313 represents the cDNA sequence for clone AA459090.

SEQ ID NO: 5314 represents the cDNA sequence for clone AA459092.

SEQ ID NO: 5315 represents the cDNA sequence for clone AA459169.

SEQ ID NO: 5316 represents the cDNA sequence for clone AA459182.

SEQ ID NO: 5317 represents the cDNA sequence for clone AA459191.

SEQ ID NO: 5318 represents the cDNA sequence for clone AA459198.

SEQ ID NO: 5319 represents the cDNA sequence for clone AA459199.

SEQ ID NO: 5320 represents the cDNA sequence for clone AA459201.

SEQ ID NO: 5321 represents the cDNA sequence for clone AA459277.

SEQ ID NO: 5322 represents the cDNA sequence for clone AA459279.

SEQ ID NO: 5323 represents the cDNA sequence for clone AA459280.

SEQ ID NO: 5324 represents the cDNA sequence for clone AA459281.

SEQ ID NO: 5325 represents the cDNA sequence for clone AA459287.

SEQ ID NO: 5326 represents the cDNA sequence for clone AA459379.

SEQ ID NO: 5327 represents the cDNA sequence for clone AA459384.

SEQ ID NO: 5328 represents the cDNA sequence for clone AA459386.

SEQ ID NO: 5329 represents the cDNA sequence for clone AA459392.

SEQ ID NO: 5330 represents the cDNA sequence for clone AA459401.

SEQ ID NO: 5331 represents the cDNA sequence for clone AA459405.

SEQ ID NO: 5332 represents the cDNA sequence for clone AA459407.

SEQ ID NO: 5333 represents the cDNA sequence for clone AA459410.

SEQ ID NO: 5334 represents the cDNA sequence for clone AA459421.

SEQ ID NO: 5335 represents the cDNA sequence for clone AA459506.

SEQ ID NO: 5336 represents the cDNA sequence for clone AA459508.

SEQ ID NO: 5337 represents the cDNA sequence for clone AA459510.

SEQ ID NO: 5338 represents the cDNA sequence for clone AA459511.

SEQ ID NO: 5339 represents the cDNA sequence for clone AA459516.

SEQ ID NO: 5340 represents the cDNA sequence for clone AA459518.

SEQ ID NO: 5341 represents the cDNA sequence for clone AA459519.

SEQ ID NO: 5342 represents the cDNA sequence for clone AA459523.

SEQ ID NO: 5343 represents the cDNA sequence for clone AA459525.

SEQ ID NO: 5344 represents the cDNA sequence for clone AA459531.

SEQ ID NO: 5345 represents the cDNA sequence for clone AA459536.

SEQ ID NO: 5346 represents the cDNA sequence for clone AA459605.

SEQ ID NO: 5347 represents the cDNA sequence for clone AA459609.

SEQ ID NO: 5348 represents the cDNA sequence for clone AA459611.

SEQ ID NO: 5349 represents the cDNA sequence for clone AA459612.

SEQ ID NO: 5350 represents the cDNA sequence for clone AA459614.

SEQ ID NO: 5351 represents the cDNA sequence for clone AA459617.

SEQ ID NO: 5352 represents the cDNA sequence for clone AA459626.

SEQ ID NO: 5353 represents the cDNA sequence for clone AA459632.

SEQ ID NO: 5354 represents the cDNA sequence for clone AA459635.

SEQ ID NO: 5355 represents the cDNA sequence for clone AA459645.

SEQ ID NO: 5356 represents the cDNA sequence for clone AA463928.

SEQ ID NO: 5357 represents the cDNA sequence for clone AA463944.

SEQ ID NO: 5358 represents the cDNA sequence for clone AA463948.

SEQ ID NO: 5359 represents the cDNA sequence for clone AA463950.

SEQ ID NO: 5360 represents the cDNA sequence for clone AA463955.

SEQ ID NO: 5361 represents the cDNA sequence for clone AA463960.

SEQ ID NO: 5362 represents the cDNA sequence for clone AA463965.

SEQ ID NO: 5363 represents the cDNA sequence for clone AA463974.

SEQ ID NO: 5364 represents the cDNA sequence for clone AA463975.

SEQ ID NO: 5365 represents the cDNA sequence for clone AA463988.

SEQ ID NO: 5366 represents the cDNA sequence for clone AA463990.

SEQ ID NO: 5367 represents the cDNA sequence for clone AA463995.

SEQ ID NO: 5368 represents the cDNA sequence for clone AA463996.

SEQ ID NO: 5369 represents the cDNA sequence for clone AA463999.

SEQ ID NO: 5370 represents the cDNA sequence for clone AA464002.

SEQ ID NO: 5371 represents the cDNA sequence for clone AA464004.

SEQ ID NO: 5372 represents the cDNA sequence for clone AA464011.

SEQ ID NO: 5373 represents the cDNA sequence for clone AA464018.

SEQ ID NO: 5374 represents the cDNA sequence for clone AA464021.

SEQ ID NO: 5375 represents the cDNA sequence for clone AA464030.

SEQ ID NO: 5376 represents the cDNA sequence for clone AA464031.

SEQ ID NO: 5377 represents the cDNA sequence for clone AA464033.

SEQ ID NO: 5378 represents the cDNA sequence for clone AA464036.

SEQ ID NO: 5379 represents the cDNA sequence for clone AA464038.

SEQ ID NO: 5380 represents the cDNA sequence for clone AA464040.

SEQ ID NO: 5381 represents the cDNA sequence for clone AA464044.

SEQ ID NO: 5382 represents the cDNA sequence for clone AA464052.

SEQ ID NO: 5383 represents the cDNA sequence for clone AA464055.

SEQ ID NO: 5384 represents the cDNA sequence for clone AA464056.

SEQ ID NO: 5385 represents the cDNA sequence for clone AA464064.

SEQ ID NO: 5386 represents the cDNA sequence for clone AA464068.

SEQ ID NO: 5387 represents the cDNA sequence for clone AA464072.

SEQ ID NO: 5388 represents the cDNA sequence for clone AA464079.

SEQ ID NO: 5389 represents the cDNA sequence for clone AA464091.

SEQ ID NO: 5390 represents the cDNA sequence for clone AA464097.

SEQ ID NO: 5391 represents the cDNA sequence for clone AA464102.

SEQ ID NO: 5392 represents the cDNA sequence for clone AA464110.

SEQ ID NO: 5393 represents the cDNA sequence for clone AA464111.

SEQ ID NO: 5394 represents the cDNA sequence for clone AA464124.

SEQ ID NO: 5395 represents the cDNA sequence for clone AA464138.

SEQ ID NO: 5396 represents the cDNA sequence for clone AA464140.

SEQ ID NO: 5397 represents the cDNA sequence for clone AA464141.

SEQ ID NO: 5398 represents the cDNA sequence for clone AA464144.

SEQ ID NO: 5399 represents the cDNA sequence for clone AA464146.

SEQ ID NO: 5400 represents the cDNA sequence for clone AA464156.

SEQ ID NO: 5401 represents the cDNA sequence for clone AA464162.

SEQ ID NO: 5402 represents the cDNA sequence for clone AA464166.

SEQ ID NO: 5403 represents the cDNA sequence for clone AA464173.

SEQ ID NO: 5404 represents the cDNA sequence for clone AA464176.

SEQ ID NO: 5405 represents the cDNA sequence for clone AA464181.

SEQ ID NO: 5406 represents the cDNA sequence for clone AA464191.

SEQ ID NO: 5407 represents the cDNA sequence for clone AA464200.

SEQ ID NO: 5408 represents the cDNA sequence for clone AA464204.

SEQ ID NO: 5409 represents the cDNA sequence for clone AA464205.

SEQ ID NO: 5410 represents the cDNA sequence for clone AA464207.

SEQ ID NO: 5411 represents the cDNA sequence for clone AA464208.

SEQ ID NO: 5412 represents the cDNA sequence for clone AA464210.

SEQ ID NO: 5413 represents the cDNA sequence for clone AA464212.

SEQ ID NO: 5414 represents the cDNA sequence for clone AA464216.

SEQ ID NO: 5415 represents the cDNA sequence for clone AA464222.

SEQ ID NO: 5416 represents the cDNA sequence for clone AA464239.

SEQ ID NO: 5417 represents the cDNA sequence for clone AA464243.

SEQ ID NO: 5418 represents the cDNA sequence for clone AA464254.

SEQ ID NO: 5419 represents the cDNA sequence for clone AA464255.

SEQ ID NO: 5420 represents the cDNA sequence for clone AA464266.

SEQ ID NO: 5421 represents the cDNA sequence for clone AA464271.

SEQ ID NO: 5422 represents the cDNA sequence for clone AA464273.

SEQ ID NO: 5423 represents the cDNA sequence for clone AA464279.

SEQ ID NO: 5424 represents the cDNA sequence for clone AA464285.

SEQ ID NO: 5425 represents the cDNA sequence for clone AA464287.

SEQ ID NO: 5426 represents the cDNA sequence for clone AA464290.

SEQ ID NO: 5427 represents the cDNA sequence for clone AA464295.

SEQ ID NO: 5428 represents the cDNA sequence for clone AA464301.

SEQ ID NO: 5429 represents the cDNA sequence for clone AA464305.

SEQ ID NO: 5430 represents the cDNA sequence for clone AA464307.

SEQ ID NO: 5431 represents the cDNA sequence for clone AA464308.

SEQ ID NO: 5432 represents the cDNA sequence for clone AA464326.

SEQ ID NO: 5433 represents the cDNA sequence for clone AA464335.

SEQ ID NO: 5434 represents the cDNA sequence for clone AA464343.

SEQ ID NO: 5435 represents the cDNA sequence for clone AA464347.

SEQ ID NO: 5436 represents the cDNA sequence for clone AA464348.

SEQ ID NO: 5437 represents the cDNA sequence for clone AA464351.

SEQ ID NO: 5438 represents the cDNA sequence for clone AA464359.

SEQ ID NO: 5439 represents the cDNA sequence for clone AA464362.

SEQ ID NO: 5440 represents the cDNA sequence for clone AA464365.

SEQ ID NO: 5441 represents the cDNA sequence for clone AA464366.

SEQ ID NO: 5442 represents the cDNA sequence for clone AA464369.

SEQ ID NO: 5443 represents the cDNA sequence for clone AA464375.

SEQ ID NO: 5444 represents the cDNA sequence for clone AA464379.

SEQ ID NO: 5445 represents the cDNA sequence for clone AA464380.

SEQ ID NO: 5446 represents the cDNA sequence for clone AA464382.

SEQ ID NO: 5447 represents the cDNA sequence for clone AA464384.

SEQ ID NO: 5448 represents the cDNA sequence for clone AA464389.

SEQ ID NO: 5449 represents the cDNA sequence for clone AA464391.

SEQ ID NO: 5450 represents the cDNA sequence for clone AA464395.

SEQ ID NO: 5451 represents the cDNA sequence for clone AA464396.

SEQ ID NO: 5452 represents the cDNA sequence for clone AA464397.

SEQ ID NO: 5453 represents the cDNA sequence for clone AA464408.

SEQ ID NO: 5454 represents the cDNA sequence for clone AA464411.

SEQ ID NO: 5455 represents the cDNA sequence for clone AA464426.

SEQ ID NO: 5456 represents the cDNA sequence for clone AA464427.

SEQ ID NO: 5457 represents the cDNA sequence for clone AA464429.

SEQ ID NO: 5458 represents the cDNA sequence for clone AA464440.

SEQ ID NO: 5459 represents the cDNA sequence for clone AA464454.

SEQ ID NO: 5460 represents the cDNA sequence for clone AA464465.

SEQ ID NO: 5461 represents the cDNA sequence for clone AA464468.

SEQ ID NO: 5462 represents the cDNA sequence for clone AA464469.

SEQ ID NO: 5463 represents the cDNA sequence for clone AA464472.

SEQ ID NO: 5464 represents the cDNA sequence for clone AA464488.

SEQ ID NO: 5465 represents the cDNA sequence for clone AA464491.

SEQ ID NO: 5466 represents the cDNA sequence for clone AA464493.

SEQ ID NO: 5467 represents the cDNA sequence for clone AA464510.

SEQ ID NO: 5468 represents the cDNA sequence for clone AA464521.

SEQ ID NO: 5469 represents the cDNA sequence for clone AA464522.

SEQ ID NO: 5470 represents the cDNA sequence for clone AA464525.

SEQ ID NO: 5471 represents the cDNA sequence for clone AA464526.

SEQ ID NO: 5472 represents the cDNA sequence for clone AA464535.

SEQ ID NO: 5473 represents the cDNA sequence for clone AA464538.

SEQ ID NO: 5474 represents the cDNA sequence for clone AA464553.

SEQ ID NO: 5475 represents the cDNA sequence for clone AA464555.

SEQ ID NO: 5476 represents the cDNA sequence for clone AA464558.

SEQ ID NO: 5477 represents the cDNA sequence for clone AA464573.

SEQ ID NO: 5478 represents the cDNA sequence for clone AA464579.

SEQ ID NO: 5479 represents the cDNA sequence for clone AA464633.

SEQ ID NO: 5480 represents the cDNA sequence for clone AA464634.

SEQ ID NO: 5481 represents the cDNA sequence for clone AA464640.

SEQ ID NO: 5482 represents the cDNA sequence for clone AA464641.

SEQ ID NO: 5483 represents the cDNA sequence for clone AA464645.

SEQ ID NO: 5484 represents the cDNA sequence for clone AA464655.

SEQ ID NO: 5485 represents the cDNA sequence for clone AA464658.

SEQ ID NO: 5486 represents the cDNA sequence for clone AA464677.

SEQ ID NO: 5487 represents the cDNA sequence for clone AA464689.

SEQ ID NO: 5488 represents the cDNA sequence for clone AA464691.

SEQ ID NO: 5489 represents the cDNA sequence for clone AA464695.

SEQ ID NO: 5490 represents the cDNA sequence for clone AA464696.

SEQ ID NO: 5491 represents the cDNA sequence for clone AA464698.

SEQ ID NO: 5492 represents the cDNA sequence for clone AA464699.

SEQ ID NO: 5493 represents the cDNA sequence for clone AA464705.

SEQ ID NO: 5494 represents the cDNA sequence for clone AA464707.

SEQ ID NO: 5495 represents the cDNA sequence for clone AA464709.

SEQ ID NO: 5496 represents the cDNA sequence for clone AA464728.

SEQ ID NO: 5497 represents the cDNA sequence for clone AA464733.

SEQ ID NO: 5498 represents the cDNA sequence for clone AA464735.

SEQ ID NO: 5499 represents the cDNA sequence for clone AA464740.

SEQ ID NO: 5500 represents the cDNA sequence for clone AA464742.

SEQ ID NO: 5501 represents the cDNA sequence for clone AA464747.

SEQ ID NO: 5502 represents the cDNA sequence for clone AA464757.

SEQ ID NO: 5503 represents the cDNA sequence for clone AA464758.

SEQ ID NO: 5504 represents the cDNA sequence for clone AA464761.

SEQ ID NO: 5505 represents the cDNA sequence for clone AA464765.

SEQ ID NO: 5506 represents the cDNA sequence for clone AA464766.

SEQ ID NO: 5507 represents the cDNA sequence for clone AA464769.

SEQ ID NO: 5508 represents the cDNA sequence for clone AA464772.

SEQ ID NO: 5509 represents the cDNA sequence for clone AA464774.

SEQ ID NO: 5510 represents the cDNA sequence for clone AA464780.

SEQ ID NO: 5511 represents the cDNA sequence for clone AA464786.

SEQ ID NO: 5512 represents the cDNA sequence for clone AA464791.

SEQ ID NO: 5513 represents the cDNA sequence for clone AA464792.

SEQ ID NO: 5514 represents the cDNA sequence for clone AA464795.

SEQ ID NO: 5515 represents the cDNA sequence for clone AA464952.

SEQ ID NO: 5516 represents the cDNA sequence for clone AA464956.

SEQ ID NO: 5517 represents the cDNA sequence for clone AA464963.

SEQ ID NO: 5518 represents the cDNA sequence for clone AA464964.

SEQ ID NO: 5519 represents the cDNA sequence for clone AA464972.

SEQ ID NO: 5520 represents the cDNA sequence for clone AA464975.

SEQ ID NO: 5521 represents the cDNA sequence for clone AA464976.

SEQ ID NO: 5522 represents the cDNA sequence for clone AA464986.

SEQ ID NO: 5523 represents the cDNA sequence for clone AA464987.

SEQ ID NO: 5524 represents the cDNA sequence for clone AA464988.

SEQ ID NO: 5525 represents the cDNA sequence for clone AA464991.

SEQ ID NO: 5526 represents the cDNA sequence for clone AA464999.

SEQ ID NO: 5527 represents the cDNA sequence for clone AA465002.

SEQ ID NO: 5528 represents the cDNA sequence for clone AA465003.

SEQ ID NO: 5529 represents the cDNA sequence for clone AA465007.

SEQ ID NO: 5530 represents the cDNA sequence for clone AA465011.

SEQ ID NO: 5531 represents the cDNA sequence for clone AA465012.

SEQ ID NO: 5532 represents the cDNA sequence for clone AA465013.

SEQ ID NO: 5533 represents the cDNA sequence for clone AA465014.

SEQ ID NO: 5534 represents the cDNA sequence for clone AA465016.

SEQ ID NO: 5535 represents the cDNA sequence for clone AA465020.

SEQ ID NO: 5536 represents the cDNA sequence for clone AA465022.

SEQ ID NO: 5537 represents the cDNA sequence for clone AA465025.

SEQ ID NO: 5538 represents the cDNA sequence for clone AA465032.

SEQ ID NO: 5539 represents the cDNA sequence for clone AA465033.

SEQ ID NO: 5540 represents the cDNA sequence for clone AA465037.

SEQ ID NO: 5541 represents the cDNA sequence for clone AA465043.

SEQ ID NO: 5542 represents the cDNA sequence for clone AA465044.

SEQ ID NO: 5543 represents the cDNA sequence for clone AA465048.

SEQ ID NO: 5544 represents the cDNA sequence for clone AA465050.

SEQ ID NO: 5545 represents the cDNA sequence for clone AA465052.

SEQ ID NO: 5546 represents the cDNA sequence for clone AA479877.

SEQ ID NO: 5547 represents the cDNA sequence for clone AA476209.

SEQ ID NO: 5548 represents the cDNA sequence for clone AA476210.

SEQ ID NO: 5549 represents the cDNA sequence for clone AA476211.

SEQ ID NO: 5550 represents the cDNA sequence for clone AA476212.

SEQ ID NO: 5551 represents the cDNA sequence for clone AA476213.

SEQ ID NO: 5552 represents the cDNA sequence for clone AA476214.

SEQ ID NO: 5553 represents the cDNA sequence for clone AA476215.

SEQ ID NO: 5554 represents the cDNA sequence for clone AA476220.

SEQ ID NO: 5555 represents the cDNA sequence for clone AA476222.

SEQ ID NO: 5556 represents the cDNA sequence for clone AA476223.

SEQ ID NO: 5557 represents the cDNA sequence for clone AA476227.

SEQ ID NO: 5558 represents the cDNA sequence for clone AA476228.

SEQ ID NO: 5559 represents the cDNA sequence for clone AA476244.

SEQ ID NO: 5560 represents the cDNA sequence for clone AA476276.

SEQ ID NO: 5561 represents the cDNA sequence for clone AA476279.

SEQ ID NO: 5562 represents the cDNA sequence for clone AA476288.

SEQ ID NO: 5563 represents the cDNA sequence for clone AA476297.

SEQ ID NO: 5564 represents the cDNA sequence for clone AA476300.

SEQ ID NO: 5565 represents the cDNA sequence for clone AA476305.

SEQ ID NO: 5566 represents the cDNA sequence for clone AA476848.

SEQ ID NO: 5567 represents the cDNA sequence for clone AA476850.

SEQ ID NO: 5568 represents the cDNA sequence for clone AA476851.

SEQ ID NO: 5569 represents the cDNA sequence for clone AA476862.

SEQ ID NO: 5570 represents the cDNA sequence for clone AA476867.

SEQ ID NO: 5571 represents the cDNA sequence for clone AA476868.

SEQ ID NO: 5572 represents the cDNA sequence for clone AA476869.

SEQ ID NO: 5573 represents the cDNA sequence for clone AA476870.

SEQ ID NO: 5574 represents the cDNA sequence for clone AA476876.

SEQ ID NO: 5575 represents the cDNA sequence for clone AA476881.

SEQ ID NO: 5576 represents the cDNA sequence for clone AA476884.

SEQ ID NO: 5577 represents the cDNA sequence for clone AA476885.

SEQ ID NO: 5578 represents the cDNA sequence for clone AA476891.

SEQ ID NO: 5579 represents the cDNA sequence for clone AA476904.

SEQ ID NO: 5580 represents the cDNA sequence for clone AA476906.

SEQ ID NO: 5581 represents the cDNA sequence for clone AA476907.

SEQ ID NO. 5582 represents the cDNA sequence for clone AA476908.

SEQ ID NO: 5583 represents the cDNA sequence for clone AA476913.

SEQ ID NO: 5584 represents the cDNA sequence for clone AA476915.

SEQ ID NO: 5585 represents the cDNA sequence for clone AA476916.

SEQ ID NO: 5586 represents the cDNA sequence for clone AA476917.

SEQ ID NO: 5587 represents the cDNA sequence for clone AA476918.

SEQ ID NO: 5588 represents the cDNA sequence for clone AA476936.

SEQ ID NO: 5589 represents the cDNA sequence for clone AA476937.

SEQ ID NO: 5590 represents the cDNA sequence for clone AA476943.

SEQ ID NO: 5591 represents the cDNA sequence for clone AA476950.

SEQ ID NO: 5592 represents the cDNA sequence for clone AA476957.

SEQ ID NO: 5593 represents the cDNA sequence for clone AA476963.

SEQ ID NO: 5594 represents the cDNA sequence for clone AA476966.

SEQ ID NO: 5595 represents the cDNA sequence for clone AA476972.

SEQ ID NO: 5596 represents the cDNA sequence for clone AA476973.

SEQ ID NO: 5597 represents the cDNA sequence for clone AA476974.

SEQ ID NO: 5598 represents the cDNA sequence for clone AA476976.

SEQ ID NO: 5599 represents the cDNA sequence for clone AA476977.

SEQ ID NO: 5600 represents the cDNA sequence for clone AA476987.

SEQ ID NO: 5601 represents the cDNA sequence for clone AA476989.

SEQ ID NO: 5602 represents the cDNA sequence for clone AA476993.

SEQ ID NO: 5603 represents the cDNA sequence for clone AA476997.

SEQ ID NO: 5604 represents the cDNA sequence for clone AA477006.

SEQ ID NO: 5605 represents the cDNA sequence for clone AA477008.

SEQ ID NO: 5606 represents the cDNA sequence for clone AA477016.

SEQ ID NO: 5607 represents the cDNA sequence for clone AA477018.

SEQ ID NO: 5608 represents the cDNA sequence for clone AA477025.

SEQ ID NO: 5609 represents the cDNA sequence for clone AA477026.

SEQ ID NO: 5610 represents the cDNA sequence for clone AA477027.

SEQ ID NO: 5611 represents the cDNA sequence for clone AA477031.

SEQ ID NO: 5612 represents the cDNA sequence for clone AA477033.

SEQ ID NO: 5613 represents the cDNA sequence for clone AA477035.

SEQ ID NO: 5614 represents the cDNA sequence for clone AA477041.

SEQ ID NO: 5615 represents the cDNA sequence for clone AA477042.

SEQ ID NO: 5616 represents the cDNA sequence for clone AA477048.

SEQ ID NO: 5617 represents the cDNA sequence for clone AA477049.

SEQ ID NO: 5618 represents the cDNA sequence for clone AA477053.

SEQ ID NO: 5619 represents the cDNA sequence for clone AA477054.

SEQ ID NO: 5620 represents the cDNA sequence for clone AA477059.

SEQ ID NO: 5621 represents the cDNA sequence for clone AA477061.

SEQ ID NO: 5622 represents the cDNA sequence for clone AA477067.

SEQ ID NO: 5623 represents the cDNA sequence for clone AA477075.

SEQ ID NO: 5624 represents the cDNA sequence for clone AA477214.

SEQ ID NO: 5625 represents the cDNA sequence for clone AA477218.

SEQ ID NO: 5626 represents the cDNA sequence for clone AA477224.

SEQ ID NO: 5627 represents the cDNA sequence for clone AA477225.

SEQ ID NO: 5628 represents the cDNA sequence for clone AA477226.

SEQ ID NO: 5629 represents the cDNA sequence for clone AA477231.

SEQ ID NO: 5630 represents the cDNA sequence for clone AA477233.

SEQ ID NO: 5631 represents the cDNA sequence for clone AA477245.

SEQ ID NO: 5632 represents the cDNA sequence for clone AA477249.

SEQ ID NO: 5633 represents the cDNA sequence for clone AA477250.

SEQ ID NO: 5634 represents the cDNA sequence for clone AA477253.

SEQ ID NO: 5635 represents the cDNA sequence for clone AA477256.

SEQ ID NO: 5636 represents the cDNA sequence for clone AA477260.

SEQ ID NO: 5637 represents the cDNA sequence for clone AA477262.

SEQ ID NO: 5638 represents the cDNA sequence for clone AA478977.

SEQ ID NO: 5639 represents the cDNA sequence for clone AA478980.

SEQ ID NO: 5640 represents the cDNA sequence for clone AA478981.

SEQ ID NO: 5641 represents the cDNA sequence for clone AA478984.

SEQ ID NO: 5642 represents the cDNA sequence for clone AA478986.

SEQ ID NO: 5643 represents the cDNA sequence for clone AA478996.

SEQ ID NO: 5644 represents the cDNA sequence for clone AA479005.

SEQ ID NO: 5645 represents the cDNA sequence for clone AA479014.

SEQ ID NO: 5646 represents the cDNA sequence for clone AA479016.

SEQ ID NO: 5647 represents the cDNA sequence for clone AA479017.

SEQ ID NO: 5648 represents the cDNA sequence for clone AA479018.

SEQ ID NO: 5649 represents the cDNA sequence for clone AA479038.

SEQ ID NO: 5650 represents the cDNA sequence for clone AA479048.

SEQ ID NO: 5651 represents the cDNA sequence for clone AA479051.

SEQ ID NO: 5652 represents the cDNA sequence for clone AA479544.

SEQ ID NO: 5653 represents the cDNA sequence for clone AA479551.

SEQ ID NO: 5654 represents the cDNA sequence for clone AA479553.

SEQ ID NO: 5655 represents the cDNA sequence for clone AA479556.

SEQ ID NO: 5656 represents the cDNA sequence for clone AA479568.

SEQ ID NO: 5657 represents the cDNA sequence for clone AA479570.

SEQ ID NO: 5658 represents the cDNA sequence for clone AA479571.

SEQ ID NO: 5659 represents the cDNA sequence for clone AA479572.

SEQ ID NO: 5660 represents the cDNA sequence for clone AA479578.

SEQ ID NO: 5661 represents the cDNA sequence for clone AA479586.

SEQ ID NO: 5662 represents the cDNA sequence for clone AA479591.

SEQ ID NO: 5663 represents the cDNA sequence for clone AA479593.

SEQ ID NO: 5664 represents the cDNA sequence for clone AA479597.

SEQ ID NO: 5665 represents the cDNA sequence for clone AA479606.

SEQ ID NO: 5666 represents the cDNA sequence for clone AA479617.

SEQ ID NO: 5667 represents the cDNA sequence for clone AA479622.

SEQ ID NO: 5668 represents the cDNA sequence for clone AA479623.

SEQ ID NO: 5669 represents the cDNA sequence for clone AA479629.

SEQ ID NO: 5670 represents the cDNA sequence for clone AA479636.

SEQ ID NO: 5671 represents the cDNA sequence for clone AA479637.

SEQ ID NO: 5672 represents the cDNA sequence for clone AA479642.

SEQ ID NO: 5673 represents the cDNA sequence for clone AA479648.

SEQ ID NO: 5674 represents the cDNA sequence for clone AA479653.

SEQ ID NO: 5675 represents the cDNA sequence for clone AA479655.

SEQ ID NO: 5676 represents the cDNA sequence for clone AA479656.

SEQ ID NO: 5677 represents the cDNA sequence for clone AA479665.

SEQ ID NO: 5678 represents the cDNA sequence for clone AA479666.

SEQ ID NO: 5679 represents the cDNA sequence for clone AA479667.

SEQ ID NO: 5680 represents the cDNA sequence for clone AA479671.

SEQ ID NO: 5681 represents the cDNA sequence for clone AA479673.

SEQ ID NO: 5682 represents the cDNA sequence for clone AA479676.

SEQ ID NO: 5683 represents the cDNA sequence for clone AA479678.

SEQ ID NO: 5684 represents the cDNA sequence for clone AA479679.

SEQ ID NO: 5685 represents the cDNA sequence for clone AA479700.

SEQ ID NO: 5686 represents the cDNA sequence for clone AA479704.

SEQ ID NO: 5687 represents the cDNA sequence for clone AA479710.

SEQ ID NO: 5688 represents the cDNA sequence for clone AA479726.

SEQ ID NO: 5689 represents the cDNA sequence for clone AA479729.

SEQ ID NO: 5690 represents the cDNA sequence for clone AA479731.

SEQ ID NO: 5691 represents the cDNA sequence for clone AA479735.

SEQ ID NO: 5692 represents the cDNA sequence for clone AA479744.

SEQ ID NO: 5693 represents the cDNA sequence for clone AA479754.

SEQ ID NO: 5694 represents the cDNA sequence for clone AA479755.

SEQ ID NO: 5695 represents the cDNA sequence for clone AA479774.

SEQ ID NO: 5696 represents the cDNA sequence for clone AA479775.

SEQ ID NO: 5697 represents the cDNA sequence for clone AA479776.

SEQ ID NO: 5698 represents the cDNA sequence for clone AA479787.

SEQ ID NO: 5699 represents the cDNA sequence for clone AA479800.

SEQ ID NO: 5700 represents the cDNA sequence for clone AA479801.

SEQ ID NO: 5701 represents the cDNA sequence for clone AA479802.

SEQ ID NO: 5702 represents the cDNA sequence for clone AA479813.

SEQ ID NO: 5703 represents the cDNA sequence for clone AA479814.

SEQ ID NO: 5704 represents the cDNA sequence for clone AA479817.

SEQ ID NO: 5705 represents the cDNA sequence for clone AA479819.

SEQ ID NO: 5706 represents the cDNA sequence for clone AA479821.

SEQ ID NO: 5707 represents the cDNA sequence for clone AA479823.

SEQ ID NO: 5708 represents the cDNA sequence for clone AA479825.

SEQ ID NO: 5709 represents the cDNA sequence for clone AA479832.

SEQ ID NO: 5710 represents the cDNA sequence for clone AA479848.

SEQ ID NO: 5711 represents the cDNA sequence for clone AA479856.

SEQ ID NO: 5712 represents the cDNA sequence for clone AA479860.

SEQ ID NO: 5713 represents the cDNA sequence for clone AA479861.

SEQ ID NO: 5714 represents the cDNA sequence for clone AA479863.

SEQ ID NO: 5715 represents the cDNA sequence for clone AA479865.

SEQ ID NO: 5716 represents the cDNA sequence for clone AA479867.

SEQ ID NO: 5717 represents the cDNA sequence for clone AA479868.

SEQ ID NO: 5718 represents the cDNA sequence for clone AA479873.

SEQ ID NO: 5719 represents the cDNA sequence for clone AA479874.

SEQ ID NO: 5720 represents the cDNA sequence for clone AA477079.

SEQ ID NO: 5721 represents the cDNA sequence for clone AA477085.

SEQ ID NO: 5722 represents the cDNA sequence for clone AA477089.

SEQ ID NO: 5723 represents the cDNA sequence for clone AA477090.

SEQ ID NO: 5724 represents the cDNA sequence for clone AA477092.

SEQ ID NO: 5725 represents the cDNA sequence for clone AA477095.

SEQ ID NO: 5726 represents the cDNA sequence for clone AA477096.

SEQ ID NO: 5727 represents the cDNA sequence for clone AA477097.

SEQ ID NO: 5728 represents the cDNA sequence for clone AA477099.

SEQ ID NO: 5729 represents the cDNA sequence for clone AA477105.

SEQ ID NO: 5730 represents the cDNA sequence for clone AA477108.

SEQ ID NO: 5731 represents the cDNA sequence for clone AA477109.

SEQ ID NO: 5732 represents the cDNA sequence for clone AA477110.

SEQ ID NO: 5733 represents the cDNA sequence for clone AA477112.

SEQ ID NO: 5734 represents the cDNA sequence for clone AA477115.

SEQ ID NO: 5735 represents the cDNA sequence for clone AA477126.

SEQ ID NO: 5736 represents the cDNA sequence for clone AA477127.

SEQ ID NO: 5737 represents the cDNA sequence for clone AA477131.

SEQ ID NO: 5738 represents the cDNA sequence for clone AA477136.

SEQ ID NO: 5739 represents the cDNA sequence for clone AA477146.

SEQ ID NO: 5740 represents the cDNA sequence for clone AA477148.

SEQ ID NO: 5741 represents the cDNA sequence for clone AA477155.

SEQ ID NO: 5742 represents the cDNA sequence for clone AA477158.

SEQ ID NO: 5743 represents the cDNA sequence for clone AA477159.

SEQ ID NO: 5744 represents the cDNA sequence for clone AA477164.

SEQ ID NO: 5745 represents the cDNA sequence for clone AA477166.

SEQ ID NO: 5746 represents the cDNA sequence for clone AA477169.

SEQ ID NO: 5747 represents the cDNA sequence for clone AA477170.

SEQ ID NO: 5748 represents the cDNA sequence for clone AA477171.

SEQ ID NO: 5749 represents the cDNA sequence for clone AA477176.

SEQ ID NO: 5750 represents the cDNA sequence for clone AA477178.

SEQ ID NO: 5751 represents the cDNA sequence for clone AA477180.

SEQ ID NO: 5752 represents the cDNA sequence for clone AA477181.

SEQ ID NO: 5753 represents the cDNA sequence for clone AA477184.

SEQ ID NO: 5754 represents the cDNA sequence for clone AA477185.

SEQ ID NO: 5755 represents the cDNA sequence for clone AA477191.

SEQ ID NO: 5756 represents the cDNA sequence for clone AA477193.

SEQ ID NO: 5757 represents the cDNA sequence for clone AA477197.

SEQ ID NO: 5758 represents the cDNA sequence for clone AA477200.

SEQ ID NO: 5759 represents the cDNA sequence for clone AA477204.

SEQ ID NO: 5760 represents the cDNA sequence for clone AA477274.

SEQ ID NO: 5761 represents the cDNA sequence for clone AA477275.

SEQ ID NO: 5762 represents the cDNA sequence for clone AA477282.

SEQ ID NO: 5763 represents the cDNA sequence for clone AA477283.

SEQ ID NO: 5764 represents the cDNA sequence for clone AA477284.

SEQ ID NO: 5765 represents the cDNA sequence for clone AA477294.

SEQ ID NO: 5766 represents the cDNA sequence for clone AA477314.

SEQ ID NO: 5767 represents the cDNA sequence for clone AA477330.

SEQ ID NO: 5768 represents the cDNA sequence for clone AA477338.

SEQ ID NO: 5769 represents the cDNA sequence for clone AA477341.

SEQ ID NO: 5770 represents the cDNA sequence for clone AA477349.

SEQ ID NO: 5771 represents the cDNA sequence for clone AA477351.

SEQ ID NO: 5772 represents the cDNA sequence for clone AA477353.

SEQ ID NO: 5773 represents the cDNA sequence for clone AA477354.

SEQ ID NO: 5774 represents the cDNA sequence for clone AA477355.

SEQ ID NO: 5775 represents the cDNA sequence for clone AA477358.

SEQ ID NO: 5776 represents the cDNA sequence for clone AA477361.

SEQ ID NO: 5777 represents the cDNA sequence for clone AA477363.

SEQ ID NO: 5778 represents the cDNA sequence for clone AA477365.

SEQ ID NO: 5779 represents the cDNA sequence for clone AA477369.

SEQ ID NO: 5780 represents the cDNA sequence for clone AA477372.

SEQ ID NO: 5781 represents the cDNA sequence for clone AA477373.

SEQ ID NO: 5782 represents the cDNA sequence for clone AA477385.

SEQ ID NO: 5783 represents the cDNA sequence for clone AA477386.

SEQ ID NO: 5784 represents the cDNA sequence for clone AA477396.

SEQ ID NO: 5785 represents the cDNA sequence for clone AA477405.

SEQ ID NO: 5786 represents the cDNA sequence for clone AA477409.

SEQ ID NO: 5787 represents the cDNA sequence for clone AA477415.

SEQ ID NO: 5788 represents the cDNA sequence for clone AA477425.

SEQ ID NO: 5789 represents the cDNA sequence for clone AA477431.

SEQ ID NO: 5790 represents the cDNA sequence for clone AA477435.

SEQ ID NO: 5791 represents the cDNA sequence for clone AA477460.

SEQ ID NO: 5792 represents the cDNA sequence for clone AA477461.

SEQ ID NO: 5793 represents the cDNA sequence for clone AA477462.

SEQ ID NO: 5794 represents the cDNA sequence for clone AA477470.

SEQ ID NO: 5795 represents the cDNA sequence for clone AA477484.

SEQ ID NO: 5796 represents the cDNA sequence for clone AA477496.

SEQ ID NO: 5797 represents the cDNA sequence for clone AA477501.

SEQ ID NO: 5798 represents the cDNA sequence for clone AA477508.

SEQ ID NO: 5799 represents the cDNA sequence for clone AA477511.

SEQ ID NO: 5800 represents the cDNA sequence for clone AA477514.

SEQ ID NO: 5801 represents the cDNA sequence for clone AA477516.

SEQ ID NO: 5802 represents the cDNA sequence for clone AA477520.

SEQ ID NO: 5803 represents the cDNA sequence for clone AA477521.

SEQ ID NO: 5804 represents the cDNA sequence for clone AA477525.

SEQ ID NO: 5805 represents the cDNA sequence for clone AA477529.

SEQ ID NO: 5806 represents the cDNA sequence for clone AA477531.

SEQ ID NO: 5807 represents the cDNA sequence for clone AA477534.

SEQ ID NO: 5808 represents the cDNA sequence for clone AA477537.

SEQ ID NO: 5809 represents the cDNA sequence for clone AA477547.

SEQ ID NO: 5810 represents the cDNA sequence for clone AA477549.

SEQ ID NO: 5811 represents the cDNA sequence for clone AA477550.

SEQ ID NO: 5812 represents the cDNA sequence for clone AA477564.

SEQ ID NO: 5813 represents the cDNA sequence for clone AA477566.

SEQ ID NO: 5814 represents the cDNA sequence for clone AA477568.

SEQ ID NO: 5815 represents the cDNA sequence for clone AA477587.

SEQ ID NO: 5816 represents the cDNA sequence for clone AA477588.

SEQ ID NO: 5817 represents the cDNA sequence for clone AA477589.

SEQ ID NO: 5818 represents the cDNA sequence for clone AA477591.

SEQ ID NO: 5819 represents the cDNA sequence for clone AA477596.

SEQ ID NO: 5820 represents the cDNA sequence for clone AA477598.

SEQ ID NO: 5821 represents the cDNA sequence for clone AA477601.

SEQ ID NO: 5822 represents the cDNA sequence for clone AA477604.

SEQ ID NO: 5823 represents the cDNA sequence for clone AA477609.

SEQ ID NO: 5824 represents the cDNA sequence for clone AA477616.

SEQ ID NO: 5825 represents the cDNA sequence for clone AA477624.

SEQ ID NO: 5826 represents the cDNA sequence for clone AA477627.

SEQ ID NO: 5827 represents the cDNA sequence for clone AA477631.

SEQ ID NO: 5828 represents the cDNA sequence for clone AA477633.

SEQ ID NO: 5829 represents the cDNA sequence for clone AA477640.

SEQ ID NO: 5830 represents the cDNA sequence for clone AA477661.

SEQ ID NO: 5831 represents the cDNA sequence for clone AA477664.

SEQ ID NO: 5832 represents the cDNA sequence for clone AA477666.

SEQ ID NO: 5833 represents the cDNA sequence for clone AA477667.

SEQ ID NO: 5834 represents the cDNA sequence for clone AA477679.

SEQ ID NO: 5835 represents the cDNA sequence for clone AA477687.

SEQ ID NO: 5836 represents the cDNA sequence for clone AA477688.

SEQ ID NO: 5837 represents the cDNA sequence for clone AA477703.

SEQ ID NO: 5838 represents the cDNA sequence for clone AA477704.

SEQ ID NO: 5839 represents the cDNA sequence for clone AA477705.

SEQ ID NO: 5840 represents the cDNA sequence for clone AA477707.

SEQ ID NO: 5841 represents the cDNA sequence for clone AA477708.

SEQ ID NO: 5842 represents the cDNA sequence for clone AA477715.

SEQ ID NO: 5843 represents the cDNA sequence for clone AA477716.

SEQ ID NO: 5844 represents the cDNA sequence for clone AA477726.

SEQ ID NO: 5845 represents the cDNA sequence for clone AA477727.

SEQ ID NO: 5846 represents the cDNA sequence for clone AA477736.

SEQ ID NO: 5847 represents the cDNA sequence for clone AA477738.

SEQ ID NO: 5848 represents the cDNA sequence for clone AA477749.

SEQ ID NO: 5849 represents the cDNA sequence for clone AA477755.

SEQ ID NO: 5850 represents the cDNA sequence for clone AA477757.

SEQ ID NO: 5851 represents the cDNA sequence for clone AA477760.

SEQ ID NO: 5852 represents the cDNA sequence for clone AA477761.

SEQ ID NO: 5853 represents the cDNA sequence for clone AA477762.

SEQ ID NO: 5854 represents the cDNA sequence for clone AA477776.

SEQ ID NO: 5855 represents the cDNA sequence for clone AA477784.

SEQ ID NO: 5856 represents the cDNA sequence for clone AA477801.

SEQ ID NO: 5857 represents the cDNA sequence for clone AA477815.

SEQ ID NO: 5858 represents the cDNA sequence for clone AA477827.

SEQ ID NO: 5859 represents the cDNA sequence for clone AA477828.

SEQ ID NO: 5860 represents the cDNA sequence for clone AA477841.

SEQ ID NO: 5861 represents the cDNA sequence for clone AA477843.

SEQ ID NO: 5862 represents the cDNA sequence for clone AA477855.

SEQ ID NO: 5863 represents the cDNA sequence for clone AA477859.

SEQ ID NO: 5864 represents the cDNA sequence for clone AA477862.

SEQ ID NO: 5865 represents the cDNA sequence for clone AA477869.

SEQ ID NO: 5866 represents the cDNA sequence for clone AA477870.

SEQ ID NO: 5867 represents the cDNA sequence for clone AA477871.

SEQ ID NO: 5868 represents the cDNA sequence for clone AA477886.

SEQ ID NO: 5869 represents the cDNA sequence for clone AA477887.

SEQ ID NO: 5870 represents the cDNA sequence for clone AA477893.

SEQ ID NO: 5871 represents the cDNA sequence for clone AA477894.

SEQ ID NO: 5872 represents the cDNA sequence for clone AA477904.

SEQ ID NO: 5873 represents the cDNA sequence for clone AA477908.

SEQ ID NO: 5874 represents the cDNA sequence for clone AA477911.

SEQ ID NO: 5875 represents the cDNA sequence for clone AA477914.

SEQ ID NO: 5876 represents the cDNA sequence for clone AA477915.

SEQ ID NO: 5877 represents the cDNA sequence for clone AA477917.

SEQ ID NO: 5878 represents the cDNA sequence for clone AA477918.

SEQ ID NO: 5879 represents the cDNA sequence for clone AA477919.

SEQ ID NO: 5880 represents the cDNA sequence for clone AA477923.

SEQ ID NO: 5881 represents the cDNA sequence for clone AA477928.

SEQ ID NO: 5882 represents the cDNA sequence for clone AA477932.

SEQ ID NO: 5883 represents the cDNA sequence for clone AA477941.

SEQ ID NO: 5884 represents the cDNA sequence for clone AA477954.

SEQ ID NO: 5885 represents the cDNA sequence for clone AA477956.

SEQ ID NO: 5886 represents the cDNA sequence for clone AA477961.

SEQ ID NO: 5887 represents the cDNA sequence for clone AA477962.

SEQ ID NO: 5888 represents the cDNA sequence for clone AA477968.

SEQ ID NO: 5889 represents the cDNA sequence for clone AA477970.

SEQ ID NO: 5890 represents the cDNA sequence for clone AA477971.

SEQ ID NO: 5891 represents the cDNA sequence for clone AA477972.

SEQ ID NO: 5892 represents the cDNA sequence for clone AA477988.

SEQ ID NO: 5893 represents the cDNA sequence for clone AA477989.

SEQ ID NO: 5894 represents the cDNA sequence for clone AA478000.

SEQ ID NO: 5895 represents the cDNA sequence for clone AA478015.

SEQ ID NO: 5896 represents the cDNA sequence for clone AA478023.

SEQ ID NO: 5897 represents the cDNA sequence for clone AA478032.

SEQ ID NO: 5898 represents the cDNA sequence for clone AA478040.

SEQ ID NO: 5899 represents the cDNA sequence for clone AA478049.

SEQ ID NO: 5900 represents the cDNA sequence for clone AA478054.

SEQ ID NO: 5901 represents the cDNA sequence for clone AA478055.

SEQ ID NO: 5902 represents the cDNA sequence for clone AA478071.

SEQ ID NO: 5903 represents the cDNA sequence for clone AA478072.

SEQ ID NO: 5904 represents the cDNA sequence for clone AA478076.

SEQ ID NO: 5905 represents the cDNA sequence for clone AA478079.

SEQ ID NO: 5906 represents the cDNA sequence for clone AA478080.

SEQ ID NO: 5907 represents the cDNA sequence for clone AA478083.

SEQ ID NO: 5908 represents the cDNA sequence for clone AA478085.

SEQ ID NO: 5909 represents the cDNA sequence for clone AA478089.

SEQ ID NO: 5910 represents the cDNA sequence for clone AA478091.

SEQ ID NO: 5911 represents the cDNA sequence for clone AA478122.

SEQ ID NO: 5912 represents the cDNA sequence for clone AA478123.

SEQ ID NO: 5913 represents the cDNA sequence for clone AA478125.

SEQ ID NO: 5914 represents the cDNA sequence for clone AA478133.

SEQ ID NO: 5915 represents the cDNA sequence for clone AA478134.

SEQ ID NO: 5916 represents the cDNA sequence for clone AA478155.

SEQ ID NO: 5917 represents the cDNA sequence for clone AA478158.

SEQ ID NO: 5918 represents the cDNA sequence for clone AA478161.

SEQ ID NO: 5919 represents the cDNA sequence for clone AA478162.

SEQ ID NO: 5920 represents the cDNA sequence for clone AA478171.

SEQ ID NO: 5921 represents the cDNA sequence for clone AA478175.

SEQ ID NO: 5922 represents the cDNA sequence for clone AA478178.

SEQ ID NO: 5923 represents the cDNA sequence for clone AA478195.

SEQ ID NO: 5924 represents the cDNA sequence for clone AA478198.

SEQ ID NO: 5925 represents the cDNA sequence for clone AA478203.

SEQ ID NO: 5926 represents the cDNA sequence for clone AA478205.

SEQ ID NO: 5927 represents the cDNA sequence for clone AA478209.

SEQ ID NO: 5928 represents the cDNA sequence for clone AA478210.

SEQ ID NO: 5929 represents the cDNA sequence for clone AA478211.

SEQ ID NO: 5930 represents the cDNA sequence for clone AA478216.

SEQ ID NO: 5931 represents the cDNA sequence for clone AA478222.

SEQ ID NO: 5932 represents the cDNA sequence for clone AA478224.

SEQ ID NO: 5933 represents the cDNA sequence for clone AA478226.

SEQ ID NO: 5934 represents the cDNA sequence for clone AA478228.

SEQ ID NO: 5935 represents the cDNA sequence for clone AA478229.

SEQ ID NO: 5936 represents the cDNA sequence for clone AA478235.

SEQ ID NO: 5937 represents the cDNA sequence for clone AA478246.

SEQ ID NO: 5938 represents the cDNA sequence for clone AA478247.

SEQ ID NO: 5939 represents the cDNA sequence for clone AA478257.

SEQ ID NO: 5940 represents the cDNA sequence for clone AA478259.

SEQ ID NO: 5941 represents the cDNA sequence for clone AA478265.

SEQ ID NO: 5942 represents the cDNA sequence for clone AA478269.

SEQ ID NO: 5943 represents the cDNA sequence for clone AA478274.

SEQ ID NO: 5944 represents the cDNA sequence for clone AA478279.

SEQ ID NO: 5945 represents the cDNA sequence for clone AA478283.

SEQ ID NO: 5946 represents the cDNA sequence for clone AA478284.

SEQ ID NO: 5947 represents the cDNA sequence for clone AA478289.

SEQ ID NO: 5948 represents the cDNA sequence for clone AA478296.

SEQ ID NO: 5949 represents the cDNA sequence for clone AA478305.

SEQ ID NO: 5950 represents the cDNA sequence for clone AA478309.

SEQ ID NO: 5951 represents the cDNA sequence for clone AA478318.

SEQ ID NO: 5952 represents the cDNA sequence for clone AA478321.

SEQ ID NO: 5953 represents the cDNA sequence for clone AA478325.

SEQ ID NO: 5954 represents the cDNA sequence for clone AA478332.

SEQ ID NO: 5955 represents the cDNA sequence for clone AA478333.

SEQ ID NO: 5956 represents the cDNA sequence for clone AA478338.

SEQ ID NO: 5957 represents the cDNA sequence for clone AA478339.

SEQ ID NO: 5958 represents the cDNA sequence for clone AA478342.

SEQ ID NO: 5959 represents the cDNA sequence for clone AA478347.

SEQ ID NO: 5960 represents the cDNA sequence for clone AA478354.

SEQ ID NO: 5961 represents the cDNA sequence for clone AA478364.

SEQ ID NO: 5962 represents the cDNA sequence for clone AA478368.

SEQ ID NO: 5963 represents the cDNA sequence for clone AA478369.

SEQ ID NO: 5964 represents the cDNA sequence for clone AA478373.

SEQ ID NO: 5965 represents the cDNA sequence for clone AA478375.

SEQ ID NO: 5966 represents the cDNA sequence for clone AA478378.

SEQ ID NO: 5967 represents the cDNA sequence for clone AA478379.

SEQ ID NO: 5968 represents the cDNA sequence for clone AA478381.

SEQ ID NO: 5969 represents the cDNA sequence for clone AA478383.

SEQ ID NO: 5970 represents the cDNA sequence for clone AA478388.

SEQ ID NO: 5971 represents the cDNA sequence for clone AA478397.

SEQ ID NO: 5972 represents the cDNA sequence for clone AA478398.

SEQ ID NO: 5973 represents the cDNA sequence for clone AA478401.

SEQ ID NO: 5974 represents the cDNA sequence for clone AA478416.

SEQ ID NO: 5975 represents the cDNA sequence for clone AA478417.

SEQ ID NO: 5976 represents the cDNA sequence for clone AA478418.

SEQ ID NO: 5977 represents the cDNA sequence for clone AA478423.

SEQ ID NO: 5978 represents the cDNA sequence for clone AA478429.

SEQ ID NO: 5979 represents the cDNA sequence for clone AA480054.

SEQ ID NO: 5980 represents the cDNA sequence for clone AA480066.

SEQ ID NO: 5981 represents the cDNA sequence for clone AA480069.

SEQ ID NO: 5982 represents the cDNA sequence for clone AA480071.

SEQ ID NO: 5983 represents the cDNA sequence for clone AA480072.

SEQ ID NO: 5984 represents the cDNA sequence for clone AA480077.

SEQ ID NO: 5985 represents the cDNA sequence for clone AA480083.

SEQ ID NO: 5986 represents the cDNA sequence for clone AA480084.

SEQ ID NO: 5987 represents the cDNA sequence for clone AA480090.

SEQ ID NO: 5988 represents the cDNA sequence for clone AA480094.

SEQ ID NO: 5989 represents the cDNA sequence for clone AA480116.

SEQ ID NO: 5990 represents the cDNA sequence for clone AA480123.

SEQ ID NO: 5991 represents the cDNA sequence for clone AA480125.

SEQ ID NO: 5992 represents the cDNA sequence for clone AA480126.

SEQ ID NO: 5993 represents the cDNA sequence for clone AA480128.

SEQ ID NO: 5994 represents the cDNA sequence for clone AA480129.

SEQ ID NO: 5995 represents the cDNA sequence for clone AA480135.

SEQ ID NO: 5996 represents the cDNA sequence for clone AA480145.

SEQ ID NO: 5997 represents the cDNA sequence for clone AA481818.

SEQ ID NO: 5998 represents the cDNA sequence for clone AA481844.

SEQ ID NO: 5999 represents the cDNA sequence for clone AA481848.

SEQ ID NO: 6000 represents the cDNA sequence for clone AA481850.

SEQ ID NO: 6001 represents the cDNA sequence for clone AA481855.

SEQ ID NO: 6002 represents the cDNA sequence for clone AA481857.

SEQ ID NO: 6003 represents the cDNA sequence for clone AA481859.

SEQ ID NO: 6004 represents the cDNA sequence for clone AA481860.

SEQ ID NO: 6005 represents the cDNA sequence for clone AA481864.

SEQ ID NO: 6006 represents the cDNA sequence for clone AA481870.

SEQ ID NO: 6007 represents the cDNA sequence for clone AA481879.

SEQ ID NO: 6008 represents the cDNA sequence for clone AA481887.

SEQ ID NO: 6009 represents the cDNA sequence for clone AA481908.

SEQ ID NO: 6010 represents the cDNA sequence for clone AA481918.

SEQ ID NO: 6011 represents the cDNA sequence for clone AA481921.

SEQ ID NO: 6012 represents the cDNA sequence for clone AA481925.

SEQ ID NO: 6013 represents the cDNA sequence for clone AA481927.

SEQ ID NO: 6014 represents the cDNA sequence for clone AA481928.

SEQ ID NO: 6015 represents the cDNA sequence for clone AA481929.

SEQ ID NO: 6016 represents the cDNA sequence for clone AA481935.

SEQ ID NO: 6017 represents the cDNA sequence for clone AA481939.

SEQ ID NO: 6018 represents the cDNA sequence for clone AA481947.

SEQ ID NO: 6019 represents the cDNA sequence for clone AA481948.

SEQ ID NO: 6020 represents the cDNA sequence for clone AA481949.

SEQ ID NO: 6021 represents the cDNA sequence for clone AA481952.

SEQ ID NO: 6022 represents the cDNA sequence for clone AA481954.

SEQ ID NO: 6023 represents the cDNA sequence for clone AA481955.

SEQ ID NO: 6024 represents the cDNA sequence for clone AA481956.

SEQ ID NO: 6025 represents the cDNA sequence for clone AA481958.

SEQ ID NO: 6026 represents the cDNA sequence for clone AA481960.

SEQ ID NO: 6027 represents the cDNA sequence for clone AA481962.

SEQ ID NO: 6028 represents the cDNA sequence for clone AA481964.

SEQ ID NO: 6029 represents the cDNA sequence for clone AA481971.

SEQ ID NO: 6030 represents the cDNA sequence for clone AA481972.

SEQ ID NO: 6031 represents the cDNA sequence for clone AA481975.

SEQ ID NO: 6032 represents the cDNA sequence for clone AA481978.

SEQ ID NO: 6033 represents the cDNA sequence for clone AA481981.

SEQ ID NO: 6034 represents the cDNA sequence for clone AA481988.

SEQ ID NO: 6035 represents the cDNA sequence for clone AA481997.

SEQ ID NO: 6036 represents the cDNA sequence for clone AA482040.

SEQ ID NO: 6037 represents the cDNA sequence for clone AA482041.

SEQ ID NO: 6038 represents the cDNA sequence for clone AA482045.

SEQ ID NO: 6039 represents the cDNA sequence for clone AA482046.

SEQ ID NO: 6040 represents the cDNA sequence for clone AA482048.

SEQ ID NO: 6041 represents the cDNA sequence for clone AA482054.

SEQ ID NO: 6042 represents the cDNA sequence for clone AA482061.

SEQ ID NO: 6043 represents the cDNA sequence for clone AA482062.

SEQ ID NO: 6044 represents the cDNA sequence for clone AA482069.

SEQ ID NO: 6045 represents the cDNA sequence for clone AA482072.

SEQ ID NO: 6046 represents the cDNA sequence for clone AA482073.

SEQ ID NO: 6047 represents the cDNA sequence for clone AA482075.

SEQ ID NO: 6048 represents the cDNA sequence for clone AA482077.

SEQ ID NO: 6049 represents the cDNA sequence for clone AA482081.

SEQ ID NO: 6050 represents the cDNA sequence for clone AA482082.

SEQ ID NO: 6051 represents the cDNA sequence for clone AA482086.

SEQ ID NO: 6052 represents the cDNA sequence for clone AA482088.

SEQ ID NO: 6053 represents the cDNA sequence for clone AA482095.

SEQ ID NO: 6054 represents the cDNA sequence for clone AA482109.

SEQ ID NO: 6055 represents the cDNA sequence for clone AA482112.

SEQ ID NO: 6056 represents the cDNA sequence for clone AA482113.

SEQ ID NO: 6057 represents the cDNA sequence for clone AA482398.

SEQ ID NO: 6058 represents the cDNA sequence for clone AA482399.

SEQ ID NO: 6059 represents the cDNA sequence for clone AA482401.

SEQ ID NO: 6060 represents the cDNA sequence for clone AA482403.

SEQ ID NO: 6061 represents the cDNA sequence for clone AA482409.

SEQ ID NO: 6062 represents the cDNA sequence for clone AA482410.

SEQ ID NO: 6063 represents the cDNA sequence for clone AA482411.

SEQ ID NO: 6064 represents the cDNA sequence for clone AA482412.

SEQ ID NO: 6065 represents the cDNA sequence for clone AA482413.

SEQ ID NO: 6066 represents the cDNA sequence for clone AA482424.

SEQ ID NO: 6067 represents the cDNA sequence for clone AA482427.

SEQ ID NO: 6068 represents the cDNA sequence for clone AA482428.

SEQ ID NO: 6069 represents the cDNA sequence for clone AA482431.

SEQ ID NO: 6070 represents the cDNA sequence for clone AA482481.

SEQ ID NO: 6071 represents the cDNA sequence for clone AA482482.

SEQ ID NO: 6072 represents the cDNA sequence for clone AA482487.

SEQ ID NO: 6073 represents the cDNA sequence for clone AA482489.

SEQ ID NO: 6074 represents the cDNA sequence for clone AA482499.

SEQ ID NO: 6075 represents the cDNA sequence for clone AA482503.

SEQ ID NO: 6076 represents the cDNA sequence for clone AA482508.

SEQ ID NO: 6077 represents the cDNA sequence for clone AA482544.

SEQ ID NO: 6078 represents the cDNA sequence for clone AA482545.

SEQ ID NO: 6079 represents the cDNA sequence for clone AA482548.

SEQ ID NO: 6080 represents the cDNA sequence for clone AA482557.

SEQ ID NO: 6081 represents the cDNA sequence for clone AA482600.

SEQ ID NO: 6082 represents the cDNA sequence for clone AA482618.

SEQ ID NO: 6083 represents the cDNA sequence for clone AA482629.

SEQ ID NO: 6084 represents the cDNA sequence for clone AA482631.

SEQ ID NO: 6085 represents the cDNA sequence for clone AA482636.

SEQ ID NO: 6086 represents the cDNA sequence for clone AA482637.

SEQ ID NO: 6087 represents the cDNA sequence for clone AA482638.

SEQ ID NO: 6088 represents the cDNA sequence for clone AA482639.

SEQ ID NO: 6089 represents the cDNA sequence for clone AA482642.

SEQ ID NO: 6090 represents the cDNA sequence for clone AA482647.

SEQ ID NO: 6091 represents the cDNA sequence for clone AA482650.

SEQ ID NO: 6092 represents the cDNA sequence for clone AA482651.

SEQ ID NO: 6093 represents the cDNA sequence for clone AA482653.

SEQ ID NO: 6094 represents the cDNA sequence for clone AA482660.

SEQ ID NO: 6095 represents the cDNA sequence for clone AA482662.

SEQ ID NO: 6096 represents the cDNA sequence for clone AA482666.

SEQ ID NO: 6097 represents the cDNA sequence for clone AA480806.

SEQ ID NO: 6098 represents the cDNA sequence for clone AA480816.

SEQ ID NO: 6099 represents the cDNA sequence for clone AA480818.

SEQ ID NO: 6100 represents the cDNA sequence for clone AA480827.

SEQ ID NO: 6101 represents the cDNA sequence for clone AA480836.

SEQ ID NO: 6102 represents the cDNA sequence for clone AA480847.

SEQ ID NO: 6103 represents the cDNA sequence for clone AA481010.

SEQ ID NO: 6104 represents the cDNA sequence for clone AA481012.

SEQ ID NO: 6105 represents the cDNA sequence for clone AA481017.

SEQ ID NO: 6106 represents the cDNA sequence for clone AA481018.

SEQ ID NO: 6107 represents the cDNA sequence for clone AA481020.

SEQ ID NO: 6108 represents the cDNA sequence for clone AA481021.

SEQ ID NO: 6109 represents the cDNA sequence for clone AA481342.

SEQ ID NO: 6110 represents the cDNA sequence for clone AA481345.

SEQ ID NO: 6111 represents the cDNA sequence for clone AA481348.

SEQ ID NO: 6112 represents the cDNA sequence for clone AA481352.

SEQ ID NO: 6113 represents the cDNA sequence for clone AA481356.

SEQ ID NO: 6114 represents the cDNA sequence for clone AA481357.

SEQ ID NO: 6115 represents the cDNA sequence for clone AA481363.

SEQ ID NO: 6116 represents the cDNA sequence for clone AA481369.

SEQ ID NO: 6117 represents the cDNA sequence for clone AA481375.

SEQ ID NO: 6118 represents the cDNA sequence for clone AA481384.

SEQ ID NO: 6119 represents the cDNA sequence for clone AA481448.

SEQ ID NO: 6120 represents the cDNA sequence for clone AA481451.

SEQ ID NO: 6121 represents the cDNA sequence for clone AA481453.

SEQ ID NO: 6122 represents the cDNA sequence for clone AA481454.

SEQ ID NO: 6123 represents the cDNA sequence for clone AA481455.

SEQ ID NO: 6124 represents the cDNA sequence for clone AA481456.

SEQ ID NO: 6125 represents the cDNA sequence for clone AA481467.

SEQ ID NO: 6126 represents the cDNA sequence for clone AA481476.

SEQ ID NO: 6127 represents the cDNA sequence for clone AA481482.

SEQ ID NO: 6128 represents the cDNA sequence for clone AA481483.

SEQ ID NO: 6129 represents the cDNA sequence for clone AA481687.

SEQ ID NO: 6130 represents the cDNA sequence for clone AA481689.

SEQ ID NO: 6131 represents the cDNA sequence for clone AA481690.

SEQ ID NO: 6132 represents the cDNA sequence for clone AA481691.

SEQ ID NO: 6133 represents the cDNA sequence for clone AA481692.

SEQ ID NO: 6134 represents the cDNA sequence for clone AA481693.

SEQ ID NO: 6135 represents the cDNA sequence for clone AA481703.

SEQ ID NO: 6136 represents the cDNA sequence for clone AA481709.

SEQ ID NO: 6137 represents the cDNA sequence for clone AA481714.

SEQ ID NO: 6138 represents the cDNA sequence for clone AA481717.

SEQ ID NO: 6139 represents the cDNA sequence for clone AA481719.

SEQ ID NO: 6140 represents the cDNA sequence for clone AA481720.

SEQ ID NO: 6141 represents the cDNA sequence for clone AA481721.

SEQ ID NO: 6142 represents the cDNA sequence for clone AA481744.

SEQ ID NO: 6143 represents the cDNA sequence for clone AA481753.

SEQ ID NO: 6144 represents the cDNA sequence for clone AA481754.

SEQ ID NO: 6145 represents the cDNA sequence for clone AA481757.

SEQ ID NO: 6146 represents the cDNA sequence for clone AA485351.

SEQ ID NO: 6147 represents the cDNA sequence for clone AA485360.

SEQ ID NO: 6148 represents the cDNA sequence for clone AA485369.

SEQ ID NO: 6149 represents the cDNA sequence for clone AA485370.

SEQ ID NO: 6150 represents the cDNA sequence for clone AA485378.

SEQ ID NO: 6151 represents the cDNA sequence for clone AA485381.

SEQ ID NO: 6152 represents the cDNA sequence for clone AA485422.

SEQ ID NO: 6153 represents the cDNA sequence for clone AA485425.

SEQ ID NO: 6154 represents the cDNA sequence for clone AA485432.

SEQ ID NO: 6155 represents the cDNA sequence for clone AA485438.

SEQ ID NO: 6156 represents the cDNA sequence for clone AA485451.

SEQ ID NO: 6157 represents the cDNA sequence for clone AA485454.

SEQ ID NO: 6158 represents the cDNA sequence for clone AA485455.

SEQ ID NO: 6159 represents the cDNA sequence for clone AA485504.

SEQ ID NO: 6160 represents the cDNA sequence for clone AA485507.

SEQ ID NO: 6161 represents the cDNA sequence for clone AA485511.

SEQ ID NO: 6162 represents the cDNA sequence for clone AA485515.

SEQ ID NO: 6163 represents the cDNA sequence for clone AA485516.

SEQ ID NO: 6164 represents the cDNA sequence for clone AA485518.

SEQ ID NO: 6165 represents the cDNA sequence for clone AA485520.

SEQ ID NO: 6166 represents the cDNA sequence for clone AA485526.

SEQ ID NO: 6167 represents the cDNA sequence for clone AA485527.

SEQ ID NO: 6168 represents the cDNA sequence for clone AA485529.

SEQ ID NO: 6169 represents the cDNA sequence for clone AA485538.

SEQ ID NO: 6170 represents the cDNA sequence for clone AA485585.

SEQ ID NO: 6171 represents the cDNA sequence for clone AA485586.

SEQ ID NO: 6172 represents the cDNA sequence for clone AA485589.

SEQ ID NO: 6173 represents the cDNA sequence for clone AA485592.

SEQ ID NO: 6174 represents the cDNA sequence for clone AA485596.

SEQ ID NO: 6175 represents the cDNA sequence for clone AA485601.

SEQ ID NO: 6176 represents the cDNA sequence for clone AA485602.

SEQ ID NO: 6177 represents the cDNA sequence for clone AA485604.

SEQ ID NO: 6178 represents the cDNA sequence for clone AA485611.

SEQ ID NO: 6179 represents the cDNA sequence for clone AA485619.

SEQ ID NO: 6180 represents the cDNA sequence for clone AA485622.

SEQ ID NO: 6181 represents the cDNA sequence for clone AA485665.

SEQ ID NO: 6182 represents the cDNA sequence for clone AA485669.

SEQ ID NO: 6183 represents the cDNA sequence for clone AA485676.

SEQ ID NO: 6184 represents the cDNA sequence for clone AA485678.

SEQ ID NO: 6185 represents the cDNA sequence for clone AA485679.

SEQ ID NO: 6186 represents the cDNA sequence for clone AA485683.

SEQ ID NO: 6187 represents the cDNA sequence for clone AA485732.

SEQ ID NO: 6188 represents the cDNA sequence for clone AA485735.

SEQ ID NO: 6189 represents the cDNA sequence for clone AA485749.

SEQ ID NO: 6190 represents the cDNA sequence for clone AA485799.

SEQ ID NO: 6191 represents the cDNA sequence for clone AA485800.

SEQ ID NO: 6192 represents the cDNA sequence for clone AA485803.

SEQ ID NO: 6193 represents the cDNA sequence for clone AA485804.

SEQ ID NO: 6194 represents the cDNA sequence for clone AA485806.

SEQ ID NO: 6195 represents the cDNA sequence for clone AA485810.

SEQ ID NO: 6196 represents the cDNA sequence for clone AA485813.

SEQ ID NO: 6197 represents the cDNA sequence for clone AA485818.

SEQ ID NO: 6198 represents the cDNA sequence for clone AA486451.

SEQ ID NO: 6199 represents the cDNA sequence for clone AA486453.

SEQ ID NO: 6200 represents the cDNA sequence for clone AA486454.

SEQ ID NO: 6201 represents the cDNA sequence for clone AA486456.

SEQ ID NO: 6202 represents the cDNA sequence for clone AA486458.

SEQ ID NO: 6203 represents the cDNA sequence for clone AA486462.

SEQ ID NO: 6204 represents the cDNA sequence for clone AA486469.

SEQ ID NO: 6205 represents the cDNA sequence for clone AA496280.

SEQ ID NO: 6206 represents the cDNA sequence for clone AA496281.

SEQ ID NO: 6207 represents the cDNA sequence for clone AA496282.

SEQ ID NO: 6208 represents the cDNA sequence for clone AA496285.

SEQ ID NO: 6209 represents the cDNA sequence for clone AA496286.

SEQ ID NO: 6210 represents the cDNA sequence for clone AA496289.

SEQ ID NO: 6211 represents the cDNA sequence for clone AA496295.

SEQ ID NO: 6212 represents the cDNA sequence for clone AA496302.

SEQ ID NO: 6213 represents the cDNA sequence for clone AA496304.

SEQ ID NO: 6214 represents the cDNA sequence for clone AA496306.

SEQ ID NO: 6215 represents the cDNA sequence for clone AA496320.

SEQ ID NO: 6216 represents the cDNA sequence for clone AA496323.

SEQ ID NO: 6217 represents the cDNA sequence for clone AA496326.

SEQ ID NO: 6218 represents the cDNA sequence for clone AA496339.

SEQ ID NO: 6219 represents the cDNA sequence for clone AA496340.

SEQ ID NO: 6220 represents the cDNA sequence for clone AA496341.

SEQ ID NO: 6221 represents the cDNA sequence for clone AA496349.

SEQ ID NO: 6222 represents the cDNA sequence for clone AA496362.

SEQ ID NO: 6223 represents the cDNA sequence for clone AA496365.

SEQ ID NO: 6224 represents the cDNA sequence for clone AA496369.

SEQ ID NO: 6225 represents the cDNA sequence for clone AA496374.

SEQ ID NO: 6226 represents the cDNA sequence for clone AA496379.

SEQ ID NO: 6227 represents the cDNA sequence for clone AA496389.

SEQ ID NO: 6228 represents the cDNA sequence for clone AA496390.

SEQ ID NO: 6229 represents the cDNA sequence for clone AA496398.

SEQ ID NO: 6230 represents the cDNA sequence for clone AA496409.

SEQ ID NO: 6231 represents the cDNA sequence for clone AA496413.

SEQ ID NO: 6232 represents the cDNA sequence for clone AA496419.

SEQ ID NO: 6233 represents the cDNA sequence for clone AA496424.

SEQ ID NO: 6234 represents the cDNA sequence for clone AA496432.

SEQ ID NO: 6235 represents the cDNA sequence for clone AA496435.

SEQ ID NO: 6236 represents the cDNA sequence for clone AA496444.

SEQ ID NO: 6237 represents the cDNA sequence for clone AA496455.

SEQ ID NO: 6238 represents the cDNA sequence for clone AA496457.

SEQ ID NO: 6239 represents the cDNA sequence for clone AA496459.

SEQ ID NO: 6240 represents the cDNA sequence for clone AA496461.

SEQ ID NO: 6241 represents the cDNA sequence for clone AA496462.

SEQ ID NO: 6242 represents the cDNA sequence for clone AA496464.

SEQ ID NO: 6243 represents the cDNA sequence for clone AA496479.

SEQ ID NO: 6244 represents the cDNA sequence for clone AA496480.

SEQ ID NO: 6245 represents the cDNA sequence for clone AA496481.

SEQ ID NO: 6246 represents the cDNA sequence for clone AA496483.

SEQ ID NO: 6247 represents the cDNA sequence for clone AA496484.

SEQ ID NO: 6248 represents the cDNA sequence for clone AA496486.

SEQ ID NO: 6249 represents the cDNA sequence for clone AA496491.

SEQ ID NO: 6250 represents the cDNA sequence for clone AA496497.

SEQ ID NO: 6251 represents the cDNA sequence for clone AA496499.

SEQ ID NO: 6252 represents the cDNA sequence for clone AA496500.

SEQ ID NO: 6253 represents the cDNA sequence for clone AA496503.

SEQ ID NO: 6254 represents the cDNA sequence for clone AA496513.

SEQ ID NO: 6255 represents the cDNA sequence for clone AA496529.

SEQ ID NO: 6256 represents the cDNA sequence for clone AA496532.

SEQ ID NO: 6257 represents the cDNA sequence for clone AA496537.

SEQ ID NO: 6258 represents the cDNA sequence for clone AA496539.

SEQ ID NO: 6259 represents the cDNA sequence for clone AA496542.

SEQ ID NO: 6260 represents the cDNA sequence for clone AA496543.

SEQ ID NO: 6261 represents the cDNA sequence for clone AA496551.

SEQ ID NO: 6262 represents the cDNA sequence for clone AA496552.

SEQ ID NO: 6263 represents the cDNA sequence for clone AA496557.

SEQ ID NO: 6264 represents the cDNA sequence for clone AA496577.

SEQ ID NO: 6265 represents the cDNA sequence for clone AA496586.

SEQ ID NO: 6266 represents the cDNA sequence for clone AA496589.

SEQ ID NO: 6267 represents the cDNA sequence for clone AA496597.

SEQ ID NO: 6268 represents the cDNA sequence for clone AA496600.

SEQ ID NO: 6269 represents the cDNA sequence for clone AA496602.

SEQ ID NO: 6270 represents the cDNA sequence for clone AA496607.

SEQ ID NO: 6271 represents the cDNA sequence for clone AA496608.

SEQ ID NO: 6272 represents the cDNA sequence for clone AA496613.

SEQ ID NO: 6273 represents the cDNA sequence for clone AA496614.

SEQ ID NO: 6274 represents the cDNA sequence for clone AA496622.

SEQ ID NO: 6275 represents the cDNA sequence for clone AA496634.

SEQ ID NO: 6276 represents the cDNA sequence for clone AA496635.

SEQ ID NO: 6277 represents the cDNA sequence for clone AA496643.

SEQ ID NO: 6278 represents the cDNA sequence for clone AA496646.

SEQ ID NO: 6279 represents the cDNA sequence for clone AA496651.

SEQ ID NO: 6280 represents the cDNA sequence for clone AA496653.

SEQ ID NO: 6281 represents the cDNA sequence for clone AA496658.

SEQ ID NO: 6282 represents the cDNA sequence for clone AA496668.

SEQ ID NO: 6283 represents the cDNA sequence for clone AA502403.

SEQ ID NO: 6284 represents the cDNA sequence for clone AA502404.

SEQ ID NO: 6285 represents the cDNA sequence for clone AA502410.

SEQ ID NO: 6286 represents the cDNA sequence for clone AA502415.

SEQ ID NO: 6287 represents the cDNA sequence for clone AA502416.

SEQ ID NO: 6288 represents the cDNA sequence for clone AA502419.

SEQ ID NO: 6289 represents the cDNA sequence for clone AA502420.

SEQ ID NO: 6290 represents the cDNA sequence for clone AA502421.

SEQ ID NO: 6291 represents the cDNA sequence for clone AA502422.

SEQ ID NO: 6292 represents the cDNA sequence for clone AA502424.

SEQ ID NO: 6293 represents the cDNA sequence for clone AA502425.

SEQ ID NO: 6294 represents the cDNA sequence for clone AA502506.

SEQ ID NO: 6295 represents the cDNA sequence for clone AA502507.

SEQ ID NO: 6296 represents the cDNA sequence for clone AA502509.

SEQ ID NO: 6297 represents the cDNA sequence for clone AA502512.

SEQ ID NO: 6298 represents the cDNA sequence for clone AA502514.

SEQ ID NO: 6299 represents the cDNA sequence for clone AA502516.

SEQ ID NO: 6300 represents the cDNA sequence for clone AA502518.

SEQ ID NO: 6301 represents the cDNA sequence for clone AA502519.

SEQ ID NO: 6302 represents the cDNA sequence for clone AA502521.

SEQ ID NO: 6303 represents the cDNA sequence for clone AA502522.

SEQ ID NO: 6304 represents the cDNA sequence for clone AA502527.

SEQ ID NO: 6305 represents the cDNA sequence for clone AA502530.

SEQ ID NO: 6306 represents the cDNA sequence for clone AA502531.

SEQ ID NO: 6307 represents the cDNA sequence for clone AA502533.

SEQ ID NO: 6308 represents the cDNA sequence for clone AA502534.

SEQ ID NO: 6309 represents the cDNA sequence for clone AA502536.

SEQ ID NO: 6310 represents the cDNA sequence for clone AA502537.

SEQ ID NO: 6311 represents the cDNA sequence for clone AA502539.

SEQ ID NO: 6312 represents the cDNA sequence for clone AA502540.

SEQ ID NO: 6313 represents the cDNA sequence for clone AA502541.

SEQ ID NO: 6314 represents the cDNA sequence for clone AA502542.

SEQ ID NO: 6315 represents the cDNA sequence for clone AA502543.

SEQ ID NO: 6316 represents the cDNA sequence for clone AA502545.

SEQ ID NO: 6317 represents the cDNA sequence for clone AA502546.

SEQ ID NO: 6318 represents the cDNA sequence for clone AA502655.

SEQ ID NO: 6319 represents the cDNA sequence for clone AA502656.

SEQ ID NO: 6320 represents the cDNA sequence for clone AA502659.

SEQ ID NO: 6321 represents the cDNA sequence for clone AA502660.

SEQ ID NO: 6322 represents the cDNA sequence for clone AA502663.

SEQ ID NO: 6323 represents the cDNA sequence for clone AA502665.

SEQ ID NO: 6324 represents the cDNA sequence for clone AA502668.

SEQ ID NO: 6325 represents the cDNA sequence for clone AA502669.

SEQ ID NO: 6326 represents the cDNA sequence for clone AA502670.

SEQ ID NO: 6327 represents the cDNA sequence for clone AA502674.

SEQ ID NO: 6328 represents the cDNA sequence for clone AA502677.

SEQ ID NO: 6329 represents the cDNA sequence for clone AA502679.

SEQ ID NO: 6330 represents the cDNA sequence for clone AA502681.

SEQ ID NO: 6331 represents the cDNA sequence for clone AA502682.

SEQ ID NO: 6332 represents the cDNA sequence for clone AA502800.

SEQ ID NO: 6333 represents the cDNA sequence for clone AA502803.

SEQ ID NO: 6334 represents the cDNA sequence for clone AA502804.

SEQ ID NO: 6335 represents the cDNA sequence for clone AA502805.

SEQ ID NO: 6336 represents the cDNA sequence for clone AA502809.

SEQ ID NO: 6337 represents the cDNA sequence for clone AA502810.

SEQ ID NO: 6338 represents the cDNA sequence for clone AA502811.

SEQ ID NO: 6339 represents the cDNA sequence for clone AA502813.

SEQ ID NO: 6340 represents the cDNA sequence for clone AA502816.

SEQ ID NO: 6341 represents the cDNA sequence for clone AA502817.

SEQ ID NO: 6342 represents the cDNA sequence for clone AA502819.

SEQ ID NO: 6343 represents the cDNA sequence for clone AA502821.

SEQ ID NO: 6344 represents the cDNA sequence for clone AA502823.

SEQ ID NO: 6345 represents the cDNA sequence for clone AA502828.

SEQ ID NO: 6346 represents the cDNA sequence for clone AA502834.

SEQ ID NO: 6347 represents the cDNA sequence for clone AA502835.

SEQ ID NO: 6348 represents the cDNA sequence for clone AA502837.

SEQ ID NO: 6349 represents the cDNA sequence for clone AA502838.

SEQ ID NO: 6350 represents the cDNA sequence for clone AA502839.

SEQ ID NO: 6351 represents the cDNA sequence for clone AA502841.

SEQ ID NO: 6352 represents the cDNA sequence for clone AA502842.

SEQ ID NO: 6353 represents the cDNA sequence for clone AA502984.

SEQ ID NO: 6354 represents the cDNA sequence for clone AA502985.

SEQ ID NO: 6355 represents the cDNA sequence for clone AA502986.

SEQ ID NO: 6356 represents the cDNA sequence for clone AA502987.

SEQ ID NO: 6357 represents the cDNA sequence for clone AA502988.

SEQ ID NO: 6358 represents the cDNA sequence for clone AA502989.

SEQ ID NO: 6359 represents the cDNA sequence for clone AA502993.

SEQ ID NO: 6360 represents the cDNA sequence for clone AA502996.

SEQ ID NO: 6361 represents the cDNA sequence for clone AA502997.

SEQ ID NO: 6362 represents the cDNA sequence for clone AA502998.

SEQ ID NO: 6363 represents the cDNA sequence for clone AA502999.

SEQ ID NO: 6364 represents the cDNA sequence for clone AA503001.

SEQ ID NO: 6365 represents the cDNA sequence for clone AA503002.

SEQ ID NO: 6366 represents the cDNA sequence for clone AA503005.

SEQ ID NO: 6367 represents the cDNA sequence for clone AA503007.

SEQ ID NO: 6368 represents the cDNA sequence for clone AA503008.

SEQ ID NO: 6369 represents the cDNA sequence for clone AA503009.

SEQ ID NO: 6370 represents the cDNA sequence for clone AA503010.

SEQ ID NO: 6371 represents the cDNA sequence for clone AA503011.

SEQ ID NO: 6372 represents the cDNA sequence for clone AA503013.

SEQ ID NO: 6373 represents the cDNA sequence for clone AA503015.

SEQ ID NO: 6374 represents the cDNA sequence for clone AA503016.

SEQ ID NO: 6375 represents the cDNA sequence for clone AA503017.

SEQ ID NO: 6376 represents the cDNA sequence for clone AA503019.

SEQ ID NO: 6377 represents the cDNA sequence for clone AA503020.

SEQ ID NO: 6378 represents the cDNA sequence for clone AA503021.

SEQ ID NO: 6379 represents the cDNA sequence for clone AA503023.

SEQ ID NO: 6380 represents the cDNA sequence for clone AA503024.

SEQ ID NO: 6381 represents the cDNA sequence for clone AA503025.

SEQ ID NO: 6382 represents the cDNA sequence for clone AA503144.

SEQ ID NO: 6383 represents the cDNA sequence for clone AA503147.

SEQ ID NO: 6384 represents the cDNA sequence for clone AA503149.

SEQ ID NO: 6385 represents the cDNA sequence for clone AA503150.

SEQ ID NO: 6386 represents the cDNA sequence for clone AA503152.

SEQ ID NO: 6387 represents the cDNA sequence for clone AA503156.

SEQ ID NO: 6388 represents the cDNA sequence for clone AA503158.

SEQ ID NO: 6389 represents the cDNA sequence for clone AA503159.

SEQ ID NO: 6390 represents the cDNA sequence for clone AA503160.

SEQ ID NO: 6391 represents the cDNA sequence for clone AA503163.

SEQ ID NO: 6392 represents the cDNA sequence for clone AA503164.

SEQ ID NO: 6393 represents the cDNA sequence for clone AA503165.

SEQ ID NO: 6394 represents the cDNA sequence for clone AA503166.

SEQ ID NO: 6395 represents the cDNA sequence for clone AA503168.

SEQ ID NO: 6396 represents the cDNA sequence for clone AA503171.

SEQ ID NO: 6397 represents the cDNA sequence for clone AA503285.

SEQ ID NO: 6398 represents the cDNA sequence for clone AA503286.

SEQ ID NO: 6399 represents the cDNA sequence for clone AA503289.

SEQ ID NO: 6400 represents the cDNA sequence for clone AA503292.

SEQ ID NO: 6401 represents the cDNA sequence for clone AA503293.

SEQ ID NO: 6402 represents the cDNA sequence for clone AA503294.

SEQ ID NO: 6403 represents the cDNA sequence for clone AA503295.

SEQ ID NO: 6404 represents the cDNA sequence for clone AA503297.

SEQ ID NO: 6405 represents the cDNA sequence for clone AA503300.

SEQ ID NO: 6406 represents the cDNA sequence for clone AA503301.

SEQ ID NO: 6407 represents the cDNA sequence for clone AA503305.

SEQ ID NO: 6408 represents the cDNA sequence for clone AA503309.

SEQ ID NO: 6409 represents the cDNA sequence for clone AA503312.

SEQ ID NO: 6410 represents the cDNA sequence for clone AA503317.

SEQ ID NO: 6411 represents the cDNA sequence for clone AA503318.

SEQ ID NO: 6412 represents the cDNA sequence for clone AA503320.

SEQ ID NO: 6413 represents the cDNA sequence for clone AA503323.

SEQ ID NO: 6414 represents the cDNA sequence for clone AA503324.

SEQ ID NO: 6415 represents the cDNA sequence for clone AA503435.

SEQ ID NO: 6416 represents the cDNA sequence for clone AA503436.

SEQ ID NO: 6417 represents the cDNA sequence for clone AA503437.

SEQ ID NO: 6418 represents the cDNA sequence for clone AA503439.

SEQ ID NO: 6419 represents the cDNA sequence for clone AA503441.

SEQ ID NO: 6420 represents the cDNA sequence for clone AA503442.

SEQ ID NO: 6421 represents the cDNA sequence for clone AA503443.

SEQ ID NO: 6422 represents the cDNA sequence for clone AA503445.

SEQ ID NO: 6423 represents the cDNA sequence for clone AA503446.

SEQ ID NO: 6424 represents the cDNA sequence for clone AA503447.

SEQ ID NO: 6425 represents the cDNA sequence for clone AA503448.

SEQ ID NO: 6426 represents the cDNA sequence for clone AA503449.

SEQ ID NO: 6427 represents the cDNA sequence for clone AA503452.

SEQ ID NO: 6428 represents the cDNA sequence for clone AA503453.

SEQ ID NO: 6429 represents the cDNA sequence for clone AA503455.

SEQ ID NO: 6430 represents the cDNA sequence for clone AA503456.

SEQ ID NO: 6431 represents the cDNA sequence for clone AA503463.

SEQ ID NO: 6432 represents the cDNA sequence for clone AA503464.

SEQ ID NO: 6433 represents the cDNA sequence for clone AA503465.

SEQ ID NO: 6434 represents the cDNA sequence for clone AA503467.

SEQ ID NO: 6435 represents the cDNA sequence for clone AA503468.

SEQ ID NO: 6436 represents the cDNA sequence for clone AA503470.

SEQ ID NO: 6437 represents the cDNA sequence for clone AA503472.

SEQ ID NO: 6438 represents the cDNA sequence for clone AA503473.

SEQ ID NO: 6439 represents the cDNA sequence for clone AA523679.

SEQ ID NO: 6440 represents the cDNA sequence for clone AA523680.

SEQ ID NO: 6441 represents the cDNA sequence for clone AA523681.

SEQ ID NO: 6442 represents the cDNA sequence for clone AA523682.

SEQ ID NO: 6443 represents the cDNA sequence for clone AA523683.

SEQ ID NO: 6444 represents the cDNA sequence for clone AA523684.

SEQ ID NO: 6445 represents the cDNA sequence for clone AA523685.

SEQ ID NO: 6446 represents the cDNA sequence for clone AA523690.

SEQ ID NO: 6447 represents the cDNA sequence for clone AA523696.

SEQ ID NO: 6448 represents the cDNA sequence for clone AA523702.

SEQ ID NO: 6449 represents the cDNA sequence for clone AA523703.

SEQ ID NO: 6450 represents the cDNA sequence for clone AA523707.

SEQ ID NO: 6451 represents the cDNA sequence for clone AA523708.

SEQ ID NO: 6452 represents the cDNA sequence for clone AA523710.

SEQ ID NO: 6453 represents the cDNA sequence for clone AA523711.

SEQ ID NO: 6454 represents the cDNA sequence for clone AA523712.

SEQ ID NO: 6455 represents the cDNA sequence for clone AA523714.

SEQ ID NO: 6456 represents the cDNA sequence for clone AA523715.

SEQ ID NO: 6457 represents the cDNA sequence for clone AA523716.

SEQ ID NO: 6458 represents the cDNA sequence for clone AA523719.

SEQ ID NO: 6459 represents the cDNA sequence for clone AA523810.

SEQ ID NO: 6460 represents the cDNA sequence for clone AA523812.

SEQ ID NO: 6461 represents the cDNA sequence for clone AA523813.

SEQ ID NO: 6462 represents the cDNA sequence for clone AA523819.

SEQ ID NO: 6463 represents the cDNA sequence for clone AA523824.

SEQ ID NO: 6464 represents the cDNA sequence for clone AA523826.

SEQ ID NO: 6465 represents the cDNA sequence for clone AA523829.

SEQ ID NO: 6466 represents the cDNA sequence for clone AA523834.

SEQ ID NO: 6467 represents the cDNA sequence for clone AA523836.

SEQ ID NO: 6468 represents the cDNA sequence for clone AA523839.

SEQ ID NO: 6469 represents the cDNA sequence for clone AA523841.

SEQ ID NO: 6470 represents the cDNA sequence for clone AA523843.

SEQ ID NO: 6471 represents the cDNA sequence for clone AA523844.

SEQ ID NO: 6472 represents the cDNA sequence for clone AA523850.

SEQ ID NO: 6473 represents the cDNA sequence for clone AA524804.

SEQ ID NO: 6474 represents the cDNA sequence for clone AA524807.

SEQ ID NO: 6475 represents the cDNA sequence for clone AA524809.

SEQ ID NO: 6476 represents the cDNA sequence for clone AA524810.

SEQ ID NO: 6477 represents the cDNA sequence for clone AA524816.

SEQ ID NO: 6478 represents the cDNA sequence for clone AA524817.

SEQ ID NO: 6479 represents the cDNA sequence for clone AA524818.

SEQ ID NO: 6480 represents the cDNA sequence for clone AA524820.

SEQ ID NO: 6481 represents the cDNA sequence for clone AA524821.

SEQ ID NO: 6482 represents the cDNA sequence for clone AA524826.

SEQ ID NO: 6483 represents the cDNA sequence for clone AA524828.

SEQ ID NO: 6484 represents the cDNA sequence for clone AA524829.

SEQ ID NO: 6485 represents the cDNA sequence for clone AA524830.

SEQ ID NO: 6486 represents the cDNA sequence for clone AA524832.

SEQ ID NO: 6487 represents the cDNA sequence for clone AA524834.

SEQ ID NO: 6488 represents the cDNA sequence for clone AA524835.

SEQ ID NO: 6489 represents the cDNA sequence for clone AA524837.

SEQ ID NO: 6490 represents the cDNA sequence for clone AA524838.

SEQ ID NO: 6491 represents the cDNA sequence for clone AA524840.

SEQ ID NO: 6492 represents the cDNA sequence for clone AA524842.

SEQ ID NO: 6493 represents the cDNA sequence for clone AA524843.

SEQ ID NO: 6494 represents the cDNA sequence for clone AA525151.

SEQ ID NO: 6495 represents the cDNA sequence for clone AA525153.

SEQ ID NO: 6496 represents the cDNA sequence for clone AA525157.

SEQ ID NO: 6497 represents the cDNA sequence for clone AA525158.

SEQ ID NO: 6498 represents the cDNA sequence for clone AA525160.

SEQ ID NO: 6499 represents the cDNA sequence for clone AA525161.

SEQ ID NO: 6500 represents the cDNA sequence for clone AA525162.

SEQ ID NO: 6501 represents the cDNA sequence for clone AA525165.

SEQ ID NO: 6502 represents the cDNA sequence for clone AA525166.

SEQ ID NO: 6503 represents the cDNA sequence for clone AA525168.

SEQ ID NO: 6504 represents the cDNA sequence for clone AA525169.

SEQ ID NO: 6505 represents the cDNA sequence for clone AA525172.

SEQ ID NO: 6506 represents the cDNA sequence for clone AA525175.

SEQ ID NO: 6507 represents the cDNA sequence for clone AA525176.

SEQ ID NO: 6508 represents the cDNA sequence for clone AA525178.

SEQ ID NO: 6509 represents the cDNA sequence for clone AA525186.

SEQ ID NO: 6510 represents the cDNA sequence for clone AA525187.

SEQ ID NO: 6511 represents the cDNA sequence for clone AA525188.

SEQ ID NO: 6512 represents the cDNA sequence for clone AA525190.

SEQ ID NO: 6513 represents the cDNA sequence for clone AA525192.

SEQ ID NO: 6514 represents the cDNA sequence for clone AA525193.

SEQ ID NO: 6515 represents the cDNA sequence for clone AA525195.

SEQ ID NO: 6516 represents the cDNA sequence for clone AA525201.

SEQ ID NO: 6517 represents the cDNA sequence for clone AA525203.

SEQ ID NO: 6518 represents the cDNA sequence for clone AA525205.

SEQ ID NO: 6519 represents the cDNA sequence for clone AA525207.

SEQ ID NO: 6520 represents the cDNA sequence for clone AA525208.

SEQ ID NO: 6521 represents the cDNA sequence for clone AA525209.

SEQ ID NO: 6522 represents the cDNA sequence for clone AA525213.

SEQ ID NO: 6523 represents the cDNA sequence for clone AA525216.

SEQ ID NO: 6524 represents the cDNA sequence for clone AA525218.

SEQ ID NO: 6525 represents the cDNA sequence for clone AA525219.

SEQ ID NO: 6526 represents the cDNA sequence for clone AA525221.

SEQ ID NO: 6527 represents the cDNA sequence for clone AA525225.

SEQ ID NO: 6528 represents the cDNA sequence for clone AA525226.

SEQ ID NO: 6529 represents the cDNA sequence for clone AA525229.

SEQ ID NO: 6530 represents the cDNA sequence for clone AA525234.

SEQ ID NO: 6531 represents the cDNA sequence for clone AA525236.

SEQ ID NO: 6532 represents the cDNA sequence for clone AA525237.

SEQ ID NO: 6533 represents the cDNA sequence for clone AA525238.

SEQ ID NO: 6534 represents the cDNA sequence for clone AA525239.

SEQ ID NO: 6535 represents the cDNA sequence for clone AA525241.

SEQ ID NO: 6536 represents the cDNA sequence for clone AA525245.

SEQ ID NO: 6537 represents the cDNA sequence for clone AA525246.

SEQ ID NO: 6538 represents the cDNA sequence for clone AA525248.

SEQ ID NO: 6539 represents the cDNA sequence for clone AA525249.

SEQ ID NO: 6540 represents the cDNA sequence for clone AA525279.

SEQ ID NO: 6541 represents the cDNA sequence for clone AA525284.

SEQ ID NO: 6542 represents the cDNA sequence for clone AA525285.

SEQ ID NO: 6543 represents the cDNA sequence for clone AA525286.

SEQ ID NO: 6544 represents the cDNA sequence for clone AA525291.

SEQ ID NO: 6545 represents the cDNA sequence for clone AA525294.

SEQ ID NO: 6546 represents the cDNA sequence for clone AA525296.

SEQ ID NO: 6547 represents the cDNA sequence for clone AA525297.

SEQ ID NO: 6548 represents the cDNA sequence for clone AA525299.

SEQ ID NO: 6549 represents the cDNA sequence for clone AA525300.

SEQ ID NO: 6550 represents the cDNA sequence for clone AA525302.

SEQ ID NO: 6551 represents the cDNA sequence for clone AA525303.

SEQ ID NO: 6552 represents the cDNA sequence for clone AA525304.

SEQ ID NO: 6553 represents the cDNA sequence for clone AA525307.

SEQ ID NO: 6554 represents the cDNA sequence for clone AA525309.

SEQ ID NO: 6555 represents the cDNA sequence for clone AA525310.

SEQ ID NO: 6556 represents the cDNA sequence for clone AA525312.

SEQ ID NO: 6557 represents the cDNA sequence for clone AA525315.

SEQ ID NO: 6558 represents the cDNA sequence for clone AA525317.

SEQ ID NO: 6559 represents the cDNA sequence for clone AA525318.

SEQ ID NO: 6560 represents the cDNA sequence for clone AA525319.

SEQ ID NO: 6561 represents the cDNA sequence for clone AA525320.

SEQ ID NO: 6562 represents the cDNA sequence for clone AA525323.

SEQ ID NO: 6563 represents the cDNA sequence for clone AA525324.

SEQ ID NO: 6564 represents the cDNA sequence for clone AA525326.

SEQ ID NO: 6565 represents the cDNA sequence for clone AA525327.

SEQ ID NO: 6566 represents the cDNA sequence for clone AA525331.

SEQ ID NO: 6567 represents the cDNA sequence for clone AA525334.

SEQ ID NO: 6568 represents the cDNA sequence for clone AA525340.

SEQ ID NO: 6569 represents the cDNA sequence for clone AA525342.

SEQ ID NO: 6570 represents the cDNA sequence for clone AA525343.

SEQ ID NO: 6571 represents the cDNA sequence for clone AA525346.

SEQ ID NO: 6572 represents the cDNA sequence for clone AA525347.

SEQ ID NO: 6573 represents the cDNA sequence for clone AA525383.

SEQ ID NO: 6574 represents the cDNA sequence for clone AA525385.

SEQ ID NO: 6575 represents the cDNA sequence for clone AA525386.

SEQ ID NO: 6576 represents the cDNA sequence for clone AA525389.

SEQ ID NO: 6577 represents the cDNA sequence for clone AA525390.

SEQ ID NO: 6578 represents the cDNA sequence for clone AA525391.

SEQ ID NO: 6579 represents the cDNA sequence for clone AA525392.

SEQ ID NO: 6580 represents the cDNA sequence for clone AA525393.

SEQ ID NO: 6581 represents the cDNA sequence for clone AA525394.

SEQ ID NO: 6582 represents the cDNA sequence for clone AA525395.

SEQ ID NO: 6583 represents the cDNA sequence for clone AA525397.

SEQ ID NO: 6584 represents the cDNA sequence for clone AA525398.

SEQ ID NO: 6585 represents the cDNA sequence for clone AA525400.

SEQ ID NO: 6586 represents the cDNA sequence for clone AA525401.

SEQ ID NO: 6587 represents the cDNA sequence for clone AA525405.

SEQ ID NO: 6588 represents the cDNA sequence for clone AA525406.

SEQ ID NO: 6589 represents the cDNA sequence for clone AA525409.

SEQ ID NO: 6590 represents the cDNA sequence for clone AA525410.

SEQ ID NO: 6591 represents the cDNA sequence for clone AA525412.

SEQ ID NO: 6592 represents the cDNA sequence for clone AA525413.

SEQ ID NO: 6593 represents the cDNA sequence for clone AA525414.

SEQ ID NO: 6594 represents the cDNA sequence for clone AA525416.

SEQ ID NO: 6595 represents the cDNA sequence for clone AA525417.

SEQ ID NO: 6596 represents the cDNA sequence for clone AA525420.

SEQ ID NO: 6597 represents the cDNA sequence for clone AA525424.

SEQ ID NO: 6598 represents the cDNA sequence for clone AA525425.

SEQ ID NO: 6599 represents the cDNA sequence for clone AA525426.

SEQ ID NO: 6600 represents the cDNA sequence for clone AA525427.

SEQ ID NO: 6601 represents the cDNA sequence for clone AA525428.

SEQ ID NO: 6602 represents the cDNA sequence for clone AA525429.

SEQ ID NO: 6603 represents the cDNA sequence for clone AA525431.

SEQ ID NO: 6604 represents the cDNA sequence for clone AA525432.

SEQ ID NO: 6605 represents the cDNA sequence for clone AA525433.

SEQ ID NO: 6606 represents the cDNA sequence for clone AA525438.

SEQ ID NO: 6607 represents the cDNA sequence for clone AA525752.

SEQ ID NO: 6608 represents the cDNA sequence for clone AA525755.

SEQ ID NO: 6609 represents the cDNA sequence for clone AA525756.

SEQ ID NO: 6610 represents the cDNA sequence for clone AA525757.

SEQ ID NO: 6611 represents the cDNA sequence for clone AA525758.

SEQ ID NO: 6612 represents the cDNA sequence for clone AA525759.

SEQ ID NO: 6613 represents the cDNA sequence for clone AA525761.

SEQ ID NO: 6614 represents the cDNA sequence for clone AA525763.

SEQ ID NO: 6615 represents the cDNA sequence for clone AA525764.

SEQ ID NO: 6616 represents the cDNA sequence for clone AA525767.

SEQ ID NO: 6617 represents the cDNA sequence for clone AA525769.

SEQ ID NO: 6618 represents the cDNA sequence for clone AA525770.

SEQ ID NO: 6619 represents the cDNA sequence for clone AA525771.

SEQ ID NO: 6620 represents the cDNA sequence for clone AA525774.

SEQ ID NO: 6621 represents the cDNA sequence for clone AA525775.

SEQ ID NO: 6622 represents the cDNA sequence for clone AA525779.

SEQ ID NO: 6623 represents the cDNA sequence for clone AA525781.

SEQ ID NO: 6624 represents the cDNA sequence for clone AA525782.

SEQ ID NO: 6625 represents the cDNA sequence for clone AA525791.

SEQ ID NO: 6626 represents the cDNA sequence for clone AA525792.

SEQ ID NO: 6627 represents the cDNA sequence for clone AA525793.

SEQ ID NO: 6628 represents the cDNA sequence for clone AA525794.

SEQ ID NO: 6629 represents the cDNA sequence for clone AA525795.

SEQ ID NO: 6630 represents the cDNA sequence for clone AA525800.

SEQ ID NO: 6631 represents the cDNA sequence for clone AA525801.

SEQ ID NO: 6632 represents the cDNA sequence for clone AA525804.

SEQ ID NO: 6633 represents the cDNA sequence for clone AA525805.

SEQ ID NO: 6634 represents the cDNA sequence for clone AA525807.

SEQ ID NO: 6635 represents the cDNA sequence for clone AA525808.

SEQ ID NO: 6636 represents the cDNA sequence for clone AA525809.

SEQ ID NO: 6637 represents the cDNA sequence for clone AA525812.

SEQ ID NO: 6638 represents the cDNA sequence for clone AA525815.

SEQ ID NO: 6639 represents the cDNA sequence for clone AA525816.

SEQ ID NO: 6640 represents the cDNA sequence for clone AA525817.

SEQ ID NO: 6641 represents the cDNA sequence for clone AA525818.

SEQ ID NO: 6642 represents the cDNA sequence for clone AA525823.

SEQ ID NO: 6643 represents the cDNA sequence for clone AA525825.

SEQ ID NO: 6644 represents the cDNA sequence for clone AA525826.

SEQ ID NO: 6645 represents the cDNA sequence for clone AA525828.

SEQ ID NO: 6646 represents the cDNA sequence for clone AA525829.

SEQ ID NO: 6647 represents the cDNA sequence for clone AA525870.

SEQ ID NO: 6648 represents the cDNA sequence for clone AA525873.

SEQ ID NO: 6649 represents the cDNA sequence for clone AA525874.

SEQ ID NO: 6650 represents the cDNA sequence for clone AA525876.

SEQ ID NO: 6651 represents the cDNA sequence for clone AA525879.

SEQ ID NO: 6652 represents the cDNA sequence for clone AA525880.

SEQ ID NO: 6653 represents the cDNA sequence for clone AA525881.

SEQ ID NO: 6654 represents the cDNA sequence for clone AA525883.

SEQ ID NO: 6655 represents the cDNA sequence for clone AA525885.

SEQ ID NO: 6656 represents the cDNA sequence for clone AA525886.

SEQ ID NO: 6657 represents the cDNA sequence for clone AA525888.

SEQ ID NO: 6658 represents the cDNA sequence for clone AA525892.

SEQ ID NO: 6659 represents the cDNA sequence for clone AA525893.

SEQ ID NO: 6660 represents the cDNA sequence for clone AA525894.

SEQ ID NO: 6661 represents the cDNA sequence for clone AA525899.

SEQ ID NO: 6662 represents the cDNA sequence for clone AA525900.

SEQ ID NO: 6663 represents the cDNA sequence for clone AA525903.

SEQ ID NO: 6664 represents the cDNA sequence for clone AA525907.

SEQ ID NO: 6665 represents the cDNA sequence for clone AA525908.

SEQ ID NO: 6666 represents the cDNA sequence for clone AA525910.

SEQ ID NO: 6667 represents the cDNA sequence for clone AA525912.

SEQ ID NO: 6668 represents the cDNA sequence for clone AA525916.

SEQ ID NO: 6669 represents the cDNA sequence for clone AA525918.

SEQ ID NO: 6670 represents the cDNA sequence for clone AA525919.

SEQ ID NO: 6671 represents the cDNA sequence for clone AA525924.

SEQ ID NO: 6672 represents the cDNA sequence for clone AA525926.

SEQ ID NO: 6673 represents the cDNA sequence for clone AA525927.

SEQ ID NO: 6674 represents the cDNA sequence for clone AA525928.

SEQ ID NO: 6675 represents the cDNA sequence for clone AA525929.

SEQ ID NO: 6676 represents the cDNA sequence for clone AA525930.

SEQ ID NO: 6677 represents the cDNA sequence for clone AA525932.

SEQ ID NO: 6678 represents the cDNA sequence for clone AA525934.

SEQ ID NO: 6679 represents the cDNA sequence for clone AA525937.

SEQ ID NO: 6680 represents the cDNA sequence for clone AA525939.

SEQ ID NO: 6681 represents the cDNA sequence for clone AA525940.

SEQ ID NO: 6682 represents the cDNA sequence for clone AA525942.

SEQ ID NO: 6683 represents the cDNA sequence for clone AA525944.

SEQ ID NO: 6684 represents the cDNA sequence for clone AA525945.

SEQ ID NO: 6685 represents the cDNA sequence for clone AA525946.

SEQ ID NO: 6686 represents the cDNA sequence for clone AA525983.

SEQ ID NO: 6687 represents the cDNA sequence for clone AA525985.

SEQ ID NO: 6688 represents the cDNA sequence for clone AA525987.

SEQ ID NO: 6689 represents the cDNA sequence for clone AA525989.

SEQ ID NO: 6690 represents the cDNA sequence for clone AA525990.

SEQ ID NO: 6691 represents the cDNA sequence for clone AA525994.

SEQ ID NO: 6692 represents the cDNA sequence for clone AA525996.

SEQ ID NO: 6693 represents the cDNA sequence for clone AA525998.

SEQ ID NO: 6694 represents the cDNA sequence for clone AA525999.

SEQ ID NO: 6695 represents the cDNA sequence for clone AA526001.

SEQ ID NO: 6696 represents the cDNA sequence for clone AA526005.

SEQ ID NO: 6697 represents the cDNA sequence for clone AA526008.

SEQ ID NO: 6698 represents the cDNA sequence for clone AA526011.

SEQ ID NO: 6699 represents the cDNA sequence for clone AA526012.

SEQ ID NO: 6700 represents the cDNA sequence for clone AA526015.

SEQ ID NO: 6701 represents the cDNA sequence for clone AA526017.

SEQ ID NO: 6702 represents the cDNA sequence for clone AA526018.

SEQ ID NO: 6703 represents the cDNA sequence for clone AA526019.

SEQ ID NO: 6704 represents the cDNA sequence for clone AA526020.

SEQ ID NO: 6705 represents the cDNA sequence for clone AA526087.

SEQ ID NO: 6706 represents the cDNA sequence for clone AA526092.

SEQ ID NO: 6707 represents the cDNA sequence for clone AA526095.

SEQ ID NO: 6708 represents the cDNA sequence for clone AA526098.

SEQ ID NO: 6709 represents the cDNA sequence for clone AA526101.

SEQ ID NO: 6710 represents the cDNA sequence for clone AA526107.

SEQ ID NO: 6711 represents the cDNA sequence for clone AA526108.

SEQ ID NO: 6712 represents the cDNA sequence for clone AA526110.

SEQ ID NO: 6713 represents the cDNA sequence for clone AA526111.

SEQ ID NO: 6714 represents the cDNA sequence for clone AA526114.

SEQ ID NO: 6715 represents the cDNA sequence for clone AA526118.

SEQ ID NO: 6716 represents the cDNA sequence for clone AA526119.

SEQ ID NO: 6717 represents the cDNA sequence for clone AA526121.

SEQ ID NO: 6718 represents the cDNA sequence for clone AA526124.

SEQ ID NO: 6719 represents the cDNA sequence for clone AA526125.

SEQ ID NO: 6720 represents the cDNA sequence for clone AA526127.

SEQ ID NO: 6721 represents the cDNA sequence for clone AA526129.

SEQ ID NO: 6722 represents the cDNA sequence for clone AA526130.

SEQ ID NO: 6723 represents the cDNA sequence for clone AA526192.

SEQ ID NO: 6724 represents the cDNA sequence for clone AA526194.

SEQ ID NO: 6725 represents the cDNA sequence for clone AA526195.

SEQ ID NO: 6726 represents the cDNA sequence for clone AA526196.

SEQ ID NO: 6727 represents the cDNA sequence for clone AA526198.

SEQ ID NO: 6728 represents the cDNA sequence for clone AA526201.

SEQ ID NO: 6729 represents the cDNA sequence for clone AA526203.

SEQ ID NO: 6730 represents the cDNA sequence for clone AA526204.

SEQ ID NO: 6731 represents the cDNA sequence for clone AA526205.

SEQ ID NO: 6732 represents the cDNA sequence for clone AA526206.

SEQ ID NO: 6733 represents the cDNA sequence for clone AA526209.

SEQ ID NO: 6734 represents the cDNA sequence for clone AA526210.

SEQ ID NO: 6735 represents the cDNA sequence for clone AA526213.

SEQ ID NO: 6736 represents the cDNA sequence for clone AA526215.

SEQ ID NO: 6737 represents the cDNA sequence for clone AA526216.

SEQ ID NO: 6738 represents the cDNA sequence for clone AA526220.

SEQ ID NO: 6739 represents the cDNA sequence for clone AA526222.

SEQ ID NO: 6740 represents the cDNA sequence for clone AA526223.

SEQ ID NO: 6741 represents the cDNA sequence for clone AA526225.

SEQ ID NO: 6742 represents the cDNA sequence for clone AA526228.

SEQ ID NO: 6743 represents the cDNA sequence for clone AA526229.

SEQ ID NO: 6744 represents the cDNA sequence for clone AA526301.

SEQ ID NO: 6745 represents the cDNA sequence for clone AA526302.

SEQ ID NO: 6746 represents the cDNA sequence for clone AA526303.

SEQ ID NO: 6747 represents the cDNA sequence for clone AA526305.

SEQ ID NO: 6748 represents the cDNA sequence for clone AA526307.

SEQ ID NO: 6749 represents the cDNA sequence for clone AA526308.

SEQ ID NO: 6750 represents the cDNA sequence for clone AA526309.

SEQ ID NO: 6751 represents the cDNA sequence for clone AA526310.

SEQ ID NO: 6752 represents the cDNA sequence for clone AA526311.

SEQ ID NO: 6753 represents the cDNA sequence for clone AA526314.

SEQ ID NO: 6754 represents the cDNA sequence for clone AA526315.

SEQ ID NO: 6755 represents the cDNA sequence for clone AA526316.

SEQ ID NO: 6756 represents the cDNA sequence for clone AA526317.

SEQ ID NO: 6757 represents the cDNA sequence for clone AA526318.

SEQ ID NO: 6758 represents the cDNA sequence for clone AA526321.

SEQ ID NO: 6759 represents the cDNA sequence for clone AA526325.

SEQ ID NO: 6760 represents the cDNA sequence for clone AA526329.

SEQ ID NO: 6761 represents the cDNA sequence for clone AA526331.

SEQ ID NO: 6762 represents the cDNA sequence for clone AA526334.

SEQ ID NO: 6763 represents the cDNA sequence for clone AA526336.

SEQ ID NO: 6764 represents the cDNA sequence for clone AA526337.

SEQ ID NO: 6765 represents the cDNA sequence for clone AA526342.

SEQ ID NO: 6766 represents the cDNA sequence for clone AA526401.

SEQ ID NO: 6767 represents the cDNA sequence for clone AA526403.

SEQ ID NO: 6768 represents the cDNA sequence for clone AA526404.

SEQ ID NO: 6769 represents the cDNA sequence for clone AA526405.

SEQ ID NO: 6770 represents the cDNA sequence for clone AA526408.

SEQ ID NO: 6771 represents the cDNA sequence for clone AA526409.

SEQ ID NO: 6772 represents the cDNA sequence for clone AA526412.

SEQ ID NO: 6773 represents the cDNA sequence for clone AA526414.

SEQ ID NO: 6774 represents the cDNA sequence for clone AA526415.

SEQ ID NO: 6775 represents the cDNA sequence for clone AA526416.

SEQ ID NO: 6776 represents the cDNA sequence for clone AA526417.

SEQ ID NO: 6777 represents the cDNA sequence for clone AA526418.

SEQ ID NO: 6778 represents the cDNA sequence for clone AA526419.

SEQ ID NO: 6779 represents the cDNA sequence for clone AA526420.

SEQ ID NO: 6780 represents the cDNA sequence for clone AA526421.

SEQ ID NO: 6781 represents the cDNA sequence for clone AA526423.

SEQ ID NO: 6782 represents the cDNA sequence for clone AA526424.

SEQ ID NO: 6783 represents the cDNA sequence for clone AA526428.

SEQ ID NO: 6784 represents the cDNA sequence for clone AA526429.

SEQ ID NO: 6785 represents the cDNA sequence for clone AA526431.

SEQ ID NO: 6786 represents the cDNA sequence for clone AA526434.

SEQ ID NO: 6787 represents the cDNA sequence for clone AA526435.

SEQ ID NO: 6788 represents the cDNA sequence for clone AA526436.

SEQ ID NO: 6789 represents the cDNA sequence for clone AA526437.

SEQ ID NO: 6790 represents the cDNA sequence for clone AA526503.

SEQ ID NO: 6791 represents the cDNA sequence for clone AA526504.

SEQ ID NO: 6792 represents the cDNA sequence for clone AA526506.

SEQ ID NO: 6793 represents the cDNA sequence for clone AA526507.

SEQ ID NO: 6794 represents the cDNA sequence for clone AA526508.

SEQ ID NO: 6795 represents the cDNA sequence for clone AA526509.

SEQ ID NO: 6796 represents the cDNA sequence for clone AA526510.

SEQ ID NO: 6797 represents the cDNA sequence for clone AA526512.

SEQ ID NO: 6798 represents the cDNA sequence for clone AA526513.

SEQ ID NO: 6799 represents the cDNA sequence for clone AA526514.

SEQ ID NO: 6800 represents the cDNA sequence for clone AA526517.

SEQ ID NO: 6801 represents the cDNA sequence for clone AA526519.

SEQ ID NO: 6802 represents the cDNA sequence for clone AA526521.

SEQ ID NO: 6803 represents the cDNA sequence for clone AA526526.

SEQ ID NO: 6804 represents the cDNA sequence for clone AA526527.

SEQ ID NO: 6805 represents the cDNA sequence for clone AA526529.

SEQ ID NO: 6806 represents the cDNA sequence for clone AA526530.

SEQ ID NO: 6807 represents the cDNA sequence for clone AA526531.

SEQ ID NO: 6808 represents the cDNA sequence for clone AA526532.

SEQ ID NO: 6809 represents the cDNA sequence for clone AA526537.

SEQ ID NO: 6810 represents the cDNA sequence for clone AA526538.

SEQ ID NO: 6811 represents the cDNA sequence for clone AA526539.

SEQ ID NO: 6812 represents the cDNA sequence for clone AA526540.

SEQ ID NO: 6813 represents the cDNA sequence for clone AA526542.

SEQ ID NO: 6814 represents the cDNA sequence for clone AA526604.

SEQ ID NO: 6815 represents the cDNA sequence for clone AA526606.

SEQ ID NO: 6816 represents the cDNA sequence for clone AA526607.

SEQ ID NO: 6817 represents the cDNA sequence for clone AA526608.

SEQ ID NO: 6818 represents the cDNA sequence for clone AA526609.

SEQ ID NO: 6819 represents the cDNA sequence for clone AA526610.

SEQ ID NO: 6820 represents the cDNA sequence for clone AA526611.

SEQ ID NO: 6821 represents the cDNA sequence for clone AA526613.

SEQ ID NO: 6822 represents the cDNA sequence for clone AA526616.

SEQ ID NO: 6823 represents the cDNA sequence for clone AA526617.

SEQ ID NO: 6824 represents the cDNA sequence for clone AA526618.

SEQ ID NO: 6825 represents the cDNA sequence for clone AA526621.

SEQ ID NO: 6826 represents the cDNA sequence for clone AA526622.

SEQ ID NO: 6827 represents the cDNA sequence for clone AA526624.

SEQ ID NO: 6828 represents the cDNA sequence for clone AA526625.

SEQ ID NO: 6829 represents the cDNA sequence for clone AA526627.

SEQ ID NO: 6830 represents the cDNA sequence for clone AA526628.

SEQ ID NO: 6831 represents the cDNA sequence for clone AA526632.

SEQ ID NO: 6832 represents the cDNA sequence for clone AA526633.

SEQ ID NO: 6833 represents the cDNA sequence for clone AA526635.

SEQ ID NO: 6834 represents the cDNA sequence for clone AA526637.

SEQ ID NO: 6835 represents the cDNA sequence for clone AA526644.

SEQ ID NO: 6836 represents the cDNA sequence for clone AA526646.

SEQ ID NO: 6837 represents the cDNA sequence for clone AA526647.

SEQ ID NO: 6838 represents the cDNA sequence for clone AA526648.

SEQ ID NO: 6839 represents the cDNA sequence for clone AA526651.

SEQ ID NO: 6840 represents the cDNA sequence for clone AA526653.

SEQ ID NO: 6841 represents the cDNA sequence for clone AA526655.

SEQ ID NO: 6842 represents the cDNA sequence for clone AA526656.

SEQ ID NO: 6843 represents the cDNA sequence for clone AA526657.

SEQ ID NO: 6844 represents the cDNA sequence for clone AA526658.

SEQ ID NO: 6845 represents the cDNA sequence for clone AA526659.

SEQ ID NO: 6846 represents the cDNA sequence for clone AA526661.

SEQ ID NO: 6847 represents the cDNA sequence for clone AA526662.

SEQ ID NO: 6848 represents the cDNA sequence for clone AA526663.

SEQ ID NO: 6849 represents the cDNA sequence for clone AA526747.

SEQ ID NO: 6850 represents the cDNA sequence for clone AA526748.

SEQ ID NO: 6851 represents the cDNA sequence for clone AA526752.

SEQ ID NO: 6852 represents the cDNA sequence for clone AA526753.

SEQ ID NO: 6853 represents the cDNA sequence for clone AA526755.

SEQ ID NO: 6854 represents the cDNA sequence for clone AA526757.

SEQ ID NO: 6855 represents the cDNA sequence for clone AA526764.

SEQ ID NO: 6856 represents the cDNA sequence for clone AA526766.

SEQ ID NO: 6857 represents the cDNA sequence for clone AA526767.

SEQ ID NO: 6858 represents the cDNA sequence for clone AA526768.

SEQ ID NO: 6859 represents the cDNA sequence for clone AA526770.

SEQ ID NO: 6860 represents the cDNA sequence for clone AA526771.

SEQ ID NO: 6861 represents the cDNA sequence for clone AA526774.

SEQ ID NO: 6862 represents the cDNA sequence for clone AA526775.

SEQ ID NO: 6863 represents the cDNA sequence for clone AA526777.

SEQ ID NO: 6864 represents the cDNA sequence for clone AA526778.

SEQ ID NO: 6865 represents the cDNA sequence for clone AA526781.

SEQ ID NO: 6866 represents the cDNA sequence for clone AA526782.

SEQ ID NO: 6867 represents the cDNA sequence for clone AA526786.

SEQ ID NO: 6868 represents the cDNA sequence for clone AA526789.

SEQ ID NO: 6869 represents the cDNA sequence for clone AA526790.

SEQ ID NO: 6870 represents the cDNA sequence for clone AA526792.

SEQ ID NO: 6871 represents the cDNA sequence for clone AA526793.

SEQ ID NO: 6872 represents the cDNA sequence for clone AA526794.

SEQ ID NO: 6873 represents the cDNA sequence for clone AA526795.

SEQ ID NO: 6874 represents the cDNA sequence for clone AA526798.

SEQ ID NO: 6875 represents the cDNA sequence for clone AA526802.

SEQ ID NO: 6876 represents the cDNA sequence for clone AA526803.

SEQ ID NO: 6877 represents the cDNA sequence for clone AA526807.

SEQ ID NO: 6878 represents the cDNA sequence for clone AA526808.

SEQ ID NO: 6879 represents the cDNA sequence for clone AA533561.

SEQ ID NO: 6880 represents the cDNA sequence for clone AA533564.

SEQ ID NO: 6881 represents the cDNA sequence for clone AA533566.

SEQ ID NO: 6882 represents the cDNA sequence for clone AA533574.

SEQ ID NO: 6883 represents the cDNA sequence for clone AA533576.

SEQ ID NO: 6884 represents the cDNA sequence for clone AA542990.

SEQ ID NO: 6885 represents the cDNA sequence for clone AA542991.

SEQ ID NO: 6886 represents the cDNA sequence for clone AA542993.

SEQ ID NO: 6887 represents the cDNA sequence for clone AA542995.

SEQ ID NO: 6888 represents the cDNA sequence for clone AA542999.

SEQ ID NO: 6889 represents the cDNA sequence for clone AA543000.

SEQ ID NO: 6890 represents the cDNA sequence for clone AA543006.

SEQ ID NO: 6891 represents the cDNA sequence for clone AA543008.

SEQ ID NO: 6892 represents the cDNA sequence for clone AA543009.

SEQ ID NO: 6893 represents the cDNA sequence for clone AA543011.

SEQ ID NO: 6894 represents the cDNA sequence for clone AA543013.

SEQ ID NO: 6895 represents the cDNA sequence for clone AA543014.

SEQ ID NO: 6896 represents the cDNA sequence for clone AA543015.

SEQ ID NO: 6897 represents the cDNA sequence for clone AA543022.

SEQ ID NO: 6898 represents the cDNA sequence for clone AA543024.

SEQ ID NO: 6899 represents the cDNA sequence for clone AA543025.

SEQ ID NO: 6900 represents the cDNA sequence for clone AA581078.

SEQ ID NO: 6901 represents the cDNA sequence for clone AA581091.

SEQ ID NO: 6902 represents the cDNA sequence for clone AA581095.

SEQ ID NO: 6903 represents the cDNA sequence for clone AA581098.

SEQ ID NO: 6904 represents the cDNA sequence for clone AA581101.

SEQ ID NO: 6905 represents the cDNA sequence for clone AA581102.

SEQ ID NO: 6906 represents the cDNA sequence for clone AA581104.

SEQ ID NO: 6907 represents the cDNA sequence for clone AA581112.

SEQ ID NO: 6908 represents the cDNA sequence for clone AA581115.

SEQ ID NO: 6909 represents the cDNA sequence for clone AA581120.

SEQ ID NO: 6910 represents the cDNA sequence for clone AA581122.

SEQ ID NO: 6911 represents the cDNA sequence for clone AA581127.

SEQ ID NO: 6912 represents the cDNA sequence for clone AA581129.

SEQ ID NO: 6913 represents the cDNA sequence for clone AA581130.

SEQ ID NO: 6914 represents the cDNA sequence for clone AA581138.

SEQ ID NO: 6915 represents the cDNA sequence for clone AA581141.

SEQ ID NO: 6916 represents the cDNA sequence for clone AA581142.

SEQ ID NO: 6917 represents the cDNA sequence for clone AA581146.

SEQ ID NO: 6918 represents the cDNA sequence for clone AA581147.

SEQ ID NO: 6919 represents the cDNA sequence for clone AA581149.

SEQ ID NO: 6920 represents the cDNA sequence for clone AA581152.

SEQ ID NO: 6921 represents the cDNA sequence for clone AA581154.

SEQ ID NO: 6922 represents the cDNA sequence for clone AA581157.

SEQ ID NO: 6923 represents the cDNA sequence for clone AA581163.

SEQ ID NO: 6924 represents the cDNA sequence for clone AA581165.

SEQ ID NO: 6925 represents the cDNA sequence for clone AA581168.

SEQ ID NO: 6926 represents the cDNA sequence for clone AA581170.

SEQ ID NO: 6927 represents the cDNA sequence for clone AA581463.

SEQ ID NO: 6928 represents the cDNA sequence for clone AA581464.

SEQ ID NO: 6929 represents the cDNA sequence for clone AA581466.

SEQ ID NO: 6930 represents the cDNA sequence for clone AA581469.

SEQ ID NO: 6931 represents the cDNA sequence for clone AA581470.

SEQ ID NO: 6932 represents the cDNA sequence for clone AA581473.

SEQ ID NO: 6933 represents the cDNA sequence for clone AA581474.

SEQ ID NO: 6934 represents the cDNA sequence for clone AA581477.

SEQ ID NO: 6935 represents the cDNA sequence for clone AA581480.

SEQ ID NO: 6936 represents the cDNA sequence for clone AA581483.

SEQ ID NO: 6937 represents the cDNA sequence for clone AA581485.

SEQ ID NO: 6938 represents the cDNA sequence for clone AA581486.

SEQ ID NO: 6939 represents the cDNA sequence for clone AA581490.

SEQ ID NO: 6940 represents the cDNA sequence for clone AA581492.

SEQ ID NO: 6941 represents the cDNA sequence for clone AA581493.

SEQ ID NO: 6942 represents the cDNA sequence for clone AA581494.

SEQ ID NO: 6943 represents the cDNA sequence for clone AA581496.

SEQ ID NO: 6944 represents the cDNA sequence for clone AA581498.

SEQ ID NO: 6945 represents the cDNA sequence for clone AA581500.

SEQ ID NO: 6946 represents the cDNA sequence for clone AA581507.

SEQ ID NO: 6947 represents the cDNA sequence for clone AA581508.

SEQ ID NO: 6948 represents the cDNA sequence for clone AA587597.

SEQ ID NO: 6949 represents the cDNA sequence for clone AA587606.

SEQ ID NO: 6950 represents the cDNA sequence for clone AA586310.

SEQ ID NO: 6951 represents the cDNA sequence for clone AA586313.

SEQ ID NO: 6952 represents the cDNA sequence for clone AA586314.

SEQ ID NO: 6953 represents the cDNA sequence for clone AA586322.

SEQ ID NO: 6954 represents the cDNA sequence for clone AA586326.

SEQ ID NO: 6955 represents the cDNA sequence for clone AA586331.

SEQ ID NO: 6956 represents the cDNA sequence for clone AA586335.

SEQ ID NO: 6957 represents the cDNA sequence for clone AA627933.

SEQ ID NO: 6958 represents the cDNA sequence for clone AA628034.

SEQ ID NO: 6959 represents the cDNA sequence for clone AA628040.

SEQ ID NO: 6960 represents the cDNA sequence for clone AA627230.

SEQ ID NO: 6961 represents the cDNA sequence for clone AA627235.

SEQ ID NO: 6962 represents the cDNA sequence for clone AA627237.

SEQ ID NO: 6963 represents the cDNA sequence for clone AA627241.

SEQ ID NO: 6964 represents the cDNA sequence for clone AA627262.

SEQ ID NO: 6965 represents the cDNA sequence for clone AA627304.

SEQ ID NO: 6966 represents the cDNA sequence for clone AA627313.

SEQ ID NO: 6967 represents the cDNA sequence for clone AA627330.

SEQ ID NO: 6968 represents the cDNA sequence for clone AA627333.

SEQ ID NO: 6969 represents the cDNA sequence for clone AA627335.

SEQ ID NO: 6970 represents the cDNA sequence for clone AA627343.

SEQ ID NO: 6971 represents the cDNA sequence for clone AA629620.

SEQ ID NO: 6972 represents the cDNA sequence for clone AA629629.

SEQ ID NO: 6973 represents the cDNA sequence for clone AA629632.

SEQ ID NO: 6974 represents the cDNA sequence for clone AA629633.

SEQ ID NO: 6975 represents the cDNA sequence for clone AA629636.

SEQ ID NO: 6976 represents the cDNA sequence for clone AA629637.

SEQ ID NO: 6977 represents the cDNA sequence for clone AA629638.

SEQ ID NO: 6978 represents the cDNA sequence for clone AA629640.

SEQ ID NO: 6979 represents the cDNA sequence for clone AA629645.

SEQ ID NO: 6980 represents the cDNA sequence for clone AA633747.

SEQ ID NO: 6981 represents the cDNA sequence for clone AA633751.

SEQ ID NO: 6982 represents the cDNA sequence for clone AA633753.

SEQ ID NO: 6983 represents the cDNA sequence for clone AA633755.

SEQ ID NO: 6984 represents the cDNA sequence for clone AA633758.

SEQ ID NO: 6985 represents the cDNA sequence for clone AA633762.

SEQ ID NO: 6986 represents the cDNA sequence for clone AA633763.

SEQ ID NO: 6987 represents the cDNA sequence for clone AA633766.

SEQ ID NO: 6988 represents the cDNA sequence for clone AA633767.

SEQ ID NO: 6989 represents the cDNA sequence for clone AA633769.

SEQ ID NO: 6990 represents the cDNA sequence for clone AA633772.

SEQ ID NO: 6991 represents the cDNA sequence for clone AA633779.

SEQ ID NO: 6992 represents the cDNA sequence for clone AA633780.

SEQ ID NO: 6993 represents the cDNA sequence for clone AA633781.

SEQ ID NO: 6994 represents the cDNA sequence for clone AA633783.

SEQ ID NO: 6995 represents the cDNA sequence for clone AA642156.

SEQ ID NO: 6996 represents the cDNA sequence for clone AA642166.

SEQ ID NO: 6997 represents the cDNA sequence for clone AA643412.

SEQ ID NO: 6998 represents the cDNA sequence for clone AA643419.

SEQ ID NO: 6999 represents the cDNA sequence for clone AA643427.

SEQ ID NO: 7000 represents the cDNA sequence for clone AA643437.

SEQ ID NO: 7001 represents the cDNA sequence for clone AA643440.

SEQ ID NO: 7002 represents the cDNA sequence for clone AA643441.

SEQ ID NO: 7003 represents the cDNA sequence for clone AA643449.

SEQ ID NO: 7004 represents the cDNA sequence for clone AA643450.

SEQ ID NO: 7005 represents the cDNA sequence for clone AA643451.

SEQ ID NO: 7006 represents the cDNA sequence for clone AA643460.

SEQ ID NO: 7007 represents the cDNA sequence for clone AA643464.

SEQ ID NO: 7008 represents the cDNA sequence for clone AA643470.

SEQ ID NO: 7009 represents the cDNA sequence for clone AA643476.

SEQ ID NO: 7010 represents the cDNA sequence for clone AA643487.

SEQ ID NO: 7011 represents the cDNA sequence for clone AA650077.

SEQ ID NO: 7012 represents the cDNA sequence for clone AA650078.

SEQ ID NO: 7013 represents the cDNA sequence for clone AA650081.

SEQ ID NO: 7014 represents the cDNA sequence for clone AA650084.

SEQ ID NO: 7015 represents the cDNA sequence for clone AA650086.

SEQ ID NO: 7016 represents the cDNA sequence for clone AA650309.

SEQ ID NO: 7017 represents the cDNA sequence for clone AA650312.

SEQ ID NO: 7018 represents the cDNA sequence for clone AA650529.

SEQ ID NO: 7019 represents the cDNA sequence for clone AA650533.

SEQ ID NO: 7020 represents the cDNA sequence for clone AA650572.

SEQ ID NO: 7021 represents the cDNA sequence for clone AA650574.

SEQ ID NO: 7022 represents the cDNA sequence for clone AA650575.

SEQ ID NO: 7023 represents the cDNA sequence for clone AA650614.

SEQ ID NO: 7024 represents the cDNA sequence for clone AA650615.

SEQ ID NO: 7025 represents the cDNA sequence for clone AA650618.

SEQ ID NO: 7026 represents the cDNA sequence for clone AA650620.

SEQ ID NO: 7027 represents the cDNA sequence for clone AA653905.

SEQ ID NO: 7028 represents the cDNA sequence for clone AA653907.

SEQ ID NO: 7029 represents the cDNA sequence for clone AA654085.

SEQ ID NO: 7030 represents the cDNA sequence for clone AA654087.

SEQ ID NO: 7031 represents the cDNA sequence for clone AA658523.

SEQ ID NO: 7032 represents the cDNA sequence for clone AA658528.

SEQ ID NO: 7033 represents the cDNA sequence for clone AA658532.

SEQ ID NO: 7034 represents the cDNA sequence for clone AA658534.

SEQ ID NO: 7035 represents the cDNA sequence for clone AA658573.

SEQ ID NO: 7036 represents the cDNA sequence for clone AA658581.

SEQ ID NO: 7037 represents the cDNA sequence for clone AA658583.

SEQ ID NO: 7038 represents the cDNA sequence for clone AA658584.

SEQ ID NO: 7039 represents the cDNA sequence for clone AA668694.

SEQ ID NO: 7040 represents the cDNA sequence for clone AA668698.

SEQ ID NO: 7041 represents the cDNA sequence for clone AA668702.

SEQ ID NO: 7042 represents the cDNA sequence for clone AA668707.

SEQ ID NO: 7043 represents the cDNA sequence for clone AA668711.

SEQ ID NO: 7044 represents the cDNA sequence for clone AA668715.

SEQ ID NO: 7045 represents the cDNA sequence for clone AA668717.

SEQ ID NO: 7046 represents the cDNA sequence for clone AA668723.

SEQ ID NO: 7047 represents the cDNA sequence for clone AA669218.

SEQ ID NO: 7048 represents the cDNA sequence for clone AA669219.

SEQ ID NO: 7049 represents the cDNA sequence for clone AA669220.

SEQ ID NO: 7050 represents the cDNA sequence for clone AA669222.

SEQ ID NO: 7051 represents the cDNA sequence for clone AA669224.

SEQ ID NO: 7052 represents the cDNA sequence for clone AA669226.

SEQ ID NO: 7053 represents the cDNA sequence for clone AA669228.

SEQ ID NO: 7054 represents the cDNA sequence for clone AA669232.

SEQ ID NO: 7055 represents the cDNA sequence for clone AA669234.

SEQ ID NO: 7056 represents the cDNA sequence for clone AA669235.

SEQ ID NO: 7057 represents the cDNA sequence for clone AA669238.

SEQ ID NO: 7058 represents the cDNA sequence for clone AA669243.

SEQ ID NO: 7059 represents the cDNA sequence for clone AA669244.

SEQ ID NO: 7060 represents the cDNA sequence for clone AA669249.

SEQ ID NO: 7061 represents the cDNA sequence for clone AA669250.

SEQ ID NO: 7062 represents the cDNA sequence for clone AA669253.

SEQ ID NO: 7063 represents the cDNA sequence for clone AA669256.

SEQ ID NO: 7064 represents the cDNA sequence for clone AA669258.

SEQ ID NO: 7065 represents the cDNA sequence for clone AA669264.

SEQ ID NO: 7066 represents the cDNA sequence for clone AA669267.

SEQ ID NO: 7067 represents the cDNA sequence for clone AA669268.

SEQ ID NO: 7068 represents the cDNA sequence for clone AA669269.

SEQ ID NO: 7069 represents the cDNA sequence for clone AA669270.

SEQ ID NO: 7070 represents the cDNA sequence for clone AA669306.

SEQ ID NO: 7071 represents the cDNA sequence for clone AA669309.

SEQ ID NO: 7072 represents the cDNA sequence for clone AA669310.

SEQ ID NO: 7073 represents the cDNA sequence for clone AA669313.

SEQ ID NO: 7074 represents the cDNA sequence for clone AA669315.

SEQ ID NO: 7075 represents the cDNA sequence for clone AA669316.

SEQ ID NO: 7076 represents the cDNA sequence for clone AA669319.

SEQ ID NO: 7077 represents the cDNA sequence for clone AA669320.

SEQ ID NO: 7078 represents the cDNA sequence for clone AA669327.

SEQ ID NO: 7079 represents the cDNA sequence for clone AA669331.

SEQ ID NO: 7080 represents the cDNA sequence for clone AA669410.

SEQ ID NO: 7081 represents the cDNA sequence for clone AA669411.

SEQ ID NO: 7082 represents the cDNA sequence for clone AA669419.

SEQ ID NO: 7083 represents the cDNA sequence for clone AA669421.

SEQ ID NO: 7084 represents the cDNA sequence for clone AA669423.

SEQ ID NO: 7085 represents the cDNA sequence for clone AA669426.

SEQ ID NO: 7086 represents the cDNA sequence for clone AA669427.

SEQ ID NO: 7087 represents the cDNA sequence for clone AA669429.

SEQ ID NO: 7088 represents the cDNA sequence for clone AA669430.

SEQ ID NO: 7089 represents the cDNA sequence for clone AA669539.

SEQ ID NO: 7090 represents the cDNA sequence for clone AA669543.

SEQ ID NO: 7091 represents the cDNA sequence for clone AA669544.

SEQ ID NO: 7092 represents the cDNA sequence for clone AA669547.

SEQ ID NO: 7093 represents the cDNA sequence for clone AA669549.

SEQ ID NO: 7094 represents the cDNA sequence for clone AA669552.

SEQ ID NO: 7095 represents the cDNA sequence for clone AA669558.

SEQ ID NO: 7096 represents the cDNA sequence for clone AA669559.

SEQ ID NO: 7097 represents the cDNA sequence for clone AA669560.

SEQ ID NO: 7098 represents the cDNA sequence for clone AA669564.

SEQ ID NO: 7099 represents the cDNA sequence for clone AA669565.

SEQ ID NO: 7100 represents the cDNA sequence for clone AA669567.

SEQ ID NO: 7101 represents the cDNA sequence for clone AA669569.

SEQ ID NO: 7102 represents the cDNA sequence for clone AA669570.

SEQ ID NO: 7103 represents the cDNA sequence for clone AA669572.

SEQ ID NO: 7104 represents the cDNA sequence for clone AA669574.

SEQ ID NO: 7105 represents the cDNA sequence for clone AA669577.

SEQ ID NO: 7106 represents the cDNA sequence for clone AA669578.

SEQ ID NO: 7107 represents the cDNA sequence for clone AA669580.

SEQ ID NO: 7108 represents the cDNA sequence for clone AA669584.

SEQ ID NO: 7109 represents the cDNA sequence for clone AA669588.

SEQ ID NO: 7110 represents the cDNA sequence for clone AA669590.

SEQ ID NO: 7111 represents the cDNA sequence for clone AA669592.

SEQ ID NO: 7112 represents the cDNA sequence for clone AA669593.

SEQ ID NO: 7113 represents the cDNA sequence for clone AA669600.

SEQ ID NO: 7114 represents the cDNA sequence for clone AA669605.

SEQ ID NO: 7115 represents the cDNA sequence for clone AA669608.

SEQ ID NO: 7116 represents the cDNA sequence for clone AA669611.

SEQ ID NO: 7117 represents the cDNA sequence for clone AA669612.

SEQ ID NO: 7118 represents the cDNA sequence for clone AA669614.

SEQ ID NO: 7119 represents the cDNA sequence for clone AA669616.

SEQ ID NO: 7120 represents the cDNA sequence for clone AA669618.

SEQ ID NO: 7121 represents the cDNA sequence for clone AA669620.

SEQ ID NO: 7122 represents the cDNA sequence for clone AA669627.

SEQ ID NO: 7123 represents the cDNA sequence for clone AA669631.

SEQ ID NO: 7124 represents the cDNA sequence for clone AA669632.

SEQ ID NO: 7125 represents the cDNA sequence for clone AA669634.

SEQ ID NO: 7126 represents the cDNA sequence for clone AA669643.

SEQ ID NO: 7127 represents the cDNA sequence for clone AA669646.

SEQ ID NO: 7128 represents the cDNA sequence for clone AA669648.

SEQ ID NO: 7129 represents the cDNA sequence for clone AA669658.

SEQ ID NO: 7130 represents the cDNA sequence for clone AA669659.

SEQ ID NO: 7131 represents the cDNA sequence for clone AA669663.

SEQ ID NO: 7132 represents the cDNA sequence for clone AA669664.

SEQ ID NO: 7133 represents the cDNA sequence for clone AA669668.

SEQ ID NO: 7134 represents the cDNA sequence for clone AA669669.

SEQ ID NO: 7135 represents the cDNA sequence for clone AA669671.

SEQ ID NO: 7136 represents the cDNA sequence for clone AA669673.

SEQ ID NO: 7137 represents the cDNA sequence for clone AA669677.

SEQ ID NO: 7138 represents the cDNA sequence for clone AA669681.

SEQ ID NO: 7139 represents the cDNA sequence for clone AA669689.

SEQ ID NO: 7140 represents the cDNA sequence for clone AA669690.

SEQ ID NO: 7141 represents the cDNA sequence for clone AA669691.

SEQ ID NO: 7142 represents the cDNA sequence for clone AA669694.

SEQ ID NO: 7143 represents the cDNA sequence for clone AA669695.

SEQ ID NO: 7144 represents the cDNA sequence for clone AA669704.

SEQ ID NO: 7145 represents the cDNA sequence for clone AA669705.

SEQ ID NO: 7146 represents the cDNA sequence for clone AA669706.

SEQ ID NO: 7147 represents the cDNA sequence for clone AF034533.

SEQ ID NO: 7148 represents the cDNA sequence for clone AA680250.

SEQ ID NO: 7149 represents the cDNA sequence for clone AA680252.

SEQ ID NO: 7150 represents the cDNA sequence for clone AA680257.

SEQ ID NO: 7151 represents the cDNA sequence for clone AA680261.

SEQ ID NO: 7152 represents the cDNA sequence for clone AA680262.

SEQ ID NO: 7153 represents the cDNA sequence for clone AA680263.

SEQ ID NO: 7154 represents the cDNA sequence for clone AA680269.

SEQ ID NO: 7155 represents the cDNA sequence for clone AA680307.

SEQ ID NO: 7156 represents the cDNA sequence for clone AA680311.

SEQ ID NO: 7157 represents the cDNA sequence for clone AA680313.

SEQ ID NO: 7158 represents the cDNA sequence for clone AA680314.

SEQ ID NO: 7159 represents the cDNA sequence for clone AA680315.

SEQ ID NO: 7160 represents the cDNA sequence for clone AA680318.

SEQ ID NO: 7161 represents the cDNA sequence for clone AA680319.

SEQ ID NO: 7162 represents the cDNA sequence for clone AA680320.

SEQ ID NO: 7163 represents the cDNA sequence for clone AA680321.

SEQ ID NO: 7164 represents the cDNA sequence for clone AA680326.

SEQ ID NO: 7165 represents the cDNA sequence for clone AA679877.

SEQ ID NO: 7166 represents the cDNA sequence for clone AA679885.

SEQ ID NO: 7167 represents the cDNA sequence for clone AA679886.

SEQ ID NO: 7168 represents the cDNA sequence for clone AA679901.

SEQ ID NO: 7169 represents the cDNA sequence for clone AA679911.

SEQ ID NO: 7170 represents the cDNA sequence for clone AA679922.

SEQ ID NO: 7171 represents the cDNA sequence for clone AA679930.

SEQ ID NO: 7172 represents the cDNA sequence for clone AA679931.

SEQ ID NO: 7173 represents the cDNA sequence for clone AA679933.

SEQ ID NO: 7174 represents the cDNA sequence for clone AA679937.

SEQ ID NO: 7175 represents the cDNA sequence for clone AA679938.

SEQ ID NO: 7176 represents the cDNA sequence for clone AA679939.

SEQ ID NO: 7177 represents the cDNA sequence for clone AA679942.

SEQ ID NO: 7178 represents the cDNA sequence for clone AA679945.

SEQ ID NO: 7179 represents the cDNA sequence for clone AA679946.

SEQ ID NO: 7180 represents the cDNA sequence for clone AA679194.

SEQ ID NO: 7181 represents the cDNA sequence for clone AA679195.

SEQ ID NO: 7182 represents the cDNA sequence for clone AA679196.

SEQ ID NO: 7183 represents the cDNA sequence for clone AA679203.

SEQ ID NO: 7184 represents the cDNA sequence for clone AA679206.

SEQ ID NO: 7185 represents the cDNA sequence for clone AA679207.

SEQ ID NO: 7186 represents the cDNA sequence for clone AA679210.

SEQ ID NO: 7187 represents the cDNA sequence for clone AA679212.

SEQ ID NO: 7188 represents the cDNA sequence for clone AA679254.

SEQ ID NO: 7189 represents the cDNA sequence for clone AA679263.

SEQ ID NO: 7190 represents the cDNA sequence for clone AA679266.

SEQ ID NO: 7191 represents the cDNA sequence for clone AA679268.

SEQ ID NO: 7192 represents the cDNA sequence for clone AA679269.

SEQ ID NO: 7193 represents the cDNA sequence for clone AA679270.

SEQ ID NO: 7194 represents the cDNA sequence for clone AA679273.

SEQ ID NO: 7195 represents the cDNA sequence for clone AA773088.

SEQ ID NO: 7196 represents the cDNA sequence for clone AA773090.

SEQ ID NO: 7197 represents the cDNA sequence for clone AA773093.

SEQ ID NO: 7198 represents the cDNA sequence for clone AA773095.

SEQ ID NO: 7199 represents the cDNA sequence for clone AA773100.

SEQ ID NO: 7200 represents the cDNA sequence for clone AA773101.

SEQ ID NO: 7201 represents the cDNA sequence for clone AA773102.

SEQ ID NO: 7202 represents the cDNA sequence for clone AA773105.

SEQ ID NO: 7203 represents the cDNA sequence for clone AA773107.

SEQ ID NO: 7204 represents the cDNA sequence for clone AA773108.

SEQ ID NO: 7205 represents the cDNA sequence for clone AA773111.

SEQ ID NO: 7206 represents the cDNA sequence for clone AA773116.

SEQ ID NO: 7207 represents the cDNA sequence for clone AA773117.

SEQ ID NO: 7208 represents the cDNA sequence for clone AA773125.

SEQ ID NO: 7209 represents the cDNA sequence for clone AA773132.

SEQ ID NO: 7210 represents the cDNA sequence for clone AA773134.

SEQ ID NO: 7211 represents the cDNA sequence for clone AA773138.

SEQ ID NO: 7212 represents the cDNA sequence for clone AA773139.

SEQ ID NO: 7213 represents the cDNA sequence for clone AA773145.

SEQ ID NO: 7214 represents the cDNA sequence for clone AA773148.

SEQ ID NO: 7215 represents the cDNA sequence for clone AA773156.

SEQ ID NO: 7216 represents the cDNA sequence for clone AA775062.

SEQ ID NO: 7217 represents the cDNA sequence for clone AA775063.

SEQ ID NO: 7218 represents the cDNA sequence for clone AA775073.

SEQ ID NO: 7219 represents the cDNA sequence for clone AA782307.

SEQ ID NO: 7220 represents the cDNA sequence for clone AA782308.

SEQ ID NO: 7221 represents the cDNA sequence for clone AA782311.

SEQ ID NO: 7222 represents the cDNA sequence for clone AA782313.

SEQ ID NO: 7223 represents the cDNA sequence for clone AA782323.

SEQ ID NO: 7224 represents the cDNA sequence for clone AA782328.

SEQ ID NO: 7225 represents the cDNA sequence for clone AA782334.

SEQ ID NO: 7226 represents the cDNA sequence for clone AA782337.

SEQ ID NO: 7227 represents the cDNA sequence for clone AA782340.

SEQ ID NO: 7228 represents the cDNA sequence for clone AA782345.

SEQ ID NO: 7229 represents the cDNA sequence for clone AA782346.

SEQ ID NO: 7230 represents the cDNA sequence for clone AA782352.

SEQ ID NO: 7231 represents the cDNA sequence for clone AA782353.

SEQ ID NO: 7232 represents the cDNA sequence for clone AA782358.

SEQ ID NO: 7233 represents the cDNA sequence for clone AA782359.

SEQ ID NO: 7234 represents the cDNA sequence for clone AA782366.

SEQ ID NO: 7235 represents the cDNA sequence for clone AA782369.

SEQ ID NO: 7236 represents the cDNA sequence for clone AA782370.

SEQ ID NO: 7237 represents the cDNA sequence for clone AA782372.

SEQ ID NO: 7238 represents the cDNA sequence for clone AA782377.

SEQ ID NO: 7239 represents the cDNA sequence for clone AA782378.

SEQ ID NO: 7240 represents the cDNA sequence for clone AA782383.

SEQ ID NO: 7241 represents the cDNA sequence for clone AA782384.

SEQ ID NO: 7242 represents the cDNA sequence for clone AA782873.

SEQ ID NO: 7243 represents the cDNA sequence for clone AA782876.

SEQ ID NO: 7244 represents the cDNA sequence for clone AA782883.

SEQ ID NO: 7245 represents the cDNA sequence for clone AA782886.

SEQ ID NO: 7246 represents the cDNA sequence for clone AA782891.

SEQ ID NO: 7247 represents the cDNA sequence for clone AA782903.

SEQ ID NO: 7248 represents the cDNA sequence for clone AA808800.

SEQ ID NO: 7249 represents the cDNA sequence for clone AA808802.

SEQ ID NO: 7250 represents the cDNA sequence for clone AA808840.

SEQ ID NO: 7251 represents the cDNA sequence for clone AA808841.

SEQ ID NO: 7252 represents the cDNA sequence for clone AA808844.

SEQ ID NO: 7253 represents the cDNA sequence for clone AA808970.

SEQ ID NO: 7254 represents the cDNA sequence for clone AA809001.

SEQ ID NO: 7255 represents the cDNA sequence for clone AA825779.

SEQ ID NO: 7256 represents the cDNA sequence for clone AA825781.

SEQ ID NO: 7257 represents the cDNA sequence for clone AA825782.

SEQ ID NO: 7258 represents the cDNA sequence for clone AA825783.

SEQ ID NO: 7259 represents the cDNA sequence for clone AA825784.

SEQ ID NO: 7260 represents the cDNA sequence for clone AA825785.

SEQ ID NO: 7261 represents the cDNA sequence for clone AA825788.

SEQ ID NO: 7262 represents the cDNA sequence for clone AA825790.

SEQ ID NO: 7263 represents the cDNA sequence for clone AA825792.

SEQ ID NO: 7264 represents the cDNA sequence for clone AA825795.

SEQ ID NO: 7265 represents the cDNA sequence for clone AA825796.

SEQ ID NO: 7266 represents the cDNA sequence for clone AA825798.

SEQ ID NO: 7267 represents the cDNA sequence for clone AA825799.

SEQ ID NO: 7268 represents the cDNA sequence for clone AA825800.

SEQ ID NO: 7269 represents the cDNA sequence for clone AA825802.

SEQ ID NO: 7270 represents the cDNA sequence for clone AA825803.

SEQ ID NO: 7271 represents the cDNA sequence for clone AA825804.

SEQ ID NO: 7272 represents the cDNA sequence for clone AA825805.

SEQ ID NO: 7273 represents the cDNA sequence for clone AA825807.

SEQ ID NO: 7274 represents the cDNA sequence for clone AA825808.

SEQ ID NO: 7275 represents the cDNA sequence for clone AA825999.

SEQ ID NO: 7276 represents the cDNA sequence for clone AA826006.

SEQ ID NO: 7277 represents the cDNA sequence for clone AA826009.

SEQ ID NO: 7278 represents the cDNA sequence for clone AA826010.

SEQ ID NO: 7279 represents the cDNA sequence for clone AA826011.

SEQ ID NO: 7280 represents the cDNA sequence for clone AA826012.

SEQ ID NO: 7281 represents the cDNA sequence for clone AA826015.

SEQ ID NO: 7282 represents the cDNA sequence for clone AA826016.

SEQ ID NO: 7283 represents the cDNA sequence for clone AA826018.

SEQ ID NO: 7284 represents the cDNA sequence for clone AA826019.

SEQ ID NO: 7285 represents the cDNA sequence for clone AA826020.

SEQ ID NO: 7286 represents the cDNA sequence for clone AA826021.

SEQ ID NO: 7287 represents the cDNA sequence for clone AA826030.

SEQ ID NO: 7288 represents the cDNA sequence for clone AA826036.

SEQ ID NO: 7289 represents the cDNA sequence for clone AA826131.

SEQ ID NO: 7290 represents the cDNA sequence for clone AA826138.

SEQ ID NO: 7291 represents the cDNA sequence for clone AA826139.

SEQ ID NO: 7292 represents the cDNA sequence for clone AA826144.

SEQ ID NO: 7293 represents the cDNA sequence for clone AA826148.

SEQ ID NO: 7294 represents the cDNA sequence for clone AA826151.

SEQ ID NO: 7295 represents the cDNA sequence for clone AA826152.

SEQ ID NO: 7296 represents the cDNA sequence for clone AA826158.

SEQ ID NO: 7297 represents the cDNA sequence for clone AA826159.

SEQ ID NO: 7298 represents the cDNA sequence for clone AA826160.

SEQ ID NO: 7299 represents the cDNA sequence for clone AA826162.

SEQ ID NO: 7300 represents the cDNA sequence for clone AA826168.

SEQ ID NO: 7301 represents the cDNA sequence for clone AA826169.

SEQ ID NO: 7302 represents the cDNA sequence for clone AA826292.

SEQ ID NO: 7303 represents the cDNA sequence for clone AA826293.

SEQ ID NO: 7304 represents the cDNA sequence for clone AA826294.

SEQ ID NO: 7305 represents the cDNA sequence for clone AA826297.

SEQ ID NO: 7306 represents the cDNA sequence for clone AA826298.

SEQ ID NO: 7307 represents the cDNA sequence for clone AA826299.

SEQ ID NO: 7308 represents the cDNA sequence for clone AA826300.

SEQ ID NO: 7309 represents the cDNA sequence for clone AA826301.

SEQ ID NO: 7310 represents the cDNA sequence for clone AA826305.

SEQ ID NO: 7311 represents the cDNA sequence for clone AA826306.

SEQ ID NO: 7312 represents the cDNA sequence for clone AA826307.

SEQ ID NO: 7313 represents the cDNA sequence for clone AA826309.

SEQ ID NO: 7314 represents the cDNA sequence for clone AA826310.

SEQ ID NO: 7315 represents the cDNA sequence for clone AA826311.

SEQ ID NO: 7316 represents the cDNA sequence for clone AA826312.

SEQ ID NO: 7317 represents the cDNA sequence for clone AA826313.

SEQ ID NO: 7318 represents the cDNA sequence for clone AA826314.

SEQ ID NO: 7319 represents the cDNA sequence for clone AA826316.

SEQ ID NO: 7320 represents the cDNA sequence for clone AA826320.

SEQ ID NO: 7321 represents the cDNA sequence for clone AA827949.

SEQ ID NO: 7322 represents the cDNA sequence for clone AA827951.

SEQ ID NO: 7323 represents the cDNA sequence for clone AA827953.

SEQ ID NO: 7324 represents the cDNA sequence for clone AA827960.

SEQ ID NO: 7325 represents the cDNA sequence for clone AA827961.

SEQ ID NO: 7326 represents the cDNA sequence for clone AA827962.

SEQ ID NO: 7327 represents the cDNA sequence for clone AA827964.

SEQ ID NO: 7328 represents the cDNA sequence for clone AA827966.

SEQ ID NO: 7329 represents the cDNA sequence for clone AA827967.

SEQ ID NO: 7330 represents the cDNA sequence for clone AA827970.

SEQ ID NO: 7331 represents the cDNA sequence for clone AA827971.

SEQ ID NO: 7332 represents the cDNA sequence for clone AA827972.

SEQ ID NO: 7333 represents the cDNA sequence for clone AA827974.

SEQ ID NO: 7334 represents the cDNA sequence for clone AA827976.

SEQ ID NO: 7335 represents the cDNA sequence for clone AA827977.

SEQ ID NO: 7336 represents the cDNA sequence for clone AA827978.

SEQ ID NO: 7337 represents the cDNA sequence for clone AA827979.

SEQ ID NO: 7338 represents the cDNA sequence for clone AA827981.

SEQ ID NO: 7339 represents the cDNA sequence for clone AA828036.

SEQ ID NO: 7340 represents the cDNA sequence for clone AA828038.

SEQ ID NO: 7341 represents the cDNA sequence for clone AA828039.

SEQ ID NO: 7342 represents the cDNA sequence for clone AA828040.

SEQ ID NO: 7343 represents the cDNA sequence for clone AA828042.

SEQ ID NO: 7344 represents the cDNA sequence for clone AA828044.

SEQ ID NO: 7345 represents the cDNA sequence for clone AA828046.

SEQ ID NO: 7346 represents the cDNA sequence for clone AA828048.

SEQ ID NO: 7347 represents the cDNA sequence for clone AA828049.

SEQ ID NO: 7348 represents the cDNA sequence for clone AA828050.

SEQ ID NO: 7349 represents the cDNA sequence for clone AA828053.

SEQ ID NO: 7350 represents the cDNA sequence for clone AA828054.

SEQ ID NO: 7351 represents the cDNA sequence for clone AA828056.

SEQ ID NO: 7352 represents the cDNA sequence for clone AA828058.

SEQ ID NO: 7353 represents the cDNA sequence for clone AA828060.

SEQ ID NO: 7354 represents the cDNA sequence for clone AA828061.

SEQ ID NO: 7355 represents the cDNA sequence for clone AA828063.

SEQ ID NO: 7356 represents the cDNA sequence for clone AA828123.

SEQ ID NO: 7357 represents the cDNA sequence for clone AA828125.

SEQ ID NO: 7358 represents the cDNA sequence for clone AA828126.

SEQ ID NO: 7359 represents the cDNA sequence for clone AA828127.

SEQ ID NO: 7360 represents the cDNA sequence for clone AA828130.

SEQ ID NO: 7361 represents the cDNA sequence for clone AA828131.

SEQ ID NO: 7362 represents the cDNA sequence for clone AA828137.

SEQ ID NO: 7363 represents the cDNA sequence for clone AA828138.

SEQ ID NO: 7364 represents the cDNA sequence for clone AA828140.

SEQ ID NO: 7365 represents the cDNA sequence for clone AA828141.

SEQ ID NO: 7366 represents the cDNA sequence for clone AA828145.

SEQ ID NO: 7367 represents the cDNA sequence for clone AA828148.

SEQ ID NO: 7368 represents the cDNA sequence for clone AA828152.

SEQ ID NO: 7369 represents the cDNA sequence for clone AA828153.

SEQ ID NO: 7370 represents the cDNA sequence for clone AA828557.

SEQ ID NO: 7371 represents the cDNA sequence for clone AA828559.

SEQ ID NO: 7372 represents the cDNA sequence for clone AA828560.

SEQ ID NO: 7373 represents the cDNA sequence for clone AA828563.

SEQ ID NO: 7374 represents the cDNA sequence for clone AA828566.

SEQ ID NO: 7375 represents the cDNA sequence for clone AA828567.

SEQ ID NO: 7376 represents the cDNA sequence for clone AA828571.

SEQ ID NO: 7377 represents the cDNA sequence for clone AA828573.

SEQ ID NO: 7378 represents the cDNA sequence for clone AA828575.

SEQ ID NO: 7379 represents the cDNA sequence for clone AA828576.

SEQ ID NO: 7380 represents the cDNA sequence for clone AA828593.

SEQ ID NO: 7381 represents the cDNA sequence for clone AA828596.

SEQ ID NO: 7382 represents the cDNA sequence for clone AA828597.

SEQ ID NO: 7383 represents the cDNA sequence for clone AA828598.

SEQ ID NO: 7384 represents the cDNA sequence for clone AA828603.

SEQ ID NO: 7385 represents the cDNA sequence for clone AA828604.

SEQ ID NO: 7386 represents the cDNA sequence for clone AA828607.

SEQ ID NO: 7387 represents the cDNA sequence for clone AA828609.

SEQ ID NO: 7388 represents the cDNA sequence for clone AA828611.

SEQ ID NO: 7389 represents the cDNA sequence for clone AA828612.

SEQ ID NO: 7390 represents the cDNA sequence for clone AA828613.

SEQ ID NO: 7391 represents the cDNA sequence for clone AA828614.

SEQ ID NO: 7392 represents the cDNA sequence for clone AA828616.

SEQ ID NO: 7393 represents the cDNA sequence for clone AA828617.

SEQ ID NO: 7394 represents the cDNA sequence for clone AA828618.

SEQ ID NO: 7395 represents the cDNA sequence for clone AA828627.

SEQ ID NO: 7396 represents the cDNA sequence for clone AA828628.

SEQ ID NO: 7397 represents the cDNA sequence for clone AA828629.

SEQ ID NO: 7398 represents the cDNA sequence for clone AA828630.

SEQ ID NO: 7399 represents the cDNA sequence for clone AA828636.

SEQ ID NO: 7400 represents the cDNA sequence for clone AA828640.

SEQ ID NO: 7401 represents the cDNA sequence for clone AA828643.

SEQ ID NO: 7402 represents the cDNA sequence for clone AA828646.

SEQ ID NO: 7403 represents the cDNA sequence for clone AA828647.

SEQ ID NO: 7404 represents the cDNA sequence for clone AA828648.

SEQ ID NO: 7405 represents the cDNA sequence for clone AA828650.

SEQ ID NO: 7406 represents the cDNA sequence for clone AA828651.

SEQ ID NO: 7407 represents the cDNA sequence for clone AA828653.

SEQ ID NO: 7408 represents the cDNA sequence for clone AA828655.

SEQ ID NO: 7409 represents the cDNA sequence for clone AA828656.

SEQ ID NO: 7410 represents the cDNA sequence for clone AA828657.

SEQ ID NO: 7411 represents the cDNA sequence for clone AA828658.

SEQ ID NO: 7412 represents the cDNA sequence for clone AA828663.

SEQ ID NO: 7413 represents the cDNA sequence for clone AA828664.

SEQ ID NO: 7414 represents the cDNA sequence for clone AA828665.

SEQ ID NO: 7415 represents the cDNA sequence for clone AA828666.

SEQ ID NO: 7416 represents the cDNA sequence for clone AA828668.

SEQ ID NO: 7417 represents the cDNA sequence for clone AA828669.

SEQ ID NO: 7418 represents the cDNA sequence for clone AA828670.

SEQ ID NO: 7419 represents the cDNA sequence for clone AA828672.

SEQ ID NO: 7420 represents the cDNA sequence for clone AA828674.

SEQ ID NO: 7421 represents the cDNA sequence for clone AA828677.

SEQ ID NO: 7422 represents the cDNA sequence for clone AA828684.

SEQ ID NO: 7423 represents the cDNA sequence for clone AA828685.

SEQ ID NO: 7424 represents the cDNA sequence for clone AA828688.

SEQ ID NO: 7425 represents the cDNA sequence for clone AA828689.

SEQ ID NO: 7426 represents the cDNA sequence for clone AA828690.

SEQ ID NO: 7427 represents the cDNA sequence for clone AA828695.

SEQ ID NO: 7428 represents the cDNA sequence for clone AA828696.

SEQ ID NO: 7429 represents the cDNA sequence for clone AA828697.

SEQ ID NO: 7430 represents the cDNA sequence for clone AA828698.

SEQ ID NO: 7431 represents the cDNA sequence for clone AA828699.

SEQ ID NO: 7432 represents the cDNA sequence for clone AA828700.

SEQ ID NO: 7433 represents the cDNA sequence for clone AA828701.

SEQ ID NO: 7434 represents the cDNA sequence for clone AA828702.

SEQ ID NO: 7435 represents the cDNA sequence for clone AA828703.

SEQ ID NO: 7436 represents the cDNA sequence for clone AA828704.

SEQ ID NO: 7437 represents the cDNA sequence for clone AA828710.

SEQ ID NO: 7438 represents the cDNA sequence for clone AA828711.

SEQ ID NO: 7439 represents the cDNA sequence for clone AA828712.

SEQ ID NO: 7440 represents the cDNA sequence for clone AA828713.

SEQ ID NO: 7441 represents the cDNA sequence for clone AA828714.

SEQ ID NO: 7442 represents the cDNA sequence for clone AA828717.

SEQ ID NO: 7443 represents the cDNA sequence for clone AA828723.

SEQ ID NO: 7444 represents the cDNA sequence for clone AA828724.

SEQ ID NO: 7445 represents the cDNA sequence for clone AA828726.

SEQ ID NO: 7446 represents the cDNA sequence for clone AA828727.

SEQ ID NO: 7447 represents the cDNA sequence for clone AA828728.

SEQ ID NO: 7448 represents the cDNA sequence for clone AA828729.

SEQ ID NO: 7449 represents the cDNA sequence for clone AA828732.

SEQ ID NO: 7450 represents the cDNA sequence for clone AA828733.

SEQ ID NO: 7451 represents the cDNA sequence for clone AA828734.

SEQ ID NO: 7452 represents the cDNA sequence for clone AA828735.

SEQ ID NO: 7453 represents the cDNA sequence for clone AA828738.

SEQ ID NO: 7454 represents the cDNA sequence for clone AA828740.

SEQ ID NO: 7455 represents the cDNA sequence for clone AA828745.

SEQ ID NO: 7456 represents the cDNA sequence for clone AA828750.

SEQ ID NO: 7457 represents the cDNA sequence for clone AA828754.

SEQ ID NO: 7458 represents the cDNA sequence for clone AA828760.

SEQ ID NO: 7459 represents the cDNA sequence for clone AA828762.

SEQ ID NO: 7460 represents the cDNA sequence for clone AA828763.

SEQ ID NO: 7461 represents the cDNA sequence for clone AA828765.

SEQ ID NO: 7462 represents the cDNA sequence for clone AA828766.

SEQ ID NO: 7463 represents the cDNA sequence for clone AA828774.

SEQ ID NO: 7464 represents the cDNA sequence for clone AA828775.

SEQ ID NO: 7465 represents the cDNA sequence for clone AA828776.

SEQ ID NO: 7466 represents the cDNA sequence for clone AA828777.

SEQ ID NO: 7467 represents the cDNA sequence for clone AA828778.

SEQ ID NO: 7468 represents the cDNA sequence for clone AA828779.

SEQ ID NO: 7469 represents the cDNA sequence for clone AA828780.

SEQ ID NO: 7470 represents the cDNA sequence for clone AA828783.

SEQ ID NO: 7471 represents the cDNA sequence for clone AA828784.

SEQ ID NO: 7472 represents the cDNA sequence for clone AA828785.

SEQ ID NO: 7473 represents the cDNA sequence for clone AA828787.

SEQ ID NO: 7474 represents the cDNA sequence for clone AA828788.

SEQ ID NO: 7475 represents the cDNA sequence for clone AA828791.

SEQ ID NO: 7476 represents the cDNA sequence for clone AA828792.

SEQ ID NO: 7477 represents the cDNA sequence for clone AA828793.

SEQ ID NO: 7478 represents the cDNA sequence for clone AA828794.

SEQ ID NO: 7479 represents the cDNA sequence for clone AA828795.

SEQ ID NO: 7480 represents the cDNA sequence for clone AA828796.

SEQ ID NO: 7481 represents the cDNA sequence for clone AA828800.

SEQ ID NO: 7482 represents the cDNA sequence for clone AA828801.

SEQ ID NO: 7483 represents the cDNA sequence for clone AA828805.

SEQ ID NO: 7484 represents the cDNA sequence for clone AA828807.

SEQ ID NO: 7485 represents the cDNA sequence for clone AA828808.

SEQ ID NO: 7486 represents the cDNA sequence for clone AA828809.

SEQ ID NO: 7487 represents the cDNA sequence for clone AA828810.

SEQ ID NO: 7488 represents the cDNA sequence for clone AA828811.

SEQ ID NO: 7489 represents the cDNA sequence for clone AA828812.

SEQ ID NO: 7490 represents the cDNA sequence for clone AA828816.

SEQ ID NO: 7491 represents the cDNA sequence for clone AA828817.

SEQ ID NO: 7492 represents the cDNA sequence for clone AA828818.

SEQ ID NO: 7493 represents the cDNA sequence for clone AA828822.

SEQ ID NO: 7494 represents the cDNA sequence for clone AA828823.

SEQ ID NO: 7495 represents the cDNA sequence for clone AA828825.

SEQ ID NO: 7496 represents the cDNA sequence for clone AA828826.

SEQ ID NO: 7497 represents the cDNA sequence for clone AA828827.

SEQ ID NO: 7498 represents the cDNA sequence for clone AA828828.

SEQ ID NO: 7499 represents the cDNA sequence for clone AA828830.

SEQ ID NO: 7500 represents the cDNA sequence for clone AA828831.

SEQ ID NO: 7501 represents the cDNA sequence for clone AA828832.

SEQ ID NO: 7502 represents the cDNA sequence for clone AA828833.

SEQ ID NO: 7503 represents the cDNA sequence for clone AA828834.

SEQ ID NO: 7504 represents the cDNA sequence for clone AA828835.

SEQ ID NO: 7505 represents the cDNA sequence for clone AA828836.

SEQ ID NO: 7506 represents the cDNA sequence for clone AA828837.

SEQ ID NO: 7507 represents the cDNA sequence for clone AA828838.

SEQ ID NO: 7508 represents the cDNA sequence for clone AA828839.

SEQ ID NO: 7509 represents the cDNA sequence for clone AA828840.

SEQ ID NO: 7510 represents the cDNA sequence for clone AA828841.

SEQ ID NO: 7511 represents the cDNA sequence for clone AA828843.

SEQ ID NO: 7512 represents the cDNA sequence for clone AA828845.

SEQ ID NO: 7513 represents the cDNA sequence for clone AA828850.

SEQ ID NO: 7514 represents the cDNA sequence for clone AA828852.

SEQ ID NO: 7515 represents the cDNA sequence for clone AA828853.

SEQ ID NO: 7516 represents the cDNA sequence for clone AA828854.

SEQ ID NO: 7517 represents the cDNA sequence for clone AA828860.

SEQ ID NO: 7518 represents the cDNA sequence for clone AA828865.

SEQ ID NO: 7519 represents the cDNA sequence for clone AA828866.

SEQ ID NO: 7520 represents the cDNA sequence for clone AA828868.

SEQ ID NO: 7521 represents the cDNA sequence for clone AA828870.

SEQ ID NO: 7522 represents the cDNA sequence for clone AA828875.

SEQ ID NO: 7523 represents the cDNA sequence for clone AA828876.

SEQ ID NO: 7524 represents the cDNA sequence for clone AA828878.

SEQ ID NO: 7525 represents the cDNA sequence for clone AA828880.

SEQ ID NO: 7526 represents the cDNA sequence for clone AA828881.

SEQ ID NO: 7527 represents the cDNA sequence for clone AA828882.

SEQ ID NO: 7528 represents the cDNA sequence for clone AA828886.

SEQ ID NO: 7529 represents the cDNA sequence for clone AA828891.

SEQ ID NO: 7530 represents the cDNA sequence for clone AA828892.

SEQ ID NO: 7531 represents the cDNA sequence for clone AA828894.

SEQ ID NO: 7532 represents the cDNA sequence for clone AA828895.

SEQ ID NO: 7533 represents the cDNA sequence for clone AA828897.

SEQ ID NO: 7534 represents the cDNA sequence for clone AA828899.

SEQ ID NO: 7535 represents the cDNA sequence for clone AA828903.

SEQ ID NO: 7536 represents the cDNA sequence for clone AA828904.

SEQ ID NO: 7537 represents the cDNA sequence for clone AA828909.

SEQ ID NO: 7538 represents the cDNA sequence for clone AA828911.

SEQ ID NO: 7539 represents the cDNA sequence for clone AA828912.

SEQ ID NO: 7540 represents the cDNA sequence for clone AA828913.

SEQ ID NO: 7541 represents the cDNA sequence for clone AA828917.

SEQ ID NO: 7542 represents the cDNA sequence for clone AA828922.

SEQ ID NO: 7543 represents the cDNA sequence for clone AA828924.

SEQ ID NO: 7544 represents the cDNA sequence for clone AA828925.

SEQ ID NO: 7545 represents the cDNA sequence for clone AA828926.

SEQ ID NO: 7546 represents the cDNA sequence for clone AA828928.

SEQ ID NO: 7547 represents the cDNA sequence for clone AA828931.

SEQ ID NO: 7548 represents the cDNA sequence for clone AA828932.

SEQ ID NO: 7549 represents the cDNA sequence for clone AA828936.

SEQ ID NO: 7550 represents the cDNA sequence for clone AA828937.

SEQ ID NO: 7551 represents the cDNA sequence for clone AA828938.

SEQ ID NO: 7552 represents the cDNA sequence for clone AA828939.

SEQ ID NO: 7553 represents the cDNA sequence for clone AA828940.

SEQ ID NO: 7554 represents the cDNA sequence for clone AA828945.

SEQ ID NO: 7555 represents the cDNA sequence for clone AA828946.

SEQ ID NO: 7556 represents the cDNA sequence for clone AA828947.

SEQ ID NO: 7557 represents the cDNA sequence for clone AA828953.

SEQ ID NO: 7558 represents the cDNA sequence for clone AA828955.

SEQ ID NO: 7559 represents the cDNA sequence for clone AA828956.

SEQ ID NO: 7560 represents the cDNA sequence for clone AA828957.

SEQ ID NO: 7561 represents the cDNA sequence for clone AA828959.

SEQ ID NO: 7562 represents the cDNA sequence for clone AA828960.

SEQ ID NO: 7563 represents the cDNA sequence for clone AA828962.

SEQ ID NO: 7564 represents the cDNA sequence for clone AA828964.

SEQ ID NO: 7565 represents the cDNA sequence for clone AA828965.

SEQ ID NO: 7566 represents the cDNA sequence for clone AA828966.

SEQ ID NO: 7567 represents the cDNA sequence for clone AA828967.

SEQ ID NO: 7568 represents the cDNA sequence for clone AA828968.

SEQ ID NO: 7569 represents the cDNA sequence for clone AA828970.

SEQ ID NO: 7570 represents the cDNA sequence for clone AA828973.

SEQ ID NO: 7571 represents the cDNA sequence for clone AA828974.

SEQ ID NO: 7572 represents the cDNA sequence for clone AA828976.

SEQ ID NO: 7573 represents the cDNA sequence for clone AA828977.

SEQ ID NO: 7574 represents the cDNA sequence for clone AA828980.

SEQ ID NO: 7575 represents the cDNA sequence for clone AA828982.

SEQ ID NO: 7576 represents the cDNA sequence for clone AA828988.

SEQ ID NO: 7577 represents the cDNA sequence for clone AA828989.

SEQ ID NO: 7578 represents the cDNA sequence for clone AA828991.

SEQ ID NO: 7579 represents the cDNA sequence for clone AA828992.

SEQ ID NO: 7580 represents the cDNA sequence for clone AA828995.

SEQ ID NO: 7581 represents the cDNA sequence for clone AA828998.

SEQ ID NO: 7582 represents the cDNA sequence for clone AA828999.

SEQ ID NO: 7583 represents the cDNA sequence for clone AA829000.

SEQ ID NO: 7584 represents the cDNA sequence for clone AA829001.

SEQ ID NO: 7585 represents the cDNA sequence for clone AA829002.

SEQ ID NO: 7586 represents the cDNA sequence for clone AA829004.

SEQ ID NO: 7587 represents the cDNA sequence for clone AA829006.

SEQ ID NO: 7588 represents the cDNA sequence for clone AA829007.

SEQ ID NO: 7589 represents the cDNA sequence for clone AA829008.

SEQ ID NO: 7590 represents the cDNA sequence for clone AA829010.

SEQ ID NO: 7591 represents the cDNA sequence for clone AA829018.

SEQ ID NO: 7592 represents the cDNA sequence for clone AA829023.

SEQ ID NO: 7593 represents the cDNA sequence for clone AA829024.

SEQ ID NO: 7594 represents the cDNA sequence for clone AA829027.

SEQ ID NO: 7595 represents the cDNA sequence for clone AA829029.

SEQ ID NO: 7596 represents the cDNA sequence for clone AA829034.

SEQ ID NO: 7597 represents the cDNA sequence for clone AA829038.

SEQ ID NO: 7598 represents the cDNA sequence for clone AA829041.

SEQ ID NO: 7599 represents the cDNA sequence for clone AA829042.

SEQ ID NO: 7600 represents the cDNA sequence for clone AA829043.

SEQ ID NO: 7601 represents the cDNA sequence for clone AA829045.

SEQ ID NO: 7602 represents the cDNA sequence for clone AA829047.

SEQ ID NO: 7603 represents the cDNA sequence for clone AA829049.

SEQ ID NO: 7604 represents the cDNA sequence for clone AA829050.

SEQ ID NO: 7605 represents the cDNA sequence for clone AA829054.

SEQ ID NO: 7606 represents the cDNA sequence for clone AA829055.

SEQ ID NO: 7607 represents the cDNA sequence for clone AA829056.

SEQ ID NO: 7608 represents the cDNA sequence for clone AA829063.

SEQ ID NO: 7609 represents the cDNA sequence for clone AA829065.

SEQ ID NO: 7610 represents the cDNA sequence for clone AA829067.

SEQ ID NO: 7611 represents the cDNA sequence for clone AA829068.

SEQ ID NO: 7612 represents the cDNA sequence for clone AA829070.

SEQ ID NO: 7613 represents the cDNA sequence for clone AA829071.

SEQ ID NO: 7614 represents the cDNA sequence for clone AA829072.

SEQ ID NO: 7615 represents the cDNA sequence for clone AA829074.

SEQ ID NO: 7616 represents the cDNA sequence for clone AA829075.

SEQ ID NO: 7617 represents the cDNA sequence for clone AA829077.

SEQ ID NO: 7618 represents the cDNA sequence for clone AA829078.

SEQ ID NO: 7619 represents the cDNA sequence for clone AA829081.

SEQ ID NO: 7620 represents the cDNA sequence for clone AA829082.

SEQ ID NO: 7621 represents the cDNA sequence for clone AA829084.

SEQ ID NO: 7622 represents the cDNA sequence for clone AA829088.

SEQ ID NO: 7623 represents the cDNA sequence for clone AA829089.

SEQ ID NO: 7624 represents the cDNA sequence for clone AA829090.

SEQ ID NO: 7625 represents the cDNA sequence for clone AA829091.

SEQ ID NO: 7626 represents the cDNA sequence for clone AA829093.

SEQ ID NO: 7627 represents the cDNA sequence for clone AA829094.

SEQ ID NO: 7628 represents the cDNA sequence for clone AA829095.

SEQ ID NO: 7629 represents the cDNA sequence for clone AA829096.

SEQ ID NO: 7630 represents the cDNA sequence for clone AA829097.

SEQ ID NO: 7631 represents the cDNA sequence for clone AA829098.

SEQ ID NO: 7632 represents the cDNA sequence for clone AA829099.

SEQ ID NO: 7633 represents the cDNA sequence for clone AA829103.

SEQ ID NO: 7634 represents the cDNA sequence for clone AA829106.

SEQ ID NO: 7635 represents the cDNA sequence for clone AA829111.

SEQ ID NO: 7636 represents the cDNA sequence for clone AA829112.

SEQ ID NO: 7637 represents the cDNA sequence for clone AA829114.

SEQ ID NO: 7638 represents the cDNA sequence for clone AA829115.

SEQ ID NO: 7639 represents the cDNA sequence for clone AA829116.

SEQ ID NO: 7640 represents the cDNA sequence for clone AA829117.

SEQ ID NO: 7641 represents the cDNA sequence for clone AA829120.

SEQ ID NO: 7642 represents the cDNA sequence for clone AA829127.

SEQ ID NO: 7643 represents the cDNA sequence for clone AA829131.

SEQ ID NO: 7644 represents the cDNA sequence for clone AA829132.

SEQ ID NO: 7645 represents the cDNA sequence for clone AA829133.

SEQ ID NO: 7646 represents the cDNA sequence for clone AA829134.

SEQ ID NO: 7647 represents the cDNA sequence for clone AA829135.

SEQ ID NO: 7648 represents the cDNA sequence for clone AA829136.

SEQ ID NO: 7649 represents the cDNA sequence for clone AA829137.

SEQ ID NO: 7650 represents the cDNA sequence for clone AA829138.

SEQ ID NO: 7651 represents the cDNA sequence for clone AA829140.

SEQ ID NO: 7652 represents the cDNA sequence for clone AA829142.

SEQ ID NO: 7653 represents the cDNA sequence for clone AA829145.

SEQ ID NO: 7654 represents the cDNA sequence for clone AA829153.

SEQ ID NO: 7655 represents the cDNA sequence for clone AA829157.

SEQ ID NO: 7656 represents the cDNA sequence for clone AA829159.

SEQ ID NO: 7657 represents the cDNA sequence for clone AA829161.

SEQ ID NO: 7658 represents the cDNA sequence for clone AA829165.

SEQ ID NO: 7659 represents the cDNA sequence for clone AA829166.

SEQ ID NO: 7660 represents the cDNA sequence for clone AA829167.

SEQ ID NO: 7661 represents the cDNA sequence for clone AA829170.

SEQ ID NO: 7662 represents the cDNA sequence for clone AA829171.

SEQ ID NO: 7663 represents the cDNA sequence for clone AA829174.

SEQ ID NO: 7664 represents the cDNA sequence for clone AA829178.

SEQ ID NO: 7665 represents the cDNA sequence for clone AA829182.

SEQ ID NO: 7666 represents the cDNA sequence for clone AA829183.

SEQ ID NO: 7667 represents the cDNA sequence for clone AA829185.

SEQ ID NO: 7668 represents the cDNA sequence for clone AA829186.

SEQ ID NO: 7669 represents the cDNA sequence for clone AA829190.

SEQ ID NO: 7670 represents the cDNA sequence for clone AA829192.

SEQ ID NO: 7671 represents the cDNA sequence for clone AA829194.

SEQ ID NO: 7672 represents the cDNA sequence for clone AA829196.

SEQ ID NO: 7673 represents the cDNA sequence for clone AA829197.

SEQ ID NO: 7674 represents the cDNA sequence for clone AA829200.

SEQ ID NO: 7675 represents the cDNA sequence for clone AA829202.

SEQ ID NO: 7676 represents the cDNA sequence for clone AA829203.

SEQ ID NO: 7677 represents the cDNA sequence for clone AA829206.

SEQ ID NO: 7678 represents the cDNA sequence for clone AA829209.

SEQ ID NO: 7679 represents the cDNA sequence for clone AA829211.

SEQ ID NO: 7680 represents the cDNA sequence for clone AA829213.

SEQ ID NO: 7681 represents the cDNA sequence for clone AA829219.

SEQ ID NO: 7682 represents the cDNA sequence for clone AA829222.

SEQ ID NO: 7683 represents the cDNA sequence for clone AA829224.

SEQ ID NO: 7684 represents the cDNA sequence for clone AA829557.

SEQ ID NO: 7685 represents the cDNA sequence for clone AA829559.

SEQ ID NO: 7686 represents the cDNA sequence for clone AA829560.

SEQ ID NO: 7687 represents the cDNA sequence for clone AA829561.

SEQ ID NO: 7688 represents the cDNA sequence for clone AA829563.

SEQ ID NO: 7689 represents the cDNA sequence for clone AA829567.

SEQ ID NO: 7690 represents the cDNA sequence for clone AA829568.

SEQ ID NO: 7691 represents the cDNA sequence for clone AA829572.

SEQ ID NO: 7692 represents the cDNA sequence for clone AA829577.

SEQ ID NO: 7693 represents the cDNA sequence for clone AA829579.

SEQ ID NO: 7694 represents the cDNA sequence for clone AA829582.

SEQ ID NO: 7695 represents the cDNA sequence for clone AA829584.

SEQ ID NO: 7696 represents the cDNA sequence for clone AA829590.

SEQ ID NO: 7697 represents the cDNA sequence for clone AA829593.

SEQ ID NO: 7698 represents the cDNA sequence for clone AA829594.

SEQ ID NO: 7699 represents the cDNA sequence for clone AA829664.

SEQ ID NO: 7700 represents the cDNA sequence for clone AA829666.

SEQ ID NO: 7701 represents the cDNA sequence for clone AA829668.

SEQ ID NO: 7702 represents the cDNA sequence for clone AA829671.

SEQ ID NO: 7703 represents the cDNA sequence for clone AA829672.

SEQ ID NO: 7704 represents the cDNA sequence for clone AA829675.

SEQ ID NO: 7705 represents the cDNA sequence for clone AA829684.

SEQ ID NO: 7706 represents the cDNA sequence for clone AA829685.

SEQ ID NO: 7707 represents the cDNA sequence for clone AA829690.

SEQ ID NO: 7708 represents the cDNA sequence for clone AA829691.

SEQ ID NO: 7709 represents the cDNA sequence for clone AA829692.

SEQ ID NO: 7710 represents the cDNA sequence for clone AA829694.

SEQ ID NO: 7711 represents the cDNA sequence for clone AA829696.

SEQ ID NO: 7712 represents the cDNA sequence for clone AA829697.

SEQ ID NO: 7713 represents the cDNA sequence for clone AA834620.

SEQ ID NO: 7714 represents the cDNA sequence for clone AA834622.

SEQ ID NO: 7715 represents the cDNA sequence for clone AA834624.

SEQ ID NO: 7716 represents the cDNA sequence for clone AA834625.

SEQ ID NO: 7717 represents the cDNA sequence for clone AA834626.

SEQ ID NO: 7718 represents the cDNA sequence for clone AA834629.

SEQ ID NO: 7719 represents the cDNA sequence for clone AA834633.

SEQ ID NO: 7720 represents the cDNA sequence for clone AA834639.

SEQ ID NO: 7721 represents the cDNA sequence for clone AA834647.

SEQ ID NO: 7722 represents the cDNA sequence for clone AA834652.

SEQ ID NO: 7723 represents the cDNA sequence for clone AA834653.

SEQ ID NO: 7724 represents the cDNA sequence for clone AA834656.

SEQ ID NO: 7725 represents the cDNA sequence for clone AA834659.

SEQ ID NO: 7726 represents the cDNA sequence for clone AA834660.

SEQ ID NO: 7727 represents the cDNA sequence for clone AA834661.

SEQ ID NO: 7728 represents the cDNA sequence for clone AA834662.

SEQ ID NO: 7729 represents the cDNA sequence for clone AA834663.

SEQ ID NO: 7730 represents the cDNA sequence for clone AA834665.

SEQ ID NO: 7731 represents the cDNA sequence for clone AA834667.

SEQ ID NO: 7732 represents the cDNA sequence for clone AA834669.

SEQ ID NO: 7733 represents the cDNA sequence for clone AA834671.

SEQ ID NO: 7734 represents the cDNA sequence for clone AA834672.

SEQ ID NO: 7735 represents the cDNA sequence for clone AA834678.

SEQ ID NO: 7736 represents the cDNA sequence for clone AA834679.

SEQ ID NO: 7737 represents the cDNA sequence for clone AA834681.

SEQ ID NO: 7738 represents the cDNA sequence for clone AA834683.

SEQ ID NO: 7739 represents the cDNA sequence for clone AA834684.

SEQ ID NO: 7740 represents the cDNA sequence for clone AA834686.

SEQ ID NO: 7741 represents the cDNA sequence for clone AA834687.

SEQ ID NO: 7742 represents the cDNA sequence for clone AA834692.

SEQ ID NO: 7743 represents the cDNA sequence for clone AA834697.

SEQ ID NO: 7744 represents the cDNA sequence for clone AA834699.

SEQ ID NO: 7745 represents the cDNA sequence for clone AA834702.

SEQ ID NO: 7746 represents the cDNA sequence for clone AA834704.

SEQ ID NO: 7747 represents the cDNA sequence for clone AA834705.

SEQ ID NO: 7748 represents the cDNA sequence for clone AA834709.

SEQ ID NO: 7749 represents the cDNA sequence for clone AA834710.

SEQ ID NO: 7750 represents the cDNA sequence for clone AA834711.

SEQ ID NO: 7751 represents the cDNA sequence for clone AA834712.

SEQ ID NO: 7752 represents the cDNA sequence for clone AA834717.

SEQ ID NO: 7753 represents the cDNA sequence for clone AA834718.

SEQ ID NO: 7754 represents the cDNA sequence for clone AA834721.

SEQ ID NO: 7755 represents the cDNA sequence for clone AA834723.

SEQ ID NO: 7756 represents the cDNA sequence for clone AA834724.

SEQ ID NO: 7757 represents the cDNA sequence for clone AA834728.

SEQ ID NO: 7758 represents the cDNA sequence for clone AA834729.

SEQ ID NO: 7759 represents the cDNA sequence for clone AA834730.

SEQ ID NO: 7760 represents the cDNA sequence for clone AA834731.

SEQ ID NO: 7761 represents the cDNA sequence for clone AA834732.

SEQ ID NO: 7762 represents the cDNA sequence for clone AA834734.

SEQ ID NO: 7763 represents the cDNA sequence for clone AA834739.

SEQ ID NO: 7764 represents the cDNA sequence for clone AA834740.

SEQ ID NO: 7765 represents the cDNA sequence for clone AA834741.

SEQ ID NO: 7766 represents the cDNA sequence for clone AA834742.

SEQ ID NO: 7767 represents the cDNA sequence for clone AA834743.

SEQ ID NO: 7768 represents the cDNA sequence for clone AA834744.

SEQ ID NO: 7769 represents the cDNA sequence for clone AA834746.

SEQ ID NO: 7770 represents the cDNA sequence for clone AA834747.

SEQ ID NO: 7771 represents the cDNA sequence for clone AA834748.

SEQ ID NO: 7772 represents the cDNA sequence for clone AA834750.

SEQ ID NO: 7773 represents the cDNA sequence for clone AA834752.

SEQ ID NO: 7774 represents the cDNA sequence for clone AA834757.

SEQ ID NO: 7775 represents the cDNA sequence for clone AA834758.

SEQ ID NO: 7776 represents the cDNA sequence for clone AA834759.

SEQ ID NO: 7777 represents the cDNA sequence for clone AA834761.

SEQ ID NO: 7778 represents the cDNA sequence for clone AA834762.

SEQ ID NO: 7779 represents the cDNA sequence for clone AA834763.

SEQ ID NO: 7780 represents the cDNA sequence for clone AA834764.

SEQ ID NO: 7781 represents the cDNA sequence for clone AA834765.

SEQ ID NO: 7782 represents the cDNA sequence for clone AA834769.

SEQ ID NO: 7783 represents the cDNA sequence for clone AA834770.

SEQ ID NO: 7784 represents the cDNA sequence for clone AA834775.

SEQ ID NO: 7785 represents the cDNA sequence for clone AA834778.

SEQ ID NO: 7786 represents the cDNA sequence for clone AA834780.

SEQ ID NO: 7787 represents the cDNA sequence for clone AA834781.

SEQ ID NO: 7788 represents the cDNA sequence for clone AA834782.

SEQ ID NO: 7789 represents the cDNA sequence for clone AA834785.

SEQ ID NO: 7790 represents the cDNA sequence for clone AA834786.

SEQ ID NO: 7791 represents the cDNA sequence for clone AA834787.

SEQ ID NO: 7792 represents the cDNA sequence for clone AA834789.

SEQ ID NO: 7793 represents the cDNA sequence for clone AA834793.

SEQ ID NO: 7794 represents the cDNA sequence for clone AA834795.

SEQ ID NO: 7795 represents the cDNA sequence for clone AA834796.

SEQ ID NO: 7796 represents the cDNA sequence for clone AA834797.

SEQ ID NO: 7797 represents the cDNA sequence for clone AA834800.

SEQ ID NO: 7798 represents the cDNA sequence for clone AA834802.

SEQ ID NO: 7799 represents the cDNA sequence for clone AA834803.

SEQ ID NO: 7800 represents the cDNA sequence for clone AA834805.

SEQ ID NO: 7801 represents the cDNA sequence for clone AA834806.

SEQ ID NO: 7802 represents the cDNA sequence for clone AA834809.

SEQ ID NO: 7803 represents the cDNA sequence for clone AA834810.

SEQ ID NO: 7804 represents the cDNA sequence for clone AA834812.

SEQ ID NO: 7805 represents the cDNA sequence for clone AA834813.

SEQ ID NO: 7806 represents the cDNA sequence for clone AA834814.

SEQ ID NO: 7807 represents the cDNA sequence for clone AA834815.

SEQ ID NO: 7808 represents the cDNA sequence for clone AA834817.

SEQ ID NO: 7809 represents the cDNA sequence for clone AA834820.

SEQ ID NO: 7810 represents the cDNA sequence for clone AA834823.

SEQ ID NO: 7811 represents the cDNA sequence for clone AA834825.

SEQ ID NO: 7812 represents the cDNA sequence for clone AA834829.

SEQ ID NO: 7813 represents the cDNA sequence for clone AA834832.

SEQ ID NO: 7814 represents the cDNA sequence for clone AA834833.

SEQ ID NO: 7815 represents the cDNA sequence for clone AA834834.

SEQ ID NO: 7816 represents the cDNA sequence for clone AA834838.

SEQ ID NO: 7817 represents the cDNA sequence for clone AA834841.

SEQ ID NO: 7818 represents the cDNA sequence for clone AA834842.

SEQ ID NO: 7819 represents the cDNA sequence for clone AA834844.

SEQ ID NO: 7820 represents the cDNA sequence for clone AA834851.

SEQ ID NO: 7821 represents the cDNA sequence for clone AA834854.

SEQ ID NO: 7822 represents the cDNA sequence for clone AA834856.

SEQ ID NO: 7823 represents the cDNA sequence for clone AA834857.

SEQ ID NO: 7824 represents the cDNA sequence for clone AA834858.

SEQ ID NO: 7825 represents the cDNA sequence for clone AA834859.

SEQ ID NO: 7826 represents the cDNA sequence for clone AA834860.

SEQ ID NO: 7827 represents the cDNA sequence for clone AA834861.

SEQ ID NO: 7828 represents the cDNA sequence for clone AA834862.

SEQ ID NO: 7829 represents the cDNA sequence for clone AA834869.

SEQ ID NO: 7830 represents the cDNA sequence for clone AA834870.

SEQ ID NO: 7831 represents the cDNA sequence for clone AA834874.

SEQ ID NO: 7832 represents the cDNA sequence for clone AA834876.

SEQ ID NO: 7833 represents the cDNA sequence for clone AA834878.

SEQ ID NO: 7834 represents the cDNA sequence for clone AA834879.

SEQ ID NO: 7835 represents the cDNA sequence for clone AA834880.

SEQ ID NO: 7836 represents the cDNA sequence for clone AA834883.

SEQ ID NO: 7837 represents the cDNA sequence for clone AA834887.

SEQ ID NO: 7838 represents the cDNA sequence for clone AA834889.

SEQ ID NO: 7839 represents the cDNA sequence for clone AA834892.

SEQ ID NO: 7840 represents the cDNA sequence for clone AA834893.

SEQ ID NO: 7841 represents the cDNA sequence for clone AA834895.

SEQ ID NO: 7842 represents the cDNA sequence for clone AA834896.

SEQ ID NO: 7843 represents the cDNA sequence for clone AA834898.

SEQ ID NO: 7844 represents the cDNA sequence for clone AA834901.

SEQ ID NO: 7845 represents the cDNA sequence for clone AA837215.

SEQ ID NO: 7846 represents the cDNA sequence for clone AA837221.

SEQ ID NO: 7847 represents the cDNA sequence for clone AA837224.

SEQ ID NO: 7848 represents the cDNA sequence for clone AA837225.

SEQ ID NO: 7849 represents the cDNA sequence for clone AA837226.

SEQ ID NO: 7850 represents the cDNA sequence for clone AA837227.

SEQ ID NO: 7851 represents the cDNA sequence for clone AA837228.

SEQ ID NO: 7852 represents the cDNA sequence for clone AA837229.

SEQ ID NO: 7853 represents the cDNA sequence for clone AA837232.

SEQ ID NO: 7854 represents the cDNA sequence for clone AA837233.

SEQ ID NO: 7855 represents the cDNA sequence for clone AA837234.

SEQ ID NO: 7856 represents the cDNA sequence for clone AA837238.

SEQ ID NO: 7857 represents the cDNA sequence for clone AA837239.

SEQ ID NO: 7858 represents the cDNA sequence for clone AA837242.

SEQ ID NO: 7859 represents the cDNA sequence for clone AA837243.

SEQ ID NO: 7860 represents the cDNA sequence for clone AA837245.

SEQ ID NO: 7861 represents the cDNA sequence for clone AA837328.

SEQ ID NO: 7862 represents the cDNA sequence for clone AA837331.

SEQ ID NO: 7863 represents the cDNA sequence for clone AA837332.

SEQ ID NO: 7864 represents the cDNA sequence for clone AA837336.

SEQ ID NO: 7865 represents the cDNA sequence for clone AA837339.

SEQ ID NO: 7866 represents the cDNA sequence for clone AA837341.

SEQ ID NO: 7867 represents the cDNA sequence for clone AA837346.

SEQ ID NO: 7868 represents the cDNA sequence for clone AA837349.

SEQ ID NO: 7869 represents the cDNA sequence for clone AA837354.

SEQ ID NO: 7870 represents the cDNA sequence for clone AA837357.

SEQ ID NO: 7871 represents the cDNA sequence for clone AA837358.

SEQ ID NO: 7872 represents the cDNA sequence for clone AA837583.

SEQ ID NO: 7873 represents the cDNA sequence for clone AA837584.

SEQ ID NO: 7874 represents the cDNA sequence for clone AA837588.

SEQ ID NO: 7875 represents the cDNA sequence for clone AA837592.

SEQ ID NO: 7876 represents the cDNA sequence for clone AA837594.

SEQ ID NO: 7877 represents the cDNA sequence for clone AA837596.

SEQ ID NO: 7878 represents the cDNA sequence for clone AA837597.

SEQ ID NO: 7879 represents the cDNA sequence for clone AA837599.

SEQ ID NO: 7880 represents the cDNA sequence for clone AA837600.

SEQ ID NO: 7881 represents the cDNA sequence for clone AA837606.

SEQ ID NO: 7882 represents the cDNA sequence for clone AA837608.

SEQ ID NO: 7883 represents the cDNA sequence for clone AA837610.

SEQ ID NO: 7884 represents the cDNA sequence for clone AA837612.

SEQ ID NO: 7885 represents the cDNA sequence for clone AA837613.

SEQ ID NO: 7886 represents the cDNA sequence for clone AA837615.

SEQ ID NO: 7887 represents the cDNA sequence for clone AA837616.

SEQ ID NO: 7888 represents the cDNA sequence for clone AA837618.

SEQ ID NO: 7889 represents the cDNA sequence for clone AA837621.

SEQ ID NO: 7890 represents the cDNA sequence for clone AA837622.

SEQ ID NO: 7891 represents the cDNA sequence for clone AA837623.

SEQ ID NO: 7892 represents the cDNA sequence for clone AA837624.

SEQ ID NO: 7893 represents the cDNA sequence for clone AA837625.

SEQ ID NO: 7894 represents the cDNA sequence for clone AA837630.

SEQ ID NO: 7895 represents the cDNA sequence for clone AA837634.

SEQ ID NO: 7896 represents the cDNA sequence for clone AA837635.

SEQ ID NO: 7897 represents the cDNA sequence for clone AA837638.

SEQ ID NO: 7898 represents the cDNA sequence for clone AA837640.

SEQ ID NO: 7899 represents the cDNA sequence for clone AA837641.

SEQ ID NO: 7900 represents the cDNA sequence for clone AA837644.

SEQ ID NO: 7901 represents the cDNA sequence for clone AA837646.

SEQ ID NO: 7902 represents the cDNA sequence for clone AA837647.

SEQ ID NO: 7903 represents the cDNA sequence for clone AA837649.

SEQ ID NO: 7904 represents the cDNA sequence for clone AA837652.

SEQ ID NO: 7905 represents the cDNA sequence for clone AA837653.

SEQ ID NO: 7906 represents the cDNA sequence for clone AA837654.

SEQ ID NO: 7907 represents the cDNA sequence for clone AA837656.

SEQ ID NO: 7908 represents the cDNA sequence for clone AA837659.

SEQ ID NO: 7909 represents the cDNA sequence for clone AA837661.

SEQ ID NO: 7910 represents the cDNA sequence for clone AA837662.

SEQ ID NO: 7911 represents the cDNA sequence for clone AA837663.

SEQ ID NO: 7912 represents the cDNA sequence for clone AA837664.

SEQ ID NO: 7913 represents the cDNA sequence for clone AA837665.

SEQ ID NO: 7914 represents the cDNA sequence for clone AA837666.

SEQ ID NO: 7915 represents the cDNA sequence for clone AA837668.

SEQ ID NO: 7916 represents the cDNA sequence for clone AA837669.

SEQ ID NO: 7917 represents the cDNA sequence for clone AA837670.

SEQ ID NO: 7918 represents the cDNA sequence for clone AA837674.

SEQ ID NO: 7919 represents the cDNA sequence for clone AA837676.

SEQ ID NO: 7920 represents the cDNA sequence for clone AA837679.

SEQ ID NO: 7921 represents the cDNA sequence for clone AA837680.

SEQ ID NO: 7922 represents the cDNA sequence for clone AA837681.

SEQ ID NO: 7923 represents the cDNA sequence for clone AA837683.

SEQ ID NO: 7924 represents the cDNA sequence for clone AA837685.

SEQ ID NO: 7925 represents the cDNA sequence for clone AA837686.

SEQ ID NO: 7926 represents the cDNA sequence for clone AA837687.

SEQ ID NO: 7927 represents the cDNA sequence for clone AA837689.

SEQ ID NO: 7928 represents the cDNA sequence for clone AA837694.

SEQ ID NO: 7929 represents the cDNA sequence for clone AA837700.

SEQ ID NO: 7930 represents the cDNA sequence for clone AA837703.

SEQ ID NO: 7931 represents the cDNA sequence for clone AA837704.

SEQ ID NO: 7932 represents the cDNA sequence for clone AA837705.

SEQ ID NO: 7933 represents the cDNA sequence for clone AA837706.

SEQ ID NO: 7934 represents the cDNA sequence for clone AA837709.

SEQ ID NO: 7935 represents the cDNA sequence for clone AA837710.

SEQ ID NO: 7936 represents the cDNA sequence for clone AA837711.

SEQ ID NO: 7937 represents the cDNA sequence for clone AA837714.

SEQ ID NO: 7938 represents the cDNA sequence for clone AA837716.

SEQ ID NO: 7939 represents the cDNA sequence for clone AA837720.

SEQ ID NO: 7940 represents the cDNA sequence for clone AA837721.

SEQ ID NO: 7941 represents the cDNA sequence for clone AA837722.

SEQ ID NO: 7942 represents the cDNA sequence for clone AA837724.

SEQ ID NO: 7943 represents the cDNA sequence for clone AA837725.

SEQ ID NO: 7944 represents the cDNA sequence for clone AA837726.

SEQ ID NO: 7945 represents the cDNA sequence for clone AA837731.

SEQ ID NO: 7946 represents the cDNA sequence for clone AA837734.

SEQ ID NO: 7947 represents the cDNA sequence for clone AA837735.

SEQ ID NO: 7948 represents the cDNA sequence for clone AA837737.

SEQ ID NO: 7949 represents the cDNA sequence for clone AA837738.

SEQ ID NO: 7950 represents the cDNA sequence for clone AA837741.

SEQ ID NO: 7951 represents the cDNA sequence for clone AA837742.

SEQ ID NO: 7952 represents the cDNA sequence for clone AA837743.

SEQ ID NO: 7953 represents the cDNA sequence for clone AA837744.

SEQ ID NO: 7954 represents the cDNA sequence for clone AA837747.

SEQ ID NO: 7955 represents the cDNA sequence for clone AA837748.

SEQ ID NO: 7956 represents the cDNA sequence for clone AA837750.

SEQ ID NO: 7957 represents the cDNA sequence for clone AA837754.

SEQ ID NO: 7958 represents the cDNA sequence for clone AA837755.

SEQ ID NO: 7959 represents the cDNA sequence for clone AA837759.

SEQ ID NO: 7960 represents the cDNA sequence for clone AA837760.

SEQ ID NO: 7961 represents the cDNA sequence for clone AA837762.

SEQ ID NO: 7962 represents the cDNA sequence for clone AA837765.

SEQ ID NO: 7963 represents the cDNA sequence for clone AA837766.

SEQ ID NO: 7964 represents the cDNA sequence for clone AA837770.

SEQ ID NO: 7965 represents the cDNA sequence for clone AA837771.

SEQ ID NO: 7966 represents the cDNA sequence for clone AA837772.

SEQ ID NO: 7967 represents the cDNA sequence for clone AA837774.

SEQ ID NO: 7968 represents the cDNA sequence for clone AA837775.

SEQ ID NO: 7969 represents the cDNA sequence for clone AA837777.

SEQ ID NO: 7970 represents the cDNA sequence for clone AA837779.

SEQ ID NO: 7971 represents the cDNA sequence for clone AA837782.

SEQ ID NO: 7972 represents the cDNA sequence for clone AA837783.

SEQ ID NO: 7973 represents the cDNA sequence for clone AA837784.

SEQ ID NO: 7974 represents the cDNA sequence for clone AA837785.

SEQ ID NO: 7975 represents the cDNA sequence for clone AA837790.

SEQ ID NO: 7976 represents the cDNA sequence for clone AA837794.

SEQ ID NO: 7977 represents the cDNA sequence for clone AA837795.

SEQ ID NO: 7978 represents the cDNA sequence for clone AA837797.

SEQ ID NO: 7979 represents the cDNA sequence for clone AA837798.

SEQ ID NO: 7980 represents the cDNA sequence for clone AA837799.

SEQ ID NO: 7981 represents the cDNA sequence for clone AA837800.

SEQ ID NO: 7982 represents the cDNA sequence for clone AA837802.

SEQ ID NO: 7983 represents the cDNA sequence for clone AA838083.

SEQ ID NO: 7984 represents the cDNA sequence for clone AA838090.

SEQ ID NO: 7985 represents the cDNA sequence for clone AA838092.

SEQ ID NO: 7986 represents the cDNA sequence for clone AA838093.

SEQ ID NO: 7987 represents the cDNA sequence for clone AA838095.

SEQ ID NO: 7988 represents the cDNA sequence for clone AA838096.

SEQ ID NO: 7989 represents the cDNA sequence for clone AA838097.

SEQ ID NO: 7990 represents the cDNA sequence for clone AA838098.

SEQ ID NO: 7991 represents the cDNA sequence for clone AA838099.

SEQ ID NO: 7992 represents the cDNA sequence for clone AA838101.

SEQ ID NO: 7993 represents the cDNA sequence for clone AA838102.

SEQ ID NO: 7994 represents the cDNA sequence for clone AA838103.

SEQ ID NO: 7995 represents the cDNA sequence for clone AA838104.

SEQ ID NO: 7996 represents the cDNA sequence for clone AA838106.

SEQ ID NO: 7997 represents the cDNA sequence for clone AA838107.

SEQ ID NO: 7998 represents the cDNA sequence for clone AA838108.

SEQ ID NO: 7999 represents the cDNA sequence for clone AA838110.

SEQ ID NO: 8000 represents the cDNA sequence for clone AA838113.

SEQ ID NO: 8001 represents the cDNA sequence for clone AA838114.

SEQ ID NO: 8002 represents the cDNA sequence for clone AA838116.

SEQ ID NO: 8003 represents the cDNA sequence for clone AA838119.

SEQ ID NO: 8004 represents the cDNA sequence for clone AA838120.

SEQ ID NO: 8005 represents the cDNA sequence for clone AA838122.

SEQ ID NO: 8006 represents the cDNA sequence for clone AA838124.

SEQ ID NO: 8007 represents the cDNA sequence for clone AA838125.

SEQ ID NO: 8008 represents the cDNA sequence for clone AA838128.

SEQ ID NO: 8009 represents the cDNA sequence for clone AA838136.

SEQ ID NO: 8010 represents the cDNA sequence for clone AA838138.

SEQ ID NO: 8011 represents the cDNA sequence for clone AA838139.

SEQ ID NO: 8012 represents the cDNA sequence for clone AA838141.

SEQ ID NO: 8013 represents the cDNA sequence for clone AA838143.

SEQ ID NO: 8014 represents the cDNA sequence for clone AA838145.

SEQ ID NO: 8015 represents the cDNA sequence for clone AA838146.

SEQ ID NO: 8016 represents the cDNA sequence for clone AA838149.

SEQ ID NO: 8017 represents the cDNA sequence for clone AA838151.

SEQ ID NO: 8018 represents the cDNA sequence for clone AA838156.

SEQ ID NO: 8019 represents the cDNA sequence for clone AA838160.

SEQ ID NO: 8020 represents the cDNA sequence for clone AA838162.

SEQ ID NO: 8021 represents the cDNA sequence for clone AA838166.

SEQ ID NO: 8022 represents the cDNA sequence for clone AA838167.

SEQ ID NO: 8023 represents the cDNA sequence for clone AA838169.

SEQ ID NO: 8024 represents the cDNA sequence for clone AA838172.

SEQ ID NO: 8025 represents the cDNA sequence for clone AA838175.

SEQ ID NO: 8026 represents the cDNA sequence for clone AA838176.

SEQ ID NO: 8027 represents the cDNA sequence for clone AA838177.

SEQ ID NO: 8028 represents the cDNA sequence for clone AA838184.

SEQ ID NO: 8029 represents the cDNA sequence for clone AA838186.

SEQ ID NO: 8030 represents the cDNA sequence for clone AA838187.

SEQ ID NO: 8031 represents the cDNA sequence for clone AA838188.

SEQ ID NO: 8032 represents the cDNA sequence for clone AA838192.

SEQ ID NO: 8033 represents the cDNA sequence for clone AA838194.

SEQ ID NO: 8034 represents the cDNA sequence for clone AA838200.

SEQ ID NO: 8035 represents the cDNA sequence for clone AA838201.

SEQ ID NO: 8036 represents the cDNA sequence for clone AA838202.

SEQ ID NO: 8037 represents the cDNA sequence for clone AA838203.

SEQ ID NO: 8038 represents the cDNA sequence for clone AA838204.

SEQ ID NO: 8039 represents the cDNA sequence for clone AA838205.

SEQ ID NO: 8040 represents the cDNA sequence for clone AA838206.

SEQ ID NO: 8041 represents the cDNA sequence for clone AA838207.

SEQ ID NO: 8042 represents the cDNA sequence for clone AA838208.

SEQ ID NO: 8043 represents the cDNA sequence for clone AA846893.

SEQ ID NO: 8044 represents the cDNA sequence for clone AA846894.

SEQ ID NO: 8045 represents the cDNA sequence for clone AA846895.

SEQ ID NO: 8046 represents the cDNA sequence for clone AA846900.

SEQ ID NO: 8047 represents the cDNA sequence for clone AA846901.

SEQ ID NO: 8048 represents the cDNA sequence for clone AA846903.

SEQ ID NO: 8049 represents the cDNA sequence for clone AA846905.

SEQ ID NO: 8050 represents the cDNA sequence for clone AA846914.

SEQ ID NO: 8051 represents the cDNA sequence for clone AA846917.

SEQ ID NO: 8052 represents the cDNA sequence for clone AA846918.

SEQ ID NO: 8053 represents the cDNA sequence for clone AA846919.

SEQ ID NO: 8054 represents the cDNA sequence for clone AA846921.

SEQ ID NO: 8055 represents the cDNA sequence for clone AA846922.

SEQ ID NO: 8056 represents the cDNA sequence for clone AA846925.

SEQ ID NO: 8057 represents the cDNA sequence for clone AA846926.

SEQ ID NO: 8058 represents the cDNA sequence for clone AA846927.

SEQ ID NO: 8059 represents the cDNA sequence for clone AA846932.

SEQ ID NO: 8060 represents the cDNA sequence for clone AA846934.

SEQ ID NO: 8061 represents the cDNA sequence for clone AA846938.

SEQ ID NO: 8062 represents the cDNA sequence for clone AA846942.

SEQ ID NO: 8063 represents the cDNA sequence for clone AA846944.

SEQ ID NO: 8064 represents the cDNA sequence for clone AA846946.

SEQ ID NO: 8065 represents the cDNA sequence for clone AA846947.

SEQ ID NO: 8066 represents the cDNA sequence for clone AA846950.

SEQ ID NO: 8067 represents the cDNA sequence for clone AA846951.

SEQ ID NO: 8068 represents the cDNA sequence for clone AA846954.

SEQ ID NO: 8069 represents the cDNA sequence for clone AA846955.

SEQ ID NO: 8070 represents the cDNA sequence for clone AA846958.

SEQ ID NO: 8071 represents the cDNA sequence for clone AA846962.

SEQ ID NO: 8072 represents the cDNA sequence for clone AA846964.

SEQ ID NO: 8073 represents the cDNA sequence for clone AA846966.

SEQ ID NO: 8074 represents the cDNA sequence for clone AA846967.

SEQ ID NO: 8075 represents the cDNA sequence for clone AA846968.

SEQ ID NO: 8076 represents the cDNA sequence for clone AA846971.

SEQ ID NO: 8077 represents the cDNA sequence for clone AA846973.

SEQ ID NO: 8078 represents the cDNA sequence for clone AA846974.

SEQ ID NO: 8079 represents the cDNA sequence for clone AA846977.

SEQ ID NO: 8080 represents the cDNA sequence for clone AA846978.

SEQ ID NO: 8081 represents the cDNA sequence for clone AA846980.

SEQ ID NO: 8082 represents the cDNA sequence for clone AA846981.

SEQ ID NO: 8083 represents the cDNA sequence for clone AA846984.

SEQ ID NO: 8084 represents the cDNA sequence for clone AA846988.

SEQ ID NO: 8085 represents the cDNA sequence for clone AA846992.

SEQ ID NO: 8086 represents the cDNA sequence for clone AA846994.

SEQ ID NO: 8087 represents the cDNA sequence for clone AA846997.

SEQ ID NO: 8088 represents the cDNA sequence for clone AA847006.

SEQ ID NO: 8089 represents the cDNA sequence for clone AA847007.

SEQ ID NO: 8090 represents the cDNA sequence for clone AA847010.

SEQ ID NO: 8091 represents the cDNA sequence for clone AA847013.

SEQ ID NO: 8092 represents the cDNA sequence for clone AA847014.

SEQ ID NO: 8093 represents the cDNA sequence for clone AA847015.

SEQ ID NO: 8094 represents the cDNA sequence for clone AA847019.

SEQ ID NO: 8095 represents the cDNA sequence for clone AA847021.

SEQ ID NO: 8096 represents the cDNA sequence for clone AA847023.

SEQ ID NO: 8097 represents the cDNA sequence for clone AA847024.

SEQ ID NO: 8098 represents the cDNA sequence for clone AA847025.

SEQ ID NO: 8099 represents the cDNA sequence for clone AA847027.

SEQ ID NO: 8100 represents the cDNA sequence for clone AA847028.

SEQ ID NO: 8101 represents the cDNA sequence for clone AA847029.

SEQ ID NO: 8102 represents the cDNA sequence for clone AA847030.

SEQ ID NO: 8103 represents the cDNA sequence for clone AA847037.

SEQ ID NO: 8104 represents the cDNA sequence for clone AA847039.

SEQ ID NO: 8105 represents the cDNA sequence for clone AA847040.

SEQ ID NO: 8106 represents the cDNA sequence for clone AA847041.

SEQ ID NO: 8107 represents the cDNA sequence for clone AA847042.

SEQ ID NO: 8108 represents the cDNA sequence for clone AA847043.

SEQ ID NO: 8109 represents the cDNA sequence for clone AA847044.

SEQ ID NO: 8110 represents the cDNA sequence for clone AA847045.

SEQ ID NO: 8111 represents the cDNA sequence for clone AA847047.

SEQ ID NO: 8112 represents the cDNA sequence for clone AA847050.

SEQ ID NO: 8113 represents the cDNA sequence for clone AA847051.

SEQ ID NO: 8114 represents the cDNA sequence for clone AA847053.

SEQ ID NO: 8115 represents the cDNA sequence for clone AA847054.

SEQ ID NO: 8116 represents the cDNA sequence for clone AA847055.

SEQ ID NO: 8117 represents the cDNA sequence for clone AA847058.

SEQ ID NO: 8118 represents the cDNA sequence for clone AA847060.

SEQ ID NO: 8119 represents the cDNA sequence for clone AA847061.

SEQ ID NO: 8120 represents the cDNA sequence for clone AA847062.

SEQ ID NO: 8121 represents the cDNA sequence for clone AA847064.

SEQ ID NO: 8122 represents the cDNA sequence for clone AA847067.

SEQ ID NO: 8123 represents the cDNA sequence for clone AA847068.

SEQ ID NO: 8124 represents the cDNA sequence for clone AA847070.

SEQ ID NO: 8125 represents the cDNA sequence for clone AA847071.

SEQ ID NO: 8126 represents the cDNA sequence for clone AA847073.

SEQ ID NO: 8127 represents the cDNA sequence for clone AA847075.

SEQ ID NO: 8128 represents the cDNA sequence for clone AA847076.

SEQ ID NO: 8129 represents the cDNA sequence for clone AA847077.

SEQ ID NO: 8130 represents the cDNA sequence for clone AA847078.

SEQ ID NO: 8131 represents the cDNA sequence for clone AA847081.

SEQ ID NO: 8132 represents the cDNA sequence for clone AA847082.

SEQ ID NO: 8133 represents the cDNA sequence for clone AA847083.

SEQ ID NO: 8134 represents the cDNA sequence for clone AA847085.

SEQ ID NO: 8135 represents the cDNA sequence for clone AA847086.

SEQ ID NO: 8136 represents the cDNA sequence for clone AA847088.

SEQ ID NO: 8137 represents the cDNA sequence for clone AA847091.

SEQ ID NO: 8138 represents the cDNA sequence for clone AA847092.

SEQ ID NO: 8139 represents the cDNA sequence for clone AA847095.

SEQ ID NO: 8140 represents the cDNA sequence for clone AA847096.

SEQ ID NO: 8141 represents the cDNA sequence for clone AA847097.

SEQ ID NO: 8142 represents the cDNA sequence for clone AA847099.

SEQ ID NO: 8143 represents the cDNA sequence for clone AA847102.

SEQ ID NO: 8144 represents the cDNA sequence for clone AA847103.

SEQ ID NO: 8145 represents the cDNA sequence for clone AA847105.

SEQ ID NO: 8146 represents the cDNA sequence for clone AA847111.

SEQ ID NO: 8147 represents the cDNA sequence for clone AA847115.

SEQ ID NO: 8148 represents the cDNA sequence for clone AA847116.

SEQ ID NO: 8149 represents the cDNA sequence for clone AA847117.

SEQ ID NO: 8150 represents the cDNA sequence for clone AA847119.

SEQ ID NO: 8151 represents the cDNA sequence for clone AA847122.

SEQ ID NO: 8152 represents the cDNA sequence for clone AA847128.

SEQ ID NO: 8153 represents the cDNA sequence for clone AA847211.

SEQ ID NO: 8154 represents the cDNA sequence for clone AA847212.

SEQ ID NO: 8155 represents the cDNA sequence for clone AA847213.

SEQ ID NO: 8156 represents the cDNA sequence for clone AA847215.

SEQ ID NO: 8157 represents the cDNA sequence for clone AA847216.

SEQ ID NO: 8158 represents the cDNA sequence for clone AA847223.

SEQ ID NO: 8159 represents the cDNA sequence for clone AA847264.

SEQ ID NO: 8160 represents the cDNA sequence for clone AA847265.

SEQ ID NO: 8161 represents the cDNA sequence for clone AA847271.

SEQ ID NO: 8162 represents the cDNA sequence for clone AA847273.

SEQ ID NO: 8163 represents the cDNA sequence for clone AA847274.

SEQ ID NO: 8164 represents the cDNA sequence for clone AA847316.

SEQ ID NO: 8165 represents the cDNA sequence for clone AA847317.

SEQ ID NO: 8166 represents the cDNA sequence for clone AA847318.

SEQ ID NO: 8167 represents the cDNA sequence for clone AA847319.

SEQ ID NO: 8168 represents the cDNA sequence for clone AA847321.

SEQ ID NO: 8169 represents the cDNA sequence for clone AA847323.

SEQ ID NO: 8170 represents the cDNA sequence for clone AA847324.

SEQ ID NO: 8171 represents the cDNA sequence for clone AA847325.

SEQ ID NO: 8172 represents the cDNA sequence for clone AA847327.

SEQ ID NO: 8173 represents the cDNA sequence for clone AA847333.

SEQ ID NO: 8174 represents the cDNA sequence for clone AA847335.

SEQ ID NO: 8175 represents the cDNA sequence for clone AA847338.

SEQ ID NO: 8176 represents the cDNA sequence for clone AA847339.

SEQ ID NO: 8177 represents the cDNA sequence for clone AA847342.

SEQ ID NO: 8178 represents the cDNA sequence for clone AA847345.

SEQ ID NO: 8179 represents the cDNA sequence for clone AA847387.

SEQ ID NO: 8180 represents the cDNA sequence for clone AA847389.

SEQ ID NO: 8181 represents the cDNA sequence for clone AA847390.

SEQ ID NO: 8182 represents the cDNA sequence for clone AA847392.

SEQ ID NO: 8183 represents the cDNA sequence for clone AA847393.

SEQ ID NO: 8184 represents the cDNA sequence for clone AA847395.

SEQ ID NO: 8185 represents the cDNA sequence for clone AA847396.

SEQ ID NO: 8186 represents the cDNA sequence for clone AA847397.

SEQ ID NO: 8187 represents the cDNA sequence for clone AA847398.

SEQ ID NO: 8188 represents the cDNA sequence for clone AA847400.

SEQ ID NO: 8189 represents the cDNA sequence for clone AA847401.

SEQ ID NO: 8190 represents the cDNA sequence for clone AA847402.

SEQ ID NO: 8191 represents the cDNA sequence for clone AA847403.

SEQ ID NO: 8192 represents the cDNA sequence for clone AA847406.

SEQ ID NO: 8193 represents the cDNA sequence for clone AA847410.

SEQ ID NO: 8194 represents the cDNA sequence for clone AA847412.

SEQ ID NO: 8195 represents the cDNA sequence for clone AA847413.

SEQ ID NO: 8196 represents the cDNA sequence for clone AA847414.

SEQ ID NO: 8197 represents the cDNA sequence for clone AA847417.

SEQ ID NO: 8198 represents the cDNA sequence for clone AA847418.

SEQ ID NO: 8199 represents the cDNA sequence for clone AA847419.

SEQ ID NO: 8200 represents the cDNA sequence for clone AA847422.

SEQ ID NO: 8201 represents the cDNA sequence for clone AA847423.

SEQ ID NO: 8202 represents the cDNA sequence for clone AA847425.

SEQ ID NO: 8203 represents the cDNA sequence for clone AA847429.

SEQ ID NO: 8204 represents the cDNA sequence for clone AA847500.

SEQ ID NO: 8205 represents the cDNA sequence for clone AA847505.

SEQ ID NO: 8206 represents the cDNA sequence for clone AA847509.

SEQ ID NO: 8207 represents the cDNA sequence for clone AA847510.

SEQ ID NO: 8208 represents the cDNA sequence for clone AA847511.

SEQ ID NO: 8209 represents the cDNA sequence for clone AA847512.

SEQ ID NO: 8210 represents the cDNA sequence for clone AA847513.

SEQ ID NO: 8211 represents the cDNA sequence for clone AA847514.

SEQ ID NO: 8212 represents the cDNA sequence for clone AA847515.

SEQ ID NO: 8213 represents the cDNA sequence for clone AA847517.

SEQ ID NO: 8214 represents the cDNA sequence for clone AA847518.

SEQ ID NO: 8215 represents the cDNA sequence for clone AA847525.

SEQ ID NO: 8216 represents the cDNA sequence for clone AA847608.

SEQ ID NO: 8217 represents the cDNA sequence for clone AA847611.

SEQ ID NO: 8218 represents the cDNA sequence for clone AA847612.

SEQ ID NO: 8219 represents the cDNA sequence for clone AA847614.

SEQ ID NO: 8220 represents the cDNA sequence for clone AA847619.

SEQ ID NO: 8221 represents the cDNA sequence for clone AA847620.

SEQ ID NO: 8222 represents the cDNA sequence for clone AA847624.

SEQ ID NO: 8223 represents the cDNA sequence for clone AA847691.

SEQ ID NO: 8224 represents the cDNA sequence for clone AA847694.

SEQ ID NO: 8225 represents the cDNA sequence for clone AA847696.

SEQ ID NO: 8226 represents the cDNA sequence for clone AA847697.

SEQ ID NO: 8227 represents the cDNA sequence for clone AA847698.

SEQ ID NO: 8228 represents the cDNA sequence for clone AA847704.

SEQ ID NO: 8229 represents the cDNA sequence for clone AA847706.

SEQ ID NO: 8230 represents the cDNA sequence for clone AA847707.

SEQ ID NO: 8231 represents the cDNA sequence for clone AA847708.

SEQ ID NO: 8232 represents the cDNA sequence for clone AA847710.

SEQ ID NO: 8233 represents the cDNA sequence for clone AA847712.

SEQ ID NO: 8234 represents the cDNA sequence for clone AA847713.

SEQ ID NO: 8235 represents the cDNA sequence for clone AA847714.

SEQ ID NO: 8236 represents the cDNA sequence for clone AA847715.

SEQ ID NO: 8237 represents the cDNA sequence for clone AA847718.

SEQ ID NO: 8238 represents the cDNA sequence for clone AA847719.

SEQ ID NO: 8239 represents the cDNA sequence for clone AA847722.

SEQ ID NO: 8240 represents the cDNA sequence for clone AA847725.

SEQ ID NO: 8241 represents the cDNA sequence for clone AA847726.

SEQ ID NO: 8242 represents the cDNA sequence for clone AA847727.

SEQ ID NO: 8243 represents the cDNA sequence for clone AA847730.

SEQ ID NO: 8244 represents the cDNA sequence for clone AA847731.

SEQ ID NO: 8245 represents the cDNA sequence for clone AA847732.

SEQ ID NO: 8246 represents the cDNA sequence for clone AA847733.

SEQ ID NO: 8247 represents the cDNA sequence for clone AA847734.

SEQ ID NO: 8248 represents the cDNA sequence for clone AA847735.

SEQ ID NO: 8249 represents the cDNA sequence for clone AA847740.

SEQ ID NO: 8250 represents the cDNA sequence for clone AA847743.

SEQ ID NO: 8251 represents the cDNA sequence for clone AA847744.

SEQ ID NO: 8252 represents the cDNA sequence for clone AA847745.

SEQ ID NO: 8253 represents the cDNA sequence for clone AA847747.

SEQ ID NO: 8254 represents the cDNA sequence for clone AA847748.

SEQ ID NO: 8255 represents the cDNA sequence for clone AA847751.

SEQ ID NO: 8256 represents the cDNA sequence for clone AA847752.

SEQ ID NO: 8257 represents the cDNA sequence for clone AA847754.

SEQ ID NO: 8258 represents the cDNA sequence for clone AA847947.

SEQ ID NO: 8259 represents the cDNA sequence for clone AA847949.

SEQ ID NO: 8260 represents the cDNA sequence for clone AA847950.

SEQ ID NO: 8261 represents the cDNA sequence for clone AA847951.

SEQ ID NO: 8262 represents the cDNA sequence for clone AA847954.

SEQ ID NO: 8263 represents the cDNA sequence for clone AA847958.

SEQ ID NO: 8264 represents the cDNA sequence for clone AA847959.

SEQ ID NO: 8265 represents the cDNA sequence for clone AA847960.

SEQ ID NO: 8266 represents the cDNA sequence for clone AA847961.

SEQ ID NO: 8267 represents the cDNA sequence for clone AA847962.

SEQ ID NO: 8268 represents the cDNA sequence for clone AA847968.

SEQ ID NO: 8269 represents the cDNA sequence for clone AA847970.

SEQ ID NO: 8270 represents the cDNA sequence for clone AA847973.

SEQ ID NO: 8271 represents the cDNA sequence for clone AA847974.

SEQ ID NO: 8272 represents the cDNA sequence for clone AA847975.

SEQ ID NO: 8273 represents the cDNA sequence for clone AA847976.

SEQ ID NO: 8274 represents the cDNA sequence for clone AA847979.

SEQ ID NO: 8275 represents the cDNA sequence for clone AA847981.

SEQ ID NO: 8276 represents the cDNA sequence for clone AA847983.

SEQ ID NO: 8277 represents the cDNA sequence for clone AA847984.

SEQ ID NO: 8278 represents the cDNA sequence for clone AA847986.

SEQ ID NO: 8279 represents the cDNA sequence for clone AA847988.

SEQ ID NO: 8280 represents the cDNA sequence for clone AA847991.

SEQ ID NO: 8281 represents the cDNA sequence for clone AA847996.

SEQ ID NO: 8282 represents the cDNA sequence for clone AA847997.

SEQ ID NO: 8283 represents the cDNA sequence for clone AA847998.

SEQ ID NO: 8284 represents the cDNA sequence for clone AA847999.

SEQ ID NO: 8285 represents the cDNA sequence for clone AA848000.

SEQ ID NO: 8286 represents the cDNA sequence for clone AA848002.

SEQ ID NO: 8287 represents the cDNA sequence for clone AA848058.

SEQ ID NO: 8288 represents the cDNA sequence for clone AA848062.

SEQ ID NO: 8289 represents the cDNA sequence for clone AA848063.

SEQ ID NO: 8290 represents the cDNA sequence for clone AA848119.

SEQ ID NO: 8291 represents the cDNA sequence for clone AA848124.

SEQ ID NO: 8292 represents the cDNA sequence for clone AA848125.

SEQ ID NO: 8293 represents the cDNA sequence for clone AA848127.

SEQ ID NO: 8294 represents the cDNA sequence for clone AA848128.

SEQ ID NO: 8295 represents the cDNA sequence for clone AA848130.

SEQ ID NO: 8296 represents the cDNA sequence for clone AA848132.

SEQ ID NO: 8297 represents the cDNA sequence for clone AA848133.

SEQ ID NO: 8298 represents the cDNA sequence for clone AA848135.

SEQ ID NO: 8299 represents the cDNA sequence for clone AA848136.

SEQ ID NO: 8300 represents the cDNA sequence for clone AA848137.

SEQ ID NO: 8301 represents the cDNA sequence for clone AA848138.

SEQ ID NO: 8302 represents the cDNA sequence for clone AA848140.

SEQ ID NO: 8303 represents the cDNA sequence for clone AA848142.

SEQ ID NO: 8304 represents the cDNA sequence for clone AA848143.

SEQ ID NO: 8305 represents the cDNA sequence for clone AA848145.

SEQ ID NO: 8306 represents the cDNA sequence for clone AA848146.

SEQ ID NO: 8307 represents the cDNA sequence for clone AA848147.

SEQ ID NO: 8308 represents the cDNA sequence for clone AA848155.

SEQ ID NO: 8309 represents the cDNA sequence for clone AA848158.

SEQ ID NO: 8310 represents the cDNA sequence for clone AA848161.

SEQ ID NO: 8311 represents the cDNA sequence for clone AA848163.

SEQ ID NO: 8312 represents the cDNA sequence for clone AA848164.

SEQ ID NO: 8313 represents the cDNA sequence for clone AA848167.

SEQ ID NO: 8314 represents the cDNA sequence for clone AA848170.

SEQ ID NO: 8315 represents the cDNA sequence for clone AA848171.

SEQ ID NO: 8316 represents the cDNA sequence for clone AA848173.

SEQ ID NO: 8317 represents the cDNA sequence for clone AA848174.

SEQ ID NO: 8318 represents the cDNA sequence for clone AA848175.

SEQ ID NO: 8319 represents the cDNA sequence for clone AA856815.

SEQ ID NO: 8320 represents the cDNA sequence for clone AA856817.

SEQ ID NO: 8321 represents the cDNA sequence for clone AA856818.

SEQ ID NO: 8322 represents the cDNA sequence for clone AA856819.

SEQ ID NO: 8323 represents the cDNA sequence for clone AA856824.

SEQ ID NO: 8324 represents the cDNA sequence for clone AA856826.

SEQ ID NO: 8325 represents the cDNA sequence for clone AA856827.

SEQ ID NO: 8326 represents the cDNA sequence for clone AA856828.

SEQ ID NO: 8327 represents the cDNA sequence for clone AA856833.

SEQ ID NO: 8328 represents the cDNA sequence for clone AA856835.

SEQ ID NO: 8329 represents the cDNA sequence for clone AA856836.

SEQ ID NO: 8330 represents the cDNA sequence for clone AA856838.

SEQ ID NO: 8331 represents the cDNA sequence for clone AA856839.

SEQ ID NO: 8332 represents the cDNA sequence for clone AA856840.

SEQ ID NO: 8333 represents the cDNA sequence for clone AA856842.

SEQ ID NO: 8334 represents the cDNA sequence for clone AA856843.

SEQ ID NO: 8335 represents the cDNA sequence for clone AA856844.

SEQ ID NO: 8336 represents the cDNA sequence for clone AA856845.

SEQ ID NO: 8337 represents the cDNA sequence for clone AA856849.

SEQ ID NO: 8338 represents the cDNA sequence for clone AA856850.

SEQ ID NO: 8339 represents the cDNA sequence for clone AA856852.

SEQ ID NO: 8340 represents the cDNA sequence for clone AA856853.

SEQ ID NO: 8341 represents the cDNA sequence for clone AA856856.

SEQ ID NO: 8342 represents the cDNA sequence for clone AA856860.

SEQ ID NO: 8343 represents the cDNA sequence for clone AA856861.

SEQ ID NO: 8344 represents the cDNA sequence for clone AA856863.

SEQ ID NO: 8345 represents the cDNA sequence for clone AA856864.

SEQ ID NO: 8346 represents the cDNA sequence for clone AA856865.

SEQ ID NO: 8347 represents the cDNA sequence for clone AA856866.

SEQ ID NO: 8348 represents the cDNA sequence for clone AA856875.

SEQ ID NO: 8349 represents the cDNA sequence for clone AA856921.

SEQ ID NO: 8350 represents the cDNA sequence for clone AA856922.

SEQ ID NO: 8351 represents the cDNA sequence for clone AA856923.

SEQ ID NO: 8352 represents the cDNA sequence for clone AA856925.

SEQ ID NO: 8353 represents the cDNA sequence for clone AA856927.

SEQ ID NO: 8354 represents the cDNA sequence for clone AA856928.

SEQ ID NO: 8355 represents the cDNA sequence for clone AA856929.

SEQ ID NO: 8356 represents the cDNA sequence for clone AA856933.

SEQ ID NO: 8357 represents the cDNA sequence for clone AA856934.

SEQ ID NO: 8358 represents the cDNA sequence for clone AA856935.

SEQ ID NO: 8359 represents the cDNA sequence for clone AA856936.

SEQ ID NO: 8360 represents the cDNA sequence for clone AA856938.

SEQ ID NO: 8361 represents the cDNA sequence for clone AA856940.

SEQ ID NO: 8362 represents the cDNA sequence for clone AA856941.

SEQ ID NO: 8363 represents the cDNA sequence for clone AA856945.

SEQ ID NO: 8364 represents the cDNA sequence for clone AA856946.

SEQ ID NO: 8365 represents the cDNA sequence for clone AA856953.

SEQ ID NO: 8366 represents the cDNA sequence for clone AA856956.

SEQ ID NO: 8367 represents the cDNA sequence for clone AA856958.

SEQ ID NO: 8368 represents the cDNA sequence for clone AA856961.

SEQ ID NO: 8369 represents the cDNA sequence for clone AA856962.

SEQ ID NO: 8370 represents the cDNA sequence for clone AA856965.

SEQ ID NO: 8371 represents the cDNA sequence for clone AA856966.

SEQ ID NO: 8372 represents the cDNA sequence for clone AA856972.

SEQ ID NO: 8373 represents the cDNA sequence for clone AA856976.

SEQ ID NO: 8374 represents the cDNA sequence for clone AA856978.

SEQ ID NO: 8375 represents the cDNA sequence for clone AA856979.

SEQ ID NO: 8376 represents the cDNA sequence for clone AA856980.

SEQ ID NO: 8377 represents the cDNA sequence for clone AA856981.

SEQ ID NO: 8378 represents the cDNA sequence for clone AA857367.

SEQ ID NO: 8379 represents the cDNA sequence for clone AA857372.

SEQ ID NO: 8380 represents the cDNA sequence for clone AA857374.

SEQ ID NO: 8381 represents the cDNA sequence for clone AA857375.

SEQ ID NO: 8382 represents the cDNA sequence for clone AA857377.

SEQ ID NO: 8383 represents the cDNA sequence for clone AA857379.

SEQ ID NO: 8384 represents the cDNA sequence for clone AA857380.

SEQ ID NO: 8385 represents the cDNA sequence for clone AA857381.

SEQ ID NO: 8386 represents the cDNA sequence for clone AA857383.

SEQ ID NO: 8387 represents the cDNA sequence for clone AA857385.

SEQ ID NO: 8388 represents the cDNA sequence for clone AA857386.

SEQ ID NO: 8389 represents the cDNA sequence for clone AA857387.

SEQ ID NO: 8390 represents the cDNA sequence for clone AA857389.

SEQ ID NO: 8391 represents the cDNA sequence for clone AA857485.

SEQ ID NO: 8392 represents the cDNA sequence for clone AA857487.

SEQ ID NO: 8393 represents the cDNA sequence for clone AA857489.

SEQ ID NO: 8394 represents the cDNA sequence for clone AA857491.

SEQ ID NO: 8395 represents the cDNA sequence for clone AA857492.

SEQ ID NO: 8396 represents the cDNA sequence for clone AA857493.

SEQ ID NO: 8397 represents the cDNA sequence for clone AA857494.

SEQ ID NO: 8398 represents the cDNA sequence for clone AA857496.

SEQ ID NO: 8399 represents the cDNA sequence for clone AA857501.

SEQ ID NO: 8400 represents the cDNA sequence for clone AA857503.

SEQ ID NO: 8401 represents the cDNA sequence for clone AA862175.

SEQ ID NO: 8402 represents the cDNA sequence for clone AA862176.

SEQ ID NO: 8403 represents the cDNA sequence for clone AA862178.

SEQ ID NO: 8404 represents the cDNA sequence for clone AA862181.

SEQ ID NO: 8405 represents the cDNA sequence for clone AA862183.

SEQ ID NO: 8406 represents the cDNA sequence for clone AA862185.

SEQ ID NO: 8407 represents the cDNA sequence for clone AA862188.

SEQ ID NO: 8408 represents the cDNA sequence for clone AA862189.

SEQ ID NO: 8409 represents the cDNA sequence for clone AA862191.

SEQ ID NO: 8410 represents the cDNA sequence for clone AA862192.

SEQ ID NO: 8411 represents the cDNA sequence for clone AA862193.

SEQ ID NO: 8412 represents the cDNA sequence for clone AA862194.

SEQ ID NO: 8413 represents the cDNA sequence for clone AA862195.

SEQ ID NO: 8414 represents the cDNA sequence for clone AA862196.

SEQ ID NO: 8415 represents the cDNA sequence for clone AA862197.

SEQ ID NO: 8416 represents the cDNA sequence for clone AA862199.

SEQ ID NO: 8417 represents the cDNA sequence for clone AA862201.

SEQ ID NO: 8418 represents the cDNA sequence for clone AA862204.

SEQ ID NO: 8419 represents the cDNA sequence for clone AA862208.

SEQ ID NO: 8420 represents the cDNA sequence for clone AA862211.

SEQ ID NO: 8421 represents the cDNA sequence for clone AA862212.

SEQ ID NO: 8422 represents the cDNA sequence for clone AA862214.

SEQ ID NO: 8423 represents the cDNA sequence for clone AA862215.

SEQ ID NO: 8424 represents the cDNA sequence for clone AA862216.

SEQ ID NO: 8425 represents the cDNA sequence for clone AA862217.

SEQ ID NO: 8426 represents the cDNA sequence for clone AA862218.

SEQ ID NO: 8427 represents the cDNA sequence for clone AA862219.

SEQ ID NO: 8428 represents the cDNA sequence for clone AA862220.

SEQ ID NO: 8429 represents the cDNA sequence for clone AA862221.

SEQ ID NO: 8430 represents the cDNA sequence for clone AA862223.

SEQ ID NO: 8431 represents the cDNA sequence for clone AA862224.

SEQ ID NO: 8432 represents the cDNA sequence for clone AA862225.

SEQ ID NO: 8433 represents the cDNA sequence for clone AA862226.

SEQ ID NO: 8434 represents the cDNA sequence for clone AA862228.

SEQ ID NO: 8435 represents the cDNA sequence for clone AA862229.

SEQ ID NO: 8436 represents the cDNA sequence for clone AA862230.

SEQ ID NO: 8437 represents the cDNA sequence for clone AA862235.

SEQ ID NO: 8438 represents the cDNA sequence for clone AA862238.

SEQ ID NO: 8439 represents the cDNA sequence for clone AA862239.

SEQ ID NO: 8440 represents the cDNA sequence for clone AA862241.

SEQ ID NO: 8441 represents the cDNA sequence for clone AA862242.

SEQ ID NO: 8442 represents the cDNA sequence for clone AA862249.

SEQ ID NO: 8443 represents the cDNA sequence for clone AA873651.

SEQ ID NO: 8444 represents the cDNA sequence for clone AA873652.

SEQ ID NO: 8445 represents the cDNA sequence for clone AA873654.

SEQ ID NO: 8446 represents the cDNA sequence for clone AA873664.

SEQ ID NO: 8447 represents the cDNA sequence for clone AA873665.

SEQ ID NO: 8448 represents the cDNA sequence for clone AA873666.

SEQ ID NO: 8449 represents the cDNA sequence for clone AA873667.

SEQ ID NO: 8450 represents the cDNA sequence for clone AA873670.

SEQ ID NO: 8451 represents the cDNA sequence for clone AA873671.

SEQ ID NO: 8452 represents the cDNA sequence for clone AA873672.

SEQ ID NO: 8453 represents the cDNA sequence for clone AA878332.

SEQ ID NO: 8454 represents the cDNA sequence for clone AA878337.

SEQ ID NO: 8455 represents the cDNA sequence for clone AA878343.

SEQ ID NO: 8456 represents the cDNA sequence for clone AA878346.

SEQ ID NO: 8457 represents the cDNA sequence for clone AA878348.

SEQ ID NO: 8458 represents the cDNA sequence for clone AA878349.

SEQ ID NO: 8459 represents the cDNA sequence for clone AA878350.

SEQ ID NO: 8460 represents the cDNA sequence for clone AA878351.

SEQ ID NO: 8461 represents the cDNA sequence for clone AA878354.

SEQ ID NO: 8462 represents the cDNA sequence for clone AA878356.

SEQ ID NO: 8463 represents the cDNA sequence for clone AA878357.

SEQ ID NO: 8464 represents the cDNA sequence for clone AA878358.

SEQ ID NO: 8465 represents the cDNA sequence for clone AA878359.

SEQ ID NO: 8466 represents the cDNA sequence for clone AA878362.

SEQ ID NO: 8467 represents the cDNA sequence for clone AA878403.

SEQ ID NO: 8468 represents the cDNA sequence for clone AA878407.

SEQ ID NO: 8469 represents the cDNA sequence for clone AA878410.

SEQ ID NO: 8470 represents the cDNA sequence for clone AA878413.

SEQ ID NO: 8471 represents the cDNA sequence for clone AA878415.

SEQ ID NO: 8472 represents the cDNA sequence for clone AA878416.

SEQ ID NO: 8473 represents the cDNA sequence for clone AA878418.

SEQ ID NO: 8474 represents the cDNA sequence for clone AA878419.

SEQ ID NO: 8475 represents the cDNA sequence for clone AA878420.

SEQ ID NO: 8476 represents the cDNA sequence for clone AA878423.

SEQ ID NO: 8477 represents the cDNA sequence for clone AA878424.

SEQ ID NO: 8478 represents the cDNA sequence for clone AA878433.

SEQ ID NO: 8479 represents the cDNA sequence for clone AA878436.

SEQ ID NO: 8480 represents the cDNA sequence for clone AA878439.

SEQ ID NO: 8481 represents the cDNA sequence for clone AA878440.

SEQ ID NO: 8482 represents the cDNA sequence for clone AA878442.

SEQ ID NO: 8483 represents the cDNA sequence for clone AA878484.

SEQ ID NO: 8484 represents the cDNA sequence for clone AA878487.

SEQ ID NO: 8485 represents the cDNA sequence for clone AA878491.

SEQ ID NO: 8486 represents the cDNA sequence for clone AA878493.

SEQ ID NO: 8487 represents the cDNA sequence for clone AA878495.

SEQ ID NO: 8488 represents the cDNA sequence for clone AA878498.

SEQ ID NO: 8489 represents the cDNA sequence for clone AA878499.

SEQ ID NO: 8490 represents the cDNA sequence for clone AA878502.

SEQ ID NO: 8491 represents the cDNA sequence for clone AA902676.

SEQ ID NO: 8492 represents the cDNA sequence for clone AA909822.

SEQ ID NO: 8493 represents the cDNA sequence for clone AA909830.

SEQ ID NO: 8494 represents the cDNA sequence for clone AA908312.

SEQ ID NO: 8495 represents the cDNA sequence for clone AA908317.

SEQ ID NO: 8496 represents the cDNA sequence for clone AA908323.

SEQ ID NO: 8497 represents the cDNA sequence for clone AA908324.

SEQ ID NO: 8498 represents the cDNA sequence for clone AA908328.

SEQ ID NO: 8499 represents the cDNA sequence for clone AA908332.

SEQ ID NO: 8500 represents the cDNA sequence for clone AA908339.

SEQ ID NO: 8501 represents the cDNA sequence for clone AA908348.

SEQ ID NO: 8502 represents the cDNA sequence for clone AA908349.

SEQ ID NO: 8503 represents the cDNA sequence for clone AA908351.

SEQ ID NO: 8504 represents the cDNA sequence for clone AA908354.

SEQ ID NO: 8505 represents the cDNA sequence for clone AA908361.

SEQ ID NO: 8506 represents the cDNA sequence for clone AA908377.

SEQ ID NO: 8507 represents the cDNA sequence for clone AA908383.

SEQ ID NO: 8508 represents the cDNA sequence for clone AA908389.

SEQ ID NO: 8509 represents the cDNA sequence for clone AA908390.

SEQ ID NO: 8510 represents the cDNA sequence for clone AA908396.

SEQ ID NO: 8511 represents the cDNA sequence for clone AA908400.

SEQ ID NO: 8512 represents the cDNA sequence for clone AA908404.

SEQ ID NO: 8513 represents the cDNA sequence for clone AA908407.

SEQ ID NO: 8514 represents the cDNA sequence for clone AA908415.

SEQ ID NO: 8515 represents the cDNA sequence for clone AA908417.

SEQ ID NO: 8516 represents the cDNA sequence for clone AA908419.

SEQ ID NO: 8517 represents the cDNA sequence for clone AA908431.

SEQ ID NO: 8518 represents the cDNA sequence for clone AA908433.

SEQ ID NO: 8519 represents the cDNA sequence for clone AA908440.

SEQ ID NO: 8520 represents the cDNA sequence for clone AA908452.

SEQ ID NO: 8521 represents the cDNA sequence for clone AA908470.

SEQ ID NO: 8522 represents the cDNA sequence for clone AA908474.

SEQ ID NO: 8523 represents the cDNA sequence for clone AA908491.

SEQ ID NO: 8524 represents the cDNA sequence for clone AA908494.

SEQ ID NO: 8525 represents the cDNA sequence for clone AA908496.

SEQ ID NO: 8526 represents the cDNA sequence for clone AA908499.

SEQ ID NO: 8527 represents the cDNA sequence for clone AA908516.

SEQ ID NO: 8528 represents the cDNA sequence for clone AA908532.

SEQ ID NO: 8529 represents the cDNA sequence for clone AA908537.

SEQ ID NO: 8530 represents the cDNA sequence for clone AA908554.

SEQ ID NO: 8531 represents the cDNA sequence for clone AA908555.

SEQ ID NO: 8532 represents the cDNA sequence for clone AA908560.

SEQ ID NO: 8533 represents the cDNA sequence for clone AA908561.

SEQ ID NO: 8534 represents the cDNA sequence for clone AA908563.

SEQ ID NO: 8535 represents the cDNA sequence for clone AA908567.

SEQ ID NO: 8536 represents the cDNA sequence for clone AA908576.

SEQ ID NO: 8537 represents the cDNA sequence for clone AA908580.

SEQ ID NO: 8538 represents the cDNA sequence for clone AA908582.

SEQ ID NO: 8539 represents the cDNA sequence for clone AA908592.

SEQ ID NO: 8540 represents the cDNA sequence for clone AA908593.

SEQ ID NO: 8541 represents the cDNA sequence for clone AA908602.

SEQ ID NO: 8542 represents the cDNA sequence for clone AA908603.

SEQ ID NO: 8543 represents the cDNA sequence for clone AA908604.

SEQ ID NO: 8544 represents the cDNA sequence for clone AA908606.

SEQ ID NO: 8545 represents the cDNA sequence for clone AA908607.

SEQ ID NO: 8546 represents the cDNA sequence for clone AA908610.

SEQ ID NO: 8547 represents the cDNA sequence for clone AA908614.

SEQ ID NO: 8548 represents the cDNA sequence for clone AA908638.

SEQ ID NO: 8549 represents the cDNA sequence for clone AA908733.

SEQ ID NO: 8550 represents the cDNA sequence for clone AA908738.

SEQ ID NO: 8551 represents the cDNA sequence for clone AA908739.

SEQ ID NO: 8552 represents the cDNA sequence for clone AA908744.

SEQ ID NO: 8553 represents the cDNA sequence for clone AA908747.

SEQ ID NO: 8554 represents the cDNA sequence for clone AA908753.

SEQ ID NO: 8555 represents the cDNA sequence for clone AA908756.

SEQ ID NO: 8556 represents the cDNA sequence for clone AA908805.

SEQ ID NO: 8557 represents the cDNA sequence for clone AA908806.

SEQ ID NO: 8558 represents the cDNA sequence for clone AA908810.

SEQ ID NO: 8559 represents the cDNA sequence for clone AA908812.

SEQ ID NO: 8560 represents the cDNA sequence for clone AA908813.

SEQ ID NO: 8561 represents the cDNA sequence for clone AA908819.

SEQ ID NO: 8562 represents the cDNA sequence for clone AA910288.

SEQ ID NO: 8563 represents the cDNA sequence for clone AA910290.

SEQ ID NO: 8564 represents the cDNA sequence for clone AA937561.

SEQ ID NO: 8565 represents the cDNA sequence for clone AA937572.

SEQ ID NO: 8566 represents the cDNA sequence for clone AA937574.

SEQ ID NO: 8567 represents the cDNA sequence for clone AA948725.

SEQ ID NO: 8568 represents the cDNA sequence for clone AA948726.

SEQ ID NO: 8569 represents the cDNA sequence for clone AA948727.

SEQ ID NO: 8570 represents the cDNA sequence for clone AA948731.

SEQ ID NO: 8571 represents the cDNA sequence for clone AA948732.

SEQ ID NO: 8572 represents the cDNA sequence for clone AA948735.

SEQ ID NO: 8573 represents the cDNA sequence for clone AA948736.

SEQ ID NO: 8574 represents the cDNA sequence for clone AA948738.

SEQ ID NO: 8575 represents the cDNA sequence for clone AA947254.

SEQ ID NO: 8576 represents the cDNA sequence for clone AA947255.

SEQ ID NO: 8577 represents the cDNA sequence for clone AA947257.

SEQ ID NO: 8578 represents the cDNA sequence for clone AA947258.

SEQ ID NO: 8579 represents the cDNA sequence for clone AA947259.

SEQ ID NO: 8580 represents the cDNA sequence for clone AA947262.

SEQ ID NO: 8581 represents the cDNA sequence for clone AA947264.

SEQ ID NO: 8582 represents the cDNA sequence for clone AA947265.

SEQ ID NO: 8583 represents the cDNA sequence for clone AA947266.

SEQ ID NO: 8584 represents the cDNA sequence for clone AA947348.

SEQ ID NO: 8585 represents the cDNA sequence for clone AA947349.

SEQ ID NO: 8586 represents the cDNA sequence for clone AA947351.

SEQ ID NO: 8587 represents the cDNA sequence for clone AA947353.

SEQ ID NO: 8588 represents the cDNA sequence for clone AA947354.

SEQ ID NO: 8589 represents the cDNA sequence for clone AA947356.

SEQ ID NO: 8590 represents the cDNA sequence for clone AA947366.

SEQ ID NO: 8591 represents the cDNA sequence for clone AA947367.

SEQ ID NO: 8592 represents the cDNA sequence for clone AA947368.

SEQ ID NO: 8593 represents the cDNA sequence for clone AA947371.

SEQ ID NO: 8594 represents the cDNA sequence for clone AA947374.

SEQ ID NO: 8595 represents the cDNA sequence for clone AA947375.

SEQ ID NO: 8596 represents the cDNA sequence for clone AA947383.

SEQ ID NO: 8597 represents the cDNA sequence for clone AA947384.

SEQ ID NO: 8598 represents the cDNA sequence for clone AA947385.

SEQ ID NO: 8599 represents the cDNA sequence for clone AA947386.

SEQ ID NO: 8600 represents the cDNA sequence for clone AA947388.

SEQ ID NO: 8601 represents the cDNA sequence for clone AA947526.

SEQ ID NO: 8602 represents the cDNA sequence for clone AA947527.

SEQ ID NO: 8603 represents the cDNA sequence for clone AA947528.

SEQ ID NO: 8604 represents the cDNA sequence for clone AA947529.

SEQ ID NO: 8605 represents the cDNA sequence for clone AA947531.

SEQ ID NO: 8606 represents the cDNA sequence for clone AA947532.

SEQ ID NO: 8607 represents the cDNA sequence for clone AA947533.

SEQ ID NO: 8608 represents the cDNA sequence for clone AA947536.

SEQ ID NO: 8609 represents the cDNA sequence for clone AA947538.

SEQ ID NO: 8610 represents the cDNA sequence for clone AA947539.

SEQ ID NO: 8611 represents the cDNA sequence for clone AA947548.

SEQ ID NO: 8612 represents the cDNA sequence for clone AA947550.

SEQ ID NO: 8613 represents the cDNA sequence for clone AA947552.

SEQ ID NO: 8614 represents the cDNA sequence for clone AA947558.

SEQ ID NO: 8615 represents the cDNA sequence for clone AA947562.

SEQ ID NO: 8616 represents the cDNA sequence for clone AA947563.

SEQ ID NO: 8617 represents the cDNA sequence for clone AA947564.

SEQ ID NO: 8618 represents the cDNA sequence for clone AA947565.

SEQ ID NO: 8619 represents the cDNA sequence for clone AA947799.

SEQ ID NO: 8620 represents the cDNA sequence for clone AA947801.

SEQ ID NO: 8621 represents the cDNA sequence for clone AA947802.

SEQ ID NO: 8622 represents the cDNA sequence for clone AA947803.

SEQ ID NO: 8623 represents the cDNA sequence for clone AA947804.

SEQ ID NO: 8624 represents the cDNA sequence for clone AA947807.

SEQ ID NO: 8625 represents the cDNA sequence for clone AA947808.

SEQ ID NO: 8626 represents the cDNA sequence for clone AA947810.

SEQ ID NO: 8627 represents the cDNA sequence for clone AA947812.

SEQ ID NO: 8628 represents the cDNA sequence for clone AA947813.

SEQ ID NO: 8629 represents the cDNA sequence for clone AA947816.

SEQ ID NO: 8630 represents the cDNA sequence for clone AA947817.

SEQ ID NO: 8631 represents the cDNA sequence for clone AA947818.

SEQ ID NO: 8632 represents the cDNA sequence for clone AA947819.

SEQ ID NO: 8633 represents the cDNA sequence for clone AA947824.

SEQ ID NO: 8634 represents the cDNA sequence for clone AA947826.

SEQ ID NO: 8635 represents the cDNA sequence for clone AA947827.

SEQ ID NO: 8636 represents the cDNA sequence for clone AA947828.

SEQ ID NO: 8637 represents the cDNA sequence for clone AA947830.

SEQ ID NO: 8638 represents the cDNA sequence for clone AA947831.

SEQ ID NO: 8639 represents the cDNA sequence for clone AA947834.

SEQ ID NO: 8640 represents the cDNA sequence for clone AA947837.

SEQ ID NO: 8641 represents the cDNA sequence for clone AI053400.

SEQ ID NO: 8642 represents the cDNA sequence for clone AI053406.

SEQ ID NO: 8643 represents the cDNA sequence for clone AI053407.

SEQ ID NO: 8644 represents the cDNA sequence for clone AI053410.

SEQ ID NO: 8645 represents the cDNA sequence for clone AI053428.

SEQ ID NO: 8646 represents the cDNA sequence for clone AI053430.

SEQ ID NO: 8647 represents the cDNA sequence for clone AI053431.

SEQ ID NO: 8648 represents the cDNA sequence for clone AI053433.

SEQ ID NO: 8649 represents the cDNA sequence for clone AI053434.

SEQ ID NO: 8650 represents the cDNA sequence for clone AI053438.

SEQ ID NO: 8651 represents the cDNA sequence for clone AI053439.

SEQ ID NO: 8652 represents the cDNA sequence for clone AI053441.

SEQ ID NO: 8653 represents the cDNA sequence for clone AI053443.

SEQ ID NO: 8654 represents the cDNA sequence for clone AI053446.

SEQ ID NO: 8655 represents the cDNA sequence for clone AI053449.

SEQ ID NO: 8656 represents the cDNA sequence for clone AI053452.

SEQ ID NO: 8657 represents the cDNA sequence for clone AI053455.

SEQ ID NO: 8658 represents the cDNA sequence for clone AI053461.

SEQ ID NO: 8659 represents the cDNA sequence for clone AI053462.

SEQ ID NO: 8660 represents the cDNA sequence for clone AI053466.

SEQ ID NO: 8661 represents the cDNA sequence for clone AI053469.

SEQ ID NO: 8662 represents the cDNA sequence for clone AI053472.

SEQ ID NO: 8663 represents the cDNA sequence for clone AI053480.

SEQ ID NO: 8664 represents the cDNA sequence for clone AI053481.

SEQ ID NO: 8665 represents the cDNA sequence for clone AI053482.

SEQ ID NO: 8666 represents the cDNA sequence for clone AI053484.

SEQ ID NO: 8667 represents the cDNA sequence for clone AI053485.

SEQ ID NO: 8668 represents the cDNA sequence for clone AI053486.

SEQ ID NO: 8669 represents the cDNA sequence for clone AI053489.

SEQ ID NO: 8670 represents the cDNA sequence for clone AI053492.

SEQ ID NO: 8671 represents the cDNA sequence for clone AI053496.

SEQ ID NO: 8672 represents the cDNA sequence for clone AI053501.

SEQ ID NO: 8673 represents the cDNA sequence for clone AI053503.

SEQ ID NO: 8674 represents the cDNA sequence for clone AI053514.

SEQ ID NO: 8675 represents the cDNA sequence for clone AI053519.

SEQ ID NO: 8676 represents the cDNA sequence for clone AI053520.

SEQ ID NO: 8677 represents the cDNA sequence for clone AI053529.

SEQ ID NO: 8678 represents the cDNA sequence for clone AI053536.

SEQ ID NO: 8679 represents the cDNA sequence for clone AI053538.

SEQ ID NO: 8680 represents the cDNA sequence for clone AI053540.

SEQ ID NO: 8681 represents the cDNA sequence for clone AI053543.

SEQ ID NO: 8682 represents the cDNA sequence for clone AI053545.

SEQ ID NO: 8683 represents the cDNA sequence for clone AI053547.

SEQ ID NO: 8684 represents the cDNA sequence for clone AI053551.

SEQ ID NO: 8685 represents the cDNA sequence for clone AI053552.

SEQ ID NO: 8686 represents the cDNA sequence for clone AI053555.

SEQ ID NO: 8687 represents the cDNA sequence for clone AI053568.

SEQ ID NO: 8688 represents the cDNA sequence for clone AI053570.

SEQ ID NO: 8689 represents the cDNA sequence for clone AI053572.

SEQ ID NO: 8690 represents the cDNA sequence for clone AI053582.

SEQ ID NO: 8691 represents the cDNA sequence for clone AI053585.

SEQ ID NO: 8692 represents the cDNA sequence for clone AI053586.

SEQ ID NO: 8693 represents the cDNA sequence for clone AI053588.

SEQ ID NO: 8694 represents the cDNA sequence for clone AI053589.

SEQ ID NO: 8695 represents the cDNA sequence for clone AI053590.

SEQ ID NO: 8696 represents the cDNA sequence for clone AI053591.

SEQ ID NO: 8697 represents the cDNA sequence for clone AI053594.

SEQ ID NO: 8698 represents the cDNA sequence for clone AI053595.

SEQ ID NO: 8699 represents the cDNA sequence for clone AI053596.

SEQ ID NO: 8700 represents the cDNA sequence for clone AI053597.

SEQ ID NO: 8701 represents the cDNA sequence for clone AI053600.

SEQ ID NO: 8702 represents the cDNA sequence for clone AI053602.

SEQ ID NO: 8703 represents the cDNA sequence for clone AI053605.

SEQ ID NO: 8704 represents the cDNA sequence for clone AI053612.

SEQ ID NO: 8705 represents the cDNA sequence for clone AI053613.

SEQ ID NO: 8706 represents the cDNA sequence for clone AI053614.

SEQ ID NO: 8707 represents the cDNA sequence for clone AI053617.

SEQ ID NO: 8708 represents the cDNA sequence for clone AI053619.

SEQ ID NO: 8709 represents the cDNA sequence for clone AI053622.

SEQ ID NO: 8710 represents the cDNA sequence for clone AI053623.

SEQ ID NO: 8711 represents the cDNA sequence for clone AI053625.

SEQ ID NO: 8712 represents the cDNA sequence for clone AI053629.

SEQ ID NO: 8713 represents the cDNA sequence for clone AI053640.

SEQ ID NO: 8714 represents the cDNA sequence for clone AI053641.

SEQ ID NO: 8715 represents the cDNA sequence for clone AI053643.

SEQ ID NO: 8716 represents the cDNA sequence for clone AI053645.

SEQ ID NO: 8717 represents the cDNA sequence for clone AI053647.

SEQ ID NO: 8718 represents the cDNA sequence for clone AI053650.

SEQ ID NO: 8719 represents the cDNA sequence for clone AI053651.

SEQ ID NO: 8720 represents the cDNA sequence for clone AI053653.

SEQ ID NO: 8721 represents the cDNA sequence for clone AI053659.

SEQ ID NO: 8722 represents the cDNA sequence for clone AI053662.

SEQ ID NO: 8723 represents the cDNA sequence for clone AI053663.

SEQ ID NO: 8724 represents the cDNA sequence for clone AI053674.

SEQ ID NO: 8725 represents the cDNA sequence for clone AI053684.

SEQ ID NO: 8726 represents the cDNA sequence for clone AI053694.

SEQ ID NO: 8727 represents the cDNA sequence for clone AI053707.

SEQ ID NO: 8728 represents the cDNA sequence for clone AI053710.

SEQ ID NO: 8729 represents the cDNA sequence for clone AI053711.

SEQ ID NO: 8730 represents the cDNA sequence for clone AI053713.

SEQ ID NO: 8731 represents the cDNA sequence for clone AI053715.

SEQ ID NO: 8732 represents the cDNA sequence for clone AI053719.

SEQ ID NO: 8733 represents the cDNA sequence for clone AI053725.

SEQ ID NO: 8734 represents the cDNA sequence for clone AI053729.

SEQ ID NO: 8735 represents the cDNA sequence for clone AI053731.

SEQ ID NO: 8736 represents the cDNA sequence for clone AI053738.

SEQ ID NO: 8737 represents the cDNA sequence for clone AI053741.

SEQ ID NO: 8738 represents the cDNA sequence for clone AI053743.

SEQ ID NO: 8739 represents the cDNA sequence for clone AI053745.

SEQ ID NO: 8740 represents the cDNA sequence for clone AI053751.

SEQ ID NO: 8741 represents the cDNA sequence for clone AI053753.

SEQ ID NO: 8742 represents the cDNA sequence for clone AI053755.

SEQ ID NO: 8743 represents the cDNA sequence for clone AI053759.

SEQ ID NO: 8744 represents the cDNA sequence for clone AI053763.

SEQ ID NO: 8745 represents the cDNA sequence for clone AI053764.

SEQ ID NO: 8746 represents the cDNA sequence for clone AI053765.

SEQ ID NO: 8747 represents the cDNA sequence for clone AI053768.

SEQ ID NO: 8748 represents the cDNA sequence for clone AI053769.

SEQ ID NO: 8749 represents the cDNA sequence for clone AI053771.

SEQ ID NO: 8750 represents the cDNA sequence for clone AI053773.

SEQ ID NO: 8751 represents the cDNA sequence for clone AI053776.

SEQ ID NO: 8752 represents the cDNA sequence for clone AI053777.

SEQ ID NO: 8753 represents the cDNA sequence for clone AI053778.

SEQ ID NO: 8754 represents the cDNA sequence for clone AI053779.

SEQ ID NO: 8755 represents the cDNA sequence for clone AI053780.

SEQ ID NO: 8756 represents the cDNA sequence for clone AI053784.

SEQ ID NO: 8757 represents the cDNA sequence for clone AI053786.

SEQ ID NO: 8758 represents the cDNA sequence for clone AI053788.

SEQ ID NO: 8759 represents the cDNA sequence for clone AI053791.

SEQ ID NO: 8760 represents the cDNA sequence for clone AI053795.

SEQ ID NO: 8761 represents the cDNA sequence for clone AI053796.

SEQ ID NO: 8762 represents the cDNA sequence for clone AI053797.

SEQ ID NO: 8763 represents the cDNA sequence for clone AI053799.

SEQ ID NO: 8764 represents the cDNA sequence for clone AI053800.

SEQ ID NO: 8765 represents the cDNA sequence for clone AI053801.

SEQ ID NO: 8766 represents the cDNA sequence for clone AI053802.

SEQ ID NO: 8767 represents the cDNA sequence for clone AI053806.

SEQ ID NO: 8768 represents the cDNA sequence for clone AI053807.

SEQ ID NO: 8769 represents the cDNA sequence for clone AI053808.

SEQ ID NO: 8770 represents the cDNA sequence for clone AI053816.

SEQ ID NO: 8771 represents the cDNA sequence for clone AI053820.

SEQ ID NO: 8772 represents the cDNA sequence for clone AI053831.

SEQ ID NO: 8773 represents the cDNA sequence for clone AI053834.

SEQ ID NO: 8774 represents the cDNA sequence for clone AI053836.

SEQ ID NO: 8775 represents the cDNA sequence for clone AI053837.

SEQ ID NO: 8776 represents the cDNA sequence for clone AI053841.

SEQ ID NO: 8777 represents the cDNA sequence for clone AI053842.

SEQ ID NO: 8778 represents the cDNA sequence for clone AI053846.

SEQ ID NO: 8779 represents the cDNA sequence for clone AI053848.

SEQ ID NO: 8780 represents the cDNA sequence for clone AI053851.

SEQ ID NO: 8781 represents the cDNA sequence for clone AI053852.

SEQ ID NO: 8782 represents the cDNA sequence for clone AI053853.

SEQ ID NO: 8783 represents the cDNA sequence for clone AI053860.

SEQ ID NO: 8784 represents the cDNA sequence for clone AI053862.

SEQ ID NO: 8785 represents the cDNA sequence for clone AI053866.

SEQ ID NO: 8786 represents the cDNA sequence for clone AI053870.

SEQ ID NO: 8787 represents the cDNA sequence for clone AI053871.

SEQ ID NO: 8788 represents the cDNA sequence for clone AI053874.

SEQ ID NO: 8789 represents the cDNA sequence for clone AI053879.

SEQ ID NO: 8790 represents the cDNA sequence for clone AI053882.

SEQ ID NO: 8791 represents the cDNA sequence for clone AI053883.

SEQ ID NO: 8792 represents the cDNA sequence for clone AI053884.

SEQ ID NO: 8793 represents the cDNA sequence for clone AI053890.

SEQ ID NO: 8794 represents the cDNA sequence for clone AI053891.

SEQ ID NO: 8795 represents the cDNA sequence for clone AI053893.

SEQ ID NO: 8796 represents the cDNA sequence for clone AI053895.

SEQ ID NO: 8797 represents the cDNA sequence for clone AI053898.

SEQ ID NO: 8798 represents the cDNA sequence for clone AI053905.

SEQ ID NO: 8799 represents the cDNA sequence for clone AI053908.

SEQ ID NO: 8800 represents the cDNA sequence for clone AI053910.

SEQ ID NO: 8801 represents the cDNA sequence for clone AI053912.

SEQ ID NO: 8802 represents the cDNA sequence for clone AI053918.

SEQ ID NO: 8803 represents the cDNA sequence for clone AI053934.

SEQ ID NO: 8804 represents the cDNA sequence for clone AI053935.

SEQ ID NO: 8805 represents the cDNA sequence for clone AI053936.

SEQ ID NO: 8806 represents the cDNA sequence for clone AI053940.

SEQ ID NO: 8807 represents the cDNA sequence for clone AI053941.

SEQ ID NO: 8808 represents the cDNA sequence for clone AI053942.

SEQ ID NO: 8809 represents the cDNA sequence for clone AI053946.

SEQ ID NO: 8810 represents the cDNA sequence for clone AI053949.

SEQ ID NO: 8811 represents the cDNA sequence for clone AI053950.

SEQ ID NO: 8812 represents the cDNA sequence for clone AI053951.

SEQ ID NO: 8813 represents the cDNA sequence for clone AI053952.

SEQ ID NO: 8814 represents the cDNA sequence for clone AI053955.

SEQ ID NO: 8815 represents the cDNA sequence for clone AI053961.

SEQ ID NO: 8816 represents the cDNA sequence for clone AI053963.

SEQ ID NO: 8817 represents the cDNA sequence for clone AI053964.

SEQ ID NO: 8818 represents the cDNA sequence for clone AI053970.

SEQ ID NO: 8819 represents the cDNA sequence for clone AI053972.

SEQ ID NO: 8820 represents the cDNA sequence for clone AI053974.

SEQ ID NO: 8821 represents the cDNA sequence for clone AI053978.

SEQ ID NO: 8822 represents the cDNA sequence for clone AI053983.

SEQ ID NO: 8823 represents the cDNA sequence for clone AI053987.

SEQ ID NO: 8824 represents the cDNA sequence for clone AI053992.

SEQ ID NO: 8825 represents the cDNA sequence for clone AI053994.

SEQ ID NO: 8826 represents the cDNA sequence for clone AI054003.

SEQ ID NO: 8827 represents the cDNA sequence for clone AI054011.

SEQ ID NO: 8828 represents the cDNA sequence for clone AI054014.

SEQ ID NO: 8829 represents the cDNA sequence for clone AI054019.

SEQ ID NO: 8830 represents the cDNA sequence for clone AI054020.

SEQ ID NO: 8831 represents the cDNA sequence for clone AI054023.

SEQ ID NO: 8832 represents the cDNA sequence for clone AI054024.

SEQ ID NO: 8833 represents the cDNA sequence for clone AI054032.

SEQ ID NO: 8834 represents the cDNA sequence for clone AI054036.

SEQ ID NO: 8835 represents the cDNA sequence for clone AI054038.

SEQ ID NO: 8836 represents the cDNA sequence for clone AI054039.

SEQ ID NO: 8837 represents the cDNA sequence for clone AI054041.

SEQ ID NO: 8838 represents the cDNA sequence for clone AI054046.

SEQ ID NO: 8839 represents the cDNA sequence for clone AI054048.

SEQ ID NO: 8840 represents the cDNA sequence for clone AI054052.

SEQ ID NO: 8841 represents the cDNA sequence for clone AI054055.

SEQ ID NO: 8842 represents the cDNA sequence for clone AI054056.

SEQ ID NO: 8843 represents the cDNA sequence for clone AI054058.

SEQ ID NO: 8844 represents the cDNA sequence for clone AI054068.

SEQ ID NO: 8845 represents the cDNA sequence for clone AI054069.

SEQ ID NO: 8846 represents the cDNA sequence for clone AI054074.

SEQ ID NO: 8847 represents the cDNA sequence for clone AI054081.

SEQ ID NO: 8848 represents the cDNA sequence for clone AI054084.

SEQ ID NO: 8849 represents the cDNA sequence for clone AI054087.

SEQ ID NO: 8850 represents the cDNA sequence for clone AI054091.

SEQ ID NO: 8851 represents the cDNA sequence for clone AI054093.

SEQ ID NO: 8852 represents the cDNA sequence for clone AI054095.

SEQ ID NO: 8853 represents the cDNA sequence for clone AI054096.

SEQ ID NO: 8854 represents the cDNA sequence for clone AI054102.

SEQ ID NO: 8855 represents the cDNA sequence for clone AI054103.

SEQ ID NO: 8856 represents the cDNA sequence for clone AI054105.

SEQ ID NO: 8857 represents the cDNA sequence for clone AI054106.

SEQ ID NO: 8858 represents the cDNA sequence for clone AI054107.

SEQ ID NO: 8859 represents the cDNA sequence for clone AI054108.

SEQ ID NO: 8860 represents the cDNA sequence for clone AI054114.

SEQ ID NO: 8861 represents the cDNA sequence for clone AI054120.

SEQ ID NO: 8862 represents the cDNA sequence for clone AI054127.

SEQ ID NO: 8863 represents the cDNA sequence for clone AI054131.

SEQ ID NO: 8864 represents the cDNA sequence for clone AI054134.

SEQ ID NO: 8865 represents the cDNA sequence for clone AI054141.

SEQ ID NO: 8866 represents the cDNA sequence for clone AI054143.

SEQ ID NO: 8867 represents the cDNA sequence for clone AI054145.

SEQ ID NO: 8868 represents the cDNA sequence for clone AI054148.

SEQ ID NO: 8869 represents the cDNA sequence for clone AI054149.

SEQ ID NO: 8870 represents the cDNA sequence for clone AI054151.

SEQ ID NO: 8871 represents the cDNA sequence for clone AI054153.

SEQ ID NO: 8872 represents the cDNA sequence for clone AI054159.

SEQ ID NO: 8873 represents the cDNA sequence for clone AI054174.

SEQ ID NO: 8874 represents the cDNA sequence for clone AI054180.

SEQ ID NO: 8875 represents the cDNA sequence for clone AI054182.

SEQ ID NO: 8876 represents the cDNA sequence for clone AI054184.

SEQ ID NO: 8877 represents the cDNA sequence for clone AI054192.

SEQ ID NO: 8878 represents the cDNA sequence for clone AI054195.

SEQ ID NO: 8879 represents the cDNA sequence for clone AI054198.

SEQ ID NO: 8880 represents the cDNA sequence for clone AI054200.

SEQ ID NO: 8881 represents the cDNA sequence for clone AI054208.

SEQ ID NO: 8882 represents the cDNA sequence for clone AI054213.

SEQ ID NO: 8883 represents the cDNA sequence for clone AI054216.

SEQ ID NO: 8884 represents the cDNA sequence for clone AI054219.

SEQ ID NO: 8885 represents the cDNA sequence for clone AI054220.

SEQ ID NO: 8886 represents the cDNA sequence for clone AI054221.

SEQ ID NO: 8887 represents the cDNA sequence for clone AI054222.

SEQ ID NO: 8888 represents the cDNA sequence for clone AI054223.

SEQ ID NO: 8889 represents the cDNA sequence for clone AI054225.

SEQ ID NO: 8890 represents the cDNA sequence for clone AI054226.

SEQ ID NO: 8891 represents the cDNA sequence for clone AI054229.

SEQ ID NO: 8892 represents the cDNA sequence for clone AI054231.

SEQ ID NO: 8893 represents the cDNA sequence for clone AI054232.

SEQ ID NO: 8894 represents the cDNA sequence for clone AI054234.

SEQ ID NO: 8895 represents the cDNA sequence for clone AI054235.

SEQ ID NO: 8896 represents the cDNA sequence for clone AI054244.

SEQ ID NO: 8897 represents the cDNA sequence for clone AI054246.

SEQ ID NO: 8898 represents the cDNA sequence for clone AI054251.

SEQ ID NO: 8899 represents the cDNA sequence for clone AI054252.

SEQ ID NO: 8900 represents the cDNA sequence for clone AI054256.

SEQ ID NO: 8901 represents the cDNA sequence for clone AI054258.

SEQ ID NO: 8902 represents the cDNA sequence for clone AI054260.

SEQ ID NO: 8903 represents the cDNA sequence for clone AI054269.

SEQ ID NO: 8904 represents the cDNA sequence for clone AI054272.

SEQ ID NO: 8905 represents the cDNA sequence for clone AI054275.

SEQ ID NO: 8906 represents the cDNA sequence for clone AI054276.

SEQ ID NO: 8907 represents the cDNA sequence for clone AI054280.

SEQ ID NO: 8908 represents the cDNA sequence for clone AI054299.

SEQ ID NO: 8909 represents the cDNA sequence for clone AI054306.

SEQ ID NO: 8910 represents the cDNA sequence for clone AI054310.

SEQ ID NO: 8911 represents the cDNA sequence for clone AI054314.

SEQ ID NO: 8912 represents the cDNA sequence for clone AI054317.

SEQ ID NO: 8913 represents the cDNA sequence for clone AI054320.

SEQ ID NO: 8914 represents the cDNA sequence for clone AI054323.

SEQ ID NO: 8915 represents the cDNA sequence for clone AI054325.

SEQ ID NO: 8916 represents the cDNA sequence for clone AI054326.

SEQ ID NO: 8917 represents the cDNA sequence for clone AI054327.

SEQ ID NO: 8918 represents the cDNA sequence for clone AI054328.

SEQ ID NO: 8919 represents the cDNA sequence for clone AI054331.

SEQ ID NO: 8920 represents the cDNA sequence for clone AI054333.

SEQ ID NO: 8921 represents the cDNA sequence for clone AI054340.

SEQ ID NO: 8922 represents the cDNA sequence for clone AI054343.

SEQ ID NO: 8923 represents the cDNA sequence for clone AI054345.

SEQ ID NO: 8924 represents the cDNA sequence for clone AI054351.

SEQ ID NO: 8925 represents the cDNA sequence for clone AI054352.

SEQ ID NO: 8926 represents the cDNA sequence for clone AI054353.

SEQ ID NO: 8927 represents the cDNA sequence for clone AI054354.

SEQ ID NO: 8928 represents the cDNA sequence for clone AI054360.

SEQ ID NO: 8929 represents the cDNA sequence for clone AI054361.

SEQ ID NO: 8930 represents the cDNA sequence for clone AI054366.

SEQ ID NO: 8931 represents the cDNA sequence for clone AI054367.

SEQ ID NO: 8932 represents the cDNA sequence for clone AI054372.

SEQ ID NO: 8933 represents the cDNA sequence for clone AI054375.

SEQ ID NO: 8934 represents the cDNA sequence for clone AI054378.

SEQ ID NO: 8935 represents the cDNA sequence for clone AI054381.

SEQ ID NO: 8936 represents the cDNA sequence for clone AI054383.

SEQ ID NO: 8937 represents the cDNA sequence for clone AI054384.

SEQ ID NO: 8938 represents the cDNA sequence for clone AI054387.

SEQ ID NO: 8939 represents the cDNA sequence for clone AI054391.

SEQ ID NO: 8940 represents the cDNA sequence for clone AI054393.

SEQ ID NO: 8941 represents the cDNA sequence for clone AI054397.

SEQ ID NO: 8942 represents the cDNA sequence for clone AI054398.

SEQ ID NO: 8943 represents the cDNA sequence for clone AI054399.

SEQ ID NO: 8944 represents the cDNA sequence for clone AI054402.

SEQ ID NO: 8945 represents the cDNA sequence for clone AI054405.

SEQ ID NO: 8946 represents the cDNA sequence for clone AI054406.

SEQ ID NO: 8947 represents the cDNA sequence for clone AI054408.

SEQ ID NO: 8948 represents the cDNA sequence for clone AI054409.

SEQ ID NO: 8949 represents the cDNA sequence for clone AI054411.

SEQ ID NO: 8950 represents the cDNA sequence for clone AI054416.

SEQ ID NO: 8951 represents the cDNA sequence for clone AI054419.

SEQ ID NO: 8952 represents the cDNA sequence for clone AI054422.

SEQ ID NO: 8953 represents the cDNA sequence for clone AI044023.

SEQ ID NO: 8954 represents the cDNA sequence for clone AI044024.

SEQ ID NO: 8955 represents the cDNA sequence for clone AI144025.

SEQ ID NO: 8956 represents the cDNA sequence for clone AI144026.

SEQ ID NO: 8957 represents the cDNA sequence for clone AI144029.

SEQ ID NO: 8958 represents the cDNA sequence for clone AI144032.

SEQ ID NO: 8959 represents the cDNA sequence for clone AI144034.

SEQ ID NO: 8960 represents the cDNA sequence for clone AI144036.

SEQ ID NO: 8961 represents the cDNA sequence for clone AI144039.

SEQ ID NO: 8962 represents the cDNA sequence for clone AI144041.

SEQ ID NO: 8963 represents the cDNA sequence for clone AI144042.

SEQ ID NO: 8964 represents the cDNA sequence for clone AI144047.

SEQ ID NO: 8965 represents the cDNA sequence for clone AI144056.

SEQ ID NO: 8966 represents the cDNA sequence for clone AI144058.

SEQ ID NO: 8967 represents the cDNA sequence for clone AI144059.

SEQ ID NO: 8968 represents the cDNA sequence for clone AI144063.

SEQ ID NO: 8969 represents the cDNA sequence for clone AI144066.

SEQ ID NO: 8970 represents the cDNA sequence for clone AI144067.

SEQ ID NO: 8971 represents the cDNA sequence for clone AI144068.

SEQ ID NO: 8972 represents the cDNA sequence for clone AI144070.

SEQ ID NO: 8973 represents the cDNA sequence for clone AI144071.

SEQ ID NO: 8974 represents the cDNA sequence for clone AI144072.

SEQ ID NO: 8975 represents the cDNA sequence for clone AI144076.

SEQ ID NO: 8976 represents the cDNA sequence for clone AI144077.

SEQ ID NO: 8977 represents the cDNA sequence for clone AI144078.

SEQ ID NO: 8978 represents the cDNA sequence for clone AI144083.

SEQ ID NO: 8979 represents the cDNA sequence for clone AI144097.

SEQ ID NO: 8980 represents the cDNA sequence for clone AI144101.

SEQ ID NO: 8981 represents the cDNA sequence for clone AI144102.

SEQ ID NO: 8982 represents the cDNA sequence for clone AI144104.

SEQ ID NO: 8983 represents the cDNA sequence for clone AI144106.

SEQ ID NO: 8984 represents the cDNA sequence for clone AI144108.

SEQ ID NO: 8985 represents the cDNA sequence for clone AI223469.

SEQ ID NO: 8986 represents the cDNA sequence for clone AI223470.

SEQ ID NO: 8987 represents the cDNA sequence for clone AI223475.

SEQ ID NO: 8988 represents the cDNA sequence for clone AI223489.

SEQ ID NO: 8989 represents the cDNA sequence for clone AI223493.

SEQ ID NO: 8990 represents the cDNA sequence for clone AI223503.

SEQ ID NO: 8991 represents the cDNA sequence for clone AI223506.

SEQ ID NO: 8992 represents the cDNA sequence for clone AI223508.

SEQ ID NO: 8993 represents the cDNA sequence for clone AI223511.

SEQ ID NO: 8994 represents the cDNA sequence for clone AI223515.

SEQ ID NO: 8995 represents the cDNA sequence for clone AI223518.

SEQ ID NO: 8996 represents the cDNA sequence for clone AI223543.

SEQ ID NO: 8997 represents the cDNA sequence for clone AI223548.

SEQ ID NO: 8998 represents the cDNA sequence for clone AI223555.

SEQ ID NO: 8999 represents the cDNA sequence for clone AI223558.

SEQ ID NO: 9000 represents the cDNA sequence for clone AI223576.

SEQ ID NO: 9001 represents the cDNA sequence for clone AI223586.

SEQ ID NO: 9002 represents the cDNA sequence for clone AI223590.

SEQ ID NO: 9003 represents the cDNA sequence for clone AI223597.

SEQ ID NO: 9004 represents the cDNA sequence for clone AI223600.

SEQ ID NO: 9005 represents the cDNA sequence for clone AI223609.

SEQ ID NO: 9006 represents the cDNA sequence for clone AI223615.

SEQ ID NO: 9007 represents the cDNA sequence for clone AI223629.

SEQ ID NO: 9008 represents the cDNA sequence for clone AI223642.

SEQ ID NO: 9009 represents the cDNA sequence for clone AI223653.

SEQ ID NO: 9010 represents the cDNA sequence for clone AI223666.

SEQ ID NO: 9011 represents the cDNA sequence for clone AI223676.

SEQ ID NO: 9012 represents the cDNA sequence for clone AI223677.

SEQ ID NO: 9013 represents the cDNA sequence for clone AI223678.

SEQ ID NO: 9014 represents the cDNA sequence for clone AI223698.

SEQ ID NO: 9015 represents the cDNA sequence for clone AI223700.

SEQ ID NO: 9016 represents the cDNA sequence for clone AI223706.

SEQ ID NO: 9017 represents the cDNA sequence for clone AI223739.

SEQ ID NO: 9018 represents the cDNA sequence for clone AI223752.

SEQ ID NO: 9019 represents the cDNA sequence for clone AI223756.

SEQ ID NO: 9020 represents the cDNA sequence for clone AI223757.

SEQ ID NO: 9021 represents the cDNA sequence for clone AI223766.

SEQ ID NO: 9022 represents the cDNA sequence for clone AI223784.

SEQ ID NO: 9023 represents the cDNA sequence for clone AI224180.

SEQ ID NO: 9024 represents the cDNA sequence for clone AI224187.

SEQ ID NO: 9025 represents the cDNA sequence for clone AI224205.

SEQ ID NO: 9026 represents the cDNA sequence for clone AI224227.

SEQ ID NO: 9027 represents the cDNA sequence for clone AI224232.

SEQ ID NO: 9028 represents the cDNA sequence for clone AI224233.

SEQ ID NO: 9029 represents the cDNA sequence for clone AI224248.

SEQ ID NO: 9030 represents the cDNA sequence for clone AI224270.

SEQ ID NO: 9031 represents the cDNA sequence for clone AI224711.

SEQ ID NO: 9032 represents the cDNA sequence for clone AI224718.

SEQ ID NO: 9033 represents the cDNA sequence for clone AI224735.

SEQ ID NO: 9034 represents the cDNA sequence for clone AI224736.

SEQ ID NO: 9035 represents the cDNA sequence for clone AI224738.

SEQ ID NO: 9036 represents the cDNA sequence for clone AI224751.

SEQ ID NO: 9037 represents the cDNA sequence for clone AI224757.

SEQ ID NO: 9038 represents the cDNA sequence for clone AI224760.

SEQ ID NO: 9039 represents the cDNA sequence for clone AI224765.

SEQ ID NO: 9040 represents the cDNA sequence for clone AI224777.

SEQ ID NO: 9041 represents the cDNA sequence for clone AI224800.

SEQ ID NO: 9042 represents the cDNA sequence for clone AI224819.

SEQ ID NO: 9043 represents the cDNA sequence for clone AI241970.

SEQ ID NO: 9044 represents the cDNA sequence for clone AI241981.

SEQ ID NO: 9045 represents the cDNA sequence for clone AI241985.

SEQ ID NO: 9046 represents the cDNA sequence for clone AI241987.

SEQ ID NO: 9047 represents the cDNA sequence for clone AI241990.

SEQ ID NO: 9048 represents the cDNA sequence for clone AI241992.

SEQ ID NO: 9049 represents the cDNA sequence for clone AI241993.

SEQ ID NO: 9050 represents the cDNA sequence for clone AI241998.

SEQ ID NO: 9051 represents the cDNA sequence for clone AI242000.

SEQ ID NO: 9052 represents the cDNA sequence for clone AI242006.

SEQ ID NO: 9053 represents the cDNA sequence for clone AI242014.

SEQ ID NO: 9054 represents the cDNA sequence for clone AI242019.

SEQ ID NO: 9055 represents the cDNA sequence for clone AI242020.

SEQ ID NO: 9056 represents the cDNA sequence for clone AI242227.

SEQ ID NO: 9057 represents the cDNA sequence for clone AI242236.

SEQ ID NO: 9058 represents the cDNA sequence for clone AI242240.

SEQ ID NO: 9059 represents the cDNA sequence for clone AI242241.

SEQ ID NO: 9060 represents the cDNA sequence for clone AI242250.

SEQ ID NO: 9061 represents the cDNA sequence for clone AI242255.

SEQ ID NO: 9062 represents the cDNA sequence for clone AI242256.

SEQ ID NO: 9063 represents the cDNA sequence for clone AI242261.

SEQ ID NO: 9064 represents the cDNA sequence for clone AI242263.

SEQ ID NO: 9065 represents the cDNA sequence for clone AI242268.

SEQ ID NO: 9066 represents the cDNA sequence for clone AI246059.

SEQ ID NO: 9067 represents the cDNA sequence for clone AI246069.

SEQ ID NO: 9068 represents the cDNA sequence for clone AI246088.

SEQ ID NO: 9069 represents the cDNA sequence for clone AI246796.

SEQ ID NO: 9070 represents the cDNA sequence for clone AI246809.

SEQ ID NO: 9071 represents the cDNA sequence for clone AI246816.

SEQ ID NO: 9072 represents the cDNA sequence for clone AI246819.

SEQ ID NO: 9073 represents the cDNA sequence for clone AI246821.

SEQ ID NO: 9074 represents the cDNA sequence for clone AI246833.

SEQ ID NO: 9075 represents the cDNA sequence for clone AI246856.

SEQ ID NO: 9076 represents the cDNA sequence for clone AI246860.

SEQ ID NO: 9077 represents the cDNA sequence for clone AI246864.

SEQ ID NO: 9078 represents the cDNA sequence for clone AI246928.

SEQ ID NO: 9079 represents the cDNA sequence for clone AI246934.

SEQ ID NO: 9080 represents the cDNA sequence for clone AI246962.

SEQ ID NO: 9081 represents the cDNA sequence for clone AI246971.

SEQ ID NO: 9082 represents the cDNA sequence for clone AI246986.

SEQ ID NO: 9083 represents the cDNA sequence for clone AI247034.

SEQ ID NO: 9084 represents the cDNA sequence for clone AI247066.

SEQ ID NO: 9085 represents the cDNA sequence for clone AI247150.

SEQ ID NO: 9086 represents the cDNA sequence for clone AI247160.

SEQ ID NO: 9087 represents the cDNA sequence for clone AI249277.

SEQ ID NO: 9088 represents the cDNA sequence for clone AI249279.

SEQ ID NO: 9089 represents the cDNA sequence for clone AI249285.

SEQ ID NO: 9090 represents the cDNA sequence for clone AI249312.

SEQ ID NO: 9091 represents the cDNA sequence for clone AI249317.

SEQ ID NO: 9092 represents the cDNA sequence for clone AI249402.

SEQ ID NO: 9093 represents the cDNA sequence for clone AI249412.

SEQ ID NO: 9094 represents the cDNA sequence for clone AI249415.

SEQ ID NO: 9095 represents the cDNA sequence for clone AI249422.

SEQ ID NO: 9096 represents the cDNA sequence for clone AI249531.

SEQ ID NO: 9097 represents the cDNA sequence for clone AI249535.

SEQ ID NO: 9098 represents the cDNA sequence for clone AI249542.

SEQ ID NO: 9099 represents the cDNA sequence for clone AI249544.

SEQ ID NO: 9100 represents the cDNA sequence for clone AI249547.

SEQ ID NO: 9101 represents the cDNA sequence for clone AI249556.

SEQ ID NO: 9102 represents the cDNA sequence for clone AI249562.

SEQ ID NO: 9103 represents the cDNA sequence for clone AI249572.

SEQ ID NO: 9104 represents the cDNA sequence for clone AI249576.

SEQ ID NO: 9105 represents the cDNA sequence for clone AI249638.

SEQ ID NO: 9106 represents the cDNA sequence for clone AI249647.

SEQ ID NO: 9107 represents the cDNA sequence for clone AI249654.

SEQ ID NO: 9108 represents the cDNA sequence for clone AI249835.

SEQ ID NO: 9109 represents the cDNA sequence for clone AI249843.

SEQ ID NO: 9110 represents the cDNA sequence for clone AI249848.

SEQ ID NO: 9111 represents the cDNA sequence for clone AI249849.

SEQ ID NO: 9112 represents the cDNA sequence for clone AI249867.

SEQ ID NO: 9113 represents the cDNA sequence for clone AI249993.

SEQ ID NO: 9114 represents the cDNA sequence for clone AI249998.

SEQ ID NO: 9115 represents the cDNA sequence for clone AI250002.

SEQ ID NO: 9116 represents the cDNA sequence for clone AI250006.

SEQ ID NO: 9117 represents the cDNA sequence for clone AI250020.

SEQ ID NO: 9118 represents the cDNA sequence for clone AI250027.

SEQ ID NO: 9119 represents the cDNA sequence for clone AI250030.

SEQ ID NO: 9120 represents the cDNA sequence for clone AI250081.

SEQ ID NO: 9121 represents the cDNA sequence for clone AI250101.

SEQ ID NO: 9122 represents the cDNA sequence for clone AI250122.

SEQ ID NO: 9123 represents the cDNA sequence for clone AI250139.

SEQ ID NO: 9124 represents the cDNA sequence for clone AI250222.

SEQ ID NO: 9125 represents the cDNA sequence for clone AI250225.

SEQ ID NO: 9126 represents the cDNA sequence for clone AI250375.

SEQ ID NO: 9127 represents the cDNA sequence for clone AI250376.

SEQ ID NO: 9128 represents the cDNA sequence for clone AI250428.

SEQ ID NO: 9129 represents the cDNA sequence for clone AI250451.

SEQ ID NO: 9130 represents the cDNA sequence for clone AI250463.

SEQ ID NO: 9131 represents the cDNA sequence for clone AI250509.

SEQ ID NO: 9132 represents the cDNA sequence for clone AI250515.

SEQ ID NO: 9133 represents the cDNA sequence for clone AI250518.

SEQ ID NO: 9134 represents the cDNA sequence for clone AI250523.

SEQ ID NO: 9135 represents the cDNA sequence for clone AI250527.

SEQ ID NO: 9136 represents the cDNA sequence for clone AI250539.

SEQ ID NO: 9137 represents the cDNA sequence for clone AI250548.

SEQ ID NO: 9138 represents the cDNA sequence for clone AI250559.

SEQ ID NO: 9139 represents the cDNA sequence for clone AI250569.

SEQ ID NO: 9140 represents the cDNA sequence for clone AI250584.

SEQ ID NO: 9141 represents the cDNA sequence for clone AI250589.

SEQ ID NO: 9142 represents the cDNA sequence for clone AI250594.

SEQ ID NO: 9143 represents the cDNA sequence for clone AI250597.

SEQ ID NO: 9144 represents the cDNA sequence for clone AI250710.

SEQ ID NO: 9145 represents the cDNA sequence for clone AI250712.

SEQ ID NO: 9146 represents the cDNA sequence for clone AI250718.

SEQ ID NO: 9147 represents the cDNA sequence for clone AI250742.

SEQ ID NO: 9148 represents the cDNA sequence for clone AI250953.

SEQ ID NO: 9149 represents the cDNA sequence for clone AI250965.

SEQ ID NO: 9150 represents the cDNA sequence for clone AI250966.

SEQ ID NO: 9151 represents the cDNA sequence for clone AI250974.

SEQ ID NO: 9152 represents the cDNA sequence for clone AI250984.

SEQ ID NO: 9153 represents the cDNA sequence for clone AI251037.

SEQ ID NO: 9154 represents the cDNA sequence for clone AI251040.

SEQ ID NO: 9155 represents the cDNA sequence for clone AI251066.

SEQ ID NO: 9156 represents the cDNA sequence for clone AI251068.

SEQ ID NO: 9157 represents the cDNA sequence for clone AI251083.

SEQ ID NO: 9158 represents the cDNA sequence for clone AI251089.

SEQ ID NO: 9159 represents the cDNA sequence for clone AI251135.

SEQ ID NO: 9160 represents the cDNA sequence for clone AI251139.

SEQ ID NO: 9161 represents the cDNA sequence for clone AI251140.

SEQ ID NO: 9162 represents the cDNA sequence for clone AI251145.

SEQ ID NO: 9163 represents the cDNA sequence for clone AI251148.

SEQ ID NO: 9164 represents the cDNA sequence for clone AI251149.

SEQ ID NO: 9165 represents the cDNA sequence for clone AI251151.

SEQ ID NO: 9166 represents the cDNA sequence for clone AI251157.

SEQ ID NO: 9167 represents the cDNA sequence for clone AI251159.

SEQ ID NO: 9168 represents the cDNA sequence for clone AI251164.

SEQ ID NO: 9169 represents the cDNA sequence for clone AI251165.

SEQ ID NO: 9170 represents the cDNA sequence for clone AI251167.

SEQ ID NO: 9171 represents the cDNA sequence for clone AI251170.

SEQ ID NO: 9172 represents the cDNA sequence for clone AI251179.

SEQ ID NO: 9173 represents the cDNA sequence for clone AI251182.

SEQ ID NO: 9174 represents the cDNA sequence for clone AI251183.

SEQ ID NO: 9175 represents the cDNA sequence for clone AI251185.

SEQ ID NO: 9176 represents the cDNA sequence for clone AI251192.

SEQ ID NO: 9177 represents the cDNA sequence for clone AI251207.

SEQ ID NO: 9178 represents the cDNA sequence for clone AI251214.

SEQ ID NO: 9179 represents the cDNA sequence for clone AI251217.

SEQ ID NO: 9180 represents the cDNA sequence for clone AI251219.

SEQ ID NO: 9181 represents the cDNA sequence for clone AI251223.

SEQ ID NO: 9182 represents the cDNA sequence for clone AI251225.

SEQ ID NO: 9183 represents the cDNA sequence for clone AI251234.

SEQ ID NO: 9184 represents the cDNA sequence for clone AI251235.

SEQ ID NO: 9185 represents the cDNA sequence for clone AI251238.

SEQ ID NO: 9186 represents the cDNA sequence for clone AI251241.

SEQ ID NO: 9187 represents the cDNA sequence for clone AI251253.

SEQ ID NO: 9188 represents the cDNA sequence for clone AI251255.

SEQ ID NO: 9189 represents the cDNA sequence for clone AI251259.

SEQ ID NO: 9190 represents the cDNA sequence for clone AI251276.

SEQ ID NO: 9191 represents the cDNA sequence for clone AI251277.

SEQ ID NO: 9192 represents the cDNA sequence for clone AI251283.

SEQ ID NO: 9193 represents the cDNA sequence for clone AI251291.

SEQ ID NO: 9194 represents the cDNA sequence for clone AI251294.

SEQ ID NO: 9195 represents the cDNA sequence for clone AI251298.

SEQ ID NO: 9196 represents the cDNA sequence for clone AI251299.

SEQ ID NO: 9197 represents the cDNA sequence for clone AI251305.

SEQ ID NO: 9198 represents the cDNA sequence for clone AI251307.

SEQ ID NO: 9199 represents the cDNA sequence for clone AI251310.

SEQ ID NO: 9200 represents the cDNA sequence for clone AI251317.

SEQ ID NO: 9201 represents the cDNA sequence for clone AI251321.

SEQ ID NO: 9202 represents the cDNA sequence for clone AI251322.

SEQ ID NO: 9203 represents the cDNA sequence for clone AI251329.

SEQ ID NO: 9204 represents the cDNA sequence for clone AI251331.

SEQ ID NO: 9205 represents the cDNA sequence for clone AI251336.

SEQ ID NO: 9206 represents the cDNA sequence for clone AI251339.

SEQ ID NO: 9207 represents the cDNA sequence for clone AI251343.

SEQ ID NO: 9208 represents the cDNA sequence for clone AI251353.

SEQ ID NO: 9209 represents the cDNA sequence for clone AI251355.

SEQ ID NO: 9210 represents the cDNA sequence for clone AI251359.

SEQ ID NO: 9211 represents the cDNA sequence for clone AI251369.

SEQ ID NO: 9212 represents the cDNA sequence for clone AI251370.

SEQ ID NO: 9213 represents the cDNA sequence for clone AI251376.

SEQ ID NO: 9214 represents the cDNA sequence for clone AI251380.

SEQ ID NO: 9215 represents the cDNA sequence for clone AI251381.

SEQ ID NO: 9216 represents the cDNA sequence for clone AI251385.

SEQ ID NO: 9217 represents the cDNA sequence for clone AI251386.

SEQ ID NO: 9218 represents the cDNA sequence for clone AI251390.

SEQ ID NO: 9219 represents the cDNA sequence for clone AI251395.

SEQ ID NO: 9220 represents the cDNA sequence for clone AI251398.

SEQ ID NO: 9221 represents the cDNA sequence for clone AI251399.

SEQ ID NO: 9222 represents the cDNA sequence for clone AI251403.

SEQ ID NO: 9223 represents the cDNA sequence for clone AI251424.

SEQ ID NO: 9224 represents the cDNA sequence for clone AI251426.

SEQ ID NO: 9225 represents the cDNA sequence for clone AI251495.

SEQ ID NO: 9226 represents the cDNA sequence for clone AI251497.

SEQ ID NO: 9227 represents the cDNA sequence for clone AI251499.

SEQ ID NO: 9228 represents the cDNA sequence for clone AI251518.

SEQ ID NO: 9229 represents the cDNA sequence for clone AI251536.

SEQ ID NO: 9230 represents the cDNA sequence for clone AI251537.

SEQ ID NO: 9231 represents the cDNA sequence for clone AI251542.

SEQ ID NO: 9232 represents the cDNA sequence for clone AI251549.

SEQ ID NO: 9233 represents the cDNA sequence for clone AI251552.

SEQ ID NO: 9234 represents the cDNA sequence for clone AI251553.

SEQ ID NO: 9235 represents the cDNA sequence for clone AI251559.

SEQ ID NO: 9236 represents the cDNA sequence for clone AI251563.

SEQ ID NO: 9237 represents the cDNA sequence for clone AI251568.

SEQ ID NO: 9238 represents the cDNA sequence for clone AI251570.

SEQ ID NO: 9239 represents the cDNA sequence for clone AI251572.

SEQ ID NO: 9240 represents the cDNA sequence for clone AI251574.

SEQ ID NO: 9241 represents the cDNA sequence for clone AI251581.

SEQ ID NO: 9242 represents the cDNA sequence for clone AI251583.

SEQ ID NO: 9243 represents the cDNA sequence for clone AI251589.

SEQ ID NO: 9244 represents the cDNA sequence for clone AI251590.

SEQ ID NO: 9245 represents the cDNA sequence for clone AI251594.

SEQ ID NO: 9246 represents the cDNA sequence for clone AI251596.

SEQ ID NO: 9247 represents the cDNA sequence for clone AI251598.

SEQ ID NO: 9248 represents the cDNA sequence for clone AI251611.

SEQ ID NO: 9249 represents the cDNA sequence for clone AI251613.

SEQ ID NO: 9250 represents the cDNA sequence for clone AI251615.

SEQ ID NO: 9251 represents the cDNA sequence for clone AI251622.

SEQ ID NO: 9252 represents the cDNA sequence for clone AI251623.

SEQ ID NO: 9253 represents the cDNA sequence for clone AI251636.

SEQ ID NO: 9254 represents the cDNA sequence for clone AI251638.

SEQ ID NO: 9255 represents the cDNA sequence for clone AI251639.

SEQ ID NO: 9256 represents the cDNA sequence for clone AI251640.

SEQ ID NO: 9257 represents the cDNA sequence for clone AI251642.

SEQ ID NO: 9258 represents the cDNA sequence for clone AI251649.

SEQ ID NO: 9259 represents the cDNA sequence for clone AI251672.

SEQ ID NO: 9260 represents the cDNA sequence for clone AI251681.

SEQ ID NO: 9261 represents the cDNA sequence for clone AI251686.

SEQ ID NO: 9262 represents the cDNA sequence for clone AI251690.

SEQ ID NO: 9263 represents the cDNA sequence for clone AI251692.

SEQ ID NO: 9264 represents the cDNA sequence for clone AI251700.

SEQ ID NO: 9265 represents the cDNA sequence for clone AI251703.

SEQ ID NO: 9266 represents the cDNA sequence for clone AI251705.

SEQ ID NO: 9267 represents the cDNA sequence for clone AI251710.

SEQ ID NO: 9268 represents the cDNA sequence for clone AI251711.

SEQ ID NO: 9269 represents the cDNA sequence for clone AI251712.

SEQ ID NO: 9270 represents the cDNA sequence for clone AI251715.

SEQ ID NO: 9271 represents the cDNA sequence for clone AI251722.

SEQ ID NO: 9272 represents the cDNA sequence for clone AI251724.

SEQ ID NO: 9273 represents the cDNA sequence for clone AI251726.

SEQ ID NO: 9274 represents the cDNA sequence for clone AI251944.

SEQ ID NO: 9275 represents the cDNA sequence for clone AI251948.

SEQ ID NO: 9276 represents the cDNA sequence for clone AI251949.

SEQ ID NO: 9277 represents the cDNA sequence for clone AI251959.

SEQ ID NO: 9278 represents the cDNA sequence for clone AI251966.

SEQ ID NO: 9279 represents the cDNA sequence for clone AI251968.

SEQ ID NO: 9280 represents the cDNA sequence for clone AI251969.

SEQ ID NO: 9281 represents the cDNA sequence for clone AI251971.

SEQ ID NO: 9282 represents the cDNA sequence for clone AI251976.

SEQ ID NO: 9283 represents the cDNA sequence for clone AI251978.

SEQ ID NO: 9284 represents the cDNA sequence for clone AI251979.

SEQ ID NO: 9285 represents the cDNA sequence for clone AI251980.

SEQ ID NO: 9286 represents the cDNA sequence for clone AI251981.

SEQ ID NO: 9287 represents the cDNA sequence for clone AI251983.

SEQ ID NO: 9288 represents the cDNA sequence for clone AI251987.

SEQ ID NO: 9289 represents the cDNA sequence for clone AI251988.

SEQ ID NO: 9290 represents the cDNA sequence for clone AI251990.

SEQ ID NO: 9291 represents the cDNA sequence for clone AI251991.

SEQ ID NO: 9292 represents the cDNA sequence for clone AI251992.

SEQ ID NO: 9293 represents the cDNA sequence for clone AI251996.

SEQ ID NO: 9294 represents the cDNA sequence for clone AI251999.

SEQ ID NO: 9295 represents the cDNA sequence for clone AI252004.

SEQ ID NO: 9296 represents the cDNA sequence for clone AI252009.

SEQ ID NO: 9297 represents the cDNA sequence for clone AI252010.

SEQ ID NO: 9298 represents the cDNA sequence for clone AI252013.

SEQ ID NO: 9299 represents the cDNA sequence for clone AI252014.

SEQ ID NO: 9300 represents the cDNA sequence for clone AI252015.

SEQ ID NO: 9301 represents the cDNA sequence for clone AI252025.

SEQ ID NO: 9302 represents the cDNA sequence for clone AI252035.

SEQ ID NO: 9303 represents the cDNA sequence for clone AI252053.

SEQ ID NO: 9304 represents the cDNA sequence for clone AI252054.

SEQ ID NO: 9305 represents the cDNA sequence for clone AI252062.

SEQ ID NO: 9306 represents the cDNA sequence for clone AI252065.

SEQ ID NO: 9307 represents the cDNA sequence for clone AI252070.

SEQ ID NO: 9308 represents the cDNA sequence for clone AI252072.

SEQ ID NO: 9309 represents the cDNA sequence for clone AI252073.

SEQ ID NO: 9310 represents the cDNA sequence for clone AI252076.

SEQ ID NO: 9311 represents the cDNA sequence for clone AI252079.

SEQ ID NO: 9312 represents the cDNA sequence for clone AI252080.

SEQ ID NO: 9313 represents the cDNA sequence for clone AI252081.

SEQ ID NO: 9314 represents the cDNA sequence for clone AI252085.

SEQ ID NO: 9315 represents the cDNA sequence for clone AI252086.

SEQ ID NO: 9316 represents the cDNA sequence for clone AI252087.

SEQ ID NO: 9317 represents the cDNA sequence for clone AI252092.

SEQ ID NO: 9318 represents the cDNA sequence for clone AI252093.

SEQ ID NO: 9319 represents the cDNA sequence for clone AI252098.

SEQ ID NO: 9320 represents the cDNA sequence for clone AI252105.

SEQ ID NO: 9321 represents the cDNA sequence for clone AI252107.

SEQ ID NO: 9322 represents the cDNA sequence for clone AI252109.

SEQ ID NO: 9323 represents the cDNA sequence for clone AI252111.

SEQ ID NO: 9324 represents the cDNA sequence for clone AI252112.

SEQ ID NO: 9325 represents the cDNA sequence for clone AI252113.

SEQ ID NO: 9326 represents the cDNA sequence for clone AI252114.

SEQ ID NO: 9327 represents the cDNA sequence for clone AI252122.

SEQ ID NO: 9328 represents the cDNA sequence for clone AI252125.

SEQ ID NO: 9329 represents the cDNA sequence for clone AI252129.

SEQ ID NO: 9330 represents the cDNA sequence for clone AI252130.

SEQ ID NO: 9331 represents the cDNA sequence for clone AI252132.

SEQ ID NO: 9332 represents the cDNA sequence for clone AI252133.

SEQ ID NO: 9333 represents the cDNA sequence for clone AI252136.

SEQ ID NO: 9334 represents the cDNA sequence for clone AI252139.

SEQ ID NO: 9335 represents the cDNA sequence for clone AI252141.

SEQ ID NO: 9336 represents the cDNA sequence for clone AI252154.

SEQ ID NO: 9337 represents the cDNA sequence for clone AI252157.

SEQ ID NO: 9338 represents the cDNA sequence for clone AI252171.

SEQ ID NO: 9339 represents the cDNA sequence for clone AI252175.

SEQ ID NO: 9340 represents the cDNA sequence for clone AI252180.

SEQ ID NO: 9341 represents the cDNA sequence for clone AI252186.

SEQ ID NO: 9342 represents the cDNA sequence for clone AI252188.

SEQ ID NO: 9343 represents the cDNA sequence for clone AI252190.

SEQ ID NO: 9344 represents the cDNA sequence for clone AI252191.

SEQ ID NO: 9345 represents the cDNA sequence for clone AI252192.

SEQ ID NO: 9346 represents the cDNA sequence for clone AI252197.

SEQ ID NO: 9347 represents the cDNA sequence for clone AI252199.

SEQ ID NO: 9348 represents the cDNA sequence for clone AI252200.

SEQ ID NO: 9349 represents the cDNA sequence for clone AI252207.

SEQ ID NO: 9350 represents the cDNA sequence for clone AI252245.

SEQ ID NO: 9351 represents the cDNA sequence for clone AI252272.

SEQ ID NO: 9352 represents the cDNA sequence for clone AI252273.

SEQ ID NO: 9353 represents the cDNA sequence for clone AI252285.

SEQ ID NO: 9354 represents the cDNA sequence for clone AI252287.

SEQ ID NO: 9355 represents the cDNA sequence for clone AI252294.

SEQ ID NO: 9356 represents the cDNA sequence for clone AI252295.

SEQ ID NO: 9357 represents the cDNA sequence for clone AI252309.

SEQ ID NO: 9358 represents the cDNA sequence for clone AI252315.

SEQ ID NO: 9359 represents the cDNA sequence for clone AI252324.

SEQ ID NO: 9360 represents the cDNA sequence for clone AI252336.

SEQ ID NO: 9361 represents the cDNA sequence for clone AI252339.

SEQ ID NO: 9362 represents the cDNA sequence for clone AI252343.

SEQ ID NO: 9363 represents the cDNA sequence for clone AI252382.

SEQ ID NO: 9364 represents the cDNA sequence for clone AI252383.

SEQ ID NO: 9365 represents the cDNA sequence for clone AI252399.

SEQ ID NO: 9366 represents the cDNA sequence for clone AI252419.

SEQ ID NO: 9367 represents the cDNA sequence for clone AI252422.

SEQ ID NO: 9368 represents the cDNA sequence for clone AI252429.

SEQ ID NO: 9369 represents the cDNA sequence for clone AI252430.

SEQ ID NO: 9370 represents the cDNA sequence for clone AI252431.

SEQ ID NO: 9371 represents the cDNA sequence for clone AI252438.

SEQ ID NO: 9372 represents the cDNA sequence for clone AI252442.

SEQ ID NO: 9373 represents the cDNA sequence for clone AI252456.

SEQ ID NO: 9374 represents the cDNA sequence for clone AI252458.

SEQ ID NO: 9375 represents the cDNA sequence for clone AI252466.

SEQ ID NO: 9376 represents the cDNA sequence for clone AI252476.

SEQ ID NO: 9377 represents the cDNA sequence for clone AI252490.

SEQ ID NO: 9378 represents the cDNA sequence for clone AI252492.

SEQ ID NO: 9379 represents the cDNA sequence for clone AI252496.

SEQ ID NO: 9380 represents the cDNA sequence for clone AI252497.

SEQ ID NO: 9381 represents the cDNA sequence for clone AI252504.

SEQ ID NO: 9382 represents the cDNA sequence for clone AI252526.

SEQ ID NO: 9383 represents the cDNA sequence for clone AI252530.

SEQ ID NO: 9384 represents the cDNA sequence for clone AI252547.

SEQ ID NO: 9385 represents the cDNA sequence for clone AI252549.

SEQ ID NO: 9386 represents the cDNA sequence for clone AI252551.

SEQ ID NO: 9387 represents the cDNA sequence for clone AI252554.

SEQ ID NO: 9388 represents the cDNA sequence for clone AI252573.

SEQ ID NO: 9389 represents the cDNA sequence for clone AI252580.

SEQ ID NO: 9390 represents the cDNA sequence for clone AI252584.

SEQ ID NO: 9391 represents the cDNA sequence for clone AI252586.

SEQ ID NO: 9392 represents the cDNA sequence for clone AI252594.

SEQ ID NO: 9393 represents the cDNA sequence for clone AI252609.

SEQ ID NO: 9394 represents the cDNA sequence for clone AI252688.

SEQ ID NO: 9395 represents the cDNA sequence for clone AI252695.

SEQ ID NO: 9396 represents the cDNA sequence for clone AI252700.

SEQ ID NO: 9397 represents the cDNA sequence for clone AI252701.

SEQ ID NO: 9398 represents the cDNA sequence for clone AI252710.

SEQ ID NO: 9399 represents the cDNA sequence for clone AI252712.

SEQ ID NO: 9400 represents the cDNA sequence for clone AI252721.

SEQ ID NO: 9401 represents the cDNA sequence for clone AI252724.

SEQ ID NO: 9402 represents the cDNA sequence for clone AI252738.

SEQ ID NO: 9403 represents the cDNA sequence for clone AI252746.

SEQ ID NO: 9404 represents the cDNA sequence for clone AI252748.

SEQ ID NO: 9405 represents the cDNA sequence for clone AI252823.

SEQ ID NO: 9406 represents the cDNA sequence for clone AI252827.

SEQ ID NO: 9407 represents the cDNA sequence for clone AI252828.

SEQ ID NO: 9408 represents the cDNA sequence for clone AI252832.

SEQ ID NO: 9409 represents the cDNA sequence for clone AI252836.

SEQ ID NO: 9410 represents the cDNA sequence for clone AI252838.

SEQ ID NO: 9411 represents the cDNA sequence for clone AI252843.

SEQ ID NO: 9412 represents the cDNA sequence for clone AI252845.

SEQ ID NO: 9413 represents the cDNA sequence for clone AI252849.

SEQ ID NO: 9414 represents the cDNA sequence for clone AI252851.

SEQ ID NO: 9415 represents the cDNA sequence for clone AI252857.

SEQ ID NO: 9416 represents the cDNA sequence for clone AI252858.

SEQ ID NO: 9417 represents the cDNA sequence for clone AI252861.

SEQ ID NO: 9418 represents the cDNA sequence for clone AI252863.

SEQ ID NO: 9419 represents the cDNA sequence for clone AI252869.

SEQ ID NO: 9420 represents the cDNA sequence for clone AI252871.

SEQ ID NO: 9421 represents the cDNA sequence for clone AI252878.

SEQ ID NO: 9422 represents the cDNA sequence for clone AI252880.

SEQ ID NO: 9423 represents the cDNA sequence for clone AI252886.

SEQ ID NO: 9424 represents the cDNA sequence for clone AI252890.

SEQ ID NO: 9425 represents the cDNA sequence for clone AI252892.

SEQ ID NO: 9426 represents the cDNA sequence for clone AI252899.

SEQ ID NO: 9427 represents the cDNA sequence for clone AI252903.

SEQ ID NO: 9428 represents the cDNA sequence for clone AI252905.

SEQ ID NO: 9429 represents the cDNA sequence for clone AI252911.

SEQ ID NO: 9430 represents the cDNA sequence for clone AI252915.

SEQ ID NO: 9431 represents the cDNA sequence for clone AI252918.

SEQ ID NO: 9432 represents the cDNA sequence for clone AI252922.

SEQ ID NO: 9433 represents the cDNA sequence for clone AI252924.

SEQ ID NO: 9434 represents the cDNA sequence for clone AI252925.

SEQ ID NO: 9435 represents the cDNA sequence for clone AI252933.

SEQ ID NO: 9436 represents the cDNA sequence for clone AI252934.

SEQ ID NO: 9437 represents the cDNA sequence for clone AI252935.

SEQ ID NO: 9438 represents the cDNA sequence for clone AI252938.

SEQ ID NO: 9439 represents the cDNA sequence for clone AI252940.

SEQ ID NO: 9440 represents the cDNA sequence for clone AI252942.

SEQ ID NO: 9441 represents the cDNA sequence for clone AI252943.

SEQ ID NO: 9442 represents the cDNA sequence for clone AI252945.

SEQ ID NO: 9443 represents the cDNA sequence for clone AI252958.

SEQ ID NO: 9444 represents the cDNA sequence for clone AI252960.

SEQ ID NO: 9445 represents the cDNA sequence for clone AI252963.

SEQ ID NO: 9446 represents the cDNA sequence for clone AI252967.

SEQ ID NO: 9447 represents the cDNA sequence for clone AI252969.

SEQ ID NO: 9448 represents the cDNA sequence for clone AI252971.

SEQ ID NO: 9449 represents the cDNA sequence for clone AI252976.

SEQ ID NO: 9450 represents the cDNA sequence for clone AI252978.

SEQ ID NO: 9451 represents the cDNA sequence for clone AI252979.

SEQ ID NO: 9452 represents the cDNA sequence for clone AI252985.

SEQ ID NO: 9453 represents the cDNA sequence for clone AI252987.

SEQ ID NO: 9454 represents the cDNA sequence for clone AI253000.

SEQ ID NO: 9455 represents the cDNA sequence for clone AI253007.

SEQ ID NO: 9456 represents the cDNA sequence for clone AI253010.

SEQ ID NO: 9457 represents the cDNA sequence for clone AI253970.

SEQ ID NO: 9458 represents the cDNA sequence for clone AI253971.

SEQ ID NO: 9459 represents the cDNA sequence for clone AI253983.

SEQ ID NO: 9460 represents the cDNA sequence for clone AI253986.

SEQ ID NO: 9461 represents the cDNA sequence for clone AI253989.

SEQ ID NO: 9462 represents the cDNA sequence for clone AI253990.

SEQ ID NO: 9463 represents the cDNA sequence for clone AI254008.

SEQ ID NO: 9464 represents the cDNA sequence for clone AI254013.

SEQ ID NO: 9465 represents the cDNA sequence for clone AI254018.

SEQ ID NO: 9466 represents the cDNA sequence for clone AI254027.

SEQ ID NO: 9467 represents the cDNA sequence for clone AI254029.

SEQ ID NO: 9468 represents the cDNA sequence for clone AI254031.

SEQ ID NO: 9469 represents the cDNA sequence for clone AI254039.

SEQ ID NO: 9470 represents the cDNA sequence for clone AI254051.

SEQ ID NO: 9471 represents the cDNA sequence for clone AI254054.

SEQ ID NO: 9472 represents the cDNA sequence for clone AI254063.

SEQ ID NO: 9473 represents the cDNA sequence for clone AI254071.

SEQ ID NO: 9474 represents the cDNA sequence for clone AI254077.

SEQ ID NO: 9475 represents the cDNA sequence for clone AI254078.

SEQ ID NO: 9476 represents the cDNA sequence for clone AI254079.

SEQ ID NO: 9477 represents the cDNA sequence for clone AI254083.

SEQ ID NO: 9478 represents the cDNA sequence for clone AI254085.

SEQ ID NO: 9479 represents the cDNA sequence for clone AI254088.

SEQ ID NO: 9480 represents the cDNA sequence for clone AI254095.

SEQ ID NO: 9481 represents the cDNA sequence for clone AI254098.

SEQ ID NO: 9482 represents the cDNA sequence for clone AI254099.

SEQ ID NO: 9483 represents the cDNA sequence for clone AI254102.

SEQ ID NO: 9484 represents the cDNA sequence for clone AI254104.

SEQ ID NO: 9485 represents the cDNA sequence for clone AI254106.

SEQ ID NO: 9486 represents the cDNA sequence for clone AI254129.

SEQ ID NO: 9487 represents the cDNA sequence for clone AI254131.

SEQ ID NO: 9488 represents the cDNA sequence for clone AI254136.

SEQ ID NO: 9489 represents the cDNA sequence for clone AI254137.

SEQ ID NO: 9490 represents the cDNA sequence for clone AI254141.

SEQ ID NO: 9491 represents the cDNA sequence for clone AI254142.

SEQ ID NO: 9492 represents the cDNA sequence for clone AI254145.

SEQ ID NO: 9493 represents the cDNA sequence for clone AI254146.

SEQ ID NO: 9494 represents the cDNA sequence for clone AI254147.

SEQ ID NO: 9495 represents the cDNA sequence for clone AI254149.

SEQ ID NO: 9496 represents the cDNA sequence for clone AI254150.

SEQ ID NO: 9497 represents the cDNA sequence for clone AI254151.

SEQ ID NO: 9498 represents the cDNA sequence for clone AI254153.

SEQ ID NO: 9499 represents the cDNA sequence for clone AI254158.

SEQ ID NO: 9500 represents the cDNA sequence for clone AI254164.

SEQ ID NO: 9501 represents the cDNA sequence for clone AI254165.

SEQ ID NO: 9502 represents the cDNA sequence for clone AI254168.

SEQ ID NO: 9503 represents the cDNA sequence for clone AI254173.

SEQ ID NO: 9504 represents the cDNA sequence for clone AI254175.

SEQ ID NO: 9505 represents the cDNA sequence for clone AI254176.

SEQ ID NO: 9506 represents the cDNA sequence for clone AI254180.

SEQ ID NO: 9507 represents the cDNA sequence for clone AI254183.

SEQ ID NO: 9508 represents the cDNA sequence for clone AI254188.

SEQ ID NO: 9509 represents the cDNA sequence for clone AI254190.

SEQ ID NO: 9510 represents the cDNA sequence for clone AI254194.

SEQ ID NO: 9511 represents the cDNA sequence for clone AI254253.

SEQ ID NO: 9512 represents the cDNA sequence for clone AI254255.

SEQ ID NO: 9513 represents the cDNA sequence for clone AI254266.

SEQ ID NO: 9514 represents the cDNA sequence for clone AI254278.

SEQ ID NO: 9515 represents the cDNA sequence for clone AI254279.

SEQ ID NO: 9516 represents the cDNA sequence for clone AI254298.

SEQ ID NO: 9517 represents the cDNA sequence for clone AI254302.

SEQ ID NO: 9518 represents the cDNA sequence for clone AI254303.

SEQ ID NO: 9519 represents the cDNA sequence for clone AI254305.

SEQ ID NO: 9520 represents the cDNA sequence for clone AI254306.

SEQ ID NO: 9521 represents the cDNA sequence for clone AI254310.

SEQ ID NO: 9522 represents the cDNA sequence for clone AI254313.

SEQ ID NO: 9523 represents the cDNA sequence for clone AI254317.

SEQ ID NO: 9524 represents the cDNA sequence for clone AI254318.

SEQ ID NO: 9525 represents the cDNA sequence for clone AI254325.

SEQ ID NO: 9526 represents the cDNA sequence for clone AI254331.

SEQ ID NO: 9527 represents the cDNA sequence for clone AI254368.

SEQ ID NO: 9528 represents the cDNA sequence for clone AI254379.

SEQ ID NO: 9529 represents the cDNA sequence for clone AI254396.

SEQ ID NO: 9530 represents the cDNA sequence for clone AI254397.

SEQ ID NO: 9531 represents the cDNA sequence for clone AI254406.

SEQ ID NO: 9532 represents the cDNA sequence for clone AI254420.

SEQ ID NO: 9533 represents the cDNA sequence for clone AI254440.

SEQ ID NO: 9534 represents the cDNA sequence for clone AI254446.

SEQ ID NO: 9535 represents the cDNA sequence for clone AI254450.

SEQ ID NO: 9536 represents the cDNA sequence for clone AI254451.

SEQ ID NO: 9537 represents the cDNA sequence for clone AI254452.

SEQ ID NO: 9538 represents the cDNA sequence for clone AI254455.

SEQ ID NO: 9539 represents the cDNA sequence for clone AI254460.

SEQ ID NO: 9540 represents the cDNA sequence for clone AI254462.

SEQ ID NO: 9541 represents the cDNA sequence for clone AI254471.

SEQ ID NO: 9542 represents the cDNA sequence for clone AI254472.

SEQ ID NO: 9543 represents the cDNA sequence for clone AI254473.

SEQ ID NO: 9544 represents the cDNA sequence for clone AI254474.

SEQ ID NO: 9545 represents the cDNA sequence for clone AI254480.

SEQ ID NO: 9546 represents the cDNA sequence for clone AI254485.

SEQ ID NO: 9547 represents the cDNA sequence for clone AI254490.

SEQ ID NO: 9548 represents the cDNA sequence for clone AI254493.

SEQ ID NO: 9549 represents the cDNA sequence for clone AI254507.

SEQ ID NO: 9550 represents the cDNA sequence for clone AI254509.

SEQ ID NO: 9551 represents the cDNA sequence for clone AI254510.

SEQ ID NO: 9552 represents the cDNA sequence for clone AI254511.

SEQ ID NO: 9553 represents the cDNA sequence for clone AI254513.

SEQ ID NO: 9554 represents the cDNA sequence for clone AI254514.

SEQ ID NO: 9555 represents the cDNA sequence for clone AI254520.

SEQ ID NO: 9556 represents the cDNA sequence for clone AI254521.

SEQ ID NO: 9557 represents the cDNA sequence for clone AI254524.

SEQ ID NO: 9558 represents the cDNA sequence for clone AI254526.

SEQ ID NO: 9559 represents the cDNA sequence for clone AI254527.

SEQ ID NO: 9560 represents the cDNA sequence for clone AI254528.

SEQ ID NO: 9561 represents the cDNA sequence for clone AI254529.

SEQ ID NO: 9562 represents the cDNA sequence for clone AI254532.

SEQ ID NO: 9563 represents the cDNA sequence for clone AI254543.

SEQ ID NO: 9564 represents the cDNA sequence for clone AI254547.

SEQ ID NO: 9565 represents the cDNA sequence for clone AI254550.

SEQ ID NO: 9566 represents the cDNA sequence for clone AI254553.

SEQ ID NO: 9567 represents the cDNA sequence for clone AI254561.

SEQ ID NO: 9568 represents the cDNA sequence for clone AI254565.

SEQ ID NO: 9569 represents the cDNA sequence for clone AI254567.

SEQ ID NO: 9570 represents the cDNA sequence for clone AI254569.

SEQ ID NO: 9571 represents the cDNA sequence for clone AI254573.

SEQ ID NO: 9572 represents the cDNA sequence for clone AI254582.

SEQ ID NO: 9573 represents the cDNA sequence for clone AI254586.

SEQ ID NO: 9574 represents the cDNA sequence for clone AI254588.

SEQ ID NO: 9575 represents the cDNA sequence for clone AI254589.

SEQ ID NO: 9576 represents the cDNA sequence for clone AI254597.

SEQ ID NO: 9577 represents the cDNA sequence for clone AI254602.

SEQ ID NO: 9578 represents the cDNA sequence for clone AI254609.

SEQ ID NO: 9579 represents the cDNA sequence for clone AI254618.

SEQ ID NO: 9580 represents the cDNA sequence for clone AI254622.

SEQ ID NO: 9581 represents the cDNA sequence for clone AI254625.

SEQ ID NO: 9582 represents the cDNA sequence for clone AI254626.

SEQ ID NO: 9583 represents the cDNA sequence for clone AI254627.

SEQ ID NO: 9584 represents the cDNA sequence for clone AI254633.

SEQ ID NO: 9585 represents the cDNA sequence for clone AI254640.

SEQ ID NO: 9586 represents the cDNA sequence for clone AI254644.

SEQ ID NO: 9587 represents the cDNA sequence for clone AI254651.

SEQ ID NO: 9588 represents the cDNA sequence for clone AI254657.

SEQ ID NO: 9589 represents the cDNA sequence for clone AI254658.

SEQ ID NO: 9590 represents the cDNA sequence for clone AI254660.

SEQ ID NO: 9591 represents the cDNA sequence for clone AI254669.

SEQ ID NO: 9592 represents the cDNA sequence for clone AI254673.

SEQ ID NO: 9593 represents the cDNA sequence for clone AI254674.

SEQ ID NO: 9594 represents the cDNA sequence for clone AI254676.

SEQ ID NO: 9595 represents the cDNA sequence for clone AI254682.

SEQ ID NO: 9596 represents the cDNA sequence for clone AI254684.

SEQ ID NO: 9597 represents the cDNA sequence for clone AI254692.

SEQ ID NO: 9598 represents the cDNA sequence for clone AI254694.

SEQ ID NO: 9599 represents the cDNA sequence for clone AI254696.

SEQ ID NO: 9600 represents the cDNA sequence for clone AI254763.

SEQ ID NO: 9601 represents the cDNA sequence for clone AI254765.

SEQ ID NO: 9602 represents the cDNA sequence for clone AI254767.

SEQ ID NO: 9603 represents the cDNA sequence for clone AI254775.

SEQ ID NO: 9604 represents the cDNA sequence for clone AI254777.

SEQ ID NO: 9605 represents the cDNA sequence for clone AI254781.

SEQ ID NO: 9606 represents the cDNA sequence for clone AI254786.

SEQ ID NO: 9607 represents the cDNA sequence for clone AI254787.

SEQ ID NO: 9608 represents the cDNA sequence for clone AI254791.

SEQ ID NO: 9609 represents the cDNA sequence for clone AI254793.

SEQ ID NO: 9610 represents the cDNA sequence for clone AI254798.

SEQ ID NO: 9611 represents the cDNA sequence for clone AI254810.

SEQ ID NO: 9612 represents the cDNA sequence for clone AI254812.

SEQ ID NO: 9613 represents the cDNA sequence for clone AI254813.

SEQ ID NO: 9614 represents the cDNA sequence for clone AI254817.

SEQ ID NO: 9615 represents the cDNA sequence for clone AI254819.

SEQ ID NO: 9616 represents the cDNA sequence for clone AI254822.

SEQ ID NO: 9617 represents the cDNA sequence for clone AI254831.

SEQ ID NO: 9618 represents the cDNA sequence for clone AI254846.

SEQ ID NO: 9619 represents the cDNA sequence for clone AI254849.

SEQ ID NO: 9620 represents the cDNA sequence for clone AI254866.

SEQ ID NO: 9621 represents the cDNA sequence for clone AI254871.

SEQ ID NO: 9622 represents the cDNA sequence for clone AI254872.

SEQ ID NO: 9623 represents the cDNA sequence for clone AI254873.

SEQ ID NO: 9624 represents the cDNA sequence for clone AI254897.

SEQ ID NO: 9625 represents the cDNA sequence for clone AI254898.

SEQ ID NO: 9626 represents the cDNA sequence for clone AI254906.

SEQ ID NO: 9627 represents the cDNA sequence for clone AI254907.

SEQ ID NO: 9628 represents the cDNA sequence for clone AI254909.

SEQ ID NO: 9629 represents the cDNA sequence for clone AI254910.

SEQ ID NO: 9630 represents the cDNA sequence for clone AI254913.

SEQ ID NO: 9631 represents the cDNA sequence for clone AI254914.

SEQ ID NO: 9632 represents the cDNA sequence for clone AI254916.

SEQ ID NO: 9633 represents the cDNA sequence for clone AI254921.

SEQ ID NO: 9634 represents the cDNA sequence for clone AI254926.

SEQ ID NO: 9635 represents the cDNA sequence for clone AI254927.

SEQ ID NO: 9636 represents the cDNA sequence for clone AI254928.

SEQ ID NO: 9637 represents the cDNA sequence for clone AI254929.

SEQ ID NO: 9638 represents the cDNA sequence for clone AI254935.

SEQ ID NO: 9639 represents the cDNA sequence for clone AI254937.

SEQ ID NO: 9640 represents the cDNA sequence for clone AI254938.

SEQ ID NO: 9641 represents the cDNA sequence for clone AI254939.

SEQ ID NO: 9642 represents the cDNA sequence for clone AI254941.

SEQ ID NO: 9643 represents the cDNA sequence for clone AI254943.

SEQ ID NO: 9644 represents the cDNA sequence for clone AI254952.

SEQ ID NO: 9645 represents the cDNA sequence for clone AI254953.

SEQ ID NO: 9646 represents the cDNA sequence for clone AI254955.

SEQ ID NO: 9647 represents the cDNA sequence for clone AI254958.

SEQ ID NO: 9648 represents the cDNA sequence for clone AI254963.

SEQ ID NO: 9649 represents the cDNA sequence for clone AI254969.

SEQ ID NO: 9650 represents the cDNA sequence for clone AI254973.

SEQ ID NO: 9651 represents the cDNA sequence for clone AI254975.

SEQ ID NO: 9652 represents the cDNA sequence for clone AI254976.

SEQ ID NO: 9653 represents the cDNA sequence for clone AI254978.

SEQ ID NO: 9654 represents the cDNA sequence for clone AI254982.

SEQ ID NO: 9655 represents the cDNA sequence for clone AI254986.

SEQ ID NO: 9656 represents the cDNA sequence for clone AI254989.

SEQ ID NO: 9657 represents the cDNA sequence for clone AI254999.

SEQ ID NO: 9658 represents the cDNA sequence for clone AI255003.

SEQ ID NO: 9659 represents the cDNA sequence for clone AI255005.

SEQ ID NO: 9660 represents the cDNA sequence for clone AI255012.

SEQ ID NO: 9661 represents the cDNA sequence for clone AI255019.

SEQ ID NO: 9662 represents the cDNA sequence for clone AI255023.

SEQ ID NO: 9663 represents the cDNA sequence for clone AI255029.

SEQ ID NO: 9664 represents the cDNA sequence for clone AI255034.

SEQ ID NO: 9665 represents the cDNA sequence for clone AI255037.

SEQ ID NO: 9666 represents the cDNA sequence for clone AI255049.

SEQ ID NO: 9667 represents the cDNA sequence for clone AI255050.

SEQ ID NO: 9668 represents the cDNA sequence for clone AI255053.

SEQ ID NO: 9669 represents the cDNA sequence for clone AI255058.

SEQ ID NO: 9670 represents the cDNA sequence for clone AI255059.

SEQ ID NO: 9671 represents the cDNA sequence for clone AI255062.

SEQ ID NO: 9672 represents the cDNA sequence for clone AI255064.

SEQ ID NO: 9673 represents the cDNA sequence for clone AI255074.

SEQ ID NO: 9674 represents the cDNA sequence for clone AI255076.

SEQ ID NO: 9675 represents the cDNA sequence for clone AI255078.

SEQ ID NO: 9676 represents the cDNA sequence for clone AI255080.

SEQ ID NO: 9677 represents the cDNA sequence for clone AI255082.

SEQ ID NO: 9678 represents the cDNA sequence for clone AI255085.

SEQ ID NO: 9679 represents the cDNA sequence for clone AI255089.

SEQ ID NO: 9680 represents the cDNA sequence for clone AI255090.

SEQ ID NO: 9681 represents the cDNA sequence for clone AI255095.

SEQ ID NO: 9682 represents the cDNA sequence for clone AI255101.

SEQ ID NO: 9683 represents the cDNA sequence for clone AI255107.

SEQ ID NO: 9684 represents the cDNA sequence for clone AI255108.

SEQ ID NO: 9685 represents the cDNA sequence for clone AI255115.

SEQ ID NO: 9686 represents the cDNA sequence for clone AI255120.

SEQ ID NO: 9687 represents the cDNA sequence for clone AI255122.

SEQ ID NO: 9688 represents the cDNA sequence for clone AI255127.

SEQ ID NO: 9689 represents the cDNA sequence for clone AI255133.

SEQ ID NO: 9690 represents the cDNA sequence for clone AI255143.

SEQ ID NO: 9691 represents the cDNA sequence for clone AI255145.

SEQ ID NO: 9692 represents the cDNA sequence for clone AI263659.

SEQ ID NO: 9693 represents the cDNA sequence for clone AI263665.

SEQ ID NO: 9694 represents the cDNA sequence for clone AI263669.

SEQ ID NO: 9695 represents the cDNA sequence for clone AI263673.

SEQ ID NO: 9696 represents the cDNA sequence for clone AI263674.

SEQ ID NO: 9697 represents the cDNA sequence for clone AI263687.

SEQ ID NO: 9698 represents the cDNA sequence for clone AI264833.

SEQ ID NO: 9699 represents the cDNA sequence for clone AI264835.

SEQ ID NO: 9700 represents the cDNA sequence for clone AI264845.

SEQ ID NO: 9701 represents the cDNA sequence for clone AI264852.

SEQ ID NO: 9702 represents the cDNA sequence for clone AI264856.

SEQ ID NO: 9703 represents the cDNA sequence for clone AI264935.

SEQ ID NO: 9704 represents the cDNA sequence for clone AI264941.

SEQ ID NO: 9705 represents the cDNA sequence for clone AI264951.

SEQ ID NO: 9706 represents the cDNA sequence for clone AI264966.

SEQ ID NO: 9707 represents the cDNA sequence for clone AI265778.

SEQ ID NO: 9708 represents the cDNA sequence for clone AI265784.

SEQ ID NO: 9709 represents the cDNA sequence for clone AI265791.

SEQ ID NO: 9710 represents the cDNA sequence for clone AI265799.

SEQ ID NO: 9711 represents the cDNA sequence for clone AI265818.

SEQ ID NO: 9712 represents the cDNA sequence for clone AI265819.

SEQ ID NO: 9713 represents the cDNA sequence for clone AI265821.

SEQ ID NO: 9714 represents the cDNA sequence for clone AI265803.

SEQ ID NO: 9715 represents the cDNA sequence for clone AI265824.

SEQ ID NO: 9716 represents the cDNA sequence for clone AI265902.

SEQ ID NO: 9717 represents the cDNA sequence for clone AI265904.

SEQ ID NO: 9718 represents the cDNA sequence for clone AI265908.

SEQ ID NO: 9719 represents the cDNA sequence for clone AI265909.

SEQ ID NO: 9720 represents the cDNA sequence for clone AI265914.

SEQ ID NO: 9721 represents the cDNA sequence for clone AI266250.

SEQ ID NO: 9722 represents the cDNA sequence for clone AI266254.

SEQ ID NO: 9723 represents the cDNA sequence for clone AI266255.

SEQ ID NO: 9724 represents the cDNA sequence for clone AI266271.

SEQ ID NO: 9725 represents the cDNA sequence for clone AI266275.

SEQ ID NO: 9726 represents the cDNA sequence for clone AI266280.

SEQ ID NO: 9727 represents the cDNA sequence for clone AI266282.

SEQ ID NO: 9728 represents the cDNA sequence for clone AI270142.

SEQ ID NO: 9729 represents the cDNA sequence for clone AI270143.

SEQ ID NO: 9730 represents the cDNA sequence for clone AI270144.

SEQ ID NO: 9731 represents the cDNA sequence for clone AI270147.

SEQ ID NO: 9732 represents the cDNA sequence for clone AI270149.

SEQ ID NO: 9733 represents the cDNA sequence for clone AI270160.

SEQ ID NO: 9734 represents the cDNA sequence for clone AI270163.

SEQ ID NO: 9735 represents the cDNA sequence for clone AI270747.

SEQ ID NO: 9736 represents the cDNA sequence for clone AI270754.

SEQ ID NO: 9737 represents the cDNA sequence for clone AI270755.

SEQ ID NO: 9738 represents the cDNA sequence for clone AI270758.

SEQ ID NO: 9739 represents the cDNA sequence for clone AI270759.

SEQ ID NO: 9740 represents the cDNA sequence for clone AI270760.

SEQ ID NO: 9741 represents the cDNA sequence for clone AI270768.

SEQ ID NO: 9742 represents the cDNA sequence for clone AI270779.

SEQ ID NO: 9743 represents the cDNA sequence for clone AI270784.

SEQ ID NO: 9744 represents the cDNA sequence for clone AI270849.

SEQ ID NO: 9745 represents the cDNA sequence for clone AI270864.

SEQ ID NO: 9746 represents the cDNA sequence for clone AI270877.

SEQ ID NO: 9747 represents the cDNA sequence for clone AI270878.

SEQ ID NO: 9748 represents the cDNA sequence for clone AI270891.

SEQ ID NO: 9749 represents the cDNA sequence for clone AI270892.

SEQ ID NO: 9750 represents the cDNA sequence for clone AI270899.

SEQ ID NO: 9751 represents the cDNA sequence for clone AI270906.

SEQ ID NO: 9752 represents the cDNA sequence for clone AI271007.

SEQ ID NO: 9753 represents the cDNA sequence for clone AI271016.

SEQ ID NO: 9754 represents the cDNA sequence for clone AI271079.

SEQ ID NO: 9755 represents the cDNA sequence for clone AI271096.

SEQ ID NO: 9756 represents the cDNA sequence for clone AI271118.

SEQ ID NO: 9757 represents the cDNA sequence for clone AI271124.

SEQ ID NO: 9758 represents the cDNA sequence for clone AI271164.

SEQ ID NO: 9759 represents the cDNA sequence for clone AI271175.

SEQ ID NO: 9760 represents the cDNA sequence for clone AI271176.

SEQ ID NO: 9761 represents the cDNA sequence for clone AI271203.

SEQ ID NO: 9762 represents the cDNA sequence for clone AI271300.

SEQ ID NO: 9763 represents the cDNA sequence for clone AI271313.

SEQ ID NO: 9764 represents the cDNA sequence for clone AI273774.

SEQ ID NO: 9765 represents the cDNA sequence for clone AI273776.

SEQ ID NO: 9766 represents the cDNA sequence for clone AI273786.

SEQ ID NO: 9767 represents the cDNA sequence for clone AI273793.

SEQ ID NO: 9768 represents the cDNA sequence for clone AI273795.

SEQ ID NO: 9769 represents the cDNA sequence for clone AI273800.

SEQ ID NO: 9770 represents the cDNA sequence for clone AI273805.

SEQ ID NO: 9771 represents the cDNA sequence for clone AI273810.

SEQ ID NO: 9772 represents the cDNA sequence for clone AI273812.

SEQ ID NO: 9773 represents the cDNA sequence for clone AI273821.

SEQ ID NO: 9774 represents the cDNA sequence for clone AI273830.

SEQ ID NO: 9775 represents the cDNA sequence for clone AI273834.

SEQ ID NO: 9776 represents the cDNA sequence for clone AI273835.

SEQ ID NO: 9777 represents the cDNA sequence for clone AI273840.

SEQ ID NO: 9778 represents the cDNA sequence for clone AI273842.

SEQ ID NO: 9779 represents the cDNA sequence for clone AI273858.

SEQ ID NO: 9780 represents the cDNA sequence for clone AI273862.

SEQ ID NO: 9781 represents the cDNA sequence for clone AI273875.

SEQ ID NO: 9782 represents the cDNA sequence for clone AI273878.

SEQ ID NO: 9783 represents the cDNA sequence for clone AI273880.

SEQ ID NO: 9784 represents the cDNA sequence for clone AI273889.

SEQ ID NO: 9785 represents the cDNA sequence for clone AI273890.

SEQ ID NO: 9786 represents the cDNA sequence for clone AI273897.

SEQ ID NO: 9787 represents the cDNA sequence for clone AI273899.

SEQ ID NO: 9788 represents the cDNA sequence for clone AI275340.

SEQ ID NO: 9789 represents the cDNA sequence for clone AI275349.

SEQ ID NO: 9790 represents the cDNA sequence for clone AI275357.

SEQ ID NO: 9791 represents the cDNA sequence for clone AI275361.

SEQ ID NO: 9792 represents the cDNA sequence for clone AI279381.

SEQ ID NO: 9793 represents the cDNA sequence for clone AI279382.

SEQ ID NO: 9794 represents the cDNA sequence for clone AI279387.

SEQ ID NO: 9795 represents the cDNA sequence for clone AI279388.

SEQ ID NO: 9796 represents the cDNA sequence for clone AI279389.

SEQ ID NO: 9797 represents the cDNA sequence for clone AI279390.

SEQ ID NO: 9798 represents the cDNA sequence for clone AI279392.

SEQ ID NO: 9799 represents the cDNA sequence for clone AI279399.

SEQ ID NO: 9800 represents the cDNA sequence for clone AI279404.

SEQ ID NO: 9801 represents the cDNA sequence for clone AI279405.

SEQ ID NO: 9802 represents the cDNA sequence for clone AI279408.

SEQ ID NO: 9803 represents the cDNA sequence for clone AI280487.

SEQ ID NO: 9804 represents the cDNA sequence for clone AI280489.

SEQ ID NO: 9805 represents the cDNA sequence for clone AI280491.

SEQ ID NO: 9806 represents the cDNA sequence for clone AI280493.

SEQ ID NO: 9807 represents the cDNA sequence for clone AI280508.

SEQ ID NO: 9808 represents the cDNA sequence for clone AI280509.

SEQ ID NO: 9809 represents the cDNA sequence for clone AI280511.

SEQ ID NO: 9810 represents the cDNA sequence for clone AI280512.

SEQ ID NO: 9811 represents the cDNA sequence for clone AI280522.

SEQ ID NO: 9812 represents the cDNA sequence for clone AI280524.

SEQ ID NO: 9813 represents the cDNA sequence for clone AI280528.

SEQ ID NO: 9814 represents the cDNA sequence for clone AI280530.

SEQ ID NO: 9815 represents the cDNA sequence for clone AI280533.

SEQ ID NO: 9816 represents the cDNA sequence for clone AI280534.

SEQ ID NO: 9817 represents the cDNA sequence for clone AI280535.

SEQ ID NO: 9818 represents the cDNA sequence for clone AI280544.

SEQ ID NO: 9819 represents the cDNA sequence for clone AI280566.

SEQ ID NO: 9820 represents the cDNA sequence for clone AI280567.

SEQ ID NO: 9821 represents the cDNA sequence for clone AI280571.

SEQ ID NO: 9822 represents the cDNA sequence for clone AI280608.

SEQ ID NO: 9823 represents the cDNA sequence for clone AI280612.

SEQ ID NO: 9824 represents the cDNA sequence for clone AI280614.

SEQ ID NO: 9825 represents the cDNA sequence for clone AI280620.

SEQ ID NO: 9826 represents the cDNA sequence for clone AI280622.

SEQ ID NO: 9827 represents the cDNA sequence for clone AI280624.

SEQ ID NO: 9828 represents the cDNA sequence for clone AI280626.

SEQ ID NO: 9829 represents the cDNA sequence for clone AI280628.

SEQ ID NO: 9830 represents the cDNA sequence for clone AI280630.

SEQ ID NO: 9831 represents the cDNA sequence for clone AI280634.

SEQ ID NO: 9832 represents the cDNA sequence for clone AI280639.

SEQ ID NO: 9833 represents the cDNA sequence for clone AI280641.

SEQ ID NO: 9834 represents the cDNA sequence for clone AI280646.

SEQ ID NO: 9835 represents the cDNA sequence for clone AI280649.

SEQ ID NO: 9836 represents the cDNA sequence for clone AI280656.

SEQ ID NO: 9837 represents the cDNA sequence for clone AI280668.

SEQ ID NO: 9838 represents the cDNA sequence for clone AI280674.

SEQ ID NO: 9839 represents the cDNA sequence for clone AI280675.

SEQ ID NO: 9840 represents the cDNA sequence for clone AI283279.

SEQ ID NO: 9841 represents the cDNA sequence for clone AI283286.

SEQ ID NO: 9842 represents the cDNA sequence for clone AI283289.

SEQ ID NO: 9843 represents the cDNA sequence for clone AI283290.

SEQ ID NO: 9844 represents the cDNA sequence for clone AI283295.

SEQ ID NO: 9845 represents the cDNA sequence for clone AI283300.

SEQ ID NO: 9846 represents the cDNA sequence for clone AI283302.

SEQ ID NO: 9847 represents the cDNA sequence for clone AI283303.

SEQ ID NO: 9848 represents the cDNA sequence for clone AI283304.

SEQ ID NO: 9849 represents the cDNA sequence for clone AI283308.

SEQ ID NO: 9850 represents the cDNA sequence for clone AI283310.

SEQ ID NO: 9851 represents the cDNA sequence for clone AI283312.

SEQ ID NO: 9852 represents the cDNA sequence for clone AI283317.

SEQ ID NO: 9853 represents the cDNA sequence for clone AI283319.

SEQ ID NO: 9854 represents the cDNA sequence for clone AI283322.

SEQ ID NO: 9855 represents the cDNA sequence for clone AI283324.

SEQ ID NO: 9856 represents the cDNA sequence for clone AI283328.

SEQ ID NO: 9857 represents the cDNA sequence for clone AI283330.

SEQ ID NO: 9858 represents the cDNA sequence for clone AI283331.

SEQ ID NO: 9859 represents the cDNA sequence for clone AI283332.

SEQ ID NO: 9860 represents the cDNA sequence for clone AI283335.

SEQ ID NO: 9861 represents the cDNA sequence for clone AI283336.

SEQ ID NO: 9862 represents the cDNA sequence for clone AI283352.

SEQ ID NO: 9863 represents the cDNA sequence for clone AI283353.

SEQ ID NO: 9864 represents the cDNA sequence for clone AI283365.

SEQ ID NO: 9865 represents the cDNA sequence for clone AI283370.

SEQ ID NO: 9866 represents the cDNA sequence for clone AI283374.

SEQ ID NO: 9867 represents the cDNA sequence for clone AI284589.

SEQ ID NO: 9868 represents the cDNA sequence for clone AI284599.

SEQ ID NO: 9869 represents the cDNA sequence for clone AI284647.

SEQ ID NO: 9870 represents the cDNA sequence for clone AI284654.

SEQ ID NO: 9871 represents the cDNA sequence for clone AI284660.

SEQ ID NO: 9872 represents the cDNA sequence for clone AI284670.

SEQ ID NO: 9873 represents the cDNA sequence for clone AI284713.

SEQ ID NO: 9874 represents the cDNA sequence for clone AI284718.

SEQ ID NO: 9875 represents the cDNA sequence for clone AI284724.

SEQ ID NO: 9876 represents the cDNA sequence for clone AI284727.

SEQ ID NO: 9877 represents the cDNA sequence for clone AI284731.

SEQ ID NO: 9878 represents the cDNA sequence for clone AI284776.

SEQ ID NO: 9879 represents the cDNA sequence for clone AI284786.

SEQ ID NO: 9880 represents the cDNA sequence for clone AI284790.

SEQ ID NO: 9881 represents the cDNA sequence for clone AI284793.

SEQ ID NO: 9882 represents the cDNA sequence for clone AI284801.

SEQ ID NO: 9883 represents the cDNA sequence for clone AI284847.

SEQ ID NO: 9884 represents the cDNA sequence for clone AI284849.

SEQ ID NO: 9885 represents the cDNA sequence for clone AI284850.

SEQ ID NO: 9886 represents the cDNA sequence for clone AI284861.

SEQ ID NO: 9887 represents the cDNA sequence for clone AI284862.

SEQ ID NO: 9888 represents the cDNA sequence for clone AI284866.

SEQ ID NO: 9889 represents the cDNA sequence for clone AI284869.

SEQ ID NO: 9890 represents the cDNA sequence for clone AI284870.

SEQ ID NO: 9891 represents the cDNA sequence for clone AI284874.

SEQ ID NO: 9892 represents the cDNA sequence for clone AI287698.

SEQ ID NO: 9893 represents the cDNA sequence for clone AI287702.

SEQ ID NO: 9894 represents the cDNA sequence for clone AI287703.

SEQ ID NO: 9895 represents the cDNA sequence for clone AI287705.

SEQ ID NO: 9896 represents the cDNA sequence for clone AI287717.

SEQ ID NO: 9897 represents the cDNA sequence for clone AI287725.

SEQ ID NO: 9898 represents the cDNA sequence for clone AI287897.

SEQ ID NO: 9899 represents the cDNA sequence for clone AI287898.

SEQ ID NO: 9900 represents the cDNA sequence for clone AI287907.

SEQ ID NO: 9901 represents the cDNA sequence for clone AI287911.

SEQ ID NO: 9902 represents the cDNA sequence for clone AI287912.

SEQ ID NO: 9903 represents the cDNA sequence for clone AI287917.

SEQ ID NO: 9904 represents the cDNA sequence for clone AI287920.

SEQ ID NO: 9905 represents the cDNA sequence for clone AI287921.

SEQ ID NO: 9906 represents the cDNA sequence for clone AI305913.

SEQ ID NO: 9907 represents the cDNA sequence for clone AI305996.

SEQ ID NO: 9908 represents the cDNA sequence for clone AI306003.

SEQ ID NO: 9909 represents the cDNA sequence for clone AI306018.

SEQ ID NO: 9910 represents the cDNA sequence for clone AI306022.

SEQ ID NO: 9911 represents the cDNA sequence for clone AI306211.

SEQ ID NO: 9912 represents the cDNA sequence for clone AI306310.

SEQ ID NO: 9913 represents the cDNA sequence for clone AI306323.

SEQ ID NO: 9914 represents the cDNA sequence for clone AI306325.

SEQ ID NO: 9915 represents the cDNA sequence for clone AI306331.

SEQ ID NO: 9916 represents the cDNA sequence for clone AI306966.

SEQ ID NO: 9917 represents the cDNA sequence for clone AI306979.

SEQ ID NO: 9918 represents the cDNA sequence for clone AI306990.

SEQ ID NO: 9919 represents the cDNA sequence for clone AI307037.

SEQ ID NO: 9920 represents the cDNA sequence for clone AI307084.

SEQ ID NO: 9921 represents the cDNA sequence for clone AI307086.

SEQ ID NO: 9922 represents the cDNA sequence for clone AI307094.

SEQ ID NO: 9923 represents the cDNA sequence for clone AI305310.

SEQ ID NO: 9924 represents the cDNA sequence for clone AI305393.

SEQ ID NO: 9925 represents the cDNA sequence for clone AI305502.

SEQ ID NO: 9926 represents the cDNA sequence for clone AI305507.

SEQ ID NO: 9927 represents the cDNA sequence for clone AI305509.

SEQ ID NO: 9928 represents the cDNA sequence for clone AI305623.

SEQ ID NO: 9929 represents the cDNA sequence for clone AI305644.

SEQ ID NO: 9930 represents the cDNA sequence for clone AI305766.

SEQ ID NO: 9931 represents the cDNA sequence for clone AI307407.

SEQ ID NO: 9932 represents the cDNA sequence for clone AI307410.

SEQ ID NO: 9933 represents the cDNA sequence for clone AI307416.

SEQ ID NO: 9934 represents the cDNA sequence for clone AI307421.

SEQ ID NO: 9935 represents the cDNA sequence for clone AI307424.

SEQ ID NO: 9936 represents the cDNA sequence for clone AI307426.

SEQ ID NO: 9937 represents the cDNA sequence for clone AI307430.

SEQ ID NO: 9938 represents the cDNA sequence for clone AI307433.

SEQ ID NO: 9939 represents the cDNA sequence for clone AI307602.

SEQ ID NO: 9940 represents the cDNA sequence for clone AI308002.

SEQ ID NO: 9941 represents the cDNA sequence for clone AI308006.

SEQ ID NO: 9942 represents the cDNA sequence for clone AI308015.

SEQ ID NO: 9943 represents the cDNA sequence for clone AI308041.

SEQ ID NO: 9944 represents the cDNA sequence for clone AI318117.

SEQ ID NO: 9945 represents the cDNA sequence for clone AI318121.

SEQ ID NO: 9946 represents the cDNA sequence for clone AI318142.

SEQ ID NO: 9947 represents the cDNA sequence for clone AI318162.

SEQ ID NO: 9948 represents the cDNA sequence for clone AI318167.

SEQ ID NO: 9949 represents the cDNA sequence for clone AI318168.

SEQ ID NO: 9950 represents the cDNA sequence for clone AI318173.

SEQ ID NO: 9951 represents the cDNA sequence for clone AI318174.

SEQ ID NO: 9952 represents the cDNA sequence for clone AI318176.

SEQ ID NO: 9953 represents the cDNA sequence for clone AI318183.

SEQ ID NO: 9954 represents the cDNA sequence for clone AI318250.

SEQ ID NO: 9955 represents the cDNA sequence for clone AI318251.

SEQ ID NO: 9956 represents the cDNA sequence for clone AI318253.

SEQ ID NO: 9957 represents the cDNA sequence for clone AI318255.

SEQ ID NO: 9958 represents the cDNA sequence for clone AI318282.

SEQ ID NO: 9959 represents the cDNA sequence for clone AI318285.

SEQ ID NO: 9960 represents the cDNA sequence for clone AI318286.

SEQ ID NO: 9961 represents the cDNA sequence for clone AI318288.

SEQ ID NO: 9962 represents the cDNA sequence for clone AI318289.

SEQ ID NO: 9963 represents the cDNA sequence for clone AI318291.

SEQ ID NO: 9964 represents the cDNA sequence for clone AI318294.

SEQ ID NO: 9965 represents the cDNA sequence for clone AI318304.

SEQ ID NO: 9966 represents the cDNA sequence for clone AI318308.

SEQ ID NO: 9967 represents the cDNA sequence for clone AI318549.

SEQ ID NO: 9968 represents the cDNA sequence for clone AI318551.

SEQ ID NO: 9969 represents the cDNA sequence for clone AI318570.

SEQ ID NO: 9970 represents the cDNA sequence for clone AI318571.

SEQ ID NO: 9971 represents the cDNA sequence for clone AI318604.

SEQ ID NO: 9972 represents the cDNA sequence for clone AI318614.

SEQ ID NO: 9973 represents the cDNA sequence for clone AI318624.

SEQ ID NO: 9974 represents the cDNA sequence for clone AI318627.

SEQ ID NO: 9975 represents the cDNA sequence for clone AI344787.

SEQ ID NO: 9976 represents the cDNA sequence for clone AI344805.

SEQ ID NO: 9977 represents the cDNA sequence for clone AI344896.

SEQ ID NO: 9978 represents the cDNA sequence for clone AI344909.

SEQ ID NO: 9979 represents the cDNA sequence for clone AI344912.

SEQ ID NO: 9980 represents the cDNA sequence for clone AI344917.

SEQ ID NO: 9981 represents the cDNA sequence for clone AI354410.

SEQ ID NO: 9982 represents the cDNA sequence for clone AI354411.

SEQ ID NO: 9983 represents the cDNA sequence for clone AI354414.

SEQ ID NO: 9984 represents the cDNA sequence for clone AI354415.

SEQ ID NO: 9985 represents the cDNA sequence for clone AI354419.

SEQ ID NO: 9986 represents the cDNA sequence for clone AI354423.

SEQ ID NO: 9987 represents the cDNA sequence for clone AI354425.

SEQ ID NO: 9988 represents the cDNA sequence for clone AI354426.

SEQ ID NO: 9989 represents the cDNA sequence for clone AI354431.

SEQ ID NO: 9990 represents the cDNA sequence for clone AI354434.

SEQ ID NO: 9991 represents the cDNA sequence for clone AI354435.

SEQ ID NO: 9992 represents the cDNA sequence for clone AI354444.

SEQ ID NO: 9993 represents the cDNA sequence for clone AI354451.

SEQ ID NO: 9994 represents the cDNA sequence for clone AI354453.

SEQ ID NO: 9995 represents the cDNA sequence for clone AI354458.

SEQ ID NO: 9996 represents the cDNA sequence for clone AI354459.

SEQ ID NO: 9997 represents the cDNA sequence for clone AI354461.

SEQ ID NO: 9998 represents the cDNA sequence for clone AI354989.

SEQ ID NO: 9999 represents the cDNA sequence for clone AI354991.

SEQ ID NO: 10000 represents the cDNA sequence for clone AI354993.

SEQ ID NO: 10001 represents the cDNA sequence for clone AI354996.

SEQ ID NO: 10002 represents the cDNA sequence for clone AI354997.

SEQ ID NO: 10003 represents the cDNA sequence for clone AI355000.

SEQ ID NO: 10004 represents the cDNA sequence for clone AI355001.

SEQ ID NO: 10005 represents the cDNA sequence for clone AI355004.

SEQ ID NO: 10006 represents the cDNA sequence for clone AI355005.

SEQ ID NO: 10007 represents the cDNA sequence for clone AI355007.

SEQ ID NO: 10008 represents the cDNA sequence for clone AI355009.

SEQ ID NO: 10009 represents the cDNA sequence for clone AI355012.

SEQ ID NO: 10010 represents the cDNA sequence for clone AI355055.

SEQ ID NO: 10011 represents the cDNA sequence for clone AI355056.

SEQ ID NO: 10012 represents the cDNA sequence for clone AI355057.

SEQ ID NO: 10013 represents the cDNA sequence for clone AI355063.

SEQ ID NO: 10014 represents the cDNA sequence for clone AI355069.

SEQ ID NO: 10015 represents the cDNA sequence for clone AI355070.

SEQ ID NO: 10016 represents the cDNA sequence for clone AI355075.

SEQ ID NO: 10017 represents the cDNA sequence for clone AI355077.

SEQ ID NO: 10018 represents the cDNA sequence for clone AI355483.

SEQ ID NO: 10019 represents the cDNA sequence for clone AI355489.

SEQ ID NO: 10020 represents the cDNA sequence for clone AI355519.

SEQ ID NO: 10021 represents the cDNA sequence for clone AI355520.

SEQ ID NO: 10022 represents the cDNA sequence for clone AI355521.

SEQ ID NO: 10023 represents the cDNA sequence for clone AI355523.

SEQ ID NO: 10024 represents the cDNA sequence for clone AI355540.

SEQ ID NO: 10025 represents the cDNA sequence for clone AI355541.

SEQ ID NO: 10026 represents the cDNA sequence for clone AI355546.

SEQ ID NO: 10027 represents the cDNA sequence for clone AI355547.

SEQ ID NO: 10028 represents the cDNA sequence for clone AI356590.

SEQ ID NO: 10029 represents the cDNA sequence for clone AI356595.

SEQ ID NO: 10030 represents the cDNA sequence for clone AI356599.

SEQ ID NO: 10031 represents the cDNA sequence for clone AI356605.

SEQ ID NO: 10032 represents the cDNA sequence for clone AI357533.

SEQ ID NO: 10033 represents the cDNA sequence for clone AI357534.

SEQ ID NO: 10034 represents the cDNA sequence for clone AI357539.

SEQ ID NO: 10035 represents the cDNA sequence for clone AI357541.

SEQ ID NO: 10036 represents the cDNA sequence for clone AI357542.

SEQ ID NO: 10037 represents the cDNA sequence for clone AI357543.

SEQ ID NO: 10038 represents the cDNA sequence for clone AI357545.

SEQ ID NO: 10039 represents the cDNA sequence for clone AI357547.

SEQ ID NO: 10040 represents the cDNA sequence for clone AI357551.

SEQ ID NO: 10041 represents the cDNA sequence for clone AI357556.

SEQ ID NO: 10042 represents the cDNA sequence for clone AI357561.

SEQ ID NO: 10043 represents the cDNA sequence for clone AI357600.

SEQ ID NO: 10044 represents the cDNA sequence for clone AI357605.

SEQ ID NO: 10045 represents the cDNA sequence for clone AI357614.

SEQ ID NO: 10046 represents the cDNA sequence for clone AI357620.

SEQ ID NO: 10047 represents the cDNA sequence for clone AI357622.

SEQ ID NO: 10048 represents the cDNA sequence for clone AI357623.

SEQ ID NO: 10049 represents the cDNA sequence for clone AI357626.

SEQ ID NO: 10050 represents the cDNA sequence for clone AI357628.

SEQ ID NO: 10051 represents the cDNA sequence for clone AI357630.

SEQ ID NO: 10052 represents the cDNA sequence for clone AI357673.

SEQ ID NO: 10053 represents the cDNA sequence for clone AI357676.

SEQ ID NO: 10054 represents the cDNA sequence for clone AI357688.

SEQ ID NO: 10055 represents the cDNA sequence for clone AI357690.

SEQ ID NO: 10056 represents the cDNA sequence for clone AI357691.

SEQ ID NO: 10057 represents the cDNA sequence for clone AI364753.

SEQ ID NO: 10058 represents the cDNA sequence for clone AI364758.

SEQ ID NO: 10059 represents the cDNA sequence for clone AI364759.

SEQ ID NO: 10060 represents the cDNA sequence for clone AI364761.

SEQ ID NO: 10061 represents the cDNA sequence for clone AI364772.

SEQ ID NO: 10062 represents the cDNA sequence for clone AI364775.

SEQ ID NO: 10063 represents the cDNA sequence for clone AI364777.

SEQ ID NO: 10064 represents the cDNA sequence for clone AI364781.

SEQ ID NO: 10065 represents the cDNA sequence for clone AI364789.

SEQ ID NO: 10066 represents the cDNA sequence for clone AI364792.

SEQ ID NO: 10067 represents the cDNA sequence for clone AI364798.

SEQ ID NO: 10068 represents the cDNA sequence for clone AI364800.

SEQ ID NO: 10069 represents the cDNA sequence for clone AI364801.

SEQ ID NO: 10070 represents the cDNA sequence for clone AI364802.

SEQ ID NO: 10071 represents the cDNA sequence for clone AI364803.

SEQ ID NO: 10072 represents the cDNA sequence for clone AI364805.

SEQ ID NO: 10073 represents the cDNA sequence for clone AI364813.

SEQ ID NO: 10074 represents the cDNA sequence for clone AI364815.

SEQ ID NO: 10075 represents the cDNA sequence for clone AI364825.

SEQ ID NO: 10076 represents the cDNA sequence for clone AI364832.

SEQ ID NO: 10077 represents the cDNA sequence for clone AI364837.

SEQ ID NO: 10078 represents the cDNA sequence for clone AI364842.

SEQ ID NO: 10079 represents the cDNA sequence for clone AI364849.

SEQ ID NO: 10080 represents the cDNA sequence for clone AI364850.

SEQ ID NO: 10081 represents the cDNA sequence for clone AI364851.

SEQ ID NO: 10082 represents the cDNA sequence for clone AI364852.

SEQ ID NO: 10083 represents the cDNA sequence for clone AI364854.

SEQ ID NO: 10084 represents the cDNA sequence for clone AI364858.

SEQ ID NO: 10085 represents the cDNA sequence for clone AI364860.

SEQ ID NO: 10086 represents the cDNA sequence for clone AI364862.

SEQ ID NO: 10087 represents the cDNA sequence for clone AI364864.

SEQ ID NO: 10088 represents the cDNA sequence for clone AI364865.

SEQ ID NO: 10089 represents the cDNA sequence for clone AI364875.

SEQ ID NO: 10090 represents the cDNA sequence for clone AI364872.

SEQ ID NO: 10091 represents the cDNA sequence for clone AI364874.

SEQ ID NO: 10092 represents the cDNA sequence for clone AI370141.

SEQ ID NO: 10093 represents the cDNA sequence for clone AI370147.

SEQ ID NO: 10094 represents the cDNA sequence for clone AI370153.

SEQ ID NO: 10095 represents the cDNA sequence for clone AI370155.

SEQ ID NO: 10096 represents the cDNA sequence for clone AI370156.

SEQ ID NO: 10097 represents the cDNA sequence for clone AI370160.

SEQ ID NO: 10098 represents the cDNA sequence for clone AI370162.

SEQ ID NO: 10099 represents the cDNA sequence for clone AI436625.

SEQ ID NO: 10100 represents the cDNA sequence for clone AI436631.

SEQ ID NO: 10101 represents the cDNA sequence for clone AI436632.

SEQ ID NO: 10102 represents the cDNA sequence for clone AI436635.

SEQ ID NO: 10103 represents the cDNA sequence for clone AI436636.

SEQ ID NO: 10104 represents the cDNA sequence for clone AI436638.

SEQ ID NO: 10104 represents the cDNA sequence for clone AI521398.

SEQ ID NO: 10106 represents the cDNA sequence for clone AI521399.

SEQ ID NO: 10107 represents the cDNA sequence for clone AI521401.

SEQ ID NO: 10108 represents the cDNA sequence for clone AI521402.

SEQ ID NO: 10109 represents the cDNA sequence for clone AI521403.

SEQ ID NO: 10110 represents the cDNA sequence for clone AI521408.

SEQ ID NO: 10111 represents the cDNA sequence for clone AI521410.

SEQ ID NO: 10112 represents the cDNA sequence for clone AI521416.

SEQ ID NO: 10113 represents the cDNA sequence for clone AI521417.

SEQ ID NO: 10114 represents the cDNA sequence for clone AI521420.

SEQ ID NO: 10115 represents the cDNA sequence for clone AI521425.

SEQ ID NO: 10116 represents the cDNA sequence for clone AI521430.

SEQ ID NO: 10117 represents the cDNA sequence for clone AI521435.

SEQ ID NO: 10118 represents the cDNA sequence for clone AI521438.

SEQ ID NO: 10119 represents the cDNA sequence for clone AI521443.

SEQ ID NO: 10120 represents the cDNA sequence for clone AI521444.

SEQ ID NO: 10121 represents the cDNA sequence for clone AI521445.

SEQ ID NO: 10122 represents the cDNA sequence for clone AI521447.

SEQ ID NO: 10123 represents the cDNA sequence for clone AI521461.

SEQ ID NO: 10124 represents the cDNA sequence for clone AI521471.

SEQ ID NO: 10125 represents the cDNA sequence for clone AI521472.

SEQ ID NO: 10126 represents the cDNA sequence for clone AI567090.

SEQ ID NO: 10127 represents the cDNA sequence for clone AI567097.

SEQ ID NO: 10128 represents the cDNA sequence for clone AI567106.

SEQ ID NO: 10129 represents the cDNA sequence for clone AI567108.

SEQ ID NO: 10130 represents the cDNA sequence for clone AI567113.

SEQ ID NO: 10131 represents the cDNA sequence for clone AI567114.

SEQ ID NO: 10132 represents the cDNA sequence for clone AI567119.

SEQ ID NO: 10133 represents the cDNA sequence for clone AI567120.

SEQ ID NO: 10134 represents the cDNA sequence for clone AI567129.

SEQ ID NO: 10135 represents the cDNA sequence for clone AI567140.

SEQ ID NO: 10136 represents the cDNA sequence for clone AI567141.

SEQ ID NO: 10137 represents the cDNA sequence for clone AI567142.

SEQ ID NO: 10138 represents the cDNA sequence for clone AI567143.

SEQ ID NO: 10139 represents the cDNA sequence for clone AI567144.

SEQ ID NO: 10140 represents the cDNA sequence for clone AI567149.

SEQ ID NO: 10141 represents the cDNA sequence for clone AI567237.

SEQ ID NO: 10142 represents the cDNA sequence for clone AI567240.

SEQ ID NO: 10143 represents the cDNA sequence for clone AI567241.

SEQ ID NO: 10144 represents the cDNA sequence for clone AI567243.

SEQ ID NO: 10145 represents the cDNA sequence for clone AI567245.

SEQ ID NO: 10146 represents the cDNA sequence for clone AI567247.

SEQ ID NO: 10147 represents the cDNA sequence for clone AI567250.

SEQ ID NO: 10148 represents the cDNA sequence for clone AI567251.

SEQ ID NO: 10149 represents the cDNA sequence for clone AI567255.

SEQ ID NO: 10150 represents the cDNA sequence for clone AI567258.

SEQ ID NO: 10151 represents the cDNA sequence for clone AI567262.

SEQ ID NO: 10152 represents the cDNA sequence for clone AI567263.

SEQ ID NO: 10153 represents the cDNA sequence for clone AI567264.

SEQ ID NO: 10154 represents the cDNA sequence for clone AI567269.

SEQ ID NO: 10155 represents the cDNA sequence for clone AI567274.

SEQ ID NO: 10156 represents the cDNA sequence for clone AI567276.

SEQ ID NO: 10157 represents the cDNA sequence for clone AI567287.

SEQ ID NO: 10158 represents the cDNA sequence for clone AI567290.

SEQ ID NO: 10159 represents the cDNA sequence for clone AI567293.

SEQ ID NO: 10160 represents the cDNA sequence for clone AI567300.

SEQ ID NO: 10161 represents the cDNA sequence for clone AI567821.

SEQ ID NO: 10162 represents the cDNA sequence for clone AI567824.

SEQ ID NO: 10163 represents the cDNA sequence for clone AI567827.

SEQ ID NO: 10164 represents the cDNA sequence for clone AI567829.

SEQ ID NO: 10165 represents the cDNA sequence for clone AI567830.

SEQ ID NO: 10166 represents the cDNA sequence for clone AI567832.

SEQ ID NO: 10167 represents the cDNA sequence for clone AI567833.

SEQ ID NO: 10168 represents the cDNA sequence for clone AI567837.

SEQ ID NO: 10169 represents the cDNA sequence for clone AI567839.

SEQ ID NO: 10170 represents the cDNA sequence for clone AI567847.

SEQ ID NO: 10171 represents the cDNA sequence for clone AI567850.

SEQ ID NO: 10172 represents the cDNA sequence for clone AI567858.

SEQ ID NO: 10173 represents the cDNA sequence for clone AI567860.

SEQ ID NO: 10174 represents the cDNA sequence for clone AI567861.

SEQ ID NO: 10175 represents the cDNA sequence for clone AI567863.

SEQ ID NO: 10176 represents the cDNA sequence for clone AI567868.

SEQ ID NO: 10177 represents the cDNA sequence for clone AI567869.

SEQ ID NO: 10178 represents the cDNA sequence for clone AI567871.

SEQ ID NO: 10179 represents the cDNA sequence for clone AI567873.

SEQ ID NO: 10180 represents the cDNA sequence for clone AI567935.

SEQ ID NO: 10181 represents the cDNA sequence for clone AI567939.

SEQ ID NO: 10182 represents the cDNA sequence for clone AI567941.

SEQ ID NO: 10183 represents the cDNA sequence for clone AI567943.

SEQ ID NO: 10184 represents the cDNA sequence for clone AI567945.

SEQ ID NO: 10185 represents the cDNA sequence for clone AI567947.

SEQ ID NO: 10186 represents the cDNA sequence for clone AI567948.

SEQ ID NO: 10187 represents the cDNA sequence for clone AI567958.

SEQ ID NO: 10188 represents the cDNA sequence for clone AI567960.

SEQ ID NO: 10189 represents the cDNA sequence for clone AI567961.

SEQ ID NO: 10190 represents the cDNA sequence for clone AI567967.

SEQ ID NO: 10191 represents the cDNA sequence for clone AI567976.

SEQ ID NO: 10192 represents the cDNA sequence for clone AI567979.

SEQ ID NO: 10193 represents the cDNA sequence for clone AI567983.

SEQ ID NO: 10194 represents the cDNA sequence for clone AI567983.

SEQ ID NO: 10195 represents the cDNA sequence for clone AI567986.

SEQ ID NO: 10196 represents the cDNA sequence for clone AI567988.

SEQ ID NO: 10197 represents the cDNA sequence for clone AI567984.

SEQ ID NO: 10198 represents the cDNA sequence for clone AI567994.

SEQ ID NO: 10199 represents the cDNA sequence for clone AI580229.

SEQ ID NO: 10200 represents the cDNA sequence for clone AI580232.

SEQ ID NO: 10201 represents the cDNA sequence for clone AI580237.

SEQ ID NO: 10202 represents the cDNA sequence for clone AI580237.

SEQ ID NO: 10203 represents the cDNA sequence for clone AI580238.

SEQ ID NO: 10204 represents the cDNA sequence for clone AI580244.

SEQ ID NO: 10205 represents the cDNA sequence for clone AI580248.

SEQ ID NO: 10206 represents the cDNA sequence for clone AI580249.

SEQ ID NO: 10207 represents the cDNA sequence for clone AI580252.

SEQ ID NO: 10208 represents the cDNA sequence for clone AI580254.

SEQ ID NO: 10209 represents the cDNA sequence for clone AI580261.

SEQ ID NO: 10210 represents the cDNA sequence for clone AI590577.

SEQ ID NO: 10211 represents the cDNA sequence for clone AI590578.

SEQ ID NO: 10212 represents the cDNA sequence for clone AI590580.

SEQ ID NO: 10213 represents the cDNA sequence for clone AI590584.

SEQ ID NO: 10214 represents the cDNA sequence for clone AI590592.

SEQ ID NO: 10215 represents the cDNA sequence for clone AI590593.

SEQ ID NO: 10216 represents the cDNA sequence for clone AI590596.

SEQ ID NO: 10217 represents the cDNA sequence for clone AI590599.

SEQ ID NO: 10218 represents the cDNA sequence for clone AI590600.

SEQ ID NO: 10219 represents the cDNA sequence for clone AI590602.

SEQ ID NO: 10220 represents the cDNA sequence for clone AI590604.

SEQ ID NO: 10221 represents the cDNA sequence for clone AI590605.

SEQ ID NO: 10222 represents the cDNA sequence for clone AI590606.

SEQ ID NO: 10223 represents the cDNA sequence for clone AI590610.

SEQ ID NO: 10224 represents the cDNA sequence for clone AI590613.

SEQ ID NO: 10225 represents the cDNA sequence for clone AI590615.

SEQ ID NO: 10226 represents the cDNA sequence for clone AI590616.

SEQ ID NO: 10227 represents the cDNA sequence for clone AI590617.

SEQ ID NO: 10228 represents the cDNA sequence for clone AI590618.

SEQ ID NO: 10229 represents the cDNA sequence for clone AI590621.

SEQ ID NO: 10230 represents the cDNA sequence for clone AI590623.

SEQ ID NO: 10231 represents the cDNA sequence for clone AI590629.

SEQ ID NO: 10232 represents the cDNA sequence for clone AI590631.

SEQ ID NO: 10233 represents the cDNA sequence for clone AI590636.

SEQ ID NO: 10234 represents the cDNA sequence for clone AI590642.

SEQ ID NO: 10235 represents the cDNA sequence for clone AI590643.

SEQ ID NO: 10236 represents the cDNA sequence for clone AI590646.

SEQ ID NO: 10237 represents the cDNA sequence for clone AI590648.

SEQ ID NO: 10238 represents the cDNA sequence for clone AI590649.

SEQ ID NO: 10239 represents the cDNA sequence for clone AI590757.

SEQ ID NO: 10240 represents the cDNA sequence for clone AI590759.

SEQ ID NO: 10241 represents the cDNA sequence for clone AI590760.

SEQ ID NO: 10242 represents the cDNA sequence for clone AI590765.

SEQ ID NO: 10243 represents the cDNA sequence for clone AI590766.

SEQ ID NO: 10244 represents the cDNA sequence for clone AI590770.

SEQ ID NO: 10245 represents the cDNA sequence for clone AI590771.

SEQ ID NO: 10246 represents the cDNA sequence for clone AI590772.

SEQ ID NO: 10247 represents the cDNA sequence for clone AI590781.

SEQ ID NO: 10248 represents the cDNA sequence for clone AI590784.

SEQ ID NO: 10249 represents the cDNA sequence for clone AI590786.

SEQ ID NO: 10250 represents the cDNA sequence for clone AI590788.

SEQ ID NO: 10251 represents the cDNA sequence for clone AI590790.

SEQ ID NO: 10252 represents the cDNA sequence for clone AI590791.

SEQ ID NO: 10253 represents the cDNA sequence for clone AI590795.

SEQ ID NO: 10254 represents the cDNA sequence for clone AI590798.

SEQ ID NO: 10255 represents the cDNA sequence for clone AI590799.

SEQ ID NO: 10256 represents the cDNA sequence for clone AI590800.

SEQ ID NO: 10257 represents the cDNA sequence for clone AI590804.

SEQ ID NO: 10258 represents the cDNA sequence for clone AI590806.

SEQ ID NO: 10259 represents the cDNA sequence for clone AI590809.

SEQ ID NO: 10260 represents the cDNA sequence for clone AI590887.

SEQ ID NO: 10261 represents the cDNA sequence for clone AI590888.

SEQ ID NO: 10262 represents the cDNA sequence for clone AI590890.

SEQ ID NO: 10263 represents the cDNA sequence for clone AI590892.

SEQ ID NO: 10264 represents the cDNA sequence for clone AI590893.

SEQ ID NO: 10265 represents the cDNA sequence for clone AI590895.

SEQ ID NO: 10266 represents the cDNA sequence for clone AI590899.

SEQ ID NO: 10267 represents the cDNA sequence for clone AI590903.

SEQ ID NO: 10268 represents the cDNA sequence for clone AI590904.

SEQ ID NO: 10269 represents the cDNA sequence for clone AI590905.

SEQ ID NO: 10270 represents the cDNA sequence for clone AI590914.

SEQ ID NO: 10271 represents the cDNA sequence for clone AI590997.

SEQ ID NO: 10272 represents the cDNA sequence for clone AI590998.

SEQ ID NO: 10273 represents the cDNA sequence for clone AI591001.

SEQ ID NO: 10274 represents the cDNA sequence for clone AI591003.

SEQ ID NO: 10275 represents the cDNA sequence for clone AI591004.

SEQ ID NO: 10276 represents the cDNA sequence for clone AI591005.

SEQ ID NO: 10277 represents the cDNA sequence for clone AI591007.

SEQ ID NO: 10278 represents the cDNA sequence for clone AI591008.

SEQ ID NO: 10279 represents the cDNA sequence for clone AI591009.

SEQ ID NO: 10280 represents the cDNA sequence for clone AI591011.

SEQ ID NO: 10281 represents the cDNA sequence for clone AI591016.

SEQ ID NO: 10282 represents the cDNA sequence for clone AI591018.

SEQ ID NO: 10283 represents the cDNA sequence for clone AI591021.

SEQ ID NO: 10284 represents the cDNA sequence for clone AI591022.

SEQ ID NO: 10285 represents the cDNA sequence for clone AI591027.

SEQ ID NO: 10286 represents the cDNA sequence for clone AI591029.

SEQ ID NO: 10287 represents the cDNA sequence for clone AI591032.

SEQ ID NO: 10288 represents the cDNA sequence for clone AI591033.

SEQ ID NO: 10289 represents the cDNA sequence for clone AI591035.

SEQ ID NO: 10290 represents the cDNA sequence for clone AI591038.

SEQ ID NO: 10291 represents the cDNA sequence for clone AI591046.

SEQ ID NO: 10292 represents the cDNA sequence for clone AI591049.

SEQ ID NO: 10293 represents the cDNA sequence for clone AI591050.

SEQ ID NO: 10294 represents the cDNA sequence for clone AI591051.

SEQ ID NO: 10295 represents the cDNA sequence for clone AI591053.

SEQ ID NO: 10296 represents the cDNA sequence for clone AI591055.

SEQ ID NO: 10297 represents the cDNA sequence for clone AI591056.

SEQ ID NO: 10298 represents the cDNA sequence for clone AI591060.

SEQ ID NO: 10299 represents the cDNA sequence for clone AI591064.

SEQ ID NO: 10300 represents the cDNA sequence for clone AI591065.

SEQ ID NO: 10301 represents the cDNA sequence for clone AI591068.

SEQ ID NO: 10302 represents the cDNA sequence for clone AI591070.

SEQ ID NO: 10303 represents the cDNA sequence for clone AI591072.

SEQ ID NO: 10304 represents the cDNA sequence for clone AI591117.

SEQ ID NO: 10305 represents the cDNA sequence for clone AI591118.

SEQ ID NO: 10306 represents the cDNA sequence for clone AI591121.

SEQ ID NO: 10307 represents the cDNA sequence for clone AI591127.

SEQ ID NO: 10308 represents the cDNA sequence for clone AI591128.

SEQ ID NO: 10309 represents the cDNA sequence for clone AI591129.

SEQ ID NO: 10310 represents the cDNA sequence for clone AI591131.

SEQ ID NO: 10311 represents the cDNA sequence for clone AI591134.

SEQ ID NO: 10312 represents the cDNA sequence for clone AI591135.

SEQ ID NO: 10313 represents the cDNA sequence for clone AI591136.

SEQ ID NO: 10314 represents the cDNA sequence for clone AI591140.

SEQ ID NO: 10315 represents the cDNA sequence for clone AI591141.

SEQ ID NO: 10316 represents the cDNA sequence for clone AI591143.

SEQ ID NO: 10317 represents the cDNA sequence for clone AI608688.

SEQ ID NO: 10318 represents the cDNA sequence for clone AI608690.

SEQ ID NO: 10319 represents the cDNA sequence for clone AI608695.

SEQ ID NO: 10320 represents the cDNA sequence for clone AI608696.

SEQ ID NO: 10321 represents the cDNA sequence for clone AI608698.

SEQ ID NO: 10322 represents the cDNA sequence for clone AI608699.

SEQ ID NO: 10323 represents the cDNA sequence for clone AI608700.

SEQ ID NO: 10324 represents the cDNA sequence for clone AI608701.

SEQ ID NO: 10325 represents the cDNA sequence for clone AI608704.

SEQ ID NO: 10326 represents the cDNA sequence for clone AI608708.

SEQ ID NO: 10327 represents the cDNA sequence for clone AI608709.

SEQ ID NO: 10328 represents the cDNA sequence for clone AI608710.

SEQ ID NO: 10329 represents the cDNA sequence for clone AI609057.

SEQ ID NO: 10330 represents the cDNA sequence for clone AI609060.

SEQ ID NO: 10331 represents the cDNA sequence for clone AI609064.

SEQ ID NO: 10332 represents the cDNA sequence for clone AI609067.

SEQ ID NO: 10333 represents the cDNA sequence for clone AI609070.

SEQ ID NO: 10334 represents the cDNA sequence for clone AI609073.

SEQ ID NO: 10335 represents the cDNA sequence for clone AI609075.

SEQ ID NO: 10336 represents the cDNA sequence for clone AI609076.

SEQ ID NO: 10337 represents the cDNA sequence for clone AI609324.

SEQ ID NO: 10338 represents the cDNA sequence for clone AI609325.

SEQ ID NO: 10339 represents the cDNA sequence for clone AI609327.

SEQ ID NO: 10340 represents the cDNA sequence for clone AI609329.

SEQ ID NO: 10341 represents the cDNA sequence for clone AI609334.

SEQ ID NO: 10342 represents the cDNA sequence for clone AI609335.

SEQ ID NO: 10343 represents the cDNA sequence for clone AI609337.

SEQ ID NO: 10344 represents the cDNA sequence for clone AI609338.

SEQ ID NO: 10345 represents the cDNA sequence for clone AI609342.

SEQ ID NO: 10346 represents the cDNA sequence for clone AI609343.

SEQ ID NO: 10347 represents the cDNA sequence for clone AI609345.

SEQ ID NO: 10348 represents the cDNA sequence for clone AI609346.

SEQ ID NO: 10349 represents the cDNA sequence for clone AI609349.

SEQ ID NO: 10350 represents the cDNA sequence for clone AI609351.

SEQ ID NO: 10351 represents the cDNA sequence for clone AI609352.

SEQ ID NO: 10352 represents the cDNA sequence for clone AI609353.

SEQ ID NO: 10353 represents the cDNA sequence for clone AI609355.

SEQ ID NO: 10354 represents the cDNA sequence for clone AI609356.

SEQ ID NO: 10355 represents the cDNA sequence for clone AI609359.

SEQ ID NO: 10356 represents the cDNA sequence for clone AI609361.

SEQ ID NO: 10357 represents the cDNA sequence for clone AI609367.

SEQ ID NO: 10358 represents the cDNA sequence for clone AI609369.

SEQ ID NO: 10359 represents the cDNA sequence for clone AI609370.

SEQ ID NO: 10360 represents the cDNA sequence for clone AI609373.

SEQ ID NO: 10361 represents the cDNA sequence for clone AI609374.

SEQ ID NO: 10362 represents the cDNA sequence for clone AI609377.

SEQ ID NO: 10363 represents the cDNA sequence for clone AI609573.

SEQ ID NO: 10364 represents the cDNA sequence for clone AI609576.

SEQ ID NO: 10365 represents the cDNA sequence for clone AI609578.

SEQ ID NO: 10366 represents the cDNA sequence for clone AI609587.

SEQ ID NO: 10367 represents the cDNA sequence for clone AI609590.

SEQ ID NO: 10368 represents the cDNA sequence for clone AI609591.

SEQ ID NO: 10369 represents the cDNA sequence for clone AI609595.

SEQ ID NO: 10370 represents the cDNA sequence for clone AI609667.

SEQ ID NO: 10371 represents the cDNA sequence for clone AI609669.

SEQ ID NO: 10372 represents the cDNA sequence for clone AI609676.

SEQ ID NO: 10373 represents the cDNA sequence for clone AI609681.

SEQ ID NO: 10374 represents the cDNA sequence for clone AI609682.

SEQ ID NO: 10375 represents the cDNA sequence for clone AI609683.

SEQ ID NO: 10376 represents the cDNA sequence for clone AI609689.

SEQ ID NO: 10377 represents the cDNA sequence for clone AI612817.

SEQ ID NO: 10378 represents the cDNA sequence for clone AI612820.

SEQ ID NO: 10379 represents the cDNA sequence for clone AI612822.

SEQ ID NO: 10380 represents the cDNA sequence for clone AI612825.

SEQ ID NO: 10381 represents the cDNA sequence for clone AI612826.

SEQ ID NO: 10382 represents the cDNA sequence for clone AI612828.

SEQ ID NO: 10383 represents the cDNA sequence for clone AI612833.

SEQ ID NO: 10384 represents the cDNA sequence for clone AI612834.

SEQ ID NO: 10385 represents the cDNA sequence for clone AI612842.

SEQ ID NO: 10386 represents the cDNA sequence for clone AI612844.

SEQ ID NO: 10387 represents the cDNA sequence for clone AI612846.

SEQ ID NO: 10388 represents the cDNA sequence for clone AI612849.

SEQ ID NO: 10389 represents the cDNA sequence for clone AI612850.

SEQ ID NO: 10390 represents the cDNA sequence for clone AI612851.

SEQ ID NO: 10391 represents the cDNA sequence for clone AI612853.

SEQ ID NO: 10392 represents the cDNA sequence for clone AI612854.

SEQ ID NO: 10393 represents the cDNA sequence for clone AI612857.

SEQ ID NO: 10394 represents the cDNA sequence for clone AI612867.

SEQ ID NO: 10395 represents the cDNA sequence for clone AI633439.

SEQ ID NO: 10396 represents the cDNA sequence for clone AI633442.

SEQ ID NO: 10397 represents the cDNA sequence for clone AI633468.

SEQ ID NO: 10398 represents the cDNA sequence for clone AI633474.

SEQ ID NO: 10399 represents the cDNA sequence for clone AI633475.

SEQ ID NO: 10400 represents the cDNA sequence for clone AI633478.

SEQ ID NO: 10401 represents the cDNA sequence for clone AI633481.

SEQ ID NO: 10402 represents the cDNA sequence for clone AI633494.

SEQ ID NO: 10403 represents the cDNA sequence for clone AI633495.

SEQ ID NO: 10404 represents the cDNA sequence for clone AI648453.

SEQ ID NO: 10405 represents the cDNA sequence for clone AI648459.

SEQ ID NO: 10406 represents the cDNA sequence for clone AI648461.

SEQ ID NO: 10407 represents the cDNA sequence for clone AI648462.

SEQ ID NO: 10408 represents the cDNA sequence for clone AI648465.

SEQ ID NO: 10409 represents the cDNA sequence for clone AI648468.

SEQ ID NO: 10410 represents the cDNA sequence for clone AI648470.

SEQ ID NO: 10411 represents the cDNA sequence for clone AI648471.

SEQ ID NO: 10412 represents the cDNA sequence for clone AI648481.

SEQ ID NO: 10413 represents the cDNA sequence for clone AI648483.

SEQ ID NO: 10414 represents the cDNA sequence for clone AI648488.

SEQ ID NO: 10415 represents the cDNA sequence for clone AI648489.

SEQ ID NO: 10416 represents the cDNA sequence for clone AI648495.

SEQ ID NO: 10417 represents the cDNA sequence for clone AI648497.

SEQ ID NO: 10418 represents the cDNA sequence for clone AI648501.

SEQ ID NO: 10419 represents the cDNA sequence for clone AI648504.

SEQ ID NO: 10420 represents the cDNA sequence for clone AI648505.

SEQ ID NO: 10421 represents the cDNA sequence for clone AI648506.

SEQ ID NO: 10422 represents the cDNA sequence for clone AI648510.

SEQ ID NO: 10423 represents the cDNA sequence for clone AI648511.

SEQ ID NO: 10424 represents the cDNA sequence for clone AI648512.

SEQ ID NO: 10425 represents the cDNA sequence for clone AI648513.

SEQ ID NO: 10426 represents the cDNA sequence for clone AI648518.

SEQ ID NO: 10427 represents the cDNA sequence for clone AI648519.

SEQ ID NO: 10428 represents the cDNA sequence for clone AI648527.

SEQ ID NO: 10429 represents the cDNA sequence for clone AI648530.

SEQ ID NO: 10430 represents the cDNA sequence for clone AI648532.

SEQ ID NO: 10431 represents the cDNA sequence for clone AI648533.

SEQ ID NO: 10432 represents the cDNA sequence for clone AI648534.

SEQ ID NO: 10433 represents the cDNA sequence for clone AI648535.

SEQ ID NO: 10434 represents the cDNA sequence for clone AI648536.

SEQ ID NO: 10435 represents the cDNA sequence for clone AI648545.

SEQ ID NO: 10436 represents the cDNA sequence for clone AI648546.

SEQ ID NO: 10437 represents the cDNA sequence for clone AI648548.

SEQ ID NO: 10438 represents the cDNA sequence for clone AI648550.

SEQ ID NO: 10439 represents the cDNA sequence for clone AI648552.

SEQ ID NO: 10440 represents the cDNA sequence for clone AI648554.

SEQ ID NO: 10441 represents the cDNA sequence for clone AI648558.

SEQ ID NO: 10442 represents the cDNA sequence for clone AI648563.

SEQ ID NO: 10443 represents the cDNA sequence for clone AI648564.

SEQ ID NO: 10444 represents the cDNA sequence for clone AI648565.

SEQ ID NO: 10445 represents the cDNA sequence for clone AI648566.

SEQ ID NO: 10446 represents the cDNA sequence for clone AI648576.

SEQ ID NO: 10447 represents the cDNA sequence for clone AI648578.

SEQ ID NO: 10448 represents the cDNA sequence for clone AI648579.

SEQ ID NO: 10449 represents the cDNA sequence for clone AI648580.

SEQ ID NO: 10450 represents the cDNA sequence for clone AI648584.

SEQ ID NO: 10451 represents the cDNA sequence for clone AI648585.

SEQ ID NO: 10452 represents the cDNA sequence for clone AI648586.

SEQ ID NO: 10453 represents the cDNA sequence for clone AI648588.

SEQ ID NO: 10454 represents the cDNA sequence for clone AI648593.

SEQ ID NO: 10455 represents the cDNA sequence for clone AI648596.

SEQ ID NO: 10456 represents the cDNA sequence for clone AI648600.

SEQ ID NO: 10457 represents the cDNA sequence for clone AI653439.

SEQ ID NO: 10458 represents the cDNA sequence for clone AI653445.

SEQ ID NO: 10459 represents the cDNA sequence for clone AI653457.

SEQ ID NO: 10460 represents the cDNA sequence for clone AI653459.

SEQ ID NO: 10461 represents the cDNA sequence for clone AI653460.

SEQ ID NO: 10462 represents the cDNA sequence for clone AI653464.

SEQ ID NO: 10463 represents the cDNA sequence for clone AI653472.

SEQ ID NO: 10464 represents the cDNA sequence for clone AI653482.

SEQ ID NO: 10465 represents the cDNA sequence for clone AI653487.

SEQ ID NO: 10466 represents the cDNA sequence for clone AI653489.

SEQ ID NO: 10467 represents the cDNA sequence for clone AI653490.

SEQ ID NO; 10468 represents the cDNA sequence for clone AI653493.

SEQ ID NO: 10469 represents the cDNA sequence for clone AI653499.

SEQ ID NO: 10470 represents the cDNA sequence for clone AI653502.

SEQ ID NO: 10471 represents the cDNA sequence for clone AI653506.

SEQ ID NO: 10472 represents the cDNA sequence for clone AI653508.

SEQ ID NO: 10473 represents the cDNA sequence for clone AI653511.

SEQ ID NO: 10474 represents the cDNA sequence for clone AI653512.

SEQ ID NO: 10475 represents the cDNA sequence for clone AI653520.

SEQ ID NO: 10476 represents the cDNA sequence for clone AI653524.

SEQ ID NO: 10477 represents the cDNA sequence for clone AI653533.

SEQ ID NO: 10478 represents the cDNA sequence for clone AI653538.

SEQ ID NO: 10479 represents the cDNA sequence for clone AI653547.

SEQ ID NO: 10480 represents the cDNA sequence for clone AI653551.

SEQ ID NO: 10481 represents the cDNA sequence for clone AI653555.

SEQ ID NO: 10482 represents the cDNA sequence for clone AI653558.

SEQ ID NO: 10483 represents the cDNA sequence for clone AI653559.

SEQ ID NO: 10484 represents the cDNA sequence for clone AI653564.

SEQ ID NO: 10485 represents the cDNA sequence for clone AI653567.

SEQ ID NO: 10486 represents the cDNA sequence for clone AI653594.

SEQ ID NO: 10487 represents the cDNA sequence for clone AI654241.

SEQ ID NO: 10488 represents the cDNA sequence for clone AI654242.

SEQ ID NO: 10489 represents the cDNA sequence for clone AI654245.

SEQ ID NO: 10490 represents the cDNA sequence for clone AI654251.

SEQ ID NO: 10491 represents the cDNA sequence for clone AI654256.

SEQ ID NO: 10492 represents the cDNA sequence for clone AI654257.

SEQ ID NO: 10493 represents the cDNA sequence for clone AI654258.

SEQ ID NO: 10494 represents the cDNA sequence for clone AI654261.

SEQ ID NO: 10495 represents the cDNA sequence for clone AI654268.

SEQ ID NO: 10496 represents the cDNA sequence for clone AI654291.

SEQ ID NO: 10497 represents the cDNA sequence for clone AI654292.

SEQ ID NO: 10498 represents the cDNA sequence for clone AI654298.

SEQ ID NO: 10499 represents the cDNA sequence for clone AI654299.

SEQ ID NO: 10500 represents the cDNA sequence for clone AI654300.

SEQ ID NO: 10501 represents the cDNA sequence for clone AI654306.

SEQ ID NO: 10502 represents the cDNA sequence for clone AI654313.

SEQ ID NO: 10503 represents the cDNA sequence for clone AI654316.

SEQ ID NO: 10504 represents the cDNA sequence for clone AI654321.

SEQ ID NO: 10505 represents the cDNA sequence for clone AI654322.

SEQ ID NO: 10506 represents the cDNA sequence for clone AI654383.

SEQ ID NO: 10507 represents the cDNA sequence for clone AI654387.

SEQ ID NO: 10508 represents the cDNA sequence for clone AI654400.

SEQ ID NO: 10509 represents the cDNA sequence for clone AI654403.

SEQ ID NO: 10510 represents the cDNA sequence for clone AI654450.

SEQ ID NO: 10511 represents the cDNA sequence for clone AI654452.

SEQ ID NO: 10512 represents the cDNA sequence for clone AI654453.

SEQ ID NO: 10513 represents the cDNA sequence for clone AI654454.

SEQ ID NO: 10514 represents the cDNA sequence for clone AI654459.

SEQ ID NO: 10515 represents the cDNA sequence for clone AI654462.

SEQ ID NO: 10516 represents the cDNA sequence for clone AI654470.

SEQ ID NO: 10517 represents the cDNA sequence for clone AI654471.

SEQ ID NO: 10518 represents the cDNA sequence for clone AI654472.

SEQ ID NO: 10519 represents the cDNA sequence for clone AI654477.

SEQ ID NO: 10520 represents the cDNA sequence for clone AI654478.

SEQ ID NO: 10521 represents the cDNA sequence for clone AI654517.

SEQ ID NO: 10522 represents the cDNA sequence for clone AI654518.

SEQ ID NO: 10523 represents the cDNA sequence for clone AI654519.

SEQ ID NO: 10524 represents the cDNA sequence for clone AI654522.

SEQ ID NO: 10525 represents the cDNA sequence for clone AI654529.

SEQ ID NO: 10526 represents the cDNA sequence for clone AI654530.

SEQ ID NO: 10527 represents the cDNA sequence for clone AI654533.

SEQ ID NO: 10528 represents the cDNA sequence for clone AI654592.

SEQ ID NO: 10529 represents the cDNA sequence for clone AI654600.

SEQ ID NO: 10530 represents the cDNA sequence for clone AI654652.

SEQ ID NO: 10531 represents the cDNA sequence for clone AI654654.

SEQ ID NO: 10532 represents the cDNA sequence for clone AI654659.

SEQ ID NO: 10533 represents the cDNA sequence for clone AI654660.

SEQ ID NO: 10534 represents the cDNA sequence for clone AI654663.

SEQ ID NO: 10535 represents the cDNA sequence for clone AI654671.

SEQ ID NO: 10536 represents the cDNA sequence for clone AI654673.

SEQ ID NO: 10537 represents the cDNA sequence for clone AI654674.

SEQ ID NO: 10538 represents the cDNA sequence for clone AI654675.

SEQ ID NO: 10539 represents the cDNA sequence for clone AI654677.

SEQ ID NO: 10540 represents the cDNA sequence for clone AI654678.

SEQ ID NO: 10541 represents the cDNA sequence for clone AI654734.

SEQ ID NO: 10542 represents the cDNA sequence for clone AI654737.

SEQ ID NO: 10543 represents the cDNA sequence for clone AI654738.

SEQ ID NO: 10544 represents the cDNA sequence for clone AI654739.

SEQ ID NO: 10545 represents the cDNA sequence for clone AI654740.

SEQ ID NO: 10546 represents the cDNA sequence for clone AI654744.

SEQ ID NO: 10547 represents the cDNA sequence for clone AI654745.

SEQ ID NO: 10548 represents the cDNA sequence for clone AI654748.

SEQ ID NO: 10549 represents the cDNA sequence for clone AI654749.

SEQ ID NO: 10550 represents the cDNA sequence for clone AI654752.

SEQ ID NO: 10551 represents the cDNA sequence for clone AI654757.

SEQ ID NO: 10552 represents the cDNA sequence for clone AI654758.

SEQ ID NO: 10553 represents the cDNA sequence for clone AI672105.

SEQ ID NO: 10554 represents the cDNA sequence for clone AI672108.

SEQ ID NO: 10555 represents the cDNA sequence for clone AI672110.

SEQ ID NO: 10556 represents the cDNA sequence for clone AI672115.

SEQ ID NO: 10557 represents the cDNA sequence for clone AI672117.

SEQ ID NO: 10558 represents the cDNA sequence for clone AI672119.

SEQ ID NO: 10559 represents the cDNA sequence for clone AI672122.

SEQ ID NO: 10560 represents the cDNA sequence for clone AI672123.

SEQ ID NO: 10561 represents the cDNA sequence for clone AI672130.

SEQ ID NO: 10562 represents the cDNA sequence for clone AF126507.

SEQ ID NO: 10563 represents the cDNA sequence for clone AF136410.

SEQ ID NO: 10564 represents the cDNA sequence for clone AF136412.

SEQ ID NO: 10565 represents the cDNA sequence for clone AF136413.

SEQ ID NO: 10566 represents the cDNA sequence for clone AF136414.

SEQ ID NO: 10567 represents the cDNA sequence for clone AF136415.

SEQ ID NO: 10568 represents the cDNA sequence for clone AF136416.

SEQ ID NO: 10569 represents the cDNA sequence for clone AF136417.

SEQ ID NO: 10570 represents the cDNA sequence for clone AI732121.

SEQ ID NO: 10571 represents the cDNA sequence for clone AI732179.

SEQ ID NO: 10572 represents the cDNA sequence for clone AI732347.

SEQ ID NO: 10573 represents the cDNA sequence for clone AI732348.

SEQ ID NO: 10574 represents the cDNA sequence for clone AI732349.

SEQ ID NO: 10575 represents the cDNA sequence for clone AI732379.

SEQ ID NO: 10576 represents the cDNA sequence for clone AI732495.

SEQ ID NO: 10577 represents the cDNA sequence for clone AI732496.

SEQ ID NO: 10578 represents the cDNA sequence for clone AI732497.

SEQ ID NO: 10579 represents the cDNA sequence for clone AI732498.

SEQ ID NO: 10580 represents the cDNA sequence for clone AI732499.

SEQ ID NO: 10581 represents the cDNA sequence for clone AI732500.

SEQ ID NO: 10582 represents the cDNA sequence for clone AI732501.

SEQ ID NO: 10583 represents the cDNA sequence for clone AI732502.

SEQ ID NO: 10584 represents the cDNA sequence for clone AI732543.

SEQ ID NO: 10585 represents the cDNA sequence for clone AI732544.

SEQ ID NO: 10586 represents the cDNA sequence for clone AI732545.

SEQ ID NO: 10587 represents the cDNA sequence for clone AI732575.

SEQ ID NO: 10588 represents the cDNA sequence for clone AI732576.

SEQ ID NO: 10589 represents the cDNA sequence for clone AI732577.

SEQ ID NO: 10590 represents the cDNA sequence for clone AI732578.

SEQ ID NO: 10591 represents the cDNA sequence for clone AI732579.

SEQ ID NO: 10592 represents the cDNA sequence for clone AI732580.

SEQ ID NO: 10593 represents the cDNA sequence for clone AI732581.

SEQ ID NO: 10594 represents the cDNA sequence for clone AI732582.

SEQ ID NO: 10595 represents the cDNA sequence for clone AI732583.

SEQ ID NO: 10596 represents the cDNA sequence for clone AI732584.

SEQ ID NO: 10597 represents the cDNA sequence for clone AI732585.

SEQ ID NO: 10598 represents the cDNA sequence for clone AI732586.

SEQ ID NO: 10599 represents the cDNA sequence for clone AI732587.

SEQ ID NO: 10600 represents the cDNA sequence for clone AI732609.

SEQ ID NO: 10601 represents the cDNA sequence for clone AI732610.

SEQ ID NO: 10602 represents the cDNA sequence for clone AI732611.

SEQ ID NO: 10603 represents the cDNA sequence for clone AI732612.

SEQ ID NO: 10604 represents the cDNA sequence for clone AI732613.

SEQ ID NO: 10605 represents the cDNA sequence for clone AI732614.

SEQ ID NO: 10606 represents the cDNA sequence for clone AI732615.

SEQ ID NO: 10607 represents the cDNA sequence for clone AI732616.

SEQ ID NO: 10608 represents the cDNA sequence for clone AI732617.

SEQ ID NO: 10609 represents the cDNA sequence for clone AI732618.

SEQ ID NO: 10610 represents the cDNA sequence for clone AI732619.

SEQ ID NO: 10611 represents the cDNA sequence for clone AI732620.

SEQ ID NO: 10612 represents the cDNA sequence for clone AI732621.

SEQ ID NO: 10613 represents the cDNA sequence for clone AI732622.

SEQ ID NO: 10614 represents the cDNA sequence for clone AI732623.

SEQ ID NO: 10615 represents the cDNA sequence for clone AI732624.

SEQ ID NO: 10616 represents the cDNA sequence for clone AI732625.

SEQ ID NO: 10617 represents the cDNA sequence for clone AI732692.

SEQ ID NO: 10618 represents the cDNA sequence for clone AI732693.

SEQ ID NO: 10619 represents the cDNA sequence for clone AI732694.

SEQ ID NO: 10620 represents the cDNA sequence for clone AI732695.

SEQ ID NO: 10621 represents the cDNA sequence for clone AI732697.

SEQ ID NO: 10622 represents the cDNA sequence for clone AI732698.

SEQ ID NO: 10623 represents the cDNA sequence for clone AI732699.

SEQ ID NO: 10624 represents the cDNA sequence for clone AI732700.

SEQ ID NO: 10625 represents the cDNA sequence for clone AI732702.

SEQ ID NO: 10626 represents the cDNA sequence for clone AI732705.

SEQ ID NO: 10627 represents the cDNA sequence for clone AI732706.

SEQ ID NO: 10628 represents the cDNA sequence for clone AI732727.

SEQ ID NO: 10629 represents the cDNA sequence for clone AI732728.

SEQ ID NO: 10630 represents the cDNA sequence for clone AI732729.

SEQ ID NO: 10631 represents the cDNA sequence for clone AI732730.

SEQ ID NO: 10632 represents the cDNA sequence for clone AI732731.

SEQ ID NO: 10633 represents the cDNA sequence for clone AI732732.

SEQ ID NO: 10634 represents the cDNA sequence for clone AI732733.

SEQ ID NO: 10635 represents the cDNA sequence for clone AI732734.

SEQ ID NO: 10636 represents the cDNA sequence for clone AI732736.

SEQ ID NO: 10637 represents the cDNA sequence for clone AI732737.

SEQ ID NO: 10638 represents the cDNA sequence for clone AI732738.

SEQ ID NO: 10639 represents the cDNA sequence for clone AI732739.

SEQ ID NO: 10640 represents the cDNA sequence for clone AI732740.

SEQ ID NO: 10641 represents the cDNA sequence for clone AI732741.

SEQ ID NO: 10642 represents the cDNA sequence for clone AI732742.

SEQ ID NO: 10643 represents the cDNA sequence for clone AI732744.

SEQ ID NO: 10644 represents the cDNA sequence for clone AI732746.

SEQ ID NO: 10645 represents the cDNA sequence for clone AI732747.

SEQ ID NO: 10646 represents the cDNA sequence for clone AI732749.

SEQ ID NO: 10647 represents the cDNA sequence for clone AI732763.

SEQ ID NO: 10648 represents the cDNA sequence for clone AI732764.

SEQ ID NO: 10649 represents the cDNA sequence for clone AI732765.

SEQ ID NO: 10650 represents the cDNA sequence for clone AI732766.

SEQ ID NO: 10651 represents the cDNA sequence for clone AI732767.

SEQ ID NO: 10652 represents the cDNA sequence for clone AI732768.

SEQ ID NO: 10653 represents the cDNA sequence for clone AI732769.

SEQ ID NO: 10654 represents the cDNA sequence for clone AI732770.

SEQ ID NO: 10655 represents the cDNA sequence for clone AI732773.

SEQ ID NO: 10656 represents the cDNA sequence for clone AI732774.

SEQ ID NO: 10657 represents the cDNA sequence for clone AI732776.

SEQ ID NO: 10658 represents the cDNA sequence for clone AI732777.

SEQ ID NO: 10659 represents the cDNA sequence for clone AI732778.

SEQ ID NO: 10660 represents the cDNA sequence for clone AI732779.

SEQ ID NO: 10661 represents the cDNA sequence for clone AI732780.

SEQ ID NO: 10662 represents the cDNA sequence for clone AI732781.

SEQ ID NO: 10663 represents the cDNA sequence for clone AI732782.

SEQ ID NO: 10664 represents the cDNA sequence for clone AI732783.

SEQ ID NO: 10665 represents the cDNA sequence for clone AI732784.

SEQ ID NO: 10666 represents the cDNA sequence for clone AI732807.

SEQ ID NO: 10667 represents the cDNA sequence for clone AI732808.

SEQ ID NO: 10668 represents the cDNA sequence for clone AI732810.

SEQ ID NO: 10669 represents the cDNA sequence for clone AI732812.

SEQ ID NO: 10670 represents the cDNA sequence for clone AI732813.

SEQ ID NO: 10671 represents the cDNA sequence for clone AI732814.

SEQ ID NO: 10672 represents the cDNA sequence for clone AI732816.

SEQ ID NO: 10673 represents the cDNA sequence for clone AI732817.

SEQ ID NO: 10674 represents the cDNA sequence for clone AI732819.

SEQ ID NO: 10675 represents the cDNA sequence for clone AI732820.

SEQ ID NO: 10676 represents the cDNA sequence for clone AI732821.

SEQ ID NO: 10677 represents the cDNA sequence for clone AI732823.

SEQ ID NO: 10678 represents the cDNA sequence for clone AI732824.

SEQ ID NO: 10679 represents the cDNA sequence for clone AI732825.

SEQ ID NO: 10680 represents the cDNA sequence for clone AI732826.

SEQ ID NO: 10681 represents the cDNA sequence for clone AI732827.

SEQ ID NO: 10682 represents the cDNA sequence for clone AI732828.

SEQ ID NO: 10683 represents the cDNA sequence for clone AI732856.

SEQ ID NO: 10684 represents the cDNA sequence for clone AI732858

SEQ ID NO: 10685 represents the cDNA sequence for clone AI732859.

SEQ ID NO: 10686 represents the cDNA sequence for clone AI732861.

SEQ ID NO: 10687 represents the cDNA sequence for clone AI732862.

SEQ ID NO: 10688 represents the cDNA sequence for clone AI732863.

SEQ ID NO: 10689 represents the cDNA sequence for clone AI732864.

SEQ ID NO: 10690 represents the cDNA sequence for clone AI732865.

SEQ ID NO: 10691 represents the cDNA sequence for clone AI732866.

SEQ ID NO: 10692 represents the cDNA sequence for clone AI732867.

SEQ ID NO: 10693 represents the cDNA sequence for clone AI732868.

SEQ ID NO: 10694 represents the cDNA sequence for clone AI732869.

SEQ ID NO: 10695 represents the cDNA sequence for clone AI732908.

SEQ ID NO: 10696 represents the cDNA sequence for clone AI732910.

SEQ ID NO: 10697 represents the cDNA sequence for clone AI733712.

SEQ ID NO: 10698 represents the cDNA sequence for clone AI733713.

SEQ ID NO: 10699 represents the cDNA sequence for clone AI733716.

SEQ ID NO: 10700 represents the cDNA sequence for clone AI733871.

SEQ ID NO: 10701 represents the cDNA sequence for clone AI733872.

SEQ ID NO: 10702 represents the cDNA sequence for clone AI733873.

SEQ ID NO: 10703 represents the cDNA sequence for clone AI733874.

SEQ ID NO: 10704 represents the cDNA sequence for clone AI733875.

SEQ ID NO: 10705 represents the cDNA sequence for clone AI733876.

SEQ ID NO: 10706 represents the cDNA sequence for clone AI733877.

SEQ ID NO: 10707 represents the cDNA sequence for clone AI733878.

SEQ ID NO: 10708 represents the cDNA sequence for clone AI733879.

SEQ ID NO: 10709 represents the cDNA sequence for clone AI733880.

SEQ ID NO: 10710 represents the cDNA sequence for clone AI733881.

SEQ ID NO: 10711 represents the cDNA sequence for clone AI733883.

SEQ ID NO: 10712 represents the cDNA sequence for clone AI733902.

SEQ ID NO: 10713 represents the cDNA sequence for clone AI733903.

SEQ ID NO: 10714 represents the cDNA sequence for clone AI733904.

SEQ ID NO: 10715 represents the cDNA sequence for clone AI733905.

SEQ ID NO: 10716 represents the cDNA sequence for clone AI733906.

SEQ ID NO: 10717 represents the cDNA sequence for clone AI733907.

SEQ ID NO: 10718 represents the cDNA sequence for clone AI733908.

SEQ ID NO: 10719 represents the cDNA sequence for clone AI733909

SEQ ID NO: 10720 represents the cDNA sequence for clone AI733910.

SEQ ID NO: 10721 represents the cDNA sequence for clone AI733911.

SEQ ID NO: 10722 represents the cDNA sequence for clone AI733912.

SEQ ID NO: 10723 represents the cDNA sequence for clone AI733914.

SEQ ID NO: 10724 represents the cDNA sequence for clone AI733915.

SEQ ID NO: 10725 represents the cDNA sequence for clone AI733916.

SEQ ID NO: 10726 represents the cDNA sequence for clone AI733917.

SEQ ID NO: 10727 represents the cDNA sequence for clone AI733918.

SEQ ID NO: 10728 represents the cDNA sequence for clone AI733919.

SEQ ID NO: 10729 represents the cDNA sequence for clone AI733921.

SEQ ID NO: 10730 represents the cDNA sequence for clone AI733922.

SEQ ID NO: 10731 represents the cDNA sequence for clone AI733923.

SEQ ID NO: 10732 represents the cDNA sequence for clone AI733926.

SEQ ID NO: 10733 represents the cDNA sequence for clone AI733927.

SEQ ID NO: 10734 represents the cDNA sequence for clone AI733928.

SEQ ID NO: 10735 represents the cDNA sequence for clone AI733930.

SEQ ID NO: 10736 represents the cDNA sequence for clone AI733931.

SEQ ID NO: 10737 represents the cDNA sequence for clone AI733932.

SEQ ID NO: 10738 represents the cDNA sequence for clone AI733933.

SEQ ID NO: 10739 represents the cDNA sequence for clone AI733934.

SEQ ID NO: 10740 represents the cDNA sequence for clone AI733935.

SEQ ID NO: 10741 represents the cDNA sequence for clone AI733936.

SEQ ID NO: 10742 represents the cDNA sequence for clone AI733937.

SEQ ID NO: 10743 represents the cDNA sequence for clone AI733938.

SEQ ID NO: 10744 represents the cDNA sequence for clone AI733939.

SEQ ID NO: 10745 represents the cDNA sequence for clone AI733941.

SEQ ID NO: 10746 represents the cDNA sequence for clone AI733942.

SEQ ID NO: 10747 represents the cDNA sequence for clone AI733943.

SEQ ID NO: 10748 represents the cDNA sequence for clone AI733944.

SEQ ID NO: 10749 represents the cDNA sequence for clone AI733946.

SEQ ID NO: 10750 represents the cDNA sequence for clone AI733947.

SEQ ID NO: 10751 represents the cDNA sequence for clone AI733950.

SEQ ID NO: 10752 represents the cDNA sequence for clone AI733951.

SEQ ID NO: 10753 represents the cDNA sequence for clone AI733952.

SEQ ID NO: 10754 represents the cDNA sequence for clone AI733953.

SEQ ID NO: 10755 represents the cDNA sequence for clone AI733954.

SEQ ID NO: 10756 represents the cDNA sequence for clone AI733955.

SEQ ID NO: 10757 represents the cDNA sequence for clone AI733957.

SEQ ID NO: 10758 represents the cDNA sequence for clone AI733958.

SEQ ID NO: 10759 represents the cDNA sequence for clone AI733959.

SEQ ID NO: 10760 represents the cDNA sequence for clone AI733960.

SEQ ID NO: 10761 represents the cDNA sequence for clone AI733961.

SEQ ID NO: 10762 represents the cDNA sequence for clone AI733962.

SEQ ID NO: 10763 represents the cDNA sequence for clone AI733964.

SEQ ID NO: 10764 represents the cDNA sequence for clone AI733966.

SEQ ID NO: 10765 represents the cDNA sequence for clone AI733976.

SEQ ID NO: 10766 represents the cDNA sequence for clone AI733977.

SEQ ID NO: 10767 represents the cDNA sequence for clone AI733979.

SEQ ID NO: 10768 represents the cDNA sequence for clone AI733980.

SEQ ID NO: 10769 represents the cDNA sequence for clone AI733981.

SEQ ID NO: 10770 represents the cDNA sequence for clone AI733983.

SEQ ID NO: 10771 represents the cDNA sequence for clone AI733984.

SEQ ID NO: 10772 represents the cDNA sequence for clone AI733986.

SEQ ID NO: 10773 represents the cDNA sequence for clone AI733988.

SEQ ID NO: 10774 represents the cDNA sequence for clone AI733989.

SEQ ID NO: 10775 represents the cDNA sequence for clone AI733990.

SEQ ID NO: 10776 represents the cDNA sequence for clone AI733991.

SEQ ID NO: 10777 represents the cDNA sequence for clone AI733992.

SEQ ID NO: 10778 represents the cDNA sequence for clone AI733994.

SEQ ID NO: 10779 represents the cDNA sequence for clone AI733995.

SEQ ID NO: 10780 represents the cDNA sequence for clone AI733997.

SEQ ID NO: 10781 represents the cDNA sequence for clone AI733998.

SEQ ID NO: 10782 represents the cDNA sequence for clone AI734001.

SEQ ID NO: 10783 represents the cDNA sequence for clone AI734002.

SEQ ID NO: 10784 represents the cDNA sequence for clone AI734003.

SEQ ID NO: 10785 represents the cDNA sequence for clone AI734018.

SEQ ID NO: 10786 represents the cDNA sequence for clone AI734020.

SEQ ID NO: 10787 represents the cDNA sequence for clone AI734021.

SEQ ID NO: 10788 represents the cDNA sequence for clone AI734022.

SEQ ID NO: 10789 represents the cDNA sequence for clone AI734023.

SEQ ID NO: 10790 represents the cDNA sequence for clone AI734025.

SEQ ID NO: 10791 represents the cDNA sequence for clone AI734026.

SEQ ID NO: 10792 represents the cDNA sequence for clone AI734027.

SEQ ID NO: 10793 represents the cDNA sequence for clone AI734028.

SEQ ID NO: 10794 represents the cDNA sequence for clone AI734030.

SEQ ID NO: 10795 represents the cDNA sequence for clone AI734031.

SEQ ID NO: 10796 represents the cDNA sequence for clone AI734032.

SEQ ID NO: 10797 represents the cDNA sequence for clone AI734033.

SEQ ID NO: 10798 represents the cDNA sequence for clone AI734034.

SEQ ID NO: 10799 represents the cDNA sequence for clone AI734037.

SEQ ID NO: 10800 represents the cDNA sequence for clone AI734038.

SEQ ID NO: 10801 represents the cDNA sequence for clone AI734039.

SEQ ID NO: 10802 represents the cDNA sequence for clone AI734040.

SEQ ID NO: 10803 represents the cDNA sequence for clone AI734041.

SEQ ID NO: 10804 represents the cDNA sequence for clone AI734042.

SEQ ID NO: 10805 represents the cDNA sequence for clone AI734043.

SEQ ID NO: 10806 represents the cDNA sequence for clone AI734045.

SEQ ID NO: 10807 represents the cDNA sequence for clone AI734046.

SEQ ID NO: 10808 represents the cDNA sequence for clone AI734047.

SEQ ID NO: 10809 represents the cDNA sequence for clone AI734048.

SEQ ID NO: 10810 represents the cDNA sequence for clone AI734099.

SEQ ID NO: 10811 represents the cDNA sequence for clone AI734100.

SEQ ID NO: 10812 represents the cDNA sequence for clone AI734101.

SEQ ID NO: 10813 represents the cDNA sequence for clone AI734102.

SEQ ID NO: 10814 represents the cDNA sequence for clone AI734103.

SEQ ID NO: 10815 represents the cDNA sequence for clone AI734104.

SEQ ID NO: 10816 represents the cDNA sequence for clone AI734109.

SEQ ID NO: 10817 represents the cDNA sequence for clone AI734123.

SEQ ID NO: 10818 represents the cDNA sequence for clone AI734124.

SEQ ID NO: 10819 represents the cDNA sequence for clone AI734125.

SEQ ID NO: 10820 represents the cDNA sequence for clone AI734126.

SEQ ID NO: 10821 represents the cDNA sequence for clone AI734127.

SEQ ID NO: 10822 represents the cDNA sequence for clone AI734128.

SEQ ID NO: 10823 represents the cDNA sequence for clone AI734129.

SEQ ID NO: 10824 represents the cDNA sequence for clone AI734130.

SEQ ID NO: 10825 represents the cDNA sequence for clone AI734131.

SEQ ID NO: 10826 represents the cDNA sequence for clone AI734132.

SEQ ID NO: 10827 represents the cDNA sequence for clone AI734133.

SEQ ID NO: 10828 represents the cDNA sequence for clone AI734134.

SEQ ID NO: 10829 represents the cDNA sequence for clone AI734136.

SEQ ID NO: 10830 represents the cDNA sequence for clone AI734139.

SEQ ID NO: 10831 represents the cDNA sequence for clone AI734142.

SEQ ID NO: 10832 represents the cDNA sequence for clone AI734157.

SEQ ID NO: 10833 represents the cDNA sequence for clone AI734161.

SEQ ID NO: 10834 represents the cDNA sequence for clone AI734162.

SEQ ID NO: 10835 represents the cDNA sequence for clone AI734164.

SEQ ID NO: 10836 represents the cDNA sequence for clone AI734166.

SEQ ID NO: 10837 represents the cDNA sequence for clone AI734167.

SEQ ID NO: 10838 represents the cDNA sequence for clone AI734168.

SEQ ID NO: 10839 represents the cDNA sequence for clone AI734169.

SEQ ID NO: 10840 represents the cDNA sequence for clone AI734191.

SEQ ID NO: 10841 represents the cDNA sequence for clone AI734192.

SEQ ID NO: 10842 represents the cDNA sequence for clone AI734195.

SEQ ID NO: 10843 represents the cDNA sequence for clone AI734196.

SEQ ID NO: 10844 represents the cDNA sequence for clone AI734198.

SEQ ID NO: 10845 represents the cDNA sequence for clone AI734199.

SEQ ID NO: 10846 represents the cDNA sequence for clone AI734200.

SEQ ID NO: 10847 represents the cDNA sequence for clone AI734202.

SEQ ID NO: 10848 represents the cDNA sequence for clone AI734203.

SEQ ID NO: 10849 represents the cDNA sequence for clone AI734204.

SEQ ID NO: 10850 represents the cDNA sequence for clone AI734205.

SEQ ID NO: 10851 represents the cDNA sequence for clone AI738829.

SEQ ID NO: 10852 represents the cDNA sequence for clone AI738831.

SEQ ID NO: 10853 represents the cDNA sequence for clone AI738834.

SEQ ID NO: 10854 represents the cDNA sequence for clone AI738835.

SEQ ID NO: 10855 represents the cDNA sequence for clone AI738836.

SEQ ID NO: 10856 represents the cDNA sequence for clone AI738841.

SEQ ID NO: 10857 represents the cDNA sequence for clone AI738843.

SEQ ID NO: 10858 represents the cDNA sequence for clone AI738851.

SEQ ID NO: 10859 represents the cDNA sequence for clone AI738866.

SEQ ID NO: 10860 represents the cDNA sequence for clone AI738868.

SEQ ID NO: 10861 represents the cDNA sequence for clone AI738871.

SEQ ID NO: 10862 represents the cDNA sequence for clone AI738877.

SEQ ID NO: 10863 represents an extended sequence for clone O1028C.

SEQ ID NO: 10864 represents the full length DNA sequence of clone O1029C.

SEQ ID NO: 10865 represents the cDNA sequence for clone O1003C-R.

SEQ ID NO: 10866 represents an extended sequence for clone O1030C-R.

SEQ ID NO: 10867 represents an additional extended sequence for clone O1030C-R.

SEQ ID NO: 10868 represents an extended sequence for clone O1030C.

SEQ ID NO: 10869 represents an additional extended sequence for clone O1030C.

SEQ ID NO: 10870 represents the full length DNA sequence for clone O1031C.

SEQ ID NO: 10871 represents an extended sequence for clone O1031C.

SEQ ID NO: 10872 represents the full length DNA sequence for clone O1032C.

SEQ ID NO: 10873 represents an extended sequence for clone O1032C.

SEQ ID NO: 10874 represents the full length DNA sequence of clone O1033C.

SEQ ID NO: 10875 represents an extended sequence for clone O1033C.

SEQ ID NO: 10876 represents the full length DNA sequence for clone O1034C.

SEQ ID NO: 10877 represents the DNA sequence of the 5′ end of clone O1034C.

SEQ ID NO: 10878 represents the DNA sequence of the 3′ end of the clone O1034C.

SEQ ID NO: 10879 represents an extended sequence for clone O1035C.

SEQ ID NO: 10880 represents the DNA sequence from clone O1036C.

SEQ ID NO: 10881 represents an extended sequence for clone O1036C.

SEQ ID NO: 10882 represents an additional extended sequence for clone O1036C.

SEQ ID NO: 10883 represents an additional extended sequence for clone O1036C.

SEQ ID NO: 10884 represents an additional extended sequence for clone O1036C.

SEQ ID NO: 10885 represents an extended sequence for clone O1062C.

SEQ ID NO: 10886 represents the DNA sequence of clone O1063C isolated from Ovary Chip.

SEQ ID NO: 10887 represents the DNA sequence of clone O1064C isolated from Ovary Chip.

SEQ ID NO: 10888 represents the DNA sequence of clone O1065C isolated from Ovary Chip.

SEQ ID NO: 10889 represents the DNA sequence of clone O1066C isolated from Ovary Chip.

SEQ ID NO: 10890 represents the DNA sequence of clone O1067C isolated from Ovary Chip.

SEQ ID NO: 10891 represents an extended sequence for clone O1067C.

SEQ ID NO: 10892 represents the DNA sequence of clone O1068C isolated from Ovary Chip.

SEQ ID NO: 10893 represents an extended sequence for clone O1068C.

SEQ ID NO: 10894 represents the DNA sequence for clone O1069C.

SEQ ID NO: 10895 represents the DNA sequence for clone O1070C.

SEQ ID NO: 10896 represents the DNA sequence for clone O1071C.

SEQ ID NO: 10897 represents an extended sequence for clone O1071C.

SEQ ID NO: 10898 represents the DNA sequence for clone O1072C.

SEQ ID NO: 10899 represents an extended sequence for clone.

SEQ ID NO: 10900 represents the DNA sequence for clone O1073C.

SEQ ID NO: 10901 represents an extended sequence for the clone O1073C.

SEQ ID NO: 10902 represents the DNA sequence for clone O1074C.

SEQ ID NO: 10903 represents an extended sequence for clone O1074C.

SEQ ID NO: 10904 represents the DNA sequence for clone O1075C.

SEQ ID NO: 10905 represents an extended sequence for clone O1075 C.

SEQ ID NO: 10906 represents the DNA sequence for clone O1076C.

SEQ ID NO: 10907 represents an extended sequence for clone O1076C.

SEQ ID NO: 10908 represents the DNA sequence for clone O1077C.

SEQ ID NO: 10909 represents the DNA sequence for clone O1078C.

SEQ ID NO: 10910 represents an extended sequence for clone O1078C.

SEQ ID NO: 10911 represents the DNA sequence for clone O1079C.

SEQ ID NO: 10912 represents an extended sequence for clone O1079C.

### DETAILED DESCRIPTION OF THE INVENTION

The present invention is directed generally to compositions and their use in the therapy and diagnosis of cancer, particularly ovarian cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” “is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NO: 1-10,912, or a sequence that hybridizes under moderately stringent conditions, or, alternatively, under highly stringent conditions, to a polynucleotide sequence set forth in any one of SEQ ID NO: 1-10,912.

The polypeptides of the present invention are sometimes herein referred to as ovarian tumor proteins or ovarian tumor polypeptides, as an indication that their identification has been based at least in part upon their increased levels of expression in ovarian tumor samples. Thus, an “ovarian tumor polypeptide” or “ovarian tumor protein,” refers generally to a polypeptide sequence of the present invention, or a polynucleotide sequence encoding such a polypeptide, that is expressed in a substantial proportion of ovarian tumor samples, for example preferably greater than about 20%, more preferably greater than about 30%, and most preferably greater than about 50% or more of ovarian tumor samples tested, at a level that is at least two fold, and preferably at least five fold, greater than the level of expression in normal tissues, as determined using a representative assay provided herein. An ovarian tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with ovarian cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those encoded by a polynucleotide sequence set forth in a sequence of

SEQ ID NO: 1-10,912.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GGU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Accordingly, the present invention provides methods for purifying the xenogeneic form of the tumor proteins set forth herein, such as those encoded by polynucleotide sequences set forth in

SEQ ID NO: 1-10,912.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO: 1-10,912, complements of a polynucleotide sequence set forth in any one of SEQ ID NO: 1-10,912, and degenerate variants of a polynucleotide sequence set forth in any one of SEQ ID NO: 1-10,912. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NO: 1-10,912, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompass homologous genes of xenogenic origin.

In additional embodiments, the present invention provides polynucleotide fragments comprising or consisting of various lengths of contiguous stretches of sequence identical to or complementary to one or more of the sequences disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988)Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. 1988 June 10;240(4858):1544-6; Vasanthakumar and Ahmed, Cancer Commun. 1989;1(4):225-32; Peris et al., Brain Res Mol Brain Res. Jun. 15, 1998;57(2):310-20; U.S. Pat. Nos. 5,801,154; 5,789,573; 5,718,709 and 5,610,288). Antisense constructs have also been described that inhibit and can be used to treat a variety of abnormal cellular proliferations, e.g. cancer (U.S. Pat. Nos. 5,747,470; 5,591,317 and 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. Jul. 15, 1997;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci USA. December 1987;84(24):8788-92; Forster and Symons, Cell. Apr. 24, 1987;49(2):211-20). For example, a large number of ribozymes accelerate phosphoester transfer reactions with a high degree of specificity, often cleaving only one of several phosphoesters in an oligonucleotide substrate (Cech et al., Cell. December 1981;27(3 Pt 2):487-96; Michel and Westhof, J Mol Biol. Dec. 5, 1990;216(3):585-610; Reinhold-Hurek and Shub, Nature. May 14, 1992;357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci USA. Aug. 15, 1992;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. Nucleic Acids Res. Sep. 11, 1992;20(17):4559-65. Examples of hairpin motifs are described by Hampel et al. (Eur. Pat. Appl. Publ. No. EP 0360257), Hampel and Tritz, Biochemistry Jun. 13, 1989;28(12):4929-33; Hampel et al., Nucleic Acids Res. Jan. 25, 1990;18(2):299-304 and U.S. Pat. No. 5,631,359. An example of the hepatitis δ virus motif is described by Perrotta and Been, Biochemistry. Dec. 1, 1992;31(47):11843-52; an example of the RNaseP motif is described by Guerrier-Takada et al., Cell. Decemeber 1983;35(3 Pt 2):849-57; Neurospora VS RNA ribozyme motif is described by Collins (Saville and Collins, Cell. May 18, 1990;61(4):685-96; Saville and Collins, Proc Natl Acad Sci USA. Oct. 1, 1991;88(19):8826-30; Collins and Olive, Biochemistry. Mar. 23, 1993;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells. Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol June 1997;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science Dec. 6, 1991;254(5037):1497-500; Hanvey et al., Science. Nov. 27, 1992;258(5087):1481-5; Hyrup and Nielsen, Bioorg Med Chem. January 1996;4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. April 1995;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. April 1995;3(4):437-45; Petersen et al., J Pept Sci. May-June 1995;1(3):175-83; Orum et al., Biotechniques. September 1995;19(3):472-80; Footer et al., Biochemistry. Aug. 20, 1996;35(33):10673-9; Griffith et al., Nucleic Acids Res. Aug. 11, 1995;23(15):3003-8; Pardridge et al., Proc Natl Acad Sci USA. June 6, 1995;92(12):5592-6; Boffa et al., Proc Natl Acad Sci USA. Mar. 14, 1995;92(6):1901-5; Gambacorti-Passerini et al., Blood. Aug. 15, 1996;88(4):1411-7; Armitage et al., Proc Natl Acad Sci USA. Nov. 11, 1997;94(23):12320-5; Seeger et al., Biotechniques. September 1997;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993;65(24):3545-9) and Jensen et al. (Biochemistry. Apr. 22, 1997;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, W H Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as ovarian cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. Nos. 5,240,856; 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

T Cell Receptor Compositions The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJ_{β} exon is transcribed and spliced to join to a C_{β}. For the α chain, a V_{α} gene segment rearranges to a J_{α} gene segment to create the functional exon that is then transcribed and spliced to the C_{α}. Diversity is further increased during the recombination process by the random addition of P and N-nucleotides between the V, D, and J segments of the β chain and between the V and J segments in the α chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a_tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of ovarian cancer as discussed further below.

In further aspects of the present invention, cloned TCRs specific for a polypeptide recited herein may be used in a kit for the diagnosis of ovarian cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa l saponins . Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula (I)

HO(CH₂CH₂O)ₙ—A—R,

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12ₜₕ edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD 11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature Mar. 27, 1997;386(6623):410-4; Hwang et al., Crit Rev Ther Drug Carrier Syst 1998;15(3):243-84; U.S. Pat. Nos. 5,641,515; 5,580,579 and 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. Nos. 5,543,158; 5,641,515 and 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g, glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. Nos. 5,756,353 and 5,804,212. Likewise, the delivery of drugs using intranasal microparticle resins (Takenaga et al., J Controlled Release Mar. 2, 1998;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol July 1998;16(7):307-21; Takakura, Nippon Rinsho 1998 March;56(3):691-5; Chandran et al., Indian J Exp Biol. August 1997;35(8):801-9; Margalit, Crit Rev Ther Drug Carrier Syst. 1995;12(2-3):233-61; U.S. Pat. Nos. 5,567,434; 5,552,157; 5,565,213; 5,738,868 and 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. Sep. 25, 1990;265(27):16337-42; Muller et al., DNA Cell Biol. April 1990;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. December 1998;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; zur Muhlen et al., Eur J Pharm Biopharm. March 1998;45(2):149-55; Zambaux et al. J Controlled Release. Jan. 2, 1998;50(1-3):31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001;60(1):1-7; Renner, et al., Ann Hematol December 2000;79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly ovarian cancer cells, offer a powerful approach for inducing immune responses against ovarian cancer, and are an important aspect of the present invention.

Therefore, in further aspects of the present invention, the pharmaceutical compositions described herein may be used to stimulate an immune response against cancer, particularly for the immunotherapy of ovarian cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more ovarian tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as ovarian cancer. In addition, such proteins may be useful for the detection of other cancers. The binding agents provided herein generally permit detection of the level of antigen that binds to the agent in the biological sample.

Polynucleotide primers and probes may be used to detect the level of mRNA encoding a tumor protein, which is also indicative of the presence or absence of a cancer. In general, a tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g. PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as ovarian cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100% -specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺T cells, activation is preferably detected by evaluating proliferation of the T cells. For CD8⁺T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis.

Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing ovarian tumor antigens. Detection of ovarian cancer cells in biological samples, e.g., bone marrow samples, peripheral blood, and small needle aspiration samples is desirable for diagnosis and prognosis in ovarian cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for ovarian tumor antigens can be generated and used in a similar manner. For example, mAbs that bind to tumor-specific cell surface antigens may be conjugated to magnetic beads, or formulated in a tetrameric antibody complex, and used to enrich or positively select metastatic ovarian tumor cells from a sample. Once a sample is enriched or positively selected, cells may be lysed and RNA isolated. RNA may then be subjected to RT-PCR analysis using ovarian tumor-specific primers in a real-time PCR assay as described herein. One skilled in the art will recognize that enriched or selected populations of cells may be analyzed by other methods (e.g. in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Identification of Ovarian Tumor Protein cDNAS

This Example illustrates the identification of cDNA molecules encoding ovarian tumor proteins.

Ovarian-specific genes were identified by electronic subtraction. The method used was similar to that described by Vasmatizis et al., Proc. Natl. Acad. Sci. USA 95:300-304, 1998, but there were several key differences. The sequences of EST clones (1,453,679) were downloaded from the GenBank public human EST database. Human cDNA libraries were downloaded to create a database of these cDNA libraries and the EST sequences derived from them. The cDNA libraries were grouped into three groups: Plus, Minus and Other/Neutral. The Plus group included 26 libraries constructed from ovary normal and tumor tissues (and therefore including those containing ovary-specific ESTs); the Minus group consisted of 184 libraries derived from all adult normal tissues except for breast, uterus and male-specific tissues, such as prostate; the Other/Neutral group contained libraries from tissues where expression is considered irrelevant (e.g., fetal tissue, tumors, cell lines other than ovary cell lines, non-relevant tissues. A total of 43,581 ESTs were derived from the 26 available ovary libraries (this subset represents 3% of the total 1,453,679 ESTs in the database). These ESTs were preprocessed to remove common sequence repeats and cloning adapters, resulting in a final Plus group of 39,019 (a decrease of 10%).

Each Plus group (ovary) EST sequence was used as a query “seed” sequence in a BLASTN (version 2.0.9; May 7, 1999) search against the total human EST database. Standard measures of similarity are insufficient in this sort of analysis, as EST relationships often include short stretches and poor sequence data. Criteria employed in this study required a matching segment to be at least 75 nucleotides in length, and the density of exact matches within this segment to be at least 80%. This was considered conservative criteria designed to avoid short spurious matches while allowing for polymorphisms and errors in sequencing. Each BLAST search generated a cluster of related sequences based on direct overlap with the query “seed” sequence. A second level of clustering was performed to merge closely related clusters and to eliminate redundancy resulting from the fact that similar clusters are generated if the clusters contain more than one seed (i.e., sequences from the Plus EST group). The resulting “super clusters” were discarded if they grew in size to 200 or more ESTs, since these probably represented repetitive elements that were not removed by the initial preprocessing of the seeds, or highly expressed genes such as those for ribosomal proteins. Superclusters were merged if they shared at least one third of their sequences.

The BLAST searches gave rise to a total of 38,979 clusters. In the first super clustering stage, 10,307 clusters grew beyond the limit of 200 clones. Following this step and the elimination of duplicates, there were 15,758 super clusters remaining. This number was reduced to 15,244 after adjacent clusters were merged. Resulting super clusters were analyzed to determine the tissue source of each EST clone contained within it and this expression profile was used to classify the superclusters into four groups: Type 1—this supercluster contains EST clones found in the Plus group only, with no expression in the Minus or Other/Neutral group libraries; Type 2—EST clones in the supercluster are found in the Plus and Other/Neutral group libraries, with no expression in the Minus group; Type 3—super cluster EST clones found in all groups, but the number of ESTs in the Plus group is higher than in either of the Minus or Other/Neutral groups; Type 4—super cluster EST clones found in all groups, but the number in the Plus group is higher than in the Minus group with expression in the Other/Neutral group non relevant. Sequences derived from the Plus library group that were placed in Types 1, 2 and 3 superclusters are provided herein as SEQ ID NO:1-10,862. The electronic subtraction procedures identified these sequences as having significant differential expression in ovary tissue.

Sequences which showed good electronic subtraction profiles are described in more detail in Table 2.

TABLE 2
Electronic Subtraction Analysis

| Clone ID | | | | | | |
|---|---|---|---|---|---|---|
| IMAGE # | Accession # | SuperClstr | Plus | Minus | Neutral | EST/GenBank Annotation |
| 595243 | AA164216/ | AA164216 | 1 | 0 | 8 | Novel |
| | AA164215 | | | | | |
| 594484 | AA180009/ | AA164850 | 2 | 0 | 27 | Reverse transcriptase/human |
| | AA164745 | | | | | endogenous retrovirus |
| 843028 | AA488541/ | AI567987 | 15 | 0 | 22 | Megakaryocyte potentiating |
| | AA488406 | | | | | factor; mesothelin or CAK1 |
| 76058 | T59567/ | T59567 | 1 | 0 | 12 | ALEX3, armadillo repeat |
| | T59521 | | | | | containing protein |
| 544863 | AA075131/ | AA075131 | 1 | 0 | 0 | Cathepsin L, cysteine protease |
| | AA075149 | | | | | (major excreted protein, MEP) |
| 741139 | AA402754/ | AA402207 | 1 | 0 | 3 | Developmental protein eyes |
| | AA402207 | | | | | absent (Eab1) |
| 545242 | AA076182/ | AA180012 | 4 | 0 | 16 | STAT1, signal transducer/ |
| | AA076085 | | | | | activator of transcription 1 |
| 75673 | T58484 | T58484 | 1 | 0 | 0 | Genomic clone, chromosome 21 |
| 811600 | AA458533/ | AA454609 | 1 | 0 | 13 | Forkhead transcription factor |
| | AA454609 | | | | | HFH-4 |
| 739457 | AA477250/ | AA402253 | 2 | 0 | 3 | Novel |
| | AA477249 | | | | | |
| 77262 | T50256/ | T50256 | 1 | 0 | 0 | Vector, bacteriophage lambda |
| | T50206 | | | | | lacZ |
| 77257 | T50212 | T50212 | 1 | 0 | 0 | Aspartate aminotransferase; |
| | | | | | | genomic E. coli clones U/K |
| | | | | | | protein |
| 74768 | T47079 | T47079 | 1 | 0 | 0 | Genomic clone, chromosome |
| | | | | | | 13q12.11-12.3 |
| 593444 | AI733904/ | AA160258 | 1 | 0 | 0 | Novel |
| | AI732611/ | | | | | |
| | AA160259/ | | | | | |
| | AA160258 | | | | | |
| 811063 | AI734202/ | AA485451 | 1 | 0 | 1 | DNA-binding protein A |
| | AI732823/ | | | | | (HUMBPA) |
| | AA485619/ | | | | | |
| | AA485451 | | | | | |
| 593218 | AI733902/ | AA159743 | 1 | 0 | 1 | Novel |
| | AI732609/ | | | | | |
| | AA159613/ | | | | | |
| | AA159743 | | | | | |
| 595232 | AI732624/ | AA173518 | 1 | 0 | 0 | Genomic clone, chromosome |
| | AA173518 | | | | | 12p11-37.2-54.4 |
| 75759 | T58522 | T58522 | 2 | 0 | 16 | cDNA clone FLJ10748, weakly |
| | | | | | | similar to ringcanal protein |
| 714232 | AA293601 | AA293601 | 1 | 0 | 2 | Novel |
| 809858 | AA464305/ | AI567829 | 2 | 0 | 17 | COP9 complex subunit 7a |
| | AA455119 | | | | | |
| 489076 | AA057195/ | AA423796 | 1 | 0 | 5 | Elastin microfibril interfase |
| | AA057129 | | | | | located protein (EMI); |
| | | | | | | ketohexokinase |
| 771328 | AA476222/ | AA476222 | 2 | 0 | 6 | MG81 |
| | AA476223 | | | | | |
| 755093 | AI821682/ | AA482508 | 1 | 0 | 0 | Neuronal apoptosis inhibitory |
| | AI820932/ | | | | | protein; basic transn factor 2 p44 |
| | AA482660/ | | | | | |
| | AA482508 | | | | | |
| 771053 | AA430718/ | AA430718 | 1 | 0 | 2 | Zinc Finger Protein 26; contains |
| | AA427528 | | | | | element THR, repetitive element |
| 77250 | T50141 | T50141 | 7 | 2 | 1 | Excisionase (xis) and integrase |
| | | | | | | (int) bacteriophage genes |
| 810109 | AA465048/ | AA292245 | 2 | 0 | 9 | cDNA clone DKFZp434K022 |
| | AA464979 | | | | | |
| 136984 | AF074992/ | AA401975 | 3 | 0 | 2 | cDNA YH90A09 |
| | R35849 | | | | | |
| 809776 | AA454784/ | AA454732 | 3 | 0 | 19 | IL-16, lymphocyte |
| | AA454732 | | | | | chemoattractant factor |
| 593439 | AA165668/ | AA165632 | 3 | 2 | 31 | GAG polyprotein/endogenous |
| | AA165632 | | | | | retrovirus sequences |
| 76081 | T59590 | T59590 | 1 | 0 | 0 | Novel |
| 724886 | AA404613/ | AA464208 | 5 | 0 | 6 | contains PTR7 repetitive |
| | AA291468 | | | | | element; T cell receptor gamma |
| | | | | | | V1? |
| 724962 | AA404631/ | AA402723 | 15 | 0 | 38 | Pregnancy-Associated |
| | AA291495 | | | | | endometrial alpha2 globulin; = |
| | | | | | | HPP14 |
| 283151 | N45230 | AA284811 | 2 | 0 | 6 | Genomic clones, alpha albumin |
| | | | | | | and MER27 repetitive element |
| 595363 | AA164920/ | AA402031 | 3 | 1 | 30 | Interferon-gamma induced small |
| | AA164919 | | | | | cytokine subfamily B #10 |
| 544548 | AA075105/ | AI567987 | 15 | 0 | 22 | Megakaryocyte potentiating |
| | AA074952 | | | | | factor; mesothelin or CAK1 |
| 595220 | AA164851/ | AA164850 | 2 | 0 | 27 | Genomic clones |
| | AA164850 | | | | | |
| 545443 | AA079191/ | AA167449 | 5 | 1 | 55 | Genomic clone containing X |
| | AA079190 | | | | | (inactive)-specific transcript |
| | | | | | | (XIST) |
| 545185 | AA076101 | AA076101 | 1 | 0 | 0 | Acidic ribosomal phosphoprotein |
| | | | | | | P0 |
| 714080 | AA284545/ | AA284815 | 1 | 0 | 0 | Megakaryocyte potentiating |
| | AA284815 | | | | | factor; mesothelin or CAK1 |
| 595449 | AA173739/ | AA173383 | 5 | 0 | 33 | Neurotensin receptor; C protein- |
| | AA173383 | | | | | coupled receptor (GPR39) |
| | AA176693/ | AA173996 | 2 | 1 | 3 | Pak1, p21-activated serine/ |
| | AA173996 | | | | | threonine-protein kinase (JNK |
| | | | | | | path) |
| | AI821564/ | AA284658 | 1 | 0 | 1 | Novel |
| | AA284659/ | | | | | |
| | AA284658 | | | | | |
| | AA075034/ | AA169452 | 2 | 1 | 27 | C. elegans homolog, CGI-19 |
| | AA075033 | | | | | |
| | AA394090/ | AA293773 | 1 | 0 | 6 | cDNA clone 23870 |
| | AA293773 | | | | | |
| | T57605 | T57605 | 1 | 0 | 0 | Novel |
| | AA456327/ | AA480796 | 2 | 1 | 18 | Genomic clone, chromosome 22 |
| | AA454681 | | | | | |
| | AA164782/ | AA164644 | 3 | 0 | 15 | Novel |
| | AA164781 | | | | | |
| | AA166898/ | AA166756 | 1 | 0 | 0 | Mixed genomic clones, contains |
| | AA166756 | | | | | Alu repetitive element |
| | AA075203/ | AA075202 | 1 | 0 | 1 | E. coli genomic DNA; ycbS, a |
| | AA075202 | | | | | putative outer membrane protein |
| | AA405395/ | AA402723 | 15 | 0 | 38 | Pregnancy-Associated |
| | AA402883 | | | | | endometrial alpha2 globulin; = |
| | | | | | | HPP14 |
| | AA172236/ | AA172056 | 1 | 0 | 4 | Genomic clone, chromosome 22 |
| | AA172056 | | | | | (cat eye syndrome region) |
| | T57611/ | T57557 | 1 | 0 | 0 | Novel |
| | T57557 | | | | | |
| | AA419214/ | AA173383 | 5 | 0 | 33 | Neurotensin receptor; G protein |
| | AA419229 | | | | | coupled receptor (GPR39) |
| | AA171974/ | AA171755 | 1 | 0 | 3 | Genomic clone, chromosome 14 |
| | AA171755 | | | | | |
| | AA076270/ | AA076269 | 1 | 0 | 0 | Novel |
| | AA076269 | | | | | |
| | AA464689 | AA464689 | 1 | 0 | 9 | Novel |
| | AA456446/ | AA477508 | 3 | 0 | 38 | Myo-inositol 1-phosphate |
| | AA454554 | | | | | synthase |
| | T60090 | T60090 | 1 | 0 | 0 | Genomic clone, chromosome 1 |
| | AA075211/ | AA074946 | 2 | 0 | 0 | Nucleoside diphosphate kinase |
| | AA075126 | | | | | (DR-nm23); Ndk3-like |
| | AA076228/ | AA076228 | 1 | 0 | 3 | C. elegans homolog, CGI-62 |
| | AA076227 | | | | | |
| | AA075310 | AA075310 | 1 | 0 | 9 | Sperm membrane protein; |
| | | | | | | nucleoporin; Ran-binding protein |
| | | | | | | 2 |
| | T58551/ | T58501 | 1 | 0 | 36 | Genomic clone, chromosome |
| | T58501 | | | | | 22q13.31-13.33 |
| | T59551 | T59551 | 1 | 0 | 0 | Reverse transcriptase-like protein |
| | | | | | | -rat |
| | AA464691 | AA405746 | 2 | 0 | 25 | Genomic clone, chromosome |
| | | | | | | Xp22 |
| | T52869/ | T52868 | 1 | 0 | 0 | Contains repetitive DNA |
| | T52868 | | | | | |
| | AF147380/ | T59850 | 2 | 0 | 0 | cDNA clone YB67A06, contains |
| | T59906/ | | | | | CER/MER22 repetitive element |
| | T59850 | | | | | |
| 2216507 | AI653489 | AI653489 | 1 | 0 | 1 | gDNA 12p13.3 PAC RPCI5- |
| | | | | | | 940J5 |
| 2292394 | AI648506 | AI648506 | 2 | 0 | 54 | PTK7 protein tyrosine kinase 7 |
| | | | | | | (PTK7), mRNA |
| 2216151 | AI654462 | AI654452 | 2 | 0 | 3 | |
| 981215 | AA526423 | AA526423 | 1 | 0 | 1 | |
| 1373880 | AA828788 | AA828788 | 1 | 0 | 0 | gDNA chromosome 5 clone |
| | | | | | | CTD-2318A17 |
| 1373112 | AA837621 | AA837621 | 1 | 0 | 4 | |
| 2002493 | AI224233 | AI223766 | 10 | 0 | 37 | |
| 1373281 | AA827974 | AA526119 | 2 | 0 | 0 | gDNA clone 1018D12 20q11.1- |
| | | | | | | 11.22 |
| 2216154 | AI654454 | AI567994 | 2 | 0 | 8 | VSGP/F-spondin, complete cds |
| 755378 | AA410595 | AA410247 | 3 | 0 | 1 | Inhibitor of DNA binding 4, |
| | | | | | | dom/neg HLH protein (ID4), |
| 769666 | AA428329 | AA291377 | 2 | 0 | 2 | gDNA RP11-343B21 from 2 |
| 2216636 | AI653555 | AI653555 | 1 | 0 | 1 | gDNA RP5-1185I7 from |
| | | | | | | 7q11.23-q21 |
| 1455124 | AA909822 | AA909822 | 1 | 0 | 19 | mRNA for GARS-AIRS-GART |
| 1374123 | AA828611 | AA828611 | 1 | 0 | 0 | |
| 770437 | AA427513 | AA427513 | 2 | 0 | 4 | gDNA chromosome 8 clone |
| | | | | | | RP11-4K16 |
| 739389 | AA476860 | AA856964 | 3 | 1 | 26 | TLS-associated Ser-Arg protein 1 |
| | | | | | | (TASR1), mRNA |
| 1985442 | AI254625 | AI254625 | 1 | 0 | 4 | |
| 981039 | AA526017 | AA526017 | 1 | 0 | 40 | Smad1 mRNA |
| 755111 | AA411372 | AA411372 | 1 | 0 | 15 | mRNA for KIAA0326 gene |
| 1965439 | AI356599 | AA862214 | 2 | 0 | 28 | cDNA FLJ10943 fis, clone |
| | | | | | | OVARC1001360 |
| 755436 | AA419044 | AA292435 | 3 | 0 | 23 | cDNA FLJ10023 fis, clone |
| | | | | | | HEMBA1000608 |
| 742144 | AA405975 | AA405727 | 2 | 0 | 0 | |
| 1373104 | AA837606 | AA828060 | 3 | 0 | 0 | gDNA clone B331M8 map 4q25 |
| 1373878 | AA828778 | AA828778 | 1 | 0 | 0 | CREB binding protein |
| | | | | | | (Rubinstein-Taybi |
| | | | | | | syndrome)(CREBBP |
| 1454766 | AA908470 | AA908470 | 1 | 0 | 10 | gDNA clone RP11-83M17 from |
| | | | | | | 7q31 |
| 1373719 | AA828882 | AA828882 | 2 | 0 | 0 | |
| 1374009 | AA828703 | AA828138 | 2 | 0 | 4 | hepatocellular carcinoma antigen |
| | | | | | | 519 (HCA519) |
| 1373380 | AA828138 | AA828138 | 2 | 0 | 4 | hepatocellular carcinoma antigen |
| | | | | | | 519 (HCA519) |
| 1373381 | AA828148 | AA828038 | 2 | 0 | 0 | gDNA clone RP3-525L6 on |
| | | | | | | 6p22.3- |
| 1964546 | AI284862 | AI284862 | 1 | 0 | 1 | gDNA clone RP11-215O15 on |
| | | | | | | chromosome 9 |
| 1192366 | AA650614 | AA650614 | 2 | 0 | 0 | |
| 2260607 | AI590617 | AI590617 | 1 | 0 | 5 | Staufen (Drosophila, RNA- |
| | | | | | | binding protein) (STAU) |
| 1154834 | AA642166 | AA642166 | 1 | 0 | 0 | |
| 981113 | AA526205 | AA526205 | 1 | 0 | 0 | |
| 2292558 | AI648453 | AI648453 | 1 | 0 | 0 | |
| 981376 | AA525937 | AA525937 | 1 | 0 | 0 | |
| 1374125 | AA828612 | AA828612 | 1 | 0 | 0 | |
| 1374171 | AA828647 | AA828647 | 1 | 0 | 0 | eIF3 p40 subunit gene, exon 7 |
| 1965552 | AI364864 | AI364864 | 1 | 0 | 8 | gDNA clone RP5-850O15 on |
| | | | | | | 1p32.3-34.2 |
| 981310 | AA525800 | AA525800 | 1 | 0 | 0 | |
| 1373500 | AA847986 | AA829098 | 2 | 0 | 0 | |
| 1373375 | AA828135 | AA828135 | 2 | 1 | 10 | zinc finger protein (ZFD25) |
| | | | | | | mRNA |
| 1190617 | AA650078 | AA650078 | 1 | 0 | 0 | |
| 2292555 | AI648459 | AI648459 | 1 | 0 | 9 | mitosin mRNA |
| 755357 | AA410276 | AA293518 | 2 | 0 | 4 | mRNA for KIAA1335 protein |
| 1373287 | AA827977 | AA827977 | 1 | 0 | 4 | |
| 1192390 | AA650618 | AA650614 | 2 | 0 | 0 | |
| 2260515 | AI590596 | AI590596 | 3 | 0 | 4 | gDNA chromosome 5 clone |
| | | | | | | CTD-2019M3 |
| 981372 | AA525935 | AA525935 | 2 | 1 | 3 | peroxisomal 70 kD memb |
| | | | | | | protein mRNA |
| 980932 | AA525774 | AA525774 | 1 | 0 | 0 | gDNA clone B271E1 on 4q25 |
| 1374020 | AA828698 | AA828797 | 5 | 0 | 6 | gDNA chromosome 20 clones 97 |
| | | | | | | and 127 |
| 1373311 | AA828039 | AA828039 | 1 | 0 | 3 | gDNA chromosome 21 segment |
| | | | | | | HS21C010 |
| 1047907 | AA773935 | AA362728 | 2 | 0 | 11 | cDNA: FLJ21661 fis, clone |
| | | | | | | COL08837 |
| 1154937 | AA627343 | AA627343 | 1 | 0 | 0 | |
| 1373504 | AA847988 | AA847988 | 1 | 0 | 3 | gDNA 12 BAC RP11-588G21 |
| 1963836 | AI280530 | AI280530 | 1 | 0 | 0 | |
| 981337 | AA525918 | AA525918 | 1 | 0 | 0 | gDNA chromosome 21 segment |
| | | | | | | HS21C046 |
| 1373879 | AA828787 | AA828787 | 2 | 0 | 1 | |
| 980966 | AA525874 | AA525874 | 1 | 0 | 0 | gDNA BAC clone CTB-95A14 |
| | | | | | | from 7p15-p21 |
| 2292391 | AI648510 | AI648510 | 1 | 0 | 0 | SYBL1 gene, exons 6-8 |
| 2292584 | AI648468 | AI648468 | 1 | 0 | 6 | mRNA for KIAA1442 protein, |
| | | | | | | partial cds |
| 981068 | AA526095 | AA525221 | 2 | 0 | 0 | |
| 1373285 | AA827976 | AA827976 | 2 | 0 | 2 | |
| 1373419 | AA826312 | AA826312 | 1 | 0 | 0 | |
| 1374172 | AA828640 | AA828640 | 2 | 0 | 0 | |
| 1373400 | AA826292 | AA826292 | 1 | 0 | 0 | AMP-activated protein kinase |
| | | | | | | (hAMPK) |
| 1373695 | AA828597 | AA828597 | 1 | 0 | 1 | gDNA BAC 55C20 on |
| | | | | | | chromosome 6. |
| 1373807 | AA828690 | AA828690 | 1 | 0 | 0 | |
| 1373415 | AA826310 | AA826310 | 1 | 0 | 0 | |
| 981359 | AA525929 | AA525929 | 2 | 0 | 13 | gDNA, chromosome 21q, section |
| | | | | | | 89/105 |
| 1373309 | AA828038 | AA828038 | 2 | 0 | 0 | gDNA clone RP3-525L6 on |
| | | | | | | 6p22.3- |
| 2260575 | AI591141 | AI591141 | 1 | 0 | 3 | protein Tyr phosphatase, receptor |
| | | | | | | type, M (PTPRM) |
| 755715 | AA496499 | AA496499 | 2 | 0 | 2 | platelet derived growth factor C |
| | | | | | | (PDGFC) |
| 809959 | AA454835 | AA430746 | 3 | 0 | 29 | EWS protein/E1A enhancer |
| | | | | | | binding protein |
| 1373509 | AA848000 | AA829138 | 3 | 0 | 0 | |
| 1374033 | AA829114 | AA828882 | 2 | 0 | 0 | |
| 1373080 | AA837588 | AA837588 | 1 | 0 | 4 | gDNA 3 BAC RP11-91B3 |
| 1373691 | AA828596 | AA828596 | 1 | 0 | 15 | gDNA clone 747H23 on 6q13-15 |
| 1964868 | AI355523 | AI355523 | 1 | 0 | 3 | gDNA BAC clone GS1-67A24 |
| | | | | | | from 7q21.q21.2 |
| 1190662 | AA650084 | AA650084 | 1 | 0 | 9 | phospholipase C-beta-3 (PLCB3) |
| | | | | | | gene |
| 1154908 | AA627330 | AA627330 | 1 | 0 | 0 | |
| 1985131 | AI242020 | AI242020 | 1 | 0 | 3 | Ca2+-activated potassium |
| | | | | | | channel SK3 |
| 1985090 | AI241990 | AI241990 | 7 | 0 | 1 | |
| 1985420 | AI254602 | AI252922 | 4 | 0 | 1 | |
| 1985633 | AI254657 | AI254657 | 1 | 0 | 0 | gDNA clone RP3-421H16 on |
| | | | | | | Xp11.3-21. |
| 1985494 | AI254938 | AI254938 | 2 | 0 | 1 | gDNA chromosome 14 BAC R- |
| | | | | | | 131H24 |
| 1985584 | AI251343 | AI254785 | 2 | 0 | 1 | gDNA GS1-304P7 on 1q25.1- |
| | | | | | | 31.1 |
| 1985071 | AI241981 | AI241981 | 1 | 0 | 0 | mRNA KIAA0741 gene product |
| | | | | | | (IF2) |
| 1985548 | AI251317 | AA425733 | 4 | 0 | 25 | gDNA on 7q22 |
| 1985096 | AI241992 | AI241992 | 1 | 0 | 0 | gDNA clone RP13-206I21 on |
| | | | | | | Xp11.3-21.1 |
| 1985539 | AI251255 | AI251255 | 1 | 0 | 0 | |
| 1985503 | AI254963 | AI254963 | 1 | 0 | 0 | |
| 1985111 | AI242006 | AI242006 | 2 | 0 | 2 | CLNS1A gene, intron 1 |
| 1985085 | AI241987 | AI241987 | 1 | 0 | 0 | mRNAKIAA0741 gene product |
| | | | | | | (IF2) |
| 810330 | AA464144 | AA428222 | 2 | 0 | 3 | cytochrome B561, HCYTO |
| | | | | | | B561 |
| 980920 | AA525758 | AA525758 | 1 | 0 | 0 | cDNA FLJ10145 fis, clone |
| | | | | | | HEMBA1003322 |
| 1985162 | AI254100 | AI254100 | 2 | 0 | 4 | Lens epithelium-derived growth |
| | | | | | | factor |
| 930200 | AA502530 | AA502530 | 1 | 0 | 0 | |
| 1985535 | AI251253 | AI251253 | 1 | 0 | 2 | cDNA DKFZp586B0319 |
| 1985593 | AI251369 | AI251369 | 1 | 0 | 0 | |
| 2003171 | AI250559 | AI250559 | 1 | 0 | 6 | NY-REN-41 antigen mRNA |
| 810462 | AA457130 | AA457130 | 3 | 0 | 0 | gDNA 8q21.3:RICK gene |
| 1985079 | AI241985 | AI241985 | 1 | 0 | 0 | mRNA KIAA0741 gene product |
| | | | | | | (IF2) |
| 769684 | AA428757 | AA425850 | 4 | 1 | 17 | |
| 1985127 | AI242019 | AI144024 | 7 | 0 | 3 | |
| 1985630 | AI254644 | AI254644 | 1 | 0 | 0 | gDNA, chromosome 21q, section |
| | | | | | | 69/105 |
| 1985576 | AI251339 | AI251339 | 1 | 0 | 1 | |
| 1985558 | AI251322 | AI251322 | 1 | 0 | 0 | AF-4 gene, exons 2 to 7 and Alu |
| | | | | | | repeats |
| 1984612 | AI255127 | AI254106 | 2 | 0 | 2 | |
| 1373305 | AA828036 | AA828036 | 1 | 0 | 0 | |
| 2002838 | AI224248 | AI054405 | 4 | 0 | 14 | |
| 1984571 | AI251722 | AI251722 | 1 | 0 | 0 | |
| 1985520 | AI251235 | AI251235 | 2 | 0 | 3 | GAC-1 (GAC-1) mRNA |
| 1985272 | AI254763 | AI254763 | 1 | 0 | 0 | |
| 2002656 | AI223675 | AI252423 | 4 | 2 | 32 | gDNA chromosome X region |
| 1985594 | AI251359 | AI054387 | 8 | 0 | 4 | |
| 1985474 | AI254929 | AI254929 | 1 | 0 | 0 | mRNA KIAA0741 gene product |
| | | | | | | (IF2) |
| 2119169 | AI401777 | AA363377 | 1 | 0 | 1 | |
| 755480A | A410639 | AA410639 | 1 | 0 | 2 | |
| 1985049 | AI251638 | AI251638 | 1 | 0 | 0 | |
| 2006525 | AI265778 | AI265778 | 1 | 0 | 35 | cDNA FLJ11653 fis, clone |
| | | | | | | HEMBA1004538 |
| 1984955 | AI252857 | AI252857 | 1 | 0 | 0 | mRNA KIAA0741 gene product |
| | | | | | | (IF2) |
| 810467 | AA457141 | AA457141 | 1 | 0 | 0 | |
| 993046 | AA570694 | AA363055 | 1 | 0 | 4 | hypothetical protein FLJ10099 |
| | | | | | | (FLJ10099) |
| 810530 | AA464548 | AA464548 | 3 | 2 | 25 | Claudin 7 (CLDN7) |
| 1985621 | AI254651 | AI254651 | 1 | 0 | 0 | |
| 2002637 | AI223666 | AI223666 | 1 | 0 | 2 | |
| 755865 | AA496479 | AA496479 | 1 | 0 | 4 | gDNA chromosome 21, NF1- |
| | | | | | | related locus |
| 1985448 | AI254906 | AI254906 | 1 | 0 | 0 | |
| 2292434 | AI648563 | AI648563 | 2 | 0 | 0 | gDNA 12p12-21.3-21.8 |
| 810498 | AA457154 | AA457154 | 1 | 0 | 3 | gDNA clone RP11-379I13 on |
| | | | | | | chromosome 10 |
| 2003068 | AI223766 | AI223766 | 10 | 0 | 37 | |
| 2006294 | AI279388 | AI279388 | 1 | 0 | 8 | Integin, alpha 9 (ITGA9), mRNA |
| 1985556 | AI251321 | AI251321 | 1 | 0 | 1 | |
| 1985494 | AI254938 | AI254938 | 2 | 0 | 1 | gDNA chromosome 14 clone |
| | | | | | | BAC R-131H24 |
| 1985040 | AI251623 | AI254571 | 3 | 0 | 1 | |
| 1984903 | AI254586 | AI053894 | 50 | 5 | 68 | |
| 2292460 | AI648530 | AI648530 | 1 | 0 | 1 | Wilms tumor associated protein |
| | | | | | | (WIT-1) |
| 1984941 | AI252841 | AI252841 | 2 | 1 | 3 | gDNA chromosome 14 clone |
| | | | | | | BAC R-804L24 |
| 770747 | AA454520 | AA454520 | 1 | 0 | 0 | |
| 1985061 | AI251642 | AI251642 | 1 | 0 | 0 | |
| 2260925 | AI591032 | AI591032 | 1 | 0 | 4 | |
| 2006359 | AI264852 | AI264852 | 1 | 0 | 33 | |
| 2292490 | AI648545 | AI648545 | 1 | 0 | 13 | |
| 770522 | AA456762 | AA456762 | 1 | 0 | 16 | |
| 2292472 | AI648533 | AI648533 | 1 | 0 | 0 | |
| 2002584 | AI223506 | AI223506 | 1 | 0 | 0 | |
| 2006508 | AI265914 | AI265914 | 1 | 0 | 4 | |
| 2002702 | AI223706 | AI223706 | 1 | 0 | 0 | |
| 2002948 | AI223609 | AI053964 | 5 | 0 | 13 | |
| 2002540 | AI223470 | AI223470 | 1 | 0 | 0 | |
| 2260893 | AI591004 | AI591004 | 1 | 0 | 2 | |
| 2003136 | AI250523 | AI250523 | 1 | 0 | 0 | |
| 2260680 | AI590757 | AI590757 | 1 | 0 | 1 | |
| 2219675 | AI738841 | AI738841 | 1 | 0 | 0 | |
| 2006285 | AI279392 | AI279392 | 1 | 0 | 70 | |
| 1984994 | AI252969 | AI252969 | 1 | 0 | 3 | |
| 2260809 | AI608710 | AI608710 | 1 | 0 | 0 | |
| 2260492 | AI590577 | AI590577 | 1 | 0 | 1 | |
| 1964644 | AI287912 | AI287912 | 1 | 0 | 0 | |
| 770961 | AA428171 | AA428171 | 1 | 0 | 1 | |
| 810067 | AA455305 | AA165584 | 8 | 7 | 55 | |
| 2107496 | AI380369 | AA362711 | 1 | 0 | 2 | |
| 2292567 | AI648462 | AI648462 | 1 | 0 | 1 | |
| 2260591 | AI590610 | AI590610 | 1 | 0 | 0 | |
| 2261022 | AI591055 | AI591055 | 1 | 0 | 2 | |
| 2292702 | AI612846 | AA167381 | 2 | 0 | 21 | |
| 2002858 | AI224270 | AA166620 | 3 | 0 | 17 | |
| 2006307 | AI279408 | AI279408 | 1 | 0 | 8 | |
| 1984931 | AI252836 | AI252836 | 1 | 0 | 35 | |
| 2260917 | AI591022 | AI591022 | 1 | 0 | 0 | |
| 770927 | AA433865 | AA433865 | 1 | 0 | 8 | |
| 2006292 | AI279387 | AI279387 | 1 | 0 | 2 | |
| 2261010 | AI609587 | AI609587 | 2 | 0 | 14 | |
| 741039 | AA402296 | AA402296 | 1 | 0 | 0 | |
| 2006364 | AI264845 | AI264845 | 1 | 0 | 14 | |
| 1985021 | AI251613 | AI251613 | 1 | 0 | 0 | |
| 1374360 | AA828959 | AA829097 | 3 | 0 | 1 | |
| 1985005 | AI252985 | AI252985 | 1 | 0 | 1 | |
| 2258189 | AI623135 | AF136413 | 1 | 0 | 1 | |
| 2006320 | AI279404 | AI279404 | 1 | 0 | 6 | |
| 770715 | AA454497 | AA454497 | 1 | 0 | 9 | |
| 2261186 | AI609337 | AI609337 | 1 | 0 | 16 | |
| 2216400 | AI654744 | AI654744 | 1 | 0 | 16 | |
| 2006496 | AI265908 | AI265908 | 1 | 0 | 0 | |
| 2292705 | AI612857 | AI612857 | 1 | 0 | 0 | |
| 1964750 | AI283331 | AI283331 | 1 | 0 | 0 | |
| 2260699 | AI590770 | AI590770 | 1 | 0 | 9 | |
| 1984950 | AI252845 | AI254780 | 5 | 0 | 2 | |
| 770978 | AA428247 | AA428247 | 2 | 1 | 12 | |
| 770998 | AA428253 | AA430746 | 3 | 0 | 29 | |
| 2292390 | AI648504 | AI648504 | 1 | 0 | 1 | |
| 1985408 | AI254597 | AI254597 | 1 | 0 | 0 | |
| 2251282 | AI658484 | AA363270 | 1 | 0 | 1 | |
| 1373633 | AA837669 | AA837669 | 1 | 0 | 0 | |
| 2122708 | AI521444 | AI521444 | 1 | 0 | 33 | |
| 753401 | AA406401 | T29223 | 1 | 0 | 13 | |
| 2216215 | AI654529 | AI654529 | 1 | 0 | 0 | |
| 810889 | AA459288 | AA454869 | 5 | 3 | 76 | |
| 811573 | AA458520 | AA458520 | 1 | 0 | 1 | |
| 2216412 | AI654749 | AA464522 | 3 | 0 | 12 | |
| 754929 | AA422036 | AA422036 | 2 | 0 | 0 | |
| 981382 | AA525940 | AA525940 | 1 | 0 | 0 | |
| 1965573 | AI364874 | AI364874 | 1 | 0 | 11 | |
| 981208 | AA526408 | AA526408 | 1 | 0 | 0 | |
| 757030 | AA429021 | AA402679 | 24 | 12 | 79 | |
| 742056 | AA405746 | AA405746 | 2 | 0 | 25 | |
| 1861875 | AI053808 | AI053808 | 1 | 0 | 17 | |
| 1373857 | AA828779 | AA482061 | 2 | 0 | 0 | |
| 1373888 | AA828792 | AA828792 | 1 | 0 | 0 | |
| 810216 | AA463996 | AA402679 | 24 | 12 | 79 | |
| 1374124 | AA828604 | AA828604 | 1 | 0 | 0 | |
| 981025 | AA526011 | AA526011 | 1 | 0 | 0 | |
| 1212476 | AA643476 | AA643450 | 4 | 0 | 8 | |
| 800175 | AA581095 | AA581095 | 1 | 0 | 0 | |
| 741705 | AA402081 | AA402081 | 1 | 0 | 0 | |
| 740320 | AA477801 | AA482651 | 2 | 0 | 6 | |
| 756537 | AA436442 | AA292747 | 2 | 0 | 2 | |
| 897358 | AA496757 | AA363048 | 1 | 0 | 11 | |
| 1965502 | AI364842 | AI364842 | 1 | 0 | 0 | |
| 755590 | AA419204 | AA419204 | 1 | 0 | 0 | |
| 1964045 | AI280624 | AI280624 | 1 | 0 | 0 | |
| 1965524 | AI364851 | AI364851 | 1 | 0 | 0 | |
| 740416 | AA477828 | AA422036 | 2 | 0 | 0 | |
| 770267 | AA434256 | AA434256 | 1 | 0 | 0 | |
| 811005 | AA485364 | AA292026 | 2 | 0 | 20 | |
| 741183 | AA402548 | AA402548 | 1 | 0 | 4 | |
| 727859 | AA393566 | AA362742 | 1 | 0 | 34 | |

### Example 2

### Analysis of cDNA Expression Using Microarray Analysis and Real Time PCR

The electronic subtraction performed in Example 1 identified 10,862 sequences. Of the original sequences, 1688 were excluded from continued analysis due to duplication of the sequences. The remaining 9174 sequences were evaluated for their expression profiles in a variety of tumor and normal tissue types using microarray analysis.

Using this approach, the cDNA sequences were PCR amplified using vector specific primers. The resulting PCR products were then arrayed randomly onto one of two chips. Ovary Chip #3 contained 2784 EST clones and Ovary Chip #4 contained 4882 clones.

Their mRNA profiles in tumor and normal tissues were then examined using microarray technology essentially as described (Shena M. et al., 1995 Science 270:467-470). The probes were selected from two groups, one of which is fluorescently labeled with Cy3 (Group I, typically tumor tissues, see Table I) and the other with Cy5 (Group II, typically normal tissue [N], see Table 3). The Chips were then hybridized with probe pairs (one from Group I and one from Group II) and the fluorescent intensities measured. The mean fluorescent intensity of probes from Group I was then compared to the mean fluorescence of probes from Group II. A ratio was then determined using computational analysis. This ratio determines the relevance of a particular sequence to different tissues, both normal and tumor. In addition to this ratio, sequences were also classified based on the level of background present in Group II. If the mean fluorescent signal was <0.1, this identified sequences with low background, between 0.1-0.2 identified sequences with some background, and <0.2 identified sequences with potentially high background.

Ovary Chip #3 was probed with 35 different probe pairs (see Table 3, Groups I and II for details). Two different types of analysis were then performed:

1. The mean fluorescent intensity of ovary tumor tissue compared to the mean fluorescent intensity of normal tissue (minus ovary). Sequences were selected using this analysis if the ratio was greater than three and the background <0.2.

2. The mean fluorescent intensity of ovary tumor and ovary normal tissue compared to the mean fluorescent intensity of essential normal tissue (minus ovary). Sequences were selected using this analysis if the ratio was greater than 2.5 and the background <0.2.

TABLE 3
Probes Used to Analyze Ovary Chip #3

| Cy3 Probe | | Cy5 Probe | |
|---|---|---|---|
| Ovary Tumor (IIIC) | 261A | SPACT37 | Adrenal Gland (essential) |
| Ovary Tumor (IIIC) | 264A | 396A | Skin N (essential) |
| Ovary Tumor (IIIC) | 265A | SPACT56 | Thymus N (essential) |
| Ovary Tumor (IIIC) | 288A | 600C | Bronchus N (essential) |
| Papillary Serous Cystic Neo. | 854A | 785B | Bronchus N (essential) |
| (IA) | | | |
| Papillary Serous (IA) | 855A | 407B | Bone N |
| Papillary Serous (IA) | 856A | 484A | Peritoneum Epithelial N |
| | | | (essential) |
| Papillary Serous Adeno (IA) | 603A | SPACT52 | Pituitary Gland N (essential) |
| Papillary Serous (IB) | 857A | SPACT40 | Skeletal muscle N (essential) |
| Papillary Serous Adeno (IB) | 385A | SPACT55 | Stomach (essential) |
| Papillary Serous Neo (IC) | 492A | SPACT54 | Spleen N (essential) |
| Papillary Serous (IIA) | 858A | 862A | Pancreas N (essential) |
| Papillary Serous (IIB) | 859A | S27 | Ovary N |
| Serous Borderline Tumor | 605A | SPACT45 | Spinal cord N (essential) |
| (IIIC) | | | |
| Papillary Serous Carcinoma | 495A | SPAAm1 | Heart N |
| (III) | | | |
| Mucinous Cystadeno (IB) | 381C | S7 | Ovary N |
| Mucinous Adeno (IIIC) | 382A | 5449A | Ovary N |
| Mets Mucinous Adeno (IIIC) | 428B | SPACT53 | Small intestine N (essential) |
| Endometrioid Adeno. (IA) | 491A | 502B | Esophagus N (essential) |
| Endometrioid Adeno. (II) | 335A | 199A | Colon N (essential) |
| Endometrioid Adeno. (II-III) | 494A | SPACT46 | Thyroid gland N (essential) |
| Cystic Endometrioid. (IIIC) | 860A | 783A | PBMC (resting) (essential) |
| Clear cell adeno. (I) | 604A | 415A | Aorta N (essential) |
| Clear cell (IA) | 607A | 776A | Trachea N (essential) |
| Granulosa Cell Tumor (IA) | S25 | CT25 | Trachea N (essential) |
| Granulosa Cell Tumor (IA) | S22 | PAN2000 | Pancreas N (pool) (essential) |
| Germ Cell Tumor | 386A | S92 | Breast (HMEC) N (essential) |
| Papillary Serous Adeno. (IIIB) | 602A | 328B/C | Bladder N (essential) |
| Papillary Serous Adeno. (IIIC) | S23 | SPACT49 | Bone marrow N (essential) |
| Papillary Serous Cystadeno. | 606A | SPAAm2 | Lung N (essential) |
| (IIIB) | | | |
| Mets Papillary Adeno. (IIIA) | 383A | 302B | Kidney N (essential) |
| Mets Papillary Serous. (IIIB) | 384A | S44/782A | PBMC (activated) (essential) |
| Mets. Pap. Serous Cystadeno | 426A | 603A | Ovary tumor (Cy3 match) |
| (IIIC) | | | |
| Mets Pap. Adeno (IIIC) | 429A | 270B | Liver N (essential) |
| Ovary Tumor | 427A | SPACT50 | Brain N (essential) |

Microarray analysis of clones that showed significant expression levels in tumor samples when compared to normals is presented in Table 4.

TABLE 4
Microarray Analysis From Ovary Chip #3

| | | | | | |
|---|---|---|---|---|---|
| Clone ID | | | Mean | Mean | Candidate |
| IMAGE # | Accession # | Ratio | Signal 1 | Signal 2 | Name |

Threshold >3.0; Mean signal 2 <0.1; Ranked by Mean signal 1.

| | | | | | |
|---|---|---|---|---|---|
| 595243 | AA164216/AA164215 | 6.67 | 0.632 | 0.095 | O1028C |
| 594484 | AA180009/AA164745 | 5.6 | 0.432 | 0.077 | O1029C |
| 843028 | AA488541/AA488406 | 3.85 | 0.368 | 0.096 | O1030C-R |
| 76058 | T59567/T59521 | 3.07 | 0.278 | 0.09 | O1031C |
| 544863 | AA075131/AA075149 | 3.24 | 0.248 | 0.077 | |
| 741139 | AA402754/AA402207 | 3.3 | 0.225 | 0.068 | |
| 545242 | AA076182/AA076085 | 3.23 | 0.225 | 0.07 | |
| 75673 | T58484 | 3.18 | 0.189 | 0.059 | |
| 811600 | AA458533/AA454609 | 3.95 | 0.183 | 0.046 | O1032C |
| 739457 | AA477250/AA477249 | 3.26 | 0.183 | 0.056 | |
| 77262 | T50256/T50206 | 3.12 | 0.175 | 0.056 | |
| 77257 | T50212 | 3.06 | 0.172 | 0.056 | |
| 74768 | T47079 | 3.02 | 0.17 | 0.056 | |
| 593444 | AI733904/AI732611/AA160259/ | 4.96 | 0.164 | 0.033 | |
| | AA160258 | | | | |
| 811063 | AI734202/AI732823/AA485619/ | 3.03 | 0.164 | 0.054 | |
| | AA485451 | | | | |
| 593218 | AI733902/AI732609/AA159613/ | 3.01 | 0.149 | 0.05 | |
| | AA159743 | | | | |
| 595232 | AI732624/AA173518 | 4.19 | 0.144 | 0.034 | |
| 75759 | T58522 | 3.68 | 0.142 | 0.038 | |
| 714232 | AA293601 | 3.03 | 0.134 | 0.044 | |
| 809858 | AA464305/AA455119 | 3.56 | 0.129 | 0.036 | |
| 489076 | AA057195/AA057129 | 3.2 | 0.129 | 0.04 | |
| 771328 | AA476222/AA476223 | 3.23 | 0.111 | 0.034 | |
| 755093 | AI821682/AI820932/AA482660/ | 5.95 | 0.094 | 0.016 | |
| | AA482508 | | | | |
| 771053 | AA430718/AA427528 | 3.37 | 0.086 | 0.025 | |
| 77250 | T50141 | 3.22 | 0.082 | 0.025 | |
| 810109 | AA465048/AA464979 | 4.65 | 0.078 | 0.017 | |
| 136984 | AF074992/R35849 | 4.31 | 0.068 | 0.016 | |
| 809776 | AA454784/AA454732 | 6.89 | 0.028 | 0.004 | |

Threshold >3.0; Signal 2 <0.2; Ranked by mean Signal 1.

| | | | | | |
|---|---|---|---|---|---|
| 593439 | AA165668/AA165632 | 9.75 | 1.149 | 0.118 | O1033C |
| 76081 | T59590 | 3.86 | 0.708 | 0.183 | |
| 724886 | AA404613/AA291468 | 3.04 | 0.532 | 0.175 | |
| 724962 | AA404631/AA291495 | 3.36 | 0.438 | 0.13 | HPP14 |
| 283151 | N45230 | 3.2 | 0.379 | 0.119 | |
| 595363 | AA164920/AA164919 | 3.04 | 0.31 | 0.102 | |

Threshold >3.0; Mean Signal 2 >0.2; Ranked by Mean Signal 1.

| | | | | | |
|---|---|---|---|---|---|
| 544548 | AA075105/AA074952 | 4.23 | 1.768 | 0.418 | |
| 595220 | AA164851/AA164850 | 3.31 | 1.598 | 0.482 | |
| 545443 | AA079191/AA079190 | 3.29 | 1.241 | 0.377 | |
| 545185 | AA076101 | 3.04 | 1.152 | 0.379 | |
| 714080 | AA284545/AA284815 | 5.09 | 1.05 | 0.206 | |
| 595449 | AA173739/AA173383 | 4.95 | 0.999 | 0.202 | O1034C |

Threshold >2.5; Mean Signal 2 <0.1; Ranked by Mean Signal 1.

| | | | | |
|---|---|---|---|---|
| AA176693/AA173996 | 15.64 | 1.43 | 0.091 | |
| AI821564/AA284659/ | 4.71 | 0.323 | 0.069 | |
| AA284658 | | | | |
| AA075034/AA075033 | 3.01 | 0.291 | 0.097 | O1035C |
| AA394090/AA293773 | 3.04 | 0.275 | 0.091 | O1036C |
| T57605 | 2.92 | 0.19 | 0.065 | |
| AA456327/AA454681 | 3.19 | 0.188 | 0.059 | |
| AA164782/AA164781 | 2.85 | 0.138 | 0.049 | |
| AA166898/AA166756 | 2.73 | 0.129 | 0.047 | |
| AA075203/AA075202 | 3.35 | 0.128 | 0.038 | |
| AA405395/AA402883 | 3.07 | 0.126 | 0.041 | |
| AA172236/AA172056 | 3.17 | 0.124 | 0.039 | |
| T57611/T57557 | 2.62 | 0.118 | 0.045 | |
| AA419214/AA419229 | 2.68 | 0.112 | 0.042 | |
| AA171974/AA171755 | 2.89 | 0.109 | 0.038 | |
| AA076270/AA076269 | 5.32 | 0.087 | 0.016 | |
| AA464689 | 2.61 | 0.074 | 0.028 | |
| AA456446/AA454554 | 2.57 | 0.065 | 0.025 | |
| T60090 | 3.66 | 0.045 | 0.012 | |
| AA075211/AA075126 | 3.91 | 0.036 | 0.009 | |

Threshold >2.5; Mean Signal 2 >2.0; Ranked by Mean Signal 1.

| | | | |
|---|---|---|---|
| AA076228/AA076227 | 5.02 | 0.812 | 0.162 |
| AA075310 | 4.98 | 0.582 | 0.117 |
| T58551/T58501 | 3.15 | 0.528 | 0.167 |
| T59551 | 3.13 | 0.431 | 0.138 |
| AA464691 | 2.62 | 0.382 | 0.146 |
| T52869/T52868 | 3.16 | 0.37 | 0.117 |
| AF147380/T59906/T59850 | 2.55 | 0.335 | 0.131 |

Threshold >2.5; Mean Signal 2 <0.1; Ranked by Mean Signal 1.

| | | | | |
|---|---|---|---|---|
| | AA172293/AA171844 | 2.82 | 0.257 | 0.091 |
| | AA446928/AA443351 | 2.55 | 0.164 | 0.064 |
| | AA164691 | 3.09 | 0.107 | 0.035 |
| | AA160156/AA160155 | 2.68 | 0.088 | 0.033 |
| | AI732624/AA173518/AA173428 | 3.08 | 0.086 | 0.028 |
| 595232 | AI732624/AA173518 | 4.19 | 0.144 | 0.034 |
| | AA411128/AA292424 | 2.74 | 0.078 | 0.028 |
| | AI734109/AI732705/AA428413/ | 2.55 | 0.072 | 0.028 |
| | AA427400 | | | |

Of those sequences presented in Table 4, several were selected for Real Time PCR analysis of an extensive panel of normal and tumor tissues. Real Time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, this was done by extracting mRNA from tumor and normal tissue and preparing cDNA using standard techniques. Real Time PCR was then performed using a Perkin-Elmer/Applied Biosystems (Foster City, Calif.) 7700 Prism instrument. Matching primers and fluorescent probes were then designed for each of the 6 sequences and optimal concentrations for amplification and detection determined. The cDNA content was standardized using PCR specific for β-Actin. The amount of specific RNA was then determined by either comparing the number of copies present per 1000 picograms of β-Actin or by setting a baseline using the sample, which showed the lowest level of the gene of interest.

Table 5 contains the Real Time PCR results for 6 samples selected from Chip #3.

TABLE 5
Real Time PCR Expression
Profiles of Sequences Identified on Ovary Chip #3

| | | SEQ ID | Ovary Tumor Tissue and | |
|---|---|---|---|---|
| Clone ID | Image # | NO | tumor associated tissue | Normal Tissue |
| 1029C | 94484 | 1928; | HOE in 11/23; OE in 3/23; E | OE in 1/3 Ovary |
| | | 10,864 | 3/23; E in 2/2 SCID grown | |
| | | | tumors; E in 1/1 tumor cell lines | |
| 1030C | 843028 | 10,196; | HOE in 5/20; OE in 5/20; E in | E in ovary |
| | | 10,868; | 9/20 | |
| | | 10,870 | | |
| 1032C | 811600 | 5085; | HOE in 1/22; OE in 1/22; E in | OE in Bronchia |
| | | 10,872; | 14/22. | |
| | | 10,873 | | |
| 1033C | 593439 | 10,874; | HOE in 4/21; OE in 4/21; E | No expression |
| | | 10,875 | 10/21 | detected |
| 1034C | 595449 | 2409; | HOE in 4/22; OE in 7/22; E in | E in brain, brain |
| | | 10,876 | 7/22; OE in 1/2 SCID grown | pool, pancreas, |
| | | | tumors. | |
| O1036C | 726384 | 3228; | HOE in 1/13; OE in 4/13; E in | E in PBMC |
| | | 3239; | 8/13; E in 2/2 SCID derived | (activated), Skeletal |
| | | 10,880; | tissue. | muscle, spleen, bone |
| | | 10,881; | | marrrow, bronchia, |
| | | 10,882; | | brain |
| | | 10883; | | |
| | | 10,884 | | |

HOE = highly over expressed; OE = over expressed; E = expressed.

Ovary Chip #4 contained 4882 clones from the electronic subtraction process described in Example 1. This Chip was probed with 35 different probe paris (see Table 6, Groups I and II for details). Two different types of analysis were then performed:

1. The mean fluorescent intensity of ovary tumor tissue compared to the mean fluorescent intensity of normal tissue (minus ovary). Sequences were selected using this analysis if the ratio was greater than three and the background <0.2.

2. The mean fluorescent intensity of ovary tumor and ovary normal tissue compared to the mean fluorescent intensity of essential normal tissue (minus ovary). Sequences were selected using this analysis if the ratio was greater than 2.5 and the background <0.2.

TABLE 6
Probes Used to Analyze Ovary Chip #4

| Group I: Cy3 Probe | | Group II: Cy5 Probe | |
|---|---|---|---|
| Ovary Tumor (IIIC) | 201 | SPACT84 | Adrenal Gland N |
| Ovary Tumor (IIIC) | 264A | SK2000 | Skin Pool N |
| Ovary Tumor (IIIC) | 263A | SPACT70 | Thymus N |
| Ovary Tumor (IIIC) | 288A | 785A/D | Bronchus N |
| Papillary Serous adeno. (IA) | 1068A | 784B | Bronchus N |
| Serous Cystadenoma (IB) | 1084A | 741A/B | Bone N |
| Papillary Serous (IA) | 856A | BP2000 | Breast Pool N |
| Papillary Serous Adeno. (IA) | 603A | SPACT67 | Pituitary Gland N |
| Papillary Serous Cystadeno. | 857A | SPACT75 | Skeletal |
| (IB) | | | Muscle N |
| Papillary Serous Adeno. (IB) | 385A | SPACT66 | Stomach N |
| Papillary Serous Neo. (IC) | 492A | SPACT69 | Spleen N |
| Papillary Serous Cystadeno. | 858A | 862A | Pancreas N |
| IIA | | | |
| Mucious Carcinoma (IIA) | 1130A | S27 | Ovary N |
| Mets. Papillary Serous (III) | 495A | SPACT73 | Heart N |
| Mets. Pap. Serous Cystadeno. | 606A | S52/ | Lung N |
| (IIIB) | | SPACT78 | |
| Papillary Serous Adeno. (IIIB) | 602A | 328B/C | Bladder N |
| Serous Borderline Tumor | 605A | SPACT59 | Spinal Cord N |
| (IIIC) | | | |
| Papillary Serous Adeno. (IIIC) | S23 | SPACT49 | Bone Marrow N |
| Mets. Papillary Adeno. (IIIA) | 383A | KP2000 | Kidney Pool N |
| Mets. Adenocarcinoma (IIIC) | 1069A | 1155A | PBMC (activated) |
| Mets. Papillary Adeno. (IIIC) | 1070A | 1070A | Ovary Tumor |
| | | | Cy3 match |
| Mets. Papillary Adeno. (III) | 429A | SPACT81 | Liver N |
| Mets. Pap. Serous Adeno. | 427A | SPACT85 | Brain N |
| (IIIC) | | | |
| Mucinous Cystadeno. (IB) | 381C | S7 | Ovary N |
| Mets. Endometrial Adeno. | 1127A | S449A | Ovary N |
| (III) | | | |
| Mets. Mucinous Adeno. (IIIC) | 428B | SPACT86 | Small Intestine N |
| Endometrioid Carcinoma (IA) | 1071A | 502B | Esophagus N |
| Endometrioid Adeno. (IA) | 1081A | 1004A | Colon N |
| Mets. Adeno. (II-III) | 494A | 886A | Thyroid Gland N |
| Cystic Edometrioid Adeno. | 860A | 783A/ | PBMC Pool |
| (IIIC) | | 888A | (resting) |
| Clear Cell Adeno. (IA) | 604A | 415C | Aorta N |
| Clear Cell/Endometrioid (IA) | 607A | CT25 | Trachea Pool N |
| Granulosa Cell Tumor (IA) | S25 | 772B | Trachea N |
| Granulosa Cell Tumor (IA) | 1082A | SPACT58 | Pancreas Pool N |
| Ovary Germ-Cell Tumor (I) | 386A | S12 | Mammary |
| | | | Epithelial N |

Microarray analysis of clones that showed significant expression levels in tumor samples when compared to normals is presented in Table 7.

TABLE 7
Microarray Analysis From Ovary Chip #4

| | | | | | |
|---|---|---|---|---|---|
| Clone ID | | | Mean | Mean | Candidate |
| IMAGE # | Accession # | Ratio | Signal 1 | Signal 2 | Name |

Threshold >2.0; Mean Signal 2 <0.1; Mean Signal 1 >0.2.

| | | | | | |
|---|---|---|---|---|---|
| | | 6.67 | 0.358 | 0.054 | O1033C |
| | | 4.82 | 0.247 | 0.051 | O1029C |
| | | 4.59 | 0.22 | 0.048 | O1034C |
| | | 3.32 | 0.209 | 0.063 | Phospholipase a2 |
| 2216507 | AI653489 | 3.26 | 0.254 | 0.078 | |
| 2292394 | AI648506 | 2.94 | 0.235 | 0.08 | |
| 2216151 | AI654462 | 2.64 | 0.202 | 0.076 | |
| 981215 | AA526423 | 2.56 | 0.227 | 0.089 | |
| 1373880 | AA828788 | 2.16 | 0.201 | 0.093 | |
| 1373112 | AA837621 | 2.1 | 0.201 | 0.096 | |

Threshold >2.0; Mean Signal 2 <0.1; Mean Signal 1 = 0.1-0.2.

| | | | | | |
|---|---|---|---|---|---|
| 2002493 | AI224233 | 15.14 | 0.102 | 0.007 | |
| 1373281 | AA827974 | 4.19 | 0.102 | 0.024 | |
| 2216154 | AI654454 | 3.52 | 0.195 | 0.055 | |
| 755378 | AA410595 | 3.15 | 0.107 | 0.034 | |
| 769666 | AA428329 | 2.97 | 0.112 | 0.038 | |
| 2216636 | AI653555 | 2.92 | 0.123 | 0.042 | |
| 1455124 | AA909822 | 2.89 | 0.183 | 0.063 | |
| 1374123 | AA828611 | 2.86 | 0.176 | 0.061 | |
| 770437 | AA427513 | 2.79 | 0.154 | 0.055 | |
| 739389 | AA476860 | 2.78 | 0.153 | 0.055 | |
| 1985442 | AI254625 | 2.72 | 0.111 | 0.041 | |
| 981039 | AA526017 | 2.67 | 0.139 | 0.052 | |
| 755111 | AA411372 | 2.66 | 0.11 | 0.042 | |
| 1965439 | AI356599 | 2.65 | 0.136 | 0.051 | |
| 755436 | AA419044 | 2.63 | 0.138 | 0.053 | |
| 742144 | AA405975 | 2.58 | 0.104 | 0.04 | |
| 1373104 | AA837606 | 2.56 | 0.172 | 0.067 | |
| 1373878 | AA828778 | 2.55 | 0.156 | 0.061 | |
| 1454766 | AA908470 | 2.55 | 0.152 | 0.06 | |
| 1373719 | AA828882 | 2.41 | 0.163 | 0.068 | |
| 1374009 | AA828703 | 2.36 | 0.184 | 0.078 | |
| 1373380 | AA828138 | 2.34 | 0.17 | 0.073 | |
| 1373381 | AA828148 | 2.32 | 0.161 | 0.07 | |
| 1964546 | AI284862 | 2.29 | 0.1 | 0.044 | |
| 1192366 | AA650614 | 2.27 | 0.157 | 0.069 | |
| 2260607 | AI590617 | 2.25 | 0.102 | 0.045 | |
| 1154834 | AA642166 | 2.25 | 0.155 | 0.069 | |
| 981113 | AA526205 | 2.23 | 0.111 | 0.05 | |
| 2292558 | AI648453 | 2.22 | 0.1 | 0.045 | |
| 981376 | AA525937 | 2.21 | 0.138 | 0.063 | |
| 1374125 | AA828612 | 2.21 | 0.159 | 0.072 | |
| 1374171 | AA828647 | 2.21 | 0.183 | 0.082 | |
| 1965552 | AI364864 | 2.21 | 0.142 | 0.064 | |
| 981310 | AA525800 | 2.2 | 0.109 | 0.05 | |
| 1373500 | AA847986 | 2.18 | 0.181 | 0.083 | |
| 1373375 | AA828135 | 2.17 | 0.173 | 0.08 | |
| 1190617 | AA650078 | 2.17 | 0.155 | 0.071 | |
| 2292555 | AI648459 | 2.16 | 0.12 | 0.056 | |
| 755357 | AA410276 | 2.15 | 0.153 | 0.071 | |
| 1373287 | AA827977 | 2.15 | 0.166 | 0.077 | |
| 1192390 | AA650618 | 2.14 | 0.182 | 0.085 | |
| 2260515 | AI590596 | 2.13 | 0.18 | 0.085 | |
| 981372 | AA525935 | 2.12 | 0.189 | 0.089 | |
| 980932 | AA525774 | 2.11 | 0.199 | 0.094 | |
| 1374020 | AA828698 | 2.11 | 0.178 | 0.085 | |
| 1373311 | AA828039 | 2.09 | 0.157 | 0.075 | |
| 1047907 | AA773935 | 2.09 | 0.158 | 0.076 | |
| 1154937 | AA627343 | 2.09 | 0.197 | 0.094 | |
| 1373504 | AA847988 | 2.08 | 0.19 | 0.091 | |
| 1963836 | AI280530 | 2.08 | 0.187 | 0.09 | |
| 981337 | AA525918 | 2.08 | 0.193 | 0.093 | |
| 1373879 | AA828787 | 2.08 | 0.182 | 0.088 | |
| 980966 | AA525874 | 2.07 | 0.199 | 0.096 | |
| 2292391 | AI648510 | 2.07 | 0.115 | 0.056 | |
| 2292584 | AI648468 | 2.06 | 0.104 | 0.051 | |
| 981068 | AA526095 | 2.06 | 0.176 | 0.086 | |
| 1373285 | AA827976 | 2.06 | 0.165 | 0.08 | |
| 1373419 | AA826312 | 2.06 | 0.153 | 0.074 | |
| 1374172 | AA828640 | 2.06 | 0.161 | 0.078 | |
| 1373400 | AA826292 | 2.04 | 0.162 | 0.079 | |
| OVM-84 | | 2.04 | 0.166 | 0.081 | OVM-84 |
| 1373695 | AA828597 | 2.03 | 0.15 | 0.074 | |
| 1373807 | AA828690 | 2.03 | 0.118 | 0.058 | |
| 1373415 | AA826310 | 2.03 | 0.146 | 0.072 | |
| 981359 | AA525929 | 2.03 | 0.172 | 0.084 | |
| 1373309 | AA828038 | 2.03 | 0.16 | 0.079 | |
| 2260575 | AI591141 | 2.02 | 0.107 | 0.053 | |
| 755715 | AA496499 | 2.02 | 0.165 | 0.082 | |
| 809959 | AA454835 | 2.02 | 0.152 | 0.075 | |
| 1373509 | AA848000 | 2.01 | 0.193 | 0.096 | |
| 1374033 | AA829114 | 2.01 | 0.172 | 0.086 | |
| 1373080 | AA837588 | 2.01 | 0.187 | 0.093 | |
| 1373691 | AA828596 | 2.01 | 0.193 | 0.096 | |
| 1964868 | AI355523 | 2.01 | 0.109 | 0.054 | |
| 1190662 | AA650084 | 2.01 | 0.179 | 0.089 | |
| 1154908 | AA627330 | 2 | 0.178 | 0.089 | |

Threshold >2.0; Mean Signal 2 <0.1; Mean Signal 1 <0.1.

| | | | | | |
|---|---|---|---|---|---|
| 1985131 | AI242020 | 9.05 | 0.028 | 0.003 | |
| 1985090 | AI241990 | 8.43 | 0.024 | 0.003 | |
| 1985420 | AI254602 | 7.47 | 0.029 | 0.004 | |
| 1985633 | AI254657 | 7.34 | 0.029 | 0.004 | |
| 1985494 | AI254938 | 6.94 | 0.065 | 0.009 | |
| 1985584 | AI251343 | 6.81 | 0.056 | 0.008 | |
| 1985071 | AI241981 | 6.49 | 0.026 | 0.004 | |
| 1985548 | AI251317 | 6.36 | 0.03 | 0.005 | |
| 1985096 | AI241992 | 6.04 | 0.027 | 0.005 | |
| 1985539 | AI251255 | 5.86 | 0.053 | 0.009 | |
| 1985503 | AI254963 | 5.69 | 0.075 | 0.013 | |
| 1985111 | AI242006 | 5.65 | 0.022 | 0.004 | |
| 1985085 | AI241987 | 5.65 | 0.023 | 0.004 | |
| 810330 | AA464144 | 5.52 | 0.018 | 0.003 | |
| 980920 | AA525758 | 5.34 | 0.067 | 0.012 | |
| 1985162 | AI254100 | 5.2 | 0.028 | 0.005 | |
| 930200 | AA502530 | 5.17 | 0.035 | 0.007 | |
| 1985535 | AI251253 | 5.09 | 0.044 | 0.009 | |
| 1985593 | AI251369 | 5.07 | 0.06 | 0.012 | |
| 2003171 | AI250559 | 4.92 | 0.026 | 0.005 | |
| 810462 | AA457130 | 4.84 | 0.025 | 0.005 | |
| 1985079 | AI241985 | 4.54 | 0.038 | 0.008 | |
| 769684 | AA428757 | 4.42 | 0.019 | 0.004 | |
| 1985127 | AI242019 | 4.42 | 0.031 | 0.007 | |
| 1985630 | AI254644 | 4.41 | 0.059 | 0.013 | |
| 1985576 | AI251339 | 4.37 | 0.042 | 0.01 | |
| 1985558 | AI251322 | 4.36 | 0.074 | 0.017 | |
| 1984612 | AI255127 | 4.35 | 0.04 | 0.009 | |
| 1373305 | AA828036 | 4.32 | 0.08 | 0.018 | |
| 2002838 | AI224248 | 4.06 | 0.015 | 0.004 | |
| 1984571 | AI251722 | 3.92 | 0.028 | 0.007 | |
| 1985520 | AI251235 | 3.78 | 0.045 | 0.012 | |
| 1985272 | AI254763 | 3.75 | 0.038 | 0.01 | |
| 2002656 | AI223675 | 3.62 | 0.022 | 0.006 | |
| 1985594 | AI251359 | 3.6 | 0.039 | 0.011 | |
| 1985474 | AI254929 | 3.47 | 0.053 | 0.015 | |
| 2119169 | AI401777 | 3.11 | 0.07 | 0.023 | |
| 755480 | AA410639 | 3.02 | 0.048 | 0.016 | |
| 1985049 | AI251638 | 2.96 | 0.047 | 0.016 | |
| 2006525 | AI265778 | 2.91 | 0.056 | 0.019 | |
| 1984955 | AI252857 | 2.87 | 0.049 | 0.017 | |
| 810467 | AA457141 | 2.83 | 0.071 | 0.025 | |
| 993046 | AA570694 | 2.83 | 0.091 | 0.032 | |
| 810530 | AA464548 | 2.77 | 0.018 | 0.007 | |
| 1985621 | AI254651 | 2.73 | 0.037 | 0.014 | |
| 2002637 | AI223666 | 2.71 | 0.027 | 0.01 | |
| 755865 | AA496479 | 2.67 | 0.073 | 0.027 | |
| 1985448 | AI254906 | 2.63 | 0.015 | 0.006 | |
| 2292434 | AI648563 | 2.63 | 0.067 | 0.026 | |
| 810498 | AA457154 | 2.6 | 0.022 | 0.009 | |
| 2003068 | AI223766 | 2.56 | 0.041 | 0.016 | |
| 2006294 | AI279388 | 2.54 | 0.055 | 0.022 | |
| 1985556 | AI251321 | 2.53 | 0.053 | 0.021 | |
| 1985494 | AI254938 | 2.53 | 0.058 | 0.023 | |
| 1985040 | AI251623 | 2.52 | 0.026 | 0.01 | |
| 1984903 | AI254586 | 2.5 | 0.02 | 0.008 | |
| 2292460 | AI648530 | 2.5 | 0.07 | 0.028 | |
| 1984941 | AI252841 | 2.5 | 0.034 | 0.014 | |
| 770747 | AA454520 | 2.48 | 0.045 | 0.018 | |
| 1985061 | AI251642 | 2.48 | 0.023 | 0.009 | |
| 2260925 | AI591032 | 2.48 | 0.085 | 0.034 | |
| 2006359 | AI264852 | 2.47 | 0.059 | 0.024 | |
| 2292490 | AI648545 | 2.43 | 0.082 | 0.034 | |
| 770522 | AA456762 | 2.42 | 0.052 | 0.022 | |
| 2292472 | AI648533 | 2.4 | 0.074 | 0.031 | |
| | | 2.35 | 0.092 | 0.039 | O1031C |
| 2002584 | AI223506 | 2.34 | 0.024 | 0.01 | |
| 2006508 | AI265914 | 2.34 | 0.038 | 0.016 | |
| 2002702 | AI223706 | 2.34 | 0.041 | 0.018 | |
| 2002948 | AI223609 | 2.31 | 0.028 | 0.012 | |
| 2002540 | AI223470 | 2.31 | 0.027 | 0.012 | |
| 2260893 | AI591004 | 2.3 | 0.082 | 0.036 | |
| 2003136 | AI250523 | 2.27 | 0.023 | 0.01 | |
| 2260680 | AI590757 | 2.26 | 0.056 | 0.025 | |
| 2219675 | AI738841 | 2.23 | 0.067 | 0.03 | |
| 2006285 | AI279392 | 2.22 | 0.033 | 0.015 | |
| 1984994 | AI252969 | 2.18 | 0.041 | 0.019 | |
| 2260809 | AI608710 | 2.17 | 0.056 | 0.026 | |
| 2260492 | AI590577 | 2.15 | 0.081 | 0.038 | |
| 1964644 | AI287912 | 2.14 | 0.098 | 0.046 | |
| 770961 | AA428171 | 2.13 | 0.032 | 0.015 | |
| 810067 | AA455305 | 2.12 | 0.099 | 0.047 | |
| 2107496 | AI380369 | 2.12 | 0.06 | 0.028 | |
| 2292567 | AI648462 | 2.12 | 0.085 | 0.04 | |
| 2260591 | AI590610 | 2.11 | 0.088 | 0.042 | |
| 2261022 | AI591055 | 2.11 | 0.077 | 0.036 | |
| 2292702 | AI612846 | 2.11 | 0.088 | 0.042 | |
| 2002858 | AI224270 | 2.09 | 0.04 | 0.019 | |
| 2006307 | AI279408 | 2.08 | 0.058 | 0.028 | |
| 1984931 | AI252836 | 2.08 | 0.035 | 0.017 | |
| 2260917 | AI591022 | 2.08 | 0.083 | 0.04 | |
| 770927 | AA433865 | 2.07 | 0.024 | 0.011 | |
| 2006292 | AI279387 | 2.07 | 0.036 | 0.017 | |
| 2261010 | AI609587 | 2.07 | 0.084 | 0.041 | |
| 741039 | AA402296 | 2.06 | 0.088 | 0.043 | |
| 2006364 | AI264845 | 2.06 | 0.013 | 0.006 | |
| 1985021 | AI251613 | 2.04 | 0.024 | 0.012 | |
| 1374360 | AA828959 | 2.04 | 0.034 | 0.017 | |
| 1985005 | AI252985 | 2.04 | 0.046 | 0.022 | |
| 2258189 | AI623135 | 2.03 | 0.097 | 0.047 | |
| 2006320 | AI279404 | 2.03 | 0.017 | 0.008 | |
| 770715 | AA454497 | 2.02 | 0.028 | 0.014 | |
| 2261186 | AI609337 | 2.02 | 0.064 | 0.032 | |
| 2216400 | AI654744 | 2.02 | 0.09 | 0.045 | |
| 2006496 | AI265908 | 2.02 | 0.049 | 0.024 | |
| 2292705 | AI612857 | 2.02 | 0.089 | 0.044 | |
| 1964750 | AI283331 | 2.01 | 0.091 | 0.045 | |
| 2260699 | AI590770 | 2.01 | 0.062 | 0.031 | |
| 1984950 | AI252845 | 2.01 | 0.045 | 0.023 | |
| 770978 | AA428247 | 2 | 0.096 | 0.048 | |
| 770998 | AA428253 | 2 | 0.097 | 0.048 | |
| 2292390 | AI648504 | 2 | 0.083 | 0.041 | |
| 1985408 | AI254597 | 2 | 0.045 | 0.022 | |
| 2251282 | AI658484 | 2 | 0.099 | 0.049 | |

Threshold >2.0; Mean Signal 2 = 0.1-0.2; Ranked by Ratio

| | | | | | |
|---|---|---|---|---|---|
| 1373633 | AA837669 | 4.24 | 0.496 | 0.117 | |
| 2122708 | AI521444 | 2.96 | 0.397 | 0.134 | |
| 753401 | AA406401 | 2.36 | 0.266 | 0.113 | |
| 2216215 | AI654529 | 2.35 | 0.336 | 0.143 | |
| 810889 | AA459288 | 2.25 | 0.316 | 0.14 | |
| 811573 | AA458520 | 2.19 | 0.224 | 0.102 | |
| 2216412 | AI654749 | 2.19 | 0.343 | 0.157 | |
| 754929 | AA422036 | 2.18 | 0.38 | 0.174 | |
| 981382 | AA525940 | 2.16 | 0.225 | 0.104 | |
| 1965573 | AI364874 | 2.1 | 0.305 | 0.146 | |
| 981208 | AA526408 | 2.09 | 0.322 | 0.154 | |
| 757030 | AA429021 | 2.08 | 0.344 | 0.165 | |
| 742056 | AA405746 | 2.07 | 0.314 | 0.152 | |
| 1861875 | AI053808 | 2.07 | 0.254 | 0.122 | |
| 1373857 | AA828779 | 2.05 | 0.257 | 0.125 | |
| 1373888 | AA828792 | 2.04 | 0.223 | 0.109 | |
| 810216 | AA463996 | 2.02 | 0.377 | 0.186 | |
| 1374124 | AA828604 | 2.02 | 0.204 | 0.101 | |
| 981025 | AA526011 | 2.01 | 0.349 | 0.173 | |
| 1212476 | AA643476 | 2 | 0.217 | 0.109 | |
| OVM-28 | | 2.63 | 0.313 | 0.119 | OVM-28 |

Threshold >2.0; Mean Signal 2 >0.2; Ranked by ratio.

| | | | | | |
|---|---|---|---|---|---|
| 800175 | AA581095 | 2.93 | 1.213 | 0.414 | |
| 741705 | AA402081 | 2.8 | 3.921 | 1.4 | |
| 740320 | AA477801 | 2.56 | 2.479 | 0.97 | |
| 756537 | AA436442 | 2.42 | 2.437 | 1.007 | |
| 897358 | AA496757 | 2.32 | 1.246 | 0.537 | |
| 1965502 | AI364842 | 2.26 | 0.662 | 0.293 | |
| 755590 | AA419204 | 2.25 | 1.832 | 0.813 | |
| 1964045 | AI280624 | 2.22 | 0.618 | 0.279 | |
| 1965524 | AI364851 | 2.2 | 1.354 | 0.615 | |
| 740416 | AA477828 | 2.11 | 0.477 | 0.226 | |
| 770267 | AA434256 | 2.04 | 0.48 | 0.236 | |
| 811005 | AA485364 | 2.04 | 0.539 | 0.264 | |
| 741183 | AA402548 | 2.03 | 2.903 | 1.431 | |
| 727859 | AA393566 | 2.02 | 0.51 | 0.252 | |
| OVM-61 | | 2.44 | 0.818 | 0.335 | OVM-61 |

Of those sequences presented in Table 7, several that demonstrated good profiles were selected for Real Time PCR analysis and their expression in a selection of normal and tumor tissues. Table 8 describes the expression two of these clones.

TABLE 8
Real Time PCR Expression Profiles
of Sequences Identified on Ovary Chip #4

| | | | Ovary Tumor | |
|---|---|---|---|---|
| | | SEQ ID | Tissue and | |
| Clone ID | Image # | NO | tumor associated tissue | Normal Tissue |
| 1069C | 1374123 | 7388; | OE in 6/13 | OE in skeletal |
| | | 10,894 | | muscle and |
| | | | | brain |
| O1070C | 1985442 | 9581; | OE in 7/13; E in 4/13 | E in colon, |
| | | 10,895 | | brain and |
| | | | | cerebellum |

OE = over expressed; E = expressed

All clones subjected to Real Time PCR, in addition to several other clones identified using microarray, were re-sequenced and the sequences were searched against Genbank to identify extended and/or full length sequences. These sequences are disclosed in SEQ ID NO:10,863-10,912.

### Example 3

### Peptide Priming of T-Helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 4

### Generation of Tumor-Specific CTL Lines Using in Vitro Whole-Gene Priming

Using in vitro whole-gene priming with tumor antigen-vaccinia infected DC (see, for example, Yee et al, The Journal of Immunology, 157(9):4079-86, 1996), human CTL lines are derived that specifically recognize autologous fibroblasts transduced with a specific tumor antigen, as determined by interferon-γ ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8+ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8+ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

### Example 5

### Generation and Characterization of Anti-Tumor Antigen Monoclonal Antibodies

Mouse monoclonal antibodies are raised against E. coli derived tumor antigen proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10 μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the surface of cells stably transfected with the cDNA encoding the tumor protein.

### Example 6

### Synthesis of Polypeptides

Polypeptides are synthesized on a Perkin Elmer/Applied Biosystems Division 430A peptide synthesizer using FMOC chemistry with HPTU (O-Benzotriazole-N,N,N′, N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A method for detecting the presence of ovarian cancer in a patient, comprising the steps of:
(a) obtaining a biological sample from a patient;
(b) contacting the biological sample with an oligonucleotide that hybridizes to a sequence set forth in any one of SEQ ID NO: 1-10,912 under highly stringent conditions;
(c) detecting in the sample an amount of a polynucleotide that hybridizes to the oligonucleotide; and
(d) comparing the amount of polynucleotide that hybridizes to the oligonucleotide to a predetermined cut-off value, and therefrom detecting the presence of ovarian cancer in the patient.

2. The method of claim 1, wherein said sequence is selected from a sequence set forth in any one of SEQ ID NO: 10863-10912.

3. The method of claim 1, wherein said sequence is selected from a sequence set forth in any one of SEQ ID NO: 10864-10869, 10872-10878, 10880-10884 and 10894-10895.

4. The method of claim 1, wherein said detecting in the sample an amount of a polynucleotide that hybridizes to the oligonucleotide is performed by a polymerase chain reaction.

5. The method of claim 1, wherein the biological sample is selected from the group consisting of serum and ovarian tissue.

6. An oligonucleotide useful in the detection of ovarian cancer in a patient, wherein said oligonucleotide hybridizes to a sequence set forth in any one of SEQ ID NO: 1-10,912 under highly stringent conditions.

7. A diagnostic kit comprising at least one oligonucleotide according to claim 6.

8. A method for detecting the presence of a cancer in a patient, comprising the steps of:
(a) obtaining a biological sample from a patient;
(b) contacting the biological sample with a binding agent that binds to a polypeptide selected from the group consisting of:
(i) a polypeptide encoded by a polynucleotide sequence set forth in any one of SEQ ID NO: 1-10,912;
(ii) a sequence having at least 90% identity to said polypeptide;
(iii) a sequence having at least 95% identity to said polypeptide;
(c) detecting in the sample an amount of polypeptide that binds to the binding agent; and
(d) comparing the amount of polypeptide to a predetermined cut-off value and therefrom detecting the presence of a cancer in the patient.

9. A method for stimulating and/or expanding T cells specific for an ovarian tumor protein, comprising contacting T cells with at least one component selected from the group consisting of:
(a) a polypeptide sequence selected from the group consisting of:
(i) a polypeptide encoded by a polynucleotide sequence set forth in any one of SEQ ID NO: 1-10,912;
(ii) a sequence having at least 90% identity to said polypeptide;
(iii) a sequence having at least 95% identity to said polypeptide;
(b) a polynucleotide selected from the group consisting of:
(i) a sequence set forth in any one of SEQ ID NO: 1-10,912;
(ii) a complement of a sequence set forth in any one of SEQ ID NO: 1-10,912;
(iii) a sequence consisting of at least 20 contiguous residues of a sequence set forth in any one of SEQ ID NO: 1-10,912;
(iv) a sequence that hybridizes to a sequence set forth in any one of SEQ ID NO: 1-10,912, under highly stringent conditions;
(v) a sequence having at least 90% identity to a sequence set forth in any one of SEQ ID NO: 1-10,912; and
(vi) a sequence having at least 95% identity to a sequence set forth in any one of SEQ ID NO: 1-10,912.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) a polypeptide sequence selected from the group consisting of:
(i) a polypeptide encoded by a polynucleotide sequence set forth in any one of SEQ ID NO: 1-10,912;
(ii) a sequence having at least 90% identity to said polypeptide;
(iii) a sequence having at least 95% identity to said polypeptide;
(b) a polynucleotide sequence selected from the group consisting of:
(i) a sequence set forth in any one of SEQ ID NO: 1-10,912;
(ii) a complement of a sequence set forth in any one of SEQ ID NO: 1-10,912;
(iii) a sequence consisting of at least 20 contiguous residues of a sequence set forth in any one of SEQ ID NO: 1-10,912;
(iv) a sequence that hybridizes to a sequence set forth in any one of SEQ ID NO: 1-10,912 under highly stringent conditions;
(v) a sequence having at least 95% identity to a sequence set forth in any one of SEQ ID NO: 1-10,912;
(vi) a degenerate variant of a sequence set forth in any one of SEQ ID NO: 1-10,912;
(c) a T cell population according to claim 10; and
(d) antigen presenting cells that express a polypeptide selected from the group consisting of:
(i) a polypeptide encoded by a polynucleotide sequence set forth in any one of SEQ ID NO: 1-10,912.
(ii) a sequence having at least 90% identity to said polypeptide; and
(iii) a sequence having at least 95% identity to said polypeptide.
